SEQUENCE LISTING (325 sequences)

SEQ ID NO: 1 - DNA, 5357 bp, synthetic construct
  source 1..5357: mol_type = other DNA
gacatcatcacctagcagttcatgcagctagcaagtggtttgttcttagggtaacagaggaggaaattgttcctcgtctgataagacaacagtggagaaaggacgcatgctgtttcttagggacacggctgacttccagatatgaccatgtatttgtggcttaaactcttggcatttggctttgcctttctggacacagaagtatttgtgacagggcaaagcccaacaccttcccccactggattgactacagcaaagatgcccagtgttccactttcaagtgaccccttacctactcacaccactgcattctcacccgcaagcacctttgaaagagaaaatgacttctcagagaccacaacttctcttagtccagacaatacttccacccaagtatccccggactctttggataatgctagtgcttttaataccacaggtgtttcatcagtacagacgcctcaccttcccacgcacgcagactcgcagacgccctctgctggaactgacacgcagacattcagcggctccgccgccaatgcaaaactcaaccctaccccaggcagcaatgctatctcagatgtcccaggagagaggagtacagccagcacctttcctacagacccagtttccccattgacaaccaccctcagccttgcacaccacagctctgctgccttacctgcacgcacctccaacaccaccatcacagcgaacacctcagatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctacataagaaaagatcctgcaatttagatgaacagcaggagcttgttgaaagggatgatgaaaaacaactgatgaatgtggagccaatccatgcagatattttgttggaaacttataagaggaagattgctgatgaaggaagactttttctggctgaatttcagagcatcccgcgggtgttcagcaagtttcctataaaggaagctcgaaagccctttaaccagaataaaaaccgttatgttgacattcttccttatgattataaccgtgttgaactctctgagataaacggagatgcagggtcaaactacataaatgccagctatattgatggtttcaaagaacccaggaaatacattgctgcacaaggtcccagggatgaaactgttgatgatttctggaggatgatttgggaacagaaagccacagttattgtcatggtcactcgatgtgaagaaggaaacaggaacaagtgtgcagaatactggccgtcaatggaagagggcactcgggcttttggagatgttgttgtaaagatcaaccagcacaaaagatgtccagattacatcattcagaaattgaacattgtaaataaaaaagaaaaagcaactggaagagaggtgactcacattcagttcaccagctggccagaccacggggtgcctgaggatcctcacttgctcctcaaactgagaaggagagtgaatgccttcagcaatttcttcagtggtcccattgtggtgcactgcagtgctggtgttgggcgcacaggaacctatatcggaattgatgccatgctagaaggcctggaagccgagaacaaagtggatgtttatggttatgttgtcaagctaaggcgacagagatgcctgatggttcaagtagaggcccagtacatcttgatccatcaggctttggtggaatacaatcagtttggagaaacagaagtgaatttgtctgaattacatccatatctacataacatgaagaaaagggatccacccagtgagccgtctccactagaggctgaattccagagacttccttcatataggagctggaggacacagcacattggaaatcaagaagaaaataaaagtaaaaacaggaattctaatgtcatcccatatgactataacagagtgccacttaaacatgagctggaaatgagtaaagagagtgagcatgattcagatgaatcctctgatgatgacagtgattcagaggaaccaagcaaatacatcaatgcatcttttataatgagctactggaaacctgaagtgatgattgctgctcagggaccactgaaggagaccattggtgacttttggcagatgatcttccaaagaaaagtcaaagttattgttatgctgacagaactgaaacatggagaccaggaaatctgtgctcagtactggggagaaggaaagcaaacatatggagatattgaagttgacctgaaagacacagacaaatcttcaacttatacccttcgtgtctttgaactgagacattccaagaggaaagactctcgaactgtgtaccagtaccaatatacaaactggagtgtggagcagcttcctgcagaacccaaggaattaatctctatgattcaggtcgtcaaacaaaaacttccccagaagaattcctctgaagggaacaagcatcacaagagtacacctctactcattcactgcagggatggatctcagcaaacgggaatattttgtgctttgttaaatctcttagaaagtgcggaaacagaagaggtagtggatatttttcaagtggtaaaagctctacgcaaagctaggccaggcatggtttccacattcgagcaatatcaattcctatatgacgtcattgccagcacctaccctgctcagaatggacaagtaaagaaaaacaaccatcaagaagataaaattgaatttgataatgaagtggacaaagtaaagcaggatgctaattgtgttaatccacttggtgccccagaaaagctccctgaagcaaaggaacaggctgaaggttctgaacccacgagtggcactgaggggccagaacattctgtcaatggtcctgcaagtccagctttaaatcaaggttcataggaaaagacataaatgaggaaactccaaacctcctgttagctgttatttctatttttgtagaagtaggaagtgaaaataggtatacagtggattaattaaatgcagcgaaccaatatttgtagaagggttatattttactactgtggaaaaatatttaagatagttttgccagaacagtttgtacagacgtatgcttattttaaaattttatctcttattcagtaaaaaacaacttctttgtaatcgttatgtgtgtatatgtatgtgtgtatgggtgtgtgtttgtgtgagagacagagaaagagagagaattctttcaagtgaatctaaaagcttttgcttttcctttgtttttatgaagaaaaaatacattttatattagaagtgttaacttagcttgaaggatctgtttttaaaaatcataaactgtgtgcagactcaataaaatcatgtacatttctgaaatgacctcaagatgtcctccttgttctactcatatatatctatcttatatagtttactattttacttctagagatagtacataaaggtggtatgtgtgtgtatgctactacaaaaaagttgttaactaaattaacattgggaaatcttatattccatatattagcatttagtccaatgtctttttaagcttatttaattaaaaaatttccagtgagcttatcatgctgtctttacatggggttttcaattttgcatgctcgattattccctgtacaatatttaaaatttattgcttgatacttttgacaacaaattaggttttgtacaattgaacttaaataaatgtcattaaaataaataaatgcaatatgtattaatattcattgtataaaaatagaagaatacaaacatatttgttaaatatttacatatgaaatttaatatagctatttttatggaatttttcattgatatgaaaaatatgatattgcatatgcatagttcccatgttaaatcccattcataactttcattaaagcatttactttgaatttctccaatgcttagaatgtttttaccaggaatggatgtcgctaatcataataaaattcaaccattatttttttcttgtttataatacattgtgttatatgttcaaatatgaaatgtgtatgcacctattgaaatatgtttaatgcatttattaacatttgcaggacacttttacaggccccaattatccaatagtctaataattgtttaagatctagaaaaaaaaaatcaagaatagtggtatttttcatgaagtaataaaaactcgttttggtgaa

SEQ ID NO: 2 - protein, 1748 aa, synthetic construct
DIITQFMQLASGLFLGQRRKLFLVDNSGERTHAVSGHGLPDMTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGSEKTMRKLQTSCLLFLFLKEVKIGIQWINMQRTNICRRVIFYYCGKIFKIVLPEQFVQTYAYFKILSLIQKTTSLSLCVYMYVCMGVCLCERQRKRENSFKIKLLLFLCFYEEKIHFILEVLTLEGSVFKNHKLCADSIKSCTFLKPQDVLLVLLIYIYLIFTILLLEIVHKGGMCVYATTKKLLTKLTLGNLIFHILAFSPMSFAYLIKKFPVSLSCCLYMGFSILHARLFPVQYLKFIAYFQQIRFCTIELKMSLKINAICINIHCIKIEEYKHICIFTYEIYSYFYGIFHYEKYDIAYAFPCIPFITFIKAFTLNFSNANVFTRNGCRSNSTIIFFLFIIHCVICSNMKCVCTYNMFNAFINICRTLLQAPIIQSNNCLRSRKKKSRIVVFFMKKLVLV

SEQ ID NO: 3 - DNA, 4874 bp, synthetic construct
  source 1..4874: mol_type = other DNA
gacatcatcacctagcagttcatgcagctagcaagtggtttgttcttagggtaacagaggaggaaattgttcctcgtctgataagacaacagtggagaaaggacgcatgctgtttcttagggacacggctgacttccagatatgaccatgtatttgtggcttaaactcttggcatttggctttgcctttctggacacagaagtatttgtgacagggcaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctacataagaaaagatcctgcaatttagatgaacagcaggagcttgttgaaagggatgatgaaaaacaactgatgaatgtggagccaatccatgcagatattttgttggaaacttataagaggaagattgctgatgaaggaagactttttctggctgaatttcagagcatcccgcgggtgttcagcaagtttcctataaaggaagctcgaaagccctttaaccagaataaaaaccgttatgttgacattcttccttatgattataaccgtgttgaactctctgagataaacggagatgcagggtcaaactacataaatgccagctatattgatggtttcaaagaacccaggaaatacattgctgcacaaggtcccagggatgaaactgttgatgatttctggaggatgatttgggaacagaaagccacagttattgtcatggtcactcgatgtgaagaaggaaacaggaacaagtgtgcagaatactggccgtcaatggaagagggcactcgggcttttggagatgttgttgtaaagatcaaccagcacaaaagatgtccagattacatcattcagaaattgaacattgtaaataaaaaagaaaaagcaactggaagagaggtgactcacattcagttcaccagctggccagaccacggggtgcctgaggatcctcacttgctcctcaaactgagaaggagagtgaatgccttcagcaatttcttcagtggtcccattgtggtgcactgcagtgctggtgttgggcgcacaggaacctatatcggaattgatgccatgctagaaggcctggaagccgagaacaaagtggatgtttatggttatgttgtcaagctaaggcgacagagatgcctgatggttcaagtagaggcccagtacatcttgatccatcaggctttggtggaatacaatcagtttggagaaacagaagtgaatttgtctgaattacatccatatctacataacatgaagaaaagggatccacccagtgagccgtctccactagaggctgaattccagagacttccttcatataggagctggaggacacagcacattggaaatcaagaagaaaataaaagtaaaaacaggaattctaatgtcatcccatatgactataacagagtgccacttaaacatgagctggaaatgagtaaagagagtgagcatgattcagatgaatcctctgatgatgacagtgattcagaggaaccaagcaaatacatcaatgcatcttttataatgagctactggaaacctgaagtgatgattgctgctcagggaccactgaaggagaccattggtgacttttggcagatgatcttccaaagaaaagtcaaagttattgttatgctgacagaactgaaacatggagaccaggaaatctgtgctcagtactggggagaaggaaagcaaacatatggagatattgaagttgacctgaaagacacagacaaatcttcaacttatacccttcgtgtctttgaactgagacattccaagaggaaagactctcgaactgtgtaccagtaccaatatacaaactggagtgtggagcagcttcctgcagaacccaaggaattaatctctatgattcaggtcgtcaaacaaaaacttccccagaagaattcctctgaagggaacaagcatcacaagagtacacctctactcattcactgcagggatggatctcagcaaacgggaatattttgtgctttgttaaatctcttagaaagtgcggaaacagaagaggtagtggatatttttcaagtggtaaaagctctacgcaaagctaggccaggcatggtttccacattcgagcaatatcaattcctatatgacgtcattgccagcacctaccctgctcagaatggacaagtaaagaaaaacaaccatcaagaagataaaattgaatttgataatgaagtggacaaagtaaagcaggatgctaattgtgttaatccacttggtgccccagaaaagctccctgaagcaaaggaacaggctgaaggttctgaacccacgagtggcactgaggggccagaacattctgtcaatggtcctgcaagtccagctttaaatcaaggttcataggaaaagacataaatgaggaaactccaaacctcctgttagctgttatttctatttttgtagaagtaggaagtgaaaataggtatacagtggattaattaaatgcagcgaaccaatatttgtagaagggttatattttactactgtggaaaaatatttaagatagttttgccagaacagtttgtacagacgtatgcttattttaaaattttatctcttattcagtaaaaaacaacttctttgtaatcgttatgtgtgtatatgtatgtgtgtatgggtgtgtgtttgtgtgagagacagagaaagagagagaattctttcaagtgaatctaaaagcttttgcttttcctttgtttttatgaagaaaaaatacattttatattagaagtgttaacttagcttgaaggatctgtttttaaaaatcataaactgtgtgcagactcaataaaatcatgtacatttctgaaatgacctcaagatgtcctccttgttctactcatatatatctatcttatatagtttactattttacttctagagatagtacataaaggtggtatgtgtgtgtatgctactacaaaaaagttgttaactaaattaacattgggaaatcttatattccatatattagcatttagtccaatgtctttttaagcttatttaattaaaaaatttccagtgagcttatcatgctgtctttacatggggttttcaattttgcatgctcgattattccctgtacaatatttaaaatttattgcttgatacttttgacaacaaattaggttttgtacaattgaacttaaataaatgtcattaaaataaataaatgcaatatgtattaatattcattgtataaaaatagaagaatacaaacatatttgttaaatatttacatatgaaatttaatatagctatttttatggaatttttcattgatatgaaaaatatgatattgcatatgcatagttcccatgttaaatcccattcataactttcattaaagcatttactttgaatttctccaatgcttagaatgtttttaccaggaatggatgtcgctaatcataataaaattcaaccattatttttttcttgtttataatacattgtgttatatgttcaaatatgaaatgtgtatgcacctattgaaatatgtttaatgcatttattaacatttgcaggacacttttacaggccccaattatccaatagtctaataattgtttaagatctagaaaaaaaaaatcaagaatagtggtatttttcatgaagtaataaaaactcgttttggtgaa

SEQ ID NO: 4 - protein, 1587 aa, synthetic construct
DIITQFMQLASGLFLGQRRKLFLVDNSGERTHAVSGHGLPDMTMYLWLKLLAFGFAFLDTEVFVTGQSPTPSPTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGSEKTMRKLQTSCLLFLFLKEVKIGIQWINMQRTNICRRVIFYYCGKIFKIVLPEQFVQTYAYFKILSLIQKTTSLSLCVYMYVCMGVCLCERQRKRENSFKIKLLLFLCFYEEKIHFILEVLTLEGSVFKNHKLCADSIKSCTFLKPQDVLLVLLIYIYLIFTILLLEIVHKGGMCVYATTKKLLTKLTLGNLIFHILAFSPMSFAYLIKKFPVSLSCCLYMGFSILHARLFPVQYLKFIAYFQQIRFCTIELKMSLKINAICINIHCIKIEEYKHICIFTYEIYSYFYGIFHYEKYDIAYAFPCIPFITFIKAFTLNFSNANVFTRNGCRSNSTIIFFLFIIHCVICSNMKCVCTYNMFNAFINICRTLLQAPIIQSNNCLRSRKKKSRIVVFFMKKLVLV

SEQ ID NO: 5 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 6 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 7 - DNA, 1239 bp, synthetic construct
  source 1..1239: mol_type = other DNA
atgaccatgtatttgtggcttaaactcttggcatttggctttgcctttctggacacagaagtatttgtgacaggggaacaaaaactcatctcagaagaggatctgtgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 8 - protein, 412 aa, synthetic construct
MTMYLWLKLLAFGFAFLDTEVFVTGEQKLISEEDLCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 9 - DNA, 1023 bp, synthetic construct
  source 1..1023: mol_type = other DNA
atgaccatgtatttgtggcttaaactcttggcatttggctttgcctttctggacacagaagtatttgtgacaggggaacaaaaactcatctcagaagaggatctgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 10 - protein, 340 aa, synthetic construct
MTMYLWLKLLAFGFAFLDTEVFVTGEQKLISEEDLPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 11 - DNA, 750 bp, synthetic construct
  source 1..750: mol_type = other DNA
atgaccatgtatttgtggcttaaactcttggcatttggctttgcctttctggacacagaagtatttgtgacaggggaacaaaaactcatctcagaagaggatctgagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 12 - protein, 249 aa, synthetic construct
MTMYLWLKLLAFGFAFLDTEVFVTGEQKLISEEDLSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 13 - DNA, 471 bp, synthetic construct
  source 1..471: mol_type = other DNA
atgaccatgtatttgtggcttaaactcttggcatttggctttgcctttctggacacagaagtatttgtgacaggggaacaaaaactcatctcagaagaggatctgcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 14 - protein, 156 aa, synthetic construct
MTMYLWLKLLAFGFAFLDTEVFVTGEQKLISEEDLPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 15 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgcagccgcggcagcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 16 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCAAAAANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 17 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgcggcctgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 18 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDEKYANITVDYLYNKETKLFTAKLNVNENAACGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 19 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtgccgctcctgctgcgacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 20 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDEKYANITVDYLYNKETKLFTAKLNVNENVECGNNTCTNNEVHNLTECKNASVSISHNSCAAPAATLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 21 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttgccgaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 22 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDEKYANITVDYLYNKETKLFTAKLNVNENVECGNNTCTNNEVHNLAECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 23 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcgccgctaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 24 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDEKYANITVDYLYNKETKLFTAKLNVNENVECGNNTCAANEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 25 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttgccgcagctctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 26 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDEKYANITVDYLYNKETKLFAAALNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 27 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaagccgcggcttttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 28 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDEKYANITVDYLYNKEAAAFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 29 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccgcggatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 30 - protein, 453 aa, synthetic construct
MTMYLWLKLLAFGFAFLDTEVFVTGEQKLISEEDLQSPTPSPADAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 31 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacgctgcgtgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 32 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDEKYANITVDYLYNKETKLFTAKLNVNENVECGNAACTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 33 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctagcggttgccgctaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 34 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDEKYANITVDYLYNKETKLFTAKLAVAANVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 35 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatatgccgcggctactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 36 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDEKYANITVDYLYAAATKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 37 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacagcggctttagccgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 38 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDEKYANITVDYLYNKETKLFTAKLNVNENVECGNNTCTNNEVHNLTECKNASVSISHNSCTAPDKTAALAVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 39 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtgccgctgcggccgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 40 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDEKYANITVDYLYNKETKLFTAKLNVNENVECGNNTCTNNEVHNLTECAAAAVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 41 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccgcgtctgccgcttcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 42 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDEKYANITVDYLYNKETKLFTAKLNVNENVECGNNTCTNNEVHNLTECKNASVSASAASCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 43 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgcggccgctaaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 44 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDEKYANITVDYLYNKETKLFTAKLNVNENVECGNNTCTNNAAANLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 45 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggcctacgcgtataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 46 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDEKYANITVAYAYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 47 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgaaaaagccgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 48 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDEKAANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 49 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggccgcagctgctgcgttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 50 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGAAAAALHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 51 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctatgccgctgcagcctttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 52 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYAAAAFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 53 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcatgcttcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 54 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDEKYANITVDYLYNKETKLFTAKLNVNENVECGNNTCTNNEVHNLTECKNASVSISHASCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 55 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgaagccgctgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 56 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDEAAANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 57 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgaagcatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 58 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDEAYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 59 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatgcagccgccgctgcctgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 60 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNAAAAACDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 61 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccgcctctcatgcttcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 62 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDEKYANITVDYLYNKETKLFTAKLNVNENVECGNNTCTNNEVHNLTECKNASVSASHASCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 63 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccgcctctgctaattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 64 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDEKYANITVDYLYNKETKLFTAKLNVNENVECGNNTCTNNEVHNLTECKNASVSASANSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 65 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccgcctctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 66 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDEKYANITVDYLYNKETKLFTAKLNVNENVECGNNTCTNNEVHNLTECKNASVSASHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 67 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctgctgcttcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 68 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDEKYANITVDYLYNKETKLFTAKLNVNENVECGNNTCTNNEVHNLTECKNASVSISAASCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 69 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctgctaattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 70 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDEKYANITVDYLYNKETKLFTAKLNVNENVECGNNTCTNNEVHNLTECKNASVSISANSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 71 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaaggccgctgccgcagccaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 72 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKAAAAAKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 73 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgccaaagctgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 74 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDAKAANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 75 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgccgcttatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 76 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDAAYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 77 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgccaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 78 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDAKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 79 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgccgcagcggctattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 80 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCAAAAITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 81 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgccgaaaaagccgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 82 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCAEKAANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 83 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgccgcaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 84 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCAAKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 85 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgccgaagcctatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 86 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCAEAYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 87 - DNA, 1362 bp, synthetic construct
  source 1..1362: mol_type = other DNA
atgaccatgtatttgtggcttaaactcttggcatttggctttgcctttctggacacagaagtatttgtgacaggggaacaaaaactcatctcagaagaggatctgcaaagcccaacaccttcccccactgatgcctaccttaatgcctctgaaacaaccactctgagcccttctggaagcgctgtcatttcaaccacaacaatagctactactccatctaagccaacatgtgccgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctataa

SEQ ID NO: 88 - protein, 453 aa, synthetic construct
MTMYLWLKLLAFGFAFLDTEVFVTGEQKLISEEDLQSPTPSPTDAYLNASETTTLSPSGSAVISTTTIATTPSKPTCAEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDL

SEQ ID NO: 89 - protein, 5 aa, synthetic construct
SYTMQ

SEQ ID NO: 90 - protein, 17 aa, synthetic construct
YINPSSGYIKYNQKFKD

SEQ ID NO: 91 - protein, 8 aa, synthetic construct
RGSYFFDF

SEQ ID NO: 92 - protein, 5 aa, synthetic construct
SYSIQ

SEQ ID NO: 93 - protein, 17 aa, synthetic construct
YINPSSGYIKYNQHFRD

SEQ ID NO: 94 - protein, 8 aa, synthetic construct
GNSGSFDY

SEQ ID NO: 95 - protein, 11 aa, synthetic construct
GFSFSAGYWIC

SEQ ID NO: 96 - protein, 18 aa, synthetic construct
CTYAGRSGSTYYANWVNG

SEQ ID NO: 97 - protein, 10 aa, synthetic construct
GNAGVAVGAL

SEQ ID NO: 98 - protein, 10 aa, synthetic construct
GFDFSRYWMS

SEQ ID NO: 99 - protein, 17 aa, synthetic construct
EINPTSSTINFTPSLKD

SEQ ID NO: 100 - protein, 10 aa, synthetic construct
GNYYRYGDAM

SEQ ID NO: 101 - protein, 8 aa, synthetic construct
GYTFSGYA

SEQ ID NO: 102 - protein, 8 aa, synthetic construct
INPKSPGT

SEQ ID NO: 103 - protein, 11 aa, synthetic construct
ARYSSSSWGDP

SEQ ID NO: 104 - protein, 5 aa, synthetic construct
GYFIH

SEQ ID NO: 105 - protein, 17 aa, synthetic construct
YISCYNGATSYNQKFKG

SEQ ID NO: 106 - protein, 11 aa, synthetic construct
NYYGNLDAMDY

SEQ ID NO: 107 - protein, 7 aa, synthetic construct
FSNYGMS

SEQ ID NO: 108 - protein, 14 aa, synthetic construct
TIVGNDYTYFPDSM

SEQ ID NO: 109 - protein, 7 aa, synthetic construct
HDWVFDY

SEQ ID NO: 110 - protein, 5 aa, synthetic construct
NYIIH

SEQ ID NO: 111 - protein, 16 aa, synthetic construct
FNPYNHGTKYNEKFKG

SEQ ID NO: 112 - protein, 9 aa, synthetic construct
ARSGPYAWF

SEQ ID NO: 113 - protein, 7 aa, synthetic construct
GFSLTTY

SEQ ID NO: 114 - protein, 7 aa, synthetic construct
IWWNDNK

SEQ ID NO: 115 - protein, 6 aa, synthetic construct
LYGYTY

SEQ ID NO: 116 - protein, 7 aa, synthetic construct
GFSLTTY

SEQ ID NO: 117 - protein, 7 aa, synthetic construct
IWWNDNK

SEQ ID NO: 118 - protein, 6 aa, synthetic construct
LYGYTY

SEQ ID NO: 119 - protein, 16 aa, synthetic construct
RSSKSLLYKDGKTYLN

SEQ ID NO: 120 - protein, 7 aa, synthetic construct
LMSTRAS

SEQ ID NO: 121 - protein, 9 aa, synthetic construct
QQLVEYPFT

SEQ ID NO: 122 - protein, 16 aa, synthetic construct
RSSKSLLHSSGITYLY

SEQ ID NO: 123 - protein, 7 aa, synthetic construct
RMSNLAS

SEQ ID NO: 124 - protein, 9 aa, synthetic construct
MQHLEYPFT

SEQ ID NO: 125 - protein, 11 aa, synthetic construct
QASQSISNWLA

SEQ ID NO: 126 - protein, 7 aa, synthetic construct
QASKLAS

SEQ ID NO: 127 - protein, 13 aa, synthetic construct
QSYYDSGSNVFFA

SEQ ID NO: 128 - protein, 15 aa, synthetic construct
RASKSVSTSGYSYLH

SEQ ID NO: 129 - protein, 7 aa, synthetic construct
LASNLES

SEQ ID NO: 130 - protein, 9 aa, synthetic construct
QHSRELPFT

SEQ ID NO: 131 - protein, 6 aa, synthetic construct
AQPKAY

SEQ ID NO: 132 - skipped

SEQ ID NO: 133 - protein, 10 aa, synthetic construct
YSTDRSGNRV

SEQ ID NO: 134 - protein, 11 aa, synthetic construct
KASQDVSTAVA

SEQ ID NO: 135 - protein, 7 aa, synthetic construct
SASYRYT

SEQ ID NO: 136 - protein, 9 aa, synthetic construct
QQHYSTPRT

SEQ ID NO: 137 - protein, 15 aa, synthetic construct
KASQSVSFAGSSLMH

SEQ ID NO: 138 - protein, 7 aa, synthetic construct
RASDLET

SEQ ID NO: 139 - protein, 9 aa, synthetic construct
QQSREYPYT

SEQ ID NO: 140 - protein, 11 aa, synthetic construct
RASQNIGTSIQ

SEQ ID NO: 141 - protein, 7 aa, synthetic construct
SSSESIS

SEQ ID NO: 142 - protein, 9 aa, synthetic construct
QQSNTWPFT

SEQ ID NO: 143 - protein, 11 aa, synthetic construct
QSLLYSNGNTY

SEQ ID NO: 144 - skipped

SEQ ID NO: 145 - protein, 9 aa, synthetic construct
SQSTHVPWT

SEQ ID NO: 146 - protein, 117 aa, synthetic construct
GVQLQESGAELARPGASVKMSCKASGYTFTSYTMQWVKQRPGQGLEWIGYINPSSGYIKYNQKFKDKVTLTADKSSTTAYMQLSRLTSEDSAVYYCARRGSYFFDFWGQGTSVTVSS

SEQ ID NO: 147 - protein, 117 aa, synthetic construct
QVQLQQLGAELARPGASVKMSCKASGYTFTSYSIQWVKQRPGQGLEWIGYINPSSGYIKYNQHFRDRATLTADRSSSTAYMQLSSLTSEDSAVYYCARGNSGSFDYWGQGTTLTVSS

SEQ ID NO: 148 - protein, 119 aa, synthetic construct
LSLEESGGDLVKPGASLTLTCTASGFSFSAGYWICWVRQAPGKGLEWIACTYAGRSGSTYYANWVNGRFTIPKTSSTTVTLQMTSLSGADTASYFCARGNAGVAVGALWGPGTLVTVSS

SEQ ID NO: 149 - protein, 120 aa, synthetic construct
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVS

SEQ ID NO: 150 - protein, 118 aa, synthetic construct
QVQLVQSGAEVKKPGASVKVSCKASGYTFSGYAMHWVRQAPGQGLEWMGWINPKSPGTNYAQKFQGRVTMTRDTSISTAYMELSRLRSDDTAVYYCARYSSSSWGDPWGQGTLVTVSS

SEQ ID NO: 151 - protein, 120 aa, synthetic construct
EVQLQQSGPELVKTGASVKISCKASGYSFTGYFIHWVKQSHGKSLEWIGYISCYNGATSYNQKFKGKATFTVDTSSSTAYMQFNSVTSEDSAVYYCVRNYYGNLDAMDYWGQGTSVTVSS

SEQ ID NO: 152 - protein, 115 aa, synthetic construct
EVQLVESGGDLVQPGGSLKLSCTASGFTFSNYGMSWIRQTPDKRLEWVATIVGNDYTYFPDSMKGRFTVSRDNAKSILYLQMNSLASADTAMYYCTRHDWVFDYWGQGTPLTVSS

SEQ ID NO: 153 - protein, 118 aa, synthetic construct
QVQLVESGAEVKKPGASVKVSCKASGYTFTNYIIHWVKQEPGQGLEWIGYFNPYNHGTKYNEKFKGRATLTADKSISTAYMELSSLRSEDTAVYYCARSGPYAWFDTWGQGTTVTVSS

SEQ ID NO: 154 - protein, 114 aa, synthetic construct
QITLKESGPGILQPSQTLSLTCSFSGFSLTTYGIGVGWIRQPPGKGLEWLTHIWWNDNKYYNTALRSRLTISKDSSNNQVLLKIANVDTADTATYYCLYGYTYWGQGTLVTVSA

SEQ ID NO: 155 - protein, 114 aa, synthetic construct
QITLKESGPTLVKPTQTLTLTCTFSGFSLTTYGIGVGWIRQPPGKALEWLTHIWWNDNKYYSPSLKSRLTITKDSSKNQVVLTMTNMDPVDTATYYCLYGYTYWGQGTLVTVSA

SEQ ID NO: 156 - protein, 112 aa, synthetic construct
DIVITQDELSNPVTSGESVSISCRSSKSLLYKDGKTYLNWFLQRPGQSPQLLIYLMSTRASGVSDRFSGSGSGTDFTLEISRVKAEDVGVYYCQQLVEYPFTFGGGTKLEVK

SEQ ID NO: 157 - protein, 112 aa, synthetic construct
DIVMTQAAPSVPVTPGESLSISCRSSKSLLHSSGITYLYWFLQRPGQSPQLLIYRMSNLASGVPDRFSGSGSGTAFTLRISRVEAEDVGVYYCMQHLEYPFTFGGGTKLEIK

SEQ ID NO: 158 - protein, 111 aa, synthetic construct
DIVMTQTPASVSEPVGGTVTIMCQASQSISNWLAWYQQKPGQPPKLLIYQASKLASGVPSRFKGSGSGTEYTLTISDLECADAATYYCQSYYDSGSNVFFAFGGGTKVVVE

SEQ ID NO: 159 - protein, 111 aa, synthetic construct
DIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIK

SEQ ID NO: 160 - protein, 106 aa, synthetic construct
SYELTQPPSVSVSPGQTARVTCSGDAQPKAYAYWYQQKSGQAPILVITGDSKRPSGIPERFSGSSSGTMATLTISGAQVEDEADYYCYSTDRSGNRVFGGGTRLTV

SEQ ID NO: 161 - protein, 107 aa, synthetic construct
DIVMTQSHKFMSTSVGDRVSITCKASQDVSTAVAWYQQKPGQSPKILIYSASYRYTGVPDRFTGSGSGTDFTFTISSVQAEDLAVYYCQQHYSTPRTFGGGTKLEIK

SEQ ID NO: 162 - protein, 111 aa, synthetic construct
DIVLTQSPASLAVSLGQRAILSCKASQSVSFAGSSLMHWYQQKPGQQPKLLIYRASDLETGIPTRFSGGGSGTDFTLNIHPVEEDDAATYYCQQSREYPYTFGGGTRLEIK

SEQ ID NO: 163 - protein, 107 aa, synthetic construct
DILLTQSPATLSLSPGERATLSCRASQNIGTSIQWYQQKPGQAPRLLIRSSSESISGISSRFSGSGSGTDFTLTISSLEPEDFAVYYCQQSNTWPFTFGQGTKLEIK

SEQ ID NO: 164 - protein, 112 aa, synthetic construct
DVVMTQTPLSLPVSLGDQASISCRSSQSLLYSNGNTYLHWYLQKPGQSPKLLIYKLSNRFSGVPDRFSGSGSGTDFTLKISRVEAEDLGVYFCSQSTHVPWTFGGGTKLEIK

SEQ ID NO: 165 - protein, 112 aa, synthetic construct
DVVMTQTPLSLPVTLGQPASISCRSSQSLLYSNGNTYLHWYQQRPGQSPRLLIYKLSNRFSGVPDRFSGSGSGTDFTLKISRVEAEDVGVYYCSQSTHVPWTFGGGTKLEIK

SEQ ID NO: 166 - protein, 21 aa, synthetic construct
MALPVTALLLPLALLLHAARP

SEQ ID NO: 167 - protein, 20 aa, synthetic construct
GGGGSGGGGSGGGGSGGGGS

SEQ ID NO: 168 - protein, 45 aa, synthetic construct
TTTPAPRPPTPAPTIASQPLSLRPEACRPAAGGAVHTRGLDFACD

SEQ ID NO: 169 - protein, 24 aa, synthetic construct
IYIWAPLAGTCGVLLLSLVITLYC

SEQ ID NO: 170 - protein, 42 aa, synthetic construct
KRGRKKLLYIFKQPFMRPVQTTQEEDGCSCRFPEEEEGGCEL

SEQ ID NO: 171 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 172 - protein, 22 aa, synthetic construct
GSGATNFSLLKQAGDVEENPGP

SEQ ID NO: 173 - protein, 277 aa, synthetic construct [sequence omitted]

SEQ ID NO: 174 - DNA, 2379 bp, synthetic construct
  source 1..2379: mol_type = other DNA
atggcgctccctgtgacagccctgctgctgcctttggcgctgctgcttcacgccgctcgtccgggggttcagttacaggagtcgggagcggagctggcccgccctggcgcctccgtgaagatgtcttgcaaggccagcggctacaccttcacatcctacactatgcagtgggtcaaacagcggcccggccagggactggagtggatcggttatattaatccctcttctggctacatcaagtacaaccagaaatttaaggacaaggtcaccctgacagcggataaaagctccaccaccgcttacatgcagctgtcccgtctgacctccgaggattccgccgtgtactattgcgcccgtcgcgggagctacttcttcgatttttggggccagggtacaagcgtcacagtgtcttccggcggcggtggctcgggtggaggcggctccggcggtggtggtagcggaggcggcggttcagacatcgtgatcacccaggacgagttgtcaaatccagtgactagtggagagtccgtgtccatctcctgtcgctcctccaagtcgctgttatataaggatggcaagacttacctgaactggttcctgcagcgtcctgggcagagcccccagctccttatctacctgatgtctacgcgagccagtggcgtgtcagaccgcttttccggttccggcagcgggaccgacttcactctcgagatctccagggtgaaggctgaggacgtaggcgtgtactattgtcagcagctcgtggagtaccctttcaccttcggcggcggcaccaaactggaggtgaagaccactacccccgctccccggccgccgacccccgcccctaccatcgcctctcagccactttcgctacgccctgaggcttgtcggcccgctgccgggggcgcggtgcacacccgcggcctggacttcgcctgcgacatctacatctgggcgccgctggccggcacctgcggggtcctgttgctttctctggtgatcacgctgtattgtaagagaggccgcaagaagctgctgtatatcttcaaacagcccttcatgcgcccagtgcagacgacccaggaggaagatggatgctcttgccgcttccccgaagaggaggagggcgggtgcgagctcagagtgaaattttcccgaagcgcggacgctccggcatacaaacaaggacagaatcagctgtacaacgagttgaacttgggtcgccgggaggaatacgacgtgctggacaagcgccgcggcagggaccctgagatgggagggaagccccgccgcaagaacccccaggagggcctgtacaacgagctgcagaaggacaaaatggccgaagcctactccgagattggtatgaagggcgagcggcgcagaggcaagggtcacgacggcctgtaccagggcttgtctaccgccactaaggacacctacgatgcgctgcacatgcaggcactgcctccccgcggatcgggagctaccaacttttcactgctcaagcaggcgggcgacgtggaggagaacccgggaccgatgggcgcgggcgccacgggacgtgccatggatggtccccgtctacttcttctgttgctactcggcgtgtctcttgggggcgccaaggaggcctgcccgaccgggttgtacacccatagcggcgagtgttgcaaggcctgtaacctgggcgagggcgtcgctcagccctgtggagctaaccagaccgtctgtgagccttgcctggatagcgtgaccttcagtgatgtggtttccgccaccgagccatgcaagccctgcactgagtgcgtgggactccagtcgatgtctgcaccttgcgtggaggccgatgacgccgtgtgccgctgtgcttacggctactaccaagacgagaccaccggtaggtgcgaggcatgccgcgtctgtgaagcgggaagcggactggtgttcagctgtcaggacaagcagaacaccgtttgtgaggagtgcccagacggcacctatagtgacgaggccaatcatgtggacccctgcctgccctgtaccgtgtgcgaggacaccgagcgccagctccgcgagtgtactcgctgggcggacgcagaatgcgaggagattccgggtcgctggataacgaggtccactccgcctgaaggctccgacagtacggctccgagtacccaggagcccgaggctccacccgaacaggacctgatcgcctctaccgtggctggcgtcgttactaccgtgatgggctcctcccaaccagtagtcacccgcggcactactgacaaccttattccggtgtactgttccatcctggcagcagtcgttgtggggctggtggcgtacattgcgttcaagcgatggaactcctga

SEQ ID NO: 175 - protein, 792 aa, synthetic construct
MALPVTALLLPLALLLHAARPGVQLQESGAELARPGASVKMSCKASGYTFTSYTMQWVKQRPGQGLEWIGYINPSSGYIKYNQKFKDKVTLTADKSSTTAYMQLSRLTSEDSAVYYCARRGSYFFDFWGQGTSVTVSSGGGGSGGGGSGGGGSGGGGSDIVITQDELSNPVTSGESVSISCRSSKSLLYKDGKTYLNWFLQRPGQSPQLLIYLMSTRASGVSDRFSGSGSGTDFTLEISRVKAEDVGVYYCQQLVEYPFTFGGGTKLEVKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 176 - DNA, 2379 bp, synthetic construct
  source 1..2379: mol_type = other DNA
atggcgctgcccgtgactgctctgctgttaccgctggcgttgttgctacacgccgccaggcccgacatcgtgatcacccaggatgagttatcaaatcctgttacctccggggagagcgtgtccatctcttgtaggtcctccaagagcttgctttacaaggacggcaagacctacctgaattggttcctgcaacggccaggtcagagcccgcagcttctgatctacctgatgagtactagagcctctggcgtttcggaccgcttctcgggctcggggtccggcaccgacttcaccctggagatctcgcgcgttaaggcggaagacgtgggggtgtactactgtcaacagttagtggaataccccttcaccttcggcggaggaaccaagctggaggtgaaaggcggcggtggctcggggggcggtggctccgggggaggaggttccggcggagggggttccggcgtacagcttcaggagagcggtgctgaactggctaggcccggggcttctgtgaagatgagctgcaaggctagcggctacaccttcaccagttacaccatgcagtgggtcaagcagcgcccgggccagggcctggagtggatcggctacattaatccctcttctgggtacatcaagtacaaccagaaattcaaagataaagtgactctcactgcggacaagtcctcgacgacagcgtatatgcagctgagccgcctgaccagcgaggacagcgcggtgtactactgcgcgcgccgcgggagctacttcttcgacttttggggccagggcacatccgtcaccgtgtcctctaccaccacacccgcgccgcgccctcctactccagctcccaccattgcctcccagcctctttcgctgcgcccagaggcttgtcggcccgcggccgggggagcagtgcacacccgcggcttggatttcgcttgcgatatatatatttgggcgcctctggccggcacctgcggtgtactactgctgtcgttggtgatcaccctctactgcaagcgtggccgcaagaagctcctctacatcttcaaacagcccttcatgcgtcccgtccagactacccaggaggaggatggttgttcttgtcggtttcccgaggaagaggagggcggctgtgagctcagagtcaaattttcccgctcggccgacgctccggcctacaagcagggccagaaccagctgtataacgagctcaacctgggacgccgcgaagagtacgatgtcctggacaagcgacgaggccgtgacccggaaatgggcggcaagccccgccgcaaaaaccctcaggagggcctgtataacgagctacagaaggacaaaatggccgaggcctacagtgagatcggtatgaagggagagcgtcgccgcggtaagggccacgacggattgtatcaaggtctgtctactgcgactaaggacacgtacgacgcgctccatatgcaggccctgcccccccgcggtagcggtgccacgaacttttcactgctcaagcaggctggcgacgtggaggagaaccctggacccatgggggccggcgcaaccggccgggccatggatggccctcgcctgctgctgttactgctccttggcgtgagtctaggcggcgccaaggaggcgtgcccgaccggcttatacacccattccggggagtgttgcaaggcctgtaacctgggcgagggagtggcacagccatgcggggccaaccagacagtgtgcgagccgtgcctggactcagtgaccttttccgacgtggtgtccgctacagagccatgcaagccatgcactgagtgcgtggggctgcagtctatgagcgccccttgcgtggaggctgatgacgccgtgtgcaggtgtgcctacggctactaccaggacgagaccacaggccgctgcgaggcatgtcgcgtttgcgaggctggctccggcctggtgttttcctgccaggacaagcagaacacggtatgtgaagagtgcccagatggcacgtacagcgacgaggctaatcacgtggatccgtgcctgccttgcactgtctgtgaggacaccgagcgccagctgcgagagtgtacccgttgggccgatgctgagtgtgaagagattcccggccggtggatcacccgcagcacgcctcctgagggctctgactccaccgccccctccacccaggagcccgaggccccgcccgaacaggacctcatcgcatctacggtggcaggtgtcgtgactaccgtgatgggatcttcccagcccgtggtcaccagaggtaccacggacaacctgatcccggtgtattgttcgatcctggctgccgttgtcgtcggcctggtagcctatattgcgttcaagcgttggaactcctga

SEQ ID NO: 177 - protein, 792 aa, synthetic construct
MALPVTALLLPLALLLHAARPDIVITQDELSNPVTSGESVSISCRSSKSLLYKDGKTYLNWFLQRPGQSPQLLIYLMSTRASGVSDRFSGSGSGTDFTLEISRVKAEDVGVYYCQQLVEYPFTFGGGTKLEVKGGGGSGGGGSGGGGSGGGGSGVQLQESGAELARPGASVKMSCKASGYTFTSYTMQWVKQRPGQGLEWIGYINPSSGYIKYNQKFKDKVTLTADKSSTTAYMQLSRLTSEDSAVYYCARRGSYFFDFWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 178 - DNA, 2379 bp, synthetic construct
  source 1..2379: mol_type = other DNA
atggcgctgcccgtgacagcccttctgctgcccctggcgctgctcctgcacgccgctaggcctcaggtgcagctccagcagctgggagccgagctggcccgccccggcgcttccgtgaagatgtcttgcaaagcatccggttacactttcacctcgtacagcatccagtgggtgaagcagcgccccggacagggcctggagtggatcggctatattaatccatcatcggggtatatcaagtacaaccagcactttcgtgaccgcgccacccttactgcggaccgctcctcttcaaccgcctacatgcaactgagtagcctgacctccgaagacagcgccgtgtactactgcgctcgtgggaatagtgggtccttcgactattggggccagggaaccaccctgactgtttcctctggcggcggcggttctggcggcggcggtagcggcggaggtggctccggtgggggcggctccgacatcgtcatgacccaggcagccccctccgtgcccgtgaccccgggagagagccttagcatcagctgtcgatccagcaagtccttgctccacagctctggcatcacgtacctatattggttcctgcagagacccggacagagcccacagcttctcatttaccgcatgtctaacttggcttctggggttccggacaggttttcgggatccgggagcggcactgcgttcactctgcgcatctcccgcgtggaggccgaagatgtgggagtttactactgcatgcagcacctggagtacccatttaccttcggcggtggtaccaagctcgagattaagacgacaaccccggccccgcgtcctcctacccccgcccccaccattgcaagccagccactgtctctgcgcccggaggcgtgccgacccgctgcaggtggtgccgtgcacactcgcggcctcgatttcgcttgcgacatctacatctgggctccattggccgggacctgcggggtattgctgctgtccctggtgatcactctttactgcaagcgcggccggaaaaagctgctctacatcttcaagcaacccttcatgaggcctgtccagactacccaggaggaagacggctgttcttgccgcttccctgaagaggaggaaggtggctgcgagctgcgggtgaaattttcccgcagtgcggacgcccccgcctacaagcagggccagaaccagctgtacaacgagctcaacctgggccgccgcgaggagtacgacgtgctggacaaacggcgcggccgtgatccggagatgggtggcaagcctcgtagaaagaatcctcaggagggcctgtacaacgagctgcagaaggataaaatggccgaggcgtactcggagatcgggatgaaaggcgagcgccgtaggggaaagggccatgacggtctctatcagggattgtctaccgccacgaaggacacctatgacgcactgcatatgcaggccctcccgccccggggttccggcgccaccaacttttcactgctgaagcaggctggtgacgtggaggagaatccaggccccatgggggccggcgccaccgggcgcgccatggatggaccccggctgttattactgctgctgcttggcgtgtccctaggcggggccaaggaggcttgtcctaccgggctctacacccactcgggggaatgttgtaaggcctgtaacctgggcgagggcgtggcgcagccgtgcggggcgaaccagacggtctgtgagccatgcttggacagtgtgaccttctctgatgtggtgtccgccaccgagccttgtaagccgtgcacagagtgcgtgggcctgcaatccatgagcgcgccgtgcgtggaggcagatgacgctgtctgccgctgtgcttacggctattaccaggatgagactaccggcaggtgtgaggcttgccgcgtatgtgaggcgggctcgggccttgtattctcttgccaggacaagcagaacacggtgtgcgaggagtgtcccgacggcacctacagtgacgaggccaaccacgtggacccctgcctgccgtgcaccgtgtgcgaggacaccgagcgacagttgcgcgagtgcactcgttgggctgacgcggaatgtgaggagatccccggtcgctggatcacccgctccacccctcctgaaggatccgattcgactgccccctctacccaggagcccgaggctccccctgagcaggacctgattgcttcgactgtcgctggcgttgtcaccacagtcatgggctcatcgcagccggtcgtcaccaggggcacgaccgacaacctgatacccgtgtactgctctatcctggcagccgtggtggtcggcctggtggcttacatcgcgttcaaacgctggaactcctga

SEQ ID NO: 179 - protein, 792 aa, synthetic construct
MALPVTALLLPLALLLHAARPQVQLQQLGAELARPGASVKMSCKASGYTFTSYSIQWVKQRPGQGLEWIGYINPSSGYIKYNQHFRDRATLTADRSSSTAYMQLSSLTSEDSAVYYCARGNSGSFDYWGQGTTLTVSSGGGGSGGGGSGGGGSGGGGSDIVMTQAAPSVPVTPGESLSISCRSSKSLLHSSGITYLYWFLQRPGQSPQLLIYRMSNLASGVPDRFSGSGSGTAFTLRISRVEAEDVGVYYCMQHLEYPFTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 180 - DNA, 2379 bp, synthetic construct
  source 1..2379: mol_type = other DNA
atggctctgcccgttaccgcgctgctcctccccctggctctgctgcttcacgccgctcgcccggacatcgtgatgacccaggccgcacccagcgtgcccgtgacccccggggagagcctgtccatctcgtgccgatcctccaagtccctgttgcacagttcgggcatcacctacctgtactggttcctgcagaggcccggccagagccctcagctgctcatttacaggatgtctaacctggcttccggtgtgcccgaccgcttcagcggctcgggcagtggaaccgcgttcacgctgcgcatctctcgcgtggaggccgaggacgtgggcgtgtactactgtatgcagcacctggagtatcccttcaccttcggcggcggcaccaagctggagatcaaaggcggtggcggctcgggcggaggcggaagtggaggcggcggttcgggagggggtggttcccaggtgcagctccagcagttaggtgccgagctggcgcggcctggtgcttccgtaaaaatgagctgtaaggcgtctgggtacacgttcaccagctacagcatccagtgggtcaagcaacgccccggacagggccttgaatggattggctacattaatccctcttctggctatattaagtacaaccagcactttcgtgaccgcgctaccctcactgctgatcgctcttccagcaccgcctacatgcagttgtcttcactaacctccgaggactccgccgtatactactgcgcccgcggcaactccgggtccttcgactactggggccagggcacaaccctgaccgtcagctcgacgacgacgcctgccccgcgcccccccactccggctcctaccatcgccagccagccactttctctgcgacccgaggcctgccgccccgctgctggtggcgctgtccacacccgcggcctcgattttgcatgtgacatctacatctgggctcccctggccggcacctgcggggttctgttactgtccctggtgatcactctttattgcaaacgtggcagaaagaagctgctgtacatcttcaaacagccgtttatgcgtcctgtgcagacgacccaggaggaagatgggtgctcttgccgtttccccgaggaggaggagggcgggtgcgagctgagagtgaaattttctcgcagcgcagacgctccggcctacaagcagggtcagaaccagctgtacaacgagctcaacctcggacgccgggaggagtacgatgtgctggacaaaagacgtgggcgggatccagagatggggggaaagccccgcaggaagaaccctcaggaaggactatacaacgagctgcagaaggataagatggcggaggcctacagcgaaatcggtatgaaaggcgagcggcgccgcgggaagggtcatgacggactgtatcaaggactgtccactgctactaaggatacctacgacgcgctccacatgcaggccctgcccccgcggggctctggcgcgaccaactttagcctactcaagcaggccggggacgtggaggagaacccggggccaatgggggcaggtgccaccggtcgcgctatggatggcccccgcctgttgctgttgctgctgctgggcgtttcgttgggcggcgccaaggaggcttgtcccaccggcctttacacccattccggtgagtgttgcaaggcctgcaacctgggcgagggcgtcgcccagccttgcggggccaaccagaccgtgtgcgaaccttgcctcgacagcgtcaccttctccgacgtggtgtcagcgactgagccatgtaagccatgcacggagtgcgtgggtcttcagtccatgtccgccccttgcgtggaggccgatgacgccgtctgtaggtgtgcttacggctactaccaggacgagacgacaggtcgttgcgaggcttgtcgtgtatgcgaggcaggatccggcctggtgttctcttgtcaggataagcagaacactgtctgtgaggaatgcccagacggcacttacagcgacgaggccaatcacgtggacccgtgcctgccgtgcactgtctgtgaagacaccgagcgccagctgcgcgagtgtacccgttgggcggacgcagagtgcgaggagatccctggccgctggattacccgctcgacacctcctgagggctctgacagcacagccccatccacccaggaaccggaggcgcccccggagcaggacctgatagcctctaccgtggcgggtgtcgtcaccaccgtgatgggttcgtcacagcccgtagtgactcgcggcaccaccgacaacttgatcccggtgtattgctccatcctggcggcggtggtcgtgggccttgtggcctatatcgcgttcaagcgttggaattcctga

SEQ ID NO: 181 - protein, 792 aa, synthetic construct
MALPVTALLLPLALLLHAARPDIVMTQAAPSVPVTPGESLSISCRSSKSLLHSSGITYLYWFLQRPGQSPQLLIYRMSNLASGVPDRFSGSGSGTAFTLRISRVEAEDVGVYYCMQHLEYPFTFGGGTKLEIKGGGGSGGGGSGGGGSGGGGSQVQLQQLGAELARPGASVKMSCKASGYTFTSYSIQWVKQRPGQGLEWIGYINPSSGYIKYNQHFRDRATLTADRSSSTAYMQLSSLTSEDSAVYYCARGNSGSFDYWGQGTTLTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 182 - DNA, 2382 bp, synthetic construct
  source 1..2382: mol_type = other DNA
atggcccttccggtgactgcgctgctgttaccactggcgctgcttctacacgccgccaggcccctgtccctggaggaatccggaggggacctagtgaagcctggcgcttccctcaccctgacctgcactgcctcgggcttctccttcagtgcaggctattggatttgctgggtgcgccaggctcccggcaaaggcctggagtggattgcgtgcacttacgcgggccgcagtggatcgacctactatgccaactgggtcaacgggcgtttcaccatcccgaagacctccagcacgaccgtcacccttcagatgaccagcttatcaggcgctgacaccgctagctatttctgcgcccggggcaacgcgggtgtagcggtgggtgctctttggggccctggtacattggttactgtcagctctggcgggggaggctctggcggcggtggtagcgggggcggaggctctggcggtgggggttccgacatcgtgatgacccagacccctgcctccgtttcggaaccagtcggcggaaccgtcactatcatgtgccaggcgtcccagagcatctcaaattggctagcctggtaccagcagaagcccggacagccccccaagctgttaatctaccaggcatccaagctggcctccggagtcccgtcgcgctttaagggttctggctctgggactgagtacaccctgaccatttctgacctcgagtgtgccgatgcggctacatattactgtcaaagctactacgatagtggcagcaacgtgttttttgctttcgggggaggtaccaaggtggtcgtggagactaccaccccggcaccccggccgcctaccccagcgcccaccatcgcttctcaacccctgtctctgcgccccgaggcctgccgtccggctgctggtggcgccgtacacacccgcggtttggacttcgcatgcgacatctacatatgggcccccttggccggcacctgcggggtgttactgctcagcctggtgattacactgtattgtaagcgcggccgcaagaagctcttgtacatcttcaaacagccgttcatgcgccctgtgcagacgacccaggaggaagacgggtgttcttgccgcttccccgaggaggaggagggtgggtgcgagctgcgcgtgaaattttccaggagcgcggacgcacccgcgtacaagcagggccagaaccagttgtacaacgagctcaacctgggcagacgggaggagtacgacgtgctggacaagcgccgtggacgcgatccagagatgggcggtaagccgcgacgcaagaaccctcaggagggcctgtacaacgagctgcagaaggacaaaatggcggaggcatactccgagatcggtatgaagggcgagcggcgccgaggcaaggggcatgacggcctctaccagggcctgtcaactgccactaaggacacctacgacgctctgcacatgcaggcccttcccccgcgcggctccggtgccaccaacttttctttgctgaagcaggctggggatgtcgaggaaaaccctggccccatgggcgctggagcaaccggcagagccatggatgggccccgcctgctgctgctgttgctgctgggggtatcgcttggtggcgccaaggaggcctgcccaaccggtctgtacacgcactccggggagtgctgtaaagcgtgtaatctgggcgagggcgtagctcagccgtgcggggctaaccagactgtttgtgaaccctgtctggactcggtgaccttctcggatgtggtttccgccacggagccatgcaaaccttgtacggagtgcgtgggactccagtccatgtccgcaccgtgcgtggaggccgatgacgccgtgtgccgatgcgcgtacggctactaccaagacgagaccactggcaggtgtgaggcctgtcgcgtctgtgaggctggatcgggcctcgtgttctcgtgtcaggataaacagaacacggtgtgcgaggagtgccccgacggcacctattccgacgaagccaatcatgtggacccgtgtctgccctgcactgtctgcgaggacaccgagcgccagctgcgggagtgtacccgctgggcggatgccgagtgcgaggagatccctgggcgttggatcaccaggagcacacctcctgagggctccgacagcacggcgccctctacccaggagcctgaagcgcccccggaacaggacttaatcgccagcaccgtggccggcgtcgtcaccactgtgatgggcagttcccagccggtggttacccgtggcacaaccgacaacctgattccagtgtactgctctatcctggctgccgtagtggtgggcctggtggcttacatcgcgttcaagcgttggaattcctga

SEQ ID NO: 183 - protein, 793 aa, synthetic construct
MALPVTALLLPLALLLHAARPLSLEESGGDLVKPGASLTLTCTASGFSFSAGYWICWVRQAPGKGLEWIACTYAGRSGSTYYANWVNGRFTIPKTSSTTVTLQMTSLSGADTASYFCARGNAGVAVGALWGPGTLVTVSSGGGGSGGGGSGGGGSGGGGSDIVMTQTPASVSEPVGGTVTIMCQASQSISNWLAWYQQKPGQPPKLLIYQASKLASGVPSRFKGSGSGTEYTLTISDLECADAATYYCQSYYDSGSNVFFAFGGGTKVVVETTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 184 - DNA, 2382 bp, synthetic construct
  source 1..2382: mol_type = other DNA
atggctctgcctgtgaccgcactgctgctcccgctggcgctgttgctccacgccgctcggcccgacatcgtgatgacccagacaccagcgagtgtgagcgagccagtgggtggcacagtcactatcatgtgccaggcttcacagtccatctcaaattggctcgcttggtaccagcagaagccgggacagccgcccaagctgctgatttatcaggcttccaagctggcctccggtgtgccaagccgcttcaaaggcagcgggtctggtactgagtacaccctgaccatcagtgacctggagtgcgcggacgccgctacctattactgtcagagctactacgacagcggcagcaacgtgttctttgccttcggcgggggaaccaaggtggtggtggagggcggcgggggatccggtggtggcggctccggcggcggcggctccggtggaggaggaagtttgtctcttgaggagagcgggggggacctggtgaagcccggcgcctccctgaccctgacctgtacggcttcgggcttctccttctcggctgggtattggatttgttgggtgcggcaggccccaggcaaaggcttggagtggatcgcctgcacatatgcgggccgcagcggctcgacttactacgccaactgggtcaacggccgcttcaccatcccgaagaccagctccacgaccgtcactcttcagatgacctctctgtccggtgcggacaccgcttcgtatttctgtgcacgcggtaacgcgggcgtggcggtgggcgcgctttggggccccggcactctggttaccgtgtcctccaccactactcccgccccgcgccctcctaccccggcccccaccattgctagtcagccacttagcctgaggcccgaggcgtgccgacctgccgccggcggcgccgtccacactcgcggtttggactttgcctgtgacatctacatatgggcgcccctggccgggacctgcggggtgctgctgctttccctcgtgattacgctgtactgtaaacgcggtcgcaagaagctgttatatatcttcaagcagcccttcatgcgccccgtgcagactacccaggaggaggacgggtgctcatgccgctttcctgaggaagaggagggcgggtgcgagctgcgggtaaaattttccagaagtgcggacgcacctgcctacaagcaaggccagaaccagctctacaacgagctcaacctgggccgcagagaggagtacgatgtccttgacaagcgtcgtggccgggacccggagatgggcggcaagccgcgcagaaagaaccctcaggaaggcctttacaacgagctgcagaaggacaaaatggccgaggcttactctgagatcggtatgaagggagagcgccgccgcgggaagggtcacgatggactgtaccagggcctgtccaccgctactaaggacacctacgacgccctccatatgcaggccttgcccccgcgtggttctggcgccaccaacttttccctactgaaacaggccggggatgtggaggagaacccaggccctatgggcgctggtgcgaccggccgagccatggatggacccaggctactgctgctattgctgctgggcgtgtccctaggcggagccaaggaggcctgcccaaccggcctctacacgcattctggagagtgttgcaaggcctgcaacctgggcgagggcgtggcacagccttgtggcgccaatcaaactgtatgcgagccatgcctggattccgtgaccttctctgatgttgtcagtgctaccgagccctgtaagccctgcacggagtgcgtggggttgcagtctatgtctgcgccgtgcgtggaggccgatgacgctgtctgtaggtgtgcctacggctactaccaggatgagaccaccggccgatgcgaggcttgcagggtctgcgaggctggttctgggctggtgttctcctgtcaggacaaacagaacacggtctgtgaagaatgcccagacggcacctactccgacgaggccaatcacgtggacccctgcctgccctgcactgtctgtgaagacacggagcgtcagctccgcgagtgcactcgctgggctgacgcagaatgtgaggagatccctgggcgttggatcacgcgctccacaccgcctgagggcagcgattctaccgccccctctacccaggagcccgaagcgcctcctgaacaggacctgatcgcctccaccgtggctggagttgtcacaaccgtgatgggcagctcccaaccggtggtgacccgcggaacaaccgacaacctgattcccgtgtactgctccatcctggcggcagtggttgtaggcttagtcgcatacatcgcgttcaagcgttggaactcctga

SEQ ID NO: 185 - protein, 793 aa, synthetic construct
MALPVTALLLPLALLLHAARPDIVMTQTPASVSEPVGGTVTIMCQASQSISNWLAWYQQKPGQPPKLLIYQASKLASGVPSRFKGSGSGTEYTLTISDLECADAATYYCQSYYDSGSNVFFAFGGGTKVVVEGGGGSGGGGSGGGGSGGGGSLSLEESGGDLVKPGASLTLTCTASGFSFSAGYWICWVRQAPGKGLEWIACTYAGRSGSTYYANWVNGRFTIPKTSSTTVTLQMTSLSGADTASYFCARGNAGVAVGALWGPGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 186 - DNA, 2385 bp, synthetic construct
  source 1..2385: mol_type = other DNA
atggccctgccagtcaccgccctccttctgcccctggcgctccttctgcacgccgctcgccctcaggtccagttggtggagagcggtggcgggctggtgcagcccggggggagcctaaagctcagctgtgcggcttctggtttcgacttttcccgctattggatgtcttgggtccggcaggccccgggcaaaggcctggagtggattggagagattaatccaacctccagcactattaatttcaccccctctcttaaggataaagtgttcatctcacgagataacgcgaagaacacgctgtacctgcagatgtctaaggtgcgctcggaggacaccgccctgtactactgcgcccgcggcaactactaccgctacggcgatgccatggactattggggccaggggaccagcgtgacagtctcgggcggtggcggctccgggggcggtggctccgggggcggaggttcgggcggcggtggctctgacatcgttctgacccagtcaccagcgtccctggccgtatccctggggcagcgcgccaccatctcatgtagggcttccaagtctgtttcgacctccggctatagctacctacattggtaccagcagaagcctggacagccgcccaagctgctgatttacctggccagcaacttagagagtggagtaccagctcgcttcagcggctcgggcagcggtacagacttcaccctcaacatccaccccgtcgaagaggaagacgctgccacttactactgtcaacactcacgagagctccccttcaccttcggctctggtaccaagctggagatcaagaccacaactcctgctcctcgccctccgaccccggctcctaccatcgctagccagccactatcccttcggcccgaagcgtgtaggcccgcagccggcggtgcggtccacacgcggggtttggacttcgcatgtgacatctacatctgggctcctctagcgggtacctgcggagtgctgttgctgtccctcgtgatcaccctttactgcaaacgaggccggaagaagctactgtacatcttcaaacagccttttatgcgcccggtgcagacgacccaggaggaggacggctgttcttgccgctttcccgaagaggaggagggcgggtgcgagctgcgcgtgaaattttcgcgcagtgcggacgccccggcctacaagcagggtcagaaccagctgtacaacgagctgaacctgggacgccgtgaggagtacgacgtgctggacaagcgccgtgggcgggacccggagatgggcggtaagccgagacgcaaaaaccctcaagaggggctctataacgagttgcagaaggacaaaatggctgaggcttacagtgagatcggaatgaagggggagcgccgccgcggcaagggccacgatggcctctatcagggactgtccaccgcgactaaggacacctacgatgctctgcatatgcaggcacttccaccgcgcggctccggagccaccaacttttccctgctgaagcaagcgggcgatgtggaggagaaccccgggcccatgggtgccggcgccacaggccgtgccatggatggaccccgtctgcttctcctgcttttgttgggggtctccttgggaggcgctaaggaggcatgcccaacgggtctttacacccactccggcgaatgttgcaaggcctgcaacctgggcgagggcgtggcgcagccgtgcggggccaaccagaccgtgtgcgagccctgcctggattccgtaactttctctgacgtggtctccgctaccgagccttgtaagccctgcacagagtgcgtgggcctgcagtctatgtccgctccttgcgtggaggcagatgacgcagtgtgccgctgtgcctacggctactatcaagacgagactaccggcagatgtgaggcgtgcagagtttgtgaagcaggcagcggtctcgtcttctcgtgtcaggacaagcagaacactgtatgcgaggagtgtcccgacggcacctactccgacgaggcgaatcacgtggatccatgcctgccctgcactgtctgcgaggacacggagcgccagctgcgcgagtgtacgagatgggcagacgccgagtgcgaggagataccaggtaggtggattactcgctccaccccgcccgaaggcagtgactccaccgctcctagcacccaggagccggaggcgcctccggaacaggacctgatcgccagcacggtggccggcgtggttacaaccgtgatgggatcttcccagcccgtggtgacccgcgggaccactgacaacctgatccccgtgtactgctctatcctggccgctgtggtggtgggcctggtggcctatatcgcgttcaagcgttggaattcctga

SEQ ID NO: 187 - protein, 794 aa, synthetic construct
MALPVTALLLPLALLLHAARPQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSGGGGSGGGGSGGGGSGGGGSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 188 - DNA, 2367 bp, synthetic construct
  source 1..2367: mol_type = other DNA
atggctctccccgtcacagctctgctgctgcccctggccctcctccttcacgctgcccgcccccaggtccagctggtgcagagcggagccgaggtgaagaagcctggggcttctgtgaaggtgtcgtgtaaagcttccggctacacgttctctggttacgcaatgcactgggtgcgccaggctcctggtcagggactggagtggatggggtggataaaccccaagagccccggcaccaactacgcgcagaagttccagggccgagtcactatgacccgcgacacgagcatcagcacggcctacatggaactgagccgcctgcgctccgatgacactgcagtgtattactgcgcccgctactcgagttccagttggggcgacccgtggggacagggtacactggtgaccgtgtcttccggtgggggcggaagcggtggtggtggcagtggaggcggaggctccggaggcggtggctcgagctacgagttgacccagccgccatccgtgagcgtgagccccggccagactgccagagtcacctgttctggggacgcgcagcccaaggcctacgcttattggtaccagcagaagtctggacaggcccccatcttggtgatctacggtgattccaagcgcccctctggcatcccggagcgttttagtgggtcctcctcaggcacaatggcgacactgaccatttctggggcacaggtggaggatgaagcggactattactgttattctaccgaccgctcgggcaaccgcgtgttcggcgggggtacccgcctgactgtgctgactaccacccccgccccccgcccccctactccggccccaaccatcgcatcccagccgctgagtctgaggccagaggcttgtcggcctgccgctggcggcgctgtacacacccgaggattggatttcgcgtgcgacatctatatttgggctcctctagctggcacctgtggcgtgctgttgctctccctggtaatcaccctgtactgtaagcgtggccgcaagaagctgctatacatcttcaaacaaccgtttatgcgcccggtgcaaacaacccaggaggaggacgggtgctcatgccgcttcccagaagaagaggagggcgggtgcgagctacgcgtcaaattttcacggtctgcggacgcccctgcctacaagcagggccagaaccagctttacaacgagctcaaccttggtcgtcgcgaggagtacgatgtgctcgacaagcgaagagggcgggacccggagatgggcggcaagccacgtcgtaagaacccgcaggagggcctgtacaacgagctgcagaaggacaagatggccgaggcctactccgagatcggcatgaaaggcgagcgccgccgcggaaagggccacgacggcctctaccagggtctcagcactgcgaccaaagacacctacgacgctctgcatatgcaggccttgcccccgcgtggctccggtgcgactaacttctcgctccttaagcaggccggcgacgtggaggagaatcctgggcctatgggtgctggtgccaccggccgcgccatggatggcccccgtctgctgctattgctgttgctgggcgtgtccctgggtggcgccaaggaggcctgcccaaccggactgtacacccactctggagagtgttgcaaggcgtgtaacctgggcgagggcgtcgctcagccctgcggcgccaaccagacggtctgcgagccctgcctggactcggtgaccttctccgacgtggtatccgcaacggagccgtgcaagccctgcaccgagtgcgtgggcttgcagtccatgtcggccccttgcgttgaagccgacgacgcagtttgtaggtgtgcctacgggtattaccaggacgagactacgggcagatgcgaggcgtgcagggtttgtgaggccggcagcggtctggtatttagctgtcaggacaaacagaacacggtctgtgaggagtgcccagacggcacctactccgacgaggccaatcatgtggatccatgcctgccttgcactgtctgtgaagatactgagcggcaactgagggagtgcacccggtgggcggacgcagagtgcgaggagattcccgggcgctggattacccgctctacccctccggagggatccgattctactgcgccctccacccaggagccagaggctcctcccgaacaggatcttatcgccagcaccgtggcgggcgtggtgacaaccgtgatgggctcgtcgcaacccgtcgtcactcgcggcaccaccgacaacctgatccccgtgtactgttcaatcctggctgcggtggttgtcggcttagtggcttatatcgcgttcaaacggtggaattcctga

SEQ ID NO: 189 - protein, 788 aa, synthetic construct
MALPVTALLLPLALLLHAARPQVQLVQSGAEVKKPGASVKVSCKASGYTFSGYAMHWVRQAPGQGLEWMGWINPKSPGTNYAQKFQGRVTMTRDTSISTAYMELSRLRSDDTAVYYCARYSSSSWGDPWGQGTLVTVSSGGGGSGGGGSGGGGSGGGGSSYELTQPPSVSVSPGQTARVTCSGDAQPKAYAYWYQQKSGQAPILVIYGDSKRPSGIPERFSGSSSGTMATLTISGAQVEDEADYYCYSTDRSGNRVFGGGTRLTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 190 - DNA, 2367 bp, synthetic construct
  source 1..2367: mol_type = other DNA
atggcgctgccggtaaccgcgctactcctacccctggcactgttgttgcacgccgcacgacctagctacgagctgacgcagccgccctccgtgtccgtctcccctggacagacggcccgggtcacctgcagtggcgacgcacagcccaaggcctatgcgtattggtaccagcagaagtcgggacaggcccccatcctggtgatttacggtgactccaagagaccgtccggtatcccagagaggttttcaggtagctcctccgggaccatggctaccctgaccatctccggcgctcaagtcgaggacgaggcggactactactgttattcgaccgaccgctctggcaaccgcgtgttcggcgggggcacgcgactcactgttcttggcggaggtggttccggcggaggaggatcgggcggcggcggttctggcggcgggggctcacaggtgcagctcgttcagagcggcgctgaggtgaagaagcccggcgccagtgtcaaggtttcgtgcaaggcctctggctacaccttttccggttacgccatgcattgggtgcgccaggcccccgggcaaggcctggagtggatgggttggattaatcccaaatcccccggaaccaactacgcgcagaagttccagggacgcgtcaccatgacgcgcgacaccagcatctccacagcgtacatggagctgtctcgcctgcgctccgacgacactgcggtgtactactgtgcccgctacagctcatctagctggggcgacccgtggggccagggcactctggtgaccgtgagctccaccactacccctgcgcccagaccgcctacccccgccccaaccattgcttcccagccattgtctctgcgccctgaagcgtgccgtccggctgccggtggagcggtgcacacccgcggactcgacttcgcttgtgacatctacatctgggctccccttgctgggacctgtggagtgctgttactttctctcgtgataacactgtattgtaaacgtggccgtaagaagttgctgtatatcttcaaacagcccttcatgcgccccgtgcagaccacccaggaagaggatgggtgctcttgtcgcttcccggaggaggaggagggcgggtgtgagctgcgcgtcaaattttccaggtccgccgacgccccggcctacaagcagggtcagaaccagctgtacaacgagctcaacctgggccgccgcgaggagtacgacgtgctggacaagcgtcgcggtcgggaccctgagatggggggcaagccgcgccgcaagaaccctcaggagggcctgtacaacgagctgcagaaggacaaaatggccgaggcctactccgagattggaatgaagggagagcgccggcgtggcaagggccatgacggcctttatcagggcctgagcacggccaccaaggacacctacgatgccctccacatgcaggcgttacctccccgagggagcggtgctaccaatttcagtctgctgaagcaggccggcgatgtggaggagaacccgggccctatgggcgccggggctactgggcgtgctatggatggaccccgcttgctgctattgctgctgctgggggtgtcgttaggcggcgcgaaggaggcctgcccaaccggtctttacacccactcgggggagtgttgtaaagcatgtaacctgggcgagggtgtggcgcagccctgcggggccaaccaaacggtctgtgagccttgcctggacagcgtgaccttctcagatgtggtgtccgcaacagaaccatgcaagccatgcactgagtgcgtcggcctgcagtctatgtctgcgccttgcgtggaggctgatgacgccgtgtgccgctgcgcttacggctattaccaggatgagactaccggccgttgtgaggcgtgcagggtttgtgaggctggctccggtctggtattttcatgccaggacaaacagaacacggtgtgcgaggagtgcccagacggcacttactcggacgaagccaatcacgtggacccctgcctgccgtgcaccgtgtgcgaggacactgagcggcagcttcgcgaatgcactaggtgggcagatgctgagtgcgaggagatccccggccggtggatcacccgctccacaccccccgagggttctgatagcaccgctccctccacccaggagcccgaagcccccccggaacaggacctgatcgctagtaccgtggccggcgtggtgactacagtgatgggctcgagccaacctgtggtcacgcgcggtaccaccgacaacctcatcccggtgtactgctcaatcctggcagctgtcgtcgtagggctggtggcctacatcgcgttcaagcgttggaactcgtga

SEQ ID NO: 191 - protein, 788 aa, synthetic construct
MALPVTALLLPLALLLHAARPSYELTQPPSVSVSPGQTARVTCSGDAQPKAYAYWYQQKSGQAPILVIYGDSKRPSGIPERFSGSSSGTMATLTISGAQVEDEADYYCYSTDRSGNRVFGGGTRLTVLGGGGSGGGGSGGGGSGGGGSQVQLVQSGAEVKKPGASVKVSCKASGYTFSGYAMHWVRQAPGQGLEWMGWINPKSPGTNYAQKFQGRVTMTRDTSISTAYMELSRLRSDDTAVYYCARYSSSSWGDPWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 192 - DNA, 2373 bp, synthetic construct
  source 1..2373: mol_type = other DNA
atggccttgcctgtaaccgccctgttgctgcccttggcgctcctgctacacgccgccaggcccgaagtgcagctgcaacagtctggtcctgagcttgtgaagaccggggcgtccgtaaagataagctgcaaggcctctggttattctttcaccggctacttcatccactgggtcaagcagagccatggaaagtcactcgagtggattggatacatctcctgttacaatggcgcaaccagctacaaccagaagttcaagggcaaagcgactttcaccgtggacacatcctcctctactgcgtacatgcagttcaactccgtcaccagcgaggactccgccgtgtactactgcgtgcgcaactattacggcaacctggacgctatggactattggggccagggcacctctgtgacggtgagctctggcggcgggggctccggcgggggcggtagtggagggggaggctccggtggtggcggctcagacatcgtgatgacccagagccacaagtttatgtctacctccgtgggcgaccgcgtctccatcacctgcaaggcctctcaggacgtgtccaccgccgtcgcttggtaccaacagaagcccggacagtcgcctaagatcctgatttactctgcttcgtaccgctacaccggcgtgcccgacaggttcaccggttctggttctggcaccgactttacgttcaccattagttccgtgcaggccgaagatctggcggtgtactactgtcagcagcattacagtactccgcgcaccttcgggggtgggactaagctggagatcaagaccacgacacctgcgccgcgtccacccacccctgcccccaccatcgcctcccagcccctgtccctgagaccagaggcttgtaggccagcagcggggggggcagtgcacacccgcggcctggatttcgcttgtgacatctacatctgggcaccgctggcgggcacttgcggggtcctgcttctgtcgctggtgatcaccctgtattgtaaaagaggccgtaagaagctgctttatatcttcaaacagcccttcatgcgccccgtgcagactacccaggaggaggacggatgctcttgtcgcttccctgaggaagaggagggcggctgcgagctgcgggtgaaattttcccgctcggcggacgctcctgcttacaagcagggtcagaaccagctttacaacgagctcaacctcggacgacgtgaggagtacgacgtgctggataagcgccgtggccgtgacccggagatgggcggcaagcctcgccgcaagaaccctcaggaaggactctacaacgagctgcagaaggacaagatggctgaggcctactccgagatcggtatgaagggagagcggcgccggggtaaaggccacgatggactgtaccaaggcctgtctactgcaaccaaagacacttacgatgctctgcacatgcaggccctgccccctcggggcagcggagccaccaacttttcgctgctgaagcaggccggtgatgtggaggagaaccccgggccaatgggggcaggtgccacaggccgcgccatggacggcccacgcctcttgctgctgctcttgctaggtgtatctctgggcggggccaaggaggcgtgcccaaccggtctgtacacccactcaggcgagtgctgtaaggcttgtaatttaggcgagggtgtcgcgcagccgtgcggggcgaatcagacggtttgtgagccctgcctggacagcgtgacatttagcgatgtggtatcggccactgagccttgcaaaccgtgcacggagtgcgtgggcctgcaatccatgtccgctccgtgcgtggaagccgatgatgctgtttgtcgatgcgcctacggctattaccaggatgagaccaccggacgctgcgaggcctgccgggtgtgcgaggctggctcgggcctggtgttctcatgccaggacaaacagaacaccgtgtgcgaggagtgtcccgacggcacctactccgacgaggccaaccatgttgatccatgcctgccctgtaccgtctgtgaagacaccgagcgccagctgcgcgagtgcactcgctgggcggacgcagagtgcgaggagatcccgggccgctggatcacccgcagcactccgcctgaaggctccgacagtacggccccctccacccaggagccagaggctcccccggaacaggacctgatcgcttccaccgtggctggcgtggtcaccacagtgatgggcagcagccagcccgtggtcactcgtggcacgacggacaaccttatccccgtctactgttccattctcgcggccgtggtggttggattggtcgcctatattgcgttcaagcgttggaattcgtga

SEQ ID NO: 193 - protein, 790 aa, synthetic construct
MALPVTALLLPLALLLHAARPEVQLQQSGPELVKTGASVKISCKASGYSFTGYFIHWVKQSHGKSLEWIGYISCYNGATSYNQKFKGKATFTVDTSSSTAYMQFNSVTSEDSAVYYCVRNYYGNLDAMDYWGQGTSVTVSSGGGGSGGGGSGGGGSGGGGSDIVMTQSHKFMSTSVGDRVSITCKASQDVSTAVAWYQQKPGQSPKILIYSASYRYTGVPDRFTGSGSGTDFTFTISSVQAEDLAVYYCQQHYSTPRTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 194 - DNA, 2373 bp, synthetic construct
  source 1..2373: mol_type = other DNA
atggccctgcctgtgactgcattactgctgccactggccctgctgttgcacgccgccaggcccgacatcgtgatgacccagagccacaagtttatgtctactagcgtgggggacagggtgtctatcacctgcaaggcttcccaggatgtcagcactgctgtcgcgtggtaccaacagaagcccggccagtctccgaagattctgatctattcggcgagctaccgctacaccggcgtgcccgaccgcttcaccggctccggttcaggaaccgactttactttcacaatttcttcggtgcaggccgaggacctggcggtttactactgccagcagcactactccaccccgcgcaccttcggtggcggcaccaagctggagatcaagggaggcggaggctccggaggcggcggctctggtggtggcggctctggcgggggcggttccgaggtgcagctgcagcaatccggccctgagctggtgaagaccggcgcctccgtgaagatctcttgcaaggcctctggatactccttcaccggctatttcatccactgggtcaagcagagtcatggaaagtcgctggagtggattgggtatatctcatgctacaacggcgctacctcctataaccagaaattcaaaggtaaggcaacctttaccgtggacactagctcgtccaccgcctacatgcagttcaactcggtcacatccgaagactccgccgtgtattactgcgtgcgcaactactacggcaacctggatgccatggattattggggccagggtacctcggtaaccgtgtcctccacaacaacccccgccccgcgtccccctacccccgctccaaccatcgcttctcagccgctgagtcttcggcccgaggcgtgccgaccggctgctggcggcgcggtccacacgcgtggcctcgacttcgcctgtgacatctacatatgggcgcccctggccggcacctgcggggtgctgctgttgagcctggtgattaccctgtactgtaaacggggccgcaagaagctcctctacatcttcaagcagcccttcatgagacctgtgcagacgacccaggaggaggatgggtgctcttgtcgtttccccgaagaggaggaaggggggtgcgagctgcgcgtgaaattttcgcgcagcgccgacgcgcctgcctacaagcagggccagaaccaactttacaacgagctcaacctgggcagaagggaggagtacgacgtgcttgacaagcgccgtggtcgggatccagagatgggaggcaaaccgcggcgcaagaaccctcaggagggcctctacaacgagcttcagaaggacaaaatggctgaggcgtacagcgagatcggtatgaagggcgagcggcgccgcgggaagggacatgacggtctctaccagggtctgtccacggccaccaaggatacctacgatgctttgcacatgcaggccctgcccccgcgcggcagcggggccaccaacttttccctactgaagcaggccggggatgtggaggagaatccagggcccatgggcgcgggcgccactggacgcgccatggacggcccccgcctgctgctgctactacttttaggggtgtccttgggtggcgccaaggaggcgtgcccaaccggactgtacacgcatagcggtgagtgctgtaaggcctgtaacctcggggagggcgtcgcgcagccatgtggcgctaaccagactgtctgcgagccatgcttggattctgtcacctttagcgacgtggtgtctgcaactgagccatgtaaaccctgcacggagtgcgtgggactgcagtctatgtccgccccttgcgtggaggcagatgacgcagtttgtcgatgtgcctatggctactatcaagacgagaccaccggcaggtgtgaagcgtgccgcgtgtgcgaggctgggtccggattggtgttctcatgccaggacaaacagaacacggtgtgcgaggagtgtcctgacggcacctacagtgacgaggctaatcacgtggacccgtgcctgccctgtacggtctgtgaagacaccgagcgccagctgcgcgaatgcacacgttgggcggacgccgagtgcgaggagattcccggtcgctggatcacccgcagtactcctcctgaagggagtgacagtacagccccttccacccaggagcccgaggctccccccgaacaggatctgatcgcatctactgtcgctggtgtagtgacgaccgtgatgggtagctcgcagccggtcgtgactcgtgggaccacagacaacctcatcccggtgtactgttccatcctggctgctgtggttgtgggcctggtagcctacatcgcgttcaagcgttggaattcctga

SEQ ID NO: 195 - protein, 790 aa, synthetic construct
MALPVTALLLPLALLLHAARPDIVMTQSHKFMSTSVGDRVSITCKASQDVSTAVAWYQQKPGQSPKILIYSASYRYTGVPDRFTGSGSGTDFTFTISSVQAEDLAVYYCQQHYSTPRTFGGGTKLEIKGGGGSGGGGSGGGGSGGGGSEVQLQQSGPELVKTGASVKISCKASGYSFTGYFIHWVKQSHGKSLEWIGYISCYNGATSYNQKFKGKATFTVDTSSSTAYMQFNSVTSEDSAVYYCVRNYYGNLDAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 196 - DNA, 2370 bp, synthetic construct
  source 1..2370: mol_type = other DNA
atggctctgccggtgactgctctgctgctgcctttggcgctgctgcttcatgccgctaggcccgaggtgcagctggtggagagcggtggcgatctcgtgcagccgggggggtcgctcaagctttcgtgtacagcatctggcttcactttttccaactacggcatgtcctggattcggcagacgcccgacaagcggctggagtgggtggcaaccatcgtgggcaacgactacacctacttcccggactccatgaaaggccgtttcaccgtgtctcgagataacgcgaagtctatcctgtacctgcagatgaacagcctggcctccgccgacacggcgatgtattactgcactcgtcatgactgggtcttcgactattggggccagggtactccgctgaccgtgagctccggtggaggtggctctggaggcggagggtccggaggtggcgggtccggcggcggcggcagtgacattgtcctcacccagagccccgcttctctcgccgtgtccctgggtcaacgcgccatcctgtcatgcaaggcatcacagagtgtgagcttcgccggctcctcgctgatgcactggtaccaacagaagcctggacagcagcccaagctcctgatttaccgcgcctccgacctggagactggcatcccaaccaggttcagtggtggcggttctggcaccgactttaccctaaacatccaccccgtagaggaagatgacgcagccacctattactgccaacagtccagggagtacccctataccttcggcggcggcacccgcttagaaattaagaccacaacccccgcgccccggcccccaaccccagcgccgaccattgctagccagccactgtccctgcgccctgaggcgtgccgaccagcagctggaggcgccgtccacactcgcggtttggacttcgcgtgtgacatctacatatgggcccccctggccggcacctgcggggtgctgctgttgtctctggtcatcaccctgtactgtaagcgcggccgcaagaagctgttatacatcttcaagcaacccttcatgcgtccggtgcagacaacccaggaagaggacgggtgctcttgcagatttcctgaagaggaggagggagggtgcgagctacgcgtgaaatttagccgctccgccgacgcgccggcctacaagcagggtcagaaccagctgtacaacgagctgaacctgggccgccgcgaggagtacgacgtgttggataagcgccgtggacgcgatccggagatggggggcaaaccgcgccgtaaaaaccctcaggagggcctctacaacgagcttcagaaggacaagatggccgaggcctactccgagatcggtatgaagggcgagcggcgccgtggcaaaggccacgacggcctctaccagggcctgagcacggcaaccaaggatacctacgatgctctgcacatgcaggccctgcccccgcgtggctccggtgcgaccaacttttctctgctgaagcaggctggtgatgtggaagagaatcctggccctatgggcgctggggctaccggaagagccatggatggcccccgcttgctgcttctcctgctcttgggtgtcagtctgggcggagccaaggaggcgtgcccaacggggctttatacacactccggggaatgttgtaaggcctgcaatctgggtgagggcgtggcccagccatgcggggccaaccagactgtctgtgaaccatgtctggattccgtgaccttcagcgacgtggtgtcggctacggagccctgcaagccctgcaccgagtgcgtgggacttcagtcgatgtctgctccctgcgtggaggccgatgacgccgtgtgccgctgcgcctacggctactaccaggacgagactaccggccgctgtgaagcctgtcgcgtctgtgaggctggctccggtttggtgttctcatgccaggataaacagaacacggtttgcgaggagtgtcccgacggaacttactcagacgaggccaatcacgttgacccgtgcctgccgtgcaccgtctgtgaggacaccgagcgccagctgagggagtgcacccgctgggcggacgctgagtgcgaggagatccccgggcgctggatcactcgctcgacccctcccgagggctctgacagcactgctcccagtacccaggagcccgaggctcctcctgaacaggaccttatcgcctccacagtagccggcgtggtcaccaccgtgatgggctcgtcccagcctgtggtgacccgcggcacgactgacaacctgatccctgtatactgtagcatcctggcggccgtggtggtagggctagtggcgtatatcgcgttcaaacgatggaattcctga

SEQ ID NO: 197 - protein, 789 aa, synthetic construct
MALPVTALLLPLALLLHAARPEVQLVESGGDLVQPGGSLKLSCTASGFTFSNYGMSWIRQTPDKRLEWVATIVGNDYTYFPDSMKGRFTVSRDNAKSILYLQMNSLASADTAMYYCTRHDWVFDYWGQGTPLTVSSGGGGSGGGGSGGGGSGGGGSDIVLTQSPASLAVSLGQRAILSCKASQSVSFAGSSLMHWYQQKPGQQPKLLIYRASDLETGIPTRFSGGGSGTDFTLNIHPVEEDDAATYYCQQSREYPYTFGGGTRLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 198 - DNA, 2370 bp, synthetic construct
  source 1..2370: mol_type = other DNA
atggcgctgcctgtcacggccttgctactccccctggctctgcttctgcacgccgcccgtcccgacatcgtactgacccagagcccagcgagccttgcggtgagcctggggcagcgcgccatcctgtcttgtaaggcttctcagtccgtgtccttcgcgggttccagcctgatgcactggtaccagcagaagcctggtcagcagcccaaattgctgatctaccgcgcttccgacctggagactggcatccctacccgtttttccggaggaggctccggcaccgacttcacgctcaacatccaccccgttgaggaagatgacgcggctacttactactgtcaacagagccgcgagtacccctataccttcggcggaggaacgaggttggagatcaagggcggcggagggtcaggaggcggaggctctggcggcggtggaagcgggggtggcggctccgaggtgcagctggtagagagcggcggggacctggtgcagcctggtggctccttaaagctttcttgcaccgcttccggcttcacctttagcaactacggtatgagttggattcgtcagaccccggataagcgcctggagtgggtcgcaacaattgtcggcaacgactacacctacttcccagattctatgaaggggcgcttcaccgtgtcgcgcgacaacgcgaagagcatcctgtacctgcagatgaactccttggcctctgctgataccgccatgtattactgtacccgccatgactgggtgttcgactattggggccagggcacccctttgacagtgtcgtccactacaacccccgctcctaggccaccaaccccagccccaaccatcgcctcccagccactctcgctgcgccccgaggcttgccggccggcggccgggggggctgtccacacccgtggattggacttcgcatgcgacatttatatttgggcacccctggctgggacctgcggtgtactgctgttgtccctggtgataaccctgtactgtaagcgcggccgaaagaagctgctgtatatcttcaaacagcccttcatgcgtcctgtgcagacaacccaggaggaagacgggtgctcgtgccggtttcctgaggaggaggagggtgggtgcgagctgcgcgtgaaattttcccgttccgctgacgccccagcctacaagcaaggacagaaccagctctacaacgagcttaacctgggccgcagagaggagtacgacgtgctcgacaaacgccgcggtcgtgacccggagatgggcggcaagccccgccggaagaatcctcaggagggcctatacaacgagttgcagaaggacaagatggctgaagcatactcggagattggaatgaaaggcgagcggcgccggggcaagggccacgacggcttatatcagggtctttcgacggcaacgaaagacacctacgatgctcttcatatgcaggccctgccgccgcgcggttccggagctaccaacttttcattgctgaagcaggccggagatgtggaggagaatcccggccctatgggggccggggccaccggtagagccatggatggcccccgactgctgctcctccttttactaggcgtttctctgggcggcgccaaggaggcctgcccaaccggtctgtacacccactccggtgagtgttgtaaggcctgcaacctgggtgagggcgtggcgcagccctgcggggccaaccagactgtctgtgaaccgtgcctggattctgtgactttcagcgatgtggttagtgcaaccgagccttgcaaaccctgtactgagtgcgtgggcctgcagtccatgtctgctccgtgcgtggaagcggatgacgccgtctgtaggtgtgcctacggctactaccaagacgagaccacaggcaggtgtgaggcttgtcgagtgtgcgaggctggttcgggccttgtcttctcatgccaggacaagcagaatacggtctgtgaggagtgtcccgacggcacttactccgacgaggccaaccacgtggatccttgcctgccctgcactgtttgcgaagacaccgagcgccagctgcgcgagtgtacccgctgggcagacgccgagtgcgaggagatccccggccgctggatcactcgctcgactccgcctgaaggcagtgacagcactgctccctctacgcaggagcccgaggctcccccggaacaggacctcatcgcctctaccgtggcgggcgtcgttaccaccgtgatgggctccagccagccggtggtcaccagaggcactacagacaacctgatcccggtgtactgctctatcctggcggcggtggtggtgggcctcgtggcctacatcgcgttcaagcggtggaattcgtga

SEQ ID NO: 199 - protein, 789 aa, synthetic construct
MALPVTALLLPLALLLHAARPDIVLTQSPASLAVSLGQRAILSCKASQSVSFAGSSLMHWYQQKPGQQPKLLIYRASDLETGIPTRFSGGGSGTDFTLNIHPVEEDDAATYYCQQSREYPYTFGGGTRLEIKGGGGSGGGGSGGGGSGGGGSEVQLVESGGDLVQPGGSLKLSCTASGFTFSNYGMSWIRQTPDKRLEWVATIVGNDYTYFPDSMKGRFTVSRDNAKSILYLQMNSLASADTAMYYCTRHDWVFDYWGQGTPLTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 200 - DNA, 2367 bp, synthetic construct
  source 1..2367: mol_type = other DNA
atggcgttgccagtgactgctctgctgctgcccctagctctcctcctgcacgccgcgaggcctcaggtccagctggtggagagcggagcggaggtgaaaaagccaggcgcctccgtgaaggtgtcctgtaaggcctccggctacaccttcaccaactacatcatccactgggtcaagcaggagcccggccagggcctggagtggattggctatttcaacccctataaccatggaaccaagtacaacgagaaatttaagggcagagccactctgacagccgataagagcatctctaccgcgtacatggagctgtcctccctacggagcgaggatacggccgtatactactgcgctcgctctggaccctatgcgtggtttgacacctggggccagggcacgaccgtgactgtatcgtccgggggcggcgggagtggcggcggtggctccggcggtggcggcagcggcggagggggttccgacatccttctgacccagagccctgcaaccctgagcctgtcgcctggggagcgggcaactttgtcctgtcgagcctctcagaacatcggtacttccatccagtggtaccagcagaagcctggacaggccccgcgcctcctcatccgcagctcctccgagagcatttctggtatctcttcccgcttcagcggatcgggatctgggacggacttcacgttgacaatcagttcgctggaacctgaggactttgcagtgtactactgtcaacagtcaaatacctggccgttcacctttggccagggcaccaagctggagatcaagaccactacccctgccccccgcccgcctacccccgcgccgaccatcgcctctcagcccctgtctctgcgccctgaagcttgtcgccccgctgcaggcggagccgtgcacacacggggtcttgatttcgcatgtgacatctatatatgggcccccctggctggcacctgcggggtgctgctattgtccctagtcatcaccctttattgcaagcgcggccgcaagaagctcctgtacatcttcaaacagcccttcatgcgccccgtgcagacgacccaggaggaggacgggtgcagctgtaggttccccgaagaagaggagggtgggtgcgagcttcgcgttaaattctcccgtagcgcggacgctcctgcctacaagcaaggtcagaaccagttgtacaacgagctcaacctgggccgccgcgaggagtacgatgtcctcgacaaacgtcgtggtagagacccggagatggggggcaagccgcgtcgaaagaaccctcaggagggcctttacaacgagctccagaaggacaagatggctgaggcttactctgagatcggaatgaaaggcgagcggcgtcgcggtaagggccatgacggcctgtaccaaggccttagtactgccaccaaagacacctacgacgcgctgcacatgcaggccctgcctccgcgtggttctggcgccacgaacttttcattactgaagcaggctggggacgtggaggagaatccaggccctatgggcgcgggtgccactggtcgcgccatggatggcccccgcctgcttctgctgttactgcttggcgtgtccttagggggcgctaaggaggcgtgcccaaccggtctgtacacccactccggagaatgctgtaaagcctgcaatctgggcgagggcgtggcgcagccatgcggggcgaaccagaccgtatgcgagccatgcctggatagtgtgaccttctcagacgtggtgtccgccaccgagccgtgcaagccgtgcactgagtgcgtgggtctgcagtctatgtctgctccctgcgtggaggcagatgacgctgtctgtaggtgtgcctacggctactaccaagacgagaccaccggcagatgtgaggcttgtcgcgtgtgcgaggctgggtcgggcttggtgttctcatgccaggataagcagaacacggtctgcgaggagtgtccggacggcacttactccgacgaggccaaccacgtggatccatgcctgccctgcactgtctgtgaagacaccgagcgccagctccgggagtgcacacgctgggcagacgccgagtgcgaggagatccccggccgctggataacccgctcgactccgcctgaaggtagcgattccacagctccctccacccaggagcccgaggccccccccgaacaggacctgattgcttccaccgtggcgggcgttgtgaccaccgtgatgggcagtagccagccggtggtcacccgcggaacaaccgacaacctgattccggtttactgttctatcctggccgccgttgtggtgggcctggtcgcgtatattgcgttcaagcgttggaattcctga

SEQ ID NO: 201 - protein, 788 aa, synthetic construct
MALPVTALLLPLALLLHAARPQVQLVESGAEVKKPGASVKVSCKASGYTFTNYIIHWVKQEPGQGLEWIGYFNPYNHGTKYNEKFKGRATLTADKSISTAYMELSSLRSEDTAVYYCARSGPYAWFDTWGQGTTVTVSSGGGGSGGGGSGGGGSGGGGSDILLTQSPATLSLSPGERATLSCRASQNIGTSIQWYQQKPGQAPRLLIRSSSESISGISSRFSGSGSGTDFTLTISSLEPEDFAVYYCQQSNTWPFTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 202 - DNA, 2367 bp, synthetic construct
  source 1..2367: mol_type = other DNA
atggccctgccagtaactgcacttctgctacctttggctctgctcctgcacgccgctcgccctgacatcttgctcacccagagccctgcaactctaagcctctcaccgggcgaaagggccactctttcctgtcgggcgtcacagaacatcggcaccagcatccagtggtaccagcagaagcccggccaggcccccaggctgctcattcgcagtagttccgaaagcatctctggcatctcgtcccgcttctccggcagcgggtcgggtaccgatttcaccctgaccatctcatcgctcgagccagaagactttgctgtctactactgccaacagtcaaatacctggccgttcaccttcggccagggcaccaagctggagattaaaggtggcggtggctccggtggaggcggctcgggaggcggcggctccgggggtggcggatcccaggtccaactggtggagagcggcgcggaggtgaagaagcctggggcttccgtgaaggtgtcttgtaaggcttccggctacacgttcacgaactacatcatccactgggtgaagcaggagcccgggcagggactcgagtggattggctatttcaacccctataatcacggcaccaagtacaacgagaagtttaaggggcgcgctacactcactgcggacaagagcatcagcaccgcttacatggagttgtccagtctgcgctcggaagatacagccgtgtactattgcgctcgctctgggccgtacgcgtggttcgacacctggggccagggcacgaccgtcaccgtgtcctctaccacgaccccggctccgcgccctcccactcccgctcctaccattgcttcccaacccctgagtttgagacctgaggcttgccgtcctgcagcaggcggagccgtccacacacgtggcctggacttcgcctgtgacatctacatctgggcgcccctggccggaacctgcggggtactgctcctgagcctggtgattacgctgtactgtaaacgcggtcgaaagaaactgctttatatctttaaacagcccttcatgcgtcccgtgcagacgacccaggaggaggatggctgttcctgccgtttccccgaggaagaggagggcggttgcgagctgagagttaaattttcgcgcagcgcggacgcccccgcctacaagcagggtcagaatcagctgtacaacgagctcaacctgggtcgccgcgaggagtacgacgtgttggataaaaggcgcggccgcgacccggagatgggcggcaagccgagacgcaagaaccctcaggagggcctttacaacgagctgcagaaggacaagatggctgaggcctactccgagataggtatgaagggcgagcgtcgccgtggaaagggtcatgacggcctatatcagggcctgtccaccgccacgaaggacacttacgacgcgctgcacatgcaggctcttcctccccgcggtagtggggccaccaatttttccttgctgaagcaggccggtgatgtggaggagaacccagggcccatgggtgctggcgccaccggccgggccatggatgggccgcgcctgctgctgctgcttctgctgggcgtgtcgctgggcggcgccaaggaggcgtgcccaaccggtctttacacccattctggcgagtgttgtaaggcctgcaacctgggcgagggcgtggcgcagccgtgcggggccaaccagacagtctgtgagccgtgcctagacagcgttactttcagcgatgtggtgagcgcaaccgaaccatgtaagccctgcactgaatgcgtggggttgcagtctatgtctgctccttgcgtggaggccgatgacgcagtgtgccgctgtgcctacggctactaccaggacgagaccacaggacgatgcgaggcctgtcgggtctgtgaggctggctccgggctggtattctcctgccaggacaaacagaacacggtttgtgaggagtgccccgacggcacctactctgacgaggccaaccacgtagatccatgcctgccctgcactgtctgtgaggacacggagcgccagctgagggagtgtacccgctgggcggacgccgagtgcgaggagatccccggccgttggatcacccgctctactcctcctgagggatctgactccactgccccctctactcaggagccagaggctccccccgaacaggacctgattgcctcgaccgtggcgggcgtggtgaccacagtgatgggatcttcacagccggtggtcactcgcggaaccaccgacaaccttatcccggtttactgctctatcctggcggccgtggtggtgggcttggtcgcctatatcgcgttcaagcgttggaactcctga

SEQ ID NO: 203 - protein, 788 aa, synthetic construct
MALPVTALLLPLALLLHAARPDILLTQSPATLSLSPGERATLSCRASQNIGTSIQWYQQKPGQAPRLLIRSSSESISGISSRFSGSGSGTDFTLTISSLEPEDFAVYYCQQSNTWPFTFGQGTKLEIKGGGGSGGGGSGGGGSGGGGSQVQLVESGAEVKKPGASVKVSCKASGYTFTNYIIHWVKQEPGQGLEWIGYFNPYNHGTKYNEKFKGRATLTADKSISTAYMELSSLRSEDTAVYYCARSGPYAWFDTWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 204 - DNA, 2355 bp, synthetic construct
  source 1..2355: mol_type = other DNA
atggctctcccggtcaccgctctgctgttgcccctggcgttgttactccacgccgcgaggcctcaaatcactctaaaggagagcggacctggcatcctgcagccgtcacaaactctttccttaacctgctccttctcgggcttcagcttgactacctacggcatcggtgtgggctggatccgccagccacccggcaagggcctggagtggctcacccacatctggtggaacgacaacaagtactacaacactgcgctccgctctcgcctgaccatcagcaaagactccagcaacaaccaggtcctgctgaagatcgccaacgtggataccgccgacacagccacttactactgcctgtacggctacacctattggggccaggggaccctggtaacggtgtccgccggcggcggggggtccggaggcggtggctctggcggtggtggcagtgacgtggtgatgacccagactccgctaagcctgccagtatccctgggggaccaggcgtccatatcgtgccgatctagccagagccttctttactccaatggcaacacgtacctgcactggtacctgcagaagcccggtcagagccctaagctgctcatttacaagttgtccaaccgcttttctggggtgcccgacaggttttctggctccggatccggcaccgacttcaccctgaagatttctcgagtggaggcggaggacctgggcgtctatttctgttcgcagtctacccacgttccctggaccttcggaggcggcaccaagctggagatcaagaccacaacacccgcccctcgcccccctaccccagccccgacaattgcttcccagccactgagtttgcgccccgaggcttgccgtcctgccgcgggtggcgcggtgcacactcgcggcttggatttcgcttgtgacatctatatatgggctcccctggccggcacctgtggagttcttttgttatccctggtgatcactctctactgcaagcgcggacgcaagaagctgttgtacatcttcaagcagcccttcatgaggccggtgcagacgacccaggaggaagacgggtgctcatgccgcttccccgaggaggaggaaggcgggtgcgagctgcgcgtgaaattttcccgcagcgcggacgcccctgcgtacaagcaaggacagaaccagctgtacaacgagctcaacctgggccgcagggaggagtacgatgtgctggacaagcgccgtggtcgcgacccggagatgggggggaagccgcgccgcaagaatcctcaggagggcctttacaacgagctgcagaaggacaaaatggcggaggcctactccgagattggaatgaaaggcgaacggcgtcgtggcaaaggacatgacgggctgtaccagggcctatcgaccgcaacaaaggacacctacgacgcactacacatgcaggccctgcccccgcgcggctctggcgccaccaacttttccctgctgaagcaggctggcgatgtggaagagaaccccgggccgatgggcgctggagctaccggccgcgccatggatggtccccgtctgctgctgctgctccttctaggcgtgtctctgggtggtgctaaggaggcttgccctaccggtctgtatacgcacagcggagagtgttgcaaggcctgcaacctgggggagggggtggcgcagccgtgcggggctaaccagactgtctgtgaaccatgtttggattctgtgaccttcagtgacgtggtgtccgcaactgagccctgcaagccctgtacggagtgcgtcggccttcagtccatgtctgcgccctgcgtggaggcagatgacgctgtctgtaggtgtgcctacggctattaccaggacgagaccaccggccgttgtgaggcctgccgggtctgcgaggcaggctcggggctcgtgttctcatgccaggacaaacagaacacggtctgcgaggagtgcccagacggtacctacagcgatgaagccaaccatgtagatccttgcctgccctgtacagtttgcgaggacaccgagcggcagctgcgcgagtgtacccggtgggcggacgccgagtgtgaggagatcccgggtcgatggatcacccgctcgaccccgcccgagggctccgacagcacggccccctctacccaggagccagaagctcctcctgaacaggatctgattgccagtactgtggccggcgtggtcaccaccgtgatgggctcctcccagccagtggtcactagaggtaccaccgataatcttatccccgtgtactgttctatcctggcagccgtggtggttggcctggtggcttatatcgcgttcaaacgctggaattcctga

SEQ ID NO: 205 - protein, 784 aa, synthetic construct
MALPVTALLLPLALLLHAARPQITLKESGPGILQPSQTLSLTCSFSGFSLTTYGIGVGWIRQPPGKGLEWLTHIWWNDNKYYNTALRSRLTISKDSSNNQVLLKIANVDTADTATYYCLYGYTYWGQGTLVTVSAGGGGSGGGGSGGGGSDVVMTQTPLSLPVSLGDQASISCRSSQSLLYSNGNTYLHWYLQKPGQSPKLLIYKLSNRFSGVPDRFSGSGSGTDFTLKISRVEAEDLGVYFCSQSTHVPWTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 206 - DNA, 2355 bp, synthetic construct
  source 1..2355: mol_type = other DNA
atggccctgcctgtgacagccctgctgctgcctctggctctgctgctgcatgccgctagacccgacgtggtgatgacccagacccccctgagcctgcccgtgagcctgggcgaccaggccagcatcagctgcaggagcagccagagcctgctgtacagcaacggcaacacctacctgcactggtacctgcagaagcccggccagagccccaagctgctgatctacaagctgagcaacaggttcagcggcgtgcccgacaggttcagcggcagcggcagcggcaccgacttcaccctgaagatcagcagggtggaggccgaggacctgggcgtgtacttctgcagccagagcacccacgtgccctggaccttcggcggcggcaccaagctggagatcaagggtggcggtggctcgggcggtggtgggtcgggtggcggcggatctcagatcaccctgaaggagagcggccccggcatcctgcagcccagccagaccctgagcctgacctgcagcttcagcggcttcagcctgaccacctacggcatcggcgtgggctggatcaggcagccccccggcaagggcctggagtggctgacccacatctggtggaacgacaacaagtactacaacaccgccctgaggagcaggctgaccatcagcaaggacagcagcaacaaccaggtgctgctgaagatcgccaacgtggacaccgccgacaccgccacctactactgcctgtacggctacacctactggggccagggcaccctggtgaccgtgagcgccacgacaaccccggccccgcgtcctcctacccccgcccccaccattgcaagccagccactgtctctgcgcccggaggcgtgccgacccgctgcaggtggtgccgtgcacactcgcggcctcgatttcgcttgcgacatctacatctgggctccattggccgggacctgcggggtattgctgctgtccctggtgatcactctttactgcaagcgcggccggaaaaagctgctctacatcttcaagcaacccttcatgaggcctgtccagactacccaggaggaagacggctgttcttgccgcttccctgaagaggaggaaggtggctgcgagctgcgggtgaaattttcccgcagtgcggacgcccccgcctacaagcagggccagaaccagctgtacaacgagctcaacctgggccgccgcgaggagtacgacgtgctggacaaacggcgcggccgtgatccggagatgggtggcaagcctcgtagaaagaatcctcaggagggcctgtacaacgagctgcagaaggataaaatggccgaggcgtactcggagatcgggatgaaaggcgagcgccgtaggggaaagggccatgacggtctctatcagggattgtctaccgccacgaaggacacctatgacgcactgcatatgcaggccctcccgccccggggttccggcgccaccaacttttcactgctgaagcaggctggtgacgtggaggagaatccaggccccatgggggccggcgccaccgggcgcgccatggatggaccccggctgttattactgctgctgcttggcgtgtccctaggcggggccaaggaggcttgtcctaccgggctctacacccactcgggggaatgttgtaaggcctgtaacctgggcgagggcgtggcgcagccgtgcggggcgaaccagacggtctgtgagccatgcttggacagtgtgaccttctctgatgtggtgtccgccaccgagccttgtaagccgtgcacagagtgcgtgggcctgcaatccatgagcgcgccgtgcgtggaggcagatgacgctgtctgccgctgtgcttacggctattaccaggatgagactaccggcaggtgtgaggcttgccgcgtatgtgaggcgggctcgggccttgtattctcttgccaggacaagcagaacacggtgtgcgaggagtgtcccgacggcacctacagtgacgaggccaaccacgtggacccctgcctgccgtgcaccgtgtgcgaggacaccgagcgacagttgcgcgagtgcactcgttgggctgacgcggaatgtgaggagatccccggtcgctggatcacccgctccacccctcctgaaggatccgattcgactgccccctctacccaggagcccgaggctccccctgagcaggacctgattgcttcgactgtcgctggcgttgtcaccacagtcatgggctcatcgcagccggtcgtcaccaggggcacgaccgacaacctgatacccgtgtactgctctatcctggcagccgtggtggtcggcctggtggcttacatcgcgttcaaacgctggaactcctga

SEQ ID NO: 207 - protein, 784 aa, synthetic construct
MALPVTALLLPLALLLHAARPDVVMTQTPLSLPVSLGDQASISCRSSQSLLYSNGNTYLHWYLQKPGQSPKLLIYKLSNRFSGVPDRFSGSGSGTDFTLKISRVEAEDLGVYFCSQSTHVPWTFGGGTKLEIKGGGGSGGGGSGGGGSQITLKESGPGILQPSQTLSLTCSFSGFSLTTYGIGVGWIRQPPGKGLEWLTHIWWNDNKYYNTALRSRLTISKDSSNNQVLLKIANVDTADTATYYCLYGYTYWGQGTLVTVSATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 208 - DNA, 2355 bp, synthetic construct
  source 1..2355: mol_type = other DNA
atggctctgccagtgacggcactcttgcttcccctggctctgttgttacacgccgcacgtccccaaatcacactcaaggagagcggccccaccctggtcaagcccacccagaccctcaccctgacctgtacgttctctggcttctccttgacaacctacggcatcggcgtgggctggatccgccagccacccggcaaggctctggagtggctcacgcacatctggtggaacgacaacaagtactactccccgagtctcaaaagccgtcttaccatcaccaaggattcctctaaaaaccaggtggtattgaccatgacaaatatggacccggtagacaccgccacttattactgtctgtacggctacacctattggggccaggggacactggtcaccgtcagtgcaggagggggaggtagcggaggcggtggctccggaggcggtggtagcgatgtggtaatgacccagactcctttgtcgctgcccgttaccctgggtcagccagcgagcatctcatgtaggagctcccagtccctgctatacagcaacggcaacacctacctgcattggtaccaacagcgtcccgggcagtccccccggctgcttatctacaagctcagcaaccgcttttctggcgttcctgaccgcttctccggatctggctccggcaccgacttcaccctgaagatttcacgcgtggaggcggaggacgtgggcgtgtactactgttctcaatccacccacgtcccgtggaccttcggcggcggcaccaagctggagattaagaccaccacgcctgccccgcgccccccaacgccggcccccaccatcgcctcccagccgctttccctgcgtcctgaagcgtgccgtcccgcggcggggggtgctgtccacactcgcggcctagactttgcctgcgacatctacatctgggcccctctagctggaacctgtggggttctgctgctgtcgctggtgattactctctactgtaaacgcggtcgcaagaagctgctctatatcttcaaacagcccttcatgcgacccgtgcaaacaacccaggaggaggacggctgttcatgccgatttcctgaggaagaggaaggtgggtgcgagttgcgcgtgaaattttcccggagcgcagacgcacccgcctacaagcagggccagaaccagctgtataacgagctcaacctcggacggcgcgaggagtacgacgtgctggacaagcgccgcgggcgagacccggagatgggcggaaaacccagaaggaagaatcctcaggagggtctgtacaacgagctgcagaaggacaagatggccgaggcttactccgagatcggtatgaagggtgagagacgcaggggtaagggccatgacggcctgtatcaggggctatcgaccgctacaaaggacacctatgatgcgcttcacatgcaggcgctgccaccccgcggctcgggggccaccaacttctcgctgctgaagcaggccggtgatgtggaggagaacccaggccccatgggcgccggcgctactggtcgcgccatggatggccctcgcttactgctgctgcttctgttgggcgtgtcgcttggtggcgccaaggaggcgtgccctaccggactgtacacccactccggcgaatgctgcaaggcttgcaatctgggagagggcgtggcccagccgtgcggggccaaccagacggtatgcgagccatgcctggactccgtgactttctccgacgtggtgagcgccaccgagccctgtaaaccctgcacggagtgcgtgggccttcagtctatgtctgcaccttgcgtggaagctgatgacgcggtgtgccgctgtgcctacggctactaccaggatgagaccactggccgctgtgaggcctgccgagtgtgcgaggccggctctggcctcgtgttcagttgccaggataagcagaacacagtctgtgaggagtgcccggacggcacttacagcgacgaggccaatcacgtcgatccatgtctgccctgcaccgtgtgcgaggatactgagcgccagctgcgcgagtgcacccggtgggcggacgcggaatgtgaggagatccctggtcgttggataacgcgctctaccccgccggagggcagtgactctactgctccgtctacccaggaacccgaggctccccctgagcaggacctgattgcttccactgtggctggggtcgtgacgaccgtgatggggtcctcgcagccggtggtcactcggggaacaaccgacaacctgattcccgtgtattgctctatcctggccgccgtggtggtcggacttgtggcgtacatcgcgttcaagcgttggaactcgtga

SEQ ID NO: 209 - protein, 784 aa, synthetic construct
MALPVTALLLPLALLLHAARPQITLKESGPTLVKPTQTLTLTCTFSGFSLTTYGIGVGWIRQPPGKALEWLTHIWWNDNKYYSPSLKSRLTITKDSSKNQVVLTMTNMDPVDTATYYCLYGYTYWGQGTLVTVSAGGGGSGGGGSGGGGSDVVMTQTPLSLPVTLGQPASISCRSSQSLLYSNGNTYLHWYQQRPGQSPRLLIYKLSNRFSGVPDRFSGSGSGTDFTLKISRVEAEDVGVYYCSQSTHVPWTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 210 - DNA, 2355 bp, synthetic construct
  source 1..2355: mol_type = other DNA
atggctctccccgttactgcactgctgttacctttggcgctccttctgcacgccgctcgccccgacgtggtgatgacccagactcccctgagcctgccggtgaccttgggtcagccagcttccatctcatgccgctcgtcacagagccttctgtactccaatggcaacacttacctgcattggtaccagcagcgccctggacagagcccgcgcctcctgatctacaagttgtccaaccgcttttctggagtgccagaccgcttctcgggctcgggctccggtactgacttcaccctgaagatctctcgcgtggaggcagaggacgtgggcgtctactactgtagccagagcactcacgtcccgtggaccttcggaggcggtacaaagctggagatcaagggcggtggtgggtctggaggcggcggctctggcggtggcggcagccaaattactctgaaggagagcgggccgaccctagtgaagcccacccagaccctgacgctaacctgcacgttcagcggcttctctctaacgacttacggcatcggtgtgggctggatccgccagccgcctggtaaggctttggaatggctcactcacatctggtggaacgacaacaagtactacagtccatctctgaagtcccgcctgaccatcaccaaggatagttccaagaaccaggtcgtactgacaatgacaaacatggatccagtggacaccgccacctattactgcctgtatggctatacctattggggccagggcactctggtgaccgtctccgccacgactacccctgctcccaggcctcctactcccgcgccgaccatcgcaagtcagccactgtcccttcgccccgaagcctgcagacccgccgcgggaggtgcggtgcacactaggggcctcgactttgcgtgcgacatctatatatgggctcctctggccggcacctgcggggtgctgctcttgtcactggtgattaccctttactgtaagcgcggccgcaagaagctgctctacatcttcaagcaacccttcatgcgtccggtgcagacgacccaggaggaagatgggtgcagttgtcgtttccccgaagaggaggaaggagggtgcgagctccgtgttaaattttccagatctgcggacgccccggcctacaagcagggacagaaccaactgtacaacgagctgaatctgggccgaagggaggagtacgatgtattggacaaacggcgcgggcgcgacccggagatgggcggcaagccccgccgcaaaaaccctcaggagggcctctacaacgagctgcagaaagacaagatggccgaggcctactccgagattggaatgaagggagagcggcgtcgcggcaaaggccacgacggtttatatcagggcctttcaacggcgaccaaggacacttacgacgcattacacatgcaggccctgcccccccgtggtagtggcgctaccaacttttccctgctgaagcaggccggtgatgtggaggagaatccaggccctatgggagcaggtgctaccggccgcgccatggatggcccgcgccttttgctactgctgctgctgggtgtatccctgggtggtgcgaaggaggcttgcccaaccggtctgtacacccattcgggggaatgttgcaaagcgtgtaacctgggggagggcgtggcgcagccctgcggggccaaccagaccgtgtgcgagccttgtctggactccgtcacattcagcgatgtggtgtcggctaccgagccctgcaagccttgcaccgagtgcgtgggactccagtctatgtctgcaccctgcgtggaggccgatgacgctgtctgtcgatgcgcctacggctactaccaggacgagaccaccggcaggtgtgaggcttgccgagtgtgcgaggcggggagcggcttggtgttctcctgtcaggataaacagaacacggtctgtgaggagtgcccagacggcacctacagcgatgaggctaatcacgtagacccgtgcctgccttgtacagtctgcgaagacaccgagcgccagctgcgcgagtgtacacggtgggccgacgccgagtgcgaggagatccctggccggtggatcacccgctccaccccccccgagggctccgactcgaccgctccctcgacccaggagccagaggctcccccggaacaggacctgattgcctccaccgtcgctggggttgtgacgaccgtgatgggctcctcccagcccgtggtcacccgtggcaccaccgacaacctcatcccggtgtactgttctatcctggcggccgtggtggtggggctggtcgcgtatatcgcgttcaagcgttggaacagctga

SEQ ID NO: 211 - protein, 784 aa, synthetic construct
MALPVTALLLPLALLLHAARPDVVMTQTPLSLPVTLGQPASISCRSSQSLLYSNGNTYLHWYQQRPGQSPRLLIYKLSNRFSGVPDRFSGSGSGTDFTLKISRVEAEDVGVYYCSQSTHVPWTFGGGTKLEIKGGGGSGGGGSGGGGSQITLKESGPTLVKPTQTLTLTCTFSGFSLTTYGIGVGWIRQPPGKALEWLTHIWWNDNKYYSPSLKSRLTITKDSSKNQVVLTMTNMDPVDTATYYCLYGYTYWGQGTLVTVSATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 212 - DNA, 20 bp, synthetic construct
  source 1..20: mol_type = other DNA
cccccatgaacgttaccatt

SEQ ID NO: 213 - DNA, 20 bp, synthetic construct
  source 1..20: mol_type = other DNA
tacttgcgttagtaaacttg

SEQ ID NO: 214 - DNA, 20 bp, synthetic construct
  source 1..20: mol_type = other DNA
tgtcaagtgtaggcctccca

SEQ ID NO: 215 - DNA, 20 bp, synthetic construct
  source 1..20: mol_type = other DNA
gtatttgtggcttaaactct

SEQ ID NO: 216 - DNA, 20 bp, synthetic construct
  source 1..20: mol_type = other DNA
gagtttaagccacaaataca

SEQ ID NO: 217 - DNA, 20 bp, synthetic construct
  source 1..20: mol_type = other DNA
ttaatattagatgtgccacc

SEQ ID NO: 218 - DNA, 20 bp, synthetic construct
  source 1..20: mol_type = other DNA
agcccaacaccttcccccac

SEQ ID NO: 219 - DNA, 20 bp, synthetic construct
  source 1..20: mol_type = other DNA
tggcttaaactcttggcatt

SEQ ID NO: 220 - DNA, 20 bp, synthetic construct
  source 1..20: mol_type = other DNA
gttaatgagaatgtggaatg

SEQ ID NO: 221 - DNA, 20 bp, synthetic construct
  source 1..20: mol_type = other DNA
cttctacaaaaaataatctg

SEQ ID NO: 222 - DNA, 20 bp, synthetic construct
  source 1..20: mol_type = other DNA
tttgtagagaacaacaagca

SEQ ID NO: 223 - DNA, 20 bp, synthetic construct
  source 1..20: mol_type = other DNA
acaaccactctgagcccttc

SEQ ID NO: 224 - DNA, 20 bp, synthetic construct
  source 1..20: mol_type = other DNA
tggaatgtggaaacaatact

SEQ ID NO: 225 - DNA, 20 bp, synthetic construct
  source 1..20: mol_type = other DNA
cataattcatgtactgctcc

SEQ ID NO: 226 - DNA, 20 bp, synthetic construct
  source 1..20: mol_type = other DNA
atatctcataattcatgtac

SEQ ID NO: 227 - DNA, 20 bp, synthetic construct
  source 1..20: mol_type = other DNA
ttaatgagaatgtggaatgt

SEQ ID NO: 228 - DNA, 20 bp, synthetic construct
  source 1..20: mol_type = other DNA
gaaaaatatgcaaacatcac

SEQ ID NO: 229 - DNA, 20 bp, synthetic construct
  source 1..20: mol_type = other DNA
ttgcatatttttcatctaaa

SEQ ID NO: 230 - DNA, 20 bp, synthetic construct
  source 1..20: mol_type = other DNA
tttttcatctaaaagaaaat

SEQ ID NO: 231 - DNA, 20 bp, synthetic construct
  source 1..20: mol_type = other DNA
aaaatatgcaaacatcactg

SEQ ID NO: 232 - DNA, 20 bp, synthetic construct
  source 1..20: mol_type = other DNA
ttaccacatgttggcttaga

SEQ ID NO: 233 - DNA, 20 bp, synthetic construct
  source 1..20: mol_type = other DNA
attaatgtcttatcaggagc

SEQ ID NO: 234 - DNA, 20 bp, synthetic construct
  source 1..20: mol_type = other DNA
gttaatgagaatgtggaatg

SEQ ID NO: 235 - DNA, 20 bp, synthetic construct
  source 1..20: mol_type = other DNA
aaatatgcaaacatcactgt

SEQ ID NO: 236 - DNA, 20 bp, synthetic construct
  source 1..20: mol_type = other DNA
ccacatgttggcttagatgg

SEQ ID NO: 237 - DNA, 20 bp, synthetic construct
  source 1..20: mol_type = other DNA
atctaagccaacatgtggta

SEQ ID NO: 238 - DNA, 20 bp, synthetic construct
  source 1..20: mol_type = other DNA
actccatctaagccaacatg

SEQ ID NO: 239 - DNA, 20 bp, synthetic construct
  source 1..20: mol_type = other DNA
aatgaggtgcataaccttac

SEQ ID NO: 240 - DNA, 20 bp, synthetic construct
  source 1..20: mol_type = other DNA
agtacatgaattatgagata

SEQ ID NO: 241 - DNA, 20 bp, synthetic construct
  source 1..20: mol_type = other DNA
tcattaacatttagctttgc

SEQ ID NO: 242 - DNA, 20 bp, synthetic construct
  source 1..20: mol_type = other DNA
gctaaatgttaatgagaatg

SEQ ID NO: 243 - DNA, 20 bp, synthetic construct
  source 1..20: mol_type = other DNA
cctgataagacattaatatt

SEQ ID NO: 244 - DNA, 20 bp, synthetic construct
  source 1..20: mol_type = other DNA
ttctcattaacatttagctt

SEQ ID NO: 245 - DNA, 20 bp, synthetic construct
  source 1..20: mol_type = other DNA
ccacattctcattaacattt

SEQ ID NO: 246 - DNA, 20 bp, synthetic construct
  source 1..20: mol_type = other DNA
tgcacctcattgtttgtgca

SEQ ID NO: 247 - DNA, 20 bp, synthetic construct
  source 1..20: mol_type = other DNA
caggagcagtacatgaatta

SEQ ID NO: 248 - DNA, 20 bp, synthetic construct
  source 1..20: mol_type = other DNA
taagacattaatattagatg

SEQ ID NO: 249 - DNA, 20 bp, synthetic construct
  source 1..20: mol_type = other DNA
gcacaaacaatgaggtgcat

SEQ ID NO: 250 - DNA, 20 bp, synthetic construct
  source 1..20: mol_type = other DNA
atgagaatgtggaatgtgga

SEQ ID NO: 251 - DNA, 20 bp, synthetic construct
  source 1..20: mol_type = other DNA
aacaatgaggtgcataacct

SEQ ID NO: 252 - DNA, 20 bp, synthetic construct
  source 1..20: mol_type = other DNA
gatgaaaaatatgcaaacat

SEQ ID NO: 253 - DNA, 4818 bp, synthetic construct
  source 1..4818: mol_type = other DNA
atgaaacggacagccgacggaagcgagttcgagtcaccaaagaagaagcggaaagtctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcaggcccaagaggaacagcgataagctgatcgccagaaagaaggactgggaccctaagaagtacggcggcttcgtcagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccagattcctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctagggccttcaagtactttgacaccaccatcgaccggaaggtgtacaggagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtctaa

SEQ ID NO: 254 - protein, 1605 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGDSGGSKRTADGSEFEPKKKRKV

SEQ ID NO: 255 - DNA, 4818 bp, synthetic construct
  source 1..4818: mol_type = other DNA
atgaaacggacagccgacggaagcgagttcgagtcaccaaagaagaagcggaaagtctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagacaagaagtacagcatcggcctgaccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatggtgaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcattatcccccaccagatccacctgggagagctgcacgccattctgcggcggcagggcgatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggctgagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggcggccacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagggtaacagcgataagctgatcgccagaaagaaggactgggaccctaagaagtacggcggcttcaacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgtgctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcaaccggaagcaatacaacacgaccaaagaggtgctggacgccaccctgatccgtcagagcatcaccggcctgtacgagacacggatcgacctgtctcagctgggaggtgactctggcggctcaaaaagaaccgccgacggcagcgaattcgagcccaagaagaagaggaaagtctaa

SEQ ID NO: 256 - protein, 1605 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLTIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMVKRYDEHHQDLTLLKALVRQQLPEKYKEIFFDQSKNGYAGYIDGGASQEEFYKFIKPILEKMDGTEELLVKLNREDLLRKQRTFDNGIIPHQIHLGELHAILRRQGDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRLRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGGHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKGNSDKLIARKKDWDPKKYGGFNSPTVAYSVLVVAKVEKGKSKKLKSVKELLGITIMERSSFEKNPIDFLEAKGYKEVKKDLIIKLPKYSLFELENGRKRMLASAGVLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTINRKQYNTTKEVLDATLIRQSITGLYETRIDLSQLGGDSGGSKRTADGSEFEPKKKRKV

SEQ ID NO: 257 - DNA, 5562 bp, synthetic construct
  source 1..5562: mol_type = other DNA
atgaaacggacagccgacggaagcgagttcgagtcaccaaagaagaagcggaaagtcagttcaaagactgggcctgtcgccgtcgatccaaccctgcgccgccggattgaacctcacgagtttgaagtgttctttgacccccgggagctgagaaaggagacatgcctgctgtacgagatcaactggggaggcaggcactccatctggaggcacacctctcagaacacaaataagcacgtggaggtgaacttcatcgagaagtttaccacagagcggtacttctgccccaataccagatgtagcatcacatggtttctgagctggtccccttgcggagagtgtagcagggccatcaccgagttcctgtccagatatccaaatgtgacactgtttatctacatcgccaggctgtatcacctggcaaacccaaggaataggcagggcctgcgcgatctgatcagctccggcgtgaccatccagatcatgacagagcaggagtccggctactgctggcacaacttcgtgaattattctcctagcaacgagtcccactggcctaggtacccacacctgtgggtgcgcctgtacgtgctggagctgtattgcatcatcctgggcctgcccccttgtctgaatatcctgcggagaaagcagagccagctgacctcctttacaatcgccctgcagtcttgtcactatcagaggctgccaccccacatcctgtgggccacaggcctgaagtctggcggatctagcggaggatcctctggcagcgagacaccaggaacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatccggcccaagaggaacagcgataagctgatcgccagaaagaaggactgggaccctaagaagtacggcggcttcgtgagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgcccggttcctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctcgggccttcaagtactttgacaccaccatcgaccggaaggtgtaccggagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtctaa

SEQ ID NO: 258 - protein, 1853 aa, synthetic construct
MKRTADGSEFESPKKKRKVSSKTGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPNVTLFIYIARLYHLANPRNRQGLRDLISSGVTIQIMTEQESGYCWHNFVNYSPSNESHWPRYPHLWVRLYVLELYCIILGLPPCLNILRRKQSQLTSFTIALQSCHYQRLPPHILWATGLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 259 - DNA, 27 bp, synthetic construct
  source 1..27: mol_type = other DNA
gaaaaatatgcaaacatcactgtggat

SEQ ID NO: 260 - DNA, 21 bp, synthetic construct
  source 1..21: mol_type = other DNA
ccatctaagccaacatgtggt

SEQ ID NO: 261 - DNA, 24 bp, synthetic construct
  source 1..24: mol_type = other DNA
tccatatctcataattcatgtact

SEQ ID NO: 262 - protein, 9 aa, synthetic construct
EKYADITVD

SEQ ID NO: 263 - protein, 6 aa, synthetic construct
PSKPTC

SEQ ID NO: 264 - protein, 8 aa, synthetic construct
SISHNSCT

SEQ ID NO: 265 - DNA, 12 bp, synthetic construct
  source 1..12: mol_type = other DNA
aacccacgcacc

SEQ ID NO: 266 - protein, 4 aa, synthetic construct
NPRT

SEQ ID NO: 267 - DNA, 20 bp, synthetic construct
  source 1..20: mol_type = other DNA
aaaatatgcaaacatcactg

SEQ ID NO: 268 - DNA, 21 bp, synthetic construct
  source 1..21: mol_type = other DNA
gataaatctggtcttatttcc

SEQ ID NO: 269 - protein, 76 aa, synthetic construct
XEKYAXIXVDYLYNXXTKLFTAKLNVNENVECXNNXXXXXXCTNNEVXNLXECKXXXVXXSHNSCTXXXKXLXLDV

SEQ ID NO: 270 - protein, 75 aa, synthetic construct
AEKYATIPVDYLYNNKTKLFTAKLNVNENVECTNNNHTHNICTNNEVLNLPECKEMNVFVSHNSCTDRHKELKLD

SEQ ID NO: 271 - protein, 71 aa, synthetic construct
DEKYANITVDYLYNKETKLFTAKLNVNENVECGNNTCTNNEVHNLTECKNASVSISHNSCTAPDKTLILDV

SEQ ID NO: 272 - DNA, 23 bp, synthetic construct
  source 1..23: mol_type = other DNA
aaaatatgcaaacatcactgtgg

SEQ ID NO: 273 - DNA, 23 bp, synthetic construct
  source 1..23: mol_type = other DNA
aaaatatgcaaccatccctgtgg

SEQ ID NO: 274 - protein, 7 aa, synthetic construct
KYANITV

SEQ ID NO: 275 - protein, 7 aa, synthetic construct
KYATIPV

SEQ ID NO: 276 - DNA, 23 bp, synthetic construct
  source 1..23: mol_type = other DNA
ccatatctcataattcatgtact

SEQ ID NO: 277 - DNA, 23 bp, synthetic construct
  source 1..23: mol_type = other DNA
tcgtatctcataattcatgtaca

SEQ ID NO: 278 - protein, 7 aa, synthetic construct
ISHNSCT

SEQ ID NO: 279 - protein, 7 aa, synthetic construct
VSHNSCT

SEQ ID NO: 280 - DNA, 24 bp, synthetic construct
  source 1..24: mol_type = other DNA
agaagaagcctttgggaccaagga

SEQ ID NO: 281 - protein, 8 aa, synthetic construct
RRSLWDQG

SEQ ID NO: 282 - protein, 31 aa, synthetic construct
DYQVSSPIYDINYYTSEPCQKINVKQIAARL

SEQ ID NO: 283 - protein, 62 aa, synthetic construct
TCAAMFGNITVNYTYESSNQTFKADLKDVQNAKCGNEDCENVLNNLEECSQIKNISVSNDSC

SEQ ID NO: 284 - protein, 63 aa, synthetic construct
TCDEKYANITVDYLYNKETKLFTAKLNVNENVECGNNTCTNNEVHNLTECKNASVSISHNSCT

SEQ ID NO: 285 - protein, 8 aa, synthetic construct
TEKKNDEW

SEQ ID NO: 286 - protein, 8 aa, synthetic construct
TEKKNDEA

SEQ ID NO: 287 - protein, 8 aa, synthetic construct
TEKKNDAW

SEQ ID NO: 288 - protein, 8 aa, synthetic construct
TEKKNAEW

SEQ ID NO: 289 - protein, 8 aa, synthetic construct
TEKKADEW

SEQ ID NO: 290 - protein, 8 aa, synthetic construct
TEKANDEW

SEQ ID NO: 291 - protein, 8 aa, synthetic construct
TEAKNDEW

SEQ ID NO: 292 - protein, 8 aa, synthetic construct
TAKKNDEW

SEQ ID NO: 293 - protein, 8 aa, synthetic construct
AEKKNDEW

SEQ ID NO: 294 - DNA, 30 bp, synthetic construct
  source 1..30: mol_type = other DNA
ccttcccccactggattgactacagcaaag

SEQ ID NO: 295 - protein, 10 aa, synthetic construct
PSPTGLTTAK

SEQ ID NO: 296 - DNA, 20 bp, synthetic construct
  source 1..20: mol_type = other DNA
taatccatcttgttccaccc

SEQ ID NO: 297 - DNA, 23 bp, synthetic construct
  source 1..23: mol_type = other DNA
tcagtttacacccgatccactgg

SEQ ID NO: 298 - DNA, 23 bp, synthetic construct
  source 1..23: mol_type = other DNA
agtttacacccgatccactgggg

SEQ ID NO: 299 - DNA, 23 bp, synthetic construct
  source 1..23: mol_type = other DNA
tcatcctcctgacaatcgatagg

SEQ ID NO: 300 - DNA, 23 bp, synthetic construct
  source 1..23: mol_type = other DNA
caatgtgtcaactcttgacaggg

SEQ ID NO: 301 - DNA, 20 bp, synthetic construct
  source 1..20: mol_type = other DNA
taataattgatgtcatagat

SEQ ID NO: 302 - DNA, 20 bp, synthetic construct
  source 1..20: mol_type = other DNA
tgacatcaattattatacat

SEQ ID NO: 303 - DNA, 20 bp, synthetic construct
  source 1..20: mol_type = other DNA
ctccgatgtataataattga

SEQ ID NO: 304 - DNA, 20 bp, synthetic construct
  source 1..20: mol_type = other DNA
tgtataataattgatgtcat

SEQ ID NO: 305 - DNA, 20 bp, synthetic construct
  source 1..20: mol_type = other DNA
ttatcaggatgaggatgacc

SEQ ID NO: 306 - DNA, 20 bp, synthetic construct
  source 1..20: mol_type = other DNA
cattaaagatagtcatcttg

SEQ ID NO: 307 - DNA, 20 bp, synthetic construct
  source 1..20: mol_type = other DNA
aagaaggtcttcattacacc

SEQ ID NO: 308 - DNA, 20 bp, synthetic construct
  source 1..20: mol_type = other DNA
acagcatttgcagaagcgtt

SEQ ID NO: 309 - DNA, 20 bp, synthetic construct
  source 1..20: mol_type = other DNA
gggacacagggttaatgtga

SEQ ID NO: 310 - DNA, 20 bp, synthetic construct
  source 1..20: mol_type = other DNA
aatcctgccacctatgtatc

SEQ ID NO: 311 - DNA, 20 bp, synthetic construct
  source 1..20: mol_type = other DNA
tgcaggtgacagagactctt

SEQ ID NO: 312 - DNA, 20 bp, synthetic construct
  source 1..20: mol_type = other DNA
ccaaacgcttctgcaaatgc

SEQ ID NO: 313 - DNA, 20 bp, synthetic construct
  source 1..20: mol_type = other DNA
aagttcagaaactacctctt

SEQ ID NO: 314 - DNA, 20 bp, synthetic construct
  source 1..20: mol_type = other DNA
agacatgccgctgctgctac

SEQ ID NO: 315 - DNA, 22 bp, synthetic construct
  source 1..22: mol_type = other DNA
agacatgccgctgctgctactg

SEQ ID NO: 316 - protein, 7 aa, synthetic construct
DMPLLLL

SEQ ID NO: 317 - DNA, 20 bp, synthetic construct
  source 1..20: mol_type = other DNA
acttacaggtgacgttgagc

SEQ ID NO: 318 - DNA, 27 bp, synthetic construct
  source 1..27: mol_type = other DNA
ccagctcaacgtcacctgtaagtgctg

SEQ ID NO: 319 - protein, 5 aa, synthetic construct
QLNVT

SEQ ID NO: 320 - DNA, 20 bp, synthetic construct
  source 1..20: mol_type = other DNA
ttcctacctgagccatctcc

SEQ ID NO: 321 - DNA, 26 bp, synthetic construct
  source 1..26: mol_type = other DNA
ccaggagatggctcaggtaggaagga

SEQ ID NO: 322 - protein, 5 aa, synthetic construct
PGDGS

SEQ ID NO: 323 - DNA, 20 bp, synthetic construct
  source 1..20: mol_type = other DNA
cactcacctgcccacagcag

SEQ ID NO: 324 - DNA, 27 bp, synthetic construct
  source 1..27: mol_type = other DNA
cccctgctgtgggcaggtgagtggctg

SEQ ID NO: 325 - protein, 5 aa, synthetic construct
PLLWA

(19) United States
(12) Patent Application Publication
(10) Pub. No.: US 2025/0041339 A1
(43) Pub. Date: Feb. 6, 2025

(54) Engineered Pan-Leukocyte Antigen CD45 to Facilityate CAR T Cell Therapy

(71) Applicant: The Trustees of the University of Pennsylvania, Philadelphia, PA (US)

(72) Inventors: Saar Gill, Philadelphia, PA (US); Carl H. June, Merion Station, PA (US); Nils Wellhausen, Philadelphia, PA (US)

(21) Appl. No.: 18/711,503

(22) PCT Filed: Nov. 16, 2022
(86) PCT No.: PCT/US2022/079970

§ 371 Date: May 17, 2024

Related U.S. Application Data
(60) Provisional application No. 63/281,547, filed on Nov. 19, 2021.

(51) Int. Cl. A61K 35/17; A61K 39/00; A61P 35/00; C07K 14/705; C07K 14/725; C07K 16/28; C12N 5/0783

(52) U.S. Cl. CPC A61K 35/17; A61K 39/4611; A61K 39/4631; A61K 39/464429; A61P 35/00; C07K 14/7051; C07K 14/70517; C07K 16/289; C12N 5/0636; A61K 2239/13; A61K 2239/21; C07K 2317/565; C07K 2317/622; C07K 2319/03

(57) ABSTRACT

The present disclosure provides modified immune cells or precursors thereof (e.g., gene edited modified T cells) comprising chimeric antigen receptors (CARs) specific for CD45. In certain embodiments, the modified immune cells or precursors thereof further comprise or instead comprise a modified endogenous gene locus encoding CD45.

### CROSS-REFERENCE TO RELATED APPLICATION

The present application is a 35 U.S.C. § 371 national phase application from, and claims priority to, International Application No. PCT/US2022/079970 filed Nov. 16, 2022, and is entitled to priority under 35 U.S.C. § 119(e) to U.S. Provisional Patent Application No. 63/281,547, filed Nov. 19, 2021, which is hereby incorporated by reference in its entirety herein.

### STATEMENT REGARDING FEDERALLY SPONSORED RESEARCH OR DEVELOPMENT

This invention was made with government support under CA214278 awarded by the National Institutes of Health. The government has certain rights in the invention.

### REFERENCE TO AN ELECTRONIC SEQUENCE LISTING

The Sequence Listing submitted herewith as a xml file named “046483-7344US1-Sequence-Listing,” created on May 10, 2024 and having a size of 452,383 bytes, is incorporated herein by reference in its entirety.

### BACKGROUND OF THE INVENTION

The development of the chimeric antigen receptor (CAR) and its successful clinical use to direct T cells against specific types of cancers has been an important advancement in cancer immunotherapy. Once confined to experimental laboratory proofs of concept, immunotherapies including CAR T cells are now considered first-line therapies. Despite the efficacy of this approach, CAR constructs must be individually designed and developed for each disease, and target molecules must be selected that minimize unintended toxicities to normal cell populations which also express them, a requirement which decreases efficiency and increases the costs associated with CAR therapy. By targeting a pan-hematologic antigen, a single CAR T cell population could be used for all hematologic malignancies, thereby accelerating clinical research and development and expediting the treatment of more patients. CD45 is a receptor tyrosine phosphatase that is expressed on the surface of most hematopoietic cells, from the most immature stem cell to the most differentiated progeny, and antibody-drug conjugates targeting CD45 cause profound myeloablation. A unique hurtle to developing CAR T cells directed against CD45 is that T cell themselves express CD45. While CD45 could be genetically deleted in order to circumvent this challenge, the known role of CD45 in the immune synapse suggests that this modification would result in major disturbances to immune function. Thus, there is a need in the art for CAR T cell-based treatment strategies that combine the cytotoxic efficacy of anti-CD45 CAR T cells with gene-editing that results in hematopoietic cells which lack the CAR-targeted CD45 epitope. The present invention addresses this need.

### SUMMARY OF THE INVENTION

As described herein, the present invention relates to modified immune cells or precursors thereof (e.g., gene edited modified T cells) comprising chimeric antigen receptors (CARs) specific for CD45. In certain embodiments, the modified immune cells or precursors thereof further comprise or instead comprise a modified endogenous gene locus encoding CD45.

As such, in one aspect, the invention provides a chimeric antigen receptor (CAR) comprising an antigen binding domain, a transmembrane domain, and an intracellular domain, wherein the antigen binding domain comprises:
i. a heavy chain variable region that comprises three heavy chain complementarity determining regions (HCDRs), wherein HCDR1 comprises an amino acid sequence selected from the group consisting of SEQ ID NOs: 89, 92, 95, 98, 101, 104, 107, 110, 113, and 116, HCDDR2 comprises an amino acid sequence selected from the group consisting of SEQ ID NOs: 90, 93, 96, 99, 102, 105, 108, 111, 114, and 117, and HCDR3 comprises and amino acid sequence selected from the group comprising SEQ ID NOs: 91, 94, 97, 100, 103, 106, 109, 112, 115, and 118; and
ii. a light chain variable region that comprises three light chain complementarity determining regions (LCDRs), wherein LCDR1 comprises an amino acid sequence selected from the group consisting of SEQ ID NOs: 119, 122, 125, 128, 131, 134, 137, 140, and 143, LCDR2 comprises and amino acid sequence selected from the group consisting of SEQ ID NOs: 120, 123, 126, 129, 132, 135, 138, 141, and 144, and LCDR3 comprises an amino acid sequence selected from the group consisting of SEQ ID NOs: 121, 124, 127, 130, 133, 136, 139, 142, and 145.

In certain embodiments, the CAR binds CD45.

In certain embodiments, the antigen binding domain comprises an antibody or an antigen-binding fragment thereof.

In certain embodiments, the antigen-binding fragment is selected from the group consisting of a Fab, a single-chain variable fragment (scFv), or a single-domain antibody.

In certain embodiments, the antigen-binding fragment is a scFv.

In certain embodiments, the antigen binding domain comprises a heavy chain variable region comprising an amino acid sequence having at least 95%-99% identity to the amino acid sequence of any of the heavy chain variable regions set forth in SEQ ID NOs: 146-155.

In certain embodiments, the antigen binding domain comprises a heavy chain variable region comprising an amino acid sequence of any of the heavy chain variable regions set forth in SEQ ID NOs: 146-155.

In certain embodiments, the antigen binding domain comprises a heavy chain variable region consisting of an amino acid sequence of any of the heavy chain variable regions set forth in SEQ ID NOs: 146-155.

In certain embodiments, the antigen binding domain comprises a light chain variable region comprising an amino acid sequence having at least 95-99% identity to the amino acid sequence of any of the light chain variable regions set forth in SEQ ID NOs: 156-165.

In certain embodiments, the antigen binding domain comprises a light chain variable region comprising an amino acid sequence of any of the light chain variable regions set forth in SEQ ID NOs: 156-165.

In certain embodiments, the antigen binding domain comprises a light chain variable region consisting of an amino acid sequence of any of the light chain variable regions set forth in SEQ ID NOs: 156-165.

In another aspect, the invention includes a chimeric antigen receptor (CAR) comprising an antigen binding domain, a transmembrane domain, and an intracellular domain, wherein the antigen binding domain comprises a heavy chain variable region comprising any of the amino acid sequences set forth in SEQ ID NO: 146-155; and a light chain variable region comprising any of the amino acid sequences set forth in SEQ ID NO: 156-165.

In another aspect, the invention includes a chimeric antigen receptor (CAR) comprising an antigen binding domain, a transmembrane domain, and an intracellular signaling domain, wherein the antigen binding domain comprises any one of the amino acid sequences set forth in SEQ ID NOs: 175, 177, 179, 181, 183, 185, 187, 189, 191, 193, 195, 197, 199, 201, 203, 205, 207, 209, and 211.

In certain embodiments, the transmembrane domain is selected from the group consisting of an artificial hydrophobic sequence, and a transmembrane domain of a type I transmembrane protein, an alpha, beta, or zeta chain of a T cell receptor, CD28, CD3 epsilon, CD45, CD4, CD5, CD8, CD9, CD16, CD22, CD33, CD37, CD64, CD80, CD86, OX40 (CD134), 4-1BB (CD137), and CD154.

In certain embodiments, the transmembrane domain comprises a transmembrane domain of CD8 comprising an amino acid sequence set forth in SEQ ID NO: 169.

In certain embodiments of the above aspects or any aspect or embodiment disclosed herein, the CAR further comprises a hinge domain.

In certain embodiments, the hinge domain is selected from the group consisting of an Fc fragment of an antibody, a hinge region of an antibody, a CH2 region of an antibody, a CH3 region of an antibody, an artificial hinge domain, a hinge comprising an amino acid sequence of a CD8 hinge, or any combination thereof.

In certain embodiments, the hinge domain is a CD8 hinge comprising an amino acid sequence set forth in SEQ ID NO: 168.

In certain embodiments, the intracellular domain comprises a costimulatory signaling domain and an intracellular signaling domain.

In certain embodiments, the costimulatory signaling domain comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFI-II, Fas, CD30, CD40, ICOS, NKG2C, and B7-H3 (CD276), or a variant thereof.

In certain embodiments, the costimulatory signaling domain comprises a costimulatory domain of 4-1BB comprising an amino acid sequence set forth in SEQ ID NO: 170.

In certain embodiments, the intracellular signaling domain comprises an intracellular domain selected from the group consisting of cytoplasmic signaling domains of a human CD3 zeta chain (CD3ζ), FeyRIII, FesRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

In certain embodiments, the intracellular signaling domain comprises an intracellular domain of CD3ζ.

In certain embodiments, the CD3ζ intracellular domain comprises an amino acid sequence set forth in SEQ ID NO: 171.

In another aspect, the invention includes a chimeric antigen receptor (CAR) comprising:
i. an antigen binding domain comprising: a heavy chain variable region that comprises three heavy chain complementarity determining regions (HCDRs), wherein HCDR1 comprises an amino acid sequence selected from the group consisting of SEQ ID NOs: 89, 92, 95, 98, 101, 104, 107, 110, 113, and 116, HCDDR2 comprises an amino acid sequence selected from the group consisting of SEQ ID NOs: 90, 93, 96, 99, 102, 105, 108, 111, 114, and 117, and HCDR3 comprises and amino acid sequence selected from the group comprising SEQ ID NOs: 91, 94, 97, 100, 103, 106, 109, 112, 115, and 118; and a light chain variable region that comprises three light chain complementarity determining regions (LCDRs), wherein LCDR1 comprises an amino acid sequence selected from the group consisting of SEQ ID NOs: 119, 122, 125, 128, 131, 134, 137, 140, and 143, LCDR2 comprises and amino acid sequence selected from the group consisting of SEQ ID NOs: 120, 123, 126, 129, 132, 135, 138, 141, and 144, and LCDR3 comprises an amino acid sequence selected from the group consisting of SEQ ID NOs: 121, 124, 127, 130, 133, 136, 139, 142, and 145;
ii. a CD8 transmembrane domain;
iii. a 4-1BB costimulatory domain; and
iv. a CD3ζ intracellular signaling domain.

In another aspect, the invention includes a chimeric antigen receptor (CAR) comprising:
i. an antigen binding domain comprising: a heavy chain variable region comprising any of the amino acid sequences set forth in SEQ ID NOs: 146-155; and a light chain variable region comprising any of the amino acid sequences set forth in SEQ ID NOs: 156-165;
ii. a CD8 transmembrane domain;
iii. a 4-1BB costimulatory domain; and
iv. a CD3ζ intracellular signaling domain.

In another aspect, the invention includes a chimeric antigen receptor (CAR) comprising any one of the amino acid sequences set forth in SEQ ID NOs: 175, 177, 179, 181, 183, 185, 187, 189, 191, 193, 195, 197, 199, 201, 203, 205, 207, 209, and 211.

In another aspect, the invention includes a chimeric antigen receptor (CAR) consisting of any one of the amino acid sequences set forth in SEQ ID NOs: 175, 177, 179, 181, 183, 185, 187, 189, 191, 193, 195, 197, 199, 201, 203, 205, 207, 209, and 211.

In another aspect, the invention includes a nucleic acid encoding the CAR of any one of the above aspects or any aspect or embodiment disclosed herein.

In another aspect, the invention includes a nucleic acid encoding a chimeric antigen receptor (CAR) comprising an antigen binding domain, a transmembrane domain, and an intracellular domain, wherein the antigen binding domain comprises:
i. a heavy chain variable region that comprises three heavy chain complementarity determining regions (HCDRs), wherein HCDR1 comprises an amino acid sequence selected from the group consisting of SEQ ID NOs: 89, 92, 95, 98, 101, 104, 107, 110, 113, and 116, HCDDR2 comprises an amino acid sequence selected from the group consisting of SEQ ID NOs: 90, 93, 96, 99, 102, 105, 108, 111, 114, and 117, and HCDR3 comprises and amino acid sequence selected from the group comprising SEQ ID NOs: 91, 94, 97, 100, 103, 106, 109, 112, 115, and 118; and
ii. a light chain variable region that comprises three light chain complementarity determining regions (LCDRs), wherein LCDR1 comprises an amino acid sequence selected from the group consisting of SEQ ID NOs: 119, 122, 125, 128, 131, 134, 137, 140, and 143, LCDR2 comprises and amino acid sequence selected from the group consisting of SEQ ID NOs: 120, 123, 126, 129, 132, 135, 138, 141, and 144, and LCDR3 comprises an amino acid sequence selected from the group consisting of SEQ ID NOs: 121, 124, 127, 130, 133, 136, 139, 142, and 145.

In certain embodiments, the CAR binds CD45.

In certain embodiments, the antigen binding domain comprises an antibody or an antigen-binding fragment thereof.

In certain embodiments, the antigen-binding fragment is selected from the group consisting of a Fab, a single-chain variable fragment (scFv), or a single-domain antibody.

In certain embodiments, the antibody is a scFv.

In another aspect, the invention includes a nucleic acid encoding a chimeric antigen receptor (CAR) comprising an antigen binding domain, a transmembrane domain, and an intracellular signaling domain, wherein the antigen binding domain comprises:
i. a heavy chain variable region comprising a nucleic acid encoding any of the amino acid sequences set forth in SEQ ID NOs: 146-155; and
ii. a light chain variable region comprising a nucleic acid encoding any of the amino acid sequences set forth in SEQ ID NOs: 156-165.

In another aspect, the invention includes a nucleic acid encoding a chimeric antigen receptor (CAR) comprising an antigen binding domain, a transmembrane domain, and an intracellular signaling domain, wherein the antigen binding domain is encoded by any one of the nucleotide sequences set forth in SEQ ID NOs: 174, 176, 178, 180, 182, 184, 186, 188, 190, 192, 194, 196, 198, 200, 202, 204, 208, and 210.

In certain embodiments, the transmembrane domain is selected from the group consisting of an artificial hydrophobic sequence, and a transmembrane domain of a type I transmembrane protein, an alpha, beta, or zeta chain of a T cell receptor, CD28, CD3epsilon, CD45, CD4, CD5, CD8, CD9, CD16, CD22, CD33, CD37, CD64, CD80, CD86, OX40 (CD134), 4-1BB (CD137), and CD154.

In certain embodiments, the transmembrane domain comprises a transmembrane domain of CD8.

In certain embodiments, the CAR further comprises a hinge domain.

In certain embodiments, the hinge domain is selected from the group consisting of an Fc fragment of an antibody, a hinge region of an antibody, a CH2 region of an antibody, a CH3 region of an antibody, an artificial hinge domain, a hinge comprising an amino acid sequence of a CD8 hinge, or any combination thereof.

In certain embodiments, the artificial hinge domain is a CD8 hinge.

In certain embodiments, the intracellular domain comprises a costimulatory signaling domain and an intracellular signaling domain.

In certain embodiments, the costimulatory signaling domain comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFI-II, Fas, CD30, CD40, ICOS, NKG2C, and B7-H3 (CD276), or a variant thereof.

In certain embodiments, the costimulatory signaling domain comprises a costimulatory domain of 4-1BB.

In certain embodiments, the intracellular signaling domain comprises an intracellular domain selected from the group consisting of cytoplasmic signaling domains of a human CD3 zeta chain (CD3ζ), FeyRIII, FesRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

In certain embodiments, the intracellular signaling domain comprises an intracellular domain of CD3ζ, In another aspect, the invention includes a vector comprising the nucleic acid of any one of the above aspects or any aspect or embodiment disclosed herein.

In certain embodiments, the vector is an expression vector.

In certain embodiments, the vector further comprising a CRISPR-based gene editing system.

In certain embodiments, the CRISPR-based gene editing system downregulates the expression of endogenous CD45.

In certain embodiments, the CRISPR-based gene editing system alters the epitope of CD45 recognized by the CAR.

In certain embodiments, the alteration of the epitope of CD45 renders it unable to be bound by the CAR.

In certain embodiments, the alteration is selected from the group consisting of a substitution, an insertion, a deletion, and an insertion/deletion.

In certain embodiments, the alteration is mediated by a CRISPR system comprising a CRISPR nuclease and a guide RNA.

In certain embodiments, the CRISPR nuclease is CRISPR/Cas9.

In certain embodiments, the guide RNA comprises a guide sequence that is sufficiently complementary with a target sequence in the endogenous gene locus encoding CD45.

In certain embodiments, the guide RNA comprises a nucleic acid sequence set forth in any one of SEQ ID NOs: 212-223.

In certain embodiments, the modification is mediated by a CRISPR system comprising a base editor system.

In certain embodiments, the base editor system comprises a single guide RNA.

In certain embodiments, the single guide RNA comprises a nucleic acid sequence set forth in any one of SEQ ID NOs: 224-252.

In certain embodiments, the base editor system comprises a nucleic acid sequence set forth in any one of SEQ ID NOs: 253-258.

In another aspect, the invention includes a cell comprising the CAR of any one of the above aspects or embodiments, the nucleic acid of any one of the above aspects or embodiments, or the vector of any one of the above aspects or embodiments.

In certain embodiments, the cell is selected from the group consisting of a T cell, a natural killer (NK) cell, a cytotoxic T lymphocyte (CTL), and a regulatory T cell.

In another aspect, the invention includes a modified immune cell or precursor cell thereof, comprising a chimeric antigen receptor (CAR) comprising an antigen binding domain, a transmembrane domain, and an intracellular domain, wherein the antigen binding domain comprises:
i. a heavy chain variable region that comprises three heavy chain complementarity determining regions (HCDRs), wherein HCDR1 comprises an amino acid sequence selected from the group consisting of SEQ ID NOs: 89, 92, 95, 98, 101, 104, 107, 110, 113, and 116, HCDDR2 comprises an amino acid sequence selected from the group consisting of SEQ ID NOs: 90, 93, 96, 99, 102, 105, 108, 111, 114, and 117, and HCDR3 comprises and amino acid sequence selected from the group comprising SEQ ID NOs: 91, 94, 97, 100, 103, 106, 109, 112, 115, and 118;
ii. a light chain variable region that comprises three light chain complementarity determining regions (LCDRs), wherein LCDR1 comprises an amino acid sequence selected from the group consisting of SEQ ID NOs: 119, 122, 125, 128, 131, 134, 137, 140, and 143, LCDR2 comprises and amino acid sequence selected from the group consisting of SEQ ID NOs: 120, 123, 126, 129, 132, 135, 138, 141, and 144, and LCDR3 comprises an amino acid sequence selected from the group consisting of SEQ ID NOs: 121, 124, 127, 130, 133, 136, 139, 142, and 145.
iii. a CRISPR-mediated modification in an endogenous gene locus encoding CD45.

In certain embodiments, the invention includes the modified immune cell of any of the above aspects or embodiments, wherein the CAR binds CD45.

In certain embodiments, the CRISPR-mediated modification is a deletion that downregulates the expression of endogenous CD45.

In certain embodiments, the CRISPR-mediated modification alters the epitope of CD45 recognized by the CAR.

In certain embodiments, the alteration of the epitope of CD45 renders it unable to be bound by the CAR.

In certain embodiments, the modification is selected from the group consisting of a substitution, an insertion, a deletion, and an insertion/deletion.

In certain embodiments, the modification is mediated by a CRISPR system comprising a CRISPR nuclease and a guide RNA.

In certain embodiments, the CRISPR nuclease is CRISPR/Cas9.

In certain embodiments, the guide RNA comprises a guide sequence that is sufficiently complementary with a target sequence in the endogenous gene locus encoding CD45.

In certain embodiments, the guide RNA comprises a nucleic acid sequence set forth in any one of SEQ ID NOs: 212-223.

In certain embodiments, the modification is mediated by a CRISPR system comprising a base editor system.

In certain embodiments, the base editor system comprises a single guide RNA.

In certain embodiments, the single guide RNA comprises a nucleic acid sequence set forth in any one of SEQ ID NOs: 224-252.

In certain embodiments, the base editor system comprises a nucleic acid sequence set forth in any one of SEQ ID NOs: 253-258.

In certain embodiments, the modified endogenous gene locus of CD45 comprises a nucleotide sequence set forth in any one of SEQ ID NOs: 3, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, and 87.

In certain embodiments, the modified endogenous gene locus of CD45 encodes a polypeptide comprising the amino acid sequence set forth in any one of SEQ ID NOs: 4, 8, 10, 12, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, and 88.

In certain embodiments, the CAR comprises an antigen binding domain selected from the group consisting of an antibody, an scFv, and a Fab.

In certain embodiments, the CAR further comprises a hinge domain.

In certain embodiments, the hinge domain selected from the group consisting of an Fc fragment of an antibody, a hinge region of an antibody, a CH2 region of an antibody, a CH3 region of an antibody, an artificial hinge domain, a hinge comprising an amino acid sequence of CD8, or any combination thereof.

In certain embodiments, the CAR comprises a transmembrane domain selected from the group consisting of an artificial hydrophobic sequence and transmembrane domain of a type I transmembrane protein, an alpha, beta, or zeta chain of a T cell receptor, CD28, CD3 epsilon, CD45, CD4, CD5, CD8, CD9, CD16, CD22, CD33, CD37, CD64, CD80, CD86, CD134, CD137, and CD154.

In certain embodiments, the CAR comprises at least one co-stimulatory domain selected from the group consisting of co-stimulatory domains of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3.

In certain embodiments, the CAR comprises an intracellular domain comprising an intracellular domain selected from the group consisting of cytoplasmic signaling domains of a human CD3 zeta chain, FcγRIII, FcsRI, a cytoplasmic tail of an Fc receptor, an immunoreceptor tyrosine-based activation motif (ITAM) bearing cytoplasmic receptors, TCR zeta, FcR gamma, FcR beta, CD3 gamma, CD3 delta, CD3 epsilon, CD5, CD22, CD79a, CD79b, and CD66d.

In certain embodiments, the modified cell is resistant to CAR T cell fratricide.

In certain embodiments, the modified cell is an autologous cell.

In certain embodiments, the modified cell is a cell isolated from a human subject.

In certain embodiments, the modified cell is a modified T cell.

In another aspect, the invention includes a method for generating a modified immune cell or precursor cell thereof, comprising:
introducing into an immune or precursor cell a CRISPR system comprising one or more polypeptides and/or nucleic acids capable of modifying expression of endogenous CD45 gene; and
introducing into the immune or precursor cell a nucleic acid encoding a chimeric antigen receptor (CAR), wherein the CAR has affinity for an antigen on a target cell.

In certain embodiments, the one or more polypeptides and/or nucleic acids capable of downregulating gene expression of endogenous CD45 introduces a CRISPR-mediated modification in an endogenous gene locus encoding CD45.

In certain embodiments, the modification is selected from the group consisting of a substitution, an insertion, a deletion, and an insertion/deletion.

In certain embodiments, the CRISPR system comprises a CRISPR nuclease and a guide RNA.

In certain embodiments, the CRISPR nuclease is Cas9.

In certain embodiments, the guide RNA comprises a guide sequence that is sufficiently complementary with a target sequence in the endogenous gene locus encoding CD45.

In certain embodiments, the guide RNA comprises a nucleic acid sequence set forth in any one of SEQ ID NO: 212-223.

In certain embodiments, the CRISPR system comprises a base editor system.

In certain embodiments, the base editor system comprises a single guide RNA.

In certain embodiments, the single guide RNA comprises a nucleic acid sequence set forth in any one of SEQ ID NOs: 224-252.

In certain embodiments, the base editor system comprises a nucleic acid sequence set forth in any one of SEQ ID NOs: 253-258.

In another aspect, the invention includes a method of treating cancer in a subject in need thereof, comprising:
i. administering to the subject a modified immune cell of claims 51-76, or a modified immune or precursor cell generated by the method of claims 77-87; and
ii. administering to the subject a modified precursor cell comprising a CRISPR-mediated modification in an endogenous gene locus encoding CD45.

In certain embodiments, the modified precursor cell is selected from the group consisting of a bone marrow stem cell, a hematopoietic progenitor cell, and a cord blood stem cell.

In certain embodiments, the subject is conditioned by pre-treatment with radiation.

In certain embodiments, the subject conditioned by pre-treatment with a chemotherapy.

In certain embodiments, the subject is conditioned by pre-treatment with radiation and chemotherapy.

In certain embodiments, the chemotherapy is selected from the group consisting of anti-thymocyte globulin, carmustine, bulsulfan, carboplatin, cyclosporin A, clofarabine, cyclophosphamide, etoposide, fludarabine, melphalan, methotrexate, tacrolimus, thiotepa, topotecan, or any combination thereof.

In certain embodiments, the cancer is a hematologic cancer.

In certain embodiments, the hematologic cancer is selected from the group consisting of a B-cell lymphoma, a B-cell leukemia, a multiple myeloma, and an acute myeloid leukemia.

In certain embodiments, the modified precursor cell is administered first.

In certain embodiments, the modified precursor cell and modified immune cell are administered concurrently.

In another aspect, the invention includes a method of conditioning a subject prior to bone marrow transplant, comprising administering to the subject an effective amount of a modified T cell comprising a modified immune cell of any of the aspects or embodiments disclosed herein, or a modified immune or precursor cell generated by the method of any of the aspects or embodiments disclosed herein.

In certain embodiments, conditioning further comprises administering an effective amount of a chemotherapy, radiation, or a combination thereof.

In certain embodiments, the chemotherapy, radiation, or combination thereof is administered prior to administration of the modified T cell or modified immune cell.

In certain embodiments, the chemotherapy is selected from the group consisting of anti-thymocyte globulin, carmustine, bulsulfan, carboplatin, cyclosporin A, clofarabine, cyclophosphamide, etoposide, fludarabine, melphalan, methotrexate, tacrolimus, thiotepa, topotecan, or any combination thereof.

In another aspect, the invention includes a method of treating an HIV infection in a subject, comprising:
i. administering to the subject a modified immune cell of above aspects or any aspect or embodiment disclosed herein, or a modified immune or precursor cell generated by the method of any of the above aspects or any aspect or embodiment disclosed herein; and
ii. administering to the subject a modified precursor cell comprising a CRISPR-mediated modification in a first endogenous gene locus and one or more second endogenous gene loci, thereby treating the HIV infection.

In certain embodiments, the first endogenous gene locus is CD45.

In certain embodiments, the one or more second endogenous gene loci are selected from the group consisting of CD4, CCR5, CXCR4, and any combination thereof.

In certain embodiments, the CRISPR-mediated modification is selected from the group consisting of a substitution, an insertion, a deletion, and an insertion/deletion.

In certain embodiments, the CRISPR-mediated modification of the one or more second endogenous gene loci reduces or eliminates the binding of the protein encoded by the loci with HIV gp120.

In certain embodiments, the modified precursor cell is selected from the group consisting of a bone marrow stem cell, a hematopoietic progenitor cell, and a cord blood stem cell.

In certain embodiments, the subject is conditioned by pre-treatment with radiation.

In certain embodiments, the subject conditioned by pre-treatment with a chemotherapy.

In certain embodiments, the subject is conditioned by pre-treatment with radiation and chemotherapy.

In certain embodiments, the chemotherapy is selected from the group consisting of anti-thymocyte globulin, carmustine, bulsulfan, carboplatin, cyclosporin A, clofarabine, cyclophosphamide, etoposide, fludarabine, melphalan, methotrexate, tacrolimus, thiotepa, topotecan, or any combination thereof.

In another aspect, the invention includes a method of treating a hematologic malignancy in a subject in need thereof, comprising:
i. administering to the subject a CD33-targeted therapy comprising a CD33-specific CAR-T cell; and
ii. administering to the subject a population of modified precursor cells comprising a modification in an endogenous gene locus such that the precursor cells are resistant to the CD33-targeted therapy.

In certain embodiments, the endogenous gene locus is CD33.

In certain embodiments, the modification is a CRISPR-mediated modification.

In certain embodiments, the CRISPR-mediated modification is selected from the group consisting of a substitution, an insertion, a deletion, and an insertion/deletion.

In certain embodiments, the modified precursor cell is selected from the group consisting of a bone marrow stem cell, a hematopoietic progenitor cell, and a cord blood stem cell.

In certain embodiments, the subject is conditioned by pre-treatment with radiation.

In certain embodiments, the subject conditioned by pre-treatment with a chemotherapy.

In certain embodiments, the subject is conditioned by pre-treatment with radiation and chemotherapy.

In certain embodiments, the chemotherapy is selected from the group consisting of anti-thymocyte globulin, carmustine, clofarabine, bulsulfan, carboplatin, cyclosporin A, cyclophosphamide, etoposide, fludarabine, melphalan, methotrexate, tacrolimus, thiotepa, topotecan, or any combination thereof.

In certain embodiments, the hematologic malignancy is a myeloid malignancy selected from the group consisting of acute myelogenous leukemia (AML), chronic myelogenous leukemia (CML), myelodysplastic neoplasm, and myeloproliferative neoplasm.

### BRIEF DESCRIPTION OF THE DRAWINGS

The foregoing and other features and advantages of the present invention will be more fully understood from the following detailed description of illustrative embodiments taken in conjunction with the accompanying drawings.

FIG. 1 is a diagram illustrating that individually designed CAR-T cell therapies for each indication is inefficient and impaired by a paucity of suitable target-antigens.

FIG. 2 is a diagram illustrating the expression of CD45 on multiple hematopoietic lineage cells.

FIG. 3 illustrates targeting of the pan-leukocyte antigen CD45 by a single CAR T cell can be used to treat multiple indications.

FIG. 4 depicts previously published data demonstrating the expression of CD45 as being present on all nucleated hematopoietic cells.

FIG. 5 depicts the use of a single CD33-specific CAR T cell in combination with CD33-deleted hematopoietic stem and progenitor cells (HSPCs) for the treatment of CD33-expressing malignancies as disclosed in WO2017079400.

FIG. 6 depicts reduced population doubling in CD45-specific CAR T cells due to fratricide.

FIG. 7 is a diagram illustrating two embodiments of the current invention: the generation of fratricide-resistant CD45 specific CAR T cells and the generation of a CD45 CAR T cell resistant hematopoietic system in patients.

FIGS. 8A-8B depict CD45 specific CAR T cell constructs. FIG. 8A is a diagram of two CAR constructs showing VH/VL and VL/VH arrangement of variable regions while both constructs possess similar transmembrane (TM), 4-1BB/CD3zeta signaling (BBz), P2A, and tNGFR domains. FIG. 8B is a table listing the CD45 CAR T constructs of the current invention. Red boxes highlight the four selected for further development.

FIGS. 9A-9B depict scFv screening for functional CD45-directed CAR T cell constructs.

FIG. 9A is a flowchart detailing the process of screening and validating scFv. FIG. 9B illustrates the knockout of CD45 in Jurkat reporter cells (top) and the expression of CD45 CAR T cell constructs in these cells (bottom).

FIG. 10 is a diagram illustrating the use of a NFAT-GFP reporter assay as a readout for CAR-T cell activation.

FIGS. 11A-11B illustrate the use of the NFAT-GFP reporter assay to identify three lead scFv's. FIG. 11A is a graph showing the activation of 9.4, BC8, and Gap8.3 scFvs. FIG. 11b is a representative micrograph of GFP expression by activated CAR T cells.

FIGS. 12A-12B illustrate that CD45 CARs specifically recognize CD45 on target cells.

FIGS. 13A-13C illustrate that human CD45-directed CAR T cells show impaired ex vivo expansion due to fratricide.

FIGS. 14A-14B illustrate that knocking out CD45 in human primary T cells does not impair ex vivo expansion using anti-CD3/anti-CD28 microbeads.

FIGS. 15A-15C illustrate that CRISPR-Cas9 deletion of CD45 in CAR45 (CD45-specific) T cells can be used to overcome target-driven fratricide.

FIGS. 16A-16B illustrate that epitope masking and/or CAR downregulation during ex vivo expansion in CAR45-expressing CD45+ T cells but not in CD45 knockout cells.

FIGS. 17A-17B illustrate that CAR45 T cell self-enrichment during primary expansion due to epitope masking can act as a “protection” mechanism for CAR T cells.

FIGS. 18A-18C illustrate that CD45 knockout CAR T cells are functional in vitro and have reduced efficacy in vivo compared with CD45-intact CART cells, using CAR19 as a model.

FIG. 19 illustrates that CD45-specific CAR T cells which are CD45-deficient (CD45KO) can specifically kill CD45 positive tumor cells.

FIGS. 20A-20C illustrate that CD45-directed CAR-T cells which are CD45-deficient (CD45KO) can eliminate AML patient tumor cells.

FIG. 21 is a series of graphs illustrating that CD45KO CD45-specific CAR T cells produce and secrete cytokines after target tumor cell co-culture. Graphs depict IFNγ (top left), TNFα (top right), IL-2 (bottom left), and IL-6 (bottom right) production as measured by ELISA.

FIGS. 22A-22B depict that CD45 CAR constructs have in vivo efficacy but optimal function is limited.

FIG. 23 is a diagram illustrating the structure of human CD45 with a box highlighting the domain recognized by the various CAR constructs of the invention.

FIGS. 24A-24B illustrate that CD45 truncation identifies the domain recognized by various CAR constructs.

FIG. 25 is a diagram illustrating the results of epitope mapping studies for each CAR construct of the current invention.

FIG. 26 illustrates that the lack of sequence identity between mouse and human CD45 extracellular domains can be used to develop a more efficient library for alanine-scanning studies.

FIG. 27 illustrates that alanine mutagenesis does not affect protein expression/transport to the cell surface.

FIGS. 28A-28B illustrate that alanine scanning can identify “hot spot” amino acids involved in CD45/CD45 interaction.

FIG. 29 is a series of graphs illustrating that CD45 mutations do not cause large-scale misfolding of CD45 protein.

FIGS. 30A-30C illustrate that alanine scanning can be used to identify key amino acid residues involved in CD45 self-interactions without causing misfolding of CD45 protein.

FIG. 31 illustrates that alanine-mutated target cells are resistant to CAR T cell mediated killing.

FIG. 32 is a diagram illustrating the epitopes of various CD45 CARs mapped onto the crystal structure of human CD45.

FIG. 33 illustrates that single alanine mutants can be used to identify the minimal essential sequence for CAR recognition.

FIGS. 34A-34B illustrate single alanine mutants abrogate 4B2 CAR recognition and activation.

FIGS. 35A-35B illustrate that multiple mutations of CD45 are required to prevent 9.4 CAR recognition and activation.

FIGS. 36A-36B illustrate that double CD45 mutants are required to prevent BC8 CAR recognition and activation.

FIG. 37 is a diagram of a CRISPR/Cas base-editor complex.

FIG. 38 is a diagram illustrating the mechanism of action of base editing.

FIG. 39 is a diagram of based on previously published studies that illustrates Cas9 variants with evolved PAM-recognition that broaden the scope of their targeting abilities.

FIG. 40 is a diagram of the screening strategy using base-editing to abrogate CAR epitopes on CD45.

FIG. 41 illustrates the generation of CAR T cells against a truncated isoform of CD45.

FIGS. 42A-42B illustrate studies demonstrating that in vitro proliferation of CD45 knockout T cells expressing a CD19-specific CAR are also mildly impaired.

FIGS. 43A-43C illustrate that CD45-deleted CAR T cells show comparable initial anti-tumor clearance but impaired long-term tumor control in vivo using a CD19 expressing tumor model.

FIGS. 44A-44C illustrate previously published data demonstrating that loss of CD45 on post-thymic human T cells may be partially compensated for by expression of the functionally redundant phosphatase CD148.

FIG. 45 illustrates that CD148 is expressed by naïve human T cells and expression increases following T cell activation.

FIG. 46 illustrates the knockout of CD148 in primary human T cells.

FIG. 47 illustrates the generation of CD45/CD148 double knockout CAR T cells.

FIG. 48 illustrates that CD45/CD148 double knockout CAR T cells do not proliferate in response to co-culture with target tumor cells.

FIG. 49 illustrates that CD45/CD148 double knockout CAR T cells have impaired cytotoxic capacity as measured in vitro.

FIG. 50 illustrates the that the targeting the pan-leukocyte antigen CD45 with CAR-T cells has the potential as a universal blood cancer cell therapy.

FIGS. 51A-51C illustrate efficient base-editing in primary T cells with minimal bystander edits. In the upper panel of each figure is illustrated the genomic context of three different gRNAs that can be used to change the amino acids at the targeted CD45 epitope (BE8, FIG. 51A, BE13 FIG. 51B, BE17 FIG. 51C). Lower panel: T cells that have been edited were stained with an anti-CD45 antibody (BC8) from which the lead CAR was made from. The three different base edits abolish binding of BC8 indistinguishable from CD45 knockout T cells, suggesting that the antibody no longer binds to the edited CD45. Moreover, when T cells were transduced with the BC8 CAR45, enrichment occurs so that all T cells at the end of expansion are edited.

FIGS. 52A-52B illustrate that epitope base editing disrupts binding of CAR45 and edited cells are enriched when transduced with CAR45. Amplicon sequencing of the targeted locus shows enrichment of edits when T cells were transduced with CAR45 compared to CAR19 demonstrating protection and selection of edited alleles over time.

FIGS. 53A-53B illustrate that highly efficient epitope base editing enables CAR45 expansion. FIG. 53A: Example sanger sequencing chromatogram showing highly efficient editing at the target nucleotide (97%) for BE8. FIG. 53B: Epitope base editing allows for the expansion of CAR45 T cells to the same extend as CD45 knockout cells suggesting that fratricide has been prevented.

FIG. 54 illustrates that epitope editing does not result in a loss of CD45 expression.

FIG. 55 illustrates that base-editing prevents epitope masking/CAR internalization during ex vivo expansion.

FIG. 56 illustrates that base-editing improves viability of expanded CAR45 T cells.

FIG. 57 illustrates that amino acid substitutions installed by base-editing are sufficient to protect cells from CAR-T mediated killing.

FIG. 58 illustrates that epitope-edited CAR45 T cells elicit effector functions upon co-culture with hematologic cancer cell lines and primary cancer cells in vitro.

FIG. 59 illustrates that CAR45 T cells eliminate tumor cells in an AML PDX model.

FIG. 60 illustrates that epitope base-editing is efficient in CD34+ human hematopoietic stem cells.

FIG. 61 illustrates that epitope edited HSCs can proliferate and differentiate in vitro whereas CD45 deleted cells are negatively impacted (CFU).

FIG. 62 illustrates that CD45 base-edited HSCs engraft and replace peripheral blood mononuclear cells.

FIG. 63 illustrates that edited HSCs can differentiate into myeloid cells (CD33+) in vivo and in vitro.

FIG. 64 illustrates that in vitro differentiated myeloid cells from CD45 edited HSCs can phagocytose E. coli bioparticles and RBC's.

FIG. 65 illustrates that CD45 edited myeloid cells can produce ROS through TLR4 activation or pharmacological stimulation.

FIG. 66 illustrates that CD45 edited myeloid cells can produce effector cytokines in response to LPS stimulation.

FIG. 67 illustrates that base-edited, but not CD45KO HSCs can differentiate into T cells in vivo.

FIG. 68 illustrates that CD45 KO impairs CAR19 T cell function while CD45 base-edited T cells remain functional.

FIG. 69 illustrates that base-edited T cells expand in the blood and editing frequency is retained.

FIG. 70 is a diagram illustrating a non-limiting example workflow of base-editing in primary human B cells in vitro.

FIG. 71 illustrates that CD45 base editing does not impair B cell activation/expansion.

FIG. 72 illustrates that edited HSC's can differentiate into B cells in vivo.

FIGS. 73A-73B illustrate that in vivo differentiated B cells from edited HSCs can be activated ex vivo through the BCR, CD40 and TLR4. PBMCs from engrafted mice were isolated 10 weeks post engraftment and stimulated ex vivo with indicated reagents and activation was measured by flow staining for the B cell activation marker CD86. FIG. 73A: Representative flow plots for each condition. FIG. 73B: Quantification of flow plots (n=3). In vivo differentiated, edited, B cells can be activated through the BCR and through CD40/TLR4R mediated stimulation.

FIG. 74 illustrates that BC8 CAR-T cells cross-react with NHP CD45.

FIG. 75 illustrates that editing the BC8 epitope in NHP is technically feasible.

FIGS. 76A-76C illustrate that multiplex base editing CD45 and CCR5 to eliminate latent HIV reservoirs after autoHSCT.

FIGS. 77A-77C illustrate that HIV Env proteins require initial binding to CD4 followed by co-receptor binding (either CCR5 or CXCR4) to enter human cells similarity of rhesus and human CD4 (FIG. 77B) and CCR5 (FIG. 77C) binding to HIV gp120.

FIG. 78 illustrates that in the context of preventing CXCR4 binding, rhesus and human CXCR4 are 98.4% identical.

FIG. 79 illustrates the efficient disruption of CCR5 using CRISPR/Cas nucleases and adenine base editing.

FIGS. 80A-80B illustrate that HIV challenge of CCR5 edited cells demonstrates protection of CCR5 edited cells compared to non-edited cells. FIG. 80A: Non-edited cells were infected with either R5 tropic X4 tropic virus and infection was measured after 6 days by staining for HIV gag. FIG. 80B: CCR5 edited cells were protected from R5 tropic HIV virus infection compared to control cells as measured by a decrease in gag+ cells.

FIG. 81 illustrates that cells with edited CCR5 become enriched during HIV infection, suggesting resistance to infection.

FIG. 82 illustrates that allele frequency of edited CCR5 becomes enriched during HIV infection, suggesting resistance to infection.

FIG. 83 illustrates several exemplary strategies for knocking-out CD33 protein expression using base-editing without the need for double-strand breaks.

### DETAILED DESCRIPTION

The present invention provides compositions and methods of use of modified immune cells or precursors thereof (e.g., modified T cells) comprising a modification in an endogenous gene locus encoding CD45 and an exogenous (e.g., recombinant, transgenic or engineered) chimeric antigen receptor (CAR) specific for CD45. In some embodiments, the modified immune cells are genetically edited such that the expression of CD45 is modified to lack the epitopes of CD45 recognized by the TCR or CAR. These genetically edited modified immune are able to target CD45-expressing cells including those of hematologic malignancies including cancer without being subject to fratricidal or epitope masking effects that would limit their function. In some embodiments, the genetically edited modified immune cells are combined with hematopoietic progenitor cells comprising the same CD45 alterations such that the subject's bone marrow is reconstituted with cells resistant to the CD45-specific CAR T cells. In some embodiments, the CD45 specific cells of the invention are used in bone marrow conditioning regiments to deplete endogenous hematopoietic cell populations in subjects prior to bone marrow transplant. In certain embodiments, the cells comprising the modified CD45 are combined with other CD45-targeting strategies, including but not limited to antibodies including mAB, antigen-binding fragments, and the like, bispecific T cell engaging antibodies (BiTEs), antibody-drug conjugates (ADC), and radio-immunoconjugates among others.

It is to be understood that the methods described in this disclosure are not limited to particular methods and experimental conditions disclosed herein as such methods and conditions may vary. It is also to be understood that the terminology used herein is for the purpose of describing particular embodiments only, and is not intended to be limiting.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by M R Green and J. Sambrook and Harlow et al., Antibodies: A Laboratory Manual, Chapter 14, Cold Spring Harbor Laboratory, Cold Spring Harbor (2013, 2nd edition).

### Definitions

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

As used herein, to “alleviate” a disease means reducing the severity of one or more symptoms of the disease.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen.

Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material derived from the same individual to which it is later to be re-introduced into the individual.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when administered to a mammal, causes a detectable level of immune suppression or tolerance compared to the immune response detected in the absence of the composition of the invention. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly about 10 amino acids and/or sugars in size. Preferably, the epitope is about 4-18 amino acids, more preferably about 5-16 amino acids, and even more most preferably 6-14 amino acids, more preferably about 7-12, and most preferably about 8-10 amino acids. One skilled in the art understands that generally the overall three-dimensional structure, rather than the specific linear sequence of the molecule, is the main criterion of antigenic specificity and therefore distinguishes one epitope from another. Based on the present disclosure, a peptide used in the present invention can be an epitope.

As used herein, the term “exogenous” refers to any material introduced from or produced outside an organism, cell, tissue or system.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immune response” as used herein is defined as a cellular response to an antigen that occurs when lymphocytes identify antigenic molecules as foreign and induce the formation of antibodies and/or activate lymphocytes to remove the antigen.

The term “immunosuppressive” is used herein to refer to reducing overall immune response.

“Insertion/deletion”, commonly abbreviated “indel,” is a type of genetic polymorphism in which a specific nucleotide sequence is present (insertion) or absent (deletion) in a genome.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “knockdown” as used herein refers to a decrease in gene expression of one or more genes.

The term “knockin” as used herein refers to an exogenous nucleic acid sequence that has been inserted into a target sequence (e.g., endogenous gene locus). For example, a modified CD45 knockin into an endogenous CD45 locus refers to a nucleic acid sequence encoding a modified CD45 protein that has been inserted into a target location within the endogenous CD45 gene sequence. In some embodiments, wherein the target sequence is a gene, a knockin is generated resulting in the exogenous nucleic acid sequence being in operable linkage with any upstream and/or downstream regulatory elements controlling expression of the target gene. In some embodiments, the knockin is generated resulting in the exogenous nucleic acid sequence not being in operable linkage with any upstream and/or downstream regulatory elements controlling expression of the target gene.

The term “knockout” as used herein refers to the ablation of gene expression of one or more genes.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “oligonucleotide” typically refers to short polynucleotides. It will be understood that when a nucleotide sequence is represented by a DNA sequence (i.e., A, T, C, G), this also includes an RNA sequence (i.e., A, U, C, G) in which “U” replaces “T.”

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some versions contain an intron(s).

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as upregulation of interferon-gamma, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

A “target site” or “target sequence” refers to a nucleic acid sequence that defines a portion of a nucleic acid to which a binding molecule may specifically bind under conditions sufficient for binding to occur. In some embodiments, a target sequence refers to a genomic nucleic acid sequence that defines a portion of a nucleic acid to which a binding molecule may specifically bind under conditions sufficient for binding to occur.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (α) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “therapeutic” as used herein means a treatment and/or prophylaxis. A therapeutic effect is obtained by suppression, remission, or eradication of a disease state.

“Transplant” refers to a biocompatible lattice or a donor tissue, organ or cell, to be transplanted. An example of a transplant may include but is not limited to skin cells or tissue, bone marrow, and solid organs such as heart, pancreas, kidney, lung and liver. A transplant can also refer to any material that is to be administered to a host. For example, a transplant can refer to a nucleic acid or a protein.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention provides CD45 specific chimeric antigen receptors (CARs; e.g., a CD45 CAR) and modified cells comprising the same. Also provided are compositions and methods for utilizing CD45 specific CARs to treat cancer. In particular, a CD45 CAR of the present invention may be suitable for hematologic malignancies which express CD45 such as lymphomas, myelomas, and the like. It is also contemplated that the CD45 specific CARs of the invention may be used to deplete CD45-expressing cell populations in subjects in need thereof, such as part of a conditioning regimen prior to bone marrow stem cell transplantation or to eliminate a reservoir of HIV-positive cells.

The present invention provides modified immune cells or precursors thereof (e.g., a T cell) comprising a CRISPR-mediated modification in an endogenous gene locus encoding CD45 that is capable of modifying the amino acid sequence of the endogenous CD45, and an exogenous CAR as described herein. The purpose of this downregulation is to avoid CD45 specific toxicity directed against the CD45 specific T cells due to their expression of CD45. In certain embodiments, the modification of CD45 involves altering the amino acids of the endogenous CD45 gene which are epitopes for the CD45 CAR such that the altered CD45 protein is not able to be bound by the CD45 CAR. In certain embodiments, these modifications are done to the CAR T cell itself. In certain embodiments, these modifications are also made to hematopoietic stem cells which are then used to engraft the subject receiving the CD45 CAR such that a hematopoietic system that is resistant to the CD45 CAR can be provided.

### CD45 Protein

CD45 is a prototypic type I transmembrane protein tyrosine phosphatase which, in humans, is the product of the PTPRC gene. Widely expressed on most cells of hematopoietic origin (erythrocytes and plasma cells being notable exceptions), CD45 plays a key role in regulating T- and B-cell antigen receptor signaling via direct interaction with its extracellular domain, activation of Src kinases, and/or suppression of JAK kinases. PTPRC is a relatively large gene consisting of 34 exons, three of which (exons 4, 5, and 6) can be alternately spliced to generate at least eight different isoforms. In mammalian immune cells, CD45 isoform expression closely follows differentiation status. For example, in T cells, CD45RA expression is typical for naïve T lymphocytes, while CD45RO expression is associated with activated and memory T lymphocytes, where it facilitates T cell activation. In certain embodiments of the current invention, the targeting of CD45 by CAR T cells is used to deplete hematopoietic cells and fully differentiated cells of hematopoietic origin. In certain embodiments, the targeting of CD45 is used to preferentially eliminate cancerous or otherwise dysfunctional cells of hematopoietic origin. In certain embodiments, the targeting of CD45 is used to deplete hematopoietic cells in a subject in need thereof as part of a conditioning or treatment regimen.

### Chimeric Antigen Receptor (CAR)

The present invention provides compositions and methods for modified immune cells or precursor cells thereof, e.g., modified T cells, comprising a chimeric antigen receptor (CAR) having affinity for CD45. A subject CAR of the invention comprises an antigen binding domain (e.g., CD45 binding domain), a transmembrane domain, a costimulatory signaling domain, and an intracellular signaling domain. A subject CAR of the invention may optionally comprise a hinge domain. Accordingly, a subject CAR of the invention comprises an antigen binding domain (e.g., CD45 binding domain), a hinge domain, a transmembrane domain, a costimulatory signaling domain, and an intracellular signaling domain. In some embodiments, each of the domains of a subject CAR is separated by a linker.

The antigen binding domain may be operably linked to another domain of the CAR, such as the transmembrane domain, the costimulatory signaling domain or the intracellular signaling domain, each described elsewhere herein, for expression in the cell. In one embodiment, a first nucleic acid sequence encoding the antigen binding domain is operably linked to a second nucleic acid encoding a transmembrane domain, and further operably linked to a third a nucleic acid sequence encoding a costimulatory signaling domain.

The antigen binding domains described herein can be combined with any of the transmembrane domains, any of the costimulatory signaling domains, any of the intracellular signaling domains, or any of the other domains described herein that may be included in a CAR of the present invention.

In one aspect, the invention includes a chimeric antigen receptor (CAR) that specifically binds CD45, comprising: a CD45-specific antigen binding domain, optionally a hinge domain, a transmembrane domain, a costimulatory signaling domain, and an intracellular signaling domain.

In one aspect, the invention includes a chimeric antigen receptor (CAR) that specifically binds CD45, comprising: a CD45-specific antigen binding domain, optionally a hinge domain, a transmembrane domain, a costimulatory signaling domain, and an intracellular signaling domain.

In one exemplary embodiment, the invention includes a chimeric antigen receptor (CAR) that specifically binds CD45, comprising: a CD45-specific antigen binding domain comprising a heavy chain variable (VH) domain and a light chain variable (VL) domain, wherein the VH domain comprises three heavy chain complementarity determining regions, wherein HCDR1 comprises an amino acid sequence selected from the group consisting of SEQ ID NOs: 89, 92, 95, 98, 101, 104, 107, 110, 113, and 116, HCDDR2 comprises an amino acid sequence selected from the group consisting of SEQ ID NOs: 90, 93, 96, 99, 102, 105, 108, 111, 114, and 117, and HCDR3 comprises and amino acid sequence selected from the group comprising SEQ ID NOs: 91, 94, 97, 100, 103, 106, 109, 112, 115, and 118; and wherein the VL domain comprises a light chain variable region that comprises three light chain complementarity determining regions (LCDRs), wherein LCDR1 comprises an amino acid sequence selected from the group consisting of SEQ ID NOs: 119, 122, 125, 128, 131, 134, 137, 140, and 143, LCDR2 comprises and amino acid sequence selected from the group consisting of SEQ ID NOs: 120, 123, 126, 129, 132, 135, 138, 141, and 144, and LCDR3 comprises an amino acid sequence selected from the group consisting of SEQ ID NOs: 121, 124, 127, 130, 133, 136, 139, 142, and 145; a CD8 hinge domain; a CD8 transmembrane domain; a 4-1BB costimulatory signaling domain; and a CD3 zeta intracellular signaling domain.

In some embodiments, a genetically modified immune cell (e.g., T cell) or precursor cell thereof of the present invention comprises a chimeric antigen receptor (CAR) having affinity for CD45. In some embodiments, a genetically modified immune cell (e.g., T cell) or precursor cell thereof of the present invention comprises a chimeric antigen receptor (CAR) having affinity for CD45.

In certain embodiments, the genetically modified cell is a T cell.

Accordingly, in one exemplary embodiment, provided herein is a genetically modified T cell comprising a chimeric antigen receptor (CAR) that specifically binds CD45, comprising: a CD45 specific antigen binding domain, an optional hinge domain, a transmembrane domain, a costimulatory signaling domain, and an intracellular signaling domain.

Accordingly, in one exemplary embodiment, the invention includes a is a genetically modified T cell comprising chimeric antigen receptor (CAR) that specifically binds CD45, comprising: a CD45-specific antigen binding domain comprising a heavy chain variable (VH) domain and a light chain variable (VL) domain, wherein the VH domain comprises three heavy chain complementarity determining regions, wherein HCDR1 comprises an amino acid sequence selected from the group consisting of SEQ ID NOs: 89, 92, 95, 98, 101, 104, 107, 110, 113, and 116, HCDDR2 comprises an amino acid sequence selected from the group consisting of SEQ ID NOs: 90, 93, 96, 99, 102, 105, 108, 111, 114, and 117, and HCDR3 comprises and amino acid sequence selected from the group comprising SEQ ID NOs: 91, 94, 97, 100, 103, 106, 109, 112, 115, and 118; and wherein the VL domain comprises a light chain variable region that comprises three light chain complementarity determining regions (LCDRs), wherein LCDR1 comprises an amino acid sequence selected from the group consisting of SEQ ID NOs: 119, 122, 125, 128, 131, 134, 137, 140, and 143, LCDR2 comprises and amino acid sequence selected from the group consisting of SEQ ID NOs: 120, 123, 126, 129, 132, 135, 138, 141, and 144, and LCDR3 comprises an amino acid sequence selected from the group consisting of SEQ ID NOs: 121, 124, 127, 130, 133, 136, 139, 142, and 145; a CD8 hinge domain; a CD8 transmembrane domain; a 4-1BB costimulatory signaling domain; and a CD3 zeta intracellular signaling domain.

In certain embodiments of the invention, the CAR is encoded by a nucleic acid sequence comprising the nucleotide sequence of SEQ ID NOs: 174, 176, 178, 180, 182, 184, 186, 188, 190, 192, 194, 196, 198, 200, 202, 204, 208, and 210. In certain embodiments of the invention, the CAR comprises the amino acid sequence of SEQ ID NOs: 175, 177, 179, 181, 183, 185, 187, 189, 191, 193, 195, 197, 199, 201, 203, 205, 207, 209, and 211.

Sequences of individual domains and the CAR are found in Table 1.

Accordingly, a subject CAR may be a CAR having affinity for CD45, comprising a CD45 binding domain comprising the amino acid sequence set forth in SEQ ID NOs: 175, 177, 179, 181, 183, 185, 187, 189, 191, 193, 195, 197, 199, 201, 203, 205, 207, 209, and 211. A subject CD45 CAR may further comprise a leader sequence comprising an amino acid sequence set forth in SEQ ID NO: 166. A subject CD45 CAR may further comprise a hinge domain comprising an amino acid sequence set forth in SEQ ID NO: 168. A subject CD45 CAR may further comprise a transmembrane domain comprising an amino acid sequence set forth in SEQ ID NO: 169. A subject CD45 CAR may further comprise a costimulatory signaling domain comprising an amino acid sequence set forth in SEQ ID NO: 170. A subject CD45 CAR may further comprise an intracellular signaling domain comprising an amino acid sequence set forth in SEQ ID NO: 171. A subject Tn-CD45 CAR may comprise an amino acid sequence set forth in SEQ ID NOs: 175, 177, 179, 181, 183, 185, 187, 189, 191, 193, 195, 197, 199, 201, 203, 205, 207, 209, and 211.

### Antigen Binding Domain

The antigen binding domain of a CAR is an extracellular region of the CAR for binding to a specific target antigen including proteins, carbohydrates, and glycolipids. In some embodiments, the CAR comprises affinity to a target antigen (e.g. a tumor associated antigen) on a target cell (e.g. a cancer cell). The target antigen may include any type of protein, or epitope thereof, associated with the target cell. For example, the CAR may comprise affinity to a target antigen on a target cell that indicates a particular status of the target cell.

In certain embodiments, the CAR of the invention comprises an antigen binding domain that binds to CD45. In certain embodiments, the antigen binding domain of the invention comprises an antibody or fragment thereof, that binds to a CD45 molecule. In certain exemplary embodiments, the antigen binding domain is an scFv antibody that binds to CD45. The choice of antigen binding domain depends upon the type and number of antigens that are present on the surface of a target cell. For example, the antigen binding domain may be chosen to recognize an antigen that acts as a cell surface marker on a target cell associated with a particular status of the target cell.

As described herein, a CAR of the present disclosure having affinity for a specific target antigen on a target cell may comprise a target-specific binding domain. In some embodiments, the target-specific binding domain is a human target-specific binding domain, e.g., the target-specific binding domain is of human origin. In an exemplary embodiment, a CAR of the present disclosure having affinity for CD45 on a target cell may comprise a CD45 binding domain. In some embodiments, the CD45 binding domain is a murine CD45 binding domain, e.g., the CD45 binding domain is of murine origin. In some embodiments, the CD45 binding domain is a humanized CD45 binding domain. In some embodiments, the CD45 binding domain is a human CD45 binding domain, e.g., the CD45 binding domain is of human origin.

In some embodiments, the CD45 binding domain is derived from the 4B2 antibody disclosed in U.S. patent application Ser. No. 16/298,202, the disclosure of which is incorporated herein by reference in its entirety. Accordingly, a CAR of the present disclosure comprises a CD45 binding domain derived from the 4B2 antibody disclosed in U.S. patent application Ser. No. 16/298,202.

In some embodiments, the CD45 binding domain is derived from the 9.4 antibody disclosed in PCT application WO2017/009473, the disclosure of which is incorporated herein by reference in its entirety. Accordingly, a CAR of the present disclosure comprises a CD45 binding domain derived from the 9.4 antibody disclosed PCT application WO2017/009473.

In some embodiments, the CD45 binding domain is derived from the CN1 antibody disclosed in Chinese patent application CN106046163, the disclosure of which is incorporated herein by reference in its entirety. Accordingly, a CAR of the present disclosure comprises a CD45 binding domain derived from the CN1 antibody disclosed in Chinese patent application CN106046163.

In some embodiments, the CD45 binding domain is derived from the mAb104 antibody disclosed in PCT application WO2020/092655, the disclosure of which is incorporated herein by reference in its entirety. Accordingly, a CAR of the present disclosure comprises a CD45 binding domain derived from the mAb104 antibody disclosed in Chinese patent application in PCT application WO2020/092655.

In some embodiments, the CD45 binding domain is derived from the NOV45 antibody disclosed in PCT application WO2005/026210, the disclosure of which is incorporated herein by reference in its entirety. Accordingly, a CAR of the present disclosure comprises a CD45 binding domain derived from the NOV45 antibody disclosed in Chinese patent application in PCT application WO2005/026210.

The antigen binding domain can include any domain that binds to the antigen and may include, but is not limited to, a monoclonal antibody, a polyclonal antibody, a synthetic antibody, a human antibody, a humanized antibody, a non-human antibody, and any fragment thereof. Thus, in one embodiment, the antigen binding domain portion comprises a mammalian antibody or a fragment thereof. In another embodiment, the antigen binding domain of the CAR is selected from the group consisting of an anti-CD45 antibody or a fragment thereof. In some embodiments, the antigen binding domain is selected from the group consisting of an antibody, an antigen binding fragment (Fab), and a single-chain variable fragment (scFv). In some embodiments, a CD45 binding domain of the present invention is selected from the group consisting of a CD45-specific antibody, a CD45-specific Fab, and a CD45-specific scFv. In one embodiment, a CD45 binding domain is a CD45-specific antibody. In one embodiment, a CD45 binding domain is a CD45-specific Fab. In one embodiment, a CD45 binding domain is a CD45-specific scFv.

As used herein, the term “single-chain variable fragment” or “scFv” is a fusion protein of the variable regions of the heavy (VH) and light chains (VL) of an immunoglobulin (e.g., mouse or human) covalently linked to form a VH:VL heterodimer. The heavy (VH) and light chains (VL) are either joined directly or joined by a peptide-encoding linker or spacer, which connects the N-terminus of the VH with the C-terminus of the VL, or the C-terminus of the VH with the N-terminus of the VL. The terms “linker” and “spacer” are used interchangeably herein. In some embodiments, the antigen binding domain (e.g., CD45 binding domain) comprises an scFv having the configuration from N-terminus to C-terminus, VH-linker-VL. In some embodiments, the antigen binding domain (e.g., CD45 binding domain) comprises an scFv having the configuration from N-terminus to C-terminus, VL-linker-VH. Those of skill in the art would be able to select the appropriate configuration for use in the present invention.

The linker is typically rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. Various linker sequences are known in the art, including, without limitation, glycine serine (GS) linkers such as (GS)ₙ, (GSGGS)ₙ (SEQ ID NO: 259), (GGGS)ₙ (SEQ ID NO: 260), and (GGGGS)ₙ (SEQ ID NO: 261), where n represents an integer of at least 1. Exemplary linker sequences can comprise amino acid sequences including, without limitation, GGSG (SEQ ID NO: 262), GGSGG (SEQ ID NO: 263), GSGSG (SEQ ID NO: 264), GSGGG (SEQ ID NO: 265), GGGSG (SEQ ID NO: 266), GSSSG (SEQ ID NO: 267), GGGGS (SEQ ID NO: 268), GGGGSGGGGSGGGGS (SEQ ID NO: 269) and the like. Those of skill in the art would be able to select the appropriate linker sequence for use in the present invention. In one embodiment, an antigen binding domain (e.g., CD45 binding domain) of the present invention comprises a heavy chain variable region (VH) and a light chain variable region (VL), wherein the VH and VL is separated by the linker sequence having the amino acid sequence GGGGSGGGGSGGGGS (SEQ ID NO: 269), which may be encoded by a nucleic acid sequence comprising the nucleotide sequence ggtggcggtggctcgggcggtggtgggtcgggt ggcggcggatct (SEQ ID NO: 270).

Despite removal of the constant regions and the introduction of a linker, scFv proteins retain the specificity of the original immunoglobulin. Single chain Fv polypeptide antibodies can be expressed from a nucleic acid comprising VH- and VL-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hybridoma (Larchmt) 2008 27(6):455-51; Peter et al., J Cachexia Sarcopenia Muscle 2012 Aug. 12; Shieh et al., J Imunol 2009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Biol Chem 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

As used herein, “Fab” refers to a fragment of an antibody structure that binds to an antigen but is monovalent and does not have a Fc portion, for example, an antibody digested by the enzyme papain yields two Fab fragments and an Fc fragment (e.g., a heavy (H) chain constant region; Fc region that does not bind to an antigen).

As used herein, “F(ab′)2” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)2” fragment can be split into two individual Fab′ fragments.

In some instances, the antigen binding domain may be derived from the same species in which the CAR will ultimately be used. For example, for use in humans, the antigen binding domain of the CAR may comprise a human antibody as described elsewhere herein, or a fragment thereof.

In one embodiment, the CD45 binding domain comprises a light chain variable region comprising an amino acid sequence set forth in SEQ ID NOs: 156-165. The light chain variable region of the CD45 binding domain comprises three light chain complementarity-determining regions (CDRs). As used herein, a “complementarity-determining region” or “CDR” refers to a region of the variable chain of an antigen binding molecule that binds to a specific antigen. Accordingly, a CD45 binding domain may comprise a light chain variable region that comprises an amino acid sequence selected from the group consisting of SEQ ID NOs: 119, 122, 125, 128, 131, 134, 137, 140, and 143, LCDR2 comprises and amino acid sequence selected from the group consisting of SEQ ID NOs: 120, 123, 126, 129, 132, 135, 138, 141, and 144, and LCDR3 comprises an amino acid sequence selected from the group consisting of SEQ ID NOs: 121, 124, 127, 130, 133, 136, 139, 142, and 145.

In one embodiment, the CD45 binding domain comprises a heavy chain variable region comprising an amino acid sequence set forth in SEQ ID NOs: 156-165. A CD45 binding domain may comprise a heavy chain variable region that comprises a CDR1 comprising an amino acid sequence selected from the group consisting of SEQ ID NOs: 89, 92, 95, 98, 101, 104, 107, 110, 113, and 116, HCDDR2 comprises an amino acid sequence selected from the group consisting of SEQ ID NOs: 90, 93, 96, 99, 102, 105, 108, 111, 114, and 117, and HCDR3 comprises and amino acid sequence selected from the group comprising SEQ ID NOs: 91, 94, 97, 100, 103, 106, 109, 112, 115, and 118.

Tolerable variations of the CD45 binding domain will be known to those of skill in the art, while maintaining specific binding to CD45. For example, in some embodiments the CD45 binding domain comprises an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the amino acid sequences set forth in SEQ ID NOs: 146-155 and 156-165.

In some embodiments, the CD45 binding domain is encoded by a nucleic acid sequence comprising the nucleotide sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to the nucleotide sequence set forth in SEQ ID NOs: 174, 176, 178, 180, 182, 184, 186, 188, 190, 192, 194, 196, 198, 200, 202, 204, 208, and 210.

The antigen binding domain may be operably linked to another domain of the CAR, such as the transmembrane domain or the costimulatory signaling domain, both described elsewhere herein. In one embodiment, a nucleic acid encoding the antigen binding domain is operably linked to a nucleic acid encoding a transmembrane domain and a nucleic acid encoding a costimulatory signaling domain.

The antigen binding domains described herein, such as the antibody or fragment thereof that binds to CD45, can be combined with any of the transmembrane domains described herein, any of the intracellular domains or cytoplasmic domains described herein, or any of the other domains described herein that may be included in the CAR.

### Transmembrane Domain

With respect to the transmembrane domain, the CAR of the present invention (e.g., CD45 CAR) can be designed to comprise a transmembrane domain that connects the antigen binding domain of the CAR to the intracellular domain. The transmembrane domain of a subject CAR is a region that is capable of spanning the plasma membrane of a cell (e.g., an immune cell or precursor thereof). The transmembrane domain is for insertion into a cell membrane, e.g., a eukaryotic cell membrane. In some embodiments, the transmembrane domain is interposed between the antigen binding domain and the intracellular domain of a CAR.

In one embodiment, the transmembrane domain is naturally associated with one or more of the domains in the CAR. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein, e.g., a Type I transmembrane protein. Where the source is synthetic, the transmembrane domain may be any artificial sequence that facilitates insertion of the CAR into a cell membrane, e.g., an artificial hydrophobic sequence. Examples of the transmembrane regions of particular use in this invention include, without limitation, transmembrane domains derived from (i.e., comprise at least the transmembrane region(s) of) the alpha, beta or zeta chain of the T-cell receptor, CD28, CD2, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, and TLR9. In some embodiments, the transmembrane domain may be synthetic, in which case it will comprise predominantly hydrophobic residues such as leucine and valine. In certain exemplary embodiments, a triplet of phenylalanine, tryptophan and valine will be found at each end of a synthetic transmembrane domain.

The transmembrane domains described herein can be combined with any of the antigen binding domains described herein, any of the costimulatory signaling domains described herein, any of the intracellular signaling domains described herein, or any of the other domains described herein that may be included in a subject CAR.

In some embodiments, the transmembrane domain further comprises a hinge region. A subject CAR of the present invention may also include a hinge region. The hinge region of the CAR is a hydrophilic region which is located between the antigen binding domain and the transmembrane domain. In some embodiments, this domain facilitates proper protein folding for the CAR. The hinge region is an optional component for the CAR. The hinge region may include a domain selected from Fc fragments of antibodies, hinge regions of antibodies, CH2 regions of antibodies, CH3 regions of antibodies, artificial hinge sequences or combinations thereof. Examples of hinge regions include, without limitation, a CD8a hinge, artificial hinges made of polypeptides which may be as small as, three glycines (Gly), as well as CH1 and CH3 domains of IgGs (such as human IgG4).

In some embodiments, a subject CAR of the present disclosure includes a hinge region that connects the antigen binding domain with the transmembrane domain, which, in turn, connects to the intracellular domain. The hinge region is preferably capable of supporting the antigen binding domain to recognize and bind to the target antigen on the target cells (see, e.g., Hudecek et al., Cancer Immunol. Res. (2015) 3(2): 125-135). In some embodiments, the hinge region is a flexible domain, thus allowing the antigen binding domain to have a structure to optimally recognize the specific structure and density of the target antigens on a cell such as tumor cell. The flexibility of the hinge region permits the hinge region to adopt many different conformations.

In some embodiments, the hinge region is an immunoglobulin heavy chain hinge region. In some embodiments, the hinge region is a hinge region polypeptide derived from a receptor (e.g., a CD8-derived hinge region).

The hinge region can have a length of from about 4 amino acids to about 50 amino acids, e.g., from about 4 amino acids to about 10 amino acids, from about 10 amino acids to about 15 amino acids, from about 15 amino acids to about 20 amino acids, from about 20 amino acids to about 25 amino acids, from about 25 amino acids to about 30 amino acids, from about 30 amino acids to about 40 amino acids, or from about 40 amino acids to about 50 amino acids.

Suitable hinge regions can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids.

For example, hinge regions include glycine polymers (G)ₙ, glycine-serine polymers (including, for example, (GS)ₙ, (GSGGS)ₙ (SEQ ID NO: 259) and (GGGS)ₙ (SEQ ID NO: 260), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see, e.g., Scheraga, Rev. Computational. Chem. (1992) 2: 73-142). Exemplary hinge regions can comprise amino acid sequences including, but not limited to, GGSG (SEQ ID NO: 261), GGSGG (SEQ ID NO: 262), GSGSG (SEQ ID NO: 263), GSGGG (SEQ ID NO: 264), GGGSG (SEQ ID NO: 265), GSSSG (SEQ ID NO: 266), and the like.

In some embodiments, the hinge region is an immunoglobulin heavy chain hinge region. Immunoglobulin hinge region amino acid sequences are known in the art; see, e.g., Tan et al., Proc. Natl. Acad. Sci. USA (1990) 87(1):162-166; and Huck et al., Nucleic Acids Res. (1986) 14(4): 1779-1789. As non-limiting examples, an immunoglobulin hinge region can include one of the following amino acid sequences: DKTHT (SEQ ID NO: 271); CPPC (SEQ ID NO: 272); CPEPKSCDTPPPCPR (SEQ ID NO: 273) (see, e.g., Glaser et al., J. Biol. Chem. (2005) 280:41494-41503); ELKTPLGDTTHT (SEQ ID NO: 274); KSCDKTHTCP (SEQ ID NO: 275); KCCVDCP (SEQ ID NO: 276); KYGPPCP (SEQ ID NO: 277); EPKSCDKTHTCPPCP (SEQ ID NO: 278) (human IgG1 hinge); ERKCCVECPPCP (SEQ ID NO: 279) (human IgG2 hinge); ELKTPLGDTTHTCPRCP (SEQ ID NO: 280) (human IgG3 hinge); SPNMVPHAHHAQ (SEQ ID NO: 281) (human IgG4 hinge); and the like.

The hinge region can comprise an amino acid sequence of a human IgG1, IgG2, IgG3, or IgG4, hinge region. In one embodiment, the hinge region can include one or more amino acid substitutions and/or insertions and/or deletions compared to a wild-type (naturally-occurring) hinge region. For example, His229 of human IgG1 hinge can be substituted with Tyr, so that the hinge region comprises the sequence EPKSCDKTYTCPPCP (SEQ ID NO: 278); see, e.g., Yan et al., J. Biol. Chem. (2012) 287: 5891-5897. In one embodiment, the hinge region can comprise an amino acid sequence derived from human CD8, or a variant thereof.

In one embodiment, the transmembrane domain comprises a CD8α transmembrane domain. In some embodiments, a subject CAR comprises a CD8α transmembrane domain comprising the amino acid sequence set forth in SEQ ID NO: 169.

In another embodiment, a subject CAR comprises a CD8α hinge domain and a CD8α transmembrane domain. In one embodiment, the CD8α hinge domain comprises the amino acid sequence set forth in SEQ ID NO: 168.

Tolerable variations of the transmembrane and/or hinge domain will be known to those of skill in the art, while maintaining its intended function. For example, in some embodiments a transmembrane domain or hinge domain comprises an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the amino acid sequences set forth in SEQ ID NOs: 168 and 169.

The transmembrane domain may be combined with any hinge domain and/or may comprise one or more transmembrane domains described herein.

The transmembrane domains described herein, such as a transmembrane region of alpha, beta or zeta chain of the T-cell receptor, CD28, CD2, CD3 epsilon, CD45, CD4, CD5, CD7, CD8, CD9, CD 16, CD22, CD33, CD37, CD64, CD80, CD86, CD134 (OX-40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, and TLR9, can be combined with any of the antigen binding domains described herein, any of the costimulatory signaling domains or intracellular domains or cytoplasmic domains described herein, or any of the other domains described herein that may be included in the CAR.

In one embodiment, the transmembrane domain may be synthetic, in which case it will comprise predominantly hydrophobic residues such as leucine and valine. In exemplary embodiments, a triplet of phenylalanine, tryptophan and valine will be found at each end of a synthetic transmembrane domain.

In some embodiments, a subject CAR may further comprise, between the extracellular domain and the transmembrane domain of the CAR, or between the intracellular domain and the transmembrane domain of the CAR, a spacer domain. As used herein, the term “spacer domain” generally means any oligo- or polypeptide that functions to link the transmembrane domain to, either the extracellular domain or, the intracellular domain in the polypeptide chain. A spacer domain may comprise up to 300 amino acids, e.g., 10 to 100 amino acids, or 25 to 50 amino acids. In some embodiments, the spacer domain may be a short oligo- or polypeptide linker, e.g., between 2 and 10 amino acids in length. For example, glycine-serine doublet provides a particularly suitable linker between the transmembrane domain and the intracellular signaling domain of the subject CAR.

Accordingly, a subject CAR of the present disclosure may comprise any of the transmembrane domains, hinge domains, or spacer domains described herein.

### Intracellular Domain

A subject CAR of the present invention also includes an intracellular domain. The intracellular domain of the CAR is responsible for activation of at least one of the effector functions of the cell in which the CAR is expressed (e.g., immune cell). The intracellular domain transduces the effector function signal and directs the cell (e.g., immune cell) to perform its specialized function, e.g., harming and/or destroying a target cell.

The intracellular domain or otherwise the cytoplasmic domain of the CAR is responsible for activation of the cell in which the CAR is expressed. Examples of an intracellular domain for use in the invention include, but are not limited to, the cytoplasmic portion of a surface receptor, co-stimulatory molecule, and any molecule that acts in concert to initiate signal transduction in the T cell, as well as any derivative or variant of these elements and any synthetic sequence that has the same functional capability.

In certain embodiments, the intracellular domain comprises a costimulatory signaling domain. In certain embodiments, the intracellular domain comprises an intracellular signaling domain. In certain embodiments, the intracellular domain comprises a costimulatory signaling domain and an intracellular signaling domain. In certain embodiments, the intracellular domain comprises 4-1BB and CD3 zeta. In certain embodiments, the costimulatory signaling domain comprises 4-1BB. In certain embodiments, the intracellular signaling domain comprises CD3 zeta.

In one embodiment, the intracellular domain of the CAR comprises a costimulatory signaling domain which includes any portion of one or more co-stimulatory molecules, such as at least one signaling domain from CD2, CD3, CD8, CD27, CD28, OX40, ICOS, 4-1BB, PD-1, any derivative or variant thereof, any synthetic sequence thereof that has the same functional capability, and any combination thereof.

Examples of the intracellular signaling domain include, without limitation, the ζ chain of the T cell receptor complex or any of its homologs, e.g., η chain, FcsRIγ and β chains, MB 1 (Iga) chain, B29 (Ig) chain, etc., human CD3 zeta chain, CD3 polypeptides (Δ, δ and ε), syk family tyrosine kinases (Syk, ZAP 70, etc.), src family tyrosine kinases (Lck, Fyn, Lyn, etc.), and other molecules involved in T cell transduction, such as CD2, CD5 and CD28. In one embodiment, the intracellular signaling domain may be human CD3 zeta chain, FcyRIII, FcsRI, cytoplasmic tails of Fc receptors, an immunoreceptor tyrosine-based activation motif (ITAM) bearing cytoplasmic receptors, and combinations thereof.

Other examples of the intracellular domain include a fragment or domain from one or more molecules or receptors including, but are not limited to, TCR, CD3 zeta, CD3 gamma, CD3 delta, CD3 epsilon, CD86, common FcR gamma, FcR beta (Fc Epsilon Rib), CD79a, CD79b, Fc gamma R11a, DAP10, DAP12, T cell receptor (TCR), CD8, CD27, CD28, 4-1BB (CD137), OX9, OX40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CD5, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD1Id, ITGAE, CD103, ITGAL, CD11a, LFA-1, ITGAM, CD lib, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAMI, CRTAM, Ly9 (CD229), CD160 (BY55), PSGL1, CD100 (SEMA4D), CD69, SLAMF6 (NTB-A, Ly108), SLAM (SLAMFI, CD150, IPO-3), BLAME (SLAMF8), SELPLG (CD 162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

Additional examples of intracellular domains include, without limitation, intracellular signaling domains of several types of various other immune signaling receptors, including, but not limited to, first, second, and third generation T cell signaling proteins including CD3, B7 family costimulatory, and Tumor Necrosis Factor Receptor (TNFR) superfamily receptors (see, e.g., Park and Brentjens, J. Clin. Oncol. (2015) 33(6): 651-653). Additionally, intracellular signaling domains may include signaling domains used by NK and NKT cells (see, e.g., Hermanson and Kaufman, Front. Immunol. (2015) 6: 195) such as signaling domains of NKp30 (B7-H6) (see, e.g., Zhang et al., J. Immunol. (2012) 189(5): 2290-2299), and DAP 12 (see, e.g., Topfer et al., J. Immunol. (2015) 194(7): 3201-3212), NKG2D, NKp44, NKp46, DAP10, and CD3z.

Intracellular signaling domains suitable for use in a subject CAR of the present invention include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation of the CAR (i.e., activated by antigen and dimerizing agent). In some embodiments, the intracellular signaling domain includes at least one (e.g., one, two, three, four, five, six, etc.) ITAM motifs as described below. In some embodiments, the intracellular signaling domain includes DAP10/CD28 type signaling chains. In some embodiments, the intracellular signaling domain is not covalently attached to the membrane bound CAR, but is instead diffused in the cytoplasm.

Intracellular signaling domains suitable for use in a subject CAR of the present invention include immunoreceptor tyrosine-based activation motif (ITAM)-containing intracellular signaling polypeptides. In some embodiments, an ITAM motif is repeated twice in an intracellular signaling domain, where the first and second instances of the ITAM motif are separated from one another by 6 to 8 amino acids. In one embodiment, the intracellular signaling domain of a subject CAR comprises 3 ITAM motifs. In some embodiments, intracellular signaling domains includes the signaling domains of human immunoglobulin receptors that contain immunoreceptor tyrosine based activation motifs (ITAMs) such as, but not limited to, Fc gamma RI, Fc gamma RIIA, Fc gamma RIIC, Fc gamma RIIIA, FcRL5 (see, e.g., Gillis et al., Front. (2014) Immunol. 5:254).

A suitable intracellular signaling domain can be an ITAM motif-containing portion that is derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12, FCER1G (Fc epsilon receptor I gamma chain), CD3D (CD3 delta), CD3E (CD3 epsilon), CD3G (CD3 gamma), CD3Z (CD3 zeta), and CD79A (antigen receptor complex-associated protein alpha chain).

In one embodiment, the intracellular signaling domain is derived from DAP12 (also known as TYROBP; TYRO protein tyrosine kinase binding protein; KARAP; PLOSL; DNAX-activation protein 12; KAR-associated protein; TYRO protein tyrosine kinase-binding protein; killer activating receptor associated protein; killer-activating receptor-associated protein; etc.). In one embodiment, the intracellular signaling domain is derived from FCER1G (also known as FCRG; Fe epsilon receptor I gamma chain; Fc receptor gamma-chain; fc-epsilon RI-gamma; fcR gamma; fceR1 gamma; high affinity immunoglobulin epsilon receptor subunit gamma; immunoglobulin E receptor, high affinity, gamma chain; etc.). In one embodiment, the intracellular signaling domain is derived from T-cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T-cell receptor T3 delta chain; T-cell surface glycoprotein CD3 delta chain; etc.). In one embodiment, the intracellular signaling domain is derived from T-cell surface glycoprotein CD3 epsilon chain (also known as CD3e, T-cell surface antigen T3/Leu-4 epsilon chain, T-cell surface glycoprotein CD3 epsilon chain, AI504783, CD3, CD3epsilon, T3e, etc.). In one embodiment, the intracellular signaling domain is derived from T-cell surface glycoprotein CD3 gamma chain (also known as CD3G, T-cell receptor T3 gamma chain, CD3-GAMMA, T3G, gamma polypeptide (TiT3 complex), etc.). In one embodiment, the intracellular signaling domain is derived from T-cell surface glycoprotein CD3 zeta chain (also known as CD3Z, T-cell receptor T3 zeta chain, CD247, CD3-ZETA, CD3H, CD3Q, T3Z, TCRZ, etc.). In one embodiment, the intracellular signaling domain is derived from CD79A (also known as B-cell antigen receptor complex-associated protein alpha chain; CD79a antigen (immunoglobulin-associated alpha); MB-1 membrane glycoprotein; Ig-alpha; membrane-bound immunoglobulin-associated protein; surface IgM-associated protein; etc.). In one embodiment, an intracellular signaling domain suitable for use in a subject CAR of the present disclosure includes a DAP10/CD28 type signaling chain. In one embodiment, an intracellular signaling domain suitable for use in a subject CAR of the present disclosure includes a ZAP70 polypeptide. In some embodiments, the intracellular signaling domain includes a cytoplasmic signaling domain of TCR zeta, FcR gamma, FcR beta, CD3 gamma, CD3 delta, CD3 epsilon, CD5, CD22, CD79a, CD79b, or CD66d. In one embodiment, the intracellular signaling domain in the CAR includes a cytoplasmic signaling domain of human CD3 zeta.

While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The intracellular signaling domain includes any truncated portion of the intracellular signaling domain sufficient to transduce the effector function signal.

The intracellular signaling domains described herein can be combined with any of the costimulatory signaling domains described herein, any of the antigen binding domains described herein, any of the transmembrane domains described herein, or any of the other domains described herein that may be included in the CAR.

In one embodiment, the intracellular domain of a subject CAR comprises a 4-1BB costimulatory domain comprising the amino acid sequence set forth in SEQ ID NO: 170.

In one embodiment, the intracellular domain of a subject CAR comprises a CD3 zeta intracellular signaling domain comprising the amino acid sequence set forth in SEQ ID NO: 171.

Tolerable variations of the intracellular domain will be known to those of skill in the art, while maintaining specific activity. For example, in some embodiments the intracellular domain comprises an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the amino acid sequences set forth in SEQ ID NOs: 170 and 171.

In one exemplary embodiment, the intracellular domain of a subject CAR comprises a 4-1BB costimulatory domain and a CD3 zeta intracellular signaling domain.

### Modified Immune Cells

The present invention provides a modified immune cell or precursor cell thereof (e.g., a modified T cell, a modified NK cell, a modified NKT cell), comprising a subject CAR. Accordingly, such modified cells possess the specificity directed by the CAR that is expressed therein. For example, a modified cell of the present invention comprising a CD45 CAR possesses specificity for CD45 on a target cell.

Any modified cell comprising a CAR comprising any antigen binding domain, any hinge, any transmembrane domain, any intracellular costimulatory domain, and any intracellular signaling domain described herein is envisioned, and can readily be understood and made by a person of skill in the art in view of the disclosure herein.

In some embodiments, the modified cell is an immune cell or precursor cell thereof. In an exemplary embodiment, the modified cell is a T cell. In an exemplary embodiment, the modified cell is an autologous cell. In an exemplary embodiment, the modified cell is an autologous immune cell or precursor cell thereof. In an exemplary embodiment, the modified cell is an autologous T cell.

### Nucleic Acids and Expression Vectors

The present invention provides a nucleic acid encoding a CAR having affinity for CD45 (e.g. CD45). As described herein, a subject CAR comprises an antigen binding domain (e.g., CD45 binding domain), a transmembrane domain, and an intracellular domain. Accordingly, the present invention provides a nucleic acid encoding an antigen binding domain (e.g., CD45 binding domain), a transmembrane domain, and an intracellular domain of a subject CAR.

In an exemplary embodiment, the antigen binding domain of the CD45 CAR of the present invention is encoded by any one of the nucleotide sequences set forth in SEQ ID NOs: 174, 176, 178, 180, 182, 184, 186, 188, 190, 192, 194, 196, 198, 200, 202, 204, 208, and 210.

In some embodiments, a nucleic acid of the present disclosure may be operably linked to a transcriptional control element, e.g., a promoter, and enhancer, etc. Suitable promoter and enhancer elements are known to those of skill in the art.

For expression in a bacterial cell, suitable promoters include, but are not limited to, lacI, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (A1cR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

In some embodiments, the promoter is a CD8 cell-specific promoter, a CD4 cell-specific promoter, a neutrophil-specific promoter, or an NK-specific promoter. For example, a CD4 gene promoter can be used; see, e.g., Salmon et al. Proc. Natl. Acad. Sci. USA (1993) 90:7739; and Marodon et al. (2003) Blood 101:3416. As another example, a CD8 gene promoter can be used. NK cell-specific expression can be achieved by use of an NcrI (p46) promoter; see, e.g., Eckelhart et al. Blood (2011) 117:1565.

For expression in a yeast cell, a suitable promoter is a constitutive promoter such as an ADH1 promoter, a PGK1 promoter, an ENO promoter, a PYK1 promoter and the like; or a regulatable promoter such as a GAL1 promoter, a GAL10 promoter, an ADH2 promoter, a PHOS promoter, a CUP1 promoter, a GALT promoter, a MET25 promoter, a MET3 promoter, a CYC1 promoter, a HIS3 promoter, an ADH1 promoter, a PGK promoter, a GAPDH promoter, an ADC1 promoter, a TRP1 promoter, a URA3 promoter, a LEU2 promoter, an ENO promoter, a TP1 promoter, and AOX1 (e.g., for use in Pichia). Selection of the appropriate vector and promoter is well within the level of ordinary skill in the art. Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; in vivo regulated promoters, such as an ssaG promoter or a related promoter (see, e.g., U.S. Patent Publication No. 20040131637), a pagC promoter (Pulkkinen and Miller, J. Bacteriol. (1991) 173(1): 86-93; Alpuche-Aranda et al., Proc. Natl. Acad. Sci. USA (1992) 89(21): 10079-83), a nirB promoter (Harborne et al. Mol. Micro. (1992) 6:2805-2813), and the like (see, e.g., Dunstan et al., Infect. Immun. (1999) 67:5133-5141; McKelvie et al., Vaccine (2004) 22:3243-3255; and Chatfield et al., Biotechnol. (1992) 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spy promoter, and the like; a promoter derived from the pathogenicity island SPI-2 (see, e.g., WO96/17951); an actA promoter (see, e.g., Shetron-Rama et al., Infect. Immun. (2002) 70:1087-1096); an rpsM promoter (see, e.g., Valdivia and Falkow Mol. Microbiol. (1996). 22:367); a tet promoter (see, e.g., Hillen, W. and Wissmann, A. (1989) In Saenger, W. and Heinemann, U. (eds), Topics in Molecular and Structural Biology, Protein—Nucleic Acid Interaction. Macmillan, London, UK, Vol. 10, pp. 143-162); an SP6 promoter (see, e.g., Melton et al., Nucl. Acids Res. (1984) 12:7035); and the like. Suitable strong promoters for use in prokaryotes such as Escherichia coli include, but are not limited to Trc, Tac, T5, T7, and P Lambda. Non-limiting examples of operators for use in bacterial host cells include a lactose promoter operator (LacI repressor protein changes conformation when contacted with lactose, thereby preventing the Lad repressor protein from binding to the operator), a tryptophan promoter operator (when complexed with tryptophan, TrpR repressor protein has a conformation that binds the operator; in the absence of tryptophan, the TrpR repressor protein has a conformation that does not bind to the operator), and a tac promoter operator (see, e.g., deBoer et al., Proc. Natl. Acad. Sci. U.S.A. (1983) 80:21-25).

Other examples of suitable promoters include the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, the EF-1 alpha promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the locus or construct or transgene containing the suitable promoter is irreversibly switched through the induction of an inducible system. Suitable systems for induction of an irreversible switch are well known in the art, e.g., induction of an irreversible switch may make use of a Cre-lox-mediated recombination (see, e.g., Fuhrmann-Benzakein, et al., Proc. Natd. Acad. Sci. USA (2000) 28:e99, the disclosure of which is incorporated herein by reference). Any suitable combination of recombinase, endonuclease, ligase, recombination sites, etc. known to the art may be used in generating an irreversibly switchable promoter. Methods, mechanisms, and requirements for performing site-specific recombination, described elsewhere herein, find use in generating irreversibly switched promoters and are well known in the art, see, e.g., Grindley et al. Annual Review of Biochemistry (2006) 567-605; and Tropp, Molecular Biology (2012) (Jones & Bartlett Publishers, Sudbury, MA), the disclosures of which are incorporated herein by reference.

In some embodiments, a nucleic acid of the present disclosure further comprises a nucleic acid sequence encoding a CAR inducible expression cassette. In one embodiment, the CAR inducible expression cassette is for the production of a transgenic polypeptide product that is released upon CAR signaling. See, e.g., Chmielewski and Abken, Expert Opin. Biol. Ther. (2015) 15(8): 1145-1154; and Abken, Immunotherapy (2015) 7(5): 535-544.

A nucleic acid of the present disclosure may be present within an expression vector and/or a cloning vector. An expression vector can include a selectable marker, an origin of replication, and other features that provide for replication and/or maintenance of the vector. Suitable expression vectors include, e.g., plasmids, viral vectors, and the like. Large numbers of suitable vectors and promoters are known to those of skill in the art; many are commercially available for generating a subject recombinant construct. The following vectors are provided by way of example, and should not be construed in any way as limiting: Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus; poliovirus; adenovirus (see, e.g., Li et al., Invest. Opthalmol. Vis. Sci. (1994) 35: 2543-2549; Borras et al., Gene Ther. (1999) 6: 515-524; Li and Davidson, Proc. Natl. Acad. Sci. USA (1995) 92: 7700-7704; Sakamoto et al., H. Gene Ther. (1999) 5: 1088-1097; WO 94/12649, WO 93/03769; WO 93/19191; WO 94/28938; WO 95/11984 and WO 95/00655); adeno-associated virus (see, e.g., Ali et al., Hum. Gene Ther. (1998) 9: 81-86, Flannery et al., Proc. Natd. Acad. Sci. USA (1997) 94: 6916-6921; Bennett et al., Invest. Opthalmol. Vis. Sci. (1997) 38: 2857-2863; Jomary et al., Gene Ther. (1997) 4:683 690, Rolling et al., Hum. Gene Ther. (1999) 10: 641-648; Ali et al., Hum. Mol. Genet. (1996) 5: 591-594; Srivastava in WO 93/09239, Samulski et al., J. Vir. (1989) 63: 3822-3828; Mendelson et al., Virol. (1988) 166: 154-165; and Flotte et al., Proc. Natd. Acad. Sci. USA (1993) 90: 10613-10617); SV40; herpes simplex virus; human immunodeficiency virus (see, e.g., Miyoshi et al., Proc. Natl. Acad. Sci. USA (1997) 94: 10319-23; Takahashi et al., J. Virol. (1999) 73: 7812-7816); a retroviral vector (e.g., murine leukemia virus, spleen necrosis virus, and vectors derived from retroviruses such as Rous sarcoma virus, Harvey sarcoma virus, avian leukosis virus, human immunodeficiency virus, myeloproliferative sarcoma virus, and mammary tumor virus); and the like.

Additional expression vectors suitable for use are, e.g., without limitation, a lentivirus vector, a gamma retrovirus vector, a foamy virus vector, an adeno-associated virus vector, an adenovirus vector, a pox virus vector, a herpes virus vector, an engineered hybrid virus vector, a transposon mediated vector, and the like. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses.

In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

In some embodiments, an expression vector (e.g., a lentiviral vector) may be used to introduce the CAR into an immune cell or precursor thereof (e.g., a T cell). Accordingly, an expression vector (e.g., a lentiviral vector) of the present invention may comprise a nucleic acid encoding a CAR. In some embodiments, the expression vector (e.g., lentiviral vector) will comprise additional elements that will aid in the functional expression of the CAR encoded therein. In some embodiments, an expression vector comprising a nucleic acid encoding a CAR further comprises a mammalian promoter. In one embodiment, the vector further comprises an elongation-factor-1-alpha promoter (EF-1α promoter). Use of an EF-1α promoter may increase the efficiency in expression of downstream transgenes (e.g., a CAR encoding nucleic acid sequence). Physiologic promoters (e.g., an EF-1α promoter) may be less likely to induce integration mediated genotoxicity, and may abrogate the ability of the retroviral vector to transform stem cells Other physiological promoters suitable for use in a vector (e.g., a lentiviral vector) are known to those of skill in the art and may be incorporated into a vector of the present invention. In some embodiments, the vector (e.g., a lentiviral vector) further comprises a non-requisite cis acting sequence that may improve titers and gene expression. One non-limiting example of a non-requisite cis acting sequence is the central polypurine tract and central termination sequence (cPPT/CTS) which is important for efficient reverse transcription and nuclear import. Other non-requisite cis acting sequences are known to those of skill in the art and may be incorporated into a vector (e.g., lentiviral vector) of the present invention. In some embodiments, the vector further comprises a posttranscriptional regulatory element. Posttranscriptional regulatory elements may improve RNA translation, improve transgene expression and stabilize RNA transcripts. One example of a posttranscriptional regulatory element is the woodchuck hepatitis virus posttranscriptional regulatory element (WPRE). Accordingly, in some embodiments a vector for the present invention further comprises a WPRE sequence. Various posttranscriptional regulator elements are known to those of skill in the art and may be incorporated into a vector (e.g., a lentiviral vector) of the present invention. A vector of the present invention may further comprise additional elements such as a rev response element (RRE) for RNA transport, packaging sequences, and 5′ and 3′ long terminal repeats (LTRs). The term “long terminal repeat” or “LTR” refers to domains of base pairs located at the ends of retroviral DNAs which comprise U3, R and U5 regions. LTRs generally provide functions required for the expression of retroviral genes (e.g., promotion, initiation and polyadenylation of gene transcripts) and to viral replication. In one embodiment, a vector (e.g., lentiviral vector) of the present invention includes a 3′ U3 deleted LTR. Accordingly, a vector (e.g., lentiviral vector) of the present invention may comprise any combination of the elements described herein to enhance the efficiency of functional expression of transgenes. For example, a vector (e.g., lentiviral vector) of the present invention may comprise a WPRE sequence, cPPT sequence, RRE sequence, 5′LTR, 3′ U3 deleted LTR′ in addition to a nucleic acid encoding for a CAR.

Vectors of the present invention may be self-inactivating vectors. As used herein, the term “self-inactivating vector” refers to vectors in which the 3′ LTR enhancer promoter region (U3 region) has been modified (e.g., by deletion or substitution). A self-inactivating vector may prevent viral transcription beyond the first round of viral replication. Consequently, a self-inactivating vector may be capable of infecting and then integrating into a host genome (e.g., a mammalian genome) only once, and cannot be passed further. Accordingly, self-inactivating vectors may greatly reduce the risk of creating a replication-competent virus.

In some embodiments, a nucleic acid of the present invention may be RNA, e.g., in vitro synthesized RNA. Methods for in vitro synthesis of RNA are known to those of skill in the art; any known method can be used to synthesize RNA comprising a sequence encoding a CAR of the present disclosure. Methods for introducing RNA into a host cell are known in the art. See, e.g., Zhao et al. Cancer Res. (2010) 15: 9053. Introducing RNA comprising a nucleotide sequence encoding a CAR of the present disclosure into a host cell can be carried out in vitro or ex vivo or in vivo. For example, a host cell (e.g., an NK cell, a cytotoxic T lymphocyte, etc.) can be electroporated in vitro or ex vivo with RNA comprising a nucleotide sequence encoding a CAR of the present disclosure.

In order to assess the expression of a polypeptide or portions thereof, the expression vector to be introduced into a cell may also contain either a selectable marker gene or a reporter gene, or both, to facilitate identification and selection of expressing cells from the population of cells sought to be transfected or infected through viral vectors. In some embodiments, the selectable marker may be carried on a separate piece of DNA and used in a co-transfection procedure. Both selectable markers and reporter genes may be flanked with appropriate regulatory sequences to enable expression in the host cells. Useful selectable markers include, without limitation, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assessed at a suitable time after the DNA has been introduced into the recipient cells. Suitable reporter genes may include, without limitation, genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82).

### Methods of Generating Modified Immune Cells

The present invention provides methods for producing/generating a modified immune cell or precursor cell thereof (e.g., a T cell/NK cell/NKT cell). The cells are generally engineered by introducing a nucleic acid encoding a subject CAR (e.g., CD45 CAR). In some embodiments, the CAR is accompanied by a gene modification system that is capable of modifying the expression or sequence of the endogenous CD45 gene.

Methods of introducing nucleic acids into a cell include physical, biological and chemical methods. Physical methods for introducing a polynucleotide, such as RNA, into a host cell include calcium phosphate precipitation, lipofection, particle bombardment, microinjection, electroporation, and the like. RNA can be introduced into target cells using commercially available methods which include electroporation (Amaxa Nucleofector-II (Amaxa Biosystems, Cologne, Germany)), (ECM 830 (BTX) (Harvard Instruments, Boston, MA) or the Gene Pulser II (BioRad, Denver, CO), Multiporator (Eppendorf, Hamburg Germany). RNA can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12(8):861-70 (2001).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

In some embodiments, a nucleic acid encoding a subject CAR of the invention is introduced into a cell by an expression vector. Expression vectors comprising a nucleic acid encoding a subject CAR (e.g., CD45 CAR) are provided herein. Suitable expression vectors include lentivirus vectors, gamma retrovirus vectors, foamy virus vectors, adeno associated virus (AAV) vectors, adenovirus vectors, engineered hybrid viruses, naked DNA, including but not limited to transposon mediated vectors, such as Sleeping Beauty, Piggyback, and Integrases such as Phi31. Some other suitable expression vectors include herpes simplex virus (HSV) and retrovirus expression vectors.

Adenovirus expression vectors are based on adenoviruses, which have a low capacity for integration into genomic DNA but a high efficiency for transfecting host cells. Adenovirus expression vectors contain adenovirus sequences sufficient to: (a) support packaging of the expression vector and (b) to ultimately express the subject CAR in the host cell. In some embodiments, the adenovirus genome is a 36 kb, linear, double stranded DNA, where a foreign DNA sequence (e.g., a nucleic acid encoding a subject CAR) may be inserted to substitute large pieces of adenoviral DNA in order to make the expression vector of the present invention (see, e.g., Danthinne and Imperiale, Gene Therapy (2000) 7(20): 1707-1714).

Another expression vector is based on an adeno associated virus, which takes advantage of the adenovirus coupled systems. This AAV expression vector has a high frequency of integration into the host genome. It can infect non-dividing cells, thus making it useful for delivery of genes into mammalian cells, for example, in tissue cultures or in vivo. The AAV vector has a broad host range for infectivity. Details concerning the generation and use of AAV vectors are described in U.S. Pat. Nos. 5,139,941 and 4,797,368.

Retrovirus expression vectors are capable of integrating into the host genome, delivering a large amount of foreign genetic material, infecting a broad spectrum of species and cell types and being packaged in special cell lines. The retrovirus vector is constructed by inserting a nucleic acid (e.g., a nucleic acid encoding a subject CAR) into the viral genome at certain locations to produce a virus that is replication defective. Though the retrovirus vectors are able to infect a broad variety of cell types, integration and stable expression of the subject CAR, requires the division of host cells.

Lentivirus vectors are derived from lentiviruses, which are complex retroviruses that, in addition to the common retroviral genes gag, pol, and env, contain other genes with regulatory or structural function (see, e.g., U.S. Pat. Nos. 6,013,516 and 5,994,136). Some examples of lentiviruses include the human immunodeficiency viruses (HIV-1, HIV-2) and the simian immunodeficiency virus (SIV). Lentivirus vectors have been generated by multiply attenuating the HIV virulence genes, for example, the genes env, vif, vpr, vpu and nef are deleted making the vector biologically safe. Lentivirus vectors are capable of infecting non-dividing cells and can be used for both in vivo and ex vivo gene transfer and expression, e.g., of a nucleic acid encoding a subject CAR (see, e.g., U.S. Pat. No. 5,994,136).

Expression vectors including a nucleic acid of the present disclosure can be introduced into a host cell by any means known to persons skilled in the art. The expression vectors may include viral sequences for transfection, if desired. Alternatively, the expression vectors may be introduced by fusion, electroporation, biolistics, transfection, lipofection, or the like. The host cell may be grown and expanded in culture before introduction of the expression vectors, followed by the appropriate treatment for introduction and integration of the vectors. The host cells are then expanded and may be screened by virtue of a marker present in the vectors. Various markers that may be used are known in the art, and may include hprt, neomycin resistance, thymidine kinase, hygromycin resistance, etc. As used herein, the terms “cell,” “cell line,” and “cell culture” may be used interchangeably. In some embodiments, the host cell is an immune cell or precursor thereof, e.g., a T cell, an NK cell, or an NKT cell.

The present invention also provides genetically engineered cells which include and stably express a subject CAR of the present disclosure. In some embodiments, the genetically engineered cells are genetically engineered T-lymphocytes (T cells), regulatory T cells (Tregs), naive T cells (TN), memory T cells (for example, central memory T cells (TCM), effector memory cells (TEM)), natural killer cells (NK cells), natural killer T cells (NKT cells) and macrophages capable of giving rise to therapeutically relevant progeny. In one embodiment, the genetically engineered cells are autologous cells.

Modified cells (e.g., comprising a subject CAR) may be produced by stably transfecting host cells with an expression vector including a nucleic acid of the present disclosure. Additional methods to generate a modified cell of the present disclosure include, without limitation, chemical transformation methods (e.g., using calcium phosphate, dendrimers, liposomes and/or cationic polymers), non-chemical transformation methods (e.g., electroporation, optical transformation, gene electrotransfer and/or hydrodynamic delivery) and/or particle-based methods (e.g., impalefection, using a gene gun and/or magnetofection). Transfected cells expressing a subject CAR of the present disclosure may be expanded ex vivo.

Physical methods for introducing an expression vector into host cells include calcium phosphate precipitation, lipofection, particle bombardment, microinjection, electroporation, and the like. Methods for producing cells including vectors and/or exogenous nucleic acids are well-known in the art. See, e.g., Sambrook et al. (2001), Molecular Cloning: A Laboratory Manual, Cold Spring Harbor Laboratory, New York.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the nucleic acids in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Moreover, the nucleic acids may be introduced by any means, such as transducing the expanded T cells, transfecting the expanded T cells, and electroporating the expanded T cells. One nucleic acid may be introduced by one method and another nucleic acid may be introduced into the T cell by a different method.

### Gene Editing Systems

In some aspects, the disruption is carried out by gene editing using an RNA-guided nuclease such as a CRISPR-Cas system, such as CRISPR-Cas9 system, specific for the gene (e.g., CD45) being disrupted or modified in sequence. In some embodiments, an agent containing a Cas9 and a guide RNA (gRNA) containing a targeting domain, which targets a region of the genetic locus, is introduced into the cell. In some embodiments, the agent is or comprises a ribonucleoprotein (RNP) complex of a Cas9 polypeptide and a gRNA (Cas9/gRNA RNP). In some embodiments, the introduction includes contacting the agent or portion thereof with the cells in vitro, which can include cultivating or incubating the cell and agent for up to 24, 36 or 48 hours or 3, 4, 5, 6, 7, or 8 days. In some embodiments, the introduction further can include effecting delivery of the agent into the cells. In various embodiments, the methods, compositions and cells according to the present disclosure utilize direct delivery of ribonucleoprotein (RNP) complexes of Cas9 and gRNA to cells, for example by electroporation. In some embodiments, the RNP complexes include a gRNA that has been modified to include a 3′ poly-A tail and a 5′ Anti-Reverse Cap Analog (ARCA) cap.

The CRISPR/Cas9 system is a facile and efficient system for inducing targeted genetic alterations. Target recognition by the Cas9 protein requires a ‘seed’ sequence within the guide RNA (gRNA) and a conserved di-nucleotide containing protospacer adjacent motif (PAM) sequence upstream of the gRNA-binding region. The CRISPR/Cas9 system can thereby be engineered to cleave virtually any DNA sequence by redesigning the gRNA in cell lines (such as 293T cells), primary cells, and TCR T cells. The CRISPR/Cas9 system can simultaneously target multiple genomic loci by co-expressing a single Cas9 protein with two or more gRNAs, making this system suited for multiple gene editing or synergistic activation of target genes.

The Cas9 protein and guide RNA form a complex that identifies and cleaves target sequences. Cas9 is comprised of six domains: REC I, REC II, Bridge Helix, PAM interacting, HNH, and RuvC. The REC I domain binds the guide RNA, while the Bridge helix binds to target DNA. The HNH and RuvC domains are nuclease domains. Guide RNA is engineered to have a 5′ end that is complementary to the target DNA sequence. Upon binding of the guide RNA to the Cas9 protein, a conformational change occurs activating the protein. Once activated, Cas9 searches for target DNA by binding to sequences that match its protospacer adjacent motif (PAM) sequence. A PAM is a two or three nucleotide base sequence within one nucleotide downstream of the region complementary to the guide RNA. In one non-limiting example, the PAM sequence is 5′-NGG-3′. When the Cas9 protein finds its target sequence with the appropriate PAM, it melts the bases upstream of the PAM and pairs them with the complementary region on the guide RNA. Then the RuvC and HNH nuclease domains cut the target DNA after the third nucleotide base upstream of the PAM.

One non-limiting example of a CRISPR/Cas system used to inhibit gene expression, CRISPRi, is described in U.S. Patent Appl. Publ. No. US20140068797. CRISPRi induces permanent gene disruption that utilizes the RNA-guided Cas9 endonuclease to introduce DNA double stranded breaks which trigger error-prone repair pathways to result in frame shift mutations. A catalytically dead Cas9 lacks endonuclease activity. When coexpressed with a guide RNA, a DNA recognition complex is generated that specifically interferes with transcriptional elongation, RNA polymerase binding, or transcription factor binding. This CRISPRi system efficiently represses expression of targeted genes.

CRISPR/Cas gene disruption occurs when a guide nucleic acid sequence specific for a target gene and a Cas endonuclease are introduced into a cell and form a complex that enables the Cas endonuclease to introduce a double strand break at the target gene. In certain embodiments, the CRISPR/Cas system comprises an expression vector, such as, but not limited to, a pAd5F35-CRISPR vector. In other embodiments, the Cas expression vector induces expression of Cas9 endonuclease. Other endonucleases may also be used, including but not limited to, Casl2a (Cpf1), T7, Cas3, Cas8a, Cas8b, Cas10d, Cse1, Csy1, Csn2, Cas4, Cas10, Csm2, Cmr5, Fok1, other nucleases known in the art, and any combinations thereof.

In certain embodiments, inducing the Cas expression vector comprises exposing the cell to an agent that activates an inducible promoter in the Cas expression vector. In such embodiments, the Cas expression vector includes an inducible promoter, such as one that is inducible by exposure to an antibiotic (e.g., by tetracycline or a derivative of tetracycline, for example doxycycline). Other inducible promoters known by those of skill in the art can also be used. The inducing agent can be a selective condition (e.g., exposure to an agent, for example an antibiotic) that results in induction of the inducible promoter. This results in expression of the Cas expression vector.

As used herein, the term “guide RNA” or “gRNA” refer to any nucleic acid that promotes the specific association (or “targeting”) of an RNA-guided nuclease such as a Cas9 to a target sequence (e.g., a genomic or episomal sequence) in a cell.

As used herein, a “modular” or “dual RNA” guide comprises more than one, and typically two, separate RNA molecules, such as a CRISPR RNA (crRNA) and a trans-activating crRNA (tracrRNA), which are usually associated with one another, for example by duplexing. gRNAs and their component parts are described throughout the literature (see, e.g., Briner et al. Mol. Cell, 56(2), 333-339 (2014), which is incorporated by reference).

As used herein, a “unimolecular gRNA,” “chimeric gRNA,” or “single guide RNA (sgRNA)” comprises a single RNA molecule. The sgRNA may be a crRNA and tracrRNA linked together. For example, the 3′ end of the crRNA may be linked to the 5′ end of the tracrRNA. A crRNA and a tracrRNA may be joined into a single unimolecular or chimeric gRNA, for example, by means of a four nucleotide (e.g., GAAA) “tetraloop” or “linker” sequence bridging complementary regions of the crRNA (at its 3′ end) and the tracrRNA (at its 5′ end).

As used herein, a “repeat” sequence or region is a nucleotide sequence at or near the 3′ end of the crRNA which is complementary to an anti-repeat sequence of a tracrRNA.

As used herein, an “anti-repeat” sequence or region is a nucleotide sequence at or near the 5′ end of the tracrRNA which is complementary to the repeat sequence of a crRNA.

Additional details regarding guide RNA structure and function, including the gRNA/Cas9 complex for genome editing may be found in, at least, Mali et al. Science, 339(6121), 823-826 (2013); Jiang et al. Nat. Biotechnol. 31(3). 233-239 (2013); and Jinek et al. Science, 337(6096), 816-821 (2012); which are incorporated by reference herein.

As used herein, a “guide sequence” or “targeting sequence” refers to the nucleotide sequence of a gRNA, whether unimolecular or modular, that is fully or partially complementary to a target domain or target polynucleotide within a DNA sequence in the genome of a cell where editing is desired. Guide sequences are typically 10-30 nucleotides in length, preferably 16-24 nucleotides in length (for example, 16, 17, 18, 19, 20, 21, 22, 23 or 24 nucleotides in length), and are at or near the 5′ terminus of a Cas9 gRNA.

As used herein, a “target domain” or “target polynucleotide sequence” or “target sequence” is the DNA sequence in a genome of a cell that is complementary to the guide sequence of the gRNA.

In the context of formation of a CRISPR complex, “target sequence” refers to a sequence to which a guide sequence is designed to have some complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In certain embodiments, a target sequence is located in the nucleus or cytoplasm of a cell. In other embodiments, the target sequence may be within an organelle of a eukaryotic cell, for example, mitochondrion or nucleus. Typically, in the context of a CRISPR system, formation of a CRISPR complex (comprising a guide sequence hybridized to a target sequence and complexed with one or more Cas proteins) results in cleavage of one or both strands in or near (e.g., within about 1, 2, 3, 4, 5, 6, 7, 8, 9, 10, 20, 50 or more base pairs) the target sequence. As with the target sequence, it is believed that complete complementarity is not needed, provided this is sufficient to be functional.

In certain embodiments, one or more vectors driving expression of one or more elements of a CRISPR system are introduced into a host cell, such that expression of the elements of the CRISPR system direct formation of a CRISPR complex at one or more target sites. For example, a Cas nuclease, a crRNA, and a tracrRNA could each be operably linked to separate regulatory elements on separate vectors. Alternatively, two or more of the elements expressed from the same or different regulatory elements may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In certain embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g., each in a different intron, two or more in at least one intron, or all in a single intron).

In certain embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Additional domains that may form part of a fusion protein comprising a CRISPR enzyme are described in U.S. Patent Appl. Publ. No. US20110059502, incorporated herein by reference. In certain embodiments, a tagged CRISPR enzyme is used to identify the location of a target sequence.

Conventional viral and non-viral based gene transfer methods can be used to introduce nucleic acids in mammalian and non-mammalian cells or target tissues. Such methods can be used to administer nucleic acids encoding components of a CRISPR system to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell (Anderson, 1992, Science 256:808-813; and Yu, et al., 1994, Gene Therapy 1:13-26).

In some embodiments, the CRISPR/Cas is derived from a type II CRISPR/Cas system. In other embodiments, the CRISPR/Cas sytem is derived from a Cas9 nuclease. Exemplary Cas9 nucleases that may be used in the present invention include, but are not limited to, S. pyogenes Cas9 (SpCas9), S. aureus Cas9 (SaCas9), S. thermophilus Cas9 (StCas9), N. meningitidis Cas9 (NmCas9), C. jejuni Cas9 (CjCas9), and Geobacillus Cas9 (GeoCas9).

In general, Cas proteins comprise at least one RNA recognition and/or RNA binding domain. RNA recognition and/or RNA binding domains interact with the guiding RNA. Cas proteins can also comprise nuclease domains (i.e., DNase or RNase domains), DNA binding domains, helicase domains, RNAse domains, protein-protein interaction domains, dimerization domains, as well as other domains. The Cas proteins can be modified to increase nucleic acid binding affinity and/or specificity, alter an enzymatic activity, and/or change another property of the protein. In certain embodiments, the Cas-like protein of the fusion protein can be derived from a wild type Cas9 protein or fragment thereof. In other embodiments, the Cas can be derived from modified Cas9 protein. For example, the amino acid sequence of the Cas9 protein can be modified to alter one or more properties (e.g., nuclease activity, affinity, stability, and so forth) of the protein. Alternatively, domains of the Cas9 protein not involved in RNA-guided cleavage can be eliminated from the protein such that the modified Cas9 protein is smaller than the wild type Cas9 protein. In general, a Cas9 protein comprises at least two nuclease (i.e., DNase) domains. For example, a Cas9 protein can comprise a RuvC-like nuclease domain and a HNH-like nuclease domain. The RuvC and HNH domains work together to cut single strands to make a double-stranded break in DNA. (Jinek, et al., 2012, Science, 337:816-821). In certain embodiments, the Cas9-derived protein can be modified to contain only one functional nuclease domain (either a RuvC-like or a HNH-like nuclease domain). For example, the Cas9-derived protein can be modified such that one of the nuclease domains is deleted or mutated such that it is no longer functional (i.e., the nuclease activity is absent). In some embodiments in which one of the nuclease domains is inactive, the Cas9-derived protein is able to introduce a nick into a double-stranded nucleic acid (such protein is termed a “nickase”), but not cleave the double-stranded DNA. In any of the above-described embodiments, any or all of the nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art.

In one non-limiting embodiment, a vector drives the expression of the CRISPR system. The art is replete with suitable vectors that are useful in the present invention. The vectors to be used are suitable for replication and, optionally, integration in eukaryotic cells. Typical vectors contain transcription and translation terminators, initiation sequences, and promoters useful for regulation of the expression of the desired nucleic acid sequence. The vectors of the present invention may also be used for nucleic acid standard gene delivery protocols. Methods for gene delivery are known in the art (U.S. Pat. Nos. 5,399,346, 5,580,859 & 5,589,466, incorporated by reference herein in their entireties).

Further, the vector may be provided to a cell in the form of a viral vector. Viral vector technology is well known in the art and is described, for example, in Sambrook et al. (4th Edition, Molecular Cloning: A Laboratory Manual, Cold Spring Harbor Laboratory, New York, 2012), and in other virology and molecular biology manuals. Viruses, which are useful as vectors include, but are not limited to, retroviruses, adenoviruses, adeno-associated viruses, herpes viruses, Sindbis virus, gammaretrovirus and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

In some embodiments, guide RNA(s) and Cas9 can be delivered to a cell as a ribonucleoprotein (RNP) complex (e.g., a Cas9/RNA-protein complex). RNPs are comprised of purified Cas9 protein complexed with gRNA and are well known in the art to be efficiently delivered to multiple types of cells, including but not limited to stem cells and immune cells (Addgene, Cambridge, MA, Mirus Bio LLC, Madison, WI). In some embodiments, the Cas9/RNA-protein complex is delivered into a cell by electroporation.

In some embodiments, a gene edited modified cell of the present disclosure is edited using CRISPR/Cas9 to disrupt or modify an endogenous gene locus encoding CD45

Suitable gRNAs for use in disrupting or modifying CD45 are set forth in Tables 3-5 (SEQ ID NOs: 212-252). It will be understood to those of skill in the art that guide RNA sequences may be recited with a thymidine (T) or a uridine (U) nucleotide.

In certain embodiments, the gene editing system comprises a base editor CRISPR systems capable of making several modifications to the endogenous CD45 gene locus simultaneously. Suitable nucleic acids useful for use in such a gene editor system are set forth in SEQ ID NOs: 253-258.

Accordingly, provided in the invention is a modified immune cell or precursor cell thereof comprising a CRISPR-mediated modification in an endogenous gene locus encoding CD45, wherein the modification is capable of downregulating gene expression or modifying the sequence of endogenous CD45; and an exogenous T cell receptor (TCR) and/or chimeric antigen receptor (CAR) comprising affinity for an antigen on a target cell.

Non-limiting types of CRISPR-mediated modifications include a substitution, an insertion, a deletion, and an insertion/deletion (INDEL). The modification can be located in any part of the endogenous gene locus encoding CD45, including but not limited to an exon, a splice donor, or a splice acceptor.

In certain embodiments, the guide RNA comprises the nucleic acid sequence set forth in any one of SEQ ID NOs: 212-252.

TABLE 1
PTPRC/CD45 Sequences

| SEQ | | |
|---|---|---|
| ID | | |
| NO: | Description | Sequence |
| 1 | PTPRC full | GACATCATCACCTAGCAGTTCATGCAGCTAGCAAGTGGTTTGT |
| | length | TCTTAGGGTAACAGAGGAGGAAATTGTTCCTCGTCTGATAAG |
| | (CD45RABC) | ACAACAGTGGAGAAAGGACGCATGCTGTTTCTTAGGGACACG |
| | CDNA | GCTGACTTCCAGATATGACCATGTATTTGTGGCTTAAACTCTT |
| | (NM_002838.5): | GGCATTTGGCTTTGCCTTTCTGGACACAGAAGTATTTGTGACA |
| | | GGGCAAAGCCCAACACCTTCCCCCACTGGATTGACTACAGCA |
| | | AAGATGCCCAGTGTTCCACTTTCAAGTGACCCCTTACCTACTC |
| | | ACACCACTGCATTCTCACCCGCAAGCACCTTTGAAAGAGAAA |
| | | ATGACTTCTCAGAGACCACAACTTCTCTTAGTCCAGACAATAC |
| | | TTCCACCCAAGTATCCCCGGACTCTTTGGATAATGCTAGTGCT |
| | | TTTAATACCACAGGTGTTTCATCAGTACAGACGCCTCACCTTC |
| | | CCACGCACGCAGACTCGCAGACGCCCTCTGCTGGAACTGACA |
| | | CGCAGACATTCAGCGGCTCCGCCGCCAATGCAAAACTCAACC |
| | | CTACCCCAGGCAGCAATGCTATCTCAGATGTCCCAGGAGAGA |
| | | GGAGTACAGCCAGCACCTTTCCTACAGACCCAGTTTCCCCATT |
| | | GACAACCACCCTCAGCCTTGCACACCACAGCTCTGCTGCCTTA |
| | | CCTGCACGCACCTCCAACACCACCATCACAGCGAACACCTCA |
| | | GATGCCTACCTTAATGCCTCTGAAACAACCACTCTGAGCCCTT |
| | | CTGGAAGCGCTGTCATTTCAACCACAACAATAGCTACTACTCC |
| | | ATCTAAGCCAACATGTGATGAAAAATATGCAAACATCACTGT |
| | | GGATTACTTATATAACAAGGAAACTAAATTATTTACAGCAAA |
| | | GCTAAATGTTAATGAGAATGTGGAATGTGGAAACAATACTTG |
| | | CACAAACAATGAGGTGCATAACCTTACAGAATGTAAAAATGC |
| | | GTCTGTTTCCATATCTCATAATTCATGTACTGCTCCTGATAAG |
| | | ACATTAATATTAGATGTGCCACCAGGGGTTGAAAAGTTTCAGT |
| | | TACATGATTGTACACAAGTTGAAAAAGCAGATACTACTATTTG |
| | | TTTAAAATGGAAAAATATTGAAACCTTTACTTGTGATACACAG |
| | | AATATTACCTACAGATTTCAGTGTGGTAATATGATATTTGATA |
| | | ATAAAGAAATTAAATTAGAAAACCTTGAACCCGAACATGAGT |
| | | ATAAGTGTGACTCAGAAATACTCTATAATAACCACAAGTTTAC |
| | | TAACGCAAGTAAAATTATTAAAACAGATTTTGGGAGTCCAGG |
| | | AGAGCCTCAGATTATTTTTTGTAGAAGTGAAGCTGCACATCAA |
| | | GGAGTAATTACCTGGAATCCCCCTCAAAGATCATTTCATAATT |
| | | TTACCCTCTGTTATATAAAAGAGACAGAAAAAGATTGCCTCA |
| | | ATCTGGATAAAAACCTGATCAAATATGATTTGCAAAATTTAA |
| | | AACCTTATACGAAATATGTTTTATCATTACATGCCTACATCAT |
| | | TGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTACATAAG |
| | | AAAAGATCCTGCAATTTAGATGAACAGCAGGAGCTTGTTGAA |
| | | AGGGATGATGAAAAACAACTGATGAATGTGGAGCCAATCCAT |
| | | GCAGATATTTTGTTGGAAACTTATAAGAGGAAGATTGCTGAT |
| | | GAAGGAAGACTTTTTCTGGCTGAATTTCAGAGCATCCCGCGG |
| | | GTGTTCAGCAAGTTTCCTATAAAGGAAGCTCGAAAGCCCTTTA |
| | | ACCAGAATAAAAACCGTTATGTTGACATTCTTCCTTATGATTA |
| | | TAACCGTGTTGAACTCTCTGAGATAAACGGAGATGCAGGGTC |
| | | AAACTACATAAATGCCAGCTATATTGATGGTTTCAAAGAACC |
| | | CAGGAAATACATTGCTGCACAAGGTCCCAGGGATGAAACTGT |
| | | TGATGATTTCTGGAGGATGATTTGGGAACAGAAAGCCACAGT |
| | | TATTGTCATGGTCACTCGATGTGAAGAAGGAAACAGGAACAA |
| | | GTGTGCAGAATACTGGCCGTCAATGGAAGAGGGCACTCGGGC |
| | | TTTTGGAGATGTTGTTGTAAAGATCAACCAGCACAAAAGATG |
| | | TCCAGATTACATCATTCAGAAATTGAACATTGTAAATAAAAA |
| | | AGAAAAAGCAACTGGAAGAGAGGTGACTCACATTCAGTTCAC |
| | | CAGCTGGCCAGACCACGGGGTGCCTGAGGATCCTCACTTGCT |
| | | CCTCAAACTGAGAAGGAGAGTGAATGCCTTCAGCAATTTCTTC |
| | | AGTGGTCCCATTGTGGTGCACTGCAGTGCTGGTGTTGGGCGCA |
| | | CAGGAACCTATATCGGAATTGATGCCATGCTAGAAGGCCTGG |
| | | AAGCCGAGAACAAAGTGGATGTTTATGGTTATGTTGTCAAGC |
| | | TAAGGCGACAGAGATGCCTGATGGTTCAAGTAGAGGCCCAGT |
| | | ACATCTTGATCCATCAGGCTTTGGTGGAATACAATCAGTTTGG |
| | | AGAAACAGAAGTGAATTTGTCTGAATTACATCCATATCTACAT |
| | | AACATGAAGAAAAGGGATCCACCCAGTGAGCCGTCTCCACTA |
| | | GAGGCTGAATTCCAGAGACTTCCTTCATATAGGAGCTGGAGG |
| | | ACACAGCACATTGGAAATCAAGAAGAAAATAAAAGTAAAAA |
| | | CAGGAATTCTAATGTCATCCCATATGACTATAACAGAGTGCCA |
| | | CTTAAACATGAGCTGGAAATGAGTAAAGAGAGTGAGCATGAT |
| | | TCAGATGAATCCTCTGATGATGACAGTGATTCAGAGGAACCA |
| | | AGCAAATACATCAATGCATCTTTTATAATGAGCTACTGGAAAC |
| | | CTGAAGTGATGATTGCTGCTCAGGGACCACTGAAGGAGACCA |
| | | TTGGTGACTTTTGGCAGATGATCTTCCAAAGAAAAGTCAAAGT |
| | | TATTGTTATGCTGACAGAACTGAAACATGGAGACCAGGAAAT |
| | | CTGTGCTCAGTACTGGGGAGAAGGAAAGCAAACATATGGAGA |
| | | TATTGAAGTTGACCTGAAAGACACAGACAAATCTTCAACTTAT |
| | | ACCCTTCGTGTCTTTGAACTGAGACATTCCAAGAGGAAAGACT |
| | | CTCGAACTGTGTACCAGTACCAATATACAAACTGGAGTGTGG |
| | | AGCAGCTTCCTGCAGAACCCAAGGAATTAATCTCTATGATTCA |
| | | GGTCGTCAAACAAAAACTTCCCCAGAAGAATTCCTCTGAAGG |
| | | GAACAAGCATCACAAGAGTACACCTCTACTCATTCACTGCAG |
| | | GGATGGATCTCAGCAAACGGGAATATTTTGTGCTTTGTTAAAT |
| | | CTCTTAGAAAGTGCGGAAACAGAAGAGGTAGTGGATATTTTT |
| | | CAAGTGGTAAAAGCTCTACGCAAAGCTAGGCCAGGCATGGTT |
| | | TCCACATTCGAGCAATATCAATTCCTATATGACGTCATTGCCA |
| | | GCACCTACCCTGCTCAGAATGGACAAGTAAAGAAAAACAACC |
| | | ATCAAGAAGATAAAATTGAATTTGATAATGAAGTGGACAAAG |
| | | TAAAGCAGGATGCTAATTGTGTTAATCCACTTGGTGCCCCAGA |
| | | AAAGCTCCCTGAAGCAAAGGAACAGGCTGAAGGTTCTGAACC |
| | | CACGAGTGGCACTGAGGGGCCAGAACATTCTGTCAATGGTCC |
| | | TGCAAGTCCAGCTTTAAATCAAGGTTCATAGGAAAAGACATA |
| | | AATGAGGAAACTCCAAACCTCCTGTTAGCTGTTATTTCTATTT |
| | | TTGTAGAAGTAGGAAGTGAAAATAGGTATACAGTGGATTAAT |
| | | TAAATGCAGCGAACCAATATTTGTAGAAGGGTTATATTTTACT |
| | | ACTGTGGAAAAATATTTAAGATAGTTTTGCCAGAACAGTTTGT |
| | | ACAGACGTATGCTTATTTTAAAATTTTATCTCTTATTCAGTAA |
| | | AAAACAACTTCTTTGTAATCGTTATGTGTGTATATGTATGTGT |
| | | GTATGGGTGTGTGTTTGTGTGAGAGACAGAGAAAGAGAGAGA |
| | | ATTCTTTCAAGTGAATCTAAAAGCTTTTGCTTTTCCTTTGTTTT |
| | | TATGAAGAAAAAATACATTTTATATTAGAAGTGTTAACTTAGC |
| | | TTGAAGGATCTGTTTTTAAAAATCATAAACTGTGTGCAGACTC |
| | | AATAAAATCATGTACATTTCTGAAATGACCTCAAGATGTCCTC |
| | | CTTGTTCTACTCATATATATCTATCTTATATAGTTTACTATTTT |
| | | ACTTCTAGAGATAGTACATAAAGGTGGTATGTGTGTGTATGCT |
| | | ACTACAAAAAAGTTGTTAACTAAATTAACATTGGGAAATCTT |
| | | ATATTCCATATATTAGCATTTAGTCCAATGTCTTTTTAAGCTTA |
| | | TTTAATTAAAAAATTTCCAGTGAGCTTATCATGCTGTCTTTAC |
| | | ATGGGGTTTTCAATTTTGCATGCTCGATTATTCCCTGTACAAT |
| | | ATTTAAAATTTATTGCTTGATACTTTTGACAACAAATTAGGTT |
| | | TTGTACAATTGAACTTAAATAAATGTCATTAAAATAAATAAAT |
| | | GCAATATGTATTAATATTCATTGTATAAAAATAGAAGAATAC |
| | | AAACATATTTGTTAAATATTTACATATGAAATTTAATATAGCT |
| | | ATTTTTATGGAATTTTTCATTGATATGAAAAATATGATATTGC |
| | | ATATGCATAGTTCCCATGTTAAATCCCATTCATAACTTTCATT |
| | | AAAGCATTTACTTTGAATTTCTCCAATGCTTAGAATGTTTTTAC |
| | | CAGGAATGGATGTCGCTAATCATAATAAAATTCAACCATTATT |
| | | TTTTTCTTGTTTATAATACATTGTGTTATATGTTCAAATATGAA |
| | | ATGTGTATGCACCTATTGAAATATGTTTAATGCATTTATTAAC |
| | | ATTTGCAGGACACTTTTACAGGCCCCAATTATCCAATAGTCTA |
| | | ATAATTGTTTAAGATCTAGAAAAAAAAAATCAAGAATAGTGG |
| | | TATTTTTCATGAAGTAATAAAAACTCGTTTTGGTGAA |
| 2 | PTPRC full | DIIT*QFMQLASGLFLG*QRRKLFLV**DNSGERTHAVS*GHG*LP |
| | length | DMTMYLWLKLLAFGFAFLDTEVFVTGQSPTPSPTGLTTAKMPSV |
| | (CD45RABC) | PLSSDPLPTHTTAFSPASTFERENDFSETTTSLSPDNTSTQVSPDSL |
| | AA | DNASAFNTTGVSSVQTPHLPTHADSQTPSAGTDTQTFSGSAANA |
| | (NM_002838.5): | KLNPTPGSNAISDVPGERSTASTFPTDPVSPLTTTLSLAHHSSAAL |
| | | PARTSNTTITANTSDAYLNASETTTLSPSGSAVISTTTIATTPSKPT |
| | | CDEKYANITVDYLYNKETKLFTAKLNVNENVECGNNTCTNNEV |
| | | HNLTECKNASVSISHNSCTAPDKTLILDVPPGVEKFQLHDCTQVE |
| | | KADTTICLKWKNIETFTCDTQNITYRFQCGNMIFDNKEIKLENLE |
| | | PEHEYKCDSEILYNNHKFTNASKIIKTDFGSPGEPQIIFCRSEAAHQ |
| | | GVITWNPPQRSFHNFTLCYIKETEKDCLNLDKNLIKYDLQNLKPY |
| | | TKYVLSLHAYIIAKVQRNGSAAMCHFTTKSAPPSQVWNMTVSM |
| | | TSDNSMHVKCRPPRDRNGPHERYHLEVEAGNTLVRNESHKNCD |
| | | FRVKDLQYSTDYTFKAYFHNGDYPGEPFILHHSTSYNSKALIAFL |
| | | AFLIIVTSIALLVVLYKIYDLHKKRSCNLDEQQELVERDDEKQLM |
| | | NVEPIHADILLETYKRKIADEGRLFLAEFQSIPRVFSKFPIKEARKP |
| | | FNQNKNRYVDILPYDYNRVELSEINGDAGSNYINASYIDGFKEPR |
| | | KYIAAQGPRDETVDDFWRMIWEQKATVIVMVTRCEEGNRNKCA |
| | | EYWPSMEEGTRAFGDVVVKINQHKRCPDYIIQKLNIVNKKEKAT |
| | | GREVTHIQFTSWPDHGVPEDPHLLLKLRRRVNAFSNFFSGPIVVH |
| | | CSAGVGRTGTYIGIDAMLEGLEAENKVDVYGYVVKLRRQRCLM |
| | | VQVEAQYILIHQALVEYNQFGETEVNLSELHPYLHNMKKRDPPS |
| | | EPSPLEAEFQRLPSYRSWRTQHIGNQEENKSKNRNSNVIPYDYNR |
| | | VPLKHELEMSKESEHDSDESSDDDSDSEEPSKYINASFIMSYWKP |
| | | EVMIAAQGPLKETIGDFWQMIFQRKVKVIVMLTELKHGDQEICA |
| | | QYWGEGKQTYGDIEVDLKDTDKSSTYTLRVFELRHSKRKDSRT |
| | | VYQYQYTNWSVEQLPAEPKELISMIQVVKQKLPQKNSSEGNKH |
| | | HKSTPLLIHCRDGSQQTGIFCALLNLLESAETEEVVDIFQVVKALR |
| | | KARPGMVSTFEQYQFLYDVIASTYPAQNGQVKKNNHQEDKIEFD |
| | | NEVDKVKQDANCVNPLGAPEKLPEAKEQAEGSEPTSGTEGPEHS |
| | | VNGPASPALNQGS*EKT*MRKLQTSC*LLFLFL*K*EVKIGIQWIN |
| | | *MQRTNICRRVIFYYCGKIFKIVLPEQFVQTYAYFKILSLIQ*KTTS |
| | | L*SLCVYMYVCMGVCLCERQRKRENSFK*I*KLLLFLCFYEEKIH |
| | | FILEVLT*LEGSVFKNHKLCADSIKSCTFLK*PQDVLLVLLIYIYLI* |
| | | FTILLLEIVHKGGMCVYATTKKLLTKLTLGNLIFHILAFSPMSF*A |
| | | YLIKKFPVSLSCCLYMGFSILHARLFPVQYLKFIA*YF*QQIRFCTI |
| | | ELK*MSLK*INAICINIHCIKIEEYKHIC*IFTYEI*YSYFYGIFH*YE |
| | | KYDIAYA*FPC*IPFITFIKAFTLNFSNA*NVFTRNGCR*S**NSTIIF |
| | | FLFIIHCVICSNMKCVCTY*NMFNAFINICRTLLQAPIIQ*SNNCLR |
| | | SRKKKSRIVVFFMK**KLVLV |
| 3 | PTPRC without | GACATCATCACCTAGCAGTTCATGCAGCTAGCAAGTGGT |
| | exon 4, 5, 6 | TTGTTCTTAGGGTAACAGAGGAGGAAATTGTTCCTCGTC |
| | (CD45RO) | TGATAAGACAACAGTGGAGAAAGGACGCATGCTGTTTCT |
| | CDNA: | TAGGGACACGGCTGACTTCCAGATATGACCATGTATTTG |
| | | TGGCTTAAACTCTTGGCATTTGGCTTTGCCTTTCTGGACA |
| | | CAGAAGTATTTGTGACAGGGCAAAGCCCAACACCTTCCC |
| | | CCACTGATGCCTACCTTAATGCCTCTGAAACAACCACTC |
| | | TGAGCCCTTCTGGAAGCGCTGTCATTTCAACCACAACAA |
| | | TAGCTACTACTCCATCTAAGCCAACATGTGATGAAAAAT |
| | | ATGCAAACATCACTGTGGATTACTTATATAACAAGGAAA |
| | | CTAAATTATTTACAGCAAAGCTAAATGTTAATGAGAATG |
| | | TGGAATGTGGAAACAATACTTGCACAAACAATGAGGTG |
| | | CATAACCTTACAGAATGTAAAAATGCGTCTGTTTCCATA |
| | | TCTCATAATTCATGTACTGCTCCTGATAAGACATTAATAT |
| | | TAGATGTGCCACCAGGGGTTGAAAAGTTTCAGTTACATG |
| | | ATTGTACACAAGTTGAAAAAGCAGATACTACTATTTGTT |
| | | TAAAATGGAAAAATATTGAAACCTTTACTTGTGATACAC |
| | | AGAATATTACCTACAGATTTCAGTGTGGTAATATGATAT |
| | | TTGATAATAAAGAAATTAAATTAGAAAACCTTGAACCCG |
| | | AACATGAGTATAAGTGTGACTCAGAAATACTCTATAATA |
| | | ACCACAAGTTTACTAACGCAAGTAAAATTATTAAAACAG |
| | | ATTTTGGGAGTCCAGGAGAGCCTCAGATTATTTTTTGTA |
| | | GAAGTGAAGCTGCACATCAAGGAGTAATTACCTGGAAT |
| | | CCCCCTCAAAGATCATTTCATAATTTTACCCTCTGTTATA |
| | | TAAAAGAGACAGAAAAAGATTGCCTCAATCTGGATAAA |
| | | AACCTGATCAAATATGATTTGCAAAATTTAAAACCTTAT |
| | | ACGAAATATGTTTTATCATTACATGCCTACATCATTGCA |
| | | AAAGTGCAACGTAATGGAAGTGCTGCAATGTGTCATTTC |
| | | ACAACTAAAAGTGCTCCTCCAAGCCAGGTCTGGAACATG |
| | | ACTGTCTCCATGACATCAGATAATAGTATGCATGTCAAG |
| | | TGTAGGCCTCCCAGGGACCGTAATGGCCCCCATGAACGT |
| | | TACCATTTGGAAGTTGAAGCTGGAAATACTCTGGTTAGA |
| | | AATGAGTCGCATAAGAATTGCGATTTCCGTGTAAAAGAT |
| | | CTTCAATATTCAACAGACTACACTTTTAAGGCCTATTTTC |
| | | ACAATGGAGACTATCCTGGAGAACCCTTTATTTTACATC |
| | | ATTCAACATCTTATAATTCTAAGGCACTGATAGCATTTCT |
| | | GGCATTTCTGATTATTGTGACATCAATAGCCCTGCTTGTT |
| | | GTTCTCTACAAAATCTATGATCTACATAAGAAAAGATCC |
| | | TGCAATTTAGATGAACAGCAGGAGCTTGTTGAAAGGGAT |
| | | GATGAAAAACAACTGATGAATGTGGAGCCAATCCATGC |
| | | AGATATTTTGTTGGAAACTTATAAGAGGAAGATTGCTGA |
| | | TGAAGGAAGACTTTTTCTGGCTGAATTTCAGAGCATCCC |
| | | GCGGGTGTTCAGCAAGTTTCCTATAAAGGAAGCTCGAAA |
| | | GCCCTTTAACCAGAATAAAAACCGTTATGTTGACATTCT |
| | | TCCTTATGATTATAACCGTGTTGAACTCTCTGAGATAAA |
| | | CGGAGATGCAGGGTCAAACTACATAAATGCCAGCTATAT |
| | | TGATGGTTTCAAAGAACCCAGGAAATACATTGCTGCACA |
| | | AGGTCCCAGGGATGAAACTGTTGATGATTTCTGGAGGAT |
| | | GATTTGGGAACAGAAAGCCACAGTTATTGTCATGGTCAC |
| | | TCGATGTGAAGAAGGAAACAGGAACAAGTGTGCAGAAT |
| | | ACTGGCCGTCAATGGAAGAGGGCACTCGGGCTTTTGGAG |
| | | ATGTTGTTGTAAAGATCAACCAGCACAAAAGATGTCCAG |
| | | ATTACATCATTCAGAAATTGAACATTGTAAATAAAAAAG |
| | | AAAAAGCAACTGGAAGAGAGGTGACTCACATTCAGTTC |
| | | ACCAGCTGGCCAGACCACGGGGTGCCTGAGGATCCTCAC |
| | | TTGCTCCTCAAACTGAGAAGGAGAGTGAATGCCTTCAGC |
| | | AATTTCTTCAGTGGTCCCATTGTGGTGCACTGCAGTGCTG |
| | | GTGTTGGGCGCACAGGAACCTATATCGGAATTGATGCCA |
| | | TGCTAGAAGGCCTGGAAGCCGAGAACAAAGTGGATGTT |
| | | TATGGTTATGTTGTCAAGCTAAGGCGACAGAGATGCCTG |
| | | ATGGTTCAAGTAGAGGCCCAGTACATCTTGATCCATCAG |
| | | GCTTTGGTGGAATACAATCAGTTTGGAGAAACAGAAGTG |
| | | AATTTGTCTGAATTACATCCATATCTACATAACATGAAG |
| | | AAAAGGGATCCACCCAGTGAGCCGTCTCCACTAGAGGCT |
| | | GAATTCCAGAGACTTCCTTCATATAGGAGCTGGAGGACA |
| | | CAGCACATTGGAAATCAAGAAGAAAATAAAAGTAAAAA |
| | | CAGGAATTCTAATGTCATCCCATATGACTATAACAGAGT |
| | | GCCACTTAAACATGAGCTGGAAATGAGTAAAGAGAGTG |
| | | AGCATGATTCAGATGAATCCTCTGATGATGACAGTGATT |
| | | CAGAGGAACCAAGCAAATACATCAATGCATCTTTTATAA |
| | | TGAGCTACTGGAAACCTGAAGTGATGATTGCTGCTCAGG |
| | | GACCACTGAAGGAGACCATTGGTGACTTTTGGCAGATGA |
| | | TCTTCCAAAGAAAAGTCAAAGTTATTGTTATGCTGACAG |
| | | AACTGAAACATGGAGACCAGGAAATCTGTGCTCAGTACT |
| | | GGGGAGAAGGAAAGCAAACATATGGAGATATTGAAGTT |
| | | GACCTGAAAGACACAGACAAATCTTCAACTTATACCCTT |
| | | CGTGTCTTTGAACTGAGACATTCCAAGAGGAAAGACTCT |
| | | CGAACTGTGTACCAGTACCAATATACAAACTGGAGTGTG |
| | | GAGCAGCTTCCTGCAGAACCCAAGGAATTAATCTCTATG |
| | | ATTCAGGTCGTCAAACAAAAACTTCCCCAGAAGAATTCC |
| | | TCTGAAGGGAACAAGCATCACAAGAGTACACCTCTACTC |
| | | ATTCACTGCAGGGATGGATCTCAGCAAACGGGAATATTT |
| | | TGTGCTTTGTTAAATCTCTTAGAAAGTGCGGAAACAGAA |
| | | GAGGTAGTGGATATTTTTCAAGTGGTAAAAGCTCTACGC |
| | | AAAGCTAGGCCAGGCATGGTTTCCACATTCGAGCAATAT |
| | | CAATTCCTATATGACGTCATTGCCAGCACCTACCCTGCTC |
| | | AGAATGGACAAGTAAAGAAAAACAACCATCAAGAAGAT |
| | | AAAATTGAATTTGATAATGAAGTGGACAAAGTAAAGCA |
| | | GGATGCTAATTGTGTTAATCCACTTGGTGCCCCAGAAAA |
| | | GCTCCCTGAAGCAAAGGAACAGGCTGAAGGTTCTGAAC |
| | | CCACGAGTGGCACTGAGGGGCCAGAACATTCTGTCAATG |
| | | GTCCTGCAAGTCCAGCTTTAAATCAAGGTTCATAGGAAA |
| | | AGACATAAATGAGGAAACTCCAAACCTCCTGTTAGCTGT |
| | | TATTTCTATTTTTGTAGAAGTAGGAAGTGAAAATAGGTA |
| | | TACAGTGGATTAATTAAATGCAGCGAACCAATATTTGTA |
| | | GAAGGGTTATATTTTACTACTGTGGAAAAATATTTAAGA |
| | | TAGTTTTGCCAGAACAGTTTGTACAGACGTATGCTTATTT |
| | | TAAAATTTTATCTCTTATTCAGTAAAAAACAACTTCTTTG |
| | | TAATCGTTATGTGTGTATATGTATGTGTGTATGGGTGTGT |
| | | GTTTGTGTGAGAGACAGAGAAAGAGAGAGAATTCTTTC |
| | | AAGTGAATCTAAAAGCTTTTGCTTTTCCTTTGTTTTTATG |
| | | AAGAAAAAATACATTTTATATTAGAAGTGTTAACTTAGC |
| | | TTGAAGGATCTGTTTTTAAAAATCATAAACTGTGTGCAG |
| | | ACTCAATAAAATCATGTACATTTCTGAAATGACCTCAAG |
| | | ATGTCCTCCTTGTTCTACTCATATATATCTATCTTATATA |
| | | GTTTACTATTTTACTTCTAGAGATAGTACATAAAGGTGG |
| | | TATGTGTGTGTATGCTACTACAAAAAAGTTGTTAACTAA |
| | | ATTAACATTGGGAAATCTTATATTCCATATATTAGCATTT |
| | | AGTCCAATGTCTTTTTAAGCTTATTTAATTAAAAAATTTC |
| | | CAGTGAGCTTATCATGCTGTCTTTACATGGGGTTTTCAAT |
| | | TTTGCATGCTCGATTATTCCCTGTACAATATTTAAAATTT |
| | | ATTGCTTGATACTTTTGACAACAAATTAGGTTTTGTACAA |
| | | TTGAACTTAAATAAATGTCATTAAAATAAATAAATGCAA |
| | | TATGTATTAATATTCATTGTATAAAAATAGAAGAATACA |
| | | AACATATTTGTTAAATATTTACATATGAAATTTAATATA |
| | | GCTATTTTTATGGAATTTTTCATTGATATGAAAAATATGA |
| | | TATTGCATATGCATAGTTCCCATGTTAAATCCCATTCATA |
| | | ACTTTCATTAAAGCATTTACTTTGAATTTCTCCAATGCTT |
| | | AGAATGTTTTTACCAGGAATGGATGTCGCTAATCATAAT |
| | | AAAATTCAACCATTATTTTTTTCTTGTTTATAATACATTG |
| | | TGTTATATGTTCAAATATGAAATGTGTATGCACCTATTG |
| | | AAATATGTTTAATGCATTTATTAACATTTGCAGGACACTT |
| | | TTACAGGCCCCAATTATCCAATAGTCTAATAATTGTTTA |
| | | AGATCTAGAAAAAAAAAATCAAGAATAGTGGTATTTTTC |
| | | ATGAAGTAATAAAAACTCGTTTTGGTGAA |
| 4 | PTPRC without | DIIT*QFMQLASGLFLG*QRRKLFLV**DNSGERTHAVS*GH |
| | exon 4, 5, 6 | G*LPDMTMYLWLKLLAFGFAFLDTEVFVTGQSPTPSPTDA |
| | (CD45RO) AA: | YLNASETTTLSPSGSAVISTTTIATTPSKPTCDEKYANITVDY |
| | | LYNKETKLFTAKLNVNENVECGNNTCTNNEVHNLTECKN |
| | | ASVSISHNSCTAPDKTLILDVPPGVEKFQLHDCTQVEKADT |
| | | TICLKWKNIETFTCDTQNITYRFQCGNMIFDNKEIKLENLEP |
| | | EHEYKCDSEILYNNHKFTNASKIIKTDFGSPGEPQIIFCRSEA |
| | | AHQGVITWNPPQRSFHNFTLCYIKETEKDCLNLDKNLIKYD |
| | | LQNLKPYTKYVLSLHAYIIAKVQRNGSAAMCHFTTKSAPPS |
| | | QVWNMTVSMTSDNSMHVKCRPPRDRNGPHERYHLEVEA |
| | | GNTLVRNESHKNCDFRVKDLQYSTDYTFKAYFHNGDYPG |
| | | EPFILHHSTSYNSKALIAFLAFLIIVTSIALLVVLYKIYDLHK |
| | | KRSCNLDEQQELVERDDEKQLMNVEPIHADILLETYKRKIA |
| | | DEGRLFLAEFQSIPRVFSKFPIKEARKPFNONKNRYVDILPY |
| | | DYNRVELSEINGDAGSNYINASYIDGFKEPRKYIAAQGPRD |
| | | ETVDDFWRMIWEQKATVIVMVTRCEEGNRNKCAEYWPS |
| | | MEEGTRAFGDVVVKINQHKRCPDYIIQKLNIVNKKEKATG |
| | | REVTHIQFTSWPDHGVPEDPHLLLKLRRRVNAFSNFFSGPIV |
| | | VHCSAGVGRTGTYIGIDAMLEGLEAENKVDVYGYVVKLR |
| | | RQRCLMVQVEAQYILIHQALVEYNQFGETEVNLSELHPYL |
| | | HNMKKRDPPSEPSPLEAEFQRLPSYRSWRTQHIGNQEENKS |
| | | KNRNSNVIPYDYNRVPLKHELEMSKESEHDSDESSDDDSDS |
| | | EEPSKYINASFIMSYWKPEVMIAAQGPLKETIGDFWQMIFQ |
| | | RKVKVIVMLTELKHGDQEICAQYWGEGKQTYGDIEVDLK |
| | | DTDKSSTYTLRVFELRHSKRKDSRTVYQYQYTNWSVEQLP |
| | | AEPKELISMIQVVKQKLPQKNSSEGNKHHKSTPLLIHCRDG |
| | | SQQTGIFCALLNLLESAETEEVVDIFQVVKALRKARPGMVS |
| | | TFEQYQFLYDVIASTYPAQNGQVKKNNHQEDKIEFDNEVD |
| | | KVKQDANCVNPLGAPEKLPEAKEQAEGSEPTSGTEGPEHS |
| | | VNGPASPALNQGS*EKT*MRKLQTSC*LLFLFL*K*EVKIGI |
| | | QWIN*MQRTNICRRVIFYYCGKIFKIVLPEQFVQTYAYFKIL |
| | | SLIQ*KTTSL*SLCVYMYVCMGVCLCERQRKRENSFK*I*KL |
| | | LLFLCFYEEKIHFILEVLT*LEGSVFKNHKLCADSIKSCTFLK |
| | | *PQDVLLVLLIYIYLI*FTILLLEIVHKGGMCVYATTKKLLTK |
| | | LTLGNLIFHILAFSPMSF*AYLIKKFPVSLSCCLYMGFSILHA |
| | | RLFPVQYLKFIA*YF*QQIRFCTIELK*MSLK*INAICINIHCIK |
| | | IEEYKHIC*IFTYEI*YSYFYGIFH*YEKYDIAYA*FPC*IPFITF |
| | | IKAFTLNFSNA*NVFTRNGCR*S**NSTIIFFLFIIHCVICSNM |
| | | KCVCTY*NMFNAFINICRTLLQAPIIQ*SNNCLRSRKKKSRIV |
| | | VFFMK**KLVLV |
| 5 | tCD45RO_WT | MTMYLWLKLLAFGFAFLDTEVFVTGEQKLISEEDLQSPTPS |
| | AA | PTDAYLNASETTTLSPSGSAVISTTTIATTPSKPTCDEKYANI |
| | | TVDYLYNKETKLFTAKLNVNENVECGNNTCTNNEVHNLT |
| | | ECKNASVSISHNSCTAPDKTLILDVPPGVEKFQLHDCTQVE |
| | | KADTTICLKWKNIETFTCDTQNITYRFQCGNMIFDNKEIKLE |
| | | NLEPEHEYKCDSEILYNNHKFTNASKIIKTDFGSPGEPQIIFC |
| | | RSEAAHQGVITWNPPQRSFHNFTLCYIKETEKDCLNLDKNL |
| | | IKYDLQNLKPYTKYVLSLHAYIIAKVQRNGSAAMCHFTTK |
| | | SAPPSQVWNMTVSMTSDNSMHVKCRPPRDRNGPHERYHL |
| | | EVEAGNTLVRNESHKNCDFRVKDLQYSTDYTFKAYFHNG |
| | | DYPGEPFILHHSTSYNSKALIAFLAFLIIVTSIALLVVLYKIYD |
| | | L* |
| 6 | tCD45RO_WT | ATGACCATGTATTTGTGGCTTAAACTCTTGGCATTTGGCT |
| | | TTGCCTTTCTGGACACAGAAGTATTTGTGACAGGGGAAC |
| | | AAAAACTCATCTCAGAAGAGGATCTGCAAAGCCCAACA |
| | | CCTTCCCCCACTGATGCCTACCTTAATGCCTCTGAAACA |
| | | ACCACTCTGAGCCCTTCTGGAAGCGCTGTCATTTCAACC |
| | | ACAACAATAGCTACTACTCCATCTAAGCCAACATGTGAT |
| | | GAAAAATATGCAAACATCACTGTGGATTACTTATATAAC |
| | | AAGGAAACTAAATTATTTACAGCAAAGCTAAATGTTAAT |
| | | GAGAATGTGGAATGTGGAAACAATACTTGCACAAACAA |
| | | TGAGGTGCATAACCTTACAGAATGTAAAAATGCGTCTGT |
| | | TTCCATATCTCATAATTCATGTACTGCTCCTGATAAGACA |
| | | TTAATATTAGATGTGCCACCAGGGGTTGAAAAGTTTCAG |
| | | TTACATGATTGTACACAAGTTGAAAAAGCAGATACTACT |
| | | ATTTGTTTAAAATGGAAAAATATTGAAACCTTTACTTGT |
| | | GATACACAGAATATTACCTACAGATTTCAGTGTGGTAAT |
| | | ATGATATTTGATAATAAAGAAATTAAATTAGAAAACCTT |
| | | GAACCCGAACATGAGTATAAGTGTGACTCAGAAATACTC |
| | | TATAATAACCACAAGTTTACTAACGCAAGTAAAATTATT |
| | | AAAACAGATTTTGGGAGTCCAGGAGAGCCTCAGATTATT |
| | | TTTTGTAGAAGTGAAGCTGCACATCAAGGAGTAATTACC |
| | | TGGAATCCCCCTCAAAGATCATTTCATAATTTTACCCTCT |
| | | GTTATATAAAAGAGACAGAAAAAGATTGCCTCAATCTG |
| | | GATAAAAACCTGATCAAATATGATTTGCAAAATTTAAAA |
| | | CCTTATACGAAATATGTTTTATCATTACATGCCTACATCA |
| | | TTGCAAAAGTGCAACGTAATGGAAGTGCTGCAATGTGTC |
| | | ATTTCACAACTAAAAGTGCTCCTCCAAGCCAGGTCTGGA |
| | | ACATGACTGTCTCCATGACATCAGATAATAGTATGCATG |
| | | TCAAGTGTAGGCCTCCCAGGGACCGTAATGGCCCCCATG |
| | | AACGTTACCATTTGGAAGTTGAAGCTGGAAATACTCTGG |
| | | TTAGAAATGAGTCGCATAAGAATTGCGATTTCCGTGTAA |
| | | AAGATCTTCAATATTCAACAGACTACACTTTTAAGGCCT |
| | | ATTTTCACAATGGAGACTATCCTGGAGAACCCTTTATTTT |
| | | ACATCATTCAACATCTTATAATTCTAAGGCACTGATAGC |
| | | ATTTCTGGCATTTCTGATTATTGTGACATCAATAGCCCTG |
| | | CTTGTTGTTCTCTACAAAATCTATGATCTATAA |
| 7 | tCD45RO_- | ATGACCATGTATTTGTGGCTTAAACTCTTGGCATTTGGCT |
| | Mucin-like | TTGCCTTTCTGGACACAGAAGTATTTGTGACAGGGGAAC |
| | | AAAAACTCATCTCAGAAGAGGATCTGTGTGATGAAAAA |
| | | TATGCAAACATCACTGTGGATTACTTATATAACAAGGAA |
| | | ACTAAATTATTTACAGCAAAGCTAAATGTTAATGAGAAT |
| | | GTGGAATGTGGAAACAATACTTGCACAAACAATGAGGT |
| | | GCATAACCTTACAGAATGTAAAAATGCGTCTGTTTCCAT |
| | | ATCTCATAATTCATGTACTGCTCCTGATAAGACATTAAT |
| | | ATTAGATGTGCCACCAGGGGTTGAAAAGTTTCAGTTACA |
| | | TGATTGTACACAAGTTGAAAAAGCAGATACTACTATTTG |
| | | TTTAAAATGGAAAAATATTGAAACCTTTACTTGTGATAC |
| | | ACAGAATATTACCTACAGATTTCAGTGTGGTAATATGAT |
| | | ATTTGATAATAAAGAAATTAAATTAGAAAACCTTGAACC |
| | | CGAACATGAGTATAAGTGTGACTCAGAAATACTCTATAA |
| | | TAACCACAAGTTTACTAACGCAAGTAAAATTATTAAAAC |
| | | AGATTTTGGGAGTCCAGGAGAGCCTCAGATTATTTTTTG |
| | | TAGAAGTGAAGCTGCACATCAAGGAGTAATTACCTGGA |
| | | ATCCCCCTCAAAGATCATTTCATAATTTTACCCTCTGTTA |
| | | TATAAAAGAGACAGAAAAAGATTGCCTCAATCTGGATA |
| | | AAAACCTGATCAAATATGATTTGCAAAATTTAAAACCTT |
| | | ATACGAAATATGTTTTATCATTACATGCCTACATCATTGC |
| | | AAAAGTGCAACGTAATGGAAGTGCTGCAATGTGTCATTT |
| | | CACAACTAAAAGTGCTCCTCCAAGCCAGGTCTGGAACAT |
| | | GACTGTCTCCATGACATCAGATAATAGTATGCATGTCAA |
| | | GTGTAGGCCTCCCAGGGACCGTAATGGCCCCCATGAACG |
| | | TTACCATTTGGAAGTTGAAGCTGGAAATACTCTGGTTAG |
| | | AAATGAGTCGCATAAGAATTGCGATTTCCGTGTAAAAGA |
| | | TCTTCAATATTCAACAGACTACACTTTTAAGGCCTATTTT |
| | | CACAATGGAGACTATCCTGGAGAACCCTTTATTTTACAT |
| | | CATTCAACATCTTATAATTCTAAGGCACTGATAGCATTTC |
| | | TGGCATTTCTGATTATTGTGACATCAATAGCCCTGCTTGT |
| | | TGTTCTCTACAAAATCTATGATCTATAA |
| 8 | tCD45RO_- | MTMYLWLKLLAFGFAFLDTEVFVTGEQKLISEEDLCDEKY |
| | Mucin-like AA | ANITVDYLYNKETKLFTAKLNVNENVECGNNTCTNNEVH |
| | | NLTECKNASVSISHNSCTAPDKTLILDVPPGVEKFQLHDCT |
| | | QVEKADTTICLKWKNIETFTCDTQNITYRFQCGNMIFDNKE |
| | | IKLENLEPEHEYKCDSEILYNNHKFTNASKIIKTDFGSPGEP |
| | | QIIFCRSEAAHQGVITWNPPQRSFHNFTLCYIKETEKDCLNL |
| | | DKNLIKYDLQNLKPYTKYVLSLHAYIIAKVQRNGSAAMCH |
| | | FTTKSAPPSQVWNMTVSMTSDNSMHVKCRPPRDRNGPHE |
| | | RYHLEVEAGNTLVRNESHKNCDFRVKDLQYSTDYTFKAYF |
| | | HNGDYPGEPFILHHSTSYNSKALIAFLAFLIIVTSIALLVVLY |
| | | KIYDL* |
| 9 | tCD45RO_- | ATGACCATGTATTTGTGGCTTAAACTCTTGGCATTTGGCT |
| | Mucin-like-D1 | TTGCCTTTCTGGACACAGAAGTATTTGTGACAGGGGAAC |
| | | AAAAACTCATCTCAGAAGAGGATCTGCCACCAGGGGTT |
| | | GAAAAGTTTCAGTTACATGATTGTACACAAGTTGAAAAA |
| | | GCAGATACTACTATTTGTTTAAAATGGAAAAATATTGAA |
| | | ACCTTTACTTGTGATACACAGAATATTACCTACAGATTTC |
| | | AGTGTGGTAATATGATATTTGATAATAAAGAAATTAAAT |
| | | TAGAAAACCTTGAACCCGAACATGAGTATAAGTGTGACT |
| | | CAGAAATACTCTATAATAACCACAAGTTTACTAACGCAA |
| | | GTAAAATTATTAAAACAGATTTTGGGAGTCCAGGAGAGC |
| | | CTCAGATTATTTTTTGTAGAAGTGAAGCTGCACATCAAG |
| | | GAGTAATTACCTGGAATCCCCCTCAAAGATCATTTCATA |
| | | ATTTTACCCTCTGTTATATAAAAGAGACAGAAAAAGATT |
| | | GCCTCAATCTGGATAAAAACCTGATCAAATATGATTTGC |
| | | AAAATTTAAAACCTTATACGAAATATGTTTTATCATTAC |
| | | ATGCCTACATCATTGCAAAAGTGCAACGTAATGGAAGTG |
| | | CTGCAATGTGTCATTTCACAACTAAAAGTGCTCCTCCAA |
| | | GCCAGGTCTGGAACATGACTGTCTCCATGACATCAGATA |
| | | ATAGTATGCATGTCAAGTGTAGGCCTCCCAGGGACCGTA |
| | | ATGGCCCCCATGAACGTTACCATTTGGAAGTTGAAGCTG |
| | | GAAATACTCTGGTTAGAAATGAGTCGCATAAGAATTGCG |
| | | ATTTCCGTGTAAAAGATCTTCAATATTCAACAGACTACA |
| | | CTTTTAAGGCCTATTTTCACAATGGAGACTATCCTGGAG |
| | | AACCCTTTATTTTACATCATTCAACATCTTATAATTCTAA |
| | | GGCACTGATAGCATTTCTGGCATTTCTGATTATTGTGACA |
| | | TCAATAGCCCTGCTTGTTGTTCTCTACAAAATCTATGATC |
| | | TATAA |
| 10 | tCD45RO_- | MTMYLWLKLLAFGFAFLDTEVFVTGEQKLISEEDLPPGVE |
| | Mucin-like-D1 | KFQLHDCTQVEKADTTICLKWKNIETFTCDTQNITYRFQCG |
| | AA | NMIFDNKEIKLENLEPEHEYKCDSEILYNNHKFTNASKIIKT |
| | | DFGSPGEPQIIFCRSEAAHQGVITWNPPQRSFHNFTLCYIKE |
| | | TEKDCLNLDKNLIKYDLQNLKPYTKYVLSLHAYIIAKVQR |
| | | NGSAAMCHFTTKSAPPSQVWNMTVSMTSDNSMHVKCRPP |
| | | RDRNGPHERYHLEVEAGNTLVRNESHKNCDFRVKDLQYS |
| | | TDYTFKAYFHNGDYPGEPFILHHSTSYNSKALIAFLAFLIIVT |
| | | SIALLVVLYKIYDL* |
| 11 | tCD45RO_- | ATGACCATGTATTTGTGGCTTAAACTCTTGGCATTTGGCT |
| | Mucin-like-D1- | TTGCCTTTCTGGACACAGAAGTATTTGTGACAGGGGAAC |
| | D2 | AAAAACTCATCTCAGAAGAGGATCTGAGTCCAGGAGAG |
| | | CCTCAGATTATTTTTTGTAGAAGTGAAGCTGCACATCAA |
| | | GGAGTAATTACCTGGAATCCCCCTCAAAGATCATTTCAT |
| | | AATTTTACCCTCTGTTATATAAAAGAGACAGAAAAAGAT |
| | | TGCCTCAATCTGGATAAAAACCTGATCAAATATGATTTG |
| | | CAAAATTTAAAACCTTATACGAAATATGTTTTATCATTA |
| | | CATGCCTACATCATTGCAAAAGTGCAACGTAATGGAAGT |
| | | GCTGCAATGTGTCATTTCACAACTAAAAGTGCTCCTCCA |
| | | AGCCAGGTCTGGAACATGACTGTCTCCATGACATCAGAT |
| | | AATAGTATGCATGTCAAGTGTAGGCCTCCCAGGGACCGT |
| | | AATGGCCCCCATGAACGTTACCATTTGGAAGTTGAAGCT |
| | | GGAAATACTCTGGTTAGAAATGAGTCGCATAAGAATTGC |
| | | GATTTCCGTGTAAAAGATCTTCAATATTCAACAGACTAC |
| | | ACTTTTAAGGCCTATTTTCACAATGGAGACTATCCTGGA |
| | | GAACCCTTTATTTTACATCATTCAACATCTTATAATTCTA |
| | | AGGCACTGATAGCATTTCTGGCATTTCTGATTATTGTGAC |
| | | ATCAATAGCCCTGCTTGTTGTTCTCTACAAAATCTATGAT |
| | | CTATAA |
| 12 | tCD45RO_- | MTMYLWLKLLAFGFAFLDTEVFVTGEQKLISEEDLSPGEPQ |
| | Mucin-like-D1- | IIFCRSEAAHQGVITWNPPQRSFHNFTLCYIKETEKDCLNLD |
| | D2 AA | KNLIKYDLQNLKPYTKYVLSLHAYIIAKVQRNGSAAMCHF |
| | | TTKSAPPSQVWNMTVSMTSDNSMHVKCRPPRDRNGPHER |
| | | YHLEVEAGNTLVRNESHKNCDFRVKDLQYSTDYTFKAYF |
| | | HNGDYPGEPFILHHSTSYNSKALIAFLAFLIIVTSIALLVVLY |
| | | KIYDL* |
| 13 | tCD45RO_- | ATGACCATGTATTTGTGGCTTAAACTCTTGGCATTTGGCTTTG |
| | Mucin-like-D1- | CCTTTCTGGACACAGAAGTATTTGTGACAGGGGAACAAAAAC |
| | D2-D3 | TCATCTCAGAAGAGGATCTGCCTCCAAGCCAGGTCTGGAACA |
| | | TGACTGTCTCCATGACATCAGATAATAGTATGCATGTCAAGTG |
| | | TAGGCCTCCCAGGGACCGTAATGGCCCCCATGAACGTTACCA |
| | | TTTGGAAGTTGAAGCTGGAAATACTCTGGTTAGAAATGAGTC |
| | | GCATAAGAATTGCGATTTCCGTGTAAAAGATCTTCAATATTCA |
| | | ACAGACTACACTTTTAAGGCCTATTTTCACAATGGAGACTATC |
| | | CTGGAGAACCCTTTATTTTACATCATTCAACATCTTATAATTCT |
| | | AAGGCACTGATAGCATTTCTGGCATTTCTGATTATTGTGACAT |
| | | CAATAGCCCTGCTTGTTGTTCTCTACAAAATCTATGATCTATA |
| | | A |
| 14 | tCD45RO_- | MTMYLWLKLLAFGFAFLDTEVFVTGEQKLISEEDLPPSQVWNM |
| | Mucin-like-D1- | TVSMTSDNSMHVKCRPPRDRNGPHERYHLEVEAGNTLVRNESH |
| | D2-D3 AA | KNCDFRVKDLQYSTDYTFKAYFHNGDYPGEPFILHHSTSYNSKA |
| | | LIAFLAFLIIVTSIALLVVLYKIYDL* |
| 15 | tCD45_RO_D229- | ATGACCATGTATTTGTGGCTTAAACTCTTGGCATTTGGCTTTG |
| | Y232 | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGCAGCCGCGGCAGCAAACATCACT |
| | | GTGGATTACTTATATAACAAGGAAACTAAATTATTTACAGCA |
| | | AAGCTAAATGTTAATGAGAATGTGGAATGTGGAAACAATACT |
| | | TGCACAAACAATGAGGTGCATAACCTTACAGAATGTAAAAAT |
| | | GCGTCTGTTTCCATATCTCATAATTCATGTACTGCTCCTGATAA |
| | | GACATTAATATTAGATGTGCCACCAGGGGTTGAAAAGTTTCA |
| | | GTTACATGATTGTACACAAGTTGAAAAAGCAGATACTACTATT |
| | | TGTTTAAAATGGAAAAATATTGAAACCTTTACTTGTGATACAC |
| | | AGAATATTACCTACAGATTTCAGTGTGGTAATATGATATTTGA |
| | | TAATAAAGAAATTAAATTAGAAAACCTTGAACCCGAACATGA |
| | | GTATAAGTGTGACTCAGAAATACTCTATAATAACCACAAGTTT |
| | | ACTAACGCAAGTAAAATTATTAAAACAGATTTTGGGAGTCCA |
| | | GGAGAGCCTCAGATTATTTTTTGTAGAAGTGAAGCTGCACATC |
| | | AAGGAGTAATTACCTGGAATCCCCCTCAAAGATCATTTCATAA |
| | | TTTTACCCTCTGTTATATAAAAGAGACAGAAAAAGATTGCCTC |
| | | AATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 16 | tCD45_RO_D229- | MTMYLWLKLLAFGFAFLDTEVFVTGEQKLISEEDLQSPTPSPTDA |
| | Y232 AA | YLNASETTTLSPSGSAVISTTTIATTPSKPTCAAAAANITVDYLYN |
| | | KETKLFTAKLNVNENVECGNNTCTNNEVHNLTECKNASVSISHN |
| | | SCTAPDKTLILDVPPGVEKFQLHDCTQVEKADTTICLKWKNIETF |
| | | TCDTQNITYRFQCGNMIFDNKEIKLENLEPEHEYKCDSEILYNNH |
| | | KFTNASKIIKTDFGSPGEPQIIFCRSEAAHQGVITWNPPQRSFHNFT |
| | | LCYIKETEKDCLNLDKNLIKYDLQNLKPYTKYVLSLHAYIIAKVQ |
| | | RNGSAAMCHFTTKSAPPSQVWNMTVSMTSDNSMHVKCRPPRD |
| | | RNGPHERYHLEVEAGNTLVRNESHKNCDFRVKDLQYSTDYTFK |
| | | AYFHNGDYPGEPFILHHSTSYNSKALIAFLAFLIIVTSIALLVVLYK |
| | | IYDL* |
| 17 | tCD45_RO_V258, | ATGACCATGTATTTGTGGCTTAAACTCTTGGCATTTGGCTTTG |
| | E259 | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGAAAAATATGCAAACATCACT |
| | | GTGGATTACTTATATAACAAGGAAACTAAATTATTTACAGCA |
| | | AAGCTAAATGTTAATGAGAATGCGGCCTGTGGAAACAATACT |
| | | TGCACAAACAATGAGGTGCATAACCTTACAGAATGTAAAAAT |
| | | GCGTCTGTTTCCATATCTCATAATTCATGTACTGCTCCTGATAA |
| | | GACATTAATATTAGATGTGCCACCAGGGGTTGAAAAGTTTCA |
| | | GTTACATGATTGTACACAAGTTGAAAAAGCAGATACTACTATT |
| | | TGTTTAAAATGGAAAAATATTGAAACCTTTACTTGTGATACAC |
| | | AGAATATTACCTACAGATTTCAGTGTGGTAATATGATATTTGA |
| | | TAATAAAGAAATTAAATTAGAAAACCTTGAACCCGAACATGA |
| | | GTATAAGTGTGACTCAGAAATACTCTATAATAACCACAAGTTT |
| | | ACTAACGCAAGTAAAATTATTAAAACAGATTTTGGGAGTCCA |
| | | GGAGAGCCTCAGATTATTTTTTGTAGAAGTGAAGCTGCACATC |
| | | AAGGAGTAATTACCTGGAATCCCCCTCAAAGATCATTTCATAA |
| | | TTTTACCCTCTGTTATATAAAAGAGACAGAAAAAGATTGCCTC |
| | | AATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 18 | tCD45_RO_V258, | MTMYLWLKLLAFGFAFLDTEVFVTGEQKLISEEDLQSPTPSPTDA |
| | E259 | YLNASETTTLSPSGSAVISTTTIATTPSKPTCDEKYANITVDYLYN |
| | | KETKLFTAKLNVNENAACGNNTCTNNEVHNLTECKNASVSISHN |
| | | SCTAPDKTLILDVPPGVEKFQLHDCTQVEKADTTICLKWKNIETF |
| | | TCDTQNITYRFQCGNMIFDNKEIKLENLEPEHEYKCDSEILYNNH |
| | | KFTNASKIIKTDFGSPGEPQIIFCRSEAAHQGVITWNPPQRSFHNFT |
| | | LCYIKETEKDCLNLDKNLIKYDLQNLKPYTKYVLSLHAYIIAKVQ |
| | | RNGSAAMCHFTTKSAPPSQVWNMTVSMTSDNSMHVKCRPPRD |
| | | RNGPHERYHLEVEAGNTLVRNESHKNCDFRVKDLQYSTDYTFK |
| | | AYFHNGDYPGEPFILHHSTSYNSKALIAFLAFLIIVTSIALLVVLYK |
| | | IYDL* |
| 19 | tCD45_RO_T289, | ATGACCATGTATTTGTGGCTTAAACTCTTGGCATTTGGCTTTG |
| | D292, K293 | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGAAAAATATGCAAACATCACT |
| | | GTGGATTACTTATATAACAAGGAAACTAAATTATTTACAGCA |
| | | AAGCTAAATGTTAATGAGAATGTGGAATGTGGAAACAATACT |
| | | TGCACAAACAATGAGGTGCATAACCTTACAGAATGTAAAAAT |
| | | GCGTCTGTTTCCATATCTCATAATTCATGTGCCGCTCCTGCTGC |
| | | GACATTAATATTAGATGTGCCACCAGGGGTTGAAAAGTTTCA |
| | | GTTACATGATTGTACACAAGTTGAAAAAGCAGATACTACTATT |
| | | TGTTTAAAATGGAAAAATATTGAAACCTTTACTTGTGATACAC |
| | | AGAATATTACCTACAGATTTCAGTGTGGTAATATGATATTTGA |
| | | TAATAAAGAAATTAAATTAGAAAACCTTGAACCCGAACATGA |
| | | GTATAAGTGTGACTCAGAAATACTCTATAATAACCACAAGTTT |
| | | ACTAACGCAAGTAAAATTATTAAAACAGATTTTGGGAGTCCA |
| | | GGAGAGCCTCAGATTATTTTTTGTAGAAGTGAAGCTGCACATC |
| | | AAGGAGTAATTACCTGGAATCCCCCTCAAAGATCATTTCATAA |
| | | TTTTACCCTCTGTTATATAAAAGAGACAGAAAAAGATTGCCTC |
| | | AATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 20 | tCD45_RO_T289, | MTMYLWLKLLAFGFAFLDTEVFVTGEQKLISEEDLQSPTPSPTDA |
| | D292, K293 AA | YLNASETTTLSPSGSAVISTTTIATTPSKPTCDEKYANITVDYLYN |
| | | KETKLFTAKLNVNENVECGNNTCTNNEVHNLTECKNASVSISHN |
| | | SCAAPAATLILDVPPGVEKFQLHDCTQVEKADTTICLKWKNIETF |
| | | TCDTQNITYRFQCGNMIFDNKEIKLENLEPEHEYKCDSEILYNNH |
| | | KFTNASKIIKTDFGSPGEPQIIFCRSEAAHQGVITWNPPQRSFHNFT |
| | | LCYIKETEKDCLNLDKNLIKYDLQNLKPYTKYVLSLHAYIIAKVQ |
| | | RNGSAAMCHFTTKSAPPSQVWNMTVSMTSDNSMHVKCRPPRD |
| | | RNGPHERYHLEVEAGNTLVRNESHKNCDFRVKDLQYSTDYTFK |
| | | AYFHNGDYPGEPFILHHSTSYNSKALIAFLAFLIIVTSIALLVVLYK |
| | | IYDL* |
| 21 | tCD45_RO_T274 | ATGACCATGTATTTGTGGCTTAAACTCTTGGCATTTGGCTTTG |
| | | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGAAAAATATGCAAACATCACT |
| | | GTGGATTACTTATATAACAAGGAAACTAAATTATTTACAGCA |
| | | AAGCTAAATGTTAATGAGAATGTGGAATGTGGAAACAATACT |
| | | TGCACAAACAATGAGGTGCATAACCTTGCCGAATGTAAAAAT |
| | | GCGTCTGTTTCCATATCTCATAATTCATGTACTGCTCCTGATAA |
| | | GACATTAATATTAGATGTGCCACCAGGGGTTGAAAAGTTTCA |
| | | GTTACATGATTGTACACAAGTTGAAAAAGCAGATACTACTATT |
| | | TGTTTAAAATGGAAAAATATTGAAACCTTTACTTGTGATACAC |
| | | AGAATATTACCTACAGATTTCAGTGTGGTAATATGATATTTGA |
| | | TAATAAAGAAATTAAATTAGAAAACCTTGAACCCGAACATGA |
| | | GTATAAGTGTGACTCAGAAATACTCTATAATAACCACAAGTTT |
| | | ACTAACGCAAGTAAAATTATTAAAACAGATTTTGGGAGTCCA |
| | | GGAGAGCCTCAGATTATTTTTTGTAGAAGTGAAGCTGCACATC |
| | | AAGGAGTAATTACCTGGAATCCCCCTCAAAGATCATTTCATAA |
| | | TTTTACCCTCTGTTATATAAAAGAGACAGAAAAAGATTGCCTC |
| | | AATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 22 | tCD45_RO_T274 | MTMYLWLKLLAFGFAFLDTEVFVTGEQKLISEEDLQSPTPSPTDA |
| | AA | YLNASETTTLSPSGSAVISTTTIATTPSKPTCDEKYANITVDYLYN |
| | | KETKLFTAKLNVNENVECGNNTCTNNEVHNLAECKNASVSISHN |
| | | SCTAPDKTLILDVPPGVEKFQLHDCTQVEKADTTICLKWKNIETF |
| | | TCDTQNITYRFQCGNMIFDNKEIKLENLEPEHEYKCDSEILYNNH |
| | | KFTNASKIIKTDFGSPGEPQIIFCRSEAAHQGVITWNPPQRSFHNFT |
| | | LCYIKETEKDCLNLDKNLIKYDLQNLKPYTKYVLSLHAYIIAKVQ |
| | | RNGSAAMCHFTTKSAPPSQVWNMTVSMTSDNSMHVKCRPPRD |
| | | RNGPHERYHLEVEAGNTLVRNESHKNCDFRVKDLQYSTDYTFK |
| | | AYFHNGDYPGEPFILHHSTSYNSKALIAFLAFLIIVTSIALLVVLYK |
| | | IYDL* |
| 23 | tCD45_RO_T266, | ATGACCATGTATTTGTGGCTTAAACTCTTGGCATTTGGCTTTG |
| | N267 | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGAAAAATATGCAAACATCACT |
| | | GTGGATTACTTATATAACAAGGAAACTAAATTATTTACAGCA |
| | | AAGCTAAATGTTAATGAGAATGTGGAATGTGGAAACAATACT |
| | | TGCGCCGCTAATGAGGTGCATAACCTTACAGAATGTAAAAAT |
| | | GCGTCTGTTTCCATATCTCATAATTCATGTACTGCTCCTGATAA |
| | | GACATTAATATTAGATGTGCCACCAGGGGTTGAAAAGTTTCA |
| | | GTTACATGATTGTACACAAGTTGAAAAAGCAGATACTACTATT |
| | | TGTTTAAAATGGAAAAATATTGAAACCTTTACTTGTGATACAC |
| | | AGAATATTACCTACAGATTTCAGTGTGGTAATATGATATTTGA |
| | | TAATAAAGAAATTAAATTAGAAAACCTTGAACCCGAACATGA |
| | | GTATAAGTGTGACTCAGAAATACTCTATAATAACCACAAGTTT |
| | | ACTAACGCAAGTAAAATTATTAAAACAGATTTTGGGAGTCCA |
| | | GGAGAGCCTCAGATTATTTTTTGTAGAAGTGAAGCTGCACATC |
| | | AAGGAGTAATTACCTGGAATCCCCCTCAAAGATCATTTCATAA |
| | | TTTTACCCTCTGTTATATAAAAGAGACAGAAAAAGATTGCCTC |
| | | AATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 24 | tCD45_RO_T266, | MTMYLWLKLLAFGFAFLDTEVFVTGEQKLISEEDLQSPTPSPTDA |
| | N267 AA | YLNASETTTLSPSGSAVISTTTIATTPSKPTCDEKYANITVDYLYN |
| | | KETKLFTAKLNVNENVECGNNTCAANEVHNLTECKNASVSISHN |
| | | SCTAPDKTLILDVPPGVEKFQLHDCTQVEKADTTICLKWKNIETF |
| | | TCDTQNITYRFQCGNMIFDNKEIKLENLEPEHEYKCDSEILYNNH |
| | | KFTNASKIIKTDFGSPGEPQIIFCRSEAAHQGVITWNPPQRSFHNFT |
| | | LCYIKETEKDCLNLDKNLIKYDLQNLKPYTKYVLSLHAYIIAKVQ |
| | | RNGSAAMCHFTTKSAPPSQVWNMTVSMTSDNSMHVKCRPPRD |
| | | RNGPHERYHLEVEAGNTLVRNESHKNCDFRVKDLQYSTDYTFK |
| | | AYFHNGDYPGEPFILHHSTSYNSKALIAFLAFLIIVTSIALLVVLYK |
| | | IYDL* |
| 25 | tCD45RO_T249, | ATGACCATGTATTTGTGGCTTAAACTCTTGGCATTTGGCTTTG |
| | K251 | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGAAAAATATGCAAACATCACT |
| | | GTGGATTACTTATATAACAAGGAAACTAAATTATTTGCCGCAG |
| | | CTCTAAATGTTAATGAGAATGTGGAATGTGGAAACAATACTT |
| | | GCACAAACAATGAGGTGCATAACCTTACAGAATGTAAAAATG |
| | | CGTCTGTTTCCATATCTCATAATTCATGTACTGCTCCTGATAAG |
| | | ACATTAATATTAGATGTGCCACCAGGGGTTGAAAAGTTTCAGT |
| | | TACATGATTGTACACAAGTTGAAAAAGCAGATACTACTATTTG |
| | | TTTAAAATGGAAAAATATTGAAACCTTTACTTGTGATACACAG |
| | | AATATTACCTACAGATTTCAGTGTGGTAATATGATATTTGATA |
| | | ATAAAGAAATTAAATTAGAAAACCTTGAACCCGAACATGAGT |
| | | ATAAGTGTGACTCAGAAATACTCTATAATAACCACAAGTTTAC |
| | | TAACGCAAGTAAAATTATTAAAACAGATTTTGGGAGTCCAGG |
| | | AGAGCCTCAGATTATTTTTTGTAGAAGTGAAGCTGCACATCAA |
| | | GGAGTAATTACCTGGAATCCCCCTCAAAGATCATTTCATAATT |
| | | TTACCCTCTGTTATATAAAAGAGACAGAAAAAGATTGCCTCA |
| | | ATCTGGATAAAAACCTGATCAAATATGATTTGCAAAATTTAA |
| | | AACCTTATACGAAATATGTTTTATCATTACATGCCTACATCAT |
| | | TGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 26 | tCD45RO_T249, | MTMYLWLKLLAFGFAFLDTEVFVTGEQKLISEEDLQSPTPSPTDA |
| | K251 AA | YLNASETTTLSPSGSAVISTTTIATTPSKPTCDEKYANITVDYLYN |
| | | KETKLFAAALNVNENVECGNNTCTNNEVHNLTECKNASVSISHN |
| | | SCTAPDKTLILDVPPGVEKFQLHDCTQVEKADTTICLKWKNIETF |
| | | TCDTQNITYRFQCGNMIFDNKEIKLENLEPEHEYKCDSEILYNNH |
| | | KFTNASKIIKTDFGSPGEPQIIFCRSEAAHQGVITWNPPQRSFHNFT |
| | | LCYIKETEKDCLNLDKNLIKYDLQNLKPYTKYVLSLHAYIIAKVQ |
| | | RNGSAAMCHFTTKSAPPSQVWNMTVSMTSDNSMHVKCRPPRD |
| | | RNGPHERYHLEVEAGNTLVRNESHKNCDFRVKDLQYSTDYTFK |
| | | AYFHNGDYPGEPFILHHSTSYNSKALIAFLAFLIIVTSIALLVVLYK |
| | | IYDL* |
| 27 | tCD45RO_T245- | ATGACCATGTATTTGTGGCTTAAACTCTTGGCATTTGGCTTTG |
| | L247 | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGAAAAATATGCAAACATCACT |
| | | GTGGATTACTTATATAACAAGGAAGCCGCGGCTTTTACAGCA |
| | | AAGCTAAATGTTAATGAGAATGTGGAATGTGGAAACAATACT |
| | | TGCACAAACAATGAGGTGCATAACCTTACAGAATGTAAAAAT |
| | | GCGTCTGTTTCCATATCTCATAATTCATGTACTGCTCCTGATAA |
| | | GACATTAATATTAGATGTGCCACCAGGGGTTGAAAAGTTTCA |
| | | GTTACATGATTGTACACAAGTTGAAAAAGCAGATACTACTATT |
| | | TGTTTAAAATGGAAAAATATTGAAACCTTTACTTGTGATACAC |
| | | AGAATATTACCTACAGATTTCAGTGTGGTAATATGATATTTGA |
| | | TAATAAAGAAATTAAATTAGAAAACCTTGAACCCGAACATGA |
| | | GTATAAGTGTGACTCAGAAATACTCTATAATAACCACAAGTTT |
| | | ACTAACGCAAGTAAAATTATTAAAACAGATTTTGGGAGTCCA |
| | | GGAGAGCCTCAGATTATTTTTTGTAGAAGTGAAGCTGCACATC |
| | | AAGGAGTAATTACCTGGAATCCCCCTCAAAGATCATTTCATAA |
| | | TTTTACCCTCTGTTATATAAAAGAGACAGAAAAAGATTGCCTC |
| | | AATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 28 | tCD45RO_T245- | MTMYLWLKLLAFGFAFLDTEVFVTGEQKLISEEDLQSPTPSPTDA |
| | L247 AA | YLNASETTTLSPSGSAVISTTTIATTPSKPTCDEKYANITVDYLYN |
| | | KEAAAFTAKLNVNENVECGNNTCTNNEVHNLTECKNASVSISH |
| | | NSCTAPDKTLILDVPPGVEKFQLHDCTQVEKADTTICLKWKNIET |
| | | FTCDTQNITYRFQCGNMIFDNKEIKLENLEPEHEYKCDSEILYNN |
| | | HKFTNASKIIKTDFGSPGEPQIIFCRSEAAHQGVITWNPPQRSFHN |
| | | FTLCYIKETEKDCLNLDKNLIKYDLQNLKPYTKYVLSLHAYIIAK |
| | | VQRNGSAAMCHFTTKSAPPSQVWNMTVSMTSDNSMHVKCRPP |
| | | RDRNGPHERYHLEVEAGNTLVRNESHKNCDFRVKDLQYSTDYT |
| | | FKAYFHNGDYPGEPFILHHSTSYNSKALIAFLAFLIIVTSIALLVVL |
| | | YKIYDL* |
| 29 | tCD45_RO_T8A | ATGACCATGTATTTGTGGCTTAAACTCTTGGCATTTGGCTTTG |
| | | CCTTTCTGGACACAGAAGTATTTGTGACAGGGGAACAAAAAC |
| | | TCATCTCAGAAGAGGATCTGCAAAGCCCAACACCTTCCCCCG |
| | | CGGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGAAAAATATGCAAACATCACT |
| | | GTGGATTACTTATATAACAAGGAAACTAAATTATTTACAGCA |
| | | AAGCTAAATGTTAATGAGAATGTGGAATGTGGAAACAATACT |
| | | TGCACAAACAATGAGGTGCATAACCTTACAGAATGTAAAAAT |
| | | GCGTCTGTTTCCATATCTCATAATTCATGTACTGCTCCTGATAA |
| | | GACATTAATATTAGATGTGCCACCAGGGGTTGAAAAGTTTCA |
| | | GTTACATGATTGTACACAAGTTGAAAAAGCAGATACTACTATT |
| | | TGTTTAAAATGGAAAAATATTGAAACCTTTACTTGTGATACAC |
| | | AGAATATTACCTACAGATTTCAGTGTGGTAATATGATATTTGA |
| | | TAATAAAGAAATTAAATTAGAAAACCTTGAACCCGAACATGA |
| | | GTATAAGTGTGACTCAGAAATACTCTATAATAACCACAAGTTT |
| | | ACTAACGCAAGTAAAATTATTAAAACAGATTTTGGGAGTCCA |
| | | GGAGAGCCTCAGATTATTTTTTGTAGAAGTGAAGCTGCACATC |
| | | AAGGAGTAATTACCTGGAATCCCCCTCAAAGATCATTTCATAA |
| | | TTTTACCCTCTGTTATATAAAAGAGACAGAAAAAGATTGCCTC |
| | | AATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 30 | tCD45_RO_T8A | MTMYLWLKLLAFGFAFLDTEVFVTGEQKLISEEDLQSPTPSPAD |
| | AA | AYLNASETTTLSPSGSAVISTTTIATTPSKPTCDEKYANITVDYLY |
| | | NKETKLFTAKLNVNENVECGNNTCTNNEVHNLTECKNASVSISH |
| | | NSCTAPDKTLILDVPPGVEKFQLHDCTQVEKADTTICLKWKNIET |
| | | FTCDTQNITYRFQCGNMIFDNKEIKLENLEPEHEYKCDSEILYNN |
| | | HKFTNASKIIKTDFGSPGEPQIIFCRSEAAHQGVITWNPPQRSFHN |
| | | FTLCYIKETEKDCLNLDKNLIKYDLQNLKPYTKYVLSLHAYIIAK |
| | | VQRNGSAAMCHFTTKSAPPSQVWNMTVSMTSDNSMHVKCRPP |
| | | RDRNGPHERYHLEVEAGNTLVRNESHKNCDFRVKDLQYSTDYT |
| | | FKAYFHNGDYPGEPFILHHSTSYNSKALIAFLAFLIIVTSIALLVVL |
| | | YKIYDL* |
| 31 | tCD45_RO_N263, | ATGACCATGTATTTGTGGCTTAAACTCTTGGCATTTGGCTTTG |
| | T264 | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGAAAAATATGCAAACATCACT |
| | | GTGGATTACTTATATAACAAGGAAACTAAATTATTTACAGCA |
| | | AAGCTAAATGTTAATGAGAATGTGGAATGTGGAAACGCTGCG |
| | | TGCACAAACAATGAGGTGCATAACCTTACAGAATGTAAAAAT |
| | | GCGTCTGTTTCCATATCTCATAATTCATGTACTGCTCCTGATAA |
| | | GACATTAATATTAGATGTGCCACCAGGGGTTGAAAAGTTTCA |
| | | GTTACATGATTGTACACAAGTTGAAAAAGCAGATACTACTATT |
| | | TGTTTAAAATGGAAAAATATTGAAACCTTTACTTGTGATACAC |
| | | AGAATATTACCTACAGATTTCAGTGTGGTAATATGATATTTGA |
| | | TAATAAAGAAATTAAATTAGAAAACCTTGAACCCGAACATGA |
| | | GTATAAGTGTGACTCAGAAATACTCTATAATAACCACAAGTTT |
| | | ACTAACGCAAGTAAAATTATTAAAACAGATTTTGGGAGTCCA |
| | | GGAGAGCCTCAGATTATTTTTTGTAGAAGTGAAGCTGCACATC |
| | | AAGGAGTAATTACCTGGAATCCCCCTCAAAGATCATTTCATAA |
| | | TTTTACCCTCTGTTATATAAAAGAGACAGAAAAAGATTGCCTC |
| | | AATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 32 | tCD45_RO_N263, | MTMYLWLKLLAFGFAFLDTEVFVTGEQKLISEEDLQSPTPSPTDA |
| | T264 AA | YLNASETTTLSPSGSAVISTTTIATTPSKPTCDEKYANITVDYLYN |
| | | KETKLFTAKLNVNENVECGNAACTNNEVHNLTECKNASVSISHN |
| | | SCTAPDKTLILDVPPGVEKFQLHDCTQVEKADTTICLKWKNIETF |
| | | TCDTQNITYRFQCGNMIFDNKEIKLENLEPEHEYKCDSEILYNNH |
| | | KFTNASKIIKTDFGSPGEPQIIFCRSEAAHQGVITWNPPQRSFHNFT |
| | | LCYIKETEKDCLNLDKNLIKYDLQNLKPYTKYVLSLHAYIIAKVQ |
| | | RNGSAAMCHFTTKSAPPSQVWNMTVSMTSDNSMHVKCRPPRD |
| | | RNGPHERYHLEVEAGNTLVRNESHKNCDFRVKDLQYSTDYTFK |
| | | AYFHNGDYPGEPFILHHSTSYNSKALIAFLAFLIIVTSIALLVVLYK |
| | | IYDL* |
| 33 | tCD45_RO_N253, | ATGACCATGTATTTGTGGCTTAAACTCTTGGCATTTGGCTTTG |
| | N255, E256 | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGAAAAATATGCAAACATCACT |
| | | GTGGATTACTTATATAACAAGGAAACTAAATTATTTACAGCA |
| | | AAGCTAGCGGTTGCCGCTAATGTGGAATGTGGAAACAATACT |
| | | TGCACAAACAATGAGGTGCATAACCTTACAGAATGTAAAAAT |
| | | GCGTCTGTTTCCATATCTCATAATTCATGTACTGCTCCTGATAA |
| | | GACATTAATATTAGATGTGCCACCAGGGGTTGAAAAGTTTCA |
| | | GTTACATGATTGTACACAAGTTGAAAAAGCAGATACTACTATT |
| | | TGTTTAAAATGGAAAAATATTGAAACCTTTACTTGTGATACAC |
| | | AGAATATTACCTACAGATTTCAGTGTGGTAATATGATATTTGA |
| | | TAATAAAGAAATTAAATTAGAAAACCTTGAACCCGAACATGA |
| | | GTATAAGTGTGACTCAGAAATACTCTATAATAACCACAAGTTT |
| | | ACTAACGCAAGTAAAATTATTAAAACAGATTTTGGGAGTCCA |
| | | GGAGAGCCTCAGATTATTTTTTGTAGAAGTGAAGCTGCACATC |
| | | AAGGAGTAATTACCTGGAATCCCCCTCAAAGATCATTTCATAA |
| | | TTTTACCCTCTGTTATATAAAAGAGACAGAAAAAGATTGCCTC |
| | | AATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 34 | tCD45_RO_N253, | MTMYLWLKLLAFGFAFLDTEVFVTGEQKLISEEDLQSPTPSPTDA |
| | N255, E256 AA | YLNASETTTLSPSGSAVISTTTIATTPSKPTCDEKYANITVDYLYN |
| | | KETKLFTAKLAVAANVECGNNTCTNNEVHNLTECKNASVSISHN |
| | | SCTAPDKTLILDVPPGVEKFQLHDCTQVEKADTTICLKWKNIETF |
| | | TCDTQNITYRFQCGNMIFDNKEIKLENLEPEHEYKCDSEILYNNH |
| | | KFTNASKIIKTDFGSPGEPQIIFCRSEAAHQGVITWNPPQRSFHNFT |
| | | LCYIKETEKDCLNLDKNLIKYDLQNLKPYTKYVLSLHAYIIAKVQ |
| | | RNGSAAMCHFTTKSAPPSQVWNMTVSMTSDNSMHVKCRPPRD |
| | | RNGPHERYHLEVEAGNTLVRNESHKNCDFRVKDLQYSTDYTFK |
| | | AYFHNGDYPGEPFILHHSTSYNSKALIAFLAFLIIVTSIALLVVLYK |
| | | IYDL |
| 35 | tCD45_RO_N242- | ATGACCATGTATTTGTGGCTTAAACTCTTGGCATTTGGCTTTG |
| | E244 | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGAAAAATATGCAAACATCACT |
| | | GTGGATTACTTATATGCCGCGGCTACTAAATTATTTACAGCAA |
| | | AGCTAAATGTTAATGAGAATGTGGAATGTGGAAACAATACTT |
| | | GCACAAACAATGAGGTGCATAACCTTACAGAATGTAAAAATG |
| | | CGTCTGTTTCCATATCTCATAATTCATGTACTGCTCCTGATAAG |
| | | ACATTAATATTAGATGTGCCACCAGGGGTTGAAAAGTTTCAGT |
| | | TACATGATTGTACACAAGTTGAAAAAGCAGATACTACTATTTG |
| | | TTTAAAATGGAAAAATATTGAAACCTTTACTTGTGATACACAG |
| | | AATATTACCTACAGATTTCAGTGTGGTAATATGATATTTGATA |
| | | ATAAAGAAATTAAATTAGAAAACCTTGAACCCGAACATGAGT |
| | | ATAAGTGTGACTCAGAAATACTCTATAATAACCACAAGTTTAC |
| | | TAACGCAAGTAAAATTATTAAAACAGATTTTGGGAGTCCAGG |
| | | AGAGCCTCAGATTATTTTTTGTAGAAGTGAAGCTGCACATCAA |
| | | GGAGTAATTACCTGGAATCCCCCTCAAAGATCATTTCATAATT |
| | | TTACCCTCTGTTATATAAAAGAGACAGAAAAAGATTGCCTCA |
| | | ATCTGGATAAAAACCTGATCAAATATGATTTGCAAAATTTAA |
| | | AACCTTATACGAAATATGTTTTATCATTACATGCCTACATCAT |
| | | TGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 36 | tCD45_RO_N242- | MTMYLWLKLLAFGFAFLDTEVFVTGEQKLISEEDLQSPTPSPTDA |
| | E244 AA | YLNASETTTLSPSGSAVISTTTIATTPSKPTCDEKYANITVDYLYA |
| | | AATKLFTAKLNVNENVECGNNTCTNNEVHNLTECKNASVSISHN |
| | | SCTAPDKTLILDVPPGVEKFQLHDCTQVEKADTTICLKWKNIETF |
| | | TCDTQNITYRFQCGNMIFDNKEIKLENLEPEHEYKCDSEILYNNH |
| | | KFTNASKIIKTDFGSPGEPQIIFCRSEAAHQGVITWNPPQRSFHNFT |
| | | LCYIKETEKDCLNLDKNLIKYDLQNLKPYTKYVLSLHAYIIAKVQ |
| | | RNGSAAMCHFTTKSAPPSQVWNMTVSMTSDNSMHVKCRPPRD |
| | | RNGPHERYHLEVEAGNTLVRNESHKNCDFRVKDLQYSTDYTFK |
| | | AYFHNGDYPGEPFILHHSTSYNSKALIAFLAFLIIVTSIALLVVLYK |
| | | IYDL* |
| 37 | tCD45RO_L295, | ATGACCATGTATTTGTGGCTTAAACTCTTGGCATTTGGCTTTG |
| | I296, D298 | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGAAAAATATGCAAACATCACT |
| | | GTGGATTACTTATATAACAAGGAAACTAAATTATTTACAGCA |
| | | AAGCTAAATGTTAATGAGAATGTGGAATGTGGAAACAATACT |
| | | TGCACAAACAATGAGGTGCATAACCTTACAGAATGTAAAAAT |
| | | GCGTCTGTTTCCATATCTCATAATTCATGTACTGCTCCTGATAA |
| | | GACAGCGGCTTTAGCCGTGCCACCAGGGGTTGAAAAGTTTCA |
| | | GTTACATGATTGTACACAAGTTGAAAAAGCAGATACTACTATT |
| | | TGTTTAAAATGGAAAAATATTGAAACCTTTACTTGTGATACAC |
| | | AGAATATTACCTACAGATTTCAGTGTGGTAATATGATATTTGA |
| | | TAATAAAGAAATTAAATTAGAAAACCTTGAACCCGAACATGA |
| | | GTATAAGTGTGACTCAGAAATACTCTATAATAACCACAAGTTT |
| | | ACTAACGCAAGTAAAATTATTAAAACAGATTTTGGGAGTCCA |
| | | GGAGAGCCTCAGATTATTTTTTGTAGAAGTGAAGCTGCACATC |
| | | AAGGAGTAATTACCTGGAATCCCCCTCAAAGATCATTTCATAA |
| | | TTTTACCCTCTGTTATATAAAAGAGACAGAAAAAGATTGCCTC |
| | | AATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 38 | tCD45RO_L295, | MTMYLWLKLLAFGFAFLDTEVFVTGEQKLISEEDLQSPTPSPTDA |
| | I296, D298 AA | YLNASETTTLSPSGSAVISTTTIATTPSKPTCDEKYANITVDYLYN |
| | | KETKLFTAKLNVNENVECGNNTCTNNEVHNLTECKNASVSISHN |
| | | SCTAPDKTAALAVPPGVEKFQLHDCTQVEKADTTICLKWKNIET |
| | | FTCDTQNITYRFQCGNMIFDNKEIKLENLEPEHEYKCDSEILYNN |
| | | HKFTNASKIIKTDFGSPGEPQIIFCRSEAAHQGVITWNPPQRSFHN |
| | | FTLCYIKETEKDCLNLDKNLIKYDLQNLKPYTKYVLSLHAYIIAK |
| | | VQRNGSAAMCHFTTKSAPPSQVWNMTVSMTSDNSMHVKCRPP |
| | | RDRNGPHERYHLEVEAGNTLVRNESHKNCDFRVKDLQYSTDYT |
| | | FKAYFHNGDYPGEPFILHHSTSYNSKALIAFLAFLIIVTSIALLVVL |
| | | YKIYDL* |
| 39 | tCD45_RO_K277, | ATGACCATGTATTTGTGGCTTAAACTCTTGGCATTTGGCTTTG |
| | N278, S280 | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGAAAAATATGCAAACATCACT |
| | | GTGGATTACTTATATAACAAGGAAACTAAATTATTTACAGCA |
| | | AAGCTAAATGTTAATGAGAATGTGGAATGTGGAAACAATACT |
| | | TGCACAAACAATGAGGTGCATAACCTTACAGAATGTGCCGCT |
| | | GCGGCCGTTTCCATATCTCATAATTCATGTACTGCTCCTGATA |
| | | AGACATTAATATTAGATGTGCCACCAGGGGTTGAAAAGTTTC |
| | | AGTTACATGATTGTACACAAGTTGAAAAAGCAGATACTACTA |
| | | TTTGTTTAAAATGGAAAAATATTGAAACCTTTACTTGTGATAC |
| | | ACAGAATATTACCTACAGATTTCAGTGTGGTAATATGATATTT |
| | | GATAATAAAGAAATTAAATTAGAAAACCTTGAACCCGAACAT |
| | | GAGTATAAGTGTGACTCAGAAATACTCTATAATAACCACAAG |
| | | TTTACTAACGCAAGTAAAATTATTAAAACAGATTTTGGGAGTC |
| | | CAGGAGAGCCTCAGATTATTTTTTGTAGAAGTGAAGCTGCAC |
| | | ATCAAGGAGTAATTACCTGGAATCCCCCTCAAAGATCATTTCA |
| | | TAATTTTACCCTCTGTTATATAAAAGAGACAGAAAAAGATTGC |
| | | CTCAATCTGGATAAAAACCTGATCAAATATGATTTGCAAAATT |
| | | TAAAACCTTATACGAAATATGTTTTATCATTACATGCCTACAT |
| | | CATTGCAAAAGTGCAACGTAATGGAAGTGCTGCAATGTGTCA |
| | | TTTCACAACTAAAAGTGCTCCTCCAAGCCAGGTCTGGAACATG |
| | | ACTGTCTCCATGACATCAGATAATAGTATGCATGTCAAGTGTA |
| | | GGCCTCCCAGGGACCGTAATGGCCCCCATGAACGTTACCATTT |
| | | GGAAGTTGAAGCTGGAAATACTCTGGTTAGAAATGAGTCGCA |
| | | TAAGAATTGCGATTTCCGTGTAAAAGATCTTCAATATTCAACA |
| | | GACTACACTTTTAAGGCCTATTTTCACAATGGAGACTATCCTG |
| | | GAGAACCCTTTATTTTACATCATTCAACATCTTATAATTCTAA |
| | | GGCACTGATAGCATTTCTGGCATTTCTGATTATTGTGACATCA |
| | | ATAGCCCTGCTTGTTGTTCTCTACAAAATCTATGATCTATAA |
| 40 | tCD45_RO_K277, | MTMYLWLKLLAFGFAFLDTEVFVTGEQKLISEEDLQSPTPSPTDA |
| | N278, S280 AA | YLNASETTTLSPSGSAVISTTTIATTPSKPTCDEKYANITVDYLYN |
| | | KETKLFTAKLNVNENVECGNNTCTNNEVHNLTECAAAAVSISHN |
| | | SCTAPDKTLILDVPPGVEKFQLHDCTQVEKADTTICLKWKNIETF |
| | | TCDTQNITYRFQCGNMIFDNKEIKLENLEPEHEYKCDSEILYNNH |
| | | KFTNASKIIKTDFGSPGEPQIIFCRSEAAHQGVITWNPPQRSFHNFT |
| | | LCYIKETEKDCLNLDKNLIKYDLQNLKPYTKYVLSLHAYIIAKVQ |
| | | RNGSAAMCHFTTKSAPPSQVWNMTVSMTSDNSMHVKCRPPRD |
| | | RNGPHERYHLEVEAGNTLVRNESHKNCDFRVKDLQYSTDYTFK |
| | | AYFHNGDYPGEPFILHHSTSYNSKALIAFLAFLIIVTSIALLVVLYK |
| | | IYDL* |
| 41 | tCD45_RO_I283, | ATGACCATGTATTTGTGGCTTAAACTCTTGGCATTTGGCTTTG |
| | H285, N286 | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGAAAAATATGCAAACATCACT |
| | | GTGGATTACTTATATAACAAGGAAACTAAATTATTTACAGCA |
| | | AAGCTAAATGTTAATGAGAATGTGGAATGTGGAAACAATACT |
| | | TGCACAAACAATGAGGTGCATAACCTTACAGAATGTAAAAAT |
| | | GCGTCTGTTTCCGCGTCTGCCGCTTCATGTACTGCTCCTGATA |
| | | AGACATTAATATTAGATGTGCCACCAGGGGTTGAAAAGTTTC |
| | | AGTTACATGATTGTACACAAGTTGAAAAAGCAGATACTACTA |
| | | TTTGTTTAAAATGGAAAAATATTGAAACCTTTACTTGTGATAC |
| | | ACAGAATATTACCTACAGATTTCAGTGTGGTAATATGATATTT |
| | | GATAATAAAGAAATTAAATTAGAAAACCTTGAACCCGAACAT |
| | | GAGTATAAGTGTGACTCAGAAATACTCTATAATAACCACAAG |
| | | TTTACTAACGCAAGTAAAATTATTAAAACAGATTTTGGGAGTC |
| | | CAGGAGAGCCTCAGATTATTTTTTGTAGAAGTGAAGCTGCAC |
| | | ATCAAGGAGTAATTACCTGGAATCCCCCTCAAAGATCATTTCA |
| | | TAATTTTACCCTCTGTTATATAAAAGAGACAGAAAAAGATTGC |
| | | CTCAATCTGGATAAAAACCTGATCAAATATGATTTGCAAAATT |
| | | TAAAACCTTATACGAAATATGTTTTATCATTACATGCCTACAT |
| | | CATTGCAAAAGTGCAACGTAATGGAAGTGCTGCAATGTGTCA |
| | | TTTCACAACTAAAAGTGCTCCTCCAAGCCAGGTCTGGAACATG |
| | | ACTGTCTCCATGACATCAGATAATAGTATGCATGTCAAGTGTA |
| | | GGCCTCCCAGGGACCGTAATGGCCCCCATGAACGTTACCATTT |
| | | GGAAGTTGAAGCTGGAAATACTCTGGTTAGAAATGAGTCGCA |
| | | TAAGAATTGCGATTTCCGTGTAAAAGATCTTCAATATTCAACA |
| | | GACTACACTTTTAAGGCCTATTTTCACAATGGAGACTATCCTG |
| | | GAGAACCCTTTATTTTACATCATTCAACATCTTATAATTCTAA |
| | | GGCACTGATAGCATTTCTGGCATTTCTGATTATTGTGACATCA |
| | | ATAGCCCTGCTTGTTGTTCTCTACAAAATCTATGATCTATAA |
| 42 | tCD45_RO_I283, | MTMYLWLKLLAFGFAFLDTEVFVTGEQKLISEEDLQSPTPSPTDA |
| | H285, N286 AA | YLNASETTTLSPSGSAVISTTTIATTPSKPTCDEKYANITVDYLYN |
| | | KETKLFTAKLNVNENVECGNNTCTNNEVHNLTECKNASVSASA |
| | | ASCTAPDKTLILDVPPGVEKFQLHDCTQVEKADTTICLKWKNIET |
| | | FTCDTQNITYRFQCGNMIFDNKEIKLENLEPEHEYKCDSEILYNN |
| | | HKFTNASKIIKTDFGSPGEPQIIFCRSEAAHQGVITWNPPQRSFHN |
| | | FTLCYIKETEKDCLNLDKNLIKYDLQNLKPYTKYVLSLHAYIIAK |
| | | VQRNGSAAMCHFTTKSAPPSQVWNMTVSMTSDNSMHVKCRPP |
| | | RDRNGPHERYHLEVEAGNTLVRNESHKNCDFRVKDLQYSTDYT |
| | | FKAYFHNGDYPGEPFILHHSTSYNSKALIAFLAFLIIVTSIALLVVL |
| | | YKIYDL* |
| 43 | tCD45_RO_E269- | ATGACCATGTATTTGTGGCTTAAACTCTTGGCATTTGGCTTTG |
| | H271 | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGAAAAATATGCAAACATCACT |
| | | GTGGATTACTTATATAACAAGGAAACTAAATTATTTACAGCA |
| | | AAGCTAAATGTTAATGAGAATGTGGAATGTGGAAACAATACT |
| | | TGCACAAACAATGCGGCCGCTAACCTTACAGAATGTAAAAAT |
| | | GCGTCTGTTTCCATATCTCATAATTCATGTACTGCTCCTGATAA |
| | | GACATTAATATTAGATGTGCCACCAGGGGTTGAAAAGTTTCA |
| | | GTTACATGATTGTACACAAGTTGAAAAAGCAGATACTACTATT |
| | | TGTTTAAAATGGAAAAATATTGAAACCTTTACTTGTGATACAC |
| | | AGAATATTACCTACAGATTTCAGTGTGGTAATATGATATTTGA |
| | | TAATAAAGAAATTAAATTAGAAAACCTTGAACCCGAACATGA |
| | | GTATAAGTGTGACTCAGAAATACTCTATAATAACCACAAGTTT |
| | | ACTAACGCAAGTAAAATTATTAAAACAGATTTTGGGAGTCCA |
| | | GGAGAGCCTCAGATTATTTTTTGTAGAAGTGAAGCTGCACATC |
| | | AAGGAGTAATTACCTGGAATCCCCCTCAAAGATCATTTCATAA |
| | | TTTTACCCTCTGTTATATAAAAGAGACAGAAAAAGATTGCCTC |
| | | AATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 44 | tCD45_RO_E269- | MTMYLWLKLLAFGFAFLDTEVFVTGEQKLISEEDLQSPTPSPTDA |
| | H271 AA | YLNASETTTLSPSGSAVISTTTIATTPSKPTCDEKYANITVDYLYN |
| | | KETKLFTAKLNVNENVECGNNTCTNNAAANLTECKNASVSISHN |
| | | SCTAPDKTLILDVPPGVEKFQLHDCTQVEKADTTICLKWKNIETF |
| | | TCDTQNITYRFQCGNMIFDNKEIKLENLEPEHEYKCDSEILYNNH |
| | | KFTNASKIIKTDFGSPGEPQIIFCRSEAAHQGVITWNPPQRSFHNFT |
| | | LCYIKETEKDCLNLDKNLIKYDLQNLKPYTKYVLSLHAYIIAKVQ |
| | | RNGSAAMCHFTTKSAPPSQVWNMTVSMTSDNSMHVKCRPPRD |
| | | RNGPHERYHLEVEAGNTLVRNESHKNCDFRVKDLQYSTDYTFK |
| | | AYFHNGDYPGEPFILHHSTSYNSKALIAFLAFLIIVTSIALLVVLYK |
| | | IYDL* |
| 45 | tCD45RO_D238, | ATGACCATGTATTTGTGGCTTAAACTCTTGGCATTTGGCTTTG |
| | L240 | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGAAAAATATGCAAACATCACT |
| | | GTGGCCTACGCGTATAACAAGGAAACTAAATTATTTACAGCA |
| | | AAGCTAAATGTTAATGAGAATGTGGAATGTGGAAACAATACT |
| | | TGCACAAACAATGAGGTGCATAACCTTACAGAATGTAAAAAT |
| | | GCGTCTGTTTCCATATCTCATAATTCATGTACTGCTCCTGATAA |
| | | GACATTAATATTAGATGTGCCACCAGGGGTTGAAAAGTTTCA |
| | | GTTACATGATTGTACACAAGTTGAAAAAGCAGATACTACTATT |
| | | TGTTTAAAATGGAAAAATATTGAAACCTTTACTTGTGATACAC |
| | | AGAATATTACCTACAGATTTCAGTGTGGTAATATGATATTTGA |
| | | TAATAAAGAAATTAAATTAGAAAACCTTGAACCCGAACATGA |
| | | GTATAAGTGTGACTCAGAAATACTCTATAATAACCACAAGTTT |
| | | ACTAACGCAAGTAAAATTATTAAAACAGATTTTGGGAGTCCA |
| | | GGAGAGCCTCAGATTATTTTTTGTAGAAGTGAAGCTGCACATC |
| | | AAGGAGTAATTACCTGGAATCCCCCTCAAAGATCATTTCATAA |
| | | TTTTACCCTCTGTTATATAAAAGAGACAGAAAAAGATTGCCTC |
| | | AATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 46 | tCD45RO_D238, | MTMYLWLKLLAFGFAFLDTEVFVTGEQKLISEEDLQSPTPSPTDA |
| | L240 AA | YLNASETTTLSPSGSAVISTTTIATTPSKPTCDEKYANITVAYAYN |
| | | KETKLFTAKLNVNENVECGNNTCTNNEVHNLTECKNASVSISHN |
| | | SCTAPDKTLILDVPPGVEKFQLHDCTQVEKADTTICLKWKNIETF |
| | | TCDTQNITYRFQCGNMIFDNKEIKLENLEPEHEYKCDSEILYNNH |
| | | KFTNASKIIKTDFGSPGEPQIIFCRSEAAHQGVITWNPPQRSFHNFT |
| | | LCYIKETEKDCLNLDKNLIKYDLQNLKPYTKYVLSLHAYIIAKVQ |
| | | RNGSAAMCHFTTKSAPPSQVWNMTVSMTSDNSMHVKCRPPRD |
| | | RNGPHERYHLEVEAGNTLVRNESHKNCDFRVKDLQYSTDYTFK |
| | | AYFHNGDYPGEPFILHHSTSYNSKALIAFLAFLIIVTSIALLVVLYK |
| | | IYDL* |
| 47 | tCD45RO_Y232A | ATGACCATGTATTTGTGGCTTAAACTCTTGGCATTTGGCTTTG |
| | | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGAAAAAGCCGCAAACATCACT |
| | | GTGGATTACTTATATAACAAGGAAACTAAATTATTTACAGCA |
| | | AAGCTAAATGTTAATGAGAATGTGGAATGTGGAAACAATACT |
| | | TGCACAAACAATGAGGTGCATAACCTTACAGAATGTAAAAAT |
| | | GCGTCTGTTTCCATATCTCATAATTCATGTACTGCTCCTGATAA |
| | | GACATTAATATTAGATGTGCCACCAGGGGTTGAAAAGTTTCA |
| | | GTTACATGATTGTACACAAGTTGAAAAAGCAGATACTACTATT |
| | | TGTTTAAAATGGAAAAATATTGAAACCTTTACTTGTGATACAC |
| | | AGAATATTACCTACAGATTTCAGTGTGGTAATATGATATTTGA |
| | | TAATAAAGAAATTAAATTAGAAAACCTTGAACCCGAACATGA |
| | | GTATAAGTGTGACTCAGAAATACTCTATAATAACCACAAGTTT |
| | | ACTAACGCAAGTAAAATTATTAAAACAGATTTTGGGAGTCCA |
| | | GGAGAGCCTCAGATTATTTTTTGTAGAAGTGAAGCTGCACATC |
| | | AAGGAGTAATTACCTGGAATCCCCCTCAAAGATCATTTCATAA |
| | | TTTTACCCTCTGTTATATAAAAGAGACAGAAAAAGATTGCCTC |
| | | AATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 48 | tCD45RO_Y232A | MTMYLWLKLLAFGFAFLDTEVFVTGEQKLISEEDLQSPTPSPTDA |
| | AA | YLNASETTTLSPSGSAVISTTTIATTPSKPTCDEKAANITVDYLYN |
| | | KETKLFTAKLNVNENVECGNNTCTNNEVHNLTECKNASVSISHN |
| | | SCTAPDKTLILDVPPGVEKFQLHDCTQVEKADTTICLKWKNIETF |
| | | TCDTQNITYRFQCGNMIFDNKEIKLENLEPEHEYKCDSEILYNNH |
| | | KFTNASKIIKTDFGSPGEPQIIFCRSEAAHQGVITWNPPQRSFHNFT |
| | | LCYIKETEKDCLNLDKNLIKYDLQNLKPYTKYVLSLHAYIIAKVQ |
| | | RNGSAAMCHFTTKSAPPSQVWNMTVSMTSDNSMHVKCRPPRD |
| | | RNGPHERYHLEVEAGNTLVRNESHKNCDFRVKDLQYSTDYTFK |
| | | AYFHNGDYPGEPFILHHSTSYNSKALIAFLAFLIIVTSIALLVVLYK |
| | | IYDL* |
| 49 | tCD45RO_V303- | ATGACCATGTATTTGTGGCTTAAACTCTTGGCATTTGGCTTTG |
| | Q307 | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGAAAAATATGCAAACATCACT |
| | | GTGGATTACTTATATAACAAGGAAACTAAATTATTTACAGCA |
| | | AAGCTAAATGTTAATGAGAATGTGGAATGTGGAAACAATACT |
| | | TGCACAAACAATGAGGTGCATAACCTTACAGAATGTAAAAAT |
| | | GCGTCTGTTTCCATATCTCATAATTCATGTACTGCTCCTGATAA |
| | | GACATTAATATTAGATGTGCCACCAGGGGCCGCAGCTGCTGC |
| | | GTTACATGATTGTACACAAGTTGAAAAAGCAGATACTACTATT |
| | | TGTTTAAAATGGAAAAATATTGAAACCTTTACTTGTGATACAC |
| | | AGAATATTACCTACAGATTTCAGTGTGGTAATATGATATTTGA |
| | | TAATAAAGAAATTAAATTAGAAAACCTTGAACCCGAACATGA |
| | | GTATAAGTGTGACTCAGAAATACTCTATAATAACCACAAGTTT |
| | | ACTAACGCAAGTAAAATTATTAAAACAGATTTTGGGAGTCCA |
| | | GGAGAGCCTCAGATTATTTTTTGTAGAAGTGAAGCTGCACATC |
| | | AAGGAGTAATTACCTGGAATCCCCCTCAAAGATCATTTCATAA |
| | | TTTTACCCTCTGTTATATAAAAGAGACAGAAAAAGATTGCCTC |
| | | AATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 50 | tCD45RO_V303- | MTMYLWLKLLAFGFAFLDTEVFVTGEQKLISEEDLQSPTPSPTDA |
| | Q307 AA | YLNASETTTLSPSGSAVISTTTIATTPSKPTCDEKYANITVDYLYN |
| | | KETKLFTAKLNVNENVECGNNTCTNNEVHNLTECKNASVSISHN |
| | | SCTAPDKTLILDVPPGAAAAALHDCTQVEKADTTICLKWKNIETF |
| | | TCDTQNITYRFQCGNMIFDNKEIKLENLEPEHEYKCDSEILYNNH |
| | | KFTNASKIIKTDFGSPGEPQIIFCRSEAAHQGVITWNPPQRSFHNFT |
| | | LCYIKETEKDCLNLDKNLIKYDLQNLKPYTKYVLSLHAYIIAKVQ |
| | | RNGSAAMCHFTTKSAPPSQVWNMTVSMTSDNSMHVKCRPPRD |
| | | RNGPHERYHLEVEAGNTLVRNESHKNCDFRVKDLQYSTDYTFK |
| | | AYFHNGDYPGEPFILHHSTSYNSKALIAFLAFLIIVTSIALLVVLYK |
| | | IYDL* |
| 51 | tCD45RO_N374- | ATGACCATGTATTTGTGGCTTAAACTCTTGGCATTTGGCTTTG |
| | K377 | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGAAAAATATGCAAACATCACT |
| | | GTGGATTACTTATATAACAAGGAAACTAAATTATTTACAGCA |
| | | AAGCTAAATGTTAATGAGAATGTGGAATGTGGAAACAATACT |
| | | TGCACAAACAATGAGGTGCATAACCTTACAGAATGTAAAAAT |
| | | GCGTCTGTTTCCATATCTCATAATTCATGTACTGCTCCTGATAA |
| | | GACATTAATATTAGATGTGCCACCAGGGGTTGAAAAGTTTCA |
| | | GTTACATGATTGTACACAAGTTGAAAAAGCAGATACTACTATT |
| | | TGTTTAAAATGGAAAAATATTGAAACCTTTACTTGTGATACAC |
| | | AGAATATTACCTACAGATTTCAGTGTGGTAATATGATATTTGA |
| | | TAATAAAGAAATTAAATTAGAAAACCTTGAACCCGAACATGA |
| | | GTATAAGTGTGACTCAGAAATACTCTATGCCGCTGCAGCCTTT |
| | | ACTAACGCAAGTAAAATTATTAAAACAGATTTTGGGAGTCCA |
| | | GGAGAGCCTCAGATTATTTTTTGTAGAAGTGAAGCTGCACATC |
| | | AAGGAGTAATTACCTGGAATCCCCCTCAAAGATCATTTCATAA |
| | | TTTTACCCTCTGTTATATAAAAGAGACAGAAAAAGATTGCCTC |
| | | AATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 52 | tCD45RO_N374- | MTMYLWLKLLAFGFAFLDTEVFVTGEQKLISEEDLQSPTPSPTDA |
| | K377 AA | YLNASETTTLSPSGSAVISTTTIATTPSKPTCDEKYANITVDYLYN |
| | | KETKLFTAKLNVNENVECGNNTCTNNEVHNLTECKNASVSISHN |
| | | SCTAPDKTLILDVPPGVEKFQLHDCTQVEKADTTICLKWKNIETF |
| | | TCDTQNITYRFQCGNMIFDNKEIKLENLEPEHEYKCDSEILYAAA |
| | | AFTNASKIIKTDFGSPGEPQIIFCRSEAAHQGVITWNPPQRSFHNFT |
| | | LCYIKETEKDCLNLDKNLIKYDLQNLKPYTKYVLSLHAYIIAKVQ |
| | | RNGSAAMCHFTTKSAPPSQVWNMTVSMTSDNSMHVKCRPPRD |
| | | RNGPHERYHLEVEAGNTLVRNESHKNCDFRVKDLQYSTDYTFK |
| | | AYFHNGDYPGEPFILHHSTSYNSKALIAFLAFLIIVTSIALLVVLYK |
| | | IYDL* |
| 53 | tCD45RO_N286A | ATGACCATGTATTTGTGGCTTAAACTCTTGGCATTTGGCTTTG |
| | | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGAAAAATATGCAAACATCACT |
| | | GTGGATTACTTATATAACAAGGAAACTAAATTATTTACAGCA |
| | | AAGCTAAATGTTAATGAGAATGTGGAATGTGGAAACAATACT |
| | | TGCACAAACAATGAGGTGCATAACCTTACAGAATGTAAAAAT |
| | | GCGTCTGTTTCCATATCTCATGCTTCATGTACTGCTCCTGATAA |
| | | GACATTAATATTAGATGTGCCACCAGGGGTTGAAAAGTTTCA |
| | | GTTACATGATTGTACACAAGTTGAAAAAGCAGATACTACTATT |
| | | TGTTTAAAATGGAAAAATATTGAAACCTTTACTTGTGATACAC |
| | | AGAATATTACCTACAGATTTCAGTGTGGTAATATGATATTTGA |
| | | TAATAAAGAAATTAAATTAGAAAACCTTGAACCCGAACATGA |
| | | GTATAAGTGTGACTCAGAAATACTCTATAATAACCACAAGTTT |
| | | ACTAACGCAAGTAAAATTATTAAAACAGATTTTGGGAGTCCA |
| | | GGAGAGCCTCAGATTATTTTTTGTAGAAGTGAAGCTGCACATC |
| | | AAGGAGTAATTACCTGGAATCCCCCTCAAAGATCATTTCATAA |
| | | TTTTACCCTCTGTTATATAAAAGAGACAGAAAAAGATTGCCTC |
| | | AATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 54 | tCD45RO_N286A | MTMYLWLKLLAFGFAFLDTEVFVTGEQKLISEEDLQSPTPSPTDA |
| | AA | YLNASETTTLSPSGSAVISTTTIATTPSKPTCDEKYANITVDYLYN |
| | | KETKLFTAKLNVNENVECGNNTCTNNEVHNLTECKNASVSISHA |
| | | SCTAPDKTLILDVPPGVEKFQLHDCTQVEKADTTICLKWKNIETF |
| | | TCDTQNITYRFQCGNMIFDNKEIKLENLEPEHEYKCDSEILYNNH |
| | | KFTNASKIIKTDFGSPGEPQIIFCRSEAAHQGVITWNPPQRSFHNFT |
| | | LCYIKETEKDCLNLDKNLIKYDLQNLKPYTKYVLSLHAYIIAKVQ |
| | | RNGSAAMCHFTTKSAPPSQVWNMTVSMTSDNSMHVKCRPPRD |
| | | RNGPHERYHLEVEAGNTLVRNESHKNCDFRVKDLQYSTDYTFK |
| | | AYFHNGDYPGEPFILHHSTSYNSKALIAFLAFLIIVTSIALLVVLYK |
| | | IYDL* |
| 55 | tCD45RO_K231A, | ATGACCATGTATTTGTGGCTTAAACTCTTGGCATTTGGCTTTG |
| | Y232A | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGAAGCCGCTGCAAACATCACT |
| | | GTGGATTACTTATATAACAAGGAAACTAAATTATTTACAGCA |
| | | AAGCTAAATGTTAATGAGAATGTGGAATGTGGAAACAATACT |
| | | TGCACAAACAATGAGGTGCATAACCTTACAGAATGTAAAAAT |
| | | GCGTCTGTTTCCATATCTCATAATTCATGTACTGCTCCTGATAA |
| | | GACATTAATATTAGATGTGCCACCAGGGGTTGAAAAGTTTCA |
| | | GTTACATGATTGTACACAAGTTGAAAAAGCAGATACTACTATT |
| | | TGTTTAAAATGGAAAAATATTGAAACCTTTACTTGTGATACAC |
| | | AGAATATTACCTACAGATTTCAGTGTGGTAATATGATATTTGA |
| | | TAATAAAGAAATTAAATTAGAAAACCTTGAACCCGAACATGA |
| | | GTATAAGTGTGACTCAGAAATACTCTATAATAACCACAAGTTT |
| | | ACTAACGCAAGTAAAATTATTAAAACAGATTTTGGGAGTCCA |
| | | GGAGAGCCTCAGATTATTTTTTGTAGAAGTGAAGCTGCACATC |
| | | AAGGAGTAATTACCTGGAATCCCCCTCAAAGATCATTTCATAA |
| | | TTTTACCCTCTGTTATATAAAAGAGACAGAAAAAGATTGCCTC |
| | | AATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 56 | tCD45RO_K231A, | MTMYLWLKLLAFGFAFLDTEVFVTGEQKLISEEDLQSPTPSPTDA |
| | Y232A AA | YLNASETTTLSPSGSAVISTTTIATTPSKPTCDEAAANITVDYLYN |
| | | KETKLFTAKLNVNENVECGNNTCTNNEVHNLTECKNASVSISHN |
| | | SCTAPDKTLILDVPPGVEKFQLHDCTQVEKADTTICLKWKNIETF |
| | | TCDTQNITYRFQCGNMIFDNKEIKLENLEPEHEYKCDSEILYNNH |
| | | KFTNASKIIKTDFGSPGEPQIIFCRSEAAHQGVITWNPPQRSFHNFT |
| | | LCYIKETEKDCLNLDKNLIKYDLQNLKPYTKYVLSLHAYIIAKVQ |
| | | RNGSAAMCHFTTKSAPPSQVWNMTVSMTSDNSMHVKCRPPRD |
| | | RNGPHERYHLEVEAGNTLVRNESHKNCDFRVKDLQYSTDYTFK |
| | | AYFHNGDYPGEPFILHHSTSYNSKALIAFLAFLIIVTSIALLVVLYK |
| | | IYDL* |
| 57 | tCD45RO_K231A | ATGACCATGTATTTGTGGCTTAAACTCTTGGCATTTGGCTTTG |
| | | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGAAGCATATGCAAACATCACT |
| | | GTGGATTACTTATATAACAAGGAAACTAAATTATTTACAGCA |
| | | AAGCTAAATGTTAATGAGAATGTGGAATGTGGAAACAATACT |
| | | TGCACAAACAATGAGGTGCATAACCTTACAGAATGTAAAAAT |
| | | GCGTCTGTTTCCATATCTCATAATTCATGTACTGCTCCTGATAA |
| | | GACATTAATATTAGATGTGCCACCAGGGGTTGAAAAGTTTCA |
| | | GTTACATGATTGTACACAAGTTGAAAAAGCAGATACTACTATT |
| | | TGTTTAAAATGGAAAAATATTGAAACCTTTACTTGTGATACAC |
| | | AGAATATTACCTACAGATTTCAGTGTGGTAATATGATATTTGA |
| | | TAATAAAGAAATTAAATTAGAAAACCTTGAACCCGAACATGA |
| | | GTATAAGTGTGACTCAGAAATACTCTATAATAACCACAAGTTT |
| | | ACTAACGCAAGTAAAATTATTAAAACAGATTTTGGGAGTCCA |
| | | GGAGAGCCTCAGATTATTTTTTGTAGAAGTGAAGCTGCACATC |
| | | AAGGAGTAATTACCTGGAATCCCCCTCAAAGATCATTTCATAA |
| | | TTTTACCCTCTGTTATATAAAAGAGACAGAAAAAGATTGCCTC |
| | | AATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 58 | tCD45RO_K231A | MTMYLWLKLLAFGFAFLDTEVFVTGEQKLISEEDLQSPTPSPTDA |
| | AA | YLNASETTTLSPSGSAVISTTTIATTPSKPTCDEAYANITVDYLYN |
| | | KETKLFTAKLNVNENVECGNNTCTNNEVHNLTECKNASVSISHN |
| | | SCTAPDKTLILDVPPGVEKFQLHDCTQVEKADTTICLKWKNIETF |
| | | TCDTQNITYRFQCGNMIFDNKEIKLENLEPEHEYKCDSEILYNNH |
| | | KFTNASKIIKTDFGSPGEPQIIFCRSEAAHQGVITWNPPQRSFHNFT |
| | | LCYIKETEKDCLNLDKNLIKYDLQNLKPYTKYVLSLHAYIIAKVQ |
| | | RNGSAAMCHFTTKSAPPSQVWNMTVSMTSDNSMHVKCRPPRD |
| | | RNGPHERYHLEVEAGNTLVRNESHKNCDFRVKDLQYSTDYTFK |
| | | AYFHNGDYPGEPFILHHSTSYNSKALIAFLAFLIIVTSIALLVVLYK |
| | | IYDL* |
| 59 | tCD45RO_I328- | ATGACCATGTATTTGTGGCTTAAACTCTTGGCATTTGGCTTTG |
| | T332 | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGAAAAATATGCAAACATCACT |
| | | GTGGATTACTTATATAACAAGGAAACTAAATTATTTACAGCA |
| | | AAGCTAAATGTTAATGAGAATGTGGAATGTGGAAACAATACT |
| | | TGCACAAACAATGAGGTGCATAACCTTACAGAATGTAAAAAT |
| | | GCGTCTGTTTCCATATCTCATAATTCATGTACTGCTCCTGATAA |
| | | GACATTAATATTAGATGTGCCACCAGGGGTTGAAAAGTTTCA |
| | | GTTACATGATTGTACACAAGTTGAAAAAGCAGATACTACTATT |
| | | TGTTTAAAATGGAAAAATGCAGCCGCCGCTGCCTGTGATACA |
| | | CAGAATATTACCTACAGATTTCAGTGTGGTAATATGATATTTG |
| | | ATAATAAAGAAATTAAATTAGAAAACCTTGAACCCGAACATG |
| | | AGTATAAGTGTGACTCAGAAATACTCTATAATAACCACAAGT |
| | | TTACTAACGCAAGTAAAATTATTAAAACAGATTTTGGGAGTCC |
| | | AGGAGAGCCTCAGATTATTTTTTGTAGAAGTGAAGCTGCACAT |
| | | CAAGGAGTAATTACCTGGAATCCCCCTCAAAGATCATTTCATA |
| | | ATTTTACCCTCTGTTATATAAAAGAGACAGAAAAAGATTGCCT |
| | | CAATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 60 | tCD45RO_I328- | MTMYLWLKLLAFGFAFLDTEVFVTGEQKLISEEDLQSPTPSPTDA |
| | T332 AA | YLNASETTTLSPSGSAVISTTTIATTPSKPTCDEKYANITVDYLYN |
| | | KETKLFTAKLNVNENVECGNNTCTNNEVHNLTECKNASVSISHN |
| | | SCTAPDKTLILDVPPGVEKFQLHDCTQVEKADTTICLKWKNAAA |
| | | AACDTQNITYRFQCGNMIFDNKEIKLENLEPEHEYKCDSEILYNN |
| | | HKFTNASKIIKTDFGSPGEPQIIFCRSEAAHQGVITWNPPQRSFHN |
| | | FTLCYIKETEKDCLNLDKNLIKYDLQNLKPYTKYVLSLHAYIIAK |
| | | VQRNGSAAMCHFTTKSAPPSQVWNMTVSMTSDNSMHVKCRPP |
| | | RDRNGPHERYHLEVEAGNTLVRNESHKNCDFRVKDLQYSTDYT |
| | | FKAYFHNGDYPGEPFILHHSTSYNSKALIAFLAFLIIVTSIALLVVL |
| | | YKIYDL* |
| 61 | tCD45RO_I283, | ATGACCATGTATTTGTGGCTTAAACTCTTGGCATTTGGCTTTG |
| | N286 | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGAAAAATATGCAAACATCACT |
| | | GTGGATTACTTATATAACAAGGAAACTAAATTATTTACAGCA |
| | | AAGCTAAATGTTAATGAGAATGTGGAATGTGGAAACAATACT |
| | | TGCACAAACAATGAGGTGCATAACCTTACAGAATGTAAAAAT |
| | | GCGTCTGTTTCCGCCTCTCATGCTTCATGTACTGCTCCTGATAA |
| | | GACATTAATATTAGATGTGCCACCAGGGGTTGAAAAGTTTCA |
| | | GTTACATGATTGTACACAAGTTGAAAAAGCAGATACTACTATT |
| | | TGTTTAAAATGGAAAAATATTGAAACCTTTACTTGTGATACAC |
| | | AGAATATTACCTACAGATTTCAGTGTGGTAATATGATATTTGA |
| | | TAATAAAGAAATTAAATTAGAAAACCTTGAACCCGAACATGA |
| | | GTATAAGTGTGACTCAGAAATACTCTATAATAACCACAAGTTT |
| | | ACTAACGCAAGTAAAATTATTAAAACAGATTTTGGGAGTCCA |
| | | GGAGAGCCTCAGATTATTTTTTGTAGAAGTGAAGCTGCACATC |
| | | AAGGAGTAATTACCTGGAATCCCCCTCAAAGATCATTTCATAA |
| | | TTTTACCCTCTGTTATATAAAAGAGACAGAAAAAGATTGCCTC |
| | | AATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 62 | tCD45RO_I283, | MTMYLWLKLLAFGFAFLDTEVFVTGEQKLISEEDLQSPTPSPTDA |
| | N286 AA | YLNASETTTLSPSGSAVISTTTIATTPSKPTCDEKYANITVDYLYN |
| | | KETKLFTAKLNVNENVECGNNTCTNNEVHNLTECKNASVSASH |
| | | ASCTAPDKTLILDVPPGVEKFQLHDCTQVEKADTTICLKWKNIET |
| | | FTCDTQNITYRFQCGNMIFDNKEIKLENLEPEHEYKCDSEILYNN |
| | | HKFTNASKIIKTDFGSPGEPQIIFCRSEAAHQGVITWNPPQRSFHN |
| | | FTLCYIKETEKDCLNLDKNLIKYDLQNLKPYTKYVLSLHAYIIAK |
| | | VQRNGSAAMCHFTTKSAPPSQVWNMTVSMTSDNSMHVKCRPP |
| | | RDRNGPHERYHLEVEAGNTLVRNESHKNCDFRVKDLQYSTDYT |
| | | FKAYFHNGDYPGEPFILHHSTSYNSKALIAFLAFLIIVTSIALLVVL |
| | | YKIYDL* |
| 63 | tCD45RO_I283A, | ATGACCATGTATTTGTGGCTTAAACTCTTGGCATTTGGCTTTG |
| | H285A | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGAAAAATATGCAAACATCACT |
| | | GTGGATTACTTATATAACAAGGAAACTAAATTATTTACAGCA |
| | | AAGCTAAATGTTAATGAGAATGTGGAATGTGGAAACAATACT |
| | | TGCACAAACAATGAGGTGCATAACCTTACAGAATGTAAAAAT |
| | | GCGTCTGTTTCCGCCTCTGCTAATTCATGTACTGCTCCTGATAA |
| | | GACATTAATATTAGATGTGCCACCAGGGGTTGAAAAGTTTCA |
| | | GTTACATGATTGTACACAAGTTGAAAAAGCAGATACTACTATT |
| | | TGTTTAAAATGGAAAAATATTGAAACCTTTACTTGTGATACAC |
| | | AGAATATTACCTACAGATTTCAGTGTGGTAATATGATATTTGA |
| | | TAATAAAGAAATTAAATTAGAAAACCTTGAACCCGAACATGA |
| | | GTATAAGTGTGACTCAGAAATACTCTATAATAACCACAAGTTT |
| | | ACTAACGCAAGTAAAATTATTAAAACAGATTTTGGGAGTCCA |
| | | GGAGAGCCTCAGATTATTTTTTGTAGAAGTGAAGCTGCACATC |
| | | AAGGAGTAATTACCTGGAATCCCCCTCAAAGATCATTTCATAA |
| | | TTTTACCCTCTGTTATATAAAAGAGACAGAAAAAGATTGCCTC |
| | | AATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 64 | tCD45RO_I283A, | MTMYLWLKLLAFGFAFLDTEVFVTGEQKLISEEDLQSPTPSPTDA |
| | H285A AA | YLNASETTTLSPSGSAVISTTTIATTPSKPTCDEKYANITVDYLYN |
| | | KETKLFTAKLNVNENVECGNNTCTNNEVHNLTECKNASVSASA |
| | | NSCTAPDKTLILDVPPGVEKFQLHDCTQVEKADTTICLKWKNIET |
| | | FTCDTQNITYRFQCGNMIFDNKEIKLENLEPEHEYKCDSEILYNN |
| | | HKFTNASKIIKTDFGSPGEPQIIFCRSEAAHQGVITWNPPQRSFHN |
| | | FTLCYIKETEKDCLNLDKNLIKYDLQNLKPYTKYVLSLHAYIIAK |
| | | VQRNGSAAMCHFTTKSAPPSQVWNMTVSMTSDNSMHVKCRPP |
| | | RDRNGPHERYHLEVEAGNTLVRNESHKNCDFRVKDLQYSTDYT |
| | | FKAYFHNGDYPGEPFILHHSTSYNSKALIAFLAFLIIVTSIALLVVL |
| | | YKIYDL* |
| 65 | tCD45RO_I283A | ATGACCATGTATTTGTGGCTTAAACTCTTGGCATTTGGCTTTG |
| | | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGAAAAATATGCAAACATCACT |
| | | GTGGATTACTTATATAACAAGGAAACTAAATTATTTACAGCA |
| | | AAGCTAAATGTTAATGAGAATGTGGAATGTGGAAACAATACT |
| | | TGCACAAACAATGAGGTGCATAACCTTACAGAATGTAAAAAT |
| | | GCGTCTGTTTCCGCCTCTCATAATTCATGTACTGCTCCTGATAA |
| | | GACATTAATATTAGATGTGCCACCAGGGGTTGAAAAGTTTCA |
| | | GTTACATGATTGTACACAAGTTGAAAAAGCAGATACTACTATT |
| | | TGTTTAAAATGGAAAAATATTGAAACCTTTACTTGTGATACAC |
| | | AGAATATTACCTACAGATTTCAGTGTGGTAATATGATATTTGA |
| | | TAATAAAGAAATTAAATTAGAAAACCTTGAACCCGAACATGA |
| | | GTATAAGTGTGACTCAGAAATACTCTATAATAACCACAAGTTT |
| | | ACTAACGCAAGTAAAATTATTAAAACAGATTTTGGGAGTCCA |
| | | GGAGAGCCTCAGATTATTTTTTGTAGAAGTGAAGCTGCACATC |
| | | AAGGAGTAATTACCTGGAATCCCCCTCAAAGATCATTTCATAA |
| | | TTTTACCCTCTGTTATATAAAAGAGACAGAAAAAGATTGCCTC |
| | | AATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 66 | tCD45RO_I283A | MTMYLWLKLLAFGFAFLDTEVFVTGEQKLISEEDLQSPTPSPTDA |
| | AA | YLNASETTTLSPSGSAVISTTTIATTPSKPTCDEKYANITVDYLYN |
| | | KETKLFTAKLNVNENVECGNNTCTNNEVHNLTECKNASVSASH |
| | | NSCTAPDKTLILDVPPGVEKFQLHDCTQVEKADTTICLKWKNIET |
| | | FTCDTQNITYRFQCGNMIFDNKEIKLENLEPEHEYKCDSEILYNN |
| | | HKFTNASKIIKTDFGSPGEPQIIFCRSEAAHQGVITWNPPQRSFHN |
| | | FTLCYIKETEKDCLNLDKNLIKYDLQNLKPYTKYVLSLHAYIIAK |
| | | VQRNGSAAMCHFTTKSAPPSQVWNMTVSMTSDNSMHVKCRPP |
| | | RDRNGPHERYHLEVEAGNTLVRNESHKNCDFRVKDLQYSTDYT |
| | | FKAYFHNGDYPGEPFILHHSTSYNSKALIAFLAFLIIVTSIALLVVL |
| | | YKIYDL* |
| 67 | tCD45RO_H285A, | ATGACCATGTATTTGTGGCTTAAACTCTTGGCATTTGGCTTTG |
| | N286A | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGAAAAATATGCAAACATCACT |
| | | GTGGATTACTTATATAACAAGGAAACTAAATTATTTACAGCA |
| | | AAGCTAAATGTTAATGAGAATGTGGAATGTGGAAACAATACT |
| | | TGCACAAACAATGAGGTGCATAACCTTACAGAATGTAAAAAT |
| | | GCGTCTGTTTCCATATCTGCTGCTTCATGTACTGCTCCTGATAA |
| | | GACATTAATATTAGATGTGCCACCAGGGGTTGAAAAGTTTCA |
| | | GTTACATGATTGTACACAAGTTGAAAAAGCAGATACTACTATT |
| | | TGTTTAAAATGGAAAAATATTGAAACCTTTACTTGTGATACAC |
| | | AGAATATTACCTACAGATTTCAGTGTGGTAATATGATATTTGA |
| | | TAATAAAGAAATTAAATTAGAAAACCTTGAACCCGAACATGA |
| | | GTATAAGTGTGACTCAGAAATACTCTATAATAACCACAAGTTT |
| | | ACTAACGCAAGTAAAATTATTAAAACAGATTTTGGGAGTCCA |
| | | GGAGAGCCTCAGATTATTTTTTGTAGAAGTGAAGCTGCACATC |
| | | AAGGAGTAATTACCTGGAATCCCCCTCAAAGATCATTTCATAA |
| | | TTTTACCCTCTGTTATATAAAAGAGACAGAAAAAGATTGCCTC |
| | | AATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 68 | tCD45RO_H285A, | MTMYLWLKLLAFGFAFLDTEVFVTGEQKLISEEDLQSPTPSPTDA |
| | N286A AA | YLNASETTTLSPSGSAVISTTTIATTPSKPTCDEKYANITVDYLYN |
| | | KETKLFTAKLNVNENVECGNNTCTNNEVHNLTECKNASVSISAA |
| | | SCTAPDKTLILDVPPGVEKFQLHDCTQVEKADTTICLKWKNIETF |
| | | TCDTQNITYRFQCGNMIFDNKEIKLENLEPEHEYKCDSEILYNNH |
| | | KFTNASKIIKTDFGSPGEPQIIFCRSEAAHQGVITWNPPQRSFHNFT |
| | | LCYIKETEKDCLNLDKNLIKYDLQNLKPYTKYVLSLHAYIIAKVQ |
| | | RNGSAAMCHFTTKSAPPSQVWNMTVSMTSDNSMHVKCRPPRD |
| | | RNGPHERYHLEVEAGNTLVRNESHKNCDFRVKDLQYSTDYTFK |
| | | AYFHNGDYPGEPFILHHSTSYNSKALIAFLAFLIIVTSIALLVVLYK |
| | | IYDL* |
| 69 | tCD45RO_H285A | ATGACCATGTATTTGTGGCTTAAACTCTTGGCATTTGGCTTTG |
| | | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGAAAAATATGCAAACATCACT |
| | | GTGGATTACTTATATAACAAGGAAACTAAATTATTTACAGCA |
| | | AAGCTAAATGTTAATGAGAATGTGGAATGTGGAAACAATACT |
| | | TGCACAAACAATGAGGTGCATAACCTTACAGAATGTAAAAAT |
| | | GCGTCTGTTTCCATATCTGCTAATTCATGTACTGCTCCTGATAA |
| | | GACATTAATATTAGATGTGCCACCAGGGGTTGAAAAGTTTCA |
| | | GTTACATGATTGTACACAAGTTGAAAAAGCAGATACTACTATT |
| | | TGTTTAAAATGGAAAAATATTGAAACCTTTACTTGTGATACAC |
| | | AGAATATTACCTACAGATTTCAGTGTGGTAATATGATATTTGA |
| | | TAATAAAGAAATTAAATTAGAAAACCTTGAACCCGAACATGA |
| | | GTATAAGTGTGACTCAGAAATACTCTATAATAACCACAAGTTT |
| | | ACTAACGCAAGTAAAATTATTAAAACAGATTTTGGGAGTCCA |
| | | GGAGAGCCTCAGATTATTTTTTGTAGAAGTGAAGCTGCACATC |
| | | AAGGAGTAATTACCTGGAATCCCCCTCAAAGATCATTTCATAA |
| | | TTTTACCCTCTGTTATATAAAAGAGACAGAAAAAGATTGCCTC |
| | | AATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 70 | tCD45RO_H285A | MTMYLWLKLLAFGFAFLDTEVFVTGEQKLISEEDLQSPTPSPTDA |
| | AA | YLNASETTTLSPSGSAVISTTTIATTPSKPTCDEKYANITVDYLYN |
| | | KETKLFTAKLNVNENVECGNNTCTNNEVHNLTECKNASVSISAN |
| | | SCTAPDKTLILDVPPGVEKFQLHDCTQVEKADTTICLKWKNIETF |
| | | TCDTQNITYRFQCGNMIFDNKEIKLENLEPEHEYKCDSEILYNNH |
| | | KFTNASKIIKTDFGSPGEPQIIFCRSEAAHQGVITWNPPQRSFHNFT |
| | | LCYIKETEKDCLNLDKNLIKYDLQNLKPYTKYVLSLHAYIIAKVQ |
| | | RNGSAAMCHFTTKSAPPSQVWNMTVSMTSDNSMHVKCRPPRD |
| | | RNGPHERYHLEVEAGNTLVRNESHKNCDFRVKDLQYSTDYTFK |
| | | AYFHNGDYPGEPFILHHSTSYNSKALIAFLAFLIIVTSIALLVVLYK |
| | | IYDL* |
| 71 | tCD45RO_F378- | ATGACCATGTATTTGTGGCTTAAACTCTTGGCATTTGGCTTTG |
| | S382 | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGAAAAATATGCAAACATCACT |
| | | GTGGATTACTTATATAACAAGGAAACTAAATTATTTACAGCA |
| | | AAGCTAAATGTTAATGAGAATGTGGAATGTGGAAACAATACT |
| | | TGCACAAACAATGAGGTGCATAACCTTACAGAATGTAAAAAT |
| | | GCGTCTGTTTCCATATCTCATAATTCATGTACTGCTCCTGATAA |
| | | GACATTAATATTAGATGTGCCACCAGGGGTTGAAAAGTTTCA |
| | | GTTACATGATTGTACACAAGTTGAAAAAGCAGATACTACTATT |
| | | TGTTTAAAATGGAAAAATATTGAAACCTTTACTTGTGATACAC |
| | | AGAATATTACCTACAGATTTCAGTGTGGTAATATGATATTTGA |
| | | TAATAAAGAAATTAAATTAGAAAACCTTGAACCCGAACATGA |
| | | GTATAAGTGTGACTCAGAAATACTCTATAATAACCACAAGGC |
| | | CGCTGCCGCAGCCAAAATTATTAAAACAGATTTTGGGAGTCC |
| | | AGGAGAGCCTCAGATTATTTTTTGTAGAAGTGAAGCTGCACAT |
| | | CAAGGAGTAATTACCTGGAATCCCCCTCAAAGATCATTTCATA |
| | | ATTTTACCCTCTGTTATATAAAAGAGACAGAAAAAGATTGCCT |
| | | CAATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 72 | tCD45RO_F378- | MTMYLWLKLLAFGFAFLDTEVFVTGEQKLISEEDLQSPTPSPTDA |
| | S382 AA | YLNASETTTLSPSGSAVISTTTIATTPSKPTCDEKYANITVDYLYN |
| | | KETKLFTAKLNVNENVECGNNTCTNNEVHNLTECKNASVSISHN |
| | | SCTAPDKTLILDVPPGVEKFQLHDCTQVEKADTTICLKWKNIETF |
| | | TCDTQNITYRFQCGNMIFDNKEIKLENLEPEHEYKCDSEILYNNH |
| | | KAAAAAKIIKTDFGSPGEPQIIFCRSEAAHQGVITWNPPQRSFHNF |
| | | TLCYIKETEKDCLNLDKNLIKYDLQNLKPYTKYVLSLHAYIIAKV |
| | | QRNGSAAMCHFTTKSAPPSQVWNMTVSMTSDNSMHVKCRPPR |
| | | DRNGPHERYHLEVEAGNTLVRNESHKNCDFRVKDLQYSTDYTF |
| | | KAYFHNGDYPGEPFILHHSTSYNSKALIAFLAFLIIVTSIALLVVLY |
| | | KIYDL* |
| 73 | tCD45RO_E230A, | ATGACCATGTATTTGTGGCTTAAACTCTTGGCATTTGGCTTTG |
| | Y232A | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGCCAAAGCTGCAAACATCACT |
| | | GTGGATTACTTATATAACAAGGAAACTAAATTATTTACAGCA |
| | | AAGCTAAATGTTAATGAGAATGTGGAATGTGGAAACAATACT |
| | | TGCACAAACAATGAGGTGCATAACCTTACAGAATGTAAAAAT |
| | | GCGTCTGTTTCCATATCTCATAATTCATGTACTGCTCCTGATAA |
| | | GACATTAATATTAGATGTGCCACCAGGGGTTGAAAAGTTTCA |
| | | GTTACATGATTGTACACAAGTTGAAAAAGCAGATACTACTATT |
| | | TGTTTAAAATGGAAAAATATTGAAACCTTTACTTGTGATACAC |
| | | AGAATATTACCTACAGATTTCAGTGTGGTAATATGATATTTGA |
| | | TAATAAAGAAATTAAATTAGAAAACCTTGAACCCGAACATGA |
| | | GTATAAGTGTGACTCAGAAATACTCTATAATAACCACAAGTTT |
| | | ACTAACGCAAGTAAAATTATTAAAACAGATTTTGGGAGTCCA |
| | | GGAGAGCCTCAGATTATTTTTTGTAGAAGTGAAGCTGCACATC |
| | | AAGGAGTAATTACCTGGAATCCCCCTCAAAGATCATTTCATAA |
| | | TTTTACCCTCTGTTATATAAAAGAGACAGAAAAAGATTGCCTC |
| | | AATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 74 | tCD45RO_E230A, | MTMYLWLKLLAFGFAFLDTEVFVTGEQKLISEEDLQSPTPSPTDA |
| | Y232A AA | YLNASETTTLSPSGSAVISTTTIATTPSKPTCDAKAANITVDYLYN |
| | | KETKLFTAKLNVNENVECGNNTCTNNEVHNLTECKNASVSISHN |
| | | SCTAPDKTLILDVPPGVEKFQLHDCTQVEKADTTICLKWKNIETF |
| | | TCDTQNITYRFQCGNMIFDNKEIKLENLEPEHEYKCDSEILYNNH |
| | | KFTNASKIIKTDFGSPGEPQIIFCRSEAAHQGVITWNPPQRSFHNFT |
| | | LCYIKETEKDCLNLDKNLIKYDLQNLKPYTKYVLSLHAYIIAKVQ |
| | | RNGSAAMCHFTTKSAPPSQVWNMTVSMTSDNSMHVKCRPPRD |
| | | RNGPHERYHLEVEAGNTLVRNESHKNCDFRVKDLQYSTDYTFK |
| | | AYFHNGDYPGEPFILHHSTSYNSKALIAFLAFLIIVTSIALLVVLYK |
| | | IYDL* |
| 75 | tCD45RO_E230A, | ATGACCATGTATTTGTGGCTTAAACTCTTGGCATTTGGCTTTG |
| | K231A | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGCCGCTTATGCAAACATCACTG |
| | | TGGATTACTTATATAACAAGGAAACTAAATTATTTACAGCAA |
| | | AGCTAAATGTTAATGAGAATGTGGAATGTGGAAACAATACTT |
| | | GCACAAACAATGAGGTGCATAACCTTACAGAATGTAAAAATG |
| | | CGTCTGTTTCCATATCTCATAATTCATGTACTGCTCCTGATAAG |
| | | ACATTAATATTAGATGTGCCACCAGGGGTTGAAAAGTTTCAGT |
| | | TACATGATTGTACACAAGTTGAAAAAGCAGATACTACTATTTG |
| | | TTTAAAATGGAAAAATATTGAAACCTTTACTTGTGATACACAG |
| | | AATATTACCTACAGATTTCAGTGTGGTAATATGATATTTGATA |
| | | ATAAAGAAATTAAATTAGAAAACCTTGAACCCGAACATGAGT |
| | | ATAAGTGTGACTCAGAAATACTCTATAATAACCACAAGTTTAC |
| | | TAACGCAAGTAAAATTATTAAAACAGATTTTGGGAGTCCAGG |
| | | AGAGCCTCAGATTATTTTTTGTAGAAGTGAAGCTGCACATCAA |
| | | GGAGTAATTACCTGGAATCCCCCTCAAAGATCATTTCATAATT |
| | | TTACCCTCTGTTATATAAAAGAGACAGAAAAAGATTGCCTCA |
| | | ATCTGGATAAAAACCTGATCAAATATGATTTGCAAAATTTAA |
| | | AACCTTATACGAAATATGTTTTATCATTACATGCCTACATCAT |
| | | TGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 76 | tCD45RO_E230A, | MTMYLWLKLLAFGFAFLDTEVFVTGEQKLISEEDLQSPTPSPTDA |
| | K231A AA | YLNASETTTLSPSGSAVISTTTIATTPSKPTCDAAYANITVDYLYN |
| | | KETKLFTAKLNVNENVECGNNTCTNNEVHNLTECKNASVSISHN |
| | | SCTAPDKTLILDVPPGVEKFQLHDCTQVEKADTTICLKWKNIETF |
| | | TCDTQNITYRFQCGNMIFDNKEIKLENLEPEHEYKCDSEILYNNH |
| | | KFTNASKIIKTDFGSPGEPQIIFCRSEAAHQGVITWNPPQRSFHNFT |
| | | LCYIKETEKDCLNLDKNLIKYDLQNLKPYTKYVLSLHAYIIAKVQ |
| | | RNGSAAMCHFTTKSAPPSQVWNMTVSMTSDNSMHVKCRPPRD |
| | | RNGPHERYHLEVEAGNTLVRNESHKNCDFRVKDLQYSTDYTFK |
| | | AYFHNGDYPGEPFILHHSTSYNSKALIAFLAFLIIVTSIALLVVLYK |
| | | IYDL* |
| 77 | tCD45RO_E230A | ATGACCATGTATTTGTGGCTTAAACTCTTGGCATTTGGCTTTG |
| | | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGCCAAATATGCAAACATCACT |
| | | GTGGATTACTTATATAACAAGGAAACTAAATTATTTACAGCA |
| | | AAGCTAAATGTTAATGAGAATGTGGAATGTGGAAACAATACT |
| | | TGCACAAACAATGAGGTGCATAACCTTACAGAATGTAAAAAT |
| | | GCGTCTGTTTCCATATCTCATAATTCATGTACTGCTCCTGATAA |
| | | GACATTAATATTAGATGTGCCACCAGGGGTTGAAAAGTTTCA |
| | | GTTACATGATTGTACACAAGTTGAAAAAGCAGATACTACTATT |
| | | TGTTTAAAATGGAAAAATATTGAAACCTTTACTTGTGATACAC |
| | | AGAATATTACCTACAGATTTCAGTGTGGTAATATGATATTTGA |
| | | TAATAAAGAAATTAAATTAGAAAACCTTGAACCCGAACATGA |
| | | GTATAAGTGTGACTCAGAAATACTCTATAATAACCACAAGTTT |
| | | ACTAACGCAAGTAAAATTATTAAAACAGATTTTGGGAGTCCA |
| | | GGAGAGCCTCAGATTATTTTTTGTAGAAGTGAAGCTGCACATC |
| | | AAGGAGTAATTACCTGGAATCCCCCTCAAAGATCATTTCATAA |
| | | TTTTACCCTCTGTTATATAAAAGAGACAGAAAAAGATTGCCTC |
| | | AATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 78 | tCD45RO_E230A | MTMYLWLKLLAFGFAFLDTEVFVTGEQKLISEEDLQSPTPSPTDA |
| | AA | YLNASETTTLSPSGSAVISTTTIATTPSKPTCDAKYANITVDYLYN |
| | | KETKLFTAKLNVNENVECGNNTCTNNEVHNLTECKNASVSISHN |
| | | SCTAPDKTLILDVPPGVEKFQLHDCTQVEKADTTICLKWKNIETF |
| | | TCDTQNITYRFQCGNMIFDNKEIKLENLEPEHEYKCDSEILYNNH |
| | | KFTNASKIIKTDFGSPGEPQIIFCRSEAAHQGVITWNPPQRSFHNFT |
| | | LCYIKETEKDCLNLDKNLIKYDLQNLKPYTKYVLSLHAYIIAKVQ |
| | | RNGSAAMCHFTTKSAPPSQVWNMTVSMTSDNSMHVKCRPPRD |
| | | RNGPHERYHLEVEAGNTLVRNESHKNCDFRVKDLQYSTDYTFK |
| | | AYFHNGDYPGEPFILHHSTSYNSKALIAFLAFLIIVTSIALLVVLYK |
| | | IYDL* |
| 79 | tCD45RO_D334- | ATGACCATGTATTTGTGGCTTAAACTCTTGGCATTTGGCTTTG |
| | N337 | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGATGAAAAATATGCAAACATCACT |
| | | GTGGATTACTTATATAACAAGGAAACTAAATTATTTACAGCA |
| | | AAGCTAAATGTTAATGAGAATGTGGAATGTGGAAACAATACT |
| | | TGCACAAACAATGAGGTGCATAACCTTACAGAATGTAAAAAT |
| | | GCGTCTGTTTCCATATCTCATAATTCATGTACTGCTCCTGATAA |
| | | GACATTAATATTAGATGTGCCACCAGGGGTTGAAAAGTTTCA |
| | | GTTACATGATTGTACACAAGTTGAAAAAGCAGATACTACTATT |
| | | TGTTTAAAATGGAAAAATATTGAAACCTTTACTTGTGCCGCAG |
| | | CGGCTATTACCTACAGATTTCAGTGTGGTAATATGATATTTGA |
| | | TAATAAAGAAATTAAATTAGAAAACCTTGAACCCGAACATGA |
| | | GTATAAGTGTGACTCAGAAATACTCTATAATAACCACAAGTTT |
| | | ACTAACGCAAGTAAAATTATTAAAACAGATTTTGGGAGTCCA |
| | | GGAGAGCCTCAGATTATTTTTTGTAGAAGTGAAGCTGCACATC |
| | | AAGGAGTAATTACCTGGAATCCCCCTCAAAGATCATTTCATAA |
| | | TTTTACCCTCTGTTATATAAAAGAGACAGAAAAAGATTGCCTC |
| | | AATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 80 | tCD45RO_D334- | MTMYLWLKLLAFGFAFLDTEVFVTGEQKLISEEDLQSPTPSPTDA |
| | N337 | YLNASETTTLSPSGSAVISTTTIATTPSKPTCDEKYANITVDYLYN |
| | | KETKLFTAKLNVNENVECGNNTCTNNEVHNLTECKNASVSISHN |
| | | SCTAPDKTLILDVPPGVEKFQLHDCTQVEKADTTICLKWKNIETF |
| | | TCAAAAITYRFQCGNMIFDNKEIKLENLEPEHEYKCDSEILYNNH |
| | | KFTNASKIIKTDFGSPGEPQIIFCRSEAAHQGVITWNPPQRSFHNFT |
| | | LCYIKETEKDCLNLDKNLIKYDLQNLKPYTKYVLSLHAYIIAKVQ |
| | | RNGSAAMCHFTTKSAPPSQVWNMTVSMTSDNSMHVKCRPPRD |
| | | RNGPHERYHLEVEAGNTLVRNESHKNCDFRVKDLQYSTDYTFK |
| | | AYFHNGDYPGEPFILHHSTSYNSKALIAFLAFLIIVTSIALLVVLYK |
| | | IYDL* |
| 81 | tCD45RO_D229A, | ATGACCATGTATTTGTGGCTTAAACTCTTGGCATTTGGCTTTG |
| | Y232A | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGCCGAAAAAGCCGCAAACATCACT |
| | | GTGGATTACTTATATAACAAGGAAACTAAATTATTTACAGCA |
| | | AAGCTAAATGTTAATGAGAATGTGGAATGTGGAAACAATACT |
| | | TGCACAAACAATGAGGTGCATAACCTTACAGAATGTAAAAAT |
| | | GCGTCTGTTTCCATATCTCATAATTCATGTACTGCTCCTGATAA |
| | | GACATTAATATTAGATGTGCCACCAGGGGTTGAAAAGTTTCA |
| | | GTTACATGATTGTACACAAGTTGAAAAAGCAGATACTACTATT |
| | | TGTTTAAAATGGAAAAATATTGAAACCTTTACTTGTGATACAC |
| | | AGAATATTACCTACAGATTTCAGTGTGGTAATATGATATTTGA |
| | | TAATAAAGAAATTAAATTAGAAAACCTTGAACCCGAACATGA |
| | | GTATAAGTGTGACTCAGAAATACTCTATAATAACCACAAGTTT |
| | | ACTAACGCAAGTAAAATTATTAAAACAGATTTTGGGAGTCCA |
| | | GGAGAGCCTCAGATTATTTTTTGTAGAAGTGAAGCTGCACATC |
| | | AAGGAGTAATTACCTGGAATCCCCCTCAAAGATCATTTCATAA |
| | | TTTTACCCTCTGTTATATAAAAGAGACAGAAAAAGATTGCCTC |
| | | AATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 82 | tCD45RO_D229A, | MTMYLWLKLLAFGFAFLDTEVFVTGEQKLISEEDLQSPTPSPTDA |
| | Y232A AA | YLNASETTTLSPSGSAVISTTTIATTPSKPTCAEKAANITVDYLYN |
| | | KETKLFTAKLNVNENVECGNNTCTNNEVHNLTECKNASVSISHN |
| | | SCTAPDKTLILDVPPGVEKFQLHDCTQVEKADTTICLKWKNIETF |
| | | TCDTQNITYRFQCGNMIFDNKEIKLENLEPEHEYKCDSEILYNNH |
| | | KFTNASKIIKTDFGSPGEPQIIFCRSEAAHQGVITWNPPQRSFHNFT |
| | | LCYIKETEKDCLNLDKNLIKYDLQNLKPYTKYVLSLHAYIIAKVQ |
| | | RNGSAAMCHFTTKSAPPSQVWNMTVSMTSDNSMHVKCRPPRD |
| | | RNGPHERYHLEVEAGNTLVRNESHKNCDFRVKDLQYSTDYTFK |
| | | AYFHNGDYPGEPFILHHSTSYNSKALIAFLAFLIIVTSIALLVVLYK |
| | | IYDL* |
| 83 | tCD45RO_D229A, | ATGACCATGTATTTGTGGCTTAAACTCTTGGCATTTGGCTTTG |
| | E230A | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGCCGCAAAATATGCAAACATCACT |
| | | GTGGATTACTTATATAACAAGGAAACTAAATTATTTACAGCA |
| | | AAGCTAAATGTTAATGAGAATGTGGAATGTGGAAACAATACT |
| | | TGCACAAACAATGAGGTGCATAACCTTACAGAATGTAAAAAT |
| | | GCGTCTGTTTCCATATCTCATAATTCATGTACTGCTCCTGATAA |
| | | GACATTAATATTAGATGTGCCACCAGGGGTTGAAAAGTTTCA |
| | | GTTACATGATTGTACACAAGTTGAAAAAGCAGATACTACTATT |
| | | TGTTTAAAATGGAAAAATATTGAAACCTTTACTTGTGATACAC |
| | | AGAATATTACCTACAGATTTCAGTGTGGTAATATGATATTTGA |
| | | TAATAAAGAAATTAAATTAGAAAACCTTGAACCCGAACATGA |
| | | GTATAAGTGTGACTCAGAAATACTCTATAATAACCACAAGTTT |
| | | ACTAACGCAAGTAAAATTATTAAAACAGATTTTGGGAGTCCA |
| | | GGAGAGCCTCAGATTATTTTTTGTAGAAGTGAAGCTGCACATC |
| | | AAGGAGTAATTACCTGGAATCCCCCTCAAAGATCATTTCATAA |
| | | TTTTACCCTCTGTTATATAAAAGAGACAGAAAAAGATTGCCTC |
| | | AATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 84 | tCD45RO_D229A, | MTMYLWLKLLAFGFAFLDTEVFVTGEQKLISEEDLQSPTPSPTDA |
| | E230A AA | YLNASETTTLSPSGSAVISTTTIATTPSKPTCAAKYANITVDYLYN |
| | | KETKLFTAKLNVNENVECGNNTCTNNEVHNLTECKNASVSISHN |
| | | SCTAPDKTLILDVPPGVEKFQLHDCTQVEKADTTICLKWKNIETF |
| | | TCDTQNITYRFQCGNMIFDNKEIKLENLEPEHEYKCDSEILYNNH |
| | | KFTNASKIIKTDFGSPGEPQIIFCRSEAAHQGVITWNPPQRSFHNFT |
| | | LCYIKETEKDCLNLDKNLIKYDLQNLKPYTKYVLSLHAYIIAKVQ |
| | | RNGSAAMCHFTTKSAPPSQVWNMTVSMTSDNSMHVKCRPPRD |
| | | RNGPHERYHLEVEAGNTLVRNESHKNCDFRVKDLQYSTDYTFK |
| | | AYFHNGDYPGEPFILHHSTSYNSKALIAFLAFLIIVTSIALLVVLYK |
| | | IYDL* |
| 85 | tCD45RO_D229A, | ATGACCATGTATTTGTGGCTTAAACTCTTGGCATTTGGCTTTG |
| | K232A | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGCCGAAGCCTATGCAAACATCACT |
| | | GTGGATTACTTATATAACAAGGAAACTAAATTATTTACAGCA |
| | | AAGCTAAATGTTAATGAGAATGTGGAATGTGGAAACAATACT |
| | | TGCACAAACAATGAGGTGCATAACCTTACAGAATGTAAAAAT |
| | | GCGTCTGTTTCCATATCTCATAATTCATGTACTGCTCCTGATAA |
| | | GACATTAATATTAGATGTGCCACCAGGGGTTGAAAAGTTTCA |
| | | GTTACATGATTGTACACAAGTTGAAAAAGCAGATACTACTATT |
| | | TGTTTAAAATGGAAAAATATTGAAACCTTTACTTGTGATACAC |
| | | AGAATATTACCTACAGATTTCAGTGTGGTAATATGATATTTGA |
| | | TAATAAAGAAATTAAATTAGAAAACCTTGAACCCGAACATGA |
| | | GTATAAGTGTGACTCAGAAATACTCTATAATAACCACAAGTTT |
| | | ACTAACGCAAGTAAAATTATTAAAACAGATTTTGGGAGTCCA |
| | | GGAGAGCCTCAGATTATTTTTTGTAGAAGTGAAGCTGCACATC |
| | | AAGGAGTAATTACCTGGAATCCCCCTCAAAGATCATTTCATAA |
| | | TTTTACCCTCTGTTATATAAAAGAGACAGAAAAAGATTGCCTC |
| | | AATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 86 | tCD45RO_D229A, | MTMYLWLKLLAFGFAFLDTEVFVTGEQKLISEEDLQSPTPSPTDA |
| | K232A AA | YLNASETTTLSPSGSAVISTTTIATTPSKPTCAEAYANITVDYLYN |
| | | KETKLFTAKLNVNENVECGNNTCTNNEVHNLTECKNASVSISHN |
| | | SCTAPDKTLILDVPPGVEKFQLHDCTQVEKADTTICLKWKNIETF |
| | | TCDTQNITYRFQCGNMIFDNKEIKLENLEPEHEYKCDSEILYNNH |
| | | KFTNASKIIKTDFGSPGEPQIIFCRSEAAHQGVITWNPPQRSFHNFT |
| | | LCYIKETEKDCLNLDKNLIKYDLQNLKPYTKYVLSLHAYIIAKVQ |
| | | RNGSAAMCHFTTKSAPPSQVWNMTVSMTSDNSMHVKCRPPRD |
| | | RNGPHERYHLEVEAGNTLVRNESHKNCDFRVKDLQYSTDYTFK |
| | | AYFHNGDYPGEPFILHHSTSYNSKALIAFLAFLIIVTSIALLVVLYK |
| | | IYDL* |
| 87 | tCD45RO_D229A | ATGACCATGTATTTGTGGCTTAAACTCTTGGCATTTGGCTTTG |
| | | CCTTTCTGGACACAGAAGTATTTGTGACAGGGGAACAAAAAC |
| | | TCATCTCAGAAGAGGATCTGCAAAGCCCAACACCTTCCCCCA |
| | | CTGATGCCTACCTTAATGCCTCTGAAACAACCACTCTGAGCCC |
| | | TTCTGGAAGCGCTGTCATTTCAACCACAACAATAGCTACTACT |
| | | CCATCTAAGCCAACATGTGCCGAAAAATATGCAAACATCACT |
| | | GTGGATTACTTATATAACAAGGAAACTAAATTATTTACAGCA |
| | | AAGCTAAATGTTAATGAGAATGTGGAATGTGGAAACAATACT |
| | | TGCACAAACAATGAGGTGCATAACCTTACAGAATGTAAAAAT |
| | | GCGTCTGTTTCCATATCTCATAATTCATGTACTGCTCCTGATAA |
| | | GACATTAATATTAGATGTGCCACCAGGGGTTGAAAAGTTTCA |
| | | GTTACATGATTGTACACAAGTTGAAAAAGCAGATACTACTATT |
| | | TGTTTAAAATGGAAAAATATTGAAACCTTTACTTGTGATACAC |
| | | AGAATATTACCTACAGATTTCAGTGTGGTAATATGATATTTGA |
| | | TAATAAAGAAATTAAATTAGAAAACCTTGAACCCGAACATGA |
| | | GTATAAGTGTGACTCAGAAATACTCTATAATAACCACAAGTTT |
| | | ACTAACGCAAGTAAAATTATTAAAACAGATTTTGGGAGTCCA |
| | | GGAGAGCCTCAGATTATTTTTTGTAGAAGTGAAGCTGCACATC |
| | | AAGGAGTAATTACCTGGAATCCCCCTCAAAGATCATTTCATAA |
| | | TTTTACCCTCTGTTATATAAAAGAGACAGAAAAAGATTGCCTC |
| | | AATCTGGATAAAAACCTGATCAAATATGATTTGCAAAATTTA |
| | | AAACCTTATACGAAATATGTTTTATCATTACATGCCTACATCA |
| | | TTGCAAAAGTGCAACGTAATGGAAGTGCTGCAATGTGTCATTT |
| | | CACAACTAAAAGTGCTCCTCCAAGCCAGGTCTGGAACATGAC |
| | | TGTCTCCATGACATCAGATAATAGTATGCATGTCAAGTGTAGG |
| | | CCTCCCAGGGACCGTAATGGCCCCCATGAACGTTACCATTTGG |
| | | AAGTTGAAGCTGGAAATACTCTGGTTAGAAATGAGTCGCATA |
| | | AGAATTGCGATTTCCGTGTAAAAGATCTTCAATATTCAACAGA |
| | | CTACACTTTTAAGGCCTATTTTCACAATGGAGACTATCCTGGA |
| | | GAACCCTTTATTTTACATCATTCAACATCTTATAATTCTAAGG |
| | | CACTGATAGCATTTCTGGCATTTCTGATTATTGTGACATCAAT |
| | | AGCCCTGCTTGTTGTTCTCTACAAAATCTATGATCTATAA |
| 88 | tCD45RO_D229A | MTMYLWLKLLAFGFAFLDTEVFVTGEQKLISEEDLQSPTPSPTDA |
| | AA | YLNASETTTLSPSGSAVISTTTIATTPSKPTCAEKYANITVDYLYN |
| | | KETKLFTAKLNVNENVECGNNTCTNNEVHNLTECKNASVSISHN |
| | | SCTAPDKTLILDVPPGVEKFQLHDCTQVEKADTTICLKWKNIETF |
| | | TCDTQNITYRFQCGNMIFDNKEIKLENLEPEHEYKCDSEILYNNH |
| | | KFTNASKIIKTDFGSPGEPQIIFCRSEAAHQGVITWNPPQRSFHNFT |
| | | LCYIKETEKDCLNLDKNLIKYDLQNLKPYTKYVLSLHAYIIAKVQ |
| | | RNGSAAMCHFTTKSAPPSQVWNMTVSMTSDNSMHVKCRPPRD |
| | | RNGPHERYHLEVEAGNTLVRNESHKNCDFRVKDLQYSTDYTFK |
| | | AYFHNGDYPGEPFILHHSTSYNSKALIAFLAFLIIVTSIALLVVLYK |
| | | IYDL* |

TABLE 2
Anti-CD45 CAR Sequences

| SEQ | | |
|---|---|---|
| ID | | |
| NO: | Description | Sequence |
| 89 | 4B2 HC CDR1 | SYTMQ |
| 90 | 4B2 HC CDR2 | YINPSSGYIKYNQKFKD |
| 91 | 4B2 HC CDR3 | RGSYFFDF |
| 92 | 9.4 HC CDR1 | SYSIQ |
| 93 | 9.4 HC CDR2 | YINPSSGYIKYNQHFRD |
| 94 | 9.4 HC CDR3 | GNSGSFDY |
| 95 | 4122 HC CDR1 | GFSFSAGYWIC |
| 96 | 4122 HC CDR2 | CTYAGRSGSTYYANWVNG |
| 97 | 4122 HC CDR3 | GNAGVAVGAL |
| 98 | BC8 HC CDR1 | GFDFSRYWMS |
| 99 | BC8 HC CDR2 | EINPTSSTINFTPSLKD |
| 100 | BC8 HC CDR3 | GNYYRYGDAM |
| 101 | CN1 HC CDR1 | GYTFSGYA |
| 102 | CN1 HC CDR2 | INPKSPGT |
| 103 | CN1 HC CDR3 | ARYSSSSWGDP |
| 104 | Gap8.3 HC CDR1 | GYFIH |
| 105 | Gap8.3 HC CDR2 | YISCYNGATSYNQKFKG |
| 106 | Gap8.3 HC CDR3 | NYYGNLDAMDY |
| 107 | mAb104 HC CDR1 | FSNYGMS |
| 108 | mAb104 HC CDR2 | TIVGNDYTYFPDSM |
| 109 | mAb104 HC CDR3 | HDWVFDY |
| 110 | NOV45 HC CDR1 | NYIIH |
| 111 | NOV45 HC CDR2 | FNPYNHGTKYNEKFKG |
| 112 | NOV45 HC CDR3 | ARSGPYAWF |
| 113 | muUCHL HC CDR1 | GFSLTTY |
| 114 | muUCHL HC CDR2 | IWWNDNK |
| 115 | muUCHL HC CDR3 | LYGYTY |
| 116 | huUCHL HC CDR1 | GFSLTTY |
| 117 | huUCHL HC CDR2 | IWWNDNK |
| 118 | huUCHL HC CDR3 | LYGYTY |
| 119 | 4B2 LC CDR1 | RSSKSLLYKDGKTYLN |
| 120 | 4B2 LC CDR2 | LMSTRAS |
| 121 | 4B2 LC CDR3 | QQLVEYPFT |
| 122 | 9.4 LC CDR1 | RSSKSLLHSSGITYLY |
| 123 | 9.4 LC CDR2 | RMSNLAS |
| 124 | 9.4 LC CDR3 | MQHLEYPFT |
| 125 | 4122 LC CDR1 | QASQSISNWLA |
| 126 | 4122 LC CDR2 | QASKLAS |
| 127 | 4122 LC CDR3 | QSYYDSGSNVFFA |
| 128 | BC8 LC CDR1 | RASKSVSTSGYSYLH |
| 129 | BC8 LC CDR2 | LASNLES |
| 130 | BC8 LC CDR3 | QHSRELPFT |
| 131 | CN1 LC CDR1 | AQPKAY |
| 132 | CN1 LC CDR2 | GDS |
| 133 | CN1 LC CDR3 | YSTDRSGNRV |
| 134 | Gap8.3 LC CDR1 | KASQDVSTAVA |
| 135 | Gap8.3 LC CDR2 | SASYRYT |
| 136 | Gap8.3 LC CDR3 | QQHYSTPRT |
| 137 | mAb104 LC CDR1 | KASQSVSFAGSSLMH |
| 138 | mAb104 LC CDR2 | RASDLET |
| 139 | mAb104 LC CDR3 | QQSREYPYT |
| 140 | NOV45 LC CDR1 | RASQNIGTSIQ |
| 141 | NOV45 LC CDR2 | SSSESIS |
| 142 | NOV45 LC CDR3 | QQSNTWPFT |
| 143 | Hu/muUCHL LC | QSLLYSNGNTY |
| | CDR1 | |
| 144 | Hu/muUCHL LC | KLS |
| | CDR2 | |
| 145 | Hu/muUCHL LC | SQSTHVPWT |
| | CDR3 | |
| 146 | 4B2 HC | GVQLQESGAELARPGASVKMSCKASGYTFTSYTMQW |
| | | VKQRPGQGLEWIGYINPSSGYIKYNQKFKDKVTLTAD |
| | | KSSTTAYMQLSRLTSEDSAVYYCARRGSYFFDFWGQG |
| | | TSVTVSS |
| 147 | 9.4 HC | QVQLQQLGAELARPGASVKMSCKASGYTFTSYSIQWV |
| | | KQRPGQGLEWIGYINPSSGYIKYNQHFRDRATLTADRS |
| | | SSTAYMQLSSLTSEDSAVYYCARGNSGSFDYWGQGTT |
| | | LTVSS |
| 148 | 4122 HC | LSLEESGGDLVKPGASLTLTCTASGFSFSAGYWICWV |
| | | RQAPGKGLEWIACTYAGRSGSTYYANWVNGRFTIPK |
| | | TSSTTVTLQMTSLSGADTASYFCARGNAGVAVGALW |
| | | GPGTLVTVSS |
| 149 | BC8 HC | QVQLVESGGGLVQPGGSLKLSCAASGFDFSRYWMSW |
| | | VRQAPGKGLEWIGEINPTSSTINFTPSLKDKVFISRDNA |
| | | KNTLYLQMSKVRSEDTALYYCARGNYYRYGDAMDY |
| | | WGQGTSVTVS |
| 150 | CN1 HC | QVQLVQSGAEVKKPGASVKVSCKASGYTFSGYAMHW |
| | | VRQAPGQGLEWMGWINPKSPGTNYAQKFQGRVTMTR |
| | | DTSISTAYMELSRLRSDDTAVYYCARYSSSSWGDPWG |
| | | QGTLVTVSS |
| 151 | Gap8.3 HC | EVQLQQSGPELVKTGASVKISCKASGYSFTGYFIHWVK |
| | | QSHGKSLEWIGYISCYNGATSYNQKFKGKATFTVDTSS |
| | | STAYMQFNSVTSEDSAVYYCVRNYYGNLDAMDYWG |
| | | QGTSVTVSS |
| 152 | mAB104 HC | EVQLVESGGDLVQPGGSLKLSCTASGFTFSNYGMSWIR |
| | | QTPDKRLEWVATIVGNDYTYFPDSMKGRFTVSRDNAK |
| | | SILYLQMNSLASADTAMYYCTRHDWVFDYWGQGTPL |
| | | TVSS |
| 153 | NOV45 HC | QVQLVESGAEVKKPGASVKVSCKASGYTFTNYIIHWV |
| | | KQEPGQGLEWIGYFNPYNHGTKYNEKFKGRATLTADK |
| | | SISTAYMELSSLRSEDTAVYYCARSGPYAWFDTWGQG |
| | | TTVTVSS |
| 154 | muUCHL HC | QITLKESGPGILQPSQTLSLTCSFSGFSLTTYGIGVGWIR |
| | | QPPGKGLEWLTHIWWNDNKYYNTALRSRLTISKDSSN |
| | | NQVLLKIANVDTADTATYYCLYGYTYWGQGTLVTVS |
| | | A |
| 155 | huUCHL HC | QITLKESGPTLVKPTQTLTLTCTFSGFSLTTYGIGVGWI |
| | | RQPPGKALEWLTHIWWNDNKYYSPSLKSRLTITKDSS |
| | | KNQVVLTMTNMDPVDTATYYCLYGYTYWGQGTLVT |
| | | VSA |
| 156 | 4B2 LC | DIVITQDELSNPVTSGESVSISCRSSKSLLYKDGKTYLN |
| | | WFLQRPGQSPQLLIYLMSTRASGVSDRFSGSGSGTDFT |
| | | LEISRVKAEDVGVYYCQQLVEYPFTFGGGTKLEVK |
| 157 | 9.4 LC | DIVMTQAAPSVPVTPGESLSISCRSSKSLLHSSGITYLY |
| | | WFLQRPGQSPQLLIYRMSNLASGVPDRFSGSGSGTAFT |
| | | LRISRVEAEDVGVYYCMQHLEYPFTFGGGTKLEIK |
| 158 | 4122 LC | DIVMTQTPASVSEPVGGTVTIMCQASQSISNWLAWYQ |
| | | QKPGQPPKLLIYQASKLASGVPSRFKGSGSGTEYTLTIS |
| | | DLECADAATYYCQSYYDSGSNVFFAFGGGTKVVVE |
| 159 | BC8 LC | DIVLTQSPASLAVSLGQRATISCRASKSVSTSGYSYLH |
| | | WYQQKPGQPPKLLIYLASNLESGVPARFSGSGSGTDFT |
| | | LNIHPVEEEDAATYYCQHSRELPFTFGSGTKLEIK |
| 160 | CN1 LC | SYELTQPPSVSVSPGQTARVTCSGDAQPKAYAYWYQQ |
| | | KSGQAPILVITGDSKRPSGIPERFSGSSSGTMATLTISGA |
| | | QVEDEADYYCYSTDRSGNRVFGGGTRLTV |
| 161 | Gap8.3 LC | DIVMTQSHKFMSTSVGDRVSITCKASQDVSTAVAWYQ |
| | | QKPGQSPKILIYSASYRYTGVPDRFTGSGSGTDFTFTISS |
| | | VQAEDLAVYYCQQHYSTPRTFGGGTKLEIK |
| 162 | mAB104 LC | DIVLTQSPASLAVSLGQRAILSCKASQSVSFAGSSLMH |
| | | WYQQKPGQQPKLLIYRASDLETGIPTRFSGGGSGTDFT |
| | | LNIHPVEEDDAATYYCQQSREYPYTFGGGTRLEIK |
| 163 | NOV45 LC | DILLTQSPATLSLSPGERATLSCRASQNIGTSIQWYQQK |
| | | PGQAPRLLIRSSSESISGISSRFSGSGSGTDFTLTISSLEPE |
| | | DFAVYYCQQSNTWPFTFGQGTKLEIK |
| 164 | muUCHL LC | DVVMTQTPLSLPVSLGDQASISCRSSQSLLYSNGNTYL |
| | | HWYLQKPGQSPKLLIYKLSNRFSGVPDRFSGSGSGTDF |
| | | TLKISRVEAEDLGVYFCSQSTHVPWTFGGGTKLEIK |
| 165 | huUCHL LC | DVVMTQTPLSLPVTLGQPASISCRSSQSLLYSNGNTYL |
| | | HWYQQRPGQSPRLLIYKLSNRFSGVPDRFSGSGSGTDF |
| | | TLKISRVEAEDVGVYYCSQSTHVPWTFGGGTKLEIK |
| 166 | CD8 Leader | MALPVTALLLPLALLLHAARP |
| | Sequence | |
| 167 | (G4S)₄ Linker | GGGGSGGGGSGGGGSGGGGS |
| 168 | CD8 Hinge | TTTPAPRPPTPAPTIASQPLSLRPEACRPAAGGAVHTRG |
| | | LDFACD |
| 169 | CD8 | IYIWAPLAGTCGVLLLSLVITLYC |
| | Transmembrane | |
| | Domain | |
| 170 | 41BB Costimulatory | KRGRKKLLYIFKQPFMRPVQTTQEEDGCSCRFPEEEEG |
| | Domain | GCEL |
| 171 | CD3zeta Signaling | RVKFSRSADAPAYKQGQNQLYNELNLGRREEYDVLD |
| | Domain | KRRGRDPEMGGKPRRKNPQEGLYNELQKDKMAEAYS |
| | | EIGMKGERRRGKGHDGLYQGLSTATKDTYDALHMQA |
| | | LPPR |
| 172 | P2A Ribosomal Skip | GSGATNFSLLKQAGDVEENPGP |
| | Element | |
| 173 | Truncated NGFR | MGAGATGRAMDGPRLLLLLLLGVSLGGAKEACPTGL |
| | | YTHSGECCKACNLGEGVAQPCGANQTVCEPCLDSVTF |
| | | SDVVSATEPCKPCTECVGLQSMSAPCVEADDAVCRCA |
| | | YGYYQDETTGRCEACRVCEAGSGLVFSCQDKQNTVCE |
| | | ECPDGTYSDEANHVDPCLPCTVCEDTERQLRECTRWA |
| | | DAECEEIPGRWITRSTPPEGSDSTAPSTQEPEAPPEQDLI |
| | | ASTVAGVVTTVMGSSQPVVTRGTTDNLIPVYCSILAAV |
| | | VVGLVAYIAFKRWNS |
| 174 | 4B2 VH-VL | ATGGCGCTCCCTGTGACAGCCCTGCTGCTGCCTTTGG |
| | | CGCTGCTGCTTCACGCCGCTCGTCCGGGGGTTCAGTT |
| | | ACAGGAGTCGGGAGCGGAGCTGGCCCGCCCTGGCG |
| | | CCTCCGTGAAGATGTCTTGCAAGGCCAGCGGCTACA |
| | | CCTTCACATCCTACACTATGCAGTGGGTCAAACAGC |
| | | GGCCCGGCCAGGGACTGGAGTGGATCGGTTATATTA |
| | | ATCCCTCTTCTGGCTACATCAAGTACAACCAGAAAT |
| | | TTAAGGACAAGGTCACCCTGACAGCGGATAAAAGCT |
| | | CCACCACCGCTTACATGCAGCTGTCCCGTCTGACCTC |
| | | CGAGGATTCCGCCGTGTACTATTGCGCCCGTCGCGG |
| | | GAGCTACTTCTTCGATTTTTGGGGCCAGGGTACAAG |
| | | CGTCACAGTGTCTTCCGGCGGCGGTGGCTCGGGTGG |
| | | AGGCGGCTCCGGCGGTGGTGGTAGCGGAGGCGGCG |
| | | GTTCAGACATCGTGATCACCCAGGACGAGTTGTCAA |
| | | ATCCAGTGACTAGTGGAGAGTCCGTGTCCATCTCCT |
| | | GTCGCTCCTCCAAGTCGCTGTTATATAAGGATGGCA |
| | | AGACTTACCTGAACTGGTTCCTGCAGCGTCCTGGGC |
| | | AGAGCCCCCAGCTCCTTATCTACCTGATGTCTACGCG |
| | | AGCCAGTGGCGTGTCAGACCGCTTTTCCGGTTCCGG |
| | | CAGCGGGACCGACTTCACTCTCGAGATCTCCAGGGT |
| | | GAAGGCTGAGGACGTAGGCGTGTACTATTGTCAGCA |
| | | GCTCGTGGAGTACCCTTTCACCTTCGGCGGCGGCAC |
| | | CAAACTGGAGGTGAAGACCACTACCCCCGCTCCCCG |
| | | GCCGCCGACCCCCGCCCCTACCATCGCCTCTCAGCC |
| | | ACTTTCGCTACGCCCTGAGGCTTGTCGGCCCGCTGCC |
| | | GGGGGCGCGGTGCACACCCGCGGCCTGGACTTCGCC |
| | | TGCGACATCTACATCTGGGCGCCGCTGGCCGGCACC |
| | | TGCGGGGTCCTGTTGCTTTCTCTGGTGATCACGCTGT |
| | | ATTGTAAGAGAGGCCGCAAGAAGCTGCTGTATATCT |
| | | TCAAACAGCCCTTCATGCGCCCAGTGCAGACGACCC |
| | | AGGAGGAAGATGGATGCTCTTGCCGCTTCCCCGAAG |
| | | AGGAGGAGGGCGGGTGCGAGCTCAGAGTGAAATTT |
| | | TCCCGAAGCGCGGACGCTCCGGCATACAAACAAGG |
| | | ACAGAATCAGCTGTACAACGAGTTGAACTTGGGTCG |
| | | CCGGGAGGAATACGACGTGCTGGACAAGCGCCGCG |
| | | GCAGGGACCCTGAGATGGGAGGGAAGCCCCGCCGC |
| | | AAGAACCCCCAGGAGGGCCTGTACAACGAGCTGCA |
| | | GAAGGACAAAATGGCCGAAGCCTACTCCGAGATTG |
| | | GTATGAAGGGCGAGCGGCGCAGAGGCAAGGGTCAC |
| | | GACGGCCTGTACCAGGGCTTGTCTACCGCCACTAAG |
| | | GACACCTACGATGCGCTGCACATGCAGGCACTGCCT |
| | | CCCCGCGGATCGGGAGCTACCAACTTTTCACTGCTC |
| | | AAGCAGGCGGGCGACGTGGAGGAGAACCCGGGACC |
| | | GATGGGCGCGGGCGCCACGGGACGTGCCATGGATG |
| | | GTCCCCGTCTACTTCTTCTGTTGCTACTCGGCGTGTC |
| | | TCTTGGGGGCGCCAAGGAGGCCTGCCCGACCGGGTT |
| | | GTACACCCATAGCGGCGAGTGTTGCAAGGCCTGTAA |
| | | CCTGGGCGAGGGCGTCGCTCAGCCCTGTGGAGCTAA |
| | | CCAGACCGTCTGTGAGCCTTGCCTGGATAGCGTGAC |
| | | CTTCAGTGATGTGGTTTCCGCCACCGAGCCATGCAA |
| | | GCCCTGCACTGAGTGCGTGGGACTCCAGTCGATGTC |
| | | TGCACCTTGCGTGGAGGCCGATGACGCCGTGTGCCG |
| | | CTGTGCTTACGGCTACTACCAAGACGAGACCACCGG |
| | | TAGGTGCGAGGCATGCCGCGTCTGTGAAGCGGGAAG |
| | | CGGACTGGTGTTCAGCTGTCAGGACAAGCAGAACAC |
| | | CGTTTGTGAGGAGTGCCCAGACGGCACCTATAGTGA |
| | | CGAGGCCAATCATGTGGACCCCTGCCTGCCCTGTAC |
| | | CGTGTGCGAGGACACCGAGCGCCAGCTCCGCGAGTG |
| | | TACTCGCTGGGCGGACGCAGAATGCGAGGAGATTCC |
| | | GGGTCGCTGGATAACGAGGTCCACTCCGCCTGAAGG |
| | | CTCCGACAGTACGGCTCCGAGTACCCAGGAGCCCGA |
| | | GGCTCCACCCGAACAGGACCTGATCGCCTCTACCGT |
| | | GGCTGGCGTCGTTACTACCGTGATGGGCTCCTCCCA |
| | | ACCAGTAGTCACCCGCGGCACTACTGACAACCTTAT |
| | | TCCGGTGTACTGTTCCATCCTGGCAGCAGTCGTTGTG |
| | | GGGCTGGTGGCGTACATTGCGTTCAAGCGATGGAAC |
| | | TCCTGA |
| 175 | 4B2 VH-VL AA | MALPVTALLLPLALLLHAARPGVQLQESGAELARPGA |
| | | SVKMSCKASGYTFTSYTMQWVKQRPGQGLEWIGYINP |
| | | SSGYIKYNQKFKDKVTLTADKSSTTAYMQLSRLTSEDS |
| | | AVYYCARRGSYFFDFWGQGTSVTVSSGGGGSGGGGS |
| | | GGGGSGGGGSDIVITQDELSNPVTSGESVSISCRSSKSL |
| | | LYKDGKTYLNWFLQRPGQSPQLLIYLMSTRASGVSDR |
| | | FSGSGSGTDFTLEISRVKAEDVGVYYCQQLVEYPFTFG |
| | | GGTKLEVKTTTPAPRPPTPAPTIASQPLSLRPEACRPAA |
| | | GGAVHTRGLDFACDIYIWAPLAGTCGVLLLSLVITLYC |
| | | KRGRKKLLYIFKQPFMRPVQTTQEEDGCSCRFPEEEEG |
| | | GCELRVKFSRSADAPAYKQGQNQLYNELNLGRREEYD |
| | | VLDKRRGRDPEMGGKPRRKNPQEGLYNELQKDKMAE |
| | | AYSEIGMKGERRRGKGHDGLYQGLSTATKDTYDALH |
| | | MQALPPRGSGATNFSLLKQAGDVEENPGPMGAGATG |
| | | RAMDGPRLLLLLLLGVSLGGAKEACPTGLYTHSGECC |
| | | KACNLGEGVAQPCGANQTVCEPCLDSVTFSDVVSATE |
| | | PCKPCTECVGLQSMSAPCVEADDAVCRCAYGYYQDE |
| | | TTGRCEACRVCEAGSGLVFSCQDKQNTVCEECPDGTY |
| | | SDEANHVDPCLPCTVCEDTERQLRECTRWADAECEEIP |
| | | GRWITRSTPPEGSDSTAPSTQEPEAPPEQDLIASTVAGV |
| | | VTTVMGSSQPVVTRGTTDNLIPVYCSILAAVVVGLVA |
| | | YIAFKRWNS* |
| 176 | 4B2 VL-VH | ATGGCGCTGCCCGTGACTGCTCTGCTGTTACCGCTGG |
| | | CGTTGTTGCTACACGCCGCCAGGCCCGACATCGTGA |
| | | TCACCCAGGATGAGTTATCAAATCCTGTTACCTCCG |
| | | GGGAGAGCGTGTCCATCTCTTGTAGGTCCTCCAAGA |
| | | GCTTGCTTTACAAGGACGGCAAGACCTACCTGAATT |
| | | GGTTCCTGCAACGGCCAGGTCAGAGCCCGCAGCTTC |
| | | TGATCTACCTGATGAGTACTAGAGCCTCTGGCGTTTC |
| | | GGACCGCTTCTCGGGCTCGGGGTCCGGCACCGACTT |
| | | CACCCTGGAGATCTCGCGCGTTAAGGCGGAAGACGT |
| | | GGGGGTGTACTACTGTCAACAGTTAGTGGAATACCC |
| | | CTTCACCTTCGGCGGAGGAACCAAGCTGGAGGTGAA |
| | | AGGCGGCGGTGGCTCGGGGGGCGGTGGCTCCGGGG |
| | | GAGGAGGTTCCGGCGGAGGGGGTTCCGGCGTACAG |
| | | CTTCAGGAGAGCGGTGCTGAACTGGCTAGGCCCGGG |
| | | GCTTCTGTGAAGATGAGCTGCAAGGCTAGCGGCTAC |
| | | ACCTTCACCAGTTACACCATGCAGTGGGTCAAGCAG |
| | | CGCCCGGGCCAGGGCCTGGAGTGGATCGGCTACATT |
| | | AATCCCTCTTCTGGGTACATCAAGTACAACCAGAAA |
| | | TTCAAAGATAAAGTGACTCTCACTGCGGACAAGTCC |
| | | TCGACGACAGCGTATATGCAGCTGAGCCGCCTGACC |
| | | AGCGAGGACAGCGCGGTGTACTACTGCGCGCGCCGC |
| | | GGGAGCTACTTCTTCGACTTTTGGGGCCAGGGCACA |
| | | TCCGTCACCGTGTCCTCTACCACCACACCCGCGCCGC |
| | | GCCCTCCTACTCCAGCTCCCACCATTGCCTCCCAGCC |
| | | TCTTTCGCTGCGCCCAGAGGCTTGTCGGCCCGCGGC |
| | | CGGGGGAGCAGTGCACACCCGCGGCTTGGATTTCGC |
| | | TTGCGATATATATATTTGGGCGCCTCTGGCCGGCACC |
| | | TGCGGTGTACTACTGCTGTCGTTGGTGATCACCCTCT |
| | | ACTGCAAGCGTGGCCGCAAGAAGCTCCTCTACATCT |
| | | TCAAACAGCCCTTCATGCGTCCCGTCCAGACTACCC |
| | | AGGAGGAGGATGGTTGTTCTTGTCGGTTTCCCGAGG |
| | | AAGAGGAGGGCGGCTGTGAGCTCAGAGTCAAATTTT |
| | | CCCGCTCGGCCGACGCTCCGGCCTACAAGCAGGGCC |
| | | AGAACCAGCTGTATAACGAGCTCAACCTGGGACGCC |
| | | GCGAAGAGTACGATGTCCTGGACAAGCGACGAGGC |
| | | CGTGACCCGGAAATGGGCGGCAAGCCCCGCCGCAA |
| | | AAACCCTCAGGAGGGCCTGTATAACGAGCTACAGAA |
| | | GGACAAAATGGCCGAGGCCTACAGTGAGATCGGTAT |
| | | GAAGGGAGAGCGTCGCCGCGGTAAGGGCCACGACG |
| | | GATTGTATCAAGGTCTGTCTACTGCGACTAAGGACA |
| | | CGTACGACGCGCTCCATATGCAGGCCCTGCCCCCCC |
| | | GCGGTAGCGGTGCCACGAACTTTTCACTGCTCAAGC |
| | | AGGCTGGCGACGTGGAGGAGAACCCTGGACCCATG |
| | | GGGGCCGGCGCAACCGGCCGGGCCATGGATGGCCCT |
| | | CGCCTGCTGCTGTTACTGCTCCTTGGCGTGAGTCTAG |
| | | GCGGCGCCAAGGAGGCGTGCCCGACCGGCTTATACA |
| | | CCCATTCCGGGGAGTGTTGCAAGGCCTGTAACCTGG |
| | | GCGAGGGAGTGGCACAGCCATGCGGGGCCAACCAG |
| | | ACAGTGTGCGAGCCGTGCCTGGACTCAGTGACCTTT |
| | | TCCGACGTGGTGTCCGCTACAGAGCCATGCAAGCCA |
| | | TGCACTGAGTGCGTGGGGCTGCAGTCTATGAGCGCC |
| | | CCTTGCGTGGAGGCTGATGACGCCGTGTGCAGGTGT |
| | | GCCTACGGCTACTACCAGGACGAGACCACAGGCCGC |
| | | TGCGAGGCATGTCGCGTTTGCGAGGCTGGCTCCGGC |
| | | CTGGTGTTTTCCTGCCAGGACAAGCAGAACACGGTA |
| | | TGTGAAGAGTGCCCAGATGGCACGTACAGCGACGA |
| | | GGCTAATCACGTGGATCCGTGCCTGCCTTGCACTGTC |
| | | TGTGAGGACACCGAGCGCCAGCTGCGAGAGTGTACC |
| | | CGTTGGGCCGATGCTGAGTGTGAAGAGATTCCCGGC |
| | | CGGTGGATCACCCGCAGCACGCCTCCTGAGGGCTCT |
| | | GACTCCACCGCCCCCTCCACCCAGGAGCCCGAGGCC |
| | | CCGCCCGAACAGGACCTCATCGCATCTACGGTGGCA |
| | | GGTGTCGTGACTACCGTGATGGGATCTTCCCAGCCC |
| | | GTGGTCACCAGAGGTACCACGGACAACCTGATCCCG |
| | | GTGTATTGTTCGATCCTGGCTGCCGTTGTCGTCGGCC |
| | | TGGTAGCCTATATTGCGTTCAAGCGTTGGAACTCCTG |
| | | A |
| 177 | 4B2 VL-VH AA | MALPVTALLLPLALLLHAARPDIVITQDELSNPVTSGES |
| | | VSISCRSSKSLLYKDGKTYLNWFLQRPGQSPQLLIYLM |
| | | STRASGVSDRFSGSGSGTDFTLEISRVKAEDVGVYYCQ |
| | | QLVEYPFTFGGGTKLEVKGGGGSGGGGSGGGGSGGG |
| | | GSGVQLQESGAELARPGASVKMSCKASGYTFTSYTMQ |
| | | WVKQRPGQGLEWIGYINPSSGYIKYNQKFKDKVTLTA |
| | | DKSSTTAYMQLSRLTSEDSAVYYCARRGSYFFDFWGQ |
| | | GTSVTVSSTTTPAPRPPTPAPTIASQPLSLRPEACRPAAG |
| | | GAVHTRGLDFACDIYIWAPLAGTCGVLLLSLVITLYCK |
| | | RGRKKLLYIFKQPFMRPVQTTQEEDGCSCRFPEEEEGG |
| | | CELRVKFSRSADAPAYKQGQNQLYNELNLGRREEYDV |
| | | LDKRRGRDPEMGGKPRRKNPQEGLYNELQKDKMAEA |
| | | YSEIGMKGERRRGKGHDGLYQGLSTATKDTYDALHM |
| | | QALPPRGSGATNFSLLKQAGDVEENPGPMGAGATGRA |
| | | MDGPRLLLLLLLGVSLGGAKEACPTGLYTHSGECCKA |
| | | CNLGEGVAQPCGANQTVCEPCLDSVTFSDVVSATEPC |
| | | KPCTECVGLQSMSAPCVEADDAVCRCAYGYYQDETT |
| | | GRCEACRVCEAGSGLVFSCQDKQNTVCEECPDGTYSD |
| | | EANHVDPCLPCTVCEDTERQLRECTRWADAECEEIPGR |
| | | WITRSTPPEGSDSTAPSTQEPEAPPEQDLIASTVAGVVT |
| | | TVMGSSQPVVTRGTTDNLIPVYCSILAAVVVGLVAYIA |
| | | FKRWNS* |
| 178 | 9.4 VH-VL | ATGGCGCTGCCCGTGACAGCCCTTCTGCTGCCCCTG |
| | | GCGCTGCTCCTGCACGCCGCTAGGCCTCAGGTGCAG |
| | | CTCCAGCAGCTGGGAGCCGAGCTGGCCCGCCCCGGC |
| | | GCTTCCGTGAAGATGTCTTGCAAAGCATCCGGTTAC |
| | | ACTTTCACCTCGTACAGCATCCAGTGGGTGAAGCAG |
| | | CGCCCCGGACAGGGCCTGGAGTGGATCGGCTATATT |
| | | AATCCATCATCGGGGTATATCAAGTACAACCAGCAC |
| | | TTTCGTGACCGCGCCACCCTTACTGCGGACCGCTCCT |
| | | CTTCAACCGCCTACATGCAACTGAGTAGCCTGACCT |
| | | CCGAAGACAGCGCCGTGTACTACTGCGCTCGTGGGA |
| | | ATAGTGGGTCCTTCGACTATTGGGGCCAGGGAACCA |
| | | CCCTGACTGTTTCCTCTGGCGGCGGCGGTTCTGGCGG |
| | | CGGCGGTAGCGGCGGAGGTGGCTCCGGTGGGGGCG |
| | | GCTCCGACATCGTCATGACCCAGGCAGCCCCCTCCG |
| | | TGCCCGTGACCCCGGGAGAGAGCCTTAGCATCAGCT |
| | | GTCGATCCAGCAAGTCCTTGCTCCACAGCTCTGGCA |
| | | TCACGTACCTATATTGGTTCCTGCAGAGACCCGGAC |
| | | AGAGCCCACAGCTTCTCATTTACCGCATGTCTAACTT |
| | | GGCTTCTGGGGTTCCGGACAGGTTTTCGGGATCCGG |
| | | GAGCGGCACTGCGTTCACTCTGCGCATCTCCCGCGT |
| | | GGAGGCCGAAGATGTGGGAGTTTACTACTGCATGCA |
| | | GCACCTGGAGTACCCATTTACCTTCGGCGGTGGTAC |
| | | CAAGCTCGAGATTAAGACGACAACCCCGGCCCCGCG |
| | | TCCTCCTACCCCCGCCCCCACCATTGCAAGCCAGCC |
| | | ACTGTCTCTGCGCCCGGAGGCGTGCCGACCCGCTGC |
| | | AGGTGGTGCCGTGCACACTCGCGGCCTCGATTTCGC |
| | | TTGCGACATCTACATCTGGGCTCCATTGGCCGGGAC |
| | | CTGCGGGGTATTGCTGCTGTCCCTGGTGATCACTCTT |
| | | TACTGCAAGCGCGGCCGGAAAAAGCTGCTCTACATC |
| | | TTCAAGCAACCCTTCATGAGGCCTGTCCAGACTACC |
| | | CAGGAGGAAGACGGCTGTTCTTGCCGCTTCCCTGAA |
| | | GAGGAGGAAGGTGGCTGCGAGCTGCGGGTGAAATT |
| | | TTCCCGCAGTGCGGACGCCCCCGCCTACAAGCAGGG |
| | | CCAGAACCAGCTGTACAACGAGCTCAACCTGGGCCG |
| | | CCGCGAGGAGTACGACGTGCTGGACAAACGGCGCG |
| | | GCCGTGATCCGGAGATGGGTGGCAAGCCTCGTAGAA |
| | | AGAATCCTCAGGAGGGCCTGTACAACGAGCTGCAGA |
| | | AGGATAAAATGGCCGAGGCGTACTCGGAGATCGGG |
| | | ATGAAAGGCGAGCGCCGTAGGGGAAAGGGCCATGA |
| | | CGGTCTCTATCAGGGATTGTCTACCGCCACGAAGGA |
| | | CACCTATGACGCACTGCATATGCAGGCCCTCCCGCC |
| | | CCGGGGTTCCGGCGCCACCAACTTTTCACTGCTGAA |
| | | GCAGGCTGGTGACGTGGAGGAGAATCCAGGCCCCAT |
| | | GGGGGCCGGCGCCACCGGGCGCGCCATGGATGGAC |
| | | CCCGGCTGTTATTACTGCTGCTGCTTGGCGTGTCCCT |
| | | AGGCGGGGCCAAGGAGGCTTGTCCTACCGGGCTCTA |
| | | CACCCACTCGGGGGAATGTTGTAAGGCCTGTAACCT |
| | | GGGCGAGGGCGTGGCGCAGCCGTGCGGGGCGAACC |
| | | AGACGGTCTGTGAGCCATGCTTGGACAGTGTGACCT |
| | | TCTCTGATGTGGTGTCCGCCACCGAGCCTTGTAAGCC |
| | | GTGCACAGAGTGCGTGGGCCTGCAATCCATGAGCGC |
| | | GCCGTGCGTGGAGGCAGATGACGCTGTCTGCCGCTG |
| | | TGCTTACGGCTATTACCAGGATGAGACTACCGGCAG |
| | | GTGTGAGGCTTGCCGCGTATGTGAGGCGGGCTCGGG |
| | | CCTTGTATTCTCTTGCCAGGACAAGCAGAACACGGT |
| | | GTGCGAGGAGTGTCCCGACGGCACCTACAGTGACGA |
| | | GGCCAACCACGTGGACCCCTGCCTGCCGTGCACCGT |
| | | GTGCGAGGACACCGAGCGACAGTTGCGCGAGTGCA |
| | | CTCGTTGGGCTGACGCGGAATGTGAGGAGATCCCCG |
| | | GTCGCTGGATCACCCGCTCCACCCCTCCTGAAGGAT |
| | | CCGATTCGACTGCCCCCTCTACCCAGGAGCCCGAGG |
| | | CTCCCCCTGAGCAGGACCTGATTGCTTCGACTGTCGC |
| | | TGGCGTTGTCACCACAGTCATGGGCTCATCGCAGCC |
| | | GGTCGTCACCAGGGGCACGACCGACAACCTGATACC |
| | | CGTGTACTGCTCTATCCTGGCAGCCGTGGTGGTCGG |
| | | CCTGGTGGCTTACATCGCGTTCAAACGCTGGAACTC |
| | | CTGA |
| 179 | 9.4 VH-VL AA | MALPVTALLLPLALLLHAARPQVQLQQLGAELARPGA |
| | | SVKMSCKASGYTFTSYSIQWVKQRPGQGLEWIGYINPS |
| | | SGYIKYNQHFRDRATLTADRSSSTAYMQLSSLTSEDSA |
| | | VYYCARGNSGSFDYWGQGTTLTVSSGGGGSGGGGSG |
| | | GGGSGGGGSDIVMTQAAPSVPVTPGESLSISCRSSKSLL |
| | | HSSGITYLYWFLQRPGQSPQLLIYRMSNLASGVPDRES |
| | | GSGSGTAFTLRISRVEAEDVGVYYCMQHLEYPFTFGG |
| | | GTKLEIKTTTPAPRPPTPAPTIASQPLSLRPEACRPAAGG |
| | | AVHTRGLDFACDIYIWAPLAGTCGVLLLSLVITLYCKR |
| | | GRKKLLYIFKQPFMRPVQTTQEEDGCSCRFPEEEEGGC |
| | | ELRVKFSRSADAPAYKQGQNQLYNELNLGRREEYDVL |
| | | DKRRGRDPEMGGKPRRKNPQEGLYNELQKDKMAEAY |
| | | SEIGMKGERRRGKGHDGLYQGLSTATKDTYDALHMQ |
| | | ALPPRGSGATNFSLLKQAGDVEENPGPMGAGATGRA |
| | | MDGPRLLLLLLLGVSLGGAKEACPTGLYTHSGECCKA |
| | | CNLGEGVAQPCGANQTVCEPCLDSVTFSDVVSATEPC |
| | | KPCTECVGLQSMSAPCVEADDAVCRCAYGYYQDETT |
| | | GRCEACRVCEAGSGLVFSCQDKQNTVCEECPDGTYSD |
| | | EANHVDPCLPCTVCEDTERQLRECTRWADAECEEIPGR |
| | | WITRSTPPEGSDSTAPSTQEPEAPPEQDLIASTVAGVVT |
| | | TVMGSSQPVVTRGTTDNLIPVYCSILAAVVVGLVAYIA |
| | | FKRWNS* |
| 180 | 9.4 VL-VH | ATGGCTCTGCCCGTTACCGCGCTGCTCCTCCCCCTGG |
| | | CTCTGCTGCTTCACGCCGCTCGCCCGGACATCGTGAT |
| | | GACCCAGGCCGCACCCAGCGTGCCCGTGACCCCCGG |
| | | GGAGAGCCTGTCCATCTCGTGCCGATCCTCCAAGTC |
| | | CCTGTTGCACAGTTCGGGCATCACCTACCTGTACTGG |
| | | TTCCTGCAGAGGCCCGGCCAGAGCCCTCAGCTGCTC |
| | | ATTTACAGGATGTCTAACCTGGCTTCCGGTGTGCCCG |
| | | ACCGCTTCAGCGGCTCGGGCAGTGGAACCGCGTTCA |
| | | CGCTGCGCATCTCTCGCGTGGAGGCCGAGGACGTGG |
| | | GCGTGTACTACTGTATGCAGCACCTGGAGTATCCCTT |
| | | CACCTTCGGCGGCGGCACCAAGCTGGAGATCAAAGG |
| | | CGGTGGCGGCTCGGGCGGAGGCGGAAGTGGAGGCG |
| | | GCGGTTCGGGAGGGGGTGGTTCCCAGGTGCAGCTCC |
| | | AGCAGTTAGGTGCCGAGCTGGCGCGGCCTGGTGCTT |
| | | CCGTAAAAATGAGCTGTAAGGCGTCTGGGTACACGT |
| | | TCACCAGCTACAGCATCCAGTGGGTCAAGCAACGCC |
| | | CCGGACAGGGCCTTGAATGGATTGGCTACATTAATC |
| | | CCTCTTCTGGCTATATTAAGTACAACCAGCACTTTCG |
| | | TGACCGCGCTACCCTCACTGCTGATCGCTCTTCCAGC |
| | | ACCGCCTACATGCAGTTGTCTTCACTAACCTCCGAG |
| | | GACTCCGCCGTATACTACTGCGCCCGCGGCAACTCC |
| | | GGGTCCTTCGACTACTGGGGCCAGGGCACAACCCTG |
| | | ACCGTCAGCTCGACGACGACGCCTGCCCCGCGCCCC |
| | | CCCACTCCGGCTCCTACCATCGCCAGCCAGCCACTTT |
| | | CTCTGCGACCCGAGGCCTGCCGCCCCGCTGCTGGTG |
| | | GCGCTGTCCACACCCGCGGCCTCGATTTTGCATGTG |
| | | ACATCTACATCTGGGCTCCCCTGGCCGGCACCTGCG |
| | | GGGTTCTGTTACTGTCCCTGGTGATCACTCTTTATTG |
| | | CAAACGTGGCAGAAAGAAGCTGCTGTACATCTTCAA |
| | | ACAGCCGTTTATGCGTCCTGTGCAGACGACCCAGGA |
| | | GGAAGATGGGTGCTCTTGCCGTTTCCCCGAGGAGGA |
| | | GGAGGGCGGGTGCGAGCTGAGAGTGAAATTTTCTCG |
| | | CAGCGCAGACGCTCCGGCCTACAAGCAGGGTCAGA |
| | | ACCAGCTGTACAACGAGCTCAACCTCGGACGCCGGG |
| | | AGGAGTACGATGTGCTGGACAAAAGACGTGGGCGG |
| | | GATCCAGAGATGGGGGGAAAGCCCCGCAGGAAGAA |
| | | CCCTCAGGAAGGACTATACAACGAGCTGCAGAAGG |
| | | ATAAGATGGCGGAGGCCTACAGCGAAATCGGTATG |
| | | AAAGGCGAGCGGCGCCGCGGGAAGGGTCATGACGG |
| | | ACTGTATCAAGGACTGTCCACTGCTACTAAGGATAC |
| | | CTACGACGCGCTCCACATGCAGGCCCTGCCCCCGCG |
| | | GGGCTCTGGCGCGACCAACTTTAGCCTACTCAAGCA |
| | | GGCCGGGGACGTGGAGGAGAACCCGGGGCCAATGG |
| | | GGGCAGGTGCCACCGGTCGCGCTATGGATGGCCCCC |
| | | GCCTGTTGCTGTTGCTGCTGCTGGGCGTTTCGTTGGG |
| | | CGGCGCCAAGGAGGCTTGTCCCACCGGCCTTTACAC |
| | | CCATTCCGGTGAGTGTTGCAAGGCCTGCAACCTGGG |
| | | CGAGGGCGTCGCCCAGCCTTGCGGGGCCAACCAGAC |
| | | CGTGTGCGAACCTTGCCTCGACAGCGTCACCTTCTCC |
| | | GACGTGGTGTCAGCGACTGAGCCATGTAAGCCATGC |
| | | ACGGAGTGCGTGGGTCTTCAGTCCATGTCCGCCCCTT |
| | | GCGTGGAGGCCGATGACGCCGTCTGTAGGTGTGCTT |
| | | ACGGCTACTACCAGGACGAGACGACAGGTCGTTGCG |
| | | AGGCTTGTCGTGTATGCGAGGCAGGATCCGGCCTGG |
| | | TGTTCTCTTGTCAGGATAAGCAGAACACTGTCTGTG |
| | | AGGAATGCCCAGACGGCACTTACAGCGACGAGGCC |
| | | AATCACGTGGACCCGTGCCTGCCGTGCACTGTCTGT |
| | | GAAGACACCGAGCGCCAGCTGCGCGAGTGTACCCGT |
| | | TGGGCGGACGCAGAGTGCGAGGAGATCCCTGGCCG |
| | | CTGGATTACCCGCTCGACACCTCCTGAGGGCTCTGA |
| | | CAGCACAGCCCCATCCACCCAGGAACCGGAGGCGCC |
| | | CCCGGAGCAGGACCTGATAGCCTCTACCGTGGCGGG |
| | | TGTCGTCACCACCGTGATGGGTTCGTCACAGCCCGT |
| | | AGTGACTCGCGGCACCACCGACAACTTGATCCCGGT |
| | | GTATTGCTCCATCCTGGCGGCGGTGGTCGTGGGCCTT |
| | | GTGGCCTATATCGCGTTCAAGCGTTGGAATTCCTGA |
| 181 | 9.4 VL-VH AA | MALPVTALLLPLALLLHAARPDIVMTQAAPSVPVTPGE |
| | | SLSISCRSSKSLLHSSGITYLYWFLQRPGQSPQLLIYRMS |
| | | NLASGVPDRESGSGSGTAFTLRISRVEAEDVGVYYCM |
| | | QHLEYPFTFGGGTKLEIKGGGGSGGGGSGGGGSGGGG |
| | | SQVQLQQLGAELARPGASVKMSCKASGYTFTSYSIQW |
| | | VKQRPGQGLEWIGYINPSSGYIKYNQHFRDRATLTADR |
| | | SSSTAYMQLSSLTSEDSAVYYCARGNSGSFDYWGQGT |
| | | TLTVSSTTTPAPRPPTPAPTIASQPLSLRPEACRPAAGGA |
| | | VHTRGLDFACDIYIWAPLAGTCGVLLLSLVITLYCKRG |
| | | RKKLLYIFKQPFMRPVQTTQEEDGCSCRFPEEEEGGCE |
| | | LRVKFSRSADAPAYKQGQNQLYNELNLGRREEYDVLD |
| | | KRRGRDPEMGGKPRRKNPQEGLYNELQKDKMAEAYS |
| | | EIGMKGERRRGKGHDGLYQGLSTATKDTYDALHMQA |
| | | LPPRGSGATNFSLLKQAGDVEENPGPMGAGATGRAM |
| | | DGPRLLLLLLLGVSLGGAKEACPTGLYTHSGECCKAC |
| | | NLGEGVAQPCGANQTVCEPCLDSVTFSDVVSATEPCK |
| | | PCTECVGLQSMSAPCVEADDAVCRCAYGYYQDETTG |
| | | RCEACRVCEAGSGLVFSCQDKQNTVCEECPDGTYSDE |
| | | ANHVDPCLPCTVCEDTERQLRECTRWADAECEEIPGR |
| | | WITRSTPPEGSDSTAPSTQEPEAPPEQDLIASTVAGVVT |
| | | TVMGSSQPVVTRGTTDNLIPVYCSILAAVVVGLVAYIA |
| | | FKRWNS |
| 182 | 4122 VH-VL | ATGGCCCTTCCGGTGACTGCGCTGCTGTTACCACTGG |
| | | CGCTGCTTCTACACGCCGCCAGGCCCCTGTCCCTGG |
| | | AGGAATCCGGAGGGGACCTAGTGAAGCCTGGCGCTT |
| | | CCCTCACCCTGACCTGCACTGCCTCGGGCTTCTCCTT |
| | | CAGTGCAGGCTATTGGATTTGCTGGGTGCGCCAGGC |
| | | TCCCGGCAAAGGCCTGGAGTGGATTGCGTGCACTTA |
| | | CGCGGGCCGCAGTGGATCGACCTACTATGCCAACTG |
| | | GGTCAACGGGCGTTTCACCATCCCGAAGACCTCCAG |
| | | CACGACCGTCACCCTTCAGATGACCAGCTTATCAGG |
| | | CGCTGACACCGCTAGCTATTTCTGCGCCCGGGGCAA |
| | | CGCGGGTGTAGCGGTGGGTGCTCTTTGGGGCCCTGG |
| | | TACATTGGTTACTGTCAGCTCTGGCGGGGGAGGCTC |
| | | TGGCGGCGGTGGTAGCGGGGGCGGAGGCTCTGGCG |
| | | GTGGGGGTTCCGACATCGTGATGACCCAGACCCCTG |
| | | CCTCCGTTTCGGAACCAGTCGGCGGAACCGTCACTA |
| | | TCATGTGCCAGGCGTCCCAGAGCATCTCAAATTGGC |
| | | TAGCCTGGTACCAGCAGAAGCCCGGACAGCCCCCCA |
| | | AGCTGTTAATCTACCAGGCATCCAAGCTGGCCTCCG |
| | | GAGTCCCGTCGCGCTTTAAGGGTTCTGGCTCTGGGA |
| | | CTGAGTACACCCTGACCATTTCTGACCTCGAGTGTGC |
| | | CGATGCGGCTACATATTACTGTCAAAGCTACTACGA |
| | | TAGTGGCAGCAACGTGTTTTTTGCTTTCGGGGGAGG |
| | | TACCAAGGTGGTCGTGGAGACTACCACCCCGGCACC |
| | | CCGGCCGCCTACCCCAGCGCCCACCATCGCTTCTCA |
| | | ACCCCTGTCTCTGCGCCCCGAGGCCTGCCGTCCGGCT |
| | | GCTGGTGGCGCCGTACACACCCGCGGTTTGGACTTC |
| | | GCATGCGACATCTACATATGGGCCCCCTTGGCCGGC |
| | | ACCTGCGGGGTGTTACTGCTCAGCCTGGTGATTACA |
| | | CTGTATTGTAAGCGCGGCCGCAAGAAGCTCTTGTAC |
| | | ATCTTCAAACAGCCGTTCATGCGCCCTGTGCAGACG |
| | | ACCCAGGAGGAAGACGGGTGTTCTTGCCGCTTCCCC |
| | | GAGGAGGAGGAGGGTGGGTGCGAGCTGCGCGTGAA |
| | | ATTTTCCAGGAGCGCGGACGCACCCGCGTACAAGCA |
| | | GGGCCAGAACCAGTTGTACAACGAGCTCAACCTGGG |
| | | CAGACGGGAGGAGTACGACGTGCTGGACAAGCGCC |
| | | GTGGACGCGATCCAGAGATGGGCGGTAAGCCGCGA |
| | | CGCAAGAACCCTCAGGAGGGCCTGTACAACGAGCTG |
| | | CAGAAGGACAAAATGGCGGAGGCATACTCCGAGAT |
| | | CGGTATGAAGGGCGAGCGGCGCCGAGGCAAGGGGC |
| | | ATGACGGCCTCTACCAGGGCCTGTCAACTGCCACTA |
| | | AGGACACCTACGACGCTCTGCACATGCAGGCCCTTC |
| | | CCCCGCGCGGCTCCGGTGCCACCAACTTTTCTTTGCT |
| | | GAAGCAGGCTGGGGATGTCGAGGAAAACCCTGGCC |
| | | CCATGGGCGCTGGAGCAACCGGCAGAGCCATGGAT |
| | | GGGCCCCGCCTGCTGCTGCTGTTGCTGCTGGGGGTA |
| | | TCGCTTGGTGGCGCCAAGGAGGCCTGCCCAACCGGT |
| | | CTGTACACGCACTCCGGGGAGTGCTGTAAAGCGTGT |
| | | AATCTGGGCGAGGGCGTAGCTCAGCCGTGCGGGGCT |
| | | AACCAGACTGTTTGTGAACCCTGTCTGGACTCGGTG |
| | | ACCTTCTCGGATGTGGTTTCCGCCACGGAGCCATGC |
| | | AAACCTTGTACGGAGTGCGTGGGACTCCAGTCCATG |
| | | TCCGCACCGTGCGTGGAGGCCGATGACGCCGTGTGC |
| | | CGATGCGCGTACGGCTACTACCAAGACGAGACCACT |
| | | GGCAGGTGTGAGGCCTGTCGCGTCTGTGAGGCTGGA |
| | | TCGGGCCTCGTGTTCTCGTGTCAGGATAAACAGAAC |
| | | ACGGTGTGCGAGGAGTGCCCCGACGGCACCTATTCC |
| | | GACGAAGCCAATCATGTGGACCCGTGTCTGCCCTGC |
| | | ACTGTCTGCGAGGACACCGAGCGCCAGCTGCGGGAG |
| | | TGTACCCGCTGGGCGGATGCCGAGTGCGAGGAGATC |
| | | CCTGGGCGTTGGATCACCAGGAGCACACCTCCTGAG |
| | | GGCTCCGACAGCACGGCGCCCTCTACCCAGGAGCCT |
| | | GAAGCGCCCCCGGAACAGGACTTAATCGCCAGCACC |
| | | GTGGCCGGCGTCGTCACCACTGTGATGGGCAGTTCC |
| | | CAGCCGGTGGTTACCCGTGGCACAACCGACAACCTG |
| | | ATTCCAGTGTACTGCTCTATCCTGGCTGCCGTAGTGG |
| | | TGGGCCTGGTGGCTTACATCGCGTTCAAGCGTTGGA |
| | | ATTCCTGA |
| 183 | 4122 VH-VL AA | MALPVTALLLPLALLLHAARPLSLEESGGDLVKPGASL |
| | | TLTCTASGFSFSAGYWICWVRQAPGKGLEWIACTYAG |
| | | RSGSTYYANWVNGRFTIPKTSSTTVTLQMTSLSGADTA |
| | | SYFCARGNAGVAVGALWGPGTLVTVSSGGGGSGGGG |
| | | SGGGGSGGGGSDIVMTQTPASVSEPVGGTVTIMCQAS |
| | | QSISNWLAWYQQKPGQPPKLLIYQASKLASGVPSRFKG |
| | | SGSGTEYTLTISDLECADAATYYCQSYYDSGSNVFFAF |
| | | GGGTKVVVETTTPAPRPPTPAPTIASQPLSLRPEACRPA |
| | | AGGAVHTRGLDFACDIYIWAPLAGTCGVLLLSLVITLY |
| | | CKRGRKKLLYIFKQPFMRPVQTTQEEDGCSCRFPEEEE |
| | | GGCELRVKFSRSADAPAYKQGQNQLYNELNLGRREEY |
| | | DVLDKRRGRDPEMGGKPRRKNPQEGLYNELQKDKMA |
| | | EAYSEIGMKGERRRGKGHDGLYQGLSTATKDTYDAL |
| | | HMQALPPRGSGATNFSLLKQAGDVEENPGPMGAGAT |
| | | GRAMDGPRLLLLLLLGVSLGGAKEACPTGLYTHSGEC |
| | | CKACNLGEGVAQPCGANQTVCEPCLDSVTFSDVVSAT |
| | | EPCKPCTECVGLQSMSAPCVEADDAVCRCAYGYYQD |
| | | ETTGRCEACRVCEAGSGLVFSCQDKQNTVCEECPDGT |
| | | YSDEANHVDPCLPCTVCEDTERQLRECTRWADAECEE |
| | | IPGRWITRSTPPEGSDSTAPSTQEPEAPPEQDLIASTVAG |
| | | VVTTVMGSSQPVVTRGTTDNLIPVYCSILAAVVVGLV |
| | | AYIAFKRWNS* |
| 184 | 4122 VL-VH | ATGGCTCTGCCTGTGACCGCACTGCTGCTCCCGCTGG |
| | | CGCTGTTGCTCCACGCCGCTCGGCCCGACATCGTGA |
| | | TGACCCAGACACCAGCGAGTGTGAGCGAGCCAGTG |
| | | GGTGGCACAGTCACTATCATGTGCCAGGCTTCACAG |
| | | TCCATCTCAAATTGGCTCGCTTGGTACCAGCAGAAG |
| | | CCGGGACAGCCGCCCAAGCTGCTGATTTATCAGGCT |
| | | TCCAAGCTGGCCTCCGGTGTGCCAAGCCGCTTCAAA |
| | | GGCAGCGGGTCTGGTACTGAGTACACCCTGACCATC |
| | | AGTGACCTGGAGTGCGCGGACGCCGCTACCTATTAC |
| | | TGTCAGAGCTACTACGACAGCGGCAGCAACGTGTTC |
| | | TTTGCCTTCGGCGGGGGAACCAAGGTGGTGGTGGAG |
| | | GGCGGCGGGGGATCCGGTGGTGGCGGCTCCGGCGG |
| | | CGGCGGCTCCGGTGGAGGAGGAAGTTTGTCTCTTGA |
| | | GGAGAGCGGGGGGGACCTGGTGAAGCCCGGCGCCT |
| | | CCCTGACCCTGACCTGTACGGCTTCGGGCTTCTCCTT |
| | | CTCGGCTGGGTATTGGATTTGTTGGGTGCGGCAGGC |
| | | CCCAGGCAAAGGCTTGGAGTGGATCGCCTGCACATA |
| | | TGCGGGCCGCAGCGGCTCGACTTACTACGCCAACTG |
| | | GGTCAACGGCCGCTTCACCATCCCGAAGACCAGCTC |
| | | CACGACCGTCACTCTTCAGATGACCTCTCTGTCCGGT |
| | | GCGGACACCGCTTCGTATTTCTGTGCACGCGGTAAC |
| | | GCGGGCGTGGCGGTGGGCGCGCTTTGGGGCCCCGGC |
| | | ACTCTGGTTACCGTGTCCTCCACCACTACTCCCGCCC |
| | | CGCGCCCTCCTACCCCGGCCCCCACCATTGCTAGTCA |
| | | GCCACTTAGCCTGAGGCCCGAGGCGTGCCGACCTGC |
| | | CGCCGGCGGCGCCGTCCACACTCGCGGTTTGGACTT |
| | | TGCCTGTGACATCTACATATGGGCGCCCCTGGCCGG |
| | | GACCTGCGGGGTGCTGCTGCTTTCCCTCGTGATTACG |
| | | CTGTACTGTAAACGCGGTCGCAAGAAGCTGTTATAT |
| | | ATCTTCAAGCAGCCCTTCATGCGCCCCGTGCAGACT |
| | | ACCCAGGAGGAGGACGGGTGCTCATGCCGCTTTCCT |
| | | GAGGAAGAGGAGGGCGGGTGCGAGCTGCGGGTAAA |
| | | ATTTTCCAGAAGTGCGGACGCACCTGCCTACAAGCA |
| | | AGGCCAGAACCAGCTCTACAACGAGCTCAACCTGGG |
| | | CCGCAGAGAGGAGTACGATGTCCTTGACAAGCGTCG |
| | | TGGCCGGGACCCGGAGATGGGCGGCAAGCCGCGCA |
| | | GAAAGAACCCTCAGGAAGGCCTTTACAACGAGCTGC |
| | | AGAAGGACAAAATGGCCGAGGCTTACTCTGAGATCG |
| | | GTATGAAGGGAGAGCGCCGCCGCGGGAAGGGTCAC |
| | | GATGGACTGTACCAGGGCCTGTCCACCGCTACTAAG |
| | | GACACCTACGACGCCCTCCATATGCAGGCCTTGCCC |
| | | CCGCGTGGTTCTGGCGCCACCAACTTTTCCCTACTGA |
| | | AACAGGCCGGGGATGTGGAGGAGAACCCAGGCCCT |
| | | ATGGGCGCTGGTGCGACCGGCCGAGCCATGGATGGA |
| | | CCCAGGCTACTGCTGCTATTGCTGCTGGGCGTGTCCC |
| | | TAGGCGGAGCCAAGGAGGCCTGCCCAACCGGCCTCT |
| | | ACACGCATTCTGGAGAGTGTTGCAAGGCCTGCAACC |
| | | TGGGCGAGGGCGTGGCACAGCCTTGTGGCGCCAATC |
| | | AAACTGTATGCGAGCCATGCCTGGATTCCGTGACCT |
| | | TCTCTGATGTTGTCAGTGCTACCGAGCCCTGTAAGCC |
| | | CTGCACGGAGTGCGTGGGGTTGCAGTCTATGTCTGC |
| | | GCCGTGCGTGGAGGCCGATGACGCTGTCTGTAGGTG |
| | | TGCCTACGGCTACTACCAGGATGAGACCACCGGCCG |
| | | ATGCGAGGCTTGCAGGGTCTGCGAGGCTGGTTCTGG |
| | | GCTGGTGTTCTCCTGTCAGGACAAACAGAACACGGT |
| | | CTGTGAAGAATGCCCAGACGGCACCTACTCCGACGA |
| | | GGCCAATCACGTGGACCCCTGCCTGCCCTGCACTGT |
| | | CTGTGAAGACACGGAGCGTCAGCTCCGCGAGTGCAC |
| | | TCGCTGGGCTGACGCAGAATGTGAGGAGATCCCTGG |
| | | GCGTTGGATCACGCGCTCCACACCGCCTGAGGGCAG |
| | | CGATTCTACCGCCCCCTCTACCCAGGAGCCCGAAGC |
| | | GCCTCCTGAACAGGACCTGATCGCCTCCACCGTGGC |
| | | TGGAGTTGTCACAACCGTGATGGGCAGCTCCCAACC |
| | | GGTGGTGACCCGCGGAACAACCGACAACCTGATTCC |
| | | CGTGTACTGCTCCATCCTGGCGGCAGTGGTTGTAGG |
| | | CTTAGTCGCATACATCGCGTTCAAGCGTTGGAACTC |
| | | CTGA |
| 185 | 4122 VL-VH AA | MALPVTALLLPLALLLHAARPDIVMTQTPASVSEPVGG |
| | | TVTIMCQASQSISNWLAWYQQKPGQPPKLLIYQASKL |
| | | ASGVPSRFKGSGSGTEYTLTISDLECADAATYYCQSYY |
| | | DSGSNVFFAFGGGTKVVVEGGGGSGGGGSGGGGSGG |
| | | GGSLSLEESGGDLVKPGASLTLTCTASGFSFSAGYWIC |
| | | WVRQAPGKGLEWIACTYAGRSGSTYYANWVNGRFTI |
| | | PKTSSTTVTLQMTSLSGADTASYFCARGNAGVAVGAL |
| | | WGPGTLVTVSSTTTPAPRPPTPAPTIASQPLSLRPEACRP |
| | | AAGGAVHTRGLDFACDIYIWAPLAGTCGVLLLSLVITL |
| | | YCKRGRKKLLYIFKQPFMRPVQTTQEEDGCSCRFPEEE |
| | | EGGCELRVKFSRSADAPAYKQGQNQLYNELNLGRREE |
| | | YDVLDKRRGRDPEMGGKPRRKNPQEGLYNELQKDKM |
| | | AEAYSEIGMKGERRRGKGHDGLYQGLSTATKDTYDA |
| | | LHMQALPPRGSGATNFSLLKQAGDVEENPGPMGAGAT |
| | | GRAMDGPRLLLLLLLGVSLGGAKEACPTGLYTHSGEC |
| | | CKACNLGEGVAQPCGANQTVCEPCLDSVTFSDVVSAT |
| | | EPCKPCTECVGLQSMSAPCVEADDAVCRCAYGYYQD |
| | | ETTGRCEACRVCEAGSGLVFSCQDKQNTVCEECPDGT |
| | | YSDEANHVDPCLPCTVCEDTERQLRECTRWADAECEE |
| | | IPGRWITRSTPPEGSDSTAPSTQEPEAPPEQDLIASTVAG |
| | | VVTTVMGSSQPVVTRGTTDNLIPVYCSILAAVVVGLV |
| | | AYIAFKRWNS* |
| 186 | BC8 VH-VL | ATGGCCCTGCCAGTCACCGCCCTCCTTCTGCCCCTGG |
| | | CGCTCCTTCTGCACGCCGCTCGCCCTCAGGTCCAGTT |
| | | GGTGGAGAGCGGTGGCGGGCTGGTGCAGCCCGGGG |
| | | GGAGCCTAAAGCTCAGCTGTGCGGCTTCTGGTTTCG |
| | | ACTTTTCCCGCTATTGGATGTCTTGGGTCCGGCAGGC |
| | | CCCGGGCAAAGGCCTGGAGTGGATTGGAGAGATTA |
| | | ATCCAACCTCCAGCACTATTAATTTCACCCCCTCTCT |
| | | TAAGGATAAAGTGTTCATCTCACGAGATAACGCGAA |
| | | GAACACGCTGTACCTGCAGATGTCTAAGGTGCGCTC |
| | | GGAGGACACCGCCCTGTACTACTGCGCCCGCGGCAA |
| | | CTACTACCGCTACGGCGATGCCATGGACTATTGGGG |
| | | CCAGGGGACCAGCGTGACAGTCTCGGGCGGTGGCG |
| | | GCTCCGGGGGCGGTGGCTCCGGGGGCGGAGGTTCGG |
| | | GCGGCGGTGGCTCTGACATCGTTCTGACCCAGTCAC |
| | | CAGCGTCCCTGGCCGTATCCCTGGGGCAGCGCGCCA |
| | | CCATCTCATGTAGGGCTTCCAAGTCTGTTTCGACCTC |
| | | CGGCTATAGCTACCTACATTGGTACCAGCAGAAGCC |
| | | TGGACAGCCGCCCAAGCTGCTGATTTACCTGGCCAG |
| | | CAACTTAGAGAGTGGAGTACCAGCTCGCTTCAGCGG |
| | | CTCGGGCAGCGGTACAGACTTCACCCTCAACATCCA |
| | | CCCCGTCGAAGAGGAAGACGCTGCCACTTACTACTG |
| | | TCAACACTCACGAGAGCTCCCCTTCACCTTCGGCTCT |
| | | GGTACCAAGCTGGAGATCAAGACCACAACTCCTGCT |
| | | CCTCGCCCTCCGACCCCGGCTCCTACCATCGCTAGCC |
| | | AGCCACTATCCCTTCGGCCCGAAGCGTGTAGGCCCG |
| | | CAGCCGGCGGTGCGGTCCACACGCGGGGTTTGGACT |
| | | TCGCATGTGACATCTACATCTGGGCTCCTCTAGCGG |
| | | GTACCTGCGGAGTGCTGTTGCTGTCCCTCGTGATCAC |
| | | CCTTTACTGCAAACGAGGCCGGAAGAAGCTACTGTA |
| | | CATCTTCAAACAGCCTTTTATGCGCCCGGTGCAGAC |
| | | GACCCAGGAGGAGGACGGCTGTTCTTGCCGCTTTCC |
| | | CGAAGAGGAGGAGGGCGGGTGCGAGCTGCGCGTGA |
| | | AATTTTCGCGCAGTGCGGACGCCCCGGCCTACAAGC |
| | | AGGGTCAGAACCAGCTGTACAACGAGCTGAACCTGG |
| | | GACGCCGTGAGGAGTACGACGTGCTGGACAAGCGC |
| | | CGTGGGCGGGACCCGGAGATGGGCGGTAAGCCGAG |
| | | ACGCAAAAACCCTCAAGAGGGGCTCTATAACGAGTT |
| | | GCAGAAGGACAAAATGGCTGAGGCTTACAGTGAGA |
| | | TCGGAATGAAGGGGGAGCGCCGCCGCGGCAAGGGC |
| | | CACGATGGCCTCTATCAGGGACTGTCCACCGCGACT |
| | | AAGGACACCTACGATGCTCTGCATATGCAGGCACTT |
| | | CCACCGCGCGGCTCCGGAGCCACCAACTTTTCCCTG |
| | | CTGAAGCAAGCGGGCGATGTGGAGGAGAACCCCGG |
| | | GCCCATGGGTGCCGGCGCCACAGGCCGTGCCATGGA |
| | | TGGACCCCGTCTGCTTCTCCTGCTTTTGTTGGGGGTC |
| | | TCCTTGGGAGGCGCTAAGGAGGCATGCCCAACGGGT |
| | | CTTTACACCCACTCCGGCGAATGTTGCAAGGCCTGC |
| | | AACCTGGGCGAGGGCGTGGCGCAGCCGTGCGGGGC |
| | | CAACCAGACCGTGTGCGAGCCCTGCCTGGATTCCGT |
| | | AACTTTCTCTGACGTGGTCTCCGCTACCGAGCCTTGT |
| | | AAGCCCTGCACAGAGTGCGTGGGCCTGCAGTCTATG |
| | | TCCGCTCCTTGCGTGGAGGCAGATGACGCAGTGTGC |
| | | CGCTGTGCCTACGGCTACTATCAAGACGAGACTACC |
| | | GGCAGATGTGAGGCGTGCAGAGTTTGTGAAGCAGGC |
| | | AGCGGTCTCGTCTTCTCGTGTCAGGACAAGCAGAAC |
| | | ACTGTATGCGAGGAGTGTCCCGACGGCACCTACTCC |
| | | GACGAGGCGAATCACGTGGATCCATGCCTGCCCTGC |
| | | ACTGTCTGCGAGGACACGGAGCGCCAGCTGCGCGAG |
| | | TGTACGAGATGGGCAGACGCCGAGTGCGAGGAGAT |
| | | ACCAGGTAGGTGGATTACTCGCTCCACCCCGCCCGA |
| | | AGGCAGTGACTCCACCGCTCCTAGCACCCAGGAGCC |
| | | GGAGGCGCCTCCGGAACAGGACCTGATCGCCAGCAC |
| | | GGTGGCCGGCGTGGTTACAACCGTGATGGGATCTTC |
| | | CCAGCCCGTGGTGACCCGCGGGACCACTGACAACCT |
| | | GATCCCCGTGTACTGCTCTATCCTGGCCGCTGTGGTG |
| | | GTGGGCCTGGTGGCCTATATCGCGTTCAAGCGTTGG |
| | | AATTCCTGA |
| 187 | BC8 VH-VL AA | MALPVTALLLPLALLLHAARPQVQLVESGGGLVQPGG |
| | | SLKLSCAASGFDFSRYWMSWVRQAPGKGLEWIGEINP |
| | | TSSTINFTPSLKDKVFISRDNAKNTLYLQMSKVRSEDTA |
| | | LYYCARGNYYRYGDAMDYWGQGTSVTVSGGGGSGG |
| | | GGSGGGGSGGGGSDIVLTQSPASLAVSLGQRATISCRA |
| | | SKSVSTSGYSYLHWYQQKPGQPPKLLIYLASNLESGVP |
| | | ARFSGSGSGTDFTLNIHPVEEEDAATYYCQHSRELPFTF |
| | | GSGTKLEIKTTTPAPRPPTPAPTIASQPLSLRPEACRPAA |
| | | GGAVHTRGLDFACDIYIWAPLAGTCGVLLLSLVITLYC |
| | | KRGRKKLLYIFKQPFMRPVQTTQEEDGCSCRFPEEEEG |
| | | GCELRVKFSRSADAPAYKQGQNQLYNELNLGRREEYD |
| | | VLDKRRGRDPEMGGKPRRKNPQEGLYNELQKDKMAE |
| | | AYSEIGMKGERRRGKGHDGLYQGLSTATKDTYDALH |
| | | MQALPPRGSGATNFSLLKQAGDVEENPGPMGAGATG |
| | | RAMDGPRLLLLLLLGVSLGGAKEACPTGLYTHSGECC |
| | | KACNLGEGVAQPCGANQTVCEPCLDSVTFSDVVSATE |
| | | PCKPCTECVGLQSMSAPCVEADDAVCRCAYGYYQDE |
| | | TTGRCEACRVCEAGSGLVFSCQDKQNTVCEECPDGTY |
| | | SDEANHVDPCLPCTVCEDTERQLRECTRWADAECEEIP |
| | | GRWITRSTPPEGSDSTAPSTQEPEAPPEQDLIASTVAGV |
| | | VTTVMGSSQPVVTRGTTDNLIPVYCSILAAVVVGLVA |
| | | YIAFKRWNS* |
| 188 | CN1 VH-VL | ATGGCTCTCCCCGTCACAGCTCTGCTGCTGCCCCTGG |
| | | CCCTCCTCCTTCACGCTGCCCGCCCCCAGGTCCAGCT |
| | | GGTGCAGAGCGGAGCCGAGGTGAAGAAGCCTGGGG |
| | | CTTCTGTGAAGGTGTCGTGTAAAGCTTCCGGCTACA |
| | | CGTTCTCTGGTTACGCAATGCACTGGGTGCGCCAGG |
| | | CTCCTGGTCAGGGACTGGAGTGGATGGGGTGGATAA |
| | | ACCCCAAGAGCCCCGGCACCAACTACGCGCAGAAGT |
| | | TCCAGGGCCGAGTCACTATGACCCGCGACACGAGCA |
| | | TCAGCACGGCCTACATGGAACTGAGCCGCCTGCGCT |
| | | CCGATGACACTGCAGTGTATTACTGCGCCCGCTACT |
| | | CGAGTTCCAGTTGGGGCGACCCGTGGGGACAGGGTA |
| | | CACTGGTGACCGTGTCTTCCGGTGGGGGCGGAAGCG |
| | | GTGGTGGTGGCAGTGGAGGCGGAGGCTCCGGAGGC |
| | | GGTGGCTCGAGCTACGAGTTGACCCAGCCGCCATCC |
| | | GTGAGCGTGAGCCCCGGCCAGACTGCCAGAGTCACC |
| | | TGTTCTGGGGACGCGCAGCCCAAGGCCTACGCTTAT |
| | | TGGTACCAGCAGAAGTCTGGACAGGCCCCCATCTTG |
| | | GTGATCTACGGTGATTCCAAGCGCCCCTCTGGCATC |
| | | CCGGAGCGTTTTAGTGGGTCCTCCTCAGGCACAATG |
| | | GCGACACTGACCATTTCTGGGGCACAGGTGGAGGAT |
| | | GAAGCGGACTATTACTGTTATTCTACCGACCGCTCG |
| | | GGCAACCGCGTGTTCGGCGGGGGTACCCGCCTGACT |
| | | GTGCTGACTACCACCCCCGCCCCCCGCCCCCCTACTC |
| | | CGGCCCCAACCATCGCATCCCAGCCGCTGAGTCTGA |
| | | GGCCAGAGGCTTGTCGGCCTGCCGCTGGCGGCGCTG |
| | | TACACACCCGAGGATTGGATTTCGCGTGCGACATCT |
| | | ATATTTGGGCTCCTCTAGCTGGCACCTGTGGCGTGCT |
| | | GTTGCTCTCCCTGGTAATCACCCTGTACTGTAAGCGT |
| | | GGCCGCAAGAAGCTGCTATACATCTTCAAACAACCG |
| | | TTTATGCGCCCGGTGCAAACAACCCAGGAGGAGGAC |
| | | GGGTGCTCATGCCGCTTCCCAGAAGAAGAGGAGGGC |
| | | GGGTGCGAGCTACGCGTCAAATTTTCACGGTCTGCG |
| | | GACGCCCCTGCCTACAAGCAGGGCCAGAACCAGCTT |
| | | TACAACGAGCTCAACCTTGGTCGTCGCGAGGAGTAC |
| | | GATGTGCTCGACAAGCGAAGAGGGCGGGACCCGGA |
| | | GATGGGCGGCAAGCCACGTCGTAAGAACCCGCAGG |
| | | AGGGCCTGTACAACGAGCTGCAGAAGGACAAGATG |
| | | GCCGAGGCCTACTCCGAGATCGGCATGAAAGGCGA |
| | | GCGCCGCCGCGGAAAGGGCCACGACGGCCTCTACCA |
| | | GGGTCTCAGCACTGCGACCAAAGACACCTACGACGC |
| | | TCTGCATATGCAGGCCTTGCCCCCGCGTGGCTCCGGT |
| | | GCGACTAACTTCTCGCTCCTTAAGCAGGCCGGCGAC |
| | | GTGGAGGAGAATCCTGGGCCTATGGGTGCTGGTGCC |
| | | ACCGGCCGCGCCATGGATGGCCCCCGTCTGCTGCTA |
| | | TTGCTGTTGCTGGGCGTGTCCCTGGGTGGCGCCAAG |
| | | GAGGCCTGCCCAACCGGACTGTACACCCACTCTGGA |
| | | GAGTGTTGCAAGGCGTGTAACCTGGGCGAGGGCGTC |
| | | GCTCAGCCCTGCGGCGCCAACCAGACGGTCTGCGAG |
| | | CCCTGCCTGGACTCGGTGACCTTCTCCGACGTGGTAT |
| | | CCGCAACGGAGCCGTGCAAGCCCTGCACCGAGTGCG |
| | | TGGGCTTGCAGTCCATGTCGGCCCCTTGCGTTGAAG |
| | | CCGACGACGCAGTTTGTAGGTGTGCCTACGGGTATT |
| | | ACCAGGACGAGACTACGGGCAGATGCGAGGCGTGC |
| | | AGGGTTTGTGAGGCCGGCAGCGGTCTGGTATTTAGC |
| | | TGTCAGGACAAACAGAACACGGTCTGTGAGGAGTGC |
| | | CCAGACGGCACCTACTCCGACGAGGCCAATCATGTG |
| | | GATCCATGCCTGCCTTGCACTGTCTGTGAAGATACTG |
| | | AGCGGCAACTGAGGGAGTGCACCCGGTGGGCGGAC |
| | | GCAGAGTGCGAGGAGATTCCCGGGCGCTGGATTACC |
| | | CGCTCTACCCCTCCGGAGGGATCCGATTCTACTGCG |
| | | CCCTCCACCCAGGAGCCAGAGGCTCCTCCCGAACAG |
| | | GATCTTATCGCCAGCACCGTGGCGGGCGTGGTGACA |
| | | ACCGTGATGGGCTCGTCGCAACCCGTCGTCACTCGC |
| | | GGCACCACCGACAACCTGATCCCCGTGTACTGTTCA |
| | | ATCCTGGCTGCGGTGGTTGTCGGCTTAGTGGCTTATA |
| | | TCGCGTTCAAACGGTGGAATTCCTGA |
| 189 | CN1 VH-VL AA | MALPVTALLLPLALLLHAARPQVQLVQSGAEVKKPGA |
| | | SVKVSCKASGYTFSGYAMHWVRQAPGQGLEWMGWI |
| | | NPKSPGTNYAQKFQGRVTMTRDTSISTAYMELSRLRSD |
| | | DTAVYYCARYSSSSWGDPWGQGTLVTVSSGGGGSGG |
| | | GGSGGGGSGGGGSSYELTQPPSVSVSPGQTARVTCSGD |
| | | AQPKAYAYWYQQKSGQAPILVIYGDSKRPSGIPERFSG |
| | | SSSGTMATLTISGAQVEDEADYYCYSTDRSGNRVFGG |
| | | GTRLTVLTTTPAPRPPTPAPTIASQPLSLRPEACRPAAG |
| | | GAVHTRGLDFACDIYIWAPLAGTCGVLLLSLVITLYCK |
| | | RGRKKLLYIFKQPFMRPVQTTQEEDGCSCRFPEEEEGG |
| | | CELRVKFSRSADAPAYKQGQNQLYNELNLGRREEYDV |
| | | LDKRRGRDPEMGGKPRRKNPQEGLYNELQKDKMAEA |
| | | YSEIGMKGERRRGKGHDGLYQGLSTATKDTYDALHM |
| | | QALPPRGSGATNFSLLKQAGDVEENPGPMGAGATGRA |
| | | MDGPRLLLLLLLGVSLGGAKEACPTGLYTHSGECCKA |
| | | CNLGEGVAQPCGANQTVCEPCLDSVTFSDVVSATEPC |
| | | KPCTECVGLQSMSAPCVEADDAVCRCAYGYYQDETT |
| | | GRCEACRVCEAGSGLVFSCQDKQNTVCEECPDGTYSD |
| | | EANHVDPCLPCTVCEDTERQLRECTRWADAECEEIPGR |
| | | WITRSTPPEGSDSTAPSTQEPEAPPEQDLIASTVAGVVT |
| | | TVMGSSQPVVTRGTTDNLIPVYCSILAAVVVGLVAYIA |
| | | FKRWNS* |
| 190 | CN1 VL-VH | ATGGCGCTGCCGGTAACCGCGCTACTCCTACCCCTG |
| | | GCACTGTTGTTGCACGCCGCACGACCTAGCTACGAG |
| | | CTGACGCAGCCGCCCTCCGTGTCCGTCTCCCCTGGAC |
| | | AGACGGCCCGGGTCACCTGCAGTGGCGACGCACAGC |
| | | CCAAGGCCTATGCGTATTGGTACCAGCAGAAGTCGG |
| | | GACAGGCCCCCATCCTGGTGATTTACGGTGACTCCA |
| | | AGAGACCGTCCGGTATCCCAGAGAGGTTTTCAGGTA |
| | | GCTCCTCCGGGACCATGGCTACCCTGACCATCTCCG |
| | | GCGCTCAAGTCGAGGACGAGGCGGACTACTACTGTT |
| | | ATTCGACCGACCGCTCTGGCAACCGCGTGTTCGGCG |
| | | GGGGCACGCGACTCACTGTTCTTGGCGGAGGTGGTT |
| | | CCGGCGGAGGAGGATCGGGCGGCGGCGGTTCTGGC |
| | | GGCGGGGGCTCACAGGTGCAGCTCGTTCAGAGCGGC |
| | | GCTGAGGTGAAGAAGCCCGGCGCCAGTGTCAAGGTT |
| | | TCGTGCAAGGCCTCTGGCTACACCTTTTCCGGTTACG |
| | | CCATGCATTGGGTGCGCCAGGCCCCCGGGCAAGGCC |
| | | TGGAGTGGATGGGTTGGATTAATCCCAAATCCCCCG |
| | | GAACCAACTACGCGCAGAAGTTCCAGGGACGCGTCA |
| | | CCATGACGCGCGACACCAGCATCTCCACAGCGTACA |
| | | TGGAGCTGTCTCGCCTGCGCTCCGACGACACTGCGG |
| | | TGTACTACTGTGCCCGCTACAGCTCATCTAGCTGGG |
| | | GCGACCCGTGGGGCCAGGGCACTCTGGTGACCGTGA |
| | | GCTCCACCACTACCCCTGCGCCCAGACCGCCTACCC |
| | | CCGCCCCAACCATTGCTTCCCAGCCATTGTCTCTGCG |
| | | CCCTGAAGCGTGCCGTCCGGCTGCCGGTGGAGCGGT |
| | | GCACACCCGCGGACTCGACTTCGCTTGTGACATCTA |
| | | CATCTGGGCTCCCCTTGCTGGGACCTGTGGAGTGCT |
| | | GTTACTTTCTCTCGTGATAACACTGTATTGTAAACGT |
| | | GGCCGTAAGAAGTTGCTGTATATCTTCAAACAGCCC |
| | | TTCATGCGCCCCGTGCAGACCACCCAGGAAGAGGAT |
| | | GGGTGCTCTTGTCGCTTCCCGGAGGAGGAGGAGGGC |
| | | GGGTGTGAGCTGCGCGTCAAATTTTCCAGGTCCGCC |
| | | GACGCCCCGGCCTACAAGCAGGGTCAGAACCAGCTG |
| | | TACAACGAGCTCAACCTGGGCCGCCGCGAGGAGTAC |
| | | GACGTGCTGGACAAGCGTCGCGGTCGGGACCCTGAG |
| | | ATGGGGGGCAAGCCGCGCCGCAAGAACCCTCAGGA |
| | | GGGCCTGTACAACGAGCTGCAGAAGGACAAAATGG |
| | | CCGAGGCCTACTCCGAGATTGGAATGAAGGGAGAG |
| | | CGCCGGCGTGGCAAGGGCCATGACGGCCTTTATCAG |
| | | GGCCTGAGCACGGCCACCAAGGACACCTACGATGCC |
| | | CTCCACATGCAGGCGTTACCTCCCCGAGGGAGCGGT |
| | | GCTACCAATTTCAGTCTGCTGAAGCAGGCCGGCGAT |
| | | GTGGAGGAGAACCCGGGCCCTATGGGCGCCGGGGC |
| | | TACTGGGCGTGCTATGGATGGACCCCGCTTGCTGCT |
| | | ATTGCTGCTGCTGGGGGTGTCGTTAGGCGGCGCGAA |
| | | GGAGGCCTGCCCAACCGGTCTTTACACCCACTCGGG |
| | | GGAGTGTTGTAAAGCATGTAACCTGGGCGAGGGTGT |
| | | GGCGCAGCCCTGCGGGGCCAACCAAACGGTCTGTGA |
| | | GCCTTGCCTGGACAGCGTGACCTTCTCAGATGTGGT |
| | | GTCCGCAACAGAACCATGCAAGCCATGCACTGAGTG |
| | | CGTCGGCCTGCAGTCTATGTCTGCGCCTTGCGTGGA |
| | | GGCTGATGACGCCGTGTGCCGCTGCGCTTACGGCTA |
| | | TTACCAGGATGAGACTACCGGCCGTTGTGAGGCGTG |
| | | CAGGGTTTGTGAGGCTGGCTCCGGTCTGGTATTTTCA |
| | | TGCCAGGACAAACAGAACACGGTGTGCGAGGAGTG |
| | | CCCAGACGGCACTTACTCGGACGAAGCCAATCACGT |
| | | GGACCCCTGCCTGCCGTGCACCGTGTGCGAGGACAC |
| | | TGAGCGGCAGCTTCGCGAATGCACTAGGTGGGCAGA |
| | | TGCTGAGTGCGAGGAGATCCCCGGCCGGTGGATCAC |
| | | CCGCTCCACACCCCCCGAGGGTTCTGATAGCACCGC |
| | | TCCCTCCACCCAGGAGCCCGAAGCCCCCCCGGAACA |
| | | GGACCTGATCGCTAGTACCGTGGCCGGCGTGGTGAC |
| | | TACAGTGATGGGCTCGAGCCAACCTGTGGTCACGCG |
| | | CGGTACCACCGACAACCTCATCCCGGTGTACTGCTC |
| | | AATCCTGGCAGCTGTCGTCGTAGGGCTGGTGGCCTA |
| | | CATCGCGTTCAAGCGTTGGAACTCGTGA |
| 191 | CN1 VL-VH AA | MALPVTALLLPLALLLHAARPSYELTQPPSVSVSPGQT |
| | | ARVTCSGDAQPKAYAYWYQQKSGQAPILVIYGDSKRP |
| | | SGIPERFSGSSSGTMATLTISGAQVEDEADYYCYSTDRS |
| | | GNRVFGGGTRLTVLGGGGSGGGGSGGGGSGGGGSQV |
| | | QLVQSGAEVKKPGASVKVSCKASGYTFSGYAMHWVR |
| | | QAPGQGLEWMGWINPKSPGTNYAQKFQGRVTMTRDT |
| | | SISTAYMELSRLRSDDTAVYYCARYSSSSWGDPWGQG |
| | | TLVTVSSTTTPAPRPPTPAPTIASQPLSLRPEACRPAAGG |
| | | AVHTRGLDFACDIYIWAPLAGTCGVLLLSLVITLYCKR |
| | | GRKKLLYIFKQPFMRPVQTTQEEDGCSCRFPEEEEGGC |
| | | ELRVKFSRSADAPAYKQGQNQLYNELNLGRREEYDVL |
| | | DKRRGRDPEMGGKPRRKNPQEGLYNELQKDKMAEAY |
| | | SEIGMKGERRRGKGHDGLYQGLSTATKDTYDALHMQ |
| | | ALPPRGSGATNFSLLKQAGDVEENPGPMGAGATGRA |
| | | MDGPRLLLLLLLGVSLGGAKEACPTGLYTHSGECCKA |
| | | CNLGEGVAQPCGANQTVCEPCLDSVTFSDVVSATEPC |
| | | KPCTECVGLQSMSAPCVEADDAVCRCAYGYYQDETT |
| | | GRCEACRVCEAGSGLVFSCQDKQNTVCEECPDGTYSD |
| | | EANHVDPCLPCTVCEDTERQLRECTRWADAECEEIPGR |
| | | WITRSTPPEGSDSTAPSTQEPEAPPEQDLIASTVAGVVT |
| | | TVMGSSQPVVTRGTTDNLIPVYCSILAAVVVGLVAYIA |
| | | FKRWNS* |
| 192 | Gap8.3 VH-VL | ATGGCCTTGCCTGTAACCGCCCTGTTGCTGCCCTTGG |
| | | CGCTCCTGCTACACGCCGCCAGGCCCGAAGTGCAGC |
| | | TGCAACAGTCTGGTCCTGAGCTTGTGAAGACCGGGG |
| | | CGTCCGTAAAGATAAGCTGCAAGGCCTCTGGTTATT |
| | | CTTTCACCGGCTACTTCATCCACTGGGTCAAGCAGA |
| | | GCCATGGAAAGTCACTCGAGTGGATTGGATACATCT |
| | | CCTGTTACAATGGCGCAACCAGCTACAACCAGAAGT |
| | | TCAAGGGCAAAGCGACTTTCACCGTGGACACATCCT |
| | | CCTCTACTGCGTACATGCAGTTCAACTCCGTCACCAG |
| | | CGAGGACTCCGCCGTGTACTACTGCGTGCGCAACTA |
| | | TTACGGCAACCTGGACGCTATGGACTATTGGGGCCA |
| | | GGGCACCTCTGTGACGGTGAGCTCTGGCGGCGGGGG |
| | | CTCCGGCGGGGGCGGTAGTGGAGGGGGAGGCTCCG |
| | | GTGGTGGCGGCTCAGACATCGTGATGACCCAGAGCC |
| | | ACAAGTTTATGTCTACCTCCGTGGGCGACCGCGTCTC |
| | | CATCACCTGCAAGGCCTCTCAGGACGTGTCCACCGC |
| | | CGTCGCTTGGTACCAACAGAAGCCCGGACAGTCGCC |
| | | TAAGATCCTGATTTACTCTGCTTCGTACCGCTACACC |
| | | GGCGTGCCCGACAGGTTCACCGGTTCTGGTTCTGGC |
| | | ACCGACTTTACGTTCACCATTAGTTCCGTGCAGGCCG |
| | | AAGATCTGGCGGTGTACTACTGTCAGCAGCATTACA |
| | | GTACTCCGCGCACCTTCGGGGGTGGGACTAAGCTGG |
| | | AGATCAAGACCACGACACCTGCGCCGCGTCCACCCA |
| | | CCCCTGCCCCCACCATCGCCTCCCAGCCCCTGTCCCT |
| | | GAGACCAGAGGCTTGTAGGCCAGCAGCGGGGGGGG |
| | | CAGTGCACACCCGCGGCCTGGATTTCGCTTGTGACA |
| | | TCTACATCTGGGCACCGCTGGCGGGCACTTGCGGGG |
| | | TCCTGCTTCTGTCGCTGGTGATCACCCTGTATTGTAA |
| | | AAGAGGCCGTAAGAAGCTGCTTTATATCTTCAAACA |
| | | GCCCTTCATGCGCCCCGTGCAGACTACCCAGGAGGA |
| | | GGACGGATGCTCTTGTCGCTTCCCTGAGGAAGAGGA |
| | | GGGCGGCTGCGAGCTGCGGGTGAAATTTTCCCGCTC |
| | | GGCGGACGCTCCTGCTTACAAGCAGGGTCAGAACCA |
| | | GCTTTACAACGAGCTCAACCTCGGACGACGTGAGGA |
| | | GTACGACGTGCTGGATAAGCGCCGTGGCCGTGACCC |
| | | GGAGATGGGCGGCAAGCCTCGCCGCAAGAACCCTC |
| | | AGGAAGGACTCTACAACGAGCTGCAGAAGGACAAG |
| | | ATGGCTGAGGCCTACTCCGAGATCGGTATGAAGGGA |
| | | GAGCGGCGCCGGGGTAAAGGCCACGATGGACTGTA |
| | | CCAAGGCCTGTCTACTGCAACCAAAGACACTTACGA |
| | | TGCTCTGCACATGCAGGCCCTGCCCCCTCGGGGCAG |
| | | CGGAGCCACCAACTTTTCGCTGCTGAAGCAGGCCGG |
| | | TGATGTGGAGGAGAACCCCGGGCCAATGGGGGCAG |
| | | GTGCCACAGGCCGCGCCATGGACGGCCCACGCCTCT |
| | | TGCTGCTGCTCTTGCTAGGTGTATCTCTGGGCGGGGC |
| | | CAAGGAGGCGTGCCCAACCGGTCTGTACACCCACTC |
| | | AGGCGAGTGCTGTAAGGCTTGTAATTTAGGCGAGGG |
| | | TGTCGCGCAGCCGTGCGGGGCGAATCAGACGGTTTG |
| | | TGAGCCCTGCCTGGACAGCGTGACATTTAGCGATGT |
| | | GGTATCGGCCACTGAGCCTTGCAAACCGTGCACGGA |
| | | GTGCGTGGGCCTGCAATCCATGTCCGCTCCGTGCGT |
| | | GGAAGCCGATGATGCTGTTTGTCGATGCGCCTACGG |
| | | CTATTACCAGGATGAGACCACCGGACGCTGCGAGGC |
| | | CTGCCGGGTGTGCGAGGCTGGCTCGGGCCTGGTGTT |
| | | CTCATGCCAGGACAAACAGAACACCGTGTGCGAGG |
| | | AGTGTCCCGACGGCACCTACTCCGACGAGGCCAACC |
| | | ATGTTGATCCATGCCTGCCCTGTACCGTCTGTGAAGA |
| | | CACCGAGCGCCAGCTGCGCGAGTGCACTCGCTGGGC |
| | | GGACGCAGAGTGCGAGGAGATCCCGGGCCGCTGGA |
| | | TCACCCGCAGCACTCCGCCTGAAGGCTCCGACAGTA |
| | | CGGCCCCCTCCACCCAGGAGCCAGAGGCTCCCCCGG |
| | | AACAGGACCTGATCGCTTCCACCGTGGCTGGCGTGG |
| | | TCACCACAGTGATGGGCAGCAGCCAGCCCGTGGTCA |
| | | CTCGTGGCACGACGGACAACCTTATCCCCGTCTACT |
| | | GTTCCATTCTCGCGGCCGTGGTGGTTGGATTGGTCGC |
| | | CTATATTGCGTTCAAGCGTTGGAATTCGTGA |
| 193 | Gap8.3 VH-VL AA | MALPVTALLLPLALLLHAARPEVQLQQSGPELVKTGA |
| | | SVKISCKASGYSFTGYFIHWVKQSHGKSLEWIGYISCY |
| | | NGATSYNQKFKGKATFTVDTSSSTAYMQFNSVTSEDS |
| | | AVYYCVRNYYGNLDAMDYWGQGTSVTVSSGGGGSG |
| | | GGGSGGGGSGGGGSDIVMTQSHKFMSTSVGDRVSITC |
| | | KASQDVSTAVAWYQQKPGQSPKILIYSASYRYTGVPD |
| | | RFTGSGSGTDFTFTISSVQAEDLAVYYCQQHYSTPRTF |
| | | GGGTKLEIKTTTPAPRPPTPAPTIASQPLSLRPEACRPAA |
| | | GGAVHTRGLDFACDIYIWAPLAGTCGVLLLSLVITLYC |
| | | KRGRKKLLYIFKQPFMRPVQTTQEEDGCSCRFPEEEEG |
| | | GCELRVKFSRSADAPAYKQGQNQLYNELNLGRREEYD |
| | | VLDKRRGRDPEMGGKPRRKNPQEGLYNELQKDKMAE |
| | | AYSEIGMKGERRRGKGHDGLYQGLSTATKDTYDALH |
| | | MQALPPRGSGATNFSLLKQAGDVEENPGPMGAGATG |
| | | RAMDGPRLLLLLLLGVSLGGAKEACPTGLYTHSGECC |
| | | KACNLGEGVAQPCGANQTVCEPCLDSVTFSDVVSATE |
| | | PCKPCTECVGLQSMSAPCVEADDAVCRCAYGYYQDE |
| | | TTGRCEACRVCEAGSGLVFSCQDKQNTVCEECPDGTY |
| | | SDEANHVDPCLPCTVCEDTERQLRECTRWADAECEEIP |
| | | GRWITRSTPPEGSDSTAPSTQEPEAPPEQDLIASTVAGV |
| | | VTTVMGSSQPVVTRGTTDNLIPVYCSILAAVVVGLVA |
| | | YIAFKRWNS |
| 194 | Gap8.3 VL-VH | ATGGCCCTGCCTGTGACTGCATTACTGCTGCCACTGG |
| | | CCCTGCTGTTGCACGCCGCCAGGCCCGACATCGTGA |
| | | TGACCCAGAGCCACAAGTTTATGTCTACTAGCGTGG |
| | | GGGACAGGGTGTCTATCACCTGCAAGGCTTCCCAGG |
| | | ATGTCAGCACTGCTGTCGCGTGGTACCAACAGAAGC |
| | | CCGGCCAGTCTCCGAAGATTCTGATCTATTCGGCGA |
| | | GCTACCGCTACACCGGCGTGCCCGACCGCTTCACCG |
| | | GCTCCGGTTCAGGAACCGACTTTACTTTCACAATTTC |
| | | TTCGGTGCAGGCCGAGGACCTGGCGGTTTACTACTG |
| | | CCAGCAGCACTACTCCACCCCGCGCACCTTCGGTGG |
| | | CGGCACCAAGCTGGAGATCAAGGGAGGCGGAGGCT |
| | | CCGGAGGCGGCGGCTCTGGTGGTGGCGGCTCTGGCG |
| | | GGGGCGGTTCCGAGGTGCAGCTGCAGCAATCCGGCC |
| | | CTGAGCTGGTGAAGACCGGCGCCTCCGTGAAGATCT |
| | | CTTGCAAGGCCTCTGGATACTCCTTCACCGGCTATTT |
| | | CATCCACTGGGTCAAGCAGAGTCATGGAAAGTCGCT |
| | | GGAGTGGATTGGGTATATCTCATGCTACAACGGCGC |
| | | TACCTCCTATAACCAGAAATTCAAAGGTAAGGCAAC |
| | | CTTTACCGTGGACACTAGCTCGTCCACCGCCTACATG |
| | | CAGTTCAACTCGGTCACATCCGAAGACTCCGCCGTG |
| | | TATTACTGCGTGCGCAACTACTACGGCAACCTGGAT |
| | | GCCATGGATTATTGGGGCCAGGGTACCTCGGTAACC |
| | | GTGTCCTCCACAACAACCCCCGCCCCGCGTCCCCCT |
| | | ACCCCCGCTCCAACCATCGCTTCTCAGCCGCTGAGTC |
| | | TTCGGCCCGAGGCGTGCCGACCGGCTGCTGGCGGCG |
| | | CGGTCCACACGCGTGGCCTCGACTTCGCCTGTGACA |
| | | TCTACATATGGGCGCCCCTGGCCGGCACCTGCGGGG |
| | | TGCTGCTGTTGAGCCTGGTGATTACCCTGTACTGTAA |
| | | ACGGGGCCGCAAGAAGCTCCTCTACATCTTCAAGCA |
| | | GCCCTTCATGAGACCTGTGCAGACGACCCAGGAGGA |
| | | GGATGGGTGCTCTTGTCGTTTCCCCGAAGAGGAGGA |
| | | AGGGGGGTGCGAGCTGCGCGTGAAATTTTCGCGCAG |
| | | CGCCGACGCGCCTGCCTACAAGCAGGGCCAGAACCA |
| | | ACTTTACAACGAGCTCAACCTGGGCAGAAGGGAGG |
| | | AGTACGACGTGCTTGACAAGCGCCGTGGTCGGGATC |
| | | CAGAGATGGGAGGCAAACCGCGGCGCAAGAACCCT |
| | | CAGGAGGGCCTCTACAACGAGCTTCAGAAGGACAA |
| | | AATGGCTGAGGCGTACAGCGAGATCGGTATGAAGG |
| | | GCGAGCGGCGCCGCGGGAAGGGACATGACGGTCTC |
| | | TACCAGGGTCTGTCCACGGCCACCAAGGATACCTAC |
| | | GATGCTTTGCACATGCAGGCCCTGCCCCCGCGCGGC |
| | | AGCGGGGCCACCAACTTTTCCCTACTGAAGCAGGCC |
| | | GGGGATGTGGAGGAGAATCCAGGGCCCATGGGCGC |
| | | GGGCGCCACTGGACGCGCCATGGACGGCCCCCGCCT |
| | | GCTGCTGCTACTACTTTTAGGGGTGTCCTTGGGTGGC |
| | | GCCAAGGAGGCGTGCCCAACCGGACTGTACACGCAT |
| | | AGCGGTGAGTGCTGTAAGGCCTGTAACCTCGGGGAG |
| | | GGCGTCGCGCAGCCATGTGGCGCTAACCAGACTGTC |
| | | TGCGAGCCATGCTTGGATTCTGTCACCTTTAGCGACG |
| | | TGGTGTCTGCAACTGAGCCATGTAAACCCTGCACGG |
| | | AGTGCGTGGGACTGCAGTCTATGTCCGCCCCTTGCG |
| | | TGGAGGCAGATGACGCAGTTTGTCGATGTGCCTATG |
| | | GCTACTATCAAGACGAGACCACCGGCAGGTGTGAAG |
| | | CGTGCCGCGTGTGCGAGGCTGGGTCCGGATTGGTGT |
| | | TCTCATGCCAGGACAAACAGAACACGGTGTGCGAGG |
| | | AGTGTCCTGACGGCACCTACAGTGACGAGGCTAATC |
| | | ACGTGGACCCGTGCCTGCCCTGTACGGTCTGTGAAG |
| | | ACACCGAGCGCCAGCTGCGCGAATGCACACGTTGGG |
| | | CGGACGCCGAGTGCGAGGAGATTCCCGGTCGCTGGA |
| | | TCACCCGCAGTACTCCTCCTGAAGGGAGTGACAGTA |
| | | CAGCCCCTTCCACCCAGGAGCCCGAGGCTCCCCCCG |
| | | AACAGGATCTGATCGCATCTACTGTCGCTGGTGTAG |
| | | TGACGACCGTGATGGGTAGCTCGCAGCCGGTCGTGA |
| | | CTCGTGGGACCACAGACAACCTCATCCCGGTGTACT |
| | | GTTCCATCCTGGCTGCTGTGGTTGTGGGCCTGGTAGC |
| | | CTACATCGCGTTCAAGCGTTGGAATTCCTGA |
| 195 | Gap8.3 VL-VH AA | MALPVTALLLPLALLLHAARPDIVMTQSHKFMSTSVG |
| | | DRVSITCKASQDVSTAVAWYQQKPGQSPKILIYSASYR |
| | | YTGVPDRFTGSGSGTDFTFTISSVQAEDLAVYYCQQHY |
| | | STPRTFGGGTKLEIKGGGGSGGGGSGGGGSGGGGSEV |
| | | QLQQSGPELVKTGASVKISCKASGYSFTGYFIHWVKQS |
| | | HGKSLEWIGYISCYNGATSYNQKFKGKATFTVDTSSST |
| | | AYMQFNSVTSEDSAVYYCVRNYYGNLDAMDYWGQG |
| | | TSVTVSSTTTPAPRPPTPAPTIASQPLSLRPEACRPAAGG |
| | | AVHTRGLDFACDIYIWAPLAGTCGVLLLSLVITLYCKR |
| | | GRKKLLYIFKQPFMRPVQTTQEEDGCSCRFPEEEEGGC |
| | | ELRVKFSRSADAPAYKQGQNQLYNELNLGRREEYDVL |
| | | DKRRGRDPEMGGKPRRKNPQEGLYNELQKDKMAEAY |
| | | SEIGMKGERRRGKGHDGLYQGLSTATKDTYDALHMQ |
| | | ALPPRGSGATNFSLLKQAGDVEENPGPMGAGATGRA |
| | | MDGPRLLLLLLLGVSLGGAKEACPTGLYTHSGECCKA |
| | | CNLGEGVAQPCGANQTVCEPCLDSVTFSDVVSATEPC |
| | | KPCTECVGLQSMSAPCVEADDAVCRCAYGYYQDETT |
| | | GRCEACRVCEAGSGLVFSCQDKQNTVCEECPDGTYSD |
| | | EANHVDPCLPCTVCEDTERQLRECTRWADAECEEIPGR |
| | | WITRSTPPEGSDSTAPSTQEPEAPPEQDLIASTVAGVVT |
| | | TVMGSSQPVVTRGTTDNLIPVYCSILAAVVVGLVAYIA |
| | | FKRWNS* |
| 196 | mAb104 VH-VL | ATGGCTCTGCCGGTGACTGCTCTGCTGCTGCCTTTGG |
| | | CGCTGCTGCTTCATGCCGCTAGGCCCGAGGTGCAGC |
| | | TGGTGGAGAGCGGTGGCGATCTCGTGCAGCCGGGGG |
| | | GGTCGCTCAAGCTTTCGTGTACAGCATCTGGCTTCAC |
| | | TTTTTCCAACTACGGCATGTCCTGGATTCGGCAGACG |
| | | CCCGACAAGCGGCTGGAGTGGGTGGCAACCATCGTG |
| | | GGCAACGACTACACCTACTTCCCGGACTCCATGAAA |
| | | GGCCGTTTCACCGTGTCTCGAGATAACGCGAAGTCT |
| | | ATCCTGTACCTGCAGATGAACAGCCTGGCCTCCGCC |
| | | GACACGGCGATGTATTACTGCACTCGTCATGACTGG |
| | | GTCTTCGACTATTGGGGCCAGGGTACTCCGCTGACC |
| | | GTGAGCTCCGGTGGAGGTGGCTCTGGAGGCGGAGG |
| | | GTCCGGAGGTGGCGGGTCCGGCGGCGGCGGCAGTG |
| | | ACATTGTCCTCACCCAGAGCCCCGCTTCTCTCGCCGT |
| | | GTCCCTGGGTCAACGCGCCATCCTGTCATGCAAGGC |
| | | ATCACAGAGTGTGAGCTTCGCCGGCTCCTCGCTGAT |
| | | GCACTGGTACCAACAGAAGCCTGGACAGCAGCCCA |
| | | AGCTCCTGATTTACCGCGCCTCCGACCTGGAGACTG |
| | | GCATCCCAACCAGGTTCAGTGGTGGCGGTTCTGGCA |
| | | CCGACTTTACCCTAAACATCCACCCCGTAGAGGAAG |
| | | ATGACGCAGCCACCTATTACTGCCAACAGTCCAGGG |
| | | AGTACCCCTATACCTTCGGCGGCGGCACCCGCTTAG |
| | | AAATTAAGACCACAACCCCCGCGCCCCGGCCCCCAA |
| | | CCCCAGCGCCGACCATTGCTAGCCAGCCACTGTCCC |
| | | TGCGCCCTGAGGCGTGCCGACCAGCAGCTGGAGGCG |
| | | CCGTCCACACTCGCGGTTTGGACTTCGCGTGTGACAT |
| | | CTACATATGGGCCCCCCTGGCCGGCACCTGCGGGGT |
| | | GCTGCTGTTGTCTCTGGTCATCACCCTGTACTGTAAG |
| | | CGCGGCCGCAAGAAGCTGTTATACATCTTCAAGCAA |
| | | CCCTTCATGCGTCCGGTGCAGACAACCCAGGAAGAG |
| | | GACGGGTGCTCTTGCAGATTTCCTGAAGAGGAGGAG |
| | | GGAGGGTGCGAGCTACGCGTGAAATTTAGCCGCTCC |
| | | GCCGACGCGCCGGCCTACAAGCAGGGTCAGAACCA |
| | | GCTGTACAACGAGCTGAACCTGGGCCGCCGCGAGGA |
| | | GTACGACGTGTTGGATAAGCGCCGTGGACGCGATCC |
| | | GGAGATGGGGGGCAAACCGCGCCGTAAAAACCCTC |
| | | AGGAGGGCCTCTACAACGAGCTTCAGAAGGACAAG |
| | | ATGGCCGAGGCCTACTCCGAGATCGGTATGAAGGGC |
| | | GAGCGGCGCCGTGGCAAAGGCCACGACGGCCTCTAC |
| | | CAGGGCCTGAGCACGGCAACCAAGGATACCTACGAT |
| | | GCTCTGCACATGCAGGCCCTGCCCCCGCGTGGCTCC |
| | | GGTGCGACCAACTTTTCTCTGCTGAAGCAGGCTGGT |
| | | GATGTGGAAGAGAATCCTGGCCCTATGGGCGCTGGG |
| | | GCTACCGGAAGAGCCATGGATGGCCCCCGCTTGCTG |
| | | CTTCTCCTGCTCTTGGGTGTCAGTCTGGGCGGAGCCA |
| | | AGGAGGCGTGCCCAACGGGGCTTTATACACACTCCG |
| | | GGGAATGTTGTAAGGCCTGCAATCTGGGTGAGGGCG |
| | | TGGCCCAGCCATGCGGGGCCAACCAGACTGTCTGTG |
| | | AACCATGTCTGGATTCCGTGACCTTCAGCGACGTGG |
| | | TGTCGGCTACGGAGCCCTGCAAGCCCTGCACCGAGT |
| | | GCGTGGGACTTCAGTCGATGTCTGCTCCCTGCGTGG |
| | | AGGCCGATGACGCCGTGTGCCGCTGCGCCTACGGCT |
| | | ACTACCAGGACGAGACTACCGGCCGCTGTGAAGCCT |
| | | GTCGCGTCTGTGAGGCTGGCTCCGGTTTGGTGTTCTC |
| | | ATGCCAGGATAAACAGAACACGGTTTGCGAGGAGT |
| | | GTCCCGACGGAACTTACTCAGACGAGGCCAATCACG |
| | | TTGACCCGTGCCTGCCGTGCACCGTCTGTGAGGACA |
| | | CCGAGCGCCAGCTGAGGGAGTGCACCCGCTGGGCG |
| | | GACGCTGAGTGCGAGGAGATCCCCGGGCGCTGGATC |
| | | ACTCGCTCGACCCCTCCCGAGGGCTCTGACAGCACT |
| | | GCTCCCAGTACCCAGGAGCCCGAGGCTCCTCCTGAA |
| | | CAGGACCTTATCGCCTCCACAGTAGCCGGCGTGGTC |
| | | ACCACCGTGATGGGCTCGTCCCAGCCTGTGGTGACC |
| | | CGCGGCACGACTGACAACCTGATCCCTGTATACTGT |
| | | AGCATCCTGGCGGCCGTGGTGGTAGGGCTAGTGGCG |
| | | TATATCGCGTTCAAACGATGGAATTCCTGA |
| 197 | mAb104 VH-VL | MALPVTALLLPLALLLHAARPEVQLVESGGDLVQPGG |
| | AA | SLKLSCTASGFTFSNYGMSWIRQTPDKRLEWVATIVGN |
| | | DYTYFPDSMKGRFTVSRDNAKSILYLQMNSLASADTA |
| | | MYYCTRHDWVFDYWGQGTPLTVSSGGGGSGGGGSG |
| | | GGGSGGGGSDIVLTQSPASLAVSLGQRAILSCKASQSV |
| | | SFAGSSLMHWYQQKPGQQPKLLIYRASDLETGIPTRES |
| | | GGGSGTDFTLNIHPVEEDDAATYYCQQSREYPYTFGG |
| | | GTRLEIKTTTPAPRPPTPAPTIASQPLSLRPEACRPAAGG |
| | | AVHTRGLDFACDIYIWAPLAGTCGVLLLSLVITLYCKR |
| | | GRKKLLYIFKQPFMRPVQTTQEEDGCSCRFPEEEEGGC |
| | | ELRVKFSRSADAPAYKQGQNQLYNELNLGRREEYDVL |
| | | DKRRGRDPEMGGKPRRKNPQEGLYNELQKDKMAEAY |
| | | SEIGMKGERRRGKGHDGLYQGLSTATKDTYDALHMQ |
| | | ALPPRGSGATNFSLLKQAGDVEENPGPMGAGATGRA |
| | | MDGPRLLLLLLLGVSLGGAKEACPTGLYTHSGECCKA |
| | | CNLGEGVAQPCGANQTVCEPCLDSVTFSDVVSATEPC |
| | | KPCTECVGLQSMSAPCVEADDAVCRCAYGYYQDETT |
| | | GRCEACRVCEAGSGLVFSCQDKQNTVCEECPDGTYSD |
| | | EANHVDPCLPCTVCEDTERQLRECTRWADAECEEIPGR |
| | | WITRSTPPEGSDSTAPSTQEPEAPPEQDLIASTVAGVVT |
| | | TVMGSSQPVVTRGTTDNLIPVYCSILAAVVVGLVAYIA |
| | | FKRWNS* |
| 198 | mAb104 VL-VH | ATGGCGCTGCCTGTCACGGCCTTGCTACTCCCCCTGG |
| | | CTCTGCTTCTGCACGCCGCCCGTCCCGACATCGTACT |
| | | GACCCAGAGCCCAGCGAGCCTTGCGGTGAGCCTGGG |
| | | GCAGCGCGCCATCCTGTCTTGTAAGGCTTCTCAGTCC |
| | | GTGTCCTTCGCGGGTTCCAGCCTGATGCACTGGTACC |
| | | AGCAGAAGCCTGGTCAGCAGCCCAAATTGCTGATCT |
| | | ACCGCGCTTCCGACCTGGAGACTGGCATCCCTACCC |
| | | GTTTTTCCGGAGGAGGCTCCGGCACCGACTTCACGC |
| | | TCAACATCCACCCCGTTGAGGAAGATGACGCGGCTA |
| | | CTTACTACTGTCAACAGAGCCGCGAGTACCCCTATA |
| | | CCTTCGGCGGAGGAACGAGGTTGGAGATCAAGGGC |
| | | GGCGGAGGGTCAGGAGGCGGAGGCTCTGGCGGCGG |
| | | TGGAAGCGGGGGTGGCGGCTCCGAGGTGCAGCTGGT |
| | | AGAGAGCGGCGGGGACCTGGTGCAGCCTGGTGGCTC |
| | | CTTAAAGCTTTCTTGCACCGCTTCCGGCTTCACCTTT |
| | | AGCAACTACGGTATGAGTTGGATTCGTCAGACCCCG |
| | | GATAAGCGCCTGGAGTGGGTCGCAACAATTGTCGGC |
| | | AACGACTACACCTACTTCCCAGATTCTATGAAGGGG |
| | | CGCTTCACCGTGTCGCGCGACAACGCGAAGAGCATC |
| | | CTGTACCTGCAGATGAACTCCTTGGCCTCTGCTGATA |
| | | CCGCCATGTATTACTGTACCCGCCATGACTGGGTGTT |
| | | CGACTATTGGGGCCAGGGCACCCCTTTGACAGTGTC |
| | | GTCCACTACAACCCCCGCTCCTAGGCCACCAACCCC |
| | | AGCCCCAACCATCGCCTCCCAGCCACTCTCGCTGCG |
| | | CCCCGAGGCTTGCCGGCCGGCGGCCGGGGGGGCTGT |
| | | CCACACCCGTGGATTGGACTTCGCATGCGACATTTA |
| | | TATTTGGGCACCCCTGGCTGGGACCTGCGGTGTACT |
| | | GCTGTTGTCCCTGGTGATAACCCTGTACTGTAAGCGC |
| | | GGCCGAAAGAAGCTGCTGTATATCTTCAAACAGCCC |
| | | TTCATGCGTCCTGTGCAGACAACCCAGGAGGAAGAC |
| | | GGGTGCTCGTGCCGGTTTCCTGAGGAGGAGGAGGGT |
| | | GGGTGCGAGCTGCGCGTGAAATTTTCCCGTTCCGCT |
| | | GACGCCCCAGCCTACAAGCAAGGACAGAACCAGCT |
| | | CTACAACGAGCTTAACCTGGGCCGCAGAGAGGAGTA |
| | | CGACGTGCTCGACAAACGCCGCGGTCGTGACCCGGA |
| | | GATGGGCGGCAAGCCCCGCCGGAAGAATCCTCAGG |
| | | AGGGCCTATACAACGAGTTGCAGAAGGACAAGATG |
| | | GCTGAAGCATACTCGGAGATTGGAATGAAAGGCGA |
| | | GCGGCGCCGGGGCAAGGGCCACGACGGCTTATATCA |
| | | GGGTCTTTCGACGGCAACGAAAGACACCTACGATGC |
| | | TCTTCATATGCAGGCCCTGCCGCCGCGCGGTTCCGG |
| | | AGCTACCAACTTTTCATTGCTGAAGCAGGCCGGAGA |
| | | TGTGGAGGAGAATCCCGGCCCTATGGGGGCCGGGGC |
| | | CACCGGTAGAGCCATGGATGGCCCCCGACTGCTGCT |
| | | CCTCCTTTTACTAGGCGTTTCTCTGGGCGGCGCCAAG |
| | | GAGGCCTGCCCAACCGGTCTGTACACCCACTCCGGT |
| | | GAGTGTTGTAAGGCCTGCAACCTGGGTGAGGGCGTG |
| | | GCGCAGCCCTGCGGGGCCAACCAGACTGTCTGTGAA |
| | | CCGTGCCTGGATTCTGTGACTTTCAGCGATGTGGTTA |
| | | GTGCAACCGAGCCTTGCAAACCCTGTACTGAGTGCG |
| | | TGGGCCTGCAGTCCATGTCTGCTCCGTGCGTGGAAG |
| | | CGGATGACGCCGTCTGTAGGTGTGCCTACGGCTACT |
| | | ACCAAGACGAGACCACAGGCAGGTGTGAGGCTTGTC |
| | | GAGTGTGCGAGGCTGGTTCGGGCCTTGTCTTCTCATG |
| | | CCAGGACAAGCAGAATACGGTCTGTGAGGAGTGTCC |
| | | CGACGGCACTTACTCCGACGAGGCCAACCACGTGGA |
| | | TCCTTGCCTGCCCTGCACTGTTTGCGAAGACACCGA |
| | | GCGCCAGCTGCGCGAGTGTACCCGCTGGGCAGACGC |
| | | CGAGTGCGAGGAGATCCCCGGCCGCTGGATCACTCG |
| | | CTCGACTCCGCCTGAAGGCAGTGACAGCACTGCTCC |
| | | CTCTACGCAGGAGCCCGAGGCTCCCCCGGAACAGGA |
| | | CCTCATCGCCTCTACCGTGGCGGGCGTCGTTACCACC |
| | | GTGATGGGCTCCAGCCAGCCGGTGGTCACCAGAGGC |
| | | ACTACAGACAACCTGATCCCGGTGTACTGCTCTATC |
| | | CTGGCGGCGGTGGTGGTGGGCCTCGTGGCCTACATC |
| | | GCGTTCAAGCGGTGGAATTCGTGA |
| 199 | mAb104 VL-VH | MALPVTALLLPLALLLHAARPDIVLTQSPASLAVSLGQ |
| | AA | RAILSCKASQSVSFAGSSLMHWYQQKPGQQPKLLIYRA |
| | | SDLETGIPTRESGGGSGTDFTLNIHPVEEDDAATYYCQ |
| | | QSREYPYTFGGGTRLEIKGGGGSGGGGSGGGGSGGGG |
| | | SEVQLVESGGDLVQPGGSLKLSCTASGFTFSNYGMSWI |
| | | RQTPDKRLEWVATIVGNDYTYFPDSMKGRFTVSRDNA |
| | | KSILYLQMNSLASADTAMYYCTRHDWVFDYWGQGTP |
| | | LTVSSTTTPAPRPPTPAPTIASQPLSLRPEACRPAAGGA |
| | | VHTRGLDFACDIYIWAPLAGTCGVLLLSLVITLYCKRG |
| | | RKKLLYIFKQPFMRPVQTTQEEDGCSCRFPEEEEGGCE |
| | | LRVKFSRSADAPAYKQGQNQLYNELNLGRREEYDVLD |
| | | KRRGRDPEMGGKPRRKNPQEGLYNELQKDKMAEAYS |
| | | EIGMKGERRRGKGHDGLYQGLSTATKDTYDALHMQA |
| | | LPPRGSGATNFSLLKQAGDVEENPGPMGAGATGRAM |
| | | DGPRLLLLLLLGVSLGGAKEACPTGLYTHSGECCKAC |
| | | NLGEGVAQPCGANQTVCEPCLDSVTFSDVVSATEPCK |
| | | PCTECVGLQSMSAPCVEADDAVCRCAYGYYQDETTG |
| | | RCEACRVCEAGSGLVFSCQDKQNTVCEECPDGTYSDE |
| | | ANHVDPCLPCTVCEDTERQLRECTRWADAECEEIPGR |
| | | WITRSTPPEGSDSTAPSTQEPEAPPEQDLIASTVAGVVT |
| | | TVMGSSQPVVTRGTTDNLIPVYCSILAAVVVGLVAYIA |
| | | FKRWNS* |
| 200 | NOV45 VH-VL | ATGGCGTTGCCAGTGACTGCTCTGCTGCTGCCCCTAG |
| | | CTCTCCTCCTGCACGCCGCGAGGCCTCAGGTCCAGC |
| | | TGGTGGAGAGCGGAGCGGAGGTGAAAAAGCCAGGC |
| | | GCCTCCGTGAAGGTGTCCTGTAAGGCCTCCGGCTAC |
| | | ACCTTCACCAACTACATCATCCACTGGGTCAAGCAG |
| | | GAGCCCGGCCAGGGCCTGGAGTGGATTGGCTATTTC |
| | | AACCCCTATAACCATGGAACCAAGTACAACGAGAA |
| | | ATTTAAGGGCAGAGCCACTCTGACAGCCGATAAGAG |
| | | CATCTCTACCGCGTACATGGAGCTGTCCTCCCTACGG |
| | | AGCGAGGATACGGCCGTATACTACTGCGCTCGCTCT |
| | | GGACCCTATGCGTGGTTTGACACCTGGGGCCAGGGC |
| | | ACGACCGTGACTGTATCGTCCGGGGGCGGCGGGAGT |
| | | GGCGGCGGTGGCTCCGGCGGTGGCGGCAGCGGCGG |
| | | AGGGGGTTCCGACATCCTTCTGACCCAGAGCCCTGC |
| | | AACCCTGAGCCTGTCGCCTGGGGAGCGGGCAACTTT |
| | | GTCCTGTCGAGCCTCTCAGAACATCGGTACTTCCATC |
| | | CAGTGGTACCAGCAGAAGCCTGGACAGGCCCCGCGC |
| | | CTCCTCATCCGCAGCTCCTCCGAGAGCATTTCTGGTA |
| | | TCTCTTCCCGCTTCAGCGGATCGGGATCTGGGACGG |
| | | ACTTCACGTTGACAATCAGTTCGCTGGAACCTGAGG |
| | | ACTTTGCAGTGTACTACTGTCAACAGTCAAATACCT |
| | | GGCCGTTCACCTTTGGCCAGGGCACCAAGCTGGAGA |
| | | TCAAGACCACTACCCCTGCCCCCCGCCCGCCTACCC |
| | | CCGCGCCGACCATCGCCTCTCAGCCCCTGTCTCTGCG |
| | | CCCTGAAGCTTGTCGCCCCGCTGCAGGCGGAGCCGT |
| | | GCACACACGGGGTCTTGATTTCGCATGTGACATCTA |
| | | TATATGGGCCCCCCTGGCTGGCACCTGCGGGGTGCT |
| | | GCTATTGTCCCTAGTCATCACCCTTTATTGCAAGCGC |
| | | GGCCGCAAGAAGCTCCTGTACATCTTCAAACAGCCC |
| | | TTCATGCGCCCCGTGCAGACGACCCAGGAGGAGGAC |
| | | GGGTGCAGCTGTAGGTTCCCCGAAGAAGAGGAGGG |
| | | TGGGTGCGAGCTTCGCGTTAAATTCTCCCGTAGCGC |
| | | GGACGCTCCTGCCTACAAGCAAGGTCAGAACCAGTT |
| | | GTACAACGAGCTCAACCTGGGCCGCCGCGAGGAGTA |
| | | CGATGTCCTCGACAAACGTCGTGGTAGAGACCCGGA |
| | | GATGGGGGGCAAGCCGCGTCGAAAGAACCCTCAGG |
| | | AGGGCCTTTACAACGAGCTCCAGAAGGACAAGATG |
| | | GCTGAGGCTTACTCTGAGATCGGAATGAAAGGCGAG |
| | | CGGCGTCGCGGTAAGGGCCATGACGGCCTGTACCAA |
| | | GGCCTTAGTACTGCCACCAAAGACACCTACGACGCG |
| | | CTGCACATGCAGGCCCTGCCTCCGCGTGGTTCTGGC |
| | | GCCACGAACTTTTCATTACTGAAGCAGGCTGGGGAC |
| | | GTGGAGGAGAATCCAGGCCCTATGGGCGCGGGTGCC |
| | | ACTGGTCGCGCCATGGATGGCCCCCGCCTGCTTCTG |
| | | CTGTTACTGCTTGGCGTGTCCTTAGGGGGCGCTAAG |
| | | GAGGCGTGCCCAACCGGTCTGTACACCCACTCCGGA |
| | | GAATGCTGTAAAGCCTGCAATCTGGGCGAGGGCGTG |
| | | GCGCAGCCATGCGGGGCGAACCAGACCGTATGCGA |
| | | GCCATGCCTGGATAGTGTGACCTTCTCAGACGTGGT |
| | | GTCCGCCACCGAGCCGTGCAAGCCGTGCACTGAGTG |
| | | CGTGGGTCTGCAGTCTATGTCTGCTCCCTGCGTGGAG |
| | | GCAGATGACGCTGTCTGTAGGTGTGCCTACGGCTAC |
| | | TACCAAGACGAGACCACCGGCAGATGTGAGGCTTGT |
| | | CGCGTGTGCGAGGCTGGGTCGGGCTTGGTGTTCTCA |
| | | TGCCAGGATAAGCAGAACACGGTCTGCGAGGAGTGT |
| | | CCGGACGGCACTTACTCCGACGAGGCCAACCACGTG |
| | | GATCCATGCCTGCCCTGCACTGTCTGTGAAGACACC |
| | | GAGCGCCAGCTCCGGGAGTGCACACGCTGGGCAGA |
| | | CGCCGAGTGCGAGGAGATCCCCGGCCGCTGGATAAC |
| | | CCGCTCGACTCCGCCTGAAGGTAGCGATTCCACAGC |
| | | TCCCTCCACCCAGGAGCCCGAGGCCCCCCCCGAACA |
| | | GGACCTGATTGCTTCCACCGTGGCGGGCGTTGTGAC |
| | | CACCGTGATGGGCAGTAGCCAGCCGGTGGTCACCCG |
| | | CGGAACAACCGACAACCTGATTCCGGTTTACTGTTC |
| | | TATCCTGGCCGCCGTTGTGGTGGGCCTGGTCGCGTAT |
| | | ATTGCGTTCAAGCGTTGGAATTCCTGA |
| 201 | NOV45 VH-VL AA | MALPVTALLLPLALLLHAARPQVQLVESGAEVKKPGA |
| | | SVKVSCKASGYTFTNYIIHWVKQEPGQGLEWIGYFNPY |
| | | NHGTKYNEKFKGRATLTADKSISTAYMELSSLRSEDTA |
| | | VYYCARSGPYAWFDTWGQGTTVTVSSGGGGSGGGGS |
| | | GGGGSGGGGSDILLTQSPATLSLSPGERATLSCRASQNI |
| | | GTSIQWYQQKPGQAPRLLIRSSSESISGISSRFSGSGSGT |
| | | DFTLTISSLEPEDFAVYYCQQSNTWPFTFGQGTKLEIKT |
| | | TTPAPRPPTPAPTIASQPLSLRPEACRPAAGGAVHTRGL |
| | | DFACDIYIWAPLAGTCGVLLLSLVITLYCKRGRKKLLYI |
| | | FKQPFMRPVQTTQEEDGCSCRFPEEEEGGCELRVKFSR |
| | | SADAPAYKQGQNQLYNELNLGRREEYDVLDKRRGRD |
| | | PEMGGKPRRKNPQEGLYNELQKDKMAEAYSEIGMKG |
| | | ERRRGKGHDGLYQGLSTATKDTYDALHMQALPPRGS |
| | | GATNFSLLKQAGDVEENPGPMGAGATGRAMDGPRLL |
| | | LLLLLGVSLGGAKEACPTGLYTHSGECCKACNLGEGV |
| | | AQPCGANQTVCEPCLDSVTFSDVVSATEPCKPCTECVG |
| | | LQSMSAPCVEADDAVCRCAYGYYQDETTGRCEACRV |
| | | CEAGSGLVFSCQDKQNTVCEECPDGTYSDEANHVDPC |
| | | LPCTVCEDTERQLRECTRWADAECEEIPGRWITRSTPPE |
| | | GSDSTAPSTQEPEAPPEQDLIASTVAGVVTTVMGSSQP |
| | | VVTRGTTDNLIPVYCSILAAVVVGLVAYIAFKRWNS* |
| 202 | NOV45 VL-VH | ATGGCCCTGCCAGTAACTGCACTTCTGCTACCTTTGG |
| | | CTCTGCTCCTGCACGCCGCTCGCCCTGACATCTTGCT |
| | | CACCCAGAGCCCTGCAACTCTAAGCCTCTCACCGGG |
| | | CGAAAGGGCCACTCTTTCCTGTCGGGCGTCACAGAA |
| | | CATCGGCACCAGCATCCAGTGGTACCAGCAGAAGCC |
| | | CGGCCAGGCCCCCAGGCTGCTCATTCGCAGTAGTTC |
| | | CGAAAGCATCTCTGGCATCTCGTCCCGCTTCTCCGGC |
| | | AGCGGGTCGGGTACCGATTTCACCCTGACCATCTCA |
| | | TCGCTCGAGCCAGAAGACTTTGCTGTCTACTACTGCC |
| | | AACAGTCAAATACCTGGCCGTTCACCTTCGGCCAGG |
| | | GCACCAAGCTGGAGATTAAAGGTGGCGGTGGCTCCG |
| | | GTGGAGGCGGCTCGGGAGGCGGCGGCTCCGGGGGT |
| | | GGCGGATCCCAGGTCCAACTGGTGGAGAGCGGCGC |
| | | GGAGGTGAAGAAGCCTGGGGCTTCCGTGAAGGTGTC |
| | | TTGTAAGGCTTCCGGCTACACGTTCACGAACTACAT |
| | | CATCCACTGGGTGAAGCAGGAGCCCGGGCAGGGAC |
| | | TCGAGTGGATTGGCTATTTCAACCCCTATAATCACG |
| | | GCACCAAGTACAACGAGAAGTTTAAGGGGCGCGCT |
| | | ACACTCACTGCGGACAAGAGCATCAGCACCGCTTAC |
| | | ATGGAGTTGTCCAGTCTGCGCTCGGAAGATACAGCC |
| | | GTGTACTATTGCGCTCGCTCTGGGCCGTACGCGTGGT |
| | | TCGACACCTGGGGCCAGGGCACGACCGTCACCGTGT |
| | | CCTCTACCACGACCCCGGCTCCGCGCCCTCCCACTCC |
| | | CGCTCCTACCATTGCTTCCCAACCCCTGAGTTTGAGA |
| | | CCTGAGGCTTGCCGTCCTGCAGCAGGCGGAGCCGTC |
| | | CACACACGTGGCCTGGACTTCGCCTGTGACATCTAC |
| | | ATCTGGGCGCCCCTGGCCGGAACCTGCGGGGTACTG |
| | | CTCCTGAGCCTGGTGATTACGCTGTACTGTAAACGC |
| | | GGTCGAAAGAAACTGCTTTATATCTTTAAACAGCCC |
| | | TTCATGCGTCCCGTGCAGACGACCCAGGAGGAGGAT |
| | | GGCTGTTCCTGCCGTTTCCCCGAGGAAGAGGAGGGC |
| | | GGTTGCGAGCTGAGAGTTAAATTTTCGCGCAGCGCG |
| | | GACGCCCCCGCCTACAAGCAGGGTCAGAATCAGCTG |
| | | TACAACGAGCTCAACCTGGGTCGCCGCGAGGAGTAC |
| | | GACGTGTTGGATAAAAGGCGCGGCCGCGACCCGGA |
| | | GATGGGCGGCAAGCCGAGACGCAAGAACCCTCAGG |
| | | AGGGCCTTTACAACGAGCTGCAGAAGGACAAGATG |
| | | GCTGAGGCCTACTCCGAGATAGGTATGAAGGGCGAG |
| | | CGTCGCCGTGGAAAGGGTCATGACGGCCTATATCAG |
| | | GGCCTGTCCACCGCCACGAAGGACACTTACGACGCG |
| | | CTGCACATGCAGGCTCTTCCTCCCCGCGGTAGTGGG |
| | | GCCACCAATTTTTCCTTGCTGAAGCAGGCCGGTGAT |
| | | GTGGAGGAGAACCCAGGGCCCATGGGTGCTGGCGC |
| | | CACCGGCCGGGCCATGGATGGGCCGCGCCTGCTGCT |
| | | GCTGCTTCTGCTGGGCGTGTCGCTGGGCGGCGCCAA |
| | | GGAGGCGTGCCCAACCGGTCTTTACACCCATTCTGG |
| | | CGAGTGTTGTAAGGCCTGCAACCTGGGCGAGGGCGT |
| | | GGCGCAGCCGTGCGGGGCCAACCAGACAGTCTGTGA |
| | | GCCGTGCCTAGACAGCGTTACTTTCAGCGATGTGGT |
| | | GAGCGCAACCGAACCATGTAAGCCCTGCACTGAATG |
| | | CGTGGGGTTGCAGTCTATGTCTGCTCCTTGCGTGGAG |
| | | GCCGATGACGCAGTGTGCCGCTGTGCCTACGGCTAC |
| | | TACCAGGACGAGACCACAGGACGATGCGAGGCCTG |
| | | TCGGGTCTGTGAGGCTGGCTCCGGGCTGGTATTCTCC |
| | | TGCCAGGACAAACAGAACACGGTTTGTGAGGAGTGC |
| | | CCCGACGGCACCTACTCTGACGAGGCCAACCACGTA |
| | | GATCCATGCCTGCCCTGCACTGTCTGTGAGGACACG |
| | | GAGCGCCAGCTGAGGGAGTGTACCCGCTGGGCGGA |
| | | CGCCGAGTGCGAGGAGATCCCCGGCCGTTGGATCAC |
| | | CCGCTCTACTCCTCCTGAGGGATCTGACTCCACTGCC |
| | | CCCTCTACTCAGGAGCCAGAGGCTCCCCCCGAACAG |
| | | GACCTGATTGCCTCGACCGTGGCGGGCGTGGTGACC |
| | | ACAGTGATGGGATCTTCACAGCCGGTGGTCACTCGC |
| | | GGAACCACCGACAACCTTATCCCGGTTTACTGCTCT |
| | | ATCCTGGCGGCCGTGGTGGTGGGCTTGGTCGCCTAT |
| | | ATCGCGTTCAAGCGTTGGAACTCCTGA |
| 203 | NOV45 VL-VH AA | MALPVTALLLPLALLLHAARPDILLTQSPATLSLSPGER |
| | | ATLSCRASQNIGTSIQWYQQKPGQAPRLLIRSSSESISGI |
| | | SSRFSGSGSGTDFTLTISSLEPEDFAVYYCQQSNTWPFT |
| | | FGQGTKLEIKGGGGSGGGGSGGGGSGGGGSQVQLVES |
| | | GAEVKKPGASVKVSCKASGYTFTNYIIHWVKQEPGQG |
| | | LEWIGYFNPYNHGTKYNEKFKGRATLTADKSISTAYM |
| | | ELSSLRSEDTAVYYCARSGPYAWFDTWGQGTTVTVSS |
| | | TTTPAPRPPTPAPTIASQPLSLRPEACRPAAGGAVHTRG |
| | | LDFACDIYIWAPLAGTCGVLLLSLVITLYCKRGRKKLL |
| | | YIFKQPFMRPVQTTQEEDGCSCRFPEEEEGGCELRVKF |
| | | SRSADAPAYKQGQNQLYNELNLGRREEYDVLDKRRG |
| | | RDPEMGGKPRRKNPQEGLYNELQKDKMAEAYSEIGM |
| | | KGERRRGKGHDGLYQGLSTATKDTYDALHMQALPPR |
| | | GSGATNFSLLKQAGDVEENPGPMGAGATGRAMDGPR |
| | | LLLLLLLGVSLGGAKEACPTGLYTHSGECCKACNLGE |
| | | GVAQPCGANQTVCEPCLDSVTFSDVVSATEPCKPCTEC |
| | | VGLQSMSAPCVEADDAVCRCAYGYYQDETTGRCEAC |
| | | RVCEAGSGLVFSCQDKQNTVCEECPDGTYSDEANHVD |
| | | PCLPCTVCEDTERQLRECTRWADAECEEIPGRWITRST |
| | | PPEGSDSTAPSTQEPEAPPEQDLIASTVAGVVTTVMGSS |
| | | QPVVTRGTTDNLIPVYCSILAAVVVGLVAYIAFKRWNS |
| | | * |
| 204 | muUCHL1 VH-VL | ATGGCTCTCCCGGTCACCGCTCTGCTGTTGCCCCTGG |
| | | CGTTGTTACTCCACGCCGCGAGGCCTCAAATCACTCT |
| | | AAAGGAGAGCGGACCTGGCATCCTGCAGCCGTCACA |
| | | AACTCTTTCCTTAACCTGCTCCTTCTCGGGCTTCAGC |
| | | TTGACTACCTACGGCATCGGTGTGGGCTGGATCCGC |
| | | CAGCCACCCGGCAAGGGCCTGGAGTGGCTCACCCAC |
| | | ATCTGGTGGAACGACAACAAGTACTACAACACTGCG |
| | | CTCCGCTCTCGCCTGACCATCAGCAAAGACTCCAGC |
| | | AACAACCAGGTCCTGCTGAAGATCGCCAACGTGGAT |
| | | ACCGCCGACACAGCCACTTACTACTGCCTGTACGGC |
| | | TACACCTATTGGGGCCAGGGGACCCTGGTAACGGTG |
| | | TCCGCCGGCGGGGGGGGTCCGGAGGCGGTGGCTCT |
| | | GGCGGTGGTGGCAGTGACGTGGTGATGACCCAGACT |
| | | CCGCTAAGCCTGCCAGTATCCCTGGGGGACCAGGCG |
| | | TCCATATCGTGCCGATCTAGCCAGAGCCTTCTTTACT |
| | | CCAATGGCAACACGTACCTGCACTGGTACCTGCAGA |
| | | AGCCCGGTCAGAGCCCTAAGCTGCTCATTTACAAGT |
| | | TGTCCAACCGCTTTTCTGGGGTGCCCGACAGGTTTTC |
| | | TGGCTCCGGATCCGGCACCGACTTCACCCTGAAGAT |
| | | TTCTCGAGTGGAGGCGGAGGACCTGGGCGTCTATTT |
| | | CTGTTCGCAGTCTACCCACGTTCCCTGGACCTTCGGA |
| | | GGCGGCACCAAGCTGGAGATCAAGACCACAACACC |
| | | CGCCCCTCGCCCCCCTACCCCAGCCCCGACAATTGCT |
| | | TCCCAGCCACTGAGTTTGCGCCCCGAGGCTTGCCGT |
| | | CCTGCCGCGGGTGGCGCGGTGCACACTCGCGGCTTG |
| | | GATTTCGCTTGTGACATCTATATATGGGCTCCCCTGG |
| | | CCGGCACCTGTGGAGTTCTTTTGTTATCCCTGGTGAT |
| | | CACTCTCTACTGCAAGCGCGGACGCAAGAAGCTGTT |
| | | GTACATCTTCAAGCAGCCCTTCATGAGGCCGGTGCA |
| | | GACGACCCAGGAGGAAGACGGGTGCTCATGCCGCTT |
| | | CCCCGAGGAGGAGGAAGGCGGGTGCGAGCTGCGCG |
| | | TGAAATTTTCCCGCAGCGCGGACGCCCCTGCGTACA |
| | | AGCAAGGACAGAACCAGCTGTACAACGAGCTCAAC |
| | | CTGGGCCGCAGGGAGGAGTACGATGTGCTGGACAA |
| | | GCGCCGTGGTCGCGACCCGGAGATGGGGGGGAAGC |
| | | CGCGCCGCAAGAATCCTCAGGAGGGCCTTTACAACG |
| | | AGCTGCAGAAGGACAAAATGGCGGAGGCCTACTCC |
| | | GAGATTGGAATGAAAGGCGAACGGCGTCGTGGCAA |
| | | AGGACATGACGGGCTGTACCAGGGCCTATCGACCGC |
| | | AACAAAGGACACCTACGACGCACTACACATGCAGG |
| | | CCCTGCCCCCGCGCGGCTCTGGCGCCACCAACTTTTC |
| | | CCTGCTGAAGCAGGCTGGCGATGTGGAAGAGAACCC |
| | | CGGGCCGATGGGCGCTGGAGCTACCGGCCGCGCCAT |
| | | GGATGGTCCCCGTCTGCTGCTGCTGCTCCTTCTAGGC |
| | | GTGTCTCTGGGTGGTGCTAAGGAGGCTTGCCCTACC |
| | | GGTCTGTATACGCACAGCGGAGAGTGTTGCAAGGCC |
| | | TGCAACCTGGGGGAGGGGGTGGCGCAGCCGTGCGG |
| | | GGCTAACCAGACTGTCTGTGAACCATGTTTGGATTCT |
| | | GTGACCTTCAGTGACGTGGTGTCCGCAACTGAGCCC |
| | | TGCAAGCCCTGTACGGAGTGCGTCGGCCTTCAGTCC |
| | | ATGTCTGCGCCCTGCGTGGAGGCAGATGACGCTGTC |
| | | TGTAGGTGTGCCTACGGCTATTACCAGGACGAGACC |
| | | ACCGGCCGTTGTGAGGCCTGCCGGGTCTGCGAGGCA |
| | | GGCTCGGGGCTCGTGTTCTCATGCCAGGACAAACAG |
| | | AACACGGTCTGCGAGGAGTGCCCAGACGGTACCTAC |
| | | AGCGATGAAGCCAACCATGTAGATCCTTGCCTGCCC |
| | | TGTACAGTTTGCGAGGACACCGAGCGGCAGCTGCGC |
| | | GAGTGTACCCGGTGGGCGGACGCCGAGTGTGAGGA |
| | | GATCCCGGGTCGATGGATCACCCGCTCGACCCCGCC |
| | | CGAGGGCTCCGACAGCACGGCCCCCTCTACCCAGGA |
| | | GCCAGAAGCTCCTCCTGAACAGGATCTGATTGCCAG |
| | | TACTGTGGCCGGCGTGGTCACCACCGTGATGGGCTC |
| | | CTCCCAGCCAGTGGTCACTAGAGGTACCACCGATAA |
| | | TCTTATCCCCGTGTACTGTTCTATCCTGGCAGCCGTG |
| | | GTGGTTGGCCTGGTGGCTTATATCGCGTTCAAACGCT |
| | | GGAATTCCTGA |
| 205 | muUCHL1 VH-VL | MALPVTALLLPLALLLHAARPQITLKESGPGILQPSQTL |
| | AA | SLTCSFSGFSLTTYGIGVGWIRQPPGKGLEWLTHIWWN |
| | | DNKYYNTALRSRLTISKDSSNNQVLLKIANVDTADTAT |
| | | YYCLYGYTYWGQGTLVTVSAGGGGSGGGGSGGGGSD |
| | | VVMTQTPLSLPVSLGDQASISCRSSQSLLYSNGNTYLH |
| | | WYLQKPGQSPKLLIYKLSNRFSGVPDRFSGSGSGTDFT |
| | | LKISRVEAEDLGVYFCSQSTHVPWTFGGGTKLEIKTTT |
| | | PAPRPPTPAPTIASQPLSLRPEACRPAAGGAVHTRGLDF |
| | | ACDIYIWAPLAGTCGVLLLSLVITLYCKRGRKKLLYIFK |
| | | QPFMRPVQTTQEEDGCSCRFPEEEEGGCELRVKFSRSA |
| | | DAPAYKQGQNQLYNELNLGRREEYDVLDKRRGRDPE |
| | | MGGKPRRKNPQEGLYNELQKDKMAEAYSEIGMKGER |
| | | RRGKGHDGLYQGLSTATKDTYDALHMQALPPRGSGA |
| | | TNFSLLKQAGDVEENPGPMGAGATGRAMDGPRLLLLL |
| | | LLGVSLGGAKEACPTGLYTHSGECCKACNLGEGVAQP |
| | | CGANQTVCEPCLDSVTFSDVVSATEPCKPCTECVGLQS |
| | | MSAPCVEADDAVCRCAYGYYQDETTGRCEACRVCEA |
| | | GSGLVFSCQDKQNTVCEECPDGTYSDEANHVDPCLPC |
| | | TVCEDTERQLRECTRWADAECEEIPGRWITRSTPPEGS |
| | | DSTAPSTQEPEAPPEQDLIASTVAGVVTTVMGSSQPVV |
| | | TRGTTDNLIPVYCSILAAVVVGLVAYIAFKRWNS* |
| 206 | muUCHL1 VL-VH | ATGGCCCTGCCTGTGACAGCCCTGCTGCTGCCTCTGG |
| | | CTCTGCTGCTGCATGCCGCTAGACCCGACGTGGTGA |
| | | TGACCCAGACCCCCCTGAGCCTGCCCGTGAGCCTGG |
| | | GCGACCAGGCCAGCATCAGCTGCAGGAGCAGCCAG |
| | | AGCCTGCTGTACAGCAACGGCAACACCTACCTGCAC |
| | | TGGTACCTGCAGAAGCCCGGCCAGAGCCCCAAGCTG |
| | | CTGATCTACAAGCTGAGCAACAGGTTCAGCGGCGTG |
| | | CCCGACAGGTTCAGCGGCAGCGGCAGCGGCACCGA |
| | | CTTCACCCTGAAGATCAGCAGGGTGGAGGCCGAGGA |
| | | CCTGGGCGTGTACTTCTGCAGCCAGAGCACCCACGT |
| | | GCCCTGGACCTTCGGCGGCGGCACCAAGCTGGAGAT |
| | | CAAGGGTGGCGGTGGCTCGGGCGGTGGTGGGTCGG |
| | | GTGGCGGCGGATCTCAGATCACCCTGAAGGAGAGCG |
| | | GCCCCGGCATCCTGCAGCCCAGCCAGACCCTGAGCC |
| | | TGACCTGCAGCTTCAGCGGCTTCAGCCTGACCACCT |
| | | ACGGCATCGGCGTGGGCTGGATCAGGCAGCCCCCCG |
| | | GCAAGGGCCTGGAGTGGCTGACCCACATCTGGTGGA |
| | | ACGACAACAAGTACTACAACACCGCCCTGAGGAGC |
| | | AGGCTGACCATCAGCAAGGACAGCAGCAACAACCA |
| | | GGTGCTGCTGAAGATCGCCAACGTGGACACCGCCGA |
| | | CACCGCCACCTACTACTGCCTGTACGGCTACACCTA |
| | | CTGGGGCCAGGGCACCCTGGTGACCGTGAGCGCCAC |
| | | GACAACCCCGGCCCCGCGTCCTCCTACCCCCGCCCC |
| | | CACCATTGCAAGCCAGCCACTGTCTCTGCGCCCGGA |
| | | GGCGTGCCGACCCGCTGCAGGTGGTGCCGTGCACAC |
| | | TCGCGGCCTCGATTTCGCTTGCGACATCTACATCTGG |
| | | GCTCCATTGGCCGGGACCTGCGGGGTATTGCTGCTG |
| | | TCCCTGGTGATCACTCTTTACTGCAAGCGCGGCCGG |
| | | AAAAAGCTGCTCTACATCTTCAAGCAACCCTTCATG |
| | | AGGCCTGTCCAGACTACCCAGGAGGAAGACGGCTGT |
| | | TCTTGCCGCTTCCCTGAAGAGGAGGAAGGTGGCTGC |
| | | GAGCTGCGGGTGAAATTTTCCCGCAGTGCGGACGCC |
| | | CCCGCCTACAAGCAGGGCCAGAACCAGCTGTACAAC |
| | | GAGCTCAACCTGGGCCGCCGCGAGGAGTACGACGTG |
| | | CTGGACAAACGGCGCGGCCGTGATCCGGAGATGGGT |
| | | GGCAAGCCTCGTAGAAAGAATCCTCAGGAGGGCCTG |
| | | TACAACGAGCTGCAGAAGGATAAAATGGCCGAGGC |
| | | GTACTCGGAGATCGGGATGAAAGGCGAGCGCCGTA |
| | | GGGGAAAGGGCCATGACGGTCTCTATCAGGGATTGT |
| | | CTACCGCCACGAAGGACACCTATGACGCACTGCATA |
| | | TGCAGGCCCTCCCGCCCCGGGGTTCCGGCGCCACCA |
| | | ACTTTTCACTGCTGAAGCAGGCTGGTGACGTGGAGG |
| | | AGAATCCAGGCCCCATGGGGGCCGGCGCCACCGGG |
| | | CGCGCCATGGATGGACCCCGGCTGTTATTACTGCTG |
| | | CTGCTTGGCGTGTCCCTAGGCGGGGCCAAGGAGGCT |
| | | TGTCCTACCGGGCTCTACACCCACTCGGGGGAATGT |
| | | TGTAAGGCCTGTAACCTGGGCGAGGGCGTGGCGCAG |
| | | CCGTGCGGGGCGAACCAGACGGTCTGTGAGCCATGC |
| | | TTGGACAGTGTGACCTTCTCTGATGTGGTGTCCGCCA |
| | | CCGAGCCTTGTAAGCCGTGCACAGAGTGCGTGGGCC |
| | | TGCAATCCATGAGCGCGCCGTGCGTGGAGGCAGATG |
| | | ACGCTGTCTGCCGCTGTGCTTACGGCTATTACCAGG |
| | | ATGAGACTACCGGCAGGTGTGAGGCTTGCCGCGTAT |
| | | GTGAGGCGGGCTCGGGCCTTGTATTCTCTTGCCAGG |
| | | ACAAGCAGAACACGGTGTGCGAGGAGTGTCCCGAC |
| | | GGCACCTACAGTGACGAGGCCAACCACGTGGACCCC |
| | | TGCCTGCCGTGCACCGTGTGCGAGGACACCGAGCGA |
| | | CAGTTGCGCGAGTGCACTCGTTGGGCTGACGCGGAA |
| | | TGTGAGGAGATCCCCGGTCGCTGGATCACCCGCTCC |
| | | ACCCCTCCTGAAGGATCCGATTCGACTGCCCCCTCTA |
| | | CCCAGGAGCCCGAGGCTCCCCCTGAGCAGGACCTGA |
| | | TTGCTTCGACTGTCGCTGGCGTTGTCACCACAGTCAT |
| | | GGGCTCATCGCAGCCGGTCGTCACCAGGGGCACGAC |
| | | CGACAACCTGATACCCGTGTACTGCTCTATCCTGGC |
| | | AGCCGTGGTGGTCGGCCTGGTGGCTTACATCGCGTT |
| | | CAAACGCTGGAACTCCTGA |
| 207 | muUCHL1 VL-VH | MALPVTALLLPLALLLHAARPDVVMTQTPLSLPVSLG |
| | AA | DQASISCRSSQSLLYSNGNTYLHWYLQKPGQSPKLLIY |
| | | KLSNRFSGVPDRFSGSGSGTDFTLKISRVEAEDLGVYFC |
| | | SQSTHVPWTFGGGTKLEIKGGGGSGGGGSGGGGSQITL |
| | | KESGPGILQPSQTLSLTCSFSGFSLTTYGIGVGWIRQPPG |
| | | KGLEWLTHIWWNDNKYYNTALRSRLTISKDSSNNQVL |
| | | LKIANVDTADTATYYCLYGYTYWGQGTLVTVSATTTP |
| | | APRPPTPAPTIASQPLSLRPEACRPAAGGAVHTRGLDFA |
| | | CDIYIWAPLAGTCGVLLLSLVITLYCKRGRKKLLYIFKQ |
| | | PFMRPVQTTQEEDGCSCRFPEEEEGGCELRVKFSRSAD |
| | | APAYKQGQNQLYNELNLGRREEYDVLDKRRGRDPEM |
| | | GGKPRRKNPQEGLYNELQKDKMAEAYSEIGMKGERR |
| | | RGKGHDGLYQGLSTATKDTYDALHMQALPPRGSGAT |
| | | NFSLLKQAGDVEENPGPMGAGATGRAMDGPRLLLLLL |
| | | LGVSLGGAKEACPTGLYTHSGECCKACNLGEGVAQPC |
| | | GANQTVCEPCLDSVTFSDVVSATEPCKPCTECVGLQS |
| | | MSAPCVEADDAVCRCAYGYYQDETTGRCEACRVCEA |
| | | GSGLVFSCQDKQNTVCEECPDGTYSDEANHVDPCLPC |
| | | TVCEDTERQLRECTRWADAECEEIPGRWITRSTPPEGS |
| | | DSTAPSTQEPEAPPEQDLIASTVAGVVTTVMGSSQPVV |
| | | TRGTTDNLIPVYCSILAAVVVGLVAYIAFKRWNS* |
| 208 | huUCHL-1 VH-VL | ATGGCTCTGCCAGTGACGGCACTCTTGCTTCCCCTGG |
| | | CTCTGTTGTTACACGCCGCACGTCCCCAAATCACACT |
| | | CAAGGAGAGCGGCCCCACCCTGGTCAAGCCCACCCA |
| | | GACCCTCACCCTGACCTGTACGTTCTCTGGCTTCTCC |
| | | TTGACAACCTACGGCATCGGCGTGGGCTGGATCCGC |
| | | CAGCCACCCGGCAAGGCTCTGGAGTGGCTCACGCAC |
| | | ATCTGGTGGAACGACAACAAGTACTACTCCCCGAGT |
| | | CTCAAAAGCCGTCTTACCATCACCAAGGATTCCTCT |
| | | AAAAACCAGGTGGTATTGACCATGACAAATATGGAC |
| | | CCGGTAGACACCGCCACTTATTACTGTCTGTACGGCT |
| | | ACACCTATTGGGGCCAGGGGACACTGGTCACCGTCA |
| | | GTGCAGGAGGGGGAGGTAGCGGAGGCGGTGGCTCC |
| | | GGAGGCGGTGGTAGCGATGTGGTAATGACCCAGACT |
| | | CCTTTGTCGCTGCCCGTTACCCTGGGTCAGCCAGCGA |
| | | GCATCTCATGTAGGAGCTCCCAGTCCCTGCTATACA |
| | | GCAACGGCAACACCTACCTGCATTGGTACCAACAGC |
| | | GTCCCGGGCAGTCCCCCCGGCTGCTTATCTACAAGC |
| | | TCAGCAACCGCTTTTCTGGCGTTCCTGACCGCTTCTC |
| | | CGGATCTGGCTCCGGCACCGACTTCACCCTGAAGAT |
| | | TTCACGCGTGGAGGCGGAGGACGTGGGCGTGTACTA |
| | | CTGTTCTCAATCCACCCACGTCCCGTGGACCTTCGGC |
| | | GGCGGCACCAAGCTGGAGATTAAGACCACCACGCCT |
| | | GCCCCGCGCCCCCCAACGCCGGCCCCCACCATCGCC |
| | | TCCCAGCCGCTTTCCCTGCGTCCTGAAGCGTGCCGTC |
| | | CCGCGGCGGGGGGTGCTGTCCACACTCGCGGCCTAG |
| | | ACTTTGCCTGCGACATCTACATCTGGGCCCCTCTAGC |
| | | TGGAACCTGTGGGGTTCTGCTGCTGTCGCTGGTGATT |
| | | ACTCTCTACTGTAAACGCGGTCGCAAGAAGCTGCTC |
| | | TATATCTTCAAACAGCCCTTCATGCGACCCGTGCAA |
| | | ACAACCCAGGAGGAGGACGGCTGTTCATGCCGATTT |
| | | CCTGAGGAAGAGGAAGGTGGGTGCGAGTTGCGCGT |
| | | GAAATTTTCCCGGAGCGCAGACGCACCCGCCTACAA |
| | | GCAGGGCCAGAACCAGCTGTATAACGAGCTCAACCT |
| | | CGGACGGCGCGAGGAGTACGACGTGCTGGACAAGC |
| | | GCCGCGGGCGAGACCCGGAGATGGGCGGAAAACCC |
| | | AGAAGGAAGAATCCTCAGGAGGGTCTGTACAACGA |
| | | GCTGCAGAAGGACAAGATGGCCGAGGCTTACTCCGA |
| | | GATCGGTATGAAGGGTGAGAGACGCAGGGGTAAGG |
| | | GCCATGACGGCCTGTATCAGGGGCTATCGACCGCTA |
| | | CAAAGGACACCTATGATGCGCTTCACATGCAGGCGC |
| | | TGCCACCCCGCGGCTCGGGGGCCACCAACTTCTCGC |
| | | TGCTGAAGCAGGCCGGTGATGTGGAGGAGAACCCA |
| | | GGCCCCATGGGCGCCGGCGCTACTGGTCGCGCCATG |
| | | GATGGCCCTCGCTTACTGCTGCTGCTTCTGTTGGGCG |
| | | TGTCGCTTGGTGGCGCCAAGGAGGCGTGCCCTACCG |
| | | GACTGTACACCCACTCCGGCGAATGCTGCAAGGCTT |
| | | GCAATCTGGGAGAGGGCGTGGCCCAGCCGTGCGGG |
| | | GCCAACCAGACGGTATGCGAGCCATGCCTGGACTCC |
| | | GTGACTTTCTCCGACGTGGTGAGCGCCACCGAGCCC |
| | | TGTAAACCCTGCACGGAGTGCGTGGGCCTTCAGTCT |
| | | ATGTCTGCACCTTGCGTGGAAGCTGATGACGCGGTG |
| | | TGCCGCTGTGCCTACGGCTACTACCAGGATGAGACC |
| | | ACTGGCCGCTGTGAGGCCTGCCGAGTGTGCGAGGCC |
| | | GGCTCTGGCCTCGTGTTCAGTTGCCAGGATAAGCAG |
| | | AACACAGTCTGTGAGGAGTGCCCGGACGGCACTTAC |
| | | AGCGACGAGGCCAATCACGTCGATCCATGTCTGCCC |
| | | TGCACCGTGTGCGAGGATACTGAGCGCCAGCTGCGC |
| | | GAGTGCACCCGGTGGGCGGACGCGGAATGTGAGGA |
| | | GATCCCTGGTCGTTGGATAACGCGCTCTACCCCGCC |
| | | GGAGGGCAGTGACTCTACTGCTCCGTCTACCCAGGA |
| | | ACCCGAGGCTCCCCCTGAGCAGGACCTGATTGCTTC |
| | | CACTGTGGCTGGGGTCGTGACGACCGTGATGGGGTC |
| | | CTCGCAGCCGGTGGTCACTCGGGGAACAACCGACAA |
| | | CCTGATTCCCGTGTATTGCTCTATCCTGGCCGCCGTG |
| | | GTGGTCGGACTTGTGGCGTACATCGCGTTCAAGCGT |
| | | TGGAACTCGTGA |
| 209 | huUCHL-1 VH-VL | MALPVTALLLPLALLLHAARPQITLKESGPTLVKPTQT |
| | AA | LTLTCTFSGFSLTTYGIGVGWIRQPPGKALEWLTHIWW |
| | | NDNKYYSPSLKSRLTITKDSSKNQVVLTMTNMDPVDT |
| | | ATYYCLYGYTYWGQGTLVTVSAGGGGSGGGGSGGG |
| | | GSDVVMTQTPLSLPVTLGQPASISCRSSQSLLYSNGNT |
| | | YLHWYQQRPGQSPRLLIYKLSNRFSGVPDRFSGSGSGT |
| | | DFTLKISRVEAEDVGVYYCSQSTHVPWTFGGGTKLEIK |
| | | TTTPAPRPPTPAPTIASQPLSLRPEACRPAAGGAVHTRG |
| | | LDFACDIYIWAPLAGTCGVLLLSLVITLYCKRGRKKLL |
| | | YIFKQPFMRPVQTTQEEDGCSCRFPEEEEGGCELRVKF |
| | | SRSADAPAYKQGQNQLYNELNLGRREEYDVLDKRRG |
| | | RDPEMGGKPRRKNPQEGLYNELQKDKMAEAYSEIGM |
| | | KGERRRGKGHDGLYQGLSTATKDTYDALHMQALPPR |
| | | GSGATNFSLLKQAGDVEENPGPMGAGATGRAMDGPR |
| | | LLLLLLLGVSLGGAKEACPTGLYTHSGECCKACNLGE |
| | | GVAQPCGANQTVCEPCLDSVTFSDVVSATEPCKPCTEC |
| | | VGLQSMSAPCVEADDAVCRCAYGYYQDETTGRCEAC |
| | | RVCEAGSGLVFSCQDKQNTVCEECPDGTYSDEANHVD |
| | | PCLPCTVCEDTERQLRECTRWADAECEEIPGRWITRST |
| | | PPEGSDSTAPSTQEPEAPPEQDLIASTVAGVVTTVMGSS |
| | | QPVVTRGTTDNLIPVYCSILAAVVVGLVAYIAFKRWNS |
| | | * |
| 210 | huUCHL-1 VL-VH | ATGGCTCTCCCCGTTACTGCACTGCTGTTACCTTTGG |
| | | CGCTCCTTCTGCACGCCGCTCGCCCCGACGTGGTGAT |
| | | GACCCAGACTCCCCTGAGCCTGCCGGTGACCTTGGG |
| | | TCAGCCAGCTTCCATCTCATGCCGCTCGTCACAGAG |
| | | CCTTCTGTACTCCAATGGCAACACTTACCTGCATTGG |
| | | TACCAGCAGCGCCCTGGACAGAGCCCGCGCCTCCTG |
| | | ATCTACAAGTIGTCCAACCGCTTTTCTGGAGTGCCAG |
| | | ACCGCTTCTCGGGCTCGGGCTCCGGTACTGACTTCAC |
| | | CCTGAAGATCTCTCGCGTGGAGGCAGAGGACGTGGG |
| | | CGTCTACTACTGTAGCCAGAGCACTCACGTCCCGTG |
| | | GACCTTCGGAGGCGGTACAAAGCTGGAGATCAAGG |
| | | GCGGTGGTGGGTCTGGAGGCGGCGGCTCTGGCGGTG |
| | | GCGGCAGCCAAATTACTCTGAAGGAGAGCGGGCCG |
| | | ACCCTAGTGAAGCCCACCCAGACCCTGACGCTAACC |
| | | TGCACGTTCAGCGGCTTCTCTCTAACGACTTACGGCA |
| | | TCGGTGTGGGCTGGATCCGCCAGCCGCCTGGTAAGG |
| | | CTTTGGAATGGCTCACTCACATCTGGTGGAACGACA |
| | | ACAAGTACTACAGTCCATCTCTGAAGTCCCGCCTGA |
| | | CCATCACCAAGGATAGTTCCAAGAACCAGGTCGTAC |
| | | TGACAATGACAAACATGGATCCAGTGGACACCGCCA |
| | | CCTATTACTGCCTGTATGGCTATACCTATTGGGGCCA |
| | | GGGCACTCTGGTGACCGTCTCCGCCACGACTACCCC |
| | | TGCTCCCAGGCCTCCTACTCCCGCGCCGACCATCGC |
| | | AAGTCAGCCACTGTCCCTTCGCCCCGAAGCCTGCAG |
| | | ACCCGCCGCGGGAGGTGCGGTGCACACTAGGGGCCT |
| | | CGACTTTGCGTGCGACATCTATATATGGGCTCCTCTG |
| | | GCCGGCACCTGCGGGGTGCTGCTCTTGTCACTGGTG |
| | | ATTACCCTTTACTGTAAGCGCGGCCGCAAGAAGCTG |
| | | CTCTACATCTTCAAGCAACCCTTCATGCGTCCGGTGC |
| | | AGACGACCCAGGAGGAAGATGGGTGCAGTTGTCGTT |
| | | TCCCCGAAGAGGAGGAAGGAGGGTGCGAGCTCCGT |
| | | GTTAAATTTTCCAGATCTGCGGACGCCCCGGCCTAC |
| | | AAGCAGGGACAGAACCAACTGTACAACGAGCTGAA |
| | | TCTGGGCCGAAGGGAGGAGTACGATGTATTGGACAA |
| | | ACGGCGCGGGCGCGACCCGGAGATGGGCGGCAAGC |
| | | CCCGCCGCAAAAACCCTCAGGAGGGCCTCTACAACG |
| | | AGCTGCAGAAAGACAAGATGGCCGAGGCCTACTCC |
| | | GAGATTGGAATGAAGGGAGAGCGGCGTCGCGGCAA |
| | | AGGCCACGACGGTTTATATCAGGGCCTTTCAACGGC |
| | | GACCAAGGACACTTACGACGCATTACACATGCAGGC |
| | | CCTGCCCCCCCGTGGTAGTGGCGCTACCAACTTTTCC |
| | | CTGCTGAAGCAGGCCGGTGATGTGGAGGAGAATCCA |
| | | GGCCCTATGGGAGCAGGTGCTACCGGCCGCGCCATG |
| | | GATGGCCCGCGCCTTTTGCTACTGCTGCTGCTGGGTG |
| | | TATCCCTGGGTGGTGCGAAGGAGGCTTGCCCAACCG |
| | | GTCTGTACACCCATTCGGGGGAATGTTGCAAAGCGT |
| | | GTAACCTGGGGGAGGGCGTGGCGCAGCCCTGCGGG |
| | | GCCAACCAGACCGTGTGCGAGCCTTGTCTGGACTCC |
| | | GTCACATTCAGCGATGTGGTGTCGGCTACCGAGCCC |
| | | TGCAAGCCTTGCACCGAGTGCGTGGGACTCCAGTCT |
| | | ATGTCTGCACCCTGCGTGGAGGCCGATGACGCTGTC |
| | | TGTCGATGCGCCTACGGCTACTACCAGGACGAGACC |
| | | ACCGGCAGGTGTGAGGCTTGCCGAGTGTGCGAGGCG |
| | | GGGAGCGGCTTGGTGTTCTCCTGTCAGGATAAACAG |
| | | AACACGGTCTGTGAGGAGTGCCCAGACGGCACCTAC |
| | | AGCGATGAGGCTAATCACGTAGACCCGTGCCTGCCT |
| | | TGTACAGTCTGCGAAGACACCGAGCGCCAGCTGCGC |
| | | GAGTGTACACGGTGGGCCGACGCCGAGTGCGAGGA |
| | | GATCCCTGGCCGGTGGATCACCCGCTCCACCCCCCC |
| | | CGAGGGCTCCGACTCGACCGCTCCCTCGACCCAGGA |
| | | GCCAGAGGCTCCCCCGGAACAGGACCTGATTGCCTC |
| | | CACCGTCGCTGGGGTTGTGACGACCGTGATGGGCTC |
| | | CTCCCAGCCCGTGGTCACCCGTGGCACCACCGACAA |
| | | CCTCATCCCGGTGTACTGTTCTATCCTGGCGGCCGTG |
| | | GTGGTGGGGCTGGTCGCGTATATCGCGTTCAAGCGT |
| | | TGGAACAGCTGA |
| 211 | huUCHL-1 VL-VH | MALPVTALLLPLALLLHAARPDVVMTQTPLSLPVTLG |
| | AA | QPASISCRSSQSLLYSNGNTYLHWYQQRPGQSPRLLIY |
| | | KLSNRFSGVPDRFSGSGSGTDFTLKISRVEAEDVGVYY |
| | | CSQSTHVPWTFGGGTKLEIKGGGGSGGGGSGGGGSQI |
| | | TLKESGPTLVKPTQTLTLTCTFSGFSLTTYGIGVGWIRQ |
| | | PPGKALEWLTHIWWNDNKYYSPSLKSRLTITKDSSKN |
| | | QVVLTMTNMDPVDTATYYCLYGYTYWGQGTLVTVS |
| | | ATTTPAPRPPTPAPTIASQPLSLRPEACRPAAGGAVHTR |
| | | GLDFACDIYIWAPLAGTCGVLLLSLVITLYCKRGRKKL |
| | | LYIFKQPFMRPVQTTQEEDGCSCRFPEEEEGGCELRVK |
| | | FSRSADAPAYKQGQNQLYNELNLGRREEYDVLDKRR |
| | | GRDPEMGGKPRRKNPQEGLYNELQKDKMAEAYSEIG |
| | | MKGERRRGKGHDGLYQGLSTATKDTYDALHMQALPP |
| | | RGSGATNFSLLKQAGDVEENPGPMGAGATGRAMDGP |
| | | RLLLLLLLGVSLGGAKEACPTGLYTHSGECCKACNLG |
| | | EGVAQPCGANQTVCEPCLDSVTFSDVVSATEPCKPCTE |
| | | CVGLQSMSAPCVEADDAVCRCAYGYYQDETTGRCEA |
| | | CRVCEAGSGLVFSCQDKQNTVCEECPDGTYSDEANHV |
| | | DPCLPCTVCEDTERQLRECTRWADAECEEIPGR WITRS |
| | | TPPEGSDSTAPSTQEPEAPPEQDLIASTVAGVVTTVMGS |
| | | SQPVVTRGTTDNLIPVYCSILAAVVVGLVAYIAFKRWN |
| | | S* |

TABLE 3
Guide RNA Sequences Targeting PTPRC (CD45)

| SEQ | | |
|---|---|---|
| ID | | |
| NO: | Description: | Sequence: |
| 212 | NW45.1 | CCCCCATGAACGTTACCATT |
| 213 | NW45.2 | TACTTGCGTTAGTAAACTTG |
| 214 | NW45.3 | TGTCAAGTGTAGGCCTCCCA |
| 215 | NW45.4 | GTATTTGTGGCTTAAACTCT |
| | (Hs.Cas9.PTPRC.1.AG) | |
| 216 | NW45.5 | GAGTTTAAGCCACAAATACA |
| | (Hs.Cas9.PTPRC.1.BA) | |
| 217 | NW45.6 | TTAATATTAGATGTGCCACC |
| 218 | NW45.7 | AGCCCAACACCTTCCCCCAC |
| 219 | NW45.8 | TGGCTTAAACTCTTGGCATT |
| 220 | NW45.9 | GTTAATGAGAATGTGGAATG |
| 221 | NW45.10 | CTTCTACAAAAAATAATCTG |
| 222 | NW45.11 | TTTGTAGAGAACAACAAGCA |
| 223 | NW45.12 | ACAACCACTCTGAGCCCTTC |
| | (Hs.Cas9.PTPRC.1.AE) | |

TABLE 4
Base-editor sgRNA Sequences

| SEQ ID | | |
|---|---|---|
| NO: | Description: | Sequence: |
| 224 | BE1 | TGGAATGTGGAAACAATACT |
| 225 | BE2 | CATAATTCATGTACTGCTCC |
| 226 | BE3 | ATATCTCATAATTCATGTAC |
| 227 | BE4 | TTAATGAGAATGTGGAATGT |
| 228 | BE5 | GAAAAATATGCAAACATCAC |
| 229 | BE6 | TTGCATATTTTTCATCTAAA |
| 230 | BE7 | TTTTTCATCTAAAAGAAAAT |
| 231 | BE8 | AAAATATGCAAACATCACTG |
| 232 | BE9 | TTACCACATGTTGGCTTAGA |
| 233 | BE10 | ATTAATGTCTTATCAGGAGC |
| 234 | BE11 | GTTAATGAGAATGTGGAATG |
| 235 | BE12 | AAATATGCAAACATCACTGT |
| 236 | BE13 | CCACATGTTGGCTTAGATGG |
| 237 | BE14 | ATCTAAGCCAACATGTGGTA |
| 238 | BE15 | ACTCCATCTAAGCCAACATG |
| 239 | BE16 | AATGAGGTGCATAACCTTAC |
| 240 | BE17 | AGTACATGAATTATGAGATA |
| 241 | BE18 | TCATTAACATTTAGCTTTGC |
| 242 | BE19 | GCTAAATGTTAATGAGAATG |
| 243 | BE20 | CCTGATAAGACATTAATATT |
| 244 | BE21 | TTCTCATTAACATTTAGCTT |
| 245 | BE22 | CCACATTCTCATTAACATTT |
| 246 | BE23 | TGCACCTCATTGTTTGTGCA |
| 247 | BE24 | CAGGAGCAGTACATGAATTA |
| 248 | BE25 | TAAGACATTAATATTAGATG |
| 249 | BE26 | GCACAAACAATGAGGTGCAT |
| 250 | BE27 | ATGAGAATGTGGAATGTGGA |
| 251 | BE28 | AACAATGAGGTGCATAACCT |
| 252 | BE29 | GATGAAAAATATGCAAACAT |

TABLE 5
Base-editor Sequences:

| SEQ | | |
|---|---|---|
| ID | | |
| NO: | Description: | Sequence: |
| 253 | SpCas9-NG-Abe8e | ATGAAACGGACAGCCGACGGAAGCGAGTTCGAGTCAC |
| | | CAAAGAAGAAGCGGAAAGTCTCTGAGGTGGAGTTTTC |
| | | CCACGAGTACTGGATGAGACATGCCCTGACCCTGGCCA |
| | | AGAGGGCACGGGATGAGAGGGAGGTGCCTGTGGGAGC |
| | | CGTGCTGGTGCTGAACAATAGAGTGATCGGCGAGGGC |
| | | TGGAACAGAGCCATCGGCCTGCACGACCCAACAGCCC |
| | | ATGCCGAAATTATGGCCCTGAGACAGGGCGGCCTGGTC |
| | | ATGCAGAACTACAGACTGATTGACGCCACCCTGTACGT |
| | | GACATTCGAGCCTTGCGTGATGTGCGCCGGCGCCATGA |
| | | TCCACTCTAGGATCGGCCGCGTGGTGTTTGGCGTGAGG |
| | | AACTCAAAAAGAGGCGCCGCAGGCTCCCTGATGAACG |
| | | TGCTGAACTACCCCGGCATGAATCACCGCGTCGAAATT |
| | | ACCGAGGGAATCCTGGCAGATGAATGTGCCGCCCTGCT |
| | | GTGCGATTTCTATCGGATGCCTAGACAGGTGTTCAATG |
| | | CTCAGAAGAAGGCCCAGAGCTCCATCAACTCCGGAGG |
| | | ATCTAGCGGAGGCTCCTCTGGCTCTGAGACACCTGGCA |
| | | CAAGCGAGAGCGCAACACCTGAAAGCAGCGGGGGCAG |
| | | CAGCGGGGGGTCAGACAAGAAGTACAGCATCGGCCTG |
| | | GCCATCGGCACCAACTCTGTGGGCTGGGCCGTGATCAC |
| | | CGACGAGTACAAGGTGCCCAGCAAGAAATTCAAGGTG |
| | | CTGGGCAACACCGACCGGCACAGCATCAAGAAGAACC |
| | | TGATCGGAGCCCTGCTGTTCGACAGCGGCGAAACAGCC |
| | | GAGGCCACCCGGCTGAAGAGAACCGCCAGAAGAAGAT |
| | | ACACCAGACGGAAGAACCGGATCTGCTATCTGCAAGA |
| | | GATCTTCAGCAACGAGATGGCCAAGGTGGACGACAGC |
| | | TTCTTCCACAGACTGGAAGAGTCCTTCCTGGTGGAAGA |
| | | GGATAAGAAGCACGAGCGGCACCCCATCTTCGGCAAC |
| | | ATCGTGGACGAGGTGGCCTACCACGAGAAGTACCCCA |
| | | CCATCTACCACCTGAGAAAGAAACTGGTGGACAGCAC |
| | | CGACAAGGCCGACCTGCGGCTGATCTATCTGGCCCTGG |
| | | CCCACATGATCAAGTTCCGGGGCCACTTCCTGATCGAG |
| | | GGCGACCTGAACCCCGACAACAGCGACGTGGACAAGC |
| | | TGTTCATCCAGCTGGTGCAGACCTACAACCAGCTGTTC |
| | | GAGGAAAACCCCATCAACGCCAGCGGCGTGGACGCCA |
| | | AGGCCATCCTGTCTGCCAGACTGAGCAAGAGCAGACG |
| | | GCTGGAAAATCTGATCGCCCAGCTGCCCGGCGAGAAG |
| | | AAGAATGGCCTGTTCGGAAACCTGATTGCCCTGAGCCT |
| | | GGGCCTGACCCCCAACTTCAAGAGCAACTTCGACCTGG |
| | | CCGAGGATGCCAAACTGCAGCTGAGCAAGGACACCTA |
| | | CGACGACGACCTGGACAACCTGCTGGCCCAGATCGGC |
| | | GACCAGTACGCCGACCTGTTTCTGGCCGCCAAGAACCT |
| | | GTCCGACGCCATCCTGCTGAGCGACATCCTGAGAGTGA |
| | | ACACCGAGATCACCAAGGCCCCCCTGAGCGCCTCTATG |
| | | ATCAAGAGATACGACGAGCACCACCAGGACCTGACCC |
| | | TGCTGAAAGCTCTCGTGCGGCAGCAGCTGCCTGAGAAG |
| | | TACAAAGAGATTTTCTTCGACCAGAGCAAGAACGGCTA |
| | | CGCCGGCTACATTGACGGCGGAGCCAGCCAGGAAGAG |
| | | TTCTACAAGTTCATCAAGCCCATCCTGGAAAAGATGGA |
| | | CGGCACCGAGGAACTGCTCGTGAAGCTGAACAGAGAG |
| | | GACCTGCTGCGGAAGCAGCGGACCTTCGACAACGGCA |
| | | GCATCCCCCACCAGATCCACCTGGGAGAGCTGCACGCC |
| | | ATTCTGCGGCGGCAGGAAGATTTTTACCCATTCCTGAA |
| | | GGACAACCGGGAAAAGATCGAGAAGATCCTGACCTTC |
| | | CGCATCCCCTACTACGTGGGCCCTCTGGCCAGGGGAAA |
| | | CAGCAGATTCGCCTGGATGACCAGAAAGAGCGAGGAA |
| | | ACCATCACCCCCTGGAACTTCGAGGAAGTGGTGGACA |
| | | AGGGCGCTTCCGCCCAGAGCTTCATCGAGCGGATGACC |
| | | AACTTCGATAAGAACCTGCCCAACGAGAAGGTGCTGC |
| | | CCAAGCACAGCCTGCTGTACGAGTACTTCACCGTGTAT |
| | | AACGAGCTGACCAAAGTGAAATACGTGACCGAGGGAA |
| | | TGAGAAAGCCCGCCTTCCTGAGCGGCGAGCAGAAAAA |
| | | GGCCATCGTGGACCTGCTGTTCAAGACCAACCGGAAA |
| | | GTGACCGTGAAGCAGCTGAAAGAGGACTACTTCAAGA |
| | | AAATCGAGTGCTTCGACTCCGTGGAAATCTCCGGCGTG |
| | | GAAGATCGGTTCAACGCCTCCCTGGGCACATACCACGA |
| | | TCTGCTGAAAATTATCAAGGACAAGGACTTCCTGGACA |
| | | ATGAGGAAAACGAGGACATTCTGGAAGATATCGTGCT |
| | | GACCCTGACACTGTTTGAGGACAGAGAGATGATCGAG |
| | | GAACGGCTGAAAACCTATGCCCACCTGTTCGACGACAA |
| | | AGTGATGAAGCAGCTGAAGCGGCGGAGATACACCGGC |
| | | TGGGGCAGGCTGAGCCGGAAGCTGATCAACGGCATCC |
| | | GGGACAAGCAGTCCGGCAAGACAATCCTGGATTTCCTG |
| | | AAGTCCGACGGCTTCGCCAACAGAAACTTCATGCAGCT |
| | | GATCCACGACGACAGCCTGACCTTTAAAGAGGACATCC |
| | | AGAAAGCCCAGGTGTCCGGCCAGGGCGATAGCCTGCA |
| | | CGAGCACATTGCCAATCTGGCCGGCAGCCCCGCCATTA |
| | | AGAAGGGCATCCTGCAGACAGTGAAGGTGGTGGACGA |
| | | GCTCGTGAAAGTGATGGGCCGGCACAAGCCCGAGAAC |
| | | ATCGTGATCGAAATGGCCAGAGAGAACCAGACCACCC |
| | | AGAAGGGACAGAAGAACAGCCGCGAGAGAATGAAGC |
| | | GGATCGAAGAGGGCATCAAAGAGCTGGGCAGCCAGAT |
| | | CCTGAAAGAACACCCCGTGGAAAACACCCAGCTGCAG |
| | | AACGAGAAGCTGTACCTGTACTACCTGCAGAATGGGC |
| | | GGGATATGTACGTGGACCAGGAACTGGACATCAACCG |
| | | GCTGTCCGACTACGATGTGGACCATATCGTGCCTCAGA |
| | | GCTTTCTGAAGGACGACTCCATCGACAACAAGGTGCTG |
| | | ACCAGAAGCGACAAGAACCGGGGCAAGAGCGACAAC |
| | | GTGCCCTCCGAAGAGGTCGTGAAGAAGATGAAGAACT |
| | | ACTGGCGGCAGCTGCTGAACGCCAAGCTGATTACCCAG |
| | | AGAAAGTTCGACAATCTGACCAAGGCCGAGAGAGGCG |
| | | GCCTGAGCGAACTGGATAAGGCCGGCTTCATCAAGAG |
| | | ACAGCTGGTGGAAACCCGGCAGATCACAAAGCACGTG |
| | | GCACAGATCCTGGACTCCCGGATGAACACTAAGTACG |
| | | ACGAGAATGACAAGCTGATCCGGGAAGTGAAAGTGAT |
| | | CACCCTGAAGTCCAAGCTGGTGTCCGATTTCCGGAAGG |
| | | ATTTCCAGTTTTACAAAGTGCGCGAGATCAACAACTAC |
| | | CACCACGCCCACGACGCCTACCTGAACGCCGTCGTGGG |
| | | AACCGCCCTGATCAAAAAGTACCCTAAGCTGGAAAGC |
| | | GAGTTCGTGTACGGCGACTACAAGGTGTACGACGTGCG |
| | | GAAGATGATCGCCAAGAGCGAGCAGGAAATCGGCAAG |
| | | GCTACCGCCAAGTACTTCTTCTACAGCAACATCATGAA |
| | | CTTTTTCAAGACCGAGATTACCCTGGCCAACGGCGAGA |
| | | TCCGGAAGCGGCCTCTGATCGAGACAAACGGCGAAAC |
| | | CGGGGAGATCGTGTGGGATAAGGGCCGGGATTTTGCC |
| | | ACCGTGCGGAAAGTGCTGAGCATGCCCCAAGTGAATA |
| | | TCGTGAAAAAGACCGAGGTGCAGACAGGCGGCTTCAG |
| | | CAAAGAGTCTATCAGGCCCAAGAGGAACAGCGATAAG |
| | | CTGATCGCCAGAAAGAAGGACTGGGACCCTAAGAAGT |
| | | ACGGCGGCTTCGTCAGCCCCACCGTGGCCTATTCTGTG |
| | | CTGGTGGTGGCCAAAGTGGAAAAGGGCAAGTCCAAGA |
| | | AACTGAAGAGTGTGAAAGAGCTGCTGGGGATCACCAT |
| | | CATGGAAAGAAGCAGCTTCGAGAAGAATCCCATCGAC |
| | | TTTCTGGAAGCCAAGGGCTACAAAGAAGTGAAAAAGG |
| | | ACCTGATCATCAAGCTGCCTAAGTACTCCCTGTTCGAG |
| | | CTGGAAAACGGCCGGAAGAGAATGCTGGCCTCTGCCA |
| | | GATTCCTGCAGAAGGGAAACGAACTGGCCCTGCCCTCC |
| | | AAATATGTGAACTTCCTGTACCTGGCCAGCCACTATGA |
| | | GAAGCTGAAGGGCTCCCCCGAGGATAATGAGCAGAAA |
| | | CAGCTGTTTGTGGAACAGCACAAGCACTACCTGGACGA |
| | | GATCATCGAGCAGATCAGCGAGTTCTCCAAGAGAGTG |
| | | ATCCTGGCCGACGCTAATCTGGACAAAGTGCTGTCCGC |
| | | CTACAACAAGCACCGGGATAAGCCCATCAGAGAGCAG |
| | | GCCGAGAATATCATCCACCTGTTTACCCTGACCAATCT |
| | | GGGAGCCCCTAGGGCCTTCAAGTACTTTGACACCACCA |
| | | TCGACCGGAAGGTGTACAGGAGCACCAAAGAGGTGCT |
| | | GGACGCCACCCTGATCCACCAGAGCATCACCGGCCTGT |
| | | ACGAGACACGGATCGACCTGTCTCAGCTGGGAGGTGA |
| | | CTCTGGCGGCTCAAAAAGAACCGCCGACGGCAGCGAA |
| | | TTCGAGCCCAAGAAGAAGAGGAAAGTCTAA |
| 254 | SpCas9-NG-Abe8e | MKRTADGSEFESPKKKRKVSEVEFSHEYWMRHALTLAK |
| | AA | RARDEREVPVGAVLVLNNRVIGEGWNRAIGLHDPTAHAE |
| | | IMALRQGGLVMQNYRLIDATLYVTFEPCVMCAGAMIHSR |
| | | IGRVVFGVRNSKRGAAGSLMNVLNYPGMNHRVEITEGIL |
| | | ADECAALLCDFYRMPRQVFNAQKKAQSSINSGGSSGGSS |
| | | GSETPGTSESATPESSGGSSGGSDKKYSIGLAIGTNSVGW |
| | | AVITDEYKVPSKKFKVLGNTDRHSIKKNLIGALLFDSGET |
| | | AEATRLKRTARRRYTRRKNRICYLQEIFSNEMAKVDDSFF |
| | | HRLEESFLVEEDKKHERHPIFGNIVDEVAYHEKYPTIYHL |
| | | RKKLVDSTDKADLRLIYLALAHMIKFRGHFLIEGDLNPDN |
| | | SDVDKLFIQLVQTYNQLFEENPINASGVDAKAILSARLSK |
| | | SRRLENLIAQLPGEKKNGLFGNLIALSLGLTPNFKSNFDLA |
| | | EDAKLQLSKDTYDDDLDNLLAQIGDQYADLFLAAKNLS |
| | | DAILLSDILRVNTEITKAPLSASMIKRYDEHHQDLTLLKAL |
| | | VRQQLPEKYKEIFFDQSKNGYAGYIDGGASQEEFYKFIKPI |
| | | LEKMDGTEELLVKLNREDLLRKQRTFDNGSIPHQIHLGEL |
| | | HAILRRQEDFYPFLKDNREKIEKILTFRIPYYVGPLARGNS |
| | | RFAWMTRKSEETITPWNFEEVVDKGASAQSFIERMTNFD |
| | | KNLPNEKVLPKHSLLYEYFTVYNELTKVKYVTEGMRKPA |
| | | FLSGEQKKAIVDLLFKTNRKVTVKQLKEDYFKKIECFDSV |
| | | EISGVEDRFNASLGTYHDLLKIIKDKDFLDNEENEDILEDI |
| | | VLTLTLFEDREMIEERLKTYAHLFDDKVMKQLKRRRYTG |
| | | WGRLSRKLINGIRDKQSGKTILDFLKSDGFANRNFMQLIH |
| | | DDSLTFKEDIQKAQVSGQGDSLHEHIANLAGSPAIKKGIL |
| | | QTVKVVDELVKVMGRHKPENIVIEMARENQTTQKGQKN |
| | | SRERMKRIEEGIKELGSQILKEHPVENTQLQNEKLYLYYL |
| | | QNGRDMYVDQELDINRLSDYDVDHIVPQSFLKDDSIDNK |
| | | VLTRSDKNRGKSDNVPSEEVVKKMKNYWRQLLNAKLIT |
| | | QRKFDNLTKAERGGLSELDKAGFIKRQLVETRQITKHVA |
| | | QILDSRMNTKYDENDKLIREVKVITLKSKLVSDFRKDFQF |
| | | YKVREINNYHHAHDAYLNAVVGTALIKKYPKLESEFVYG |
| | | DYKVYDVRKMIAKSEQEIGKATAKYFFYSNIMNFFKTEIT |
| | | LANGEIRKRPLIETNGETGEIVWDKGRDFATVRKVLSMPQ |
| | | VNIVKKTEVQTGGFSKESIRPKRNSDKLIARKKDWDPKK |
| | | YGGFVSPTVAYSVLVVAKVEKGKSKKLKSVKELLGITIM |
| | | ERSSFEKNPIDFLEAKGYKEVKKDLIIKLPKYSLFELENGR |
| | | KRMLASARFLQKGNELALPSKYVNFLYLASHYEKLKGSP |
| | | EDNEQKQLFVEQHKHYLDEIIEQISEFSKRVILADANLDK |
| | | VLSAYNKHRDKPIREQAENIIHLFTLTNLGAPRAFKYFDT |
| | | TIDRKVYRSTKEVLDATLIHQSITGLYETRIDLSQLGGDSG |
| | | GSKRTADGSEFEPKKKRKV* |
| 255 | SpCas9-NRCH- | ATGAAACGGACAGCCGACGGAAGCGAGTTCGAGTCAC |
| | Abe8e | CAAAGAAGAAGCGGAAAGTCTCTGAGGTGGAGTTTTC |
| | | CCACGAGTACTGGATGAGACATGCCCTGACCCTGGCCA |
| | | AGAGGGCACGGGATGAGAGGGAGGTGCCTGTGGGAGC |
| | | CGTGCTGGTGCTGAACAATAGAGTGATCGGCGAGGGC |
| | | TGGAACAGAGCCATCGGCCTGCACGACCCAACAGCCC |
| | | ATGCCGAAATTATGGCCCTGAGACAGGGCGGCCTGGTC |
| | | ATGCAGAACTACAGACTGATTGACGCCACCCTGTACGT |
| | | GACATTCGAGCCTTGCGTGATGTGCGCCGGCGCCATGA |
| | | TCCACTCTAGGATCGGCCGCGTGGTGTTTGGCGTGAGG |
| | | AACTCAAAAAGAGGCGCCGCAGGCTCCCTGATGAACG |
| | | TGCTGAACTACCCCGGCATGAATCACCGCGTCGAAATT |
| | | ACCGAGGGAATCCTGGCAGATGAATGTGCCGCCCTGCT |
| | | GTGCGATTTCTATCGGATGCCTAGACAGGTGTTCAATG |
| | | CTCAGAAGAAGGCCCAGAGCTCCATCAACTCCGGAGG |
| | | ATCTAGCGGAGGCTCCTCTGGCTCTGAGACACCTGGCA |
| | | CAAGCGAGAGCGCAACACCTGAAAGCAGCGGGGGCAG |
| | | CAGCGGGGGGTCAGACAAGAAGTACAGCATCGGCCTG |
| | | ACCATCGGCACCAACTCTGTGGGCTGGGCCGTGATCAC |
| | | CGACGAGTACAAGGTGCCCAGCAAGAAATTCAAGGTG |
| | | CTGGGCAACACCGACCGGCACAGCATCAAGAAGAACC |
| | | TGATCGGAGCCCTGCTGTTCGACAGCGGCGAAACAGCC |
| | | GAGGCCACCCGGCTGAAGAGAACCGCCAGAAGAAGAT |
| | | ACACCAGACGGAAGAACCGGATCTGCTATCTGCAAGA |
| | | GATCTTCAGCAACGAGATGGCCAAGGTGGACGACAGC |
| | | TTCTTCCACAGACTGGAAGAGTCCTTCCTGGTGGAAGA |
| | | GGATAAGAAGCACGAGCGGCACCCCATCTTCGGCAAC |
| | | ATCGTGGACGAGGTGGCCTACCACGAGAAGTACCCCA |
| | | CCATCTACCACCTGAGAAAGAAACTGGTGGACAGCAC |
| | | CGACAAGGCCGACCTGCGGCTGATCTATCTGGCCCTGG |
| | | CCCACATGATCAAGTTCCGGGGCCACTTCCTGATCGAG |
| | | GGCGACCTGAACCCCGACAACAGCGACGTGGACAAGC |
| | | TGTTCATCCAGCTGGTGCAGACCTACAACCAGCTGTTC |
| | | GAGGAAAACCCCATCAACGCCAGCGGCGTGGACGCCA |
| | | AGGCCATCCTGTCTGCCAGACTGAGCAAGAGCAGACG |
| | | GCTGGAAAATCTGATCGCCCAGCTGCCCGGCGAGAAG |
| | | AAGAATGGCCTGTTCGGAAACCTGATTGCCCTGAGCCT |
| | | GGGCCTGACCCCCAACTTCAAGAGCAACTTCGACCTGG |
| | | CCGAGGATGCCAAACTGCAGCTGAGCAAGGACACCTA |
| | | CGACGACGACCTGGACAACCTGCTGGCCCAGATCGGC |
| | | GACCAGTACGCCGACCTGTTTCTGGCCGCCAAGAACCT |
| | | GTCCGACGCCATCCTGCTGAGCGACATCCTGAGAGTGA |
| | | ACACCGAGATCACCAAGGCCCCCCTGAGCGCCTCTATG |
| | | GTGAAGAGATACGACGAGCACCACCAGGACCTGACCC |
| | | TGCTGAAAGCTCTCGTGCGGCAGCAGCTGCCTGAGAAG |
| | | TACAAAGAGATTTTCTTCGACCAGAGCAAGAACGGCTA |
| | | CGCCGGCTACATTGACGGCGGAGCCAGCCAGGAAGAG |
| | | TTCTACAAGTTCATCAAGCCCATCCTGGAAAAGATGGA |
| | | CGGCACCGAGGAACTGCTCGTGAAGCTGAACAGAGAG |
| | | GACCTGCTGCGGAAGCAGCGGACCTTCGACAACGGCA |
| | | TTATCCCCCACCAGATCCACCTGGGAGAGCTGCACGCC |
| | | ATTCTGCGGCGGCAGGGCGATTTTTACCCATTCCTGAA |
| | | GGACAACCGGGAAAAGATCGAGAAGATCCTGACCTTC |
| | | CGCATCCCCTACTACGTGGGCCCTCTGGCCAGGGGAAA |
| | | CAGCAGATTCGCCTGGATGACCAGAAAGAGCGAGGAA |
| | | ACCATCACCCCCTGGAACTTCGAGGAAGTGGTGGACA |
| | | AGGGCGCTTCCGCCCAGAGCTTCATCGAGCGGATGACC |
| | | AACTTCGATAAGAACCTGCCCAACGAGAAGGTGCTGC |
| | | CCAAGCACAGCCTGCTGTACGAGTACTTCACCGTGTAT |
| | | AACGAGCTGACCAAAGTGAAATACGTGACCGAGGGAA |
| | | TGAGAAAGCCCGCCTTCCTGAGCGGCGAGCAGAAAAA |
| | | GGCCATCGTGGACCTGCTGTTCAAGACCAACCGGAAA |
| | | GTGACCGTGAAGCAGCTGAAAGAGGACTACTTCAAGA |
| | | AAATCGAGTGCTTCGACTCCGTGGAAATCTCCGGCGTG |
| | | GAAGATCGGTTCAACGCCTCCCTGGGCACATACCACGA |
| | | TCTGCTGAAAATTATCAAGGACAAGGACTTCCTGGACA |
| | | ATGAGGAAAACGAGGACATTCTGGAAGATATCGTGCT |
| | | GACCCTGACACTGTTTGAGGACAGAGAGATGATCGAG |
| | | GAACGGCTGAAAACCTATGCCCACCTGTTCGACGACAA |
| | | AGTGATGAAGCAGCTGAAGCGGCTGAGATACACCGGC |
| | | TGGGGCAGGCTGAGCCGGAAGCTGATCAACGGCATCC |
| | | GGGACAAGCAGTCCGGCAAGACAATCCTGGATTTCCTG |
| | | AAGTCCGACGGCTTCGCCAACAGAAACTTCATGCAGCT |
| | | GATCCACGACGACAGCCTGACCTTTAAAGAGGACATCC |
| | | AGAAAGCCCAGGTGTCCGGCCAGGGCGATAGCCTGCA |
| | | CGAGCACATTGCCAATCTGGCCGGCAGCCCCGCCATTA |
| | | AGAAGGGCATCCTGCAGACAGTGAAGGTGGTGGACGA |
| | | GCTCGTGAAAGTGATGGGCGGCCACAAGCCCGAGAAC |
| | | ATCGTGATCGAAATGGCCAGAGAGAACCAGACCACCC |
| | | AGAAGGGACAGAAGAACAGCCGCGAGAGAATGAAGC |
| | | GGATCGAAGAGGGCATCAAAGAGCTGGGCAGCCAGAT |
| | | CCTGAAAGAACACCCCGTGGAAAACACCCAGCTGCAG |
| | | AACGAGAAGCTGTACCTGTACTACCTGCAGAATGGGC |
| | | GGGATATGTACGTGGACCAGGAACTGGACATCAACCG |
| | | GCTGTCCGACTACGATGTGGACCATATCGTGCCTCAGA |
| | | GCTTTCTGAAGGACGACTCCATCGACAACAAGGTGCTG |
| | | ACCAGAAGCGACAAGAACCGGGGCAAGAGCGACAAC |
| | | GTGCCCTCCGAAGAGGTCGTGAAGAAGATGAAGAACT |
| | | ACTGGCGGCAGCTGCTGAACGCCAAGCTGATTACCCAG |
| | | AGAAAGTTCGACAATCTGACCAAGGCCGAGAGAGGCG |
| | | GCCTGAGCGAACTGGATAAGGCCGGCTTCATCAAGAG |
| | | ACAGCTGGTGGAAACCCGGCAGATCACAAAGCACGTG |
| | | GCACAGATCCTGGACTCCCGGATGAACACTAAGTACG |
| | | ACGAGAATGACAAGCTGATCCGGGAAGTGAAAGTGAT |
| | | CACCCTGAAGTCCAAGCTGGTGTCCGATTTCCGGAAGG |
| | | ATTTCCAGTTTTACAAAGTGCGCGAGATCAACAACTAC |
| | | CACCACGCCCACGACGCCTACCTGAACGCCGTCGTGGG |
| | | AACCGCCCTGATCAAAAAGTACCCTAAGCTGGAAAGC |
| | | GAGTTCGTGTACGGCGACTACAAGGTGTACGACGTGCG |
| | | GAAGATGATCGCCAAGAGCGAGCAGGAAATCGGCAAG |
| | | GCTACCGCCAAGTACTTCTTCTACAGCAACATCATGAA |
| | | CTTTTTCAAGACCGAGATTACCCTGGCCAACGGCGAGA |
| | | TCCGGAAGCGGCCTCTGATCGAGACAAACGGCGAAAC |
| | | CGGGGAGATCGTGTGGGATAAGGGCCGGGATTTTGCC |
| | | ACCGTGCGGAAAGTGCTGAGCATGCCCCAAGTGAATA |
| | | TCGTGAAAAAGACCGAGGTGCAGACAGGCGGCTTCAG |
| | | CAAAGAGTCTATCCTGCCCAAGGGTAACAGCGATAAG |
| | | CTGATCGCCAGAAAGAAGGACTGGGACCCTAAGAAGT |
| | | ACGGCGGCTTCAACAGCCCCACCGTGGCCTATTCTGTG |
| | | CTGGTGGTGGCCAAAGTGGAAAAGGGCAAGTCCAAGA |
| | | AACTGAAGAGTGTGAAAGAGCTGCTGGGGATCACCAT |
| | | CATGGAAAGAAGCAGCTTCGAGAAGAATCCCATCGAC |
| | | TTTCTGGAAGCCAAGGGCTACAAAGAAGTGAAAAAGG |
| | | ACCTGATCATCAAGCTGCCTAAGTACTCCCTGTTCGAG |
| | | CTGGAAAACGGCCGGAAGAGAATGCTGGCCTCTGCCG |
| | | GCGTGCTGCAGAAGGGAAACGAACTGGCCCTGCCCTC |
| | | CAAATATGTGAACTTCCTGTACCTGGCCAGCCACTATG |
| | | AGAAGCTGAAGGGCTCCCCCGAGGATAATGAGCAGAA |
| | | ACAGCTGTTTGTGGAACAGCACAAGCACTACCTGGACG |
| | | AGATCATCGAGCAGATCAGCGAGTTCTCCAAGAGAGT |
| | | GATCCTGGCCGACGCTAATCTGGACAAAGTGCTGTCCG |
| | | CCTACAACAAGCACCGGGATAAGCCCATCAGAGAGCA |
| | | GGCCGAGAATATCATCCACCTGTTTACCCTGACCAATC |
| | | TGGGAGCCCCTGCCGCCTTCAAGTACTTTGACACCACC |
| | | ATCAACCGGAAGCAATACAACACGACCAAAGAGGTGC |
| | | TGGACGCCACCCTGATCCGTCAGAGCATCACCGGCCTG |
| | | TACGAGACACGGATCGACCTGTCTCAGCTGGGAGGTG |
| | | ACTCTGGCGGCTCAAAAAGAACCGCCGACGGCAGCGA |
| | | ATTCGAGCCCAAGAAGAAGAGGAAAGTCTAA |
| 256 | SpCas9-NRCH- | MKRTADGSEFESPKKKRKVSEVEFSHEYWMRHALTLAK |
| | Abe8e AA | RARDEREVPVGAVLVLNNRVIGEGWNRAIGLHDPTAHAE |
| | | IMALRQGGLVMQNYRLIDATLYVTFEPCVMCAGAMIHSR |
| | | IGRVVFGVRNSKRGAAGSLMNVLNYPGMNHRVEITEGIL |
| | | ADECAALLCDFYRMPRQVFNAQKKAQSSINSGGSSGGSS |
| | | GSETPGTSESATPESSGGSSGGSDKKYSIGLTIGTNSVGWA |
| | | VITDEYKVPSKKFKVLGNTDRHSIKKNLIGALLFDSGETA |
| | | EATRLKRTARRRYTRRKNRICYLQEIFSNEMAKVDDSFFH |
| | | RLEESFLVEEDKKHERHPIFGNIVDEVAYHEKYPTIYHLR |
| | | KKLVDSTDKADLRLIYLALAHMIKFRGHFLIEGDLNPDNS |
| | | DVDKLFIQLVQTYNQLFEENPINASGVDAKAILSARLSKS |
| | | RRLENLIAQLPGEKKNGLFGNLIALSLGLTPNFKSNFDLAE |
| | | DAKLQLSKDTYDDDLDNLLAQIGDQYADLFLAAKNLSD |
| | | AILLSDILRVNTEITKAPLSASMVKRYDEHHQDLTLLKAL |
| | | VRQQLPEKYKEIFFDQSKNGYAGYIDGGASQEEFYKFIKPI |
| | | LEKMDGTEELLVKLNREDLLRKQRTFDNGIIPHQIHLGEL |
| | | HAILRRQGDFYPFLKDNREKIEKILTFRIPYYVGPLARGNS |
| | | RFAWMTRKSEETITPWNFEEVVDKGASAQSFIERMTNFD |
| | | KNLPNEKVLPKHSLLYEYFTVYNELTKVKYVTEGMRKPA |
| | | FLSGEQKKAIVDLLFKTNRKVTVKQLKEDYFKKIECFDSV |
| | | EISGVEDRFNASLGTYHDLLKIIKDKDFLDNEENEDILEDI |
| | | VLTLTLFEDREMIEERLKTYAHLFDDKVMKQLKRLRYTG |
| | | WGRLSRKLINGIRDKQSGKTILDFLKSDGFANRNFMQLIH |
| | | DDSLTFKEDIQKAQVSGQGDSLHEHIANLAGSPAIKKGIL |
| | | QTVKVVDELVKVMGGHKPENIVIEMARENQTTQKGQKN |
| | | SRERMKRIEEGIKELGSQILKEHPVENTQLQNEKLYLYYL |
| | | QNGRDMYVDQELDINRLSDYDVDHIVPQSFLKDDSIDNK |
| | | VLTRSDKNRGKSDNVPSEEVVKKMKNYWRQLLNAKLIT |
| | | QRKFDNLTKAERGGLSELDKAGFIKRQLVETRQITKHVA |
| | | QILDSRMNTKYDENDKLIREVKVITLKSKLVSDFRKDFQF |
| | | YKVREINNYHHAHDAYLNAVVGTALIKKYPKLESEFVYG |
| | | DYKVYDVRKMIAKSEQEIGKATAKYFFYSNIMNFFKTEIT |
| | | LANGEIRKRPLIETNGETGEIVWDKGRDFATVRKVLSMPQ |
| | | VNIVKKTEVQTGGFSKESILPKGNSDKLIARKKDWDPKK |
| | | YGGFNSPTVAYSVLVVAKVEKGKSKKLKSVKELLGITIM |
| | | ERSSFEKNPIDFLEAKGYKEVKKDLIIKLPKYSLFELENGR |
| | | KRMLASAGVLQKGNELALPSKYVNFLYLASHYEKLKGSP |
| | | EDNEQKQLFVEQHKHYLDEIIEQISEFSKRVILADANLDK |
| | | VLSAYNKHRDKPIREQAENIIHLFTLTNLGAPAAFKYFDT |
| | | TINRKQYNTTKEVLDATLIRQSITGLYETRIDLSQLGGDSG |
| | | GSKRTADGSEFEPKKKRKV* |
| 257 | SpCas9-NG- | ATGAAACGGACAGCCGACGGAAGCGAGTTCGAGTCAC |
| | evoAPOBEC- | CAAAGAAGAAGCGGAAAGTCAGTTCAAAGACTGGGCC |
| | BE4max | TGTCGCCGTCGATCCAACCCTGCGCCGCCGGATTGAAC |
| | | CTCACGAGTTTGAAGTGTTCTTTGACCCCCGGGAGCTG |
| | | AGAAAGGAGACATGCCTGCTGTACGAGATCAACTGGG |
| | | GAGGCAGGCACTCCATCTGGAGGCACACCTCTCAGAA |
| | | CACAAATAAGCACGTGGAGGTGAACTTCATCGAGAAG |
| | | TTTACCACAGAGCGGTACTTCTGCCCCAATACCAGATG |
| | | TAGCATCACATGGTTTCTGAGCTGGTCCCCTTGCGGAG |
| | | AGTGTAGCAGGGCCATCACCGAGTTCCTGTCCAGATAT |
| | | CCAAATGTGACACTGTTTATCTACATCGCCAGGCTGTA |
| | | TCACCTGGCAAACCCAAGGAATAGGCAGGGCCTGCGC |
| | | GATCTGATCAGCTCCGGCGTGACCATCCAGATCATGAC |
| | | AGAGCAGGAGTCCGGCTACTGCTGGCACAACTTCGTGA |
| | | ATTATTCTCCTAGCAACGAGTCCCACTGGCCTAGGTAC |
| | | CCACACCTGTGGGTGCGCCTGTACGTGCTGGAGCTGTA |
| | | TTGCATCATCCTGGGCCTGCCCCCTTGTCTGAATATCCT |
| | | GCGGAGAAAGCAGAGCCAGCTGACCTCCTTTACAATC |
| | | GCCCTGCAGTCTTGTCACTATCAGAGGCTGCCACCCCA |
| | | CATCCTGTGGGCCACAGGCCTGAAGTCTGGCGGATCTA |
| | | GCGGAGGATCCTCTGGCAGCGAGACACCAGGAACAAG |
| | | CGAGTCAGCAACACCAGAGAGCAGTGGCGGCAGCAGC |
| | | GGCGGCAGCGACAAGAAGTACAGCATCGGCCTGGCCA |
| | | TCGGCACCAACTCTGTGGGCTGGGCCGTGATCACCGAC |
| | | GAGTACAAGGTGCCCAGCAAGAAATTCAAGGTGCTGG |
| | | GCAACACCGACCGGCACAGCATCAAGAAGAACCTGAT |
| | | CGGAGCCCTGCTGTTCGACAGCGGCGAAACAGCCGAG |
| | | GCCACCCGGCTGAAGAGAACCGCCAGAAGAAGATACA |
| | | CCAGACGGAAGAACCGGATCTGCTATCTGCAAGAGAT |
| | | CTTCAGCAACGAGATGGCCAAGGTGGACGACAGCTTCT |
| | | TCCACAGACTGGAAGAGTCCTTCCTGGTGGAAGAGGAT |
| | | AAGAAGCACGAGCGGCACCCCATCTTCGGCAACATCG |
| | | TGGACGAGGTGGCCTACCACGAGAAGTACCCCACCAT |
| | | CTACCACCTGAGAAAGAAACTGGTGGACAGCACCGAC |
| | | AAGGCCGACCTGCGGCTGATCTATCTGGCCCTGGCCCA |
| | | CATGATCAAGTTCCGGGGCCACTTCCTGATCGAGGGCG |
| | | ACCTGAACCCCGACAACAGCGACGTGGACAAGCTGTT |
| | | CATCCAGCTGGTGCAGACCTACAACCAGCTGTTCGAGG |
| | | AAAACCCCATCAACGCCAGCGGCGTGGACGCCAAGGC |
| | | CATCCTGTCTGCCAGACTGAGCAAGAGCAGACGGCTG |
| | | GAAAATCTGATCGCCCAGCTGCCCGGCGAGAAGAAGA |
| | | ATGGCCTGTTCGGAAACCTGATTGCCCTGAGCCTGGGC |
| | | CTGACCCCCAACTTCAAGAGCAACTTCGACCTGGCCGA |
| | | GGATGCCAAACTGCAGCTGAGCAAGGACACCTACGAC |
| | | GACGACCTGGACAACCTGCTGGCCCAGATCGGCGACC |
| | | AGTACGCCGACCTGTTTCTGGCCGCCAAGAACCTGTCC |
| | | GACGCCATCCTGCTGAGCGACATCCTGAGAGTGAACAC |
| | | CGAGATCACCAAGGCCCCCCTGAGCGCCTCTATGATCA |
| | | AGAGATACGACGAGCACCACCAGGACCTGACCCTGCT |
| | | GAAAGCTCTCGTGCGGCAGCAGCTGCCTGAGAAGTAC |
| | | AAAGAGATTTTCTTCGACCAGAGCAAGAACGGCTACG |
| | | CCGGCTACATTGACGGCGGAGCCAGCCAGGAAGAGTT |
| | | CTACAAGTTCATCAAGCCCATCCTGGAAAAGATGGACG |
| | | GCACCGAGGAACTGCTCGTGAAGCTGAACAGAGAGGA |
| | | CCTGCTGCGGAAGCAGCGGACCTTCGACAACGGCAGC |
| | | ATCCCCCACCAGATCCACCTGGGAGAGCTGCACGCCAT |
| | | TCTGCGGCGGCAGGAAGATTTTTACCCATTCCTGAAGG |
| | | ACAACCGGGAAAAGATCGAGAAGATCCTGACCTTCCG |
| | | CATCCCCTACTACGTGGGCCCTCTGGCCAGGGGAAACA |
| | | GCAGATTCGCCTGGATGACCAGAAAGAGCGAGGAAAC |
| | | CATCACCCCCTGGAACTTCGAGGAAGTGGTGGACAAG |
| | | GGCGCTTCCGCCCAGAGCTTCATCGAGCGGATGACCAA |
| | | CTTCGATAAGAACCTGCCCAACGAGAAGGTGCTGCCCA |
| | | AGCACAGCCTGCTGTACGAGTACTTCACCGTGTATAAC |
| | | GAGCTGACCAAAGTGAAATACGTGACCGAGGGAATGA |
| | | GAAAGCCCGCCTTCCTGAGCGGCGAGCAGAAAAAGGC |
| | | CATCGTGGACCTGCTGTTCAAGACCAACCGGAAAGTGA |
| | | CCGTGAAGCAGCTGAAAGAGGACTACTTCAAGAAAAT |
| | | CGAGTGCTTCGACTCCGTGGAAATCTCCGGCGTGGAAG |
| | | ATCGGTTCAACGCCTCCCTGGGCACATACCACGATCTG |
| | | CTGAAAATTATCAAGGACAAGGACTTCCTGGACAATG |
| | | AGGAAAACGAGGACATTCTGGAAGATATCGTGCTGAC |
| | | CCTGACACTGTTTGAGGACAGAGAGATGATCGAGGAA |
| | | CGGCTGAAAACCTATGCCCACCTGTTCGACGACAAAGT |
| | | GATGAAGCAGCTGAAGCGGCGGAGATACACCGGCTGG |
| | | GGCAGGCTGAGCCGGAAGCTGATCAACGGCATCCGGG |
| | | ACAAGCAGTCCGGCAAGACAATCCTGGATTTCCTGAAG |
| | | TCCGACGGCTTCGCCAACAGAAACTTCATGCAGCTGAT |
| | | CCACGACGACAGCCTGACCTTTAAAGAGGACATCCAG |
| | | AAAGCCCAGGTGTCCGGCCAGGGCGATAGCCTGCACG |
| | | AGCACATTGCCAATCTGGCCGGCAGCCCCGCCATTAAG |
| | | AAGGGCATCCTGCAGACAGTGAAGGTGGTGGACGAGC |
| | | TCGTGAAAGTGATGGGCCGGCACAAGCCCGAGAACAT |
| | | CGTGATCGAAATGGCCAGAGAGAACCAGACCACCCAG |
| | | AAGGGACAGAAGAACAGCCGCGAGAGAATGAAGCGG |
| | | ATCGAAGAGGGCATCAAAGAGCTGGGCAGCCAGATCC |
| | | TGAAAGAACACCCCGTGGAAAACACCCAGCTGCAGAA |
| | | CGAGAAGCTGTACCTGTACTACCTGCAGAATGGGCGG |
| | | GATATGTACGTGGACCAGGAACTGGACATCAACCGGC |
| | | TGTCCGACTACGATGTGGACCATATCGTGCCTCAGAGC |
| | | TTTCTGAAGGACGACTCCATCGACAACAAGGTGCTGAC |
| | | CAGAAGCGACAAGAACCGGGGCAAGAGCGACAACGTG |
| | | CCCTCCGAAGAGGTCGTGAAGAAGATGAAGAACTACT |
| | | GGCGGCAGCTGCTGAACGCCAAGCTGATTACCCAGAG |
| | | AAAGTTCGACAATCTGACCAAGGCCGAGAGAGGCGGC |
| | | CTGAGCGAACTGGATAAGGCCGGCTTCATCAAGAGAC |
| | | AGCTGGTGGAAACCCGGCAGATCACAAAGCACGTGGC |
| | | ACAGATCCTGGACTCCCGGATGAACACTAAGTACGAC |
| | | GAGAATGACAAGCTGATCCGGGAAGTGAAAGTGATCA |
| | | CCCTGAAGTCCAAGCTGGTGTCCGATTTCCGGAAGGAT |
| | | TTCCAGTTTTACAAAGTGCGCGAGATCAACAACTACCA |
| | | CCACGCCCACGACGCCTACCTGAACGCCGTCGTGGGA |
| | | ACCGCCCTGATCAAAAAGTACCCTAAGCTGGAAAGCG |
| | | AGTTCGTGTACGGCGACTACAAGGTGTACGACGTGCGG |
| | | AAGATGATCGCCAAGAGCGAGCAGGAAATCGGCAAGG |
| | | CTACCGCCAAGTACTTCTTCTACAGCAACATCATGAAC |
| | | TTTTTCAAGACCGAGATTACCCTGGCCAACGGCGAGAT |
| | | CCGGAAGCGGCCTCTGATCGAGACAAACGGCGAAACC |
| | | GGGGAGATCGTGTGGGATAAGGGCCGGGATTTTGCCA |
| | | CCGTGCGGAAAGTGCTGAGCATGCCCCAAGTGAATATC |
| | | GTGAAAAAGACCGAGGTGCAGACAGGCGGCTTCAGCA |
| | | AAGAGTCTATCCGGCCCAAGAGGAACAGCGATAAGCT |
| | | GATCGCCAGAAAGAAGGACTGGGACCCTAAGAAGTAC |
| | | GGCGGCTTCGTGAGCCCCACCGTGGCCTATTCTGTGCT |
| | | GGTGGTGGCCAAAGTGGAAAAGGGCAAGTCCAAGAAA |
| | | CTGAAGAGTGTGAAAGAGCTGCTGGGGATCACCATCA |
| | | TGGAAAGAAGCAGCTTCGAGAAGAATCCCATCGACTTT |
| | | CTGGAAGCCAAGGGCTACAAAGAAGTGAAAAAGGACC |
| | | TGATCATCAAGCTGCCTAAGTACTCCCTGTTCGAGCTG |
| | | GAAAACGGCCGGAAGAGAATGCTGGCCTCTGCCCGGT |
| | | TCCTGCAGAAGGGAAACGAACTGGCCCTGCCCTCCAA |
| | | ATATGTGAACTTCCTGTACCTGGCCAGCCACTATGAGA |
| | | AGCTGAAGGGCTCCCCCGAGGATAATGAGCAGAAACA |
| | | GCTGTTTGTGGAACAGCACAAGCACTACCTGGACGAG |
| | | ATCATCGAGCAGATCAGCGAGTTCTCCAAGAGAGTGAT |
| | | CCTGGCCGACGCTAATCTGGACAAAGTGCTGTCCGCCT |
| | | ACAACAAGCACCGGGATAAGCCCATCAGAGAGCAGGC |
| | | CGAGAATATCATCCACCTGTTTACCCTGACCAATCTGG |
| | | GAGCCCCTCGGGCCTTCAAGTACTTTGACACCACCATC |
| | | GACCGGAAGGTGTACCGGAGCACCAAAGAGGTGCTGG |
| | | ACGCCACCCTGATCCACCAGAGCATCACCGGCCTGTAC |
| | | GAGACACGGATCGACCTGTCTCAGCTGGGAGGTGACA |
| | | GCGGCGGGAGCGGCGGGAGCGGGGGGAGCACTAATCT |
| | | GAGCGACATCATTGAGAAGGAGACTGGGAAACAGCTG |
| | | GTCATTCAGGAGTCCATCCTGATGCTGCCTGAGGAGGT |
| | | GGAGGAAGTGATCGGCAACAAGCCAGAGTCTGACATC |
| | | CTGGTGCACACCGCCTACGACGAGTCCACAGATGAGA |
| | | ATGTGATGCTGCTGACCTCTGACGCCCCCGAGTATAAG |
| | | CCTTGGGCCCTGGTCATCCAGGATTCTAACGGCGAGAA |
| | | TAAGATCAAGATGCTGAGCGGAGGATCCGGAGGATCT |
| | | GGAGGCAGCACCAACCTGTCTGACATCATCGAGAAGG |
| | | AGACAGGCAAGCAGCTGGTCATCCAGGAGAGCATCCT |
| | | GATGCTGCCCGAAGAAGTCGAAGAAGTGATCGGAAAC |
| | | AAGCCTGAGAGCGATATCCTGGTCCATACCGCCTACGA |
| | | CGAGAGTACCGACGAAAATGTGATGCTGCTGACATCC |
| | | GACGCCCCAGAGTATAAGCCCTGGGCTCTGGTCATCCA |
| | | GGATTCCAACGGAGAGAACAAAATCAAAATGCTGTCT |
| | | GGCGGCTCAAAAAGAACCGCCGACGGCAGCGAATTCG |
| | | AGCCCAAGAAGAAGAGGAAAGTCTAA |
| 258 | SpCas9-NG- | MKRTADGSEFESPKKKRKVSSKTGPVAVDPTLRRRIEPHE |
| | evoAPOBEC- | FEVFFDPRELRKETCLLYEINWGGRHSIWRHTSQNTNKH |
| | BE4max AA | VEVNFIEKFTTERYFCPNTRCSITWFLSWSPCGECSRAITE |
| | | FLSRYPNVTLFIYIARLYHLANPRNRQGLRDLISSGVTIQI |
| | | MTEQESGYCWHNFVNYSPSNESHWPRYPHLWVRLYVLE |
| | | LYCIILGLPPCLNILRRKQSQLTSFTIALQSCHYQRLPPHIL |
| | | WATGLKSGGSSGGSSGSETPGTSESATPESSGGSSGGSDK |
| | | KYSIGLAIGTNSVGWAVITDEYKVPSKKFKVLGNTDRHSI |
| | | KKNLIGALLFDSGETAEATRLKRTARRRYTRRKNRICYLQ |
| | | EIFSNEMAKVDDSFFHRLEESFLVEEDKKHERHPIFGNIVD |
| | | EVAYHEKYPTIYHLRKKLVDSTDKADLRLIYLALAHMIK |
| | | FRGHFLIEGDLNPDNSDVDKLFIQLVQTYNQLFEENPINAS |
| | | GVDAKAILSARLSKSRRLENLIAQLPGEKKNGLFGNLIAL |
| | | SLGLTPNFKSNFDLAEDAKLQLSKDTYDDDLDNLLAQIG |
| | | DQYADLFLAAKNLSDAILLSDILRVNTEITKAPLSASMIKR |
| | | YDEHHQDLTLLKALVRQQLPEKYKEIFFDQSKNGYAGYI |
| | | DGGASQEEFYKFIKPILEKMDGTEELLVKLNREDLLRKQR |
| | | TFDNGSIPHQIHLGELHAILRRQEDFYPFLKDNREKIEKILT |
| | | FRIPYYVGPLARGNSRFAWMTRKSEETITPWNFEEVVDK |
| | | GASAQSFIERMTNFDKNLPNEKVLPKHSLLYEYFTVYNEL |
| | | TKVKYVTEGMRKPAFLSGEQKKAIVDLLFKTNRKVTVK |
| | | QLKEDYFKKIECFDSVEISGVEDRFNASLGTYHDLLKIIKD |
| | | KDFLDNEENEDILEDIVLTLTLFEDREMIEERLKTYAHLFD |
| | | DKVMKQLKRRRYTGWGRLSRKLINGIRDKQSGKTILDFL |
| | | KSDGFANRNFMQLIHDDSLTFKEDIQKAQVSGQGDSLHE |
| | | HIANLAGSPAIKKGILQTVKVVDELVKVMGRHKPENIVIE |
| | | MARENQTTQKGQKNSRERMKRIEEGIKELGSQILKEHPVE |
| | | NTQLQNEKLYLYYLQNGRDMYVDQELDINRLSDYDVDH |
| | | IVPQSFLKDDSIDNKVLTRSDKNRGKSDNVPSEEVVKKM |
| | | KNYWRQLLNAKLITQRKFDNLTKAERGGLSELDKAGFIK |
| | | RQLVETRQITKHVAQILDSRMNTKYDENDKLIREVKVITL |
| | | KSKLVSDFRKDFQFYKVREINNYHHAHDAYLNAVVGTA |
| | | LIKKYPKLESEFVYGDYKVYDVRKMIAKSEQEIGKATAK |
| | | YFFYSNIMNFFKTEITLANGEIRKRPLIETNGETGEIVWDK |
| | | GRDFATVRKVLSMPQVNIVKKTEVQTGGFSKESIRPKRNS |
| | | DKLIARKKDWDPKKYGGFVSPTVAYSVLVVAKVEKGKS |
| | | KKLKSVKELLGITIMERSSFEKNPIDFLEAKGYKEVKKDLI |
| | | IKLPKYSLFELENGRKRMLASARFLQKGNELALPSKYVNF |
| | | LYLASHYEKLKGSPEDNEQKQLFVEQHKHYLDEIIEQISEF |
| | | SKRVILADANLDKVLSAYNKHRDKPIREQAENIIHLFTLT |
| | | NLGAPRAFKYFDTTIDRKVYRSTKEVLDATLIHQSITGLY |
| | | ETRIDLSQLGGDSGGSGGSGGSTNLSDIIEKETGKQLVIQE |
| | | SILMLPEEVEEVIGNKPESDILVHTAYDESTDENVMLLTSD |
| | | APEYKPWALVIQDSNGENKIKMLSGGSGGSGGSTNLSDII |
| | | EKETGKQLVIQESILMLPEEVEEVIGNKPESDILVHTAYDE |
| | | STDENVMLLTSDAPEYKPWALVIQDSNGENKIKMLSGGS |
| | | KRTADGSEFEPKKKRKV* |

TABLE 6
gRNA sequences targeting CCR5

| SEQ | | |
|---|---|---|
| ID | | |
| NO: | Name: | Sequence: |
| 296 | R5-9 | TAATCCATCTTGTTCCACCC |
| 297 | CCR5KO_1 | TCAGTTTACACCCGATCCACTGG |
| 298 | CCR5KO_2 | AGTTTACACCCGATCCACTGGGG |
| 299 | CCR5KO_3 | TCATCCTCCTGACAATCGATAGG |
| 300 | CCR5KO_4 | CAATGTGTCAACTCTTGACAGGG |
| 301 | BE_sgRNA1 | TAATAATTGATGTCATAGAT |
| 302 | BE_sgRNA4 | TGACATCAATTATTATACAT |
| 303 | BE_sgRNA2 | CTCCGATGTATAATAATTGA |
| 304 | BE_sgRNA6 | TGTATAATAATTGATGTCAT |
| 305 | BE_sgRNA8 | TTATCAGGATGAGGATGACC |
| 306 | BE_sgRNA9 | CATTAAAGATAGTCATCTTG |
| 307 | BE_sgRNA10 | AAGAAGGTCTTCATTACACC |
| 308 | BE_sgRNAc3 | ACAGCATTTGCAGAAGCGTT |
| 309 | BE_sgRNAp24 | GGGACACAGGGTTAATGTGA |
| 310 | BE_sgRNAp7 | AATCCTGCCACCTATGTATC |
| 311 | BE_sgRNAQ280 | TGCAGGTGACAGAGACTCTT |
| 312 | BE_sgRNAK318 | CCAAACGCTTCTGCAAATGC |
| 313 | BE_sgRNAR509 | AAGTTCAGAAACTACCTCTT |

TABLE 7
gRNA sequences targeting CD33

| SEQ | | |
|---|---|---|
| ID | | |
| NO: | Name: | Sequence: |
| 314 | gRNA1 | AGACATGCCGCTGCTGCTAC |
| 317 | gRNA3 | ACTTACAGGTGACGTTGAGC |
| 320 | gRNA4 | TTCCTACCTGAGCCATCTCC |
| 323 | gRNA5 | CACTCACCTGCCCACAGCAG |

### Sources of Immune Cells

Prior to expansion, a source of immune cells is obtained from a subject for ex vivo manipulation. Sources of target cells for ex vivo manipulation may also include, e.g., autologous or heterologous donor blood, cord blood, or bone marrow. For example, the source of immune cells may be from the subject to be treated with the modified immune cells of the invention, e.g., the subject's blood, the subject's cord blood, or the subject's bone marrow. Non-limiting examples of subjects include humans, dogs, cats, mice, rats, and transgenic species thereof. In certain exemplary embodiments, the subject is a human.

Immune cells can be obtained from a number of sources, including blood, peripheral blood mononuclear cells, bone marrow, lymph node tissue, spleen tissue, umbilical cord, lymph, or lymphoid organs. Immune cells are cells of the immune system, such as cells of the innate or adaptive immunity, e.g., myeloid or lymphoid cells, including lymphocytes, typically T cells and/or NK cells and/or NKT cells. Other exemplary cells include stem cells, such as multipotent and pluripotent stem cells, including induced pluripotent stem cells (iPSCs). In certain aspects, the cells are human cells. With reference to the subject to be treated, the cells may be allogeneic and/or autologous. The cells typically are primary cells, such as those isolated directly from a subject and/or isolated from a subject and frozen.

In certain embodiments, the immune cell is a T cell, e.g., a CD8+ T cell (e.g., a CD8+ naive T cell, central memory T cell, or effector memory T cell), a CD4+ T cell, a natural killer T cell (NKT cells), a regulatory T cell (Treg), a stem cell memory T cell, a lymphoid progenitor cell, a hematopoietic stem cell, a natural killer cell (NK cell), a natural killer T cell (NK cell) or a dendritic cell. In some embodiments, the cells are monocytes or granulocytes, e.g., myeloid cells, macrophages, neutrophils, dendritic cells, mast cells, eosinophils, and/or basophils. In an embodiment, the target cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8+ T cell (e.g., a CD8+ naive T cell, central memory T cell, or effector memory T cell), a CD4+ T cell, a stem cell memory T cell, a lymphoid progenitor cell or a hematopoietic stem cell.

In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In certain embodiments, any number of T cell lines available in the art, may be used.

In some embodiments, the methods include isolating immune cells from the subject, preparing, processing, culturing, and/or engineering them. In some embodiments, preparation of the engineered cells includes one or more culture and/or preparation steps. The cells for engineering as described may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g., transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In certain aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig. In some embodiments, isolation of the cells includes one or more preparation and/or non-affinity-based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in certain aspects, contain lymphocytes, including T cells, monocytes, granulocytes, B cells, other nucleated white blood cells, red blood cells, and/or platelets, and in certain aspects contains cells other than red blood cells and platelets. In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, a washing step is accomplished by tangential flow filtration (TFF) according to the manufacturer's instructions. In certain embodiments, the cells are resuspended in a variety of biocompatible buffers after washing. In certain embodiments, components of a blood cell sample are removed, and the cells directly resuspended in culture media. In some embodiments, the methods include density-based cell separation methods, such as the preparation of white blood cells from peripheral blood by lysing the red blood cells and centrifugation through a Percoll or Ficoll gradient.

In one embodiment, immune cells are obtained from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or another saline solution with or without buffer. In some embodiments, the undesirable components of the apheresis sample may be removed, and the cells directly resuspended in culture media.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in certain aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner. Such separation steps can be based on positive selection, in which the cells having bound the reagents are retained for further use, and/or negative selection, in which the cells having not bound to the antibody or binding partner are retained. In some examples, both fractions are retained for further use. In certain aspects, negative selection can be particularly useful where no antibody is available that specifically identifies a cell type in a heterogeneous population, such that separation is best carried out based on markers expressed by cells other than the desired population. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In certain exemplary embodiments, multiple rounds of separation steps are carried out, where the positively or negatively selected fraction from one step is subjected to another separation step, such as a subsequent positive or negative selection. In certain exemplary embodiments, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some embodiments, one or more of the T cell populations is enriched for or depleted of cells that are positive for (marker+) or express high levels (marker^{high}) of one or more particular markers, such as surface markers, or that are negative for (marker-) or express relatively low levels (marker^{low}) of one or more markers. For example, in certain aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8+ cells or the T cells, e.g., CD3+ cells) are enriched for (i.e., positively selected for) cells that are positive or expressing high surface levels of CD45RO, CCR7, CD28, CD27, CD44, CD127, and/or CD62L and/or depleted of (e.g., negatively selected for) cells that are positive for or express high surface levels of CD45RA. In some embodiments, cells are enriched for or depleted of cells positive or expressing high surface levels of CD122, CD95, CD25, CD27, and/or IL7-Ra (CD127). In certain exemplary embodiments, CD8+ T cells are enriched for cells positive for CD45RO (or negative for CD45RA) and for CD62L. For example, CD3+, CD28+ T cells can be positively selected using CD3/CD28 conjugated magnetic beads (e.g., DYNABEADS® M-450 CD3/CD28 T Cell Expander).

In some embodiments, T cells are separated from a PBMC sample by negative selection of markers expressed on non-T cells, such as B cells, monocytes, or other white blood cells, such as CD14. In certain aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations. In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in certain aspects is particularly robust in such sub-populations. In some embodiments, combining TCM-enriched CD8+ T cells and CD4+ T cells further enhances efficacy.

In some embodiments, memory T cells are present in both CD62L+ and CD62L− subsets of CD8+ peripheral blood lymphocytes. PBMC can be enriched for or depleted of CD62L-CD8+ and/or CD62L+CD8+ fractions, such as using anti-CD8 and anti-CD62L antibodies. In some embodiments, a CD4+ T cell population and/or a CD8+ T population is enriched for central memory (TCM) cells. In some embodiments, the enrichment for central memory T (TCM) cells is based on positive or high surface expression of CD45RO, CD62L, CCR7, CD28, CD3, and/or CD 127; in certain aspects, it is based on negative selection for cells expressing or highly expressing CD45RA and/or granzyme B. In certain aspects, isolation of a CD8+ population enriched for TCM cells is carried out by depletion of cells expressing CD4, CD 14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD 14 and CD45RA, and a positive selection based on CD62L. Such selections in certain aspects are carried out simultaneously and in other aspects are carried out sequentially, in either order. In some embodiments, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

CD4+ T helper cells are sorted into naive, central memory, and effector cells by identifying cell populations that have cell surface antigens. CD4+ lymphocytes can be obtained by standard methods. In some embodiments, naive CD4+ T lymphocytes are CD45RO-, CD45RA+, CD62L+, CD4+ T cells. In some embodiments, central memory CD4+ cells are CD62L+ and CD45RO+. In some embodiments, effector CD4+ cells are CD62L− and CD45RO. In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the stimulating conditions or agents include one or more agent, e.g., ligand, which is capable of activating an intracellular signaling domain of a TCR complex. In certain aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In another embodiment, T cells are isolated from peripheral blood by lysing the red blood cells and depleting the monocytes, for example, by centrifugation through a PERCOLL™ gradient. Alternatively, T cells can be isolated from an umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19, and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. An exemplary method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

In one embodiment, the population of T cells is comprised within cells such as peripheral blood mononuclear cells, cord blood cells, a purified population of T cells, and a T cell line. In another embodiment, peripheral blood mononuclear cells comprise the population of T cells. In yet another embodiment, purified T cells comprise the population of T cells.

### Expansion of Immune Cells

Whether prior to or after modification of cells to express a subject CAR, the cells can be activated and expanded in number using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Publication No. 20060121005. For example, the immune cells of the invention may be expanded by contact with a surface having attached thereto an agent that stimulates a CD3/TCR complex associated signal and a ligand that stimulates a co-stimulatory molecule on the surface of the immune cells. In particular, immune cell populations may be stimulated by contact with an anti-CD3 antibody, or an antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the immune cells, a ligand that binds the accessory molecule is used. For example, immune cells can be contacted with an anti-CD3 antibody and an anti-CD28 antibody, under conditions appropriate for stimulating proliferation of the immune cells. Examples of an anti-CD28 antibody include 9.3, B-T3, XR-CD28 (Diaclone, Besancon, France) and these can be used in the invention, as can other methods and reagents known in the art (see, e.g., ten Berge et al., Transplant Proc. (1998) 30(8): 3975-3977; Haanen et al., J. Exp. Med. (1999) 190(9): 1319-1328; and Garland et al., J. Immunol. Methods (1999) 227(1-2): 53-63).

Expanding the immune cells by the methods disclosed herein can be multiplied by about 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700 fold, 800-fold, 900-fold, 1000-fold, 2000-fold, 3000-fold, 4000-fold, 5000-fold, 6000-fold, 7000-fold, 8000-fold, 9000-fold, 10,000-fold, 100,000-fold, 1,000,000-fold, 10,000,000-fold, or greater, and any and all whole or partial integers therebetween. In one embodiment, the immune cells expand in the range of about 20-fold to about 50-fold.

Following culturing, the immune cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. In certain exemplary embodiments, the level of confluence is 70% or greater before passing the cells to another culture apparatus. In particularly exemplary embodiments, the level of confluence is 90% or greater. A period of time can be any time suitable for the culture of cells in vitro. The immune cell medium may be replaced during the culture of the immune cells at any time. In certain exemplary embodiments, the immune cell medium is replaced about every 2 to 3 days. The immune cells are then harvested from the culture apparatus whereupon the immune cells can be used immediately or cryopreserved to be stored for use at a later time. In one embodiment, the invention includes cryopreserving the expanded immune cells. The cryopreserved immune cells are thawed prior to introducing nucleic acids into the immune cell.

In another embodiment, the method comprises isolating immune cells and expanding the immune cells. In another embodiment, the invention further comprises cryopreserving the immune cells prior to expansion. In yet another embodiment, the cryopreserved immune cells are thawed for electroporation with the RNA encoding the chimeric membrane protein.

Another procedure for ex vivo expansion cells is described in U.S. Pat. No. 5,199,942 (incorporated herein by reference). Expansion, such as described in U.S. Pat. No. 5,199,942 can be an alternative or in addition to other methods of expansion described herein. Briefly, ex vivo culture and expansion of immune cells comprises the addition to the cellular growth factors, such as those described in U.S. Pat. No. 5,199,942, or other factors, such as flt3-L, IL-1, IL-3 and c-kit ligand. In one embodiment, expanding the immune cells comprises culturing the immune cells with a factor selected from the group consisting of flt3-L, IL-1, IL-3 and c-kit ligand.

The culturing step as described herein (contact with agents as described herein or after electroporation) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging. Therefore the number of population doublings of a culture is greater than the passage number. The expansion of cells (i.e., the number of population doublings) during the period between passaging depends on many factors, including but is not limited to the seeding density, substrate, medium, and time between passaging.

In one embodiment, the cells may be cultured for several hours (about 3 hours) to about 14 days or any hourly integer value in between. Conditions appropriate for immune cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-beta, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of immune cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

The medium used to culture the immune cells may include an agent that can co-stimulate the immune cells. For example, an agent that can stimulate CD3 is an antibody to CD3, and an agent that can stimulate CD28 is an antibody to CD28. This is because, as demonstrated by the data disclosed herein, a cell isolated by the methods disclosed herein can be expanded approximately 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, 6000-fold, 7000-fold, 8000-fold, 9000-fold, 10,000-fold, 100,000-fold, 1,000,000-fold, 10,000,000-fold, or greater. In one embodiment, the immune cells expand in the range of about 2-fold to about 50-fold, or more by culturing the electroporated population. In one embodiment, human T regulatory cells are expanded via anti-CD3 antibody coated KT64.86 artificial antigen presenting cells (aAPCs). Methods for expanding and activating immune cells can be found in U.S. Pat. Nos. 7,754,482, 8,722,400, and 9,555,105, the contents of which are incorporated herein in their entirety.

In one embodiment, the method of expanding the immune cells can further comprise isolating the expanded immune cells for further applications. In another embodiment, the method of expanding can further comprise a subsequent electroporation of the expanded immune cells followed by culturing. The subsequent electroporation may include introducing a nucleic acid encoding an agent, such as a transducing the expanded immune cells, transfecting the expanded immune cells, or electroporating the expanded immune cells with a nucleic acid, into the expanded population of immune cells, wherein the agent further stimulates the immune cell. The agent may stimulate the immune cells, such as by stimulating further expansion, effector function, or another immune cell function.

### Methods of Treatment

The modified cells (e.g., T cells) described herein may be included in a composition for immunotherapy. The composition may include a pharmaceutical composition and further include a pharmaceutically acceptable carrier. A therapeutically effective amount of the pharmaceutical composition comprising the modified T cells may be administered.

In one aspect, the invention includes a method for adoptive cell transfer therapy comprising administering to a subject in need thereof a modified T cell of the present invention. In another aspect, the invention includes a method of treating a disease or condition in a subject comprising administering to a subject in need thereof a population of modified T cells.

Also included is a method of treating a disease or condition in a subject in need thereof comprising administering to the subject a genetically edited modified cell (e.g., genetically edited modified T cell). In one embodiment, the method of treating a disease or condition in a subject in need thereof comprises administering to the subject a genetically edited modified cell comprising an exogenous CAR and a modified endogenous CD45 protein.

Also included is a method of treating a cancer in a subject in need thereof comprising administering to the subject a modified immune cell of the current invention, or a modified immune or precursor cell generated by the methods of the current invention and administering to the subject a modified precursor cell comprising a CRISPR-mediated modification in an endogenous gene locus encoding CD45. In some embodiments, the modified precursor cell is selected from the group consisting of a bone marrow stem cell, a hematopoietic progenitor cell, or a cord blood stem cell. In some embodiments, the CRISPR-mediated modification modifies the endogenous CD45 locus in the modified immune cell and modified precursor cell such that the cells expressing the modified CD45 are resistant to recognition by the CD45 CAR. In this way, treatment with the CD45 CAR would deplete the subject of CD45-expressing hematopoietic cells while allowing bone marrow engraftment with bone marrow stem cells comprising the modified CD45 locus. In some embodiments, the modified CD45 protein expressed by the CAR T cells confers resistance to fratricide while maintaining optimal cytotoxic function. In some embodiments, the modified CD45 protein expressed by the modified CD45 protein expressing bone marrow progenitor cells would result in a re-engrafted immune system that is resistant to any remaining CD45 CAR T cells. In certain embodiments, the modified immune cell and modified precursor cell is an autologous cell.

Also included is a method of treating an HIV infection in a subject in need thereof comprising administering to the subject a modified immune cell of the current invention, or a modified immune or precursor cell generated by the methods of the current invention and administering to the subject a modified precursor cell comprising a CRISPR-mediated modification in a first endogenous gene locus and one or more second endogenous gene loci, thereby treating the HIV infection. In some embodiments, the first endogenous gene locus of the precursor cell is CD45 such that the modified immune cell and the modified precursor cell express a modified CD45 protein that is resistant to recognition by the CD45 CAR. In some embodiments, the second endogenous gene loci encodes a receptor that binds to the HIV gp120 protein. HIV gp120 is known to bind to several host-cell surface receptors including, but not limited to, CD4, CCR5, and CXCR4. In some embodiments, any combination of receptor genes may be modified simultaneously. Modification of the receptor proteins is such that the binding site of HIVgp120 is disrupted while maintaining the normal biological function of the receptor protein. In some embodiments, the modification is accomplished using a CRISPR-mediated editing or base-editing system, and the modification is selected from the group consisting of a substitution, an insertion, a deletion, and an insertion/deletion. In certain embodiments, the modified precursor cell is selected from the group consisting of a bone marrow stem cell, a hematopoietic progenitor cell, and a cord blood stem cell. In certain embodiments, the subject is conditioned by pre-treatment with radiation, chemotherapy, or a combination thereof. These treatments, in combination with the CD45 CAR of the invention, serve to efficiently deplete the subject's endogenous hematopoietic cells prior to re-engraftment with the modified precursor cells of the invention. In some embodiments, the conditioning and treatment with CD45 CAR T cells also eliminates endogenous cells which are infected with HIV and act as latent reservoirs of virus and contribute to treatment failure and the need for chronic, life-long treatment with antiviral medications. In this way, in some embodiments, the method of the current invention eliminates HIV-infected endogenous cells and reconstitutes the subject with blood cells which are resistant to HIV infection, thus preventing relapse. In some embodiments, the chemotherapy is selected from the group consisting of anti-thymocyte globulin, carmustine, bulsulfan, carboplatin, cyclosporin A, clofarabine, cyclophosphamide, etoposide, fludarabine, melphalan, methotrexate, tacrolimus, thiotepa, topotecan, or any combination thereof.

Also included is a method of treating a hematologic malignancy in a subject in need thereof, the method comprising administering to the subject a CD33-targeted therapy comprising a CD33-specific CAR-T cell; and administering to the subject a population of modified precursor cells comprising a modification in an endogenous gene locus such that the precursor cells are resistant to the CD33-targeted therapy. Any CAR construct known to bind CD33 may be used in the invention. In one embodiment, a CD33-binding CAR is described in, e.g., PCT publication WO2016/014576, the contents of which are incorporated herein in their entirety. In certain embodiments, the endogenous gene locus is CD33, and the modification is accomplished by a CRISPR-mediated modification. Such modification can include a substitution, an insertion, a deletion, and an insertion/deletion which interrupts transcription or translation of RNA encoding CD33 protein. By way of non-limiting examples, such modifications can alter the transcription start codon, such that initiation of transcription is lost. In another non-limiting example, such modifications can remove splice donor/acceptor sites in any exon of the gene encoding CD33 such that the resulting altered mRNA results in a non-functional protein via exon skipping, or is degraded by natural cell process, for example nonsense-mediated decay, or introduces a non-synonymous amino acid substitution that abrogates recognition of an antibody or scFv-binding site in the CD33 extracellular domain without interrupting the expression or function of CD33.

In certain embodiments, the modified precursor cell is selected from the group consisting of a bone marrow stem cell, a hematopoietic progenitor cell, and a cord blood stem cell.

In certain embodiments, the hematologic malignancy is a myeloid malignancy. Examples of such malignancies includes, but is not limited to, acute myelogenous leukemia (AML), chronic myelogenous leukemia (CML), and myelodysplastic or myeloproliferative neoplasms. It is anticipated that the method of the invention can be used to treat any malignancy associated with the aberrant expression of CD33 by the cancer or malignant cells.

In certain embodiments, these treatments, in combination with the CD33 CAR, serve to efficiently deplete the subject's CD33-expressing cancer cells prior to re-engraftment with the modified precursor cells of the invention. In certain embodiments, the CD33 CAR T cell treatment is combined with other bone marrow-depleting regimens, including the CD45 CAR T cells of the invention, as well as chemotherapy, radiation, or any combination thereof in order to deplete the subject's endogenous hematopoietic progenitor cells prior to engraftment with the CD33 modified HSCs. In this way, in some embodiments, the method of the current invention eliminates CD33-expressing endogenous cells and reconstitutes the subject with blood cells which are resistant to any remaining CD33 CAR T cells.

In certain embodiments, the subject is conditioned by pre-treatment with radiation. In certain embodiments, the subject is conditioned by pre-treatment with chemotherapy. In certain embodiments, the subject is conditioned by a combination of chemotherapy and radiation. In certain embodiments, the subject is conditioned by administering the CD45 CAR T cells of the invention. The purpose of these treatments is to deplete the endogenous, wildtype CD45-expressing hematopoietic cells in the subject prior to administration of the modified CD45-expressing bone marrow precursor or stem cells. A number of chemotherapies are commonly used in such conditioning regiments, typically in advance of a stem cell or cord blood transplant. Non-exclusive examples of such drugs include anti-thymocyte globulin (ATG), carmustine, bulsulfan, carboplatin, cyclosporin A, clofarabine, cyclophosphamide, etoposide, fludarabine, melphalan, methotrexate, tacrolimus, thiotepa, topotecan, among others.

In certain embodiments, the cancer to be treated in a subject in need thereof is a hematologic cancer. As such, in certain embodiments, the invention includes a method of treating a CD45-associated cancer in a subject comprising administering to a subject in need thereof a therapeutically effective population of modified immune cells of the present invention. In some embodiments, the CD45-associated cancer is selected from the group consisting of multiple myeloma, B-cell lymphoma, B-cell leukemia, T cell lymphoma, T cell leukemia, acute myeloid leukemia. In certain embodiments, the modified precursor cell is administered to the subject first. In certain embodiments, the modified immune cell is administered first, followed by the modified precursor cell. In certain embodiments, the modified immune cell and modified precursor cell are administered concurrently.

In another aspect, the invention includes methods of conditioning a patient prior to bone marrow transplant, comprising administering to the subject an effective amount of a modified T cell and/or a modified immune cell of the invention, or a modified immune or precursor cell generated by the methods of the invention. In certain embodiments, the modified T cell comprises an immune cell, preferably a T cell, modified to express a CD45 CAR and a modified CD45 protein. In this way, the CD45 CAR T cell depletes CD45-expressing hematopoietic cells in the subject, rendering them ready to successfully receive and transplant of bone marrow stem cells or progenitor cells. In certain embodiments, the bone marrow stem cells or progenitor cells express a modified CD45 protein that is resistant to recognition by the CD45 CAR. In certain embodiments, the conditioning further comprises administering an effective amount of a chemotherapy, radiation, or a combination thereof that optimizes the depletion of endogenous hematopoietic cells. In certain embodiments, the chemotherapy, radiation, or combination thereof is administered prior to administration of the modified T cell or modified immune cell. In certain embodiments, the conditioning strategy can be combined with other CD45-targeting strategies, including but not limited to antibodies including mAB, antigen-binding fragments, and the like, bispecific T cell engaging antibodies (BiTEs), antibody-drug conjugates (ADC), and radio-immunoconjugates among others. For a nonlimiting example, these other CD45-targeting strategies can be combined with a transplant of bone marrow stem cells or progenitor cells comprising the modified CD45 locus.

In another aspect, the invention includes methods of depleting hematopoietic-derived cells in a subject in need thereof, comprising administering to the subject an effect amount of a modified T cell and/or a modified immune cell of the invention, or a modified immune or precursor cell generated by the methods of the invention. Such depletion can be used as part of a treatment regimen that requires the depletion of hematopoietic cells, a nonlimiting example of which would be the depletion of HIV-positive cells so as to completely eliminate an HIV infection.

Methods for administration of modified immune cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338. In some embodiments, the cell therapy, e.g., adoptive T cell therapy is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject of the same species. In some embodiments, the first and second subjects are genetically identical. In some embodiments, the first and second subjects are genetically similar. In some embodiments, the second subject expresses the same HLA class or supertype as the first subject.

In some embodiments, the subject has been treated with a therapeutic agent targeting the disease or condition, e.g. the tumor, prior to administration of the cells or composition containing the cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy. In certain embodiments, the hematopoietic stem cell transplant comprises hematopoietic stem cells which have been modified to express a modified locus that is resistant to recognition by the modified immune cells of the invention. In certain embodiments, the locus is CD45 gene.

In certain embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at a high risk of relapse, and thus the cells are administered prophylactically, e.g., to reduce the likelihood of or prevent relapse. In some aspects, the subject has not received prior treatment with another therapeutic agent.

In certain embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

The modified immune and modified precursor cells of the present invention can be administered to an animal, preferably a mammal, even more preferably a human, to treat a cancer. In addition, the cells of the present invention can be used for the treatment of any condition related to a cancer, especially a cell-mediated immune response against a tumor cell(s), where it is desirable to treat or alleviate the disease. The types of cancers to be treated with the modified cells or pharmaceutical compositions of the invention include certain leukemia and lymphoid malignancies. Adult tumors/cancers and pediatric tumors/cancers are also included. In one embodiment, the cancer is hematological tumor. In one embodiment, the cancer is a leukemia.

In certain exemplary embodiments, the modified immune cells of the invention are used to treat a myeloma, or a condition related to myeloma. Examples of myeloma or conditions related thereto include, without limitation, light chain myeloma, non-secretory myeloma, monoclonal gammopathy of undetermined significance (MGUS), plasmacytoma (e.g., solitary, multiple solitary, extramedullary plasmacytoma), amyloidosis, and multiple myeloma. In one embodiment, a method of the present disclosure is used to treat multiple myeloma. In one embodiment, a method of the present disclosure is used to treat refractory myeloma. In one embodiment, a method of the present disclosure is used to treat relapsed myeloma.

The cells of the invention to be administered may be autologous, with respect to the subject undergoing therapy.

The administration of the cells of the invention may be carried out in any convenient manner known to those of skill in the art. The cells of the present invention may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells of the invention are injected directly into a site of inflammation in the subject, a local disease site in the subject, a lymph node, an organ, a tumor, and the like.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In certain embodiments, the cells, or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 1×10⁵ cells/kg to about 1×10¹¹ cells/kg 10⁴ and at or about 10¹¹ cells/kilograms (kg) body weight, such as between 10¹ and 10⁶ cells/kg body weight, for example, at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 10⁴ and at or about 109 T cells/kilograms (kg) body weight, such as between 10⁵ and 10⁶ T cells/kg body weight, for example, at or about 1×10⁵ T cells/kg, 1.5×10⁵ T cells/kg, 2×10⁵ T cells/kg, or 1×10⁶ T cells/kg body weight. In other exemplary embodiments, a suitable dosage range of modified cells for use in a method of the present disclosure includes, without limitation, from about 1×10⁵ cells/kg to about 1×10⁶ cells/kg, from about 1×10⁶ cells/kg to about 1×10⁷ cells/kg, from about 1×10⁷ cells/kg about 1×10⁸ cells/kg, from about 1×10⁸ cells/kg about 1×10⁹ cells/kg, from about 1×10⁹ cells/kg about 1×10¹⁰ cells/kg, from about 1×10¹⁰ cells/kg about 1×10¹¹ cells/kg. In an exemplary embodiment, a suitable dosage for use in a method of the present disclosure is about 1×10⁸ cells/kg. In an exemplary embodiment, a suitable dosage for use in a method of the present disclosure is about 1×10⁷ cells/kg. In other embodiments, a suitable dosage is from about 1×10⁷ total cells to about 5×10⁷ total cells. In some embodiments, a suitable dosage is from about 1×10⁸ total cells to about 5×10⁸ total cells. In some embodiments, a suitable dosage is from about 1.4×10⁷ total cells to about 1.1×10⁹ total cells. In an exemplary embodiment, a suitable dosage for use in a method of the present disclosure is about 7×10⁹ total cells.

In some embodiments, the cells are administered at or within a certain range of error of between at or about 10⁴ and at or about 10⁹ CD4⁺ and/or CD8⁺ cells/kilograms (kg) body weight, such as between 10⁵ and 10⁶ CD4⁺ and/or CD8⁺ cells/kg body weight, for example, at or about 1×10⁵ CD4⁺ and/or CD8⁺ cells/kg, 1.5×10⁵ CD4⁺ and/or CD8⁺ cells/kg, 2×10⁵ CD4⁺ and/or CD8⁺ cells/kg, or 1×10⁶ CD4⁺ and/or CD8⁺ cells/kg body weight. In some embodiments, the cells are administered at or within a certain range of error of, greater than, and/or at least about 1×10⁶, about 2.5×10⁶, about 5×10⁶, about 7.5×10⁶, or about 9×10⁶ CD4⁺ cells, and/or at least about 1×10⁶, about 2.5×10⁶, about 5×10⁶, about 7.5×10⁶, or about 9×10⁶ CD8⁺ cells, and/or at least about 1×10⁶, about 2.5×10⁶, about 5×10⁶, about 7.5×10⁶, or about 9×10⁶ T cells. In some embodiments, the cells are administered at or within a certain range of error of between about 108 and 10¹² or between about 10¹⁰ and 10¹¹ T cells, between about 108 and 10¹² or between about 10¹⁰ and 10¹¹ CD4⁺ cells, and/or between about 108 and 10¹² or between about 10¹¹ and 10¹¹ CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios, for example, in some embodiments, the desired ratio (e.g., ratio of CD4+ to CD8+ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In some embodiments, a dose of modified cells is administered to a subject in need thereof, in a single dose or multiple doses. In some embodiments, a dose of modified cells is administered in multiple doses, e.g., once a week or every 7 days, once every 2 weeks or every 14 days, once every 3 weeks or every 21 days, once every 4 weeks or every 28 days. In an exemplary embodiment, a single dose of modified cells is administered to a subject in need thereof. In an exemplary embodiment, a single dose of modified cells is administered to a subject in need thereof by rapid intravenous infusion.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

In certain embodiments, the modified cells of the invention (e.g., a modified cell comprising a CAR) may be administered to a subject in combination with an immune checkpoint antibody (e.g., an anti-PD1, anti-CTLA-4, or anti-PDL1 antibody). For example, the modified cell may be administered in combination with an antibody or antibody fragment targeting, for example, PD-1 (programmed death 1 protein). Examples of anti-PD-1 antibodies include, but are not limited to, pembrolizumab (KEYTRUDA®, formerly lambrolizumab, also known as MK-3475), and nivolumab (BMS-936558, MDX-1106, ONO-4538, OPDIVA®) or an antigen-binding fragment thereof. In certain embodiments, the modified cell may be administered in combination with an anti-PD-L1 antibody or antigen-binding fragment thereof. Examples of anti-PD-Li antibodies include, but are not limited to, BMS-936559, MPDL3280A (TECENTRIQ®, Atezolizumab), and MEDI4736 (Durvalumab, Imfinzi). In certain embodiments, the modified cell may be administered in combination with an anti-CTLA-4 antibody or antigen-binding fragment thereof. An example of an anti-CTLA-4 antibody includes, but is not limited to, Ipilimumab (trade name Yervoy). Other types of immune checkpoint modulators may also be used including, but not limited to, small molecules, siRNA, miRNA, and CRISPR systems. Immune checkpoint modulators may be administered before, after, or concurrently with the modified cell comprising the CAR. In certain embodiments, combination treatment comprising an immune checkpoint modulator may increase the therapeutic efficacy of a therapy comprising a modified cell of the present invention.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD 107a, IFNγ, IL-2, and TNF. In some aspects the biological activity is measured by assessing clinical outcome, such as reduction in tumor burden or load.

In certain embodiments, the subject is provided a secondary treatment. Secondary treatments include but are not limited to chemotherapy, radiation, surgery, and medications.

In some embodiments, the subject can be administered a conditioning therapy prior to CAR T cell therapy. In some embodiments, the conditioning therapy comprises administering an effective amount of cyclophosphamide to the subject. In some embodiments, the conditioning therapy comprises administering an effective amount of fludarabine to the subject. In preferred embodiments, the conditioning therapy comprises administering an effective amount of a combination of cyclophosphamide and fludarabine to the subject. Administration of a conditioning therapy prior to CAR T cell therapy may increase the efficacy of the CAR T cell therapy. Methods of conditioning patients for T cell therapy are described in U.S. Pat. No. 9,855,298, which is incorporated herein by reference in its entirety.

In some embodiments, a specific dosage regimen of the present disclosure includes a lymphodepletion step prior to the administration of the modified T cells. In an exemplary embodiment, the lymphodepletion step includes administration of cyclophosphamide and/or fludarabine.

In some embodiments, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day). In an exemplary embodiment, the dose of cyclophosphamide is about 300 mg/m²/day. In some embodiments, the lymphodepletion step includes administration of fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the dose of fludarabine is about 30 mg/m²/day.

In some embodiment, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day), and fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the lymphodepletion step includes administration of cyclophosphamide at a dose of about 300 mg/m²/day, and fludarabine at a dose of about 30 mg/m²/day.

In an exemplary embodiment, the dosing of cyclophosphamide is 300 mg/m²/day over three days, and the dosing of fludarabine is 30 mg/m²/day over three days.

Dosing of lymphodepletion chemotherapy may be scheduled on Days −6 to −4 (with a −1-day window, i.e., dosing on Days −7 to −5) relative to T cell (e.g., CAR-T, TCR-T, a modified T cell, etc.) infusion on Day 0.

In an exemplary embodiment, for a subject having cancer, the subject receives lymphodepleting chemotherapy including 300 mg/m² of cyclophosphamide by intravenous infusion 3 days prior to administration of the modified T cells. In an exemplary embodiment, for a subject having cancer, the subject receives lymphodepleting chemotherapy including 300 mg/m² of cyclophosphamide by intravenous infusion for 3 days prior to administration of the modified T cells.

In an exemplary embodiment, for a subject having cancer, the subject receives lymphodepleting chemotherapy including fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, for a subject having cancer, the subject receives lymphodepleting chemotherapy including fludarabine at a dose of 30 mg/m² for 3 days.

In an exemplary embodiment, for a subject having cancer, the subject receives lymphodepleting chemotherapy including cyclophosphamide at a dose of between about 200 mg/m²/day and about 2000 mg/m²/day (e.g., 200 mg/m²/day, 300 mg/m²/day, or 500 mg/m²/day), and fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, for a subject having cancer, the subject receives lymphodepleting chemotherapy including cyclophosphamide at a dose of about 300 mg/m²/day, and fludarabine at a dose of 30 mg/m² for 3 days.

Cells of the invention can be administered in dosages and routes and at times to be determined in appropriate pre-clinical and clinical experimentation and trials. Cell compositions may be administered multiple times at dosages within these ranges. Administration of the cells of the invention may be combined with other methods useful to treat the desired disease or condition as determined by those of skill in the art.

It is known in the art that one of the adverse effects following infusion of CAR T cells is the onset of immune activation, known as cytokine release syndrome (CRS). CRS is immune activation resulting in elevated inflammatory cytokines. CRS is a known on-target toxicity, development of which likely correlates with efficacy. Clinical and laboratory measures range from mild CRS (constitutional symptoms and/or grade-2 organ toxicity) to severe CRS (sCRS; grade ≥3 organ toxicity, aggressive clinical intervention, and/or potentially life threatening). Clinical features include high fever, malaise, fatigue, myalgia, nausea, anorexia, tachycardia/hypotension, capillary leak, cardiac dysfunction, renal impairment, hepatic failure, and disseminated intravascular coagulation. Dramatic elevations of cytokines including interferon-gamma, granulocyte macrophage colony-stimulating factor, IL-10, and IL-6 have been shown following CAR T-cell infusion. One CRS signature is elevation of cytokines including IL-6 (severe elevation), IFN-gamma, TNF-alpha (moderate), and IL-2 (mild). Elevations in clinically available markers of inflammation including ferritin and C-reactive protein (CRP) have also been observed to correlate with the CRS syndrome. The presence of CRS generally correlates with expansion and progressive immune activation of adoptively transferred cells. It has been demonstrated that the degree of CRS severity is dictated by disease burden at the time of infusion as patients with high tumor burden experience a more sCRS.

Accordingly, the invention provides for, following the diagnosis of CRS, appropriate CRS management strategies to mitigate the physiological symptoms of uncontrolled inflammation without dampening the antitumor efficacy of the engineered cells (e.g., CAR T cells). CRS management strategies are known in the art. For example, systemic corticosteroids may be administered to rapidly reverse symptoms of sCRS (e.g., grade 3 CRS) without compromising initial antitumor response.

In some embodiments, an anti-IL-6R antibody may be administered. An example of an anti-IL-6R antibody is the Food and Drug Administration-approved monoclonal antibody tocilizumab, also known as atlizumab (marketed as Actemra, or RoActemra). Tocilizumab is a humanized monoclonal antibody against the interleukin-6 receptor (IL-6R). Administration of tocilizumab has demonstrated near-immediate reversal of CRS.

CRS is generally managed based on the severity of the observed syndrome and interventions are tailored as such. CRS management decisions may be based upon clinical signs and symptoms and response to interventions, not solely on laboratory values alone.

Mild to moderate cases generally are treated with symptom management with fluid therapy, non-steroidal anti-inflammatory drug (NSAID) and antihistamines as needed for adequate symptom relief. More severe cases include patients with any degree of hemodynamic instability; with any hemodynamic instability, the administration of tocilizumab is recommended. The first-line management of CRS may be tocilizumab, in some embodiments, at the labeled dose of 8 mg/kg IV over 60 minutes (not to exceed 800 mg/dose); tocilizumab can be repeated Q8 hours. If suboptimal response to the first dose of tocilizumab, additional doses of tocilizumab may be considered. Tocilizumab can be administered alone or in combination with corticosteroid therapy. Patients with continued or progressive CRS symptoms, inadequate clinical improvement in 12-18 hours or poor response to tocilizumab, may be treated with high-dose corticosteroid therapy, generally hydrocortisone 100 mg IV or methylprednisolone 1-2 mg/kg. In patients with more severe hemodynamic instability or more severe respiratory symptoms, patients may be administered high-dose corticosteroid therapy early in the course of the CRS. CRS management guidance may be based on published standards (Lee et al. (2019) Biol Blood Marrow Transplant, doi.org/10.1016/j.bbmt.2018.12.758; Neelapu et al. (2018) Nat Rev Clin Oncology, 15:47; Teachey et al. (2016) Cancer Discov, 6(6):664-679).

Features consistent with Macrophage Activation Syndrome (MAS) or Hemophagocytic lymphohistiocytosis (HLH) have been observed in patients treated with CAR-T therapy (Henter, 2007), coincident with clinical manifestations of the CRS. MAS appears to be a reaction to immune activation that occurs from the CRS, and should therefore be considered a manifestation of CRS. MAS is similar to HLH (also a reaction to immune stimulation). The clinical syndrome of MAS is characterized by high grade non-remitting fever, cytopenias affecting at least two of three lineages, and hepatosplenomegaly. It is associated with high serum ferritin, soluble interleukin-2 receptor, and triglycerides, and a decrease of circulating natural killer (NK) activity.

### Sources of Immune Cells

Prior to expansion, a source of immune cells is obtained from a subject for ex vivo manipulation. Sources of target cells for ex vivo manipulation may also include, e.g., autologous or heterologous donor blood, cord blood, or bone marrow. For example the source of immune cells may be from the subject to be treated with the modified immune cells of the invention, e.g., the subject's blood, the subject's cord blood, or the subject's bone marrow. Non-limiting examples of subjects include humans, dogs, cats, mice, rats, and transgenic species thereof. Preferably, the subject is a human.

Immune cells can be obtained from a number of sources, including blood, peripheral blood mononuclear cells, bone marrow, lymph node tissue, spleen tissue, umbilical cord, lymph, or lymphoid organs. Immune cells are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). In some aspects, the cells are human cells. With reference to the subject to be treated, the cells may be allogeneic and/or autologous. The cells typically are primary cells, such as those isolated directly from a subject and/or isolated from a subject and frozen.

In certain embodiments, the immune cell is a T cell, e.g., a CD8+ T cell (e.g., a CD8+ naive T cell, central memory T cell, or effector memory T cell), a CD4+ T cell, a natural killer T cell (NKT cells), a regulatory T cell (Treg), a stem cell memory T cell, a lymphoid progenitor cell a hematopoietic stem cell, a natural killer cell (NK cell) or a dendritic cell. In some embodiments, the cells are monocytes or granulocytes, e.g., myeloid cells, macrophages, neutrophils, dendritic cells, mast cells, eosinophils, and/or basophils. In an embodiment, the target cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8+ T cell (e.g., a CD8+ naive T cell, central memory T cell, or effector memory T cell), a CD4+ T cell, a stem cell memory T cell, a lymphoid progenitor cell or a hematopoietic stem cell.

In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In certain embodiments, any number of T cell lines available in the art, may be used.

In some embodiments, the methods include isolating immune cells from the subject, preparing, processing, culturing, and/or engineering them. In some embodiments, preparation of the engineered cells includes one or more culture and/or preparation steps. The cells for engineering as described may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig. In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets. In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some aspects, a washing step is accomplished by tangential flow filtration (TFF) according to the manufacturer's instructions. In some embodiments, the cells are resuspended in a variety of biocompatible buffers after washing. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient.

In one embodiment, immune cells are obtained cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed, and the cells directly resuspended in culture media.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some embodiments, one or more of the T cell populations is enriched for or depleted of cells that are positive for (marker+) or express high levels (marker^{high}) of one or more particular markers, such as surface markers, or that are negative for (marker −) or express relatively low levels (marker^{low}) of one or more markers. For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8+ cells or the T cells, e.g., CD3+ cells) are enriched for (i.e., positively selected for) cells that are positive or expressing high surface levels of CD45RO, CCR7, CD28, CD27, CD44, CD 127, and/or CD62L and/or depleted of (e.g., negatively selected for) cells that are positive for or express high surface levels of CD45RA. In some embodiments, cells are enriched for or depleted of cells positive or expressing high surface levels of CD 122, CD95, CD25, CD27, and/or IL7-Ra (CD 127). In some examples, CD8+ T cells are enriched for cells positive for CD45RO (or negative for CD45RA) and for CD62L. For example, CD3+, CD28+ T cells can be positively selected using CD3/CD28 conjugated magnetic beads (e.g., DYNABEADS® M-450 CD3/CD28 T Cell Expander).

In some embodiments, T cells are separated from a PBMC sample by negative selection of markers expressed on non-T cells, such as B cells, monocytes, or other white blood cells, such as CD 14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations. In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. In some embodiments, combining TCM-enriched CD8+ T cells and CD4+ T cells further enhances efficacy.

In some embodiments, memory T cells are present in both CD62L+ and CD62L− subsets of CD8+ peripheral blood lymphocytes. PBMC can be enriched for or depleted of CD62L-CD8+ and/or CD62L+CD8+ fractions, such as using anti-CD8 and anti-CD62L antibodies. In some embodiments, a CD4+ T cell population and a CD8+ T cell sub-population, e.g., a sub-population enriched for central memory (TCM) cells. In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD 14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD 14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

CD4+ T helper cells are sorted into naive, central memory, and effector cells by identifying cell populations that have cell surface antigens. CD4+ lymphocytes can be obtained by standard methods. In some embodiments, naive CD4+ T lymphocytes are CD45RO-, CD45RA+, CD62L+, CD4+ T cells. In some embodiments, central memory CD4+ cells are CD62L+ and CD45RO+. In some embodiments, effector CD4+ cells are CD62L− and CD45RO. In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In another embodiment, T cells are isolated from peripheral blood by lysing the red blood cells and depleting the monocytes, for example, by centrifugation through a PERCOLL™ gradient. Alternatively, T cells can be isolated from an umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19, and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

In one embodiment, the population of T cells is comprised within cells such as peripheral blood mononuclear cells, cord blood cells, a purified population of T cells, and a T cell line. In another embodiment, peripheral blood mononuclear cells comprise the population of T cells. In yet another embodiment, purified T cells comprise the population of T cells.

In certain embodiments, T regulatory cells (Tregs) can be isolated from a sample. The sample can include, but is not limited to, umbilical cord blood or peripheral blood. In certain embodiments, the Tregs are isolated by flow-cytometry sorting. The sample can be enriched for Tregs prior to isolation by any means known in the art. The isolated Tregs can be cryopreserved, and/or expanded prior to use. Methods for isolating Tregs are described in U.S. Pat. Nos. 7,754,482, 8,722,400, and 9,555,105, and U.S. patent application Ser. No. 13/639,927, contents of which are incorporated herein in their entirety.

### Expansion of Immune Cells

Whether prior to or after modification of cells to express a CAR, the cells can be activated and expanded in number using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Publication No. 20060121005. For example, the T cells of the invention may be expanded by contact with a surface having attached thereto an agent that stimulates a CD3/TCR complex associated signal and a ligand that stimulates a co-stimulatory molecule on the surface of the T cells. In particular, T cell populations may be stimulated by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, T cells can be contacted with an anti-CD3 antibody and an anti-CD28 antibody, under conditions appropriate for stimulating proliferation of the T cells. Examples of an anti-CD28 antibody include 9.3, B-T3, XR-CD28 (Diaclone, Besancon, France) and these can be used in the invention, as can other methods and reagents known in the art (see, e.g., ten Berge et al., Transplant Proc. (1998) 30(8): 3975-3977; Haanen et al., J. Exp. Med. (1999) 190(9): 1319-1328; and Garland et al., J. Immunol. Methods (1999) 227(1-2): 53-63).

Expanding T cells by the methods disclosed herein can be multiplied by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers therebetween. In one embodiment, the T cells expand in the range of about 20 fold to about 50 fold.

Following culturing, the T cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. Preferably, the level of confluence is 70% or greater before passing the cells to another culture apparatus. More preferably, the level of confluence is 90% or greater. A period of time can be any time suitable for the culture of cells in vitro. The T cell medium may be replaced during the culture of the T cells at any time. Preferably, the T cell medium is replaced about every 2 to 3 days. The T cells are then harvested from the culture apparatus whereupon the T cells can be used immediately or cryopreserved to be stored for use at a later time. In one embodiment, the invention includes cryopreserving the expanded T cells. The cryopreserved T cells are thawed prior to introducing nucleic acids into the T cell.

In another embodiment, the method comprises isolating T cells and expanding the T cells. In another embodiment, the invention further comprises cryopreserving the T cells prior to expansion. In yet another embodiment, the cryopreserved T cells are thawed for electroporation with the RNA encoding the chimeric membrane protein.

Another procedure for ex vivo expansion cells is described in U.S. Pat. No. 5,199,942 (incorporated herein by reference). Expansion, such as described in U.S. Pat. No. 5,199,942 can be an alternative or in addition to other methods of expansion described herein. Briefly, ex vivo culture and expansion of T cells comprises the addition to the cellular growth factors, such as those described in U.S. Pat. No. 5,199,942, or other factors, such as flt3-L, IL-1, IL-3 and c-kit ligand. In one embodiment, expanding the T cells comprises culturing the T cells with a factor selected from the group consisting of flt3-L, IL-1, IL-3 and c-kit ligand.

The culturing step as described herein (contact with agents as described herein or after electroporation) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but is not limited to the seeding density, substrate, medium, and time between passaging.

In one embodiment, the cells may be cultured for several hours (about 3 hours) to about 14 days or any hourly integer value in between. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-beta, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

The medium used to culture the T cells may include an agent that can co-stimulate the T cells. For example, an agent that can stimulate CD3 is an antibody to CD3, and an agent that can stimulate CD28 is an antibody to CD28. A cell isolated by the methods disclosed herein can be expanded approximately 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater. In one embodiment, the T cells expand in the range of about 20 fold to about 50 fold, or more. In one embodiment, human T regulatory cells are expanded via anti-CD3 antibody coated KT64.86 artificial antigen presenting cells (aAPCs). Methods for expanding and activating T cells can be found in U.S. Pat. Nos. 7,754,482, 8,722,400, and 9,555,105, contents of which are incorporated herein in their entirety.

In one embodiment, the method of expanding the T cells can further comprise isolating the expanded T cells for further applications. In another embodiment, the method of expanding can further comprise a subsequent electroporation of the expanded T cells followed by culturing. The subsequent electroporation may include introducing a nucleic acid encoding an agent, such as a transducing the expanded T cells, transfecting the expanded T cells, or electroporating the expanded T cells with a nucleic acid, into the expanded population of T cells, wherein the agent further stimulates the T cell. The agent may stimulate the T cells, such as by stimulating further expansion, effector function, or another T cell function.

Pharmaceutical compositions and Formulations

Also provided are populations of immune cells of the invention, compositions containing such cells and/or enriched for such cells, such as in which cells expressing the recombinant receptor make up at least 50%, 60%, 70%, 80%, 90%, 91%, 92%, 93%, 94%, 95%, 96%, 97%, 98%, 99%, or more of the total cells in the composition or cells of a certain type such as T cells or CD8+ or CD4+ cells. Among the compositions are pharmaceutical compositions and formulations for administration, such as for adoptive cell therapy. Also provided are therapeutic methods for administering the cells and compositions to subjects, e.g., patients.

Also provided are compositions including the cells for administration, including pharmaceutical compositions and formulations, such as unit dose form compositions including the number of cells for administration in a given dose or fraction thereof. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection. Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyoi (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present invention has been described with reference to the specific embodiments thereof, it should be understood by those skilled in the art that various changes may be made, and equivalents may be substituted without departing from the true spirit and scope of the invention. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present invention. All such modifications are intended to be within the scope of the claims appended hereto. Having now described certain embodiments in detail, the same will be more clearly understood by reference to the following examples, which are included for purposes of illustration only and are not intended to be limiting.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

### Example 1: CD45 as a Target for CAR Based Immunotherapy

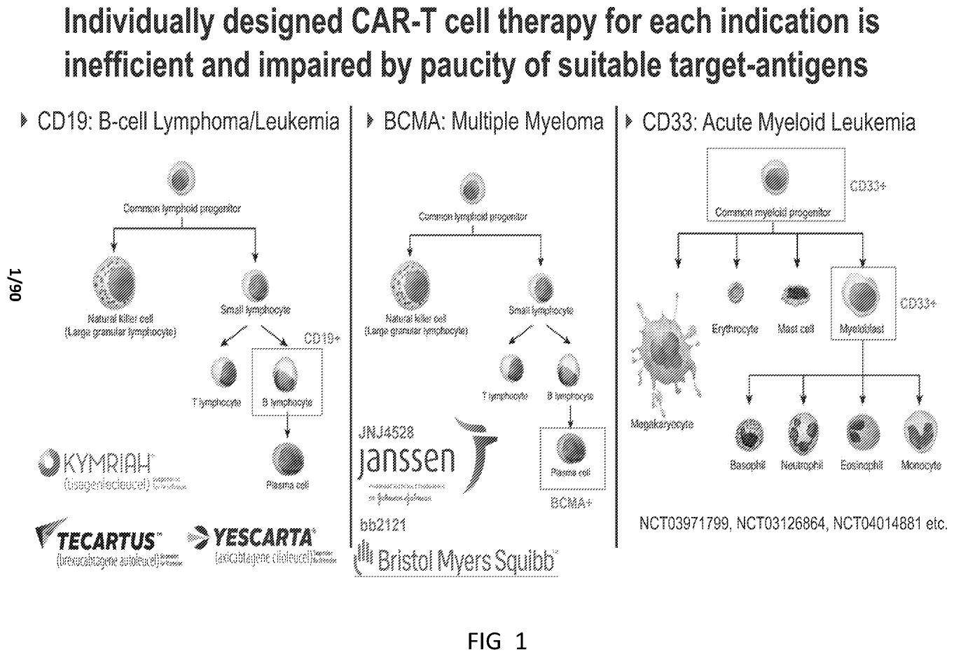

Despite the significant clinical successes of chimeric antigen receptor (CAR)-T cells as a treatment for cancers, especially CD19-expressing B cell lymphomas, certain limitations continue to constrain the wide-spread use of CAR therapy. Foremost among these issues is the simple fact that appropriate cell-surface target antigens must be individually selected and developed for each disease, for example the targeting of lineage-associated antigens such as CD19 for B-cell malignancies or BCMA for myeloma. While this approach has led to the successful treatment of hundreds of patients on clinical trials or with commercial CAR-T cell therapies, from a drug development standpoint this approach is inefficient (FIG. 1). For example, nearly a decade elapsed from the first preclinical publication describing the product that became tisagenlecleucel (CD19 CAR) until its FDA regulatory approval for pediatric B-ALL. Furthermore, targeting lineage associated antigens relies on balancing cytotoxicity directed against the tumor cells with off-target cytotoxicity directed at any normal tissue that also happens to express the target antigen. Again, with the example of CD19 CAR therapy, treatment results in the long-term, effectively permanent, depletion of B cells. This is feasible for B-cell antigens because humans can live without B cells when given supportive care such as i.v. immunoglobulin. For other antigens however, elimination of healthy cells expressing the target antigen (such as CD33) leads to toxicities such as bone marrow depletion. Likewise, this strategy has severe limitations to treat cancers for which no surface marker can be targeted safely.

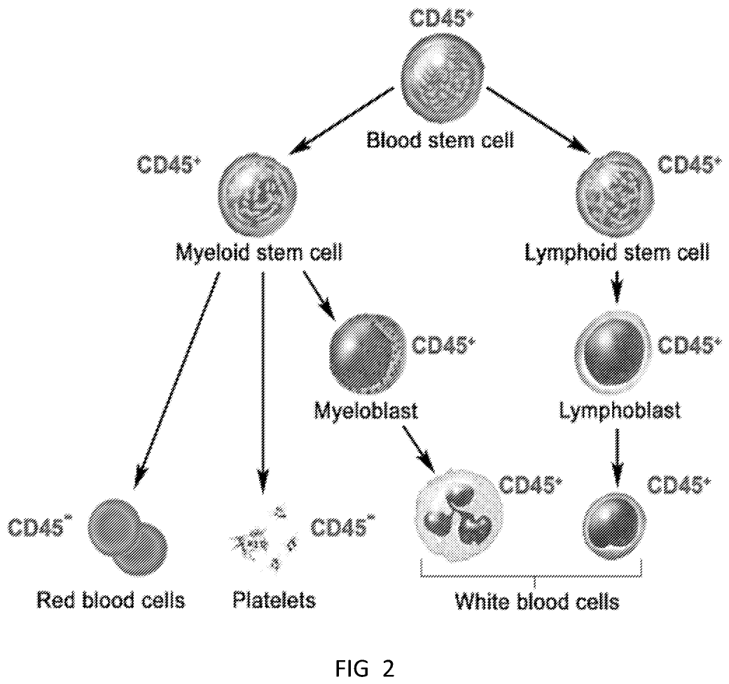

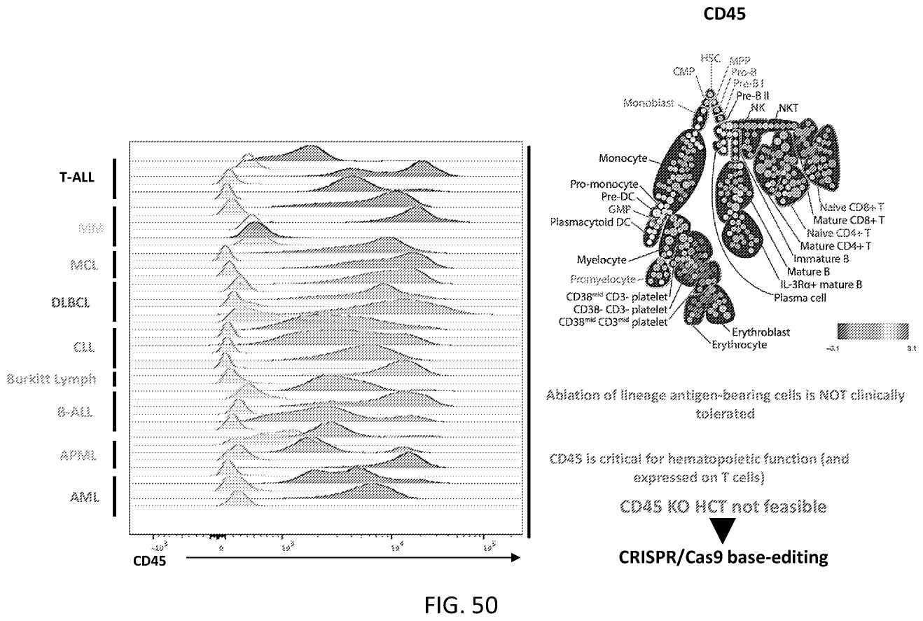

CD45 is the prototypical hematopoietic lineage antigen. A receptor tyrosine Phosphatase, CD45 is expressed on the surface of most nucleated hematopoietic cells during all phases of development, from immature stem cells to fully differentiated progeny (FIG. 2), with the major exception being mature erythrocytes. Interestingly, CD45 is not expressed on cells outside of the hematopoietic system. This fact of the biology of CD45 makes it a possible pan-hematologic antigen, the targeting of which with CAR T cells would effectively act as a single “drug” which could be used for all hematologic indications including cancers. Examples of such cancers include T-cell acute lymphoblastic leukemia (T-ALL), multiple-myeloma (MM), mantle cell lymphoma (MCL), diffuse large B-cell lymphoma (DLBCL), chronic lymphocytic leukemia (CLL), Burkitt's Lymphoma, B cell acute lymphoblastic leukemia (B-ALL), acute promyelocytic leukemia (APML), and acute myeloid leukemia (AML) among others. FIG. 50, left panel is a flow cytometry stain for CD45 expression in blood/Apheresis samples of patients with various blood cancers. All samples tested express CD45 with the exception with one multiple myeloma sample, which is consistent with previously published reports.

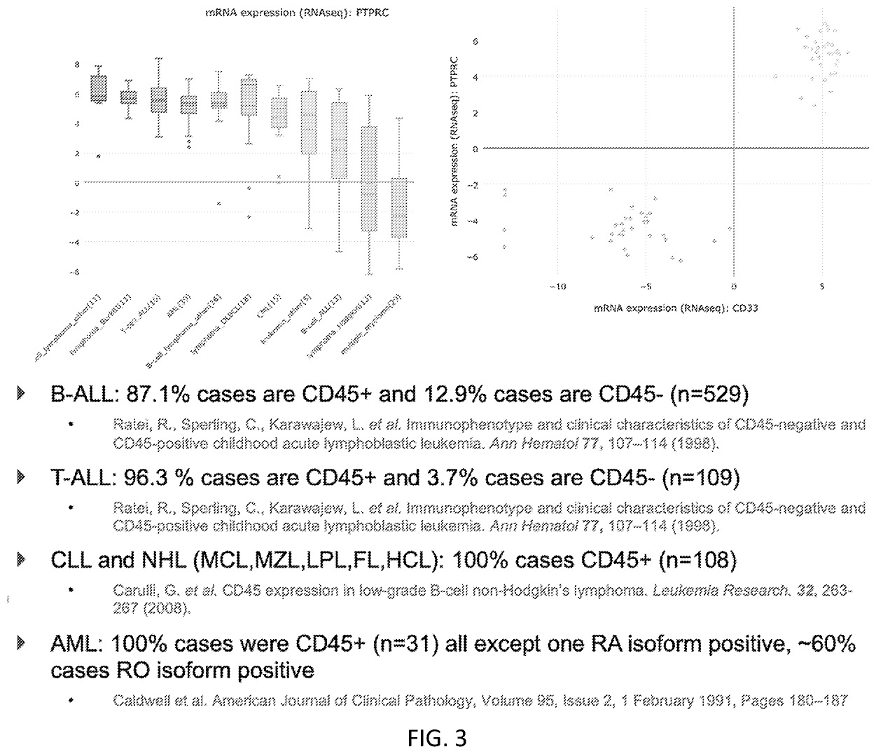

Indeed, as hematopoietic stem and progenitor cells (HSPC) undergo malignant transformation to AML, CD45 begins to co-localize to lipid rafts, contributing to increased GM-CSF signaling through regulation of the tyrosine protein kinase Lyn. CD45 inhibition impairs the motility and engraftment of immature human CD34+ cells and of AML blasts in immunodeficient mice and HSPC in CD45KO mice also exhibit defective motility and reduced homing related to impaired Src kinase activity (FIG. 3). Without wishing to be bound by theory, these observations raise the possibility that CD45 could serve as a previously unrecognized “Achilles Heel” antigen in AML, since leukemia clones that downregulate or lose CD45 expression would be functionally impaired. To this end, a radiolabeled anti-CD45 monoclonal antibody, Iomab-B, is currently being tested in a phase 3 trial as a conditioning regimen prior to allogeneic stem cell transplantation in patients with refractory AML.

One complication of targeting CD45 is that it is widely expressed by nucleated hematopoietic cells. FIG. 50, right panel is a CyTOF panel showing that all nucleated hematopoietic cells express CD45 at high levels (figure from Bendall, et al. Science (2011) 332(6030):687-96). Therefore, CD45 CAR therapy is predicted to have on target off tumor toxicities that cannot be tolerated. The studies of the present disclosure seek to circumvent these toxicities by editing the germline CD45 locus in human HSCs and combine an edited transplant with the CAR45 T cells to regenerate blood while all blood cancer cells remain targets of the CD45-specific CAR T cells.

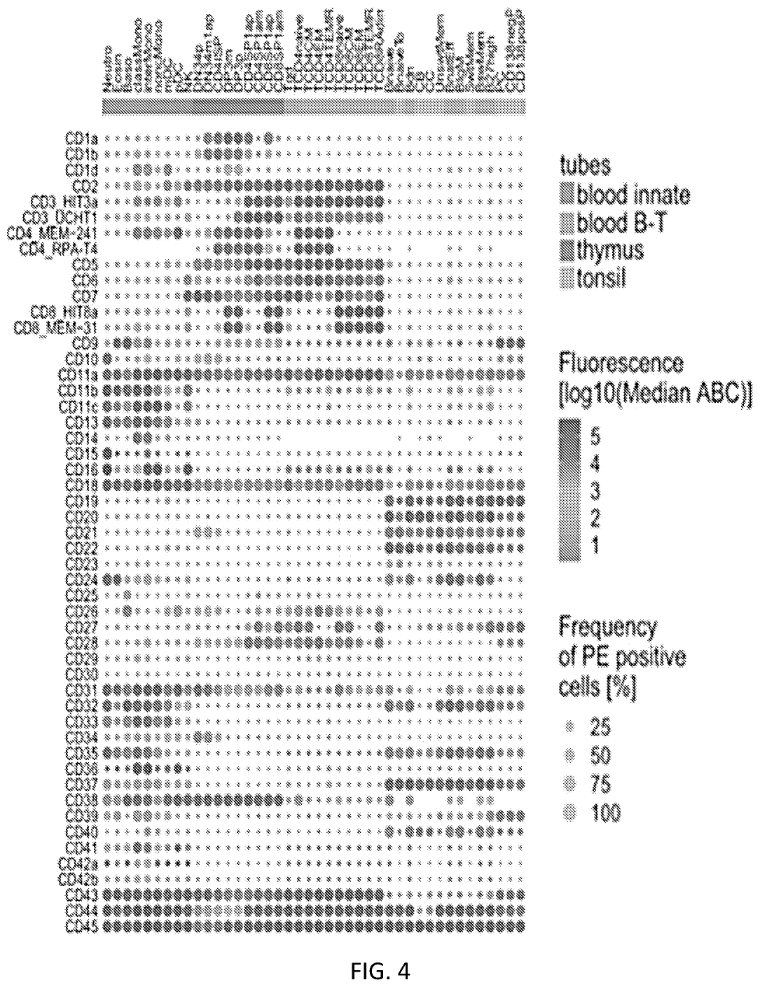

In addition to directly targeting hematopoietic cancers, CD45-directed CAR T cells could also be used to for “sterilizing” ablation of hemopoietic cells. Existing “conditioning” regimens that are used prior to bone marrow stem cell transplants use combinations of highly toxic chemotherapies and whole-body irradiation. Given the hematopoietic-specificity of CD45 expression (FIG. 4), CAR targeting would thus allow for a complete immune reset with a significantly reduced need for chemotherapy and/or radiation.

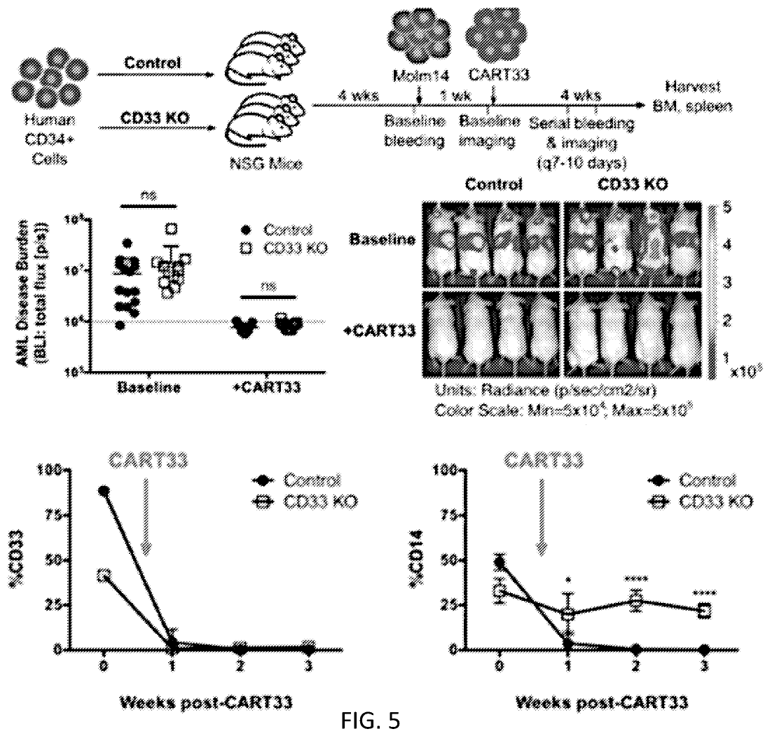

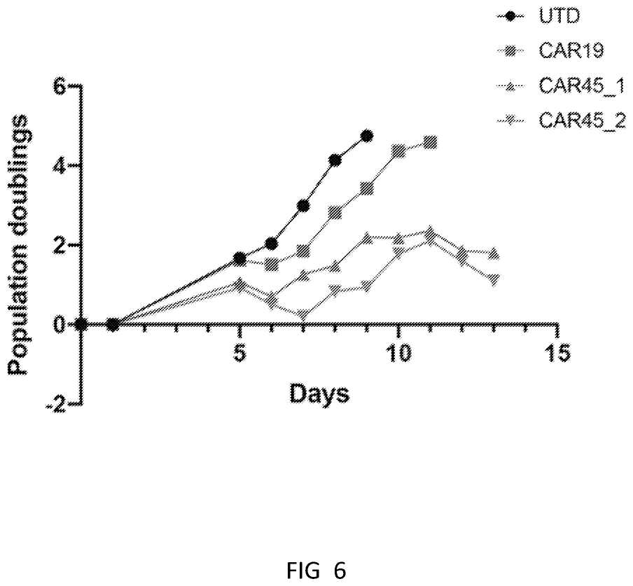

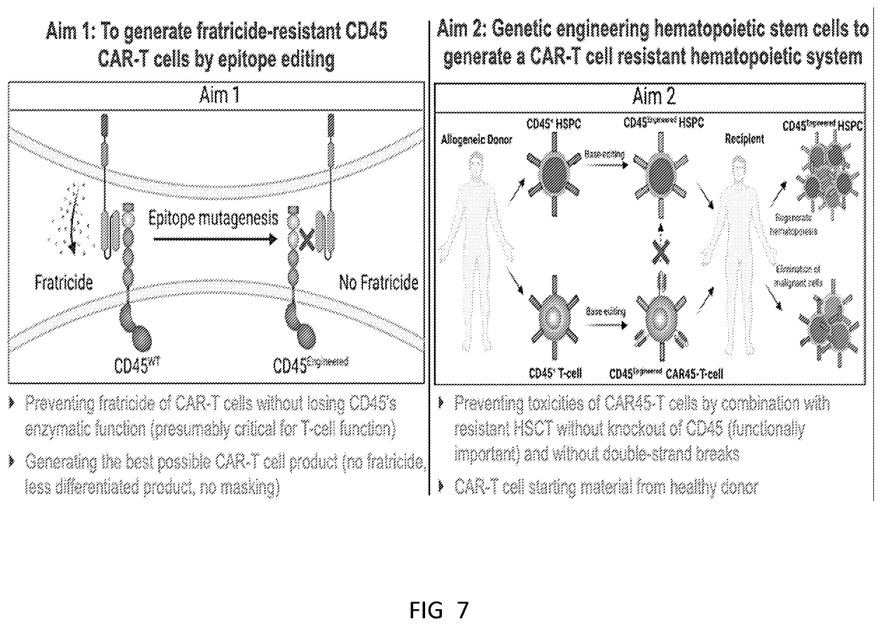

Administration of CAR45 T cells is expected to result in long term depletion of all hematopoietic cells. However, the bone marrow must be allowed to recover to prevent fatal toxicities. Previous studies using CD33-directed CAR T cells developed a novel approach, wherein CD33 deleted HSPC are combined with anti-CD33 CART cells as a tandem therapy to overcome toxicities from prolonged anti-CD33 immune pressure (FIG. 5). Here, the CD33 negative HSPCs re-engraft the patient while the CAR T cells deplete both tumor cells and endogenous bone marrow cells. This approach has certain limitations, however, in that certain hematopoietic cell populations must express CD33, and their long-term depletion is associated with unavoidable toxicities.

### Example 2: Development of a CD45-Specific CAR

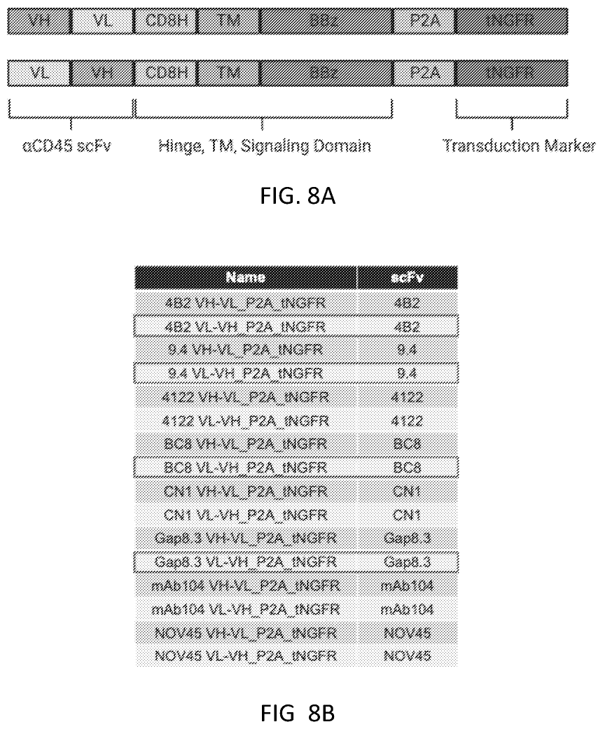

In order to create CD45-specific CAR constructs, studies started with heavy and light variable chains derived from eight publicly-available anti-CD45 antibodies: 4B2 (disclosed in U.S. Pat. No. 5,843,684), 9.4 (disclosed in WO2017/009473), BC8 (disclosed in U.S. patent application Ser. No. 16/298,202), CN1 (disclosed in Chinese patent application CN106046163), Gap8.3 (publically available through ATCC), mAb104 (disclosed in WO2020/092655), and NOV45 (disclosed in WO2005/026210). Variable chains from each antibody were assembled into VH/VL and VL/VH versions of each scFv (see FIG. 8B). All scFvs were then cloned into a commonly used CAR constructs consisting of a CD8 hinge and transmembrane domain, a 4-1BB costimulatory domains as well as a CD3 zeta signaling domain. The constructs also included tNGFR downstream of a 2 A ribosomal skip element that can be used to measure transduction and use as a surrogate marker for CAR positive cells (FIG. 8A).

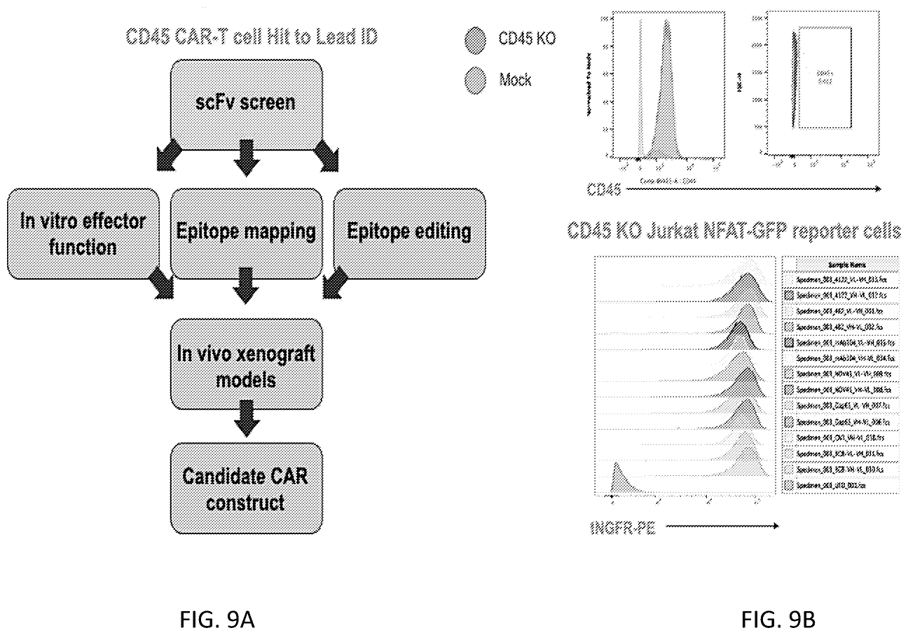

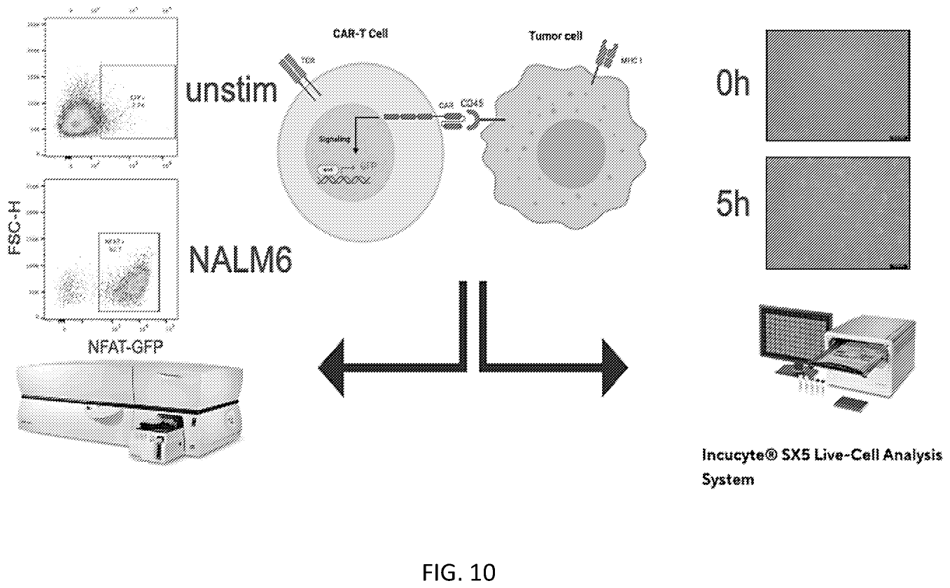

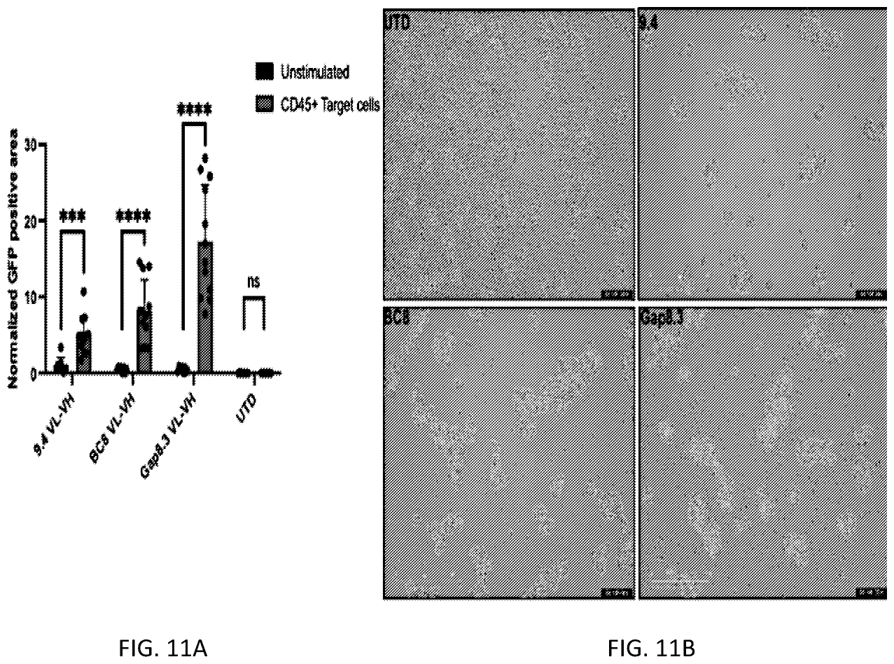

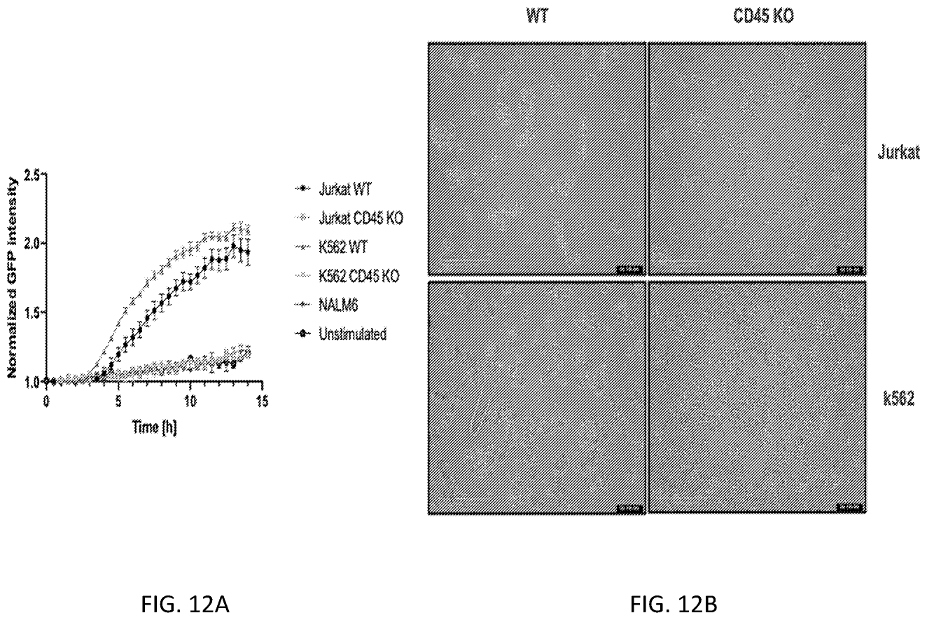

Jurkat cells expressing GFP under the nuclear factor of activated T cells (NFAT) minimal promoter were used to assess T cell activation in response to CAR-T cell stimulation when co-cultured with tumor cells expressing CD45. To prevent fratricide and tumor cell independent activation, CD45 was knocked out in the Jurkat cells using CRISPR/Cas9 and purified via MACS columns. The sgRNA used had the sequence 5′CCCCCATGAACGTTACCATT′3 (SEQ ID NO: 212) and targets exon 14 of the PTPRC gene (encoding for CD45). The CD45 knockout Jurkat NFAT-GFP reporter cells were subsequently transduced with the CAR45 constructs and expressing cells were sorted on tNGFR in order to purify CAR-expressing cells (see FIG. 9B). These reporter cells were then used in GFP expression experiments in which CAR activation leads to the expression of GFP, which is measured by flow cytometry and time-lapse fluorescent microscopy (see FIG. 10). From the NFAT-GFP reporter screen, multiple constructs were found to be significantly functional, including 9.4 VL/VH, BC8 VL/VH, and Gap8.3 VL/VH (FIGS. 11A and 11B). In order to demonstrate CAR specificity for CD45, CD45 expression was knocked-out in Jurkat and K562 cells using the sgRNA of SEQ ID NO: 212. These cells were the co-cultured with NFAT-GFP CAR expressing Jurkat cells. NALM6 cells were used as a negative control, as this cell line is CD45 negative. When co-cultured with the CD45 positive cell lines, the CD45-directed NFAT-GFP CAR Jurkat cells express GFP, but not when co-cultured with the same cell line that has undergone CD45 deletion (FIGS. 12A-12B).

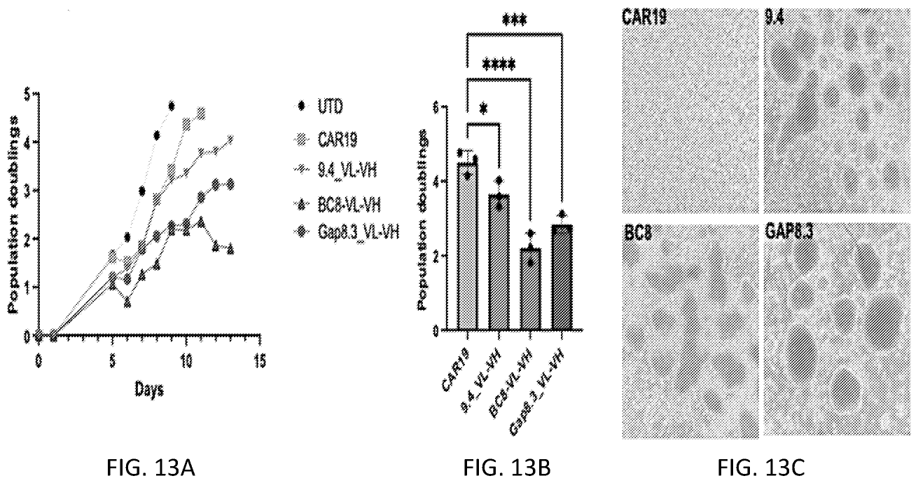

Evidence of fratricide was then assessed in primary human T cells. Here, human T cells were activated with CD3/CD28 dynabeads and lentivirally transduced with the respective CAR constructs. As expected, CAR45-T cells show evidence of fratricide in diminished numerical yield compared to CD19-specific CAR T cells (CAR19) (FIGS. 13A-13B). Microscopic observation of the T cell cultures also revealed that the CAR45 T cells cluster during ex vivo activation and culture as they engage with CD45 on neighboring T cells while CAR19 T cells do not (FIG. 13C).

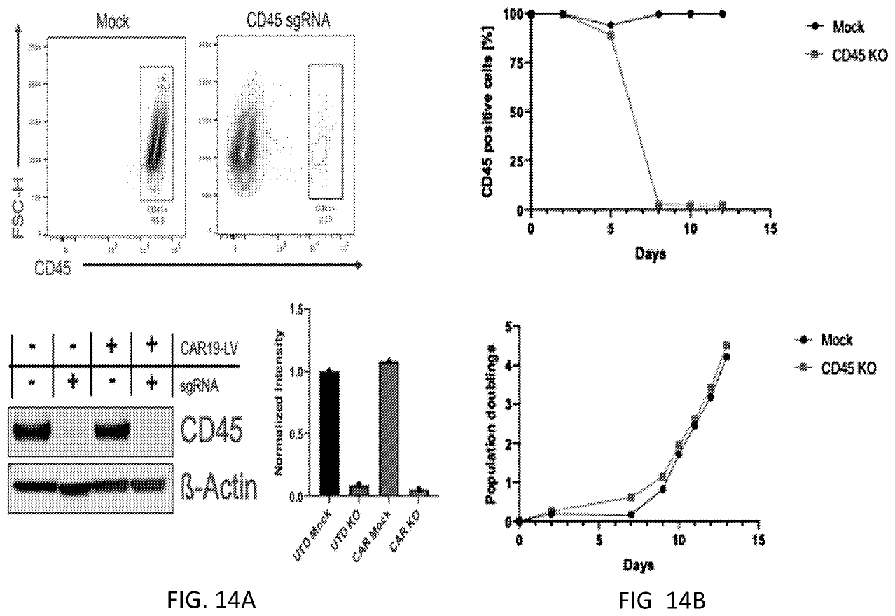

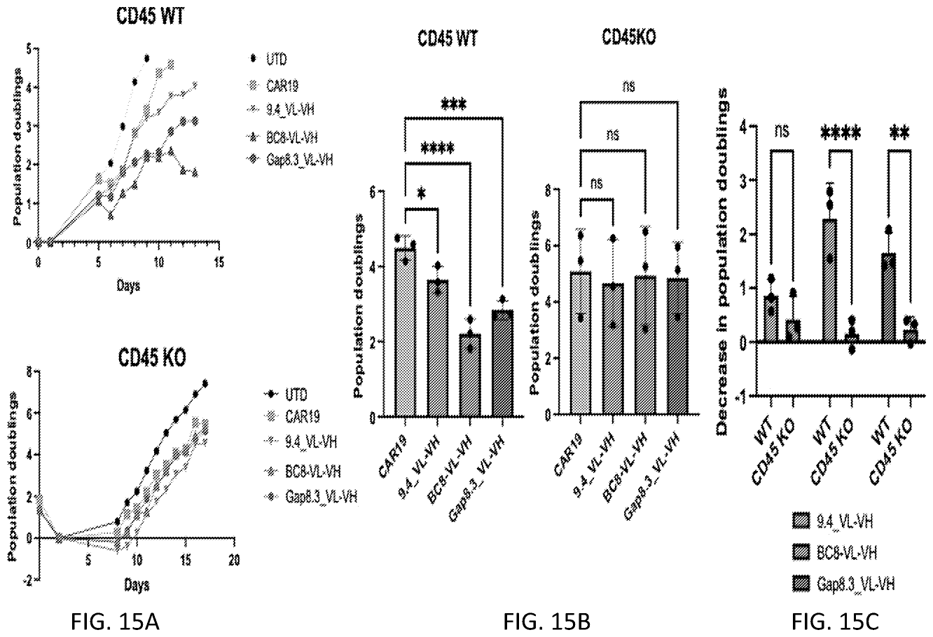

To prevent fratricide during ex vivo expansion, a CRISPR/Cas9 nuclease system was used to delete CD45 in primary human T cells prior to CAR transduction. Unstimulated T cells were electroporated with SpCas9 and an sgRNA targeting exon 14 (5′CCCCCATGAACGTTAC CATT′3 SEQ ID NO: 212). Cells were rested for 2 days at 30° C. and subsequently activated with CD3/CD28 dynabeads. After 5 days of bead stimulation, beads were removed and T cell expansion and CD45 expression was monitored (FIG. 14A). The sgRNA results in a very efficient knockout of CD45 in over 95% of the T cells, and this efficiency is sustained until the end of expansion. Results demonstrated that CD45KO and mock transfected control cells had similar levels of proliferation suggesting that CD45 expression is not required post T cell activation (FIG. 14B). Knockout was verified by flow cytometry, western blotting and tracking of indels by decomposition. Subsequent experiments reinforced the observation that deleting CD45 from the CAR-T cells can prevent fratricide of the CAR45 T cells, as these cells are capable of expanding similarly to CAR19 T cells. The benefits of CD45 ablation on proliferation was also observed for all three CD45 CAR constructs (FIGS. 15A-15C).

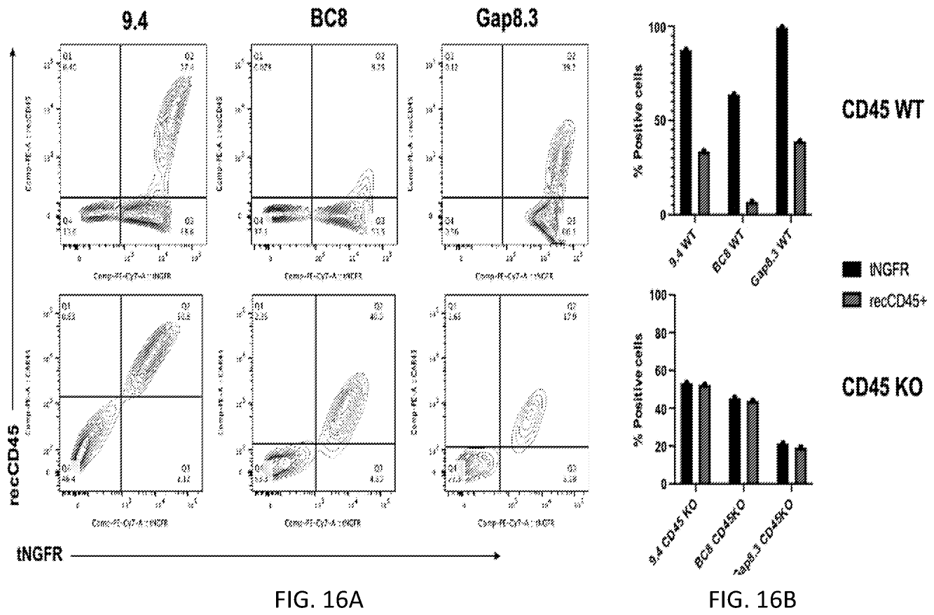

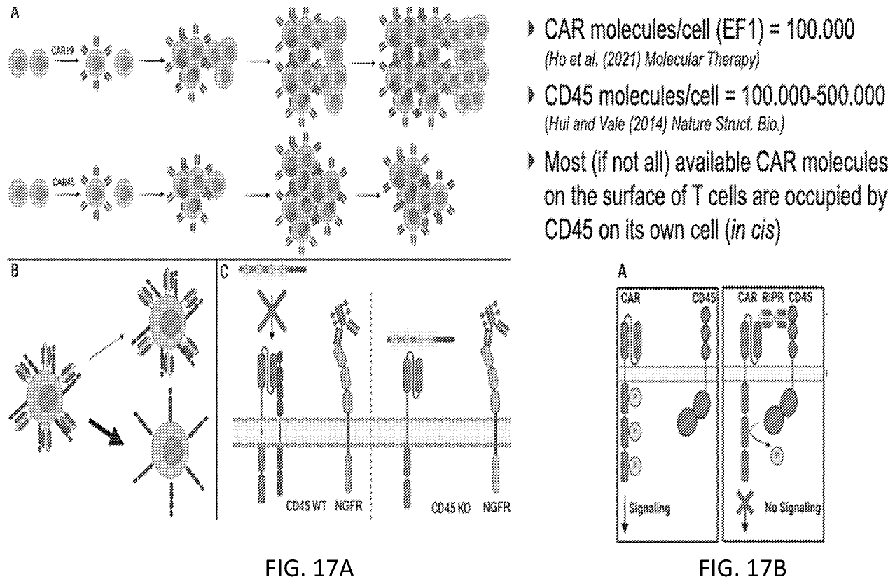

When comparing CD45 positive (WT) and CD45 deleted CAR45 T cell expansions, a self-enrichment of CAR+ T cells in the CD45 positive group was observed, suggesting that CAR45-T cells preferentially eliminate untransduced cells during ex vivo culture. Additionally, it was also observed that not all transduced cells (tNGFR+) were CAR positive (recCD45) (FIG. 16). One mechanism that could explain these observations is that CAR positive T cells can partially “protect” themselves from other CAR T cells by masking the epitope on their own cells (in cis). This mechanism is supported by the observation that not all transduced cells (tNGFR+) are CAR positive (as measured by staining with recombinant CD45). Another mechanistic explanation would be that expression of the CAR is downregulated during ex vivo expansion due to constant antigen exposure, though neither of these explanations are mutally exclusive. Either way, these phenotypes can be rescued by deleting CD45 expression during ex vivo expansion. An illustration of this potential phenomenon is found in FIG. 17.

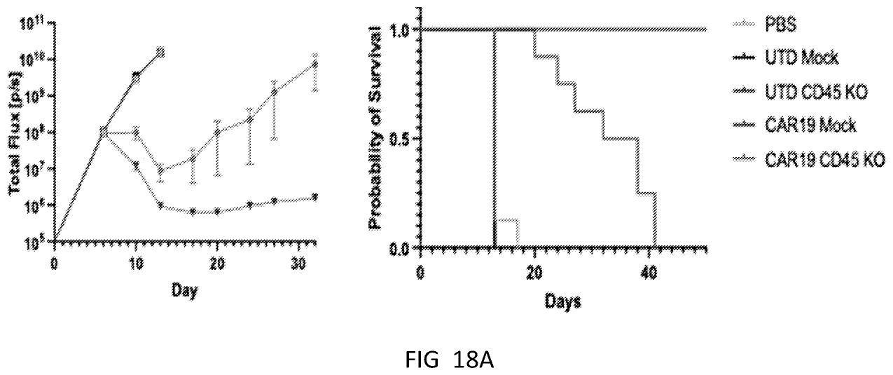

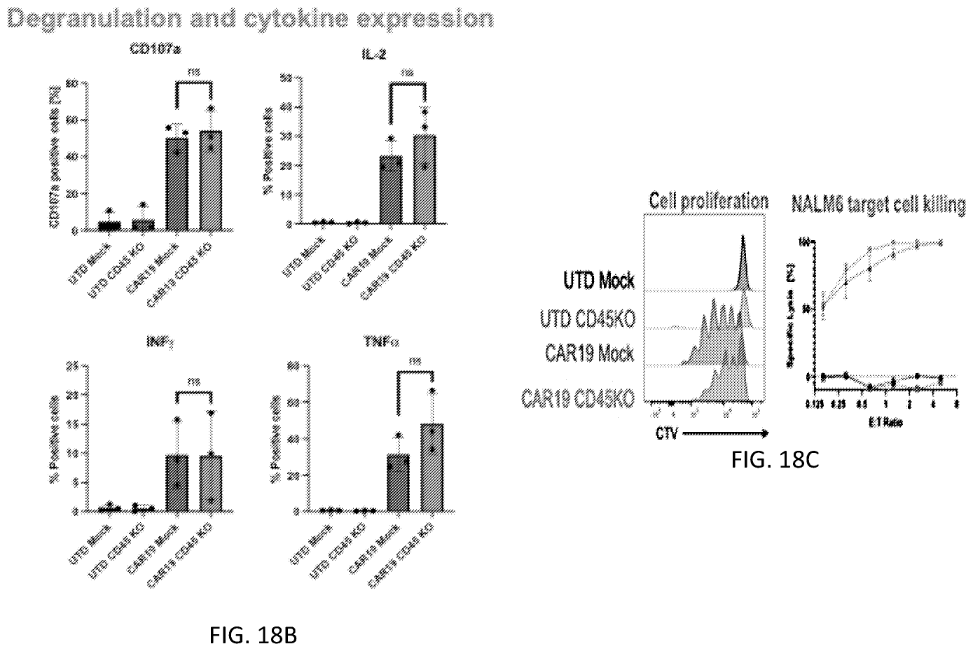

As described previously in this disclosure, CD45 deletion is one strategy to overcome fratricide and masking of CAR45 T cells. In order to investigate the functional effects of CD45 deletion, T cell function was assessed using the established CAR19-NALM6 tumor model in several effector assays in vitro. Here, T cells expressing a CD19-specific CAR are incubated with the CD19-expressing tumor cell line NALM6. Results demonstrated that CD45 deleted CAR19 T cells did not have any deficit in their ability to produce and secrete cytokines and kill tumor cells in vitro (FIG. 18B/C). However, they do have a mild but significant defect in proliferation in response to tumor cell co-culture (FIG. 18C). In vivo, this defect would likely result in incomplete tumor clearance followed by significant tumor outgrowth as compared to CD45 positive CAR19 T cells. Subsequent in vivo studies demonstrated this to be the case (FIG. 18A). Here, implantation of one million tumor cells was followed 7 days later by an injection of 3 million CAR-T cells. Although CD45 KO CAR-T cells are not as efficacious in vivo, they can be used as a tool to evaluate different CAR45 constructs without interference from tumor-cell independent activation/fratricide.

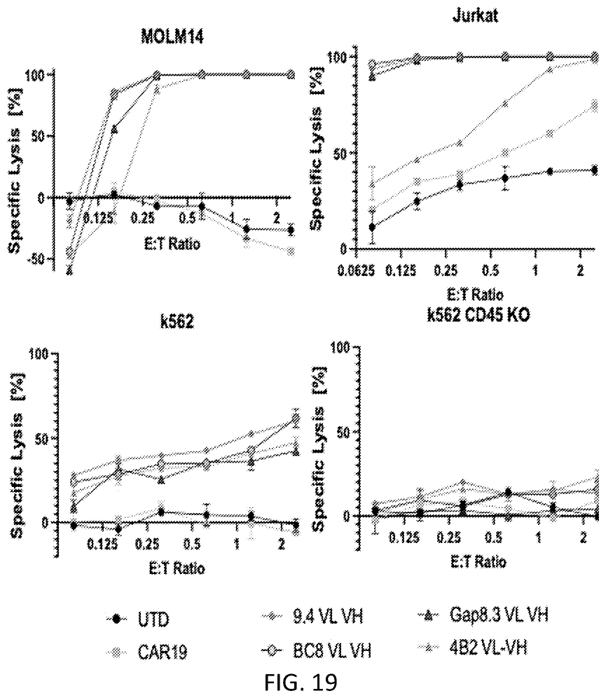

Studies were then conducted in order to test the ability of CAR45 T cells to lyse tumor cells in vitro. CAR45 T cells were co-cultured with various CD45+ tumor cell lines which had been modified to express luciferase, which enables the measurement of cell lysis by luminescence. The cell lines selected were MOLM14 (an AML line), Jurkat (a T-ALL line), k562 (a CML line), and k562 cells in which CD45 expression had been knocked out by CRISPR similar to previous studies disclosed herein. All 4 constructs tested were able to lyse the three CD45-expressing target tumor cells across a range of effector to target ratios (Effector=CAR-T cells, Target=Tumor cells) (see FIG. 19). Cytotoxicity was measured by bioluminescence 24 and 48 hrs of coculture. As expected, lysis of the CD45 knockout k562 cells by each CAR45 construct was minimal.

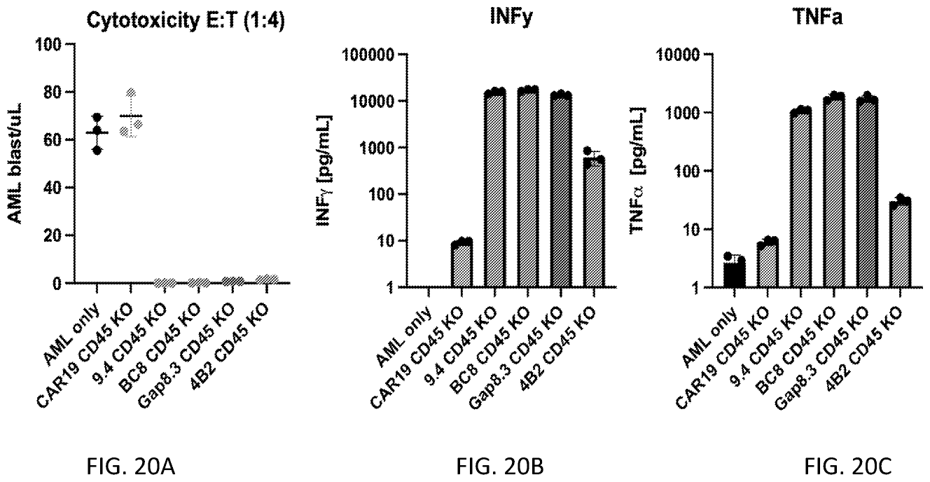

Similar in vitro lysis studies were then performed using AML cells derived from a human patient. Here T cells expressing each of the three CAR45 constructs were co-cultured with patient tumor cells and AML blasts were counted by flow cytometry (CD3−/CD33+/CD45+) after 24 and 48 hours of co-culture (FIG. 20). Results demonstrated that each construct as able to specifically lyse the patent-derived AML cells.

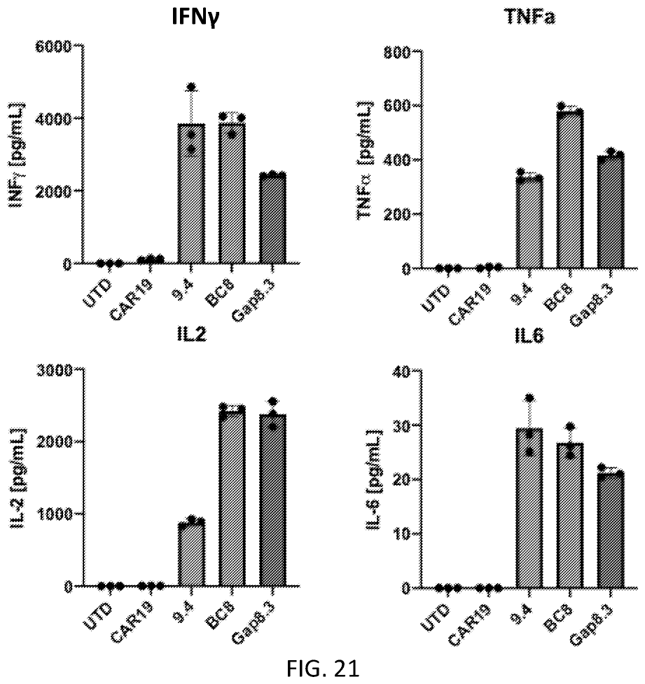

Next, the ability of CAR45 T cells to secrete effector cytokines when co-cultured with various target tumor cells in vitro was assessed. CAR45 T cells were co-cultured with MOLM14 tumor cells and the secretion of cytokines were measured by cytometric bead array (FIG. 21). T cells expressing each of the three CAR45 constructs tested expressed significant levels of IFNγ, TNFα, IL-2, and IL-6. CAR19 expressing T cells were used as a negative control, and as expected failed to secrete any of the tested cytokines.

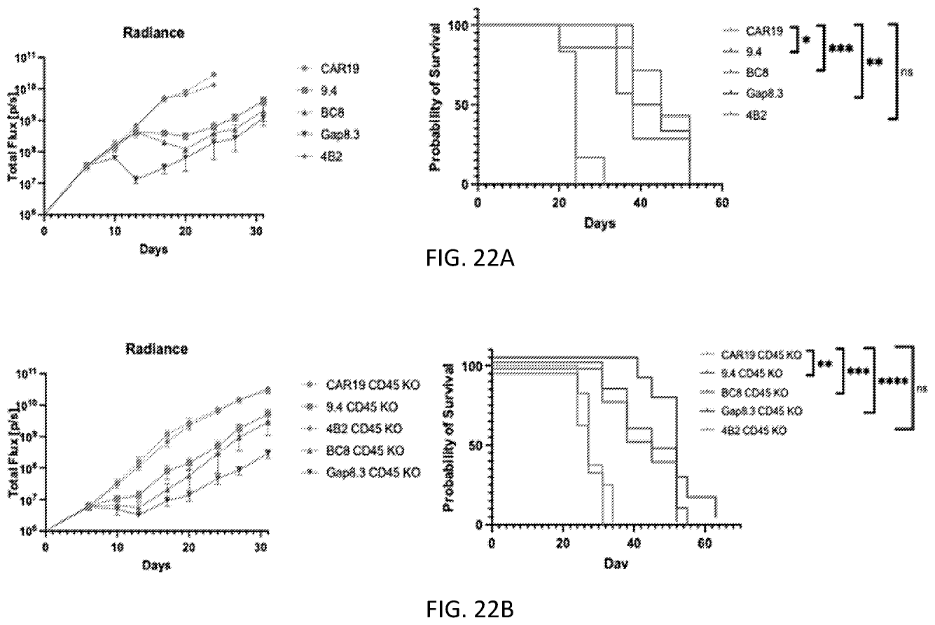

A series of in vivo mouse studies were then conducted. As expected CAR45 T cells are able to reduce tumor burden and extend survival in tumor xenograft mouse models. However, their full potential is limited by fratricide in the CD45 expressing CAR45-T cells (FIG. 22A) and by the lack of CD45's function in T cell signaling in the CD45 knockout CAR45-T cells (FIG. 22B).

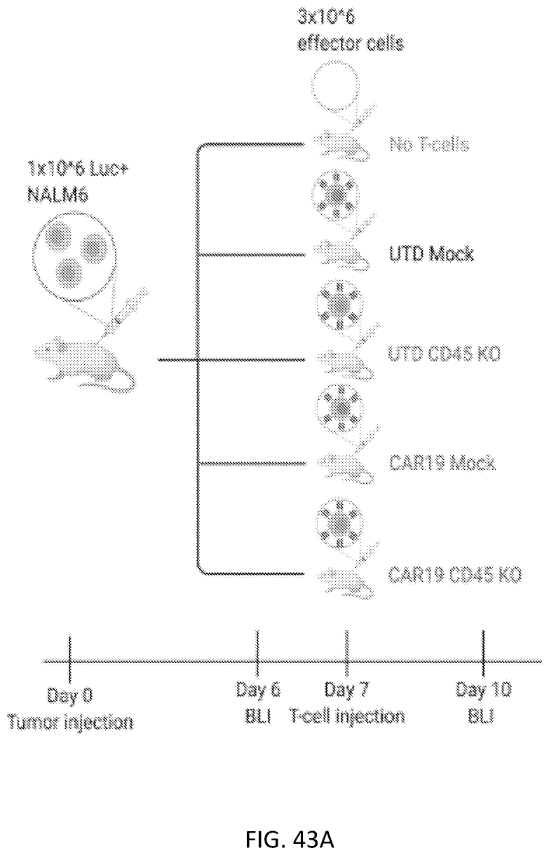

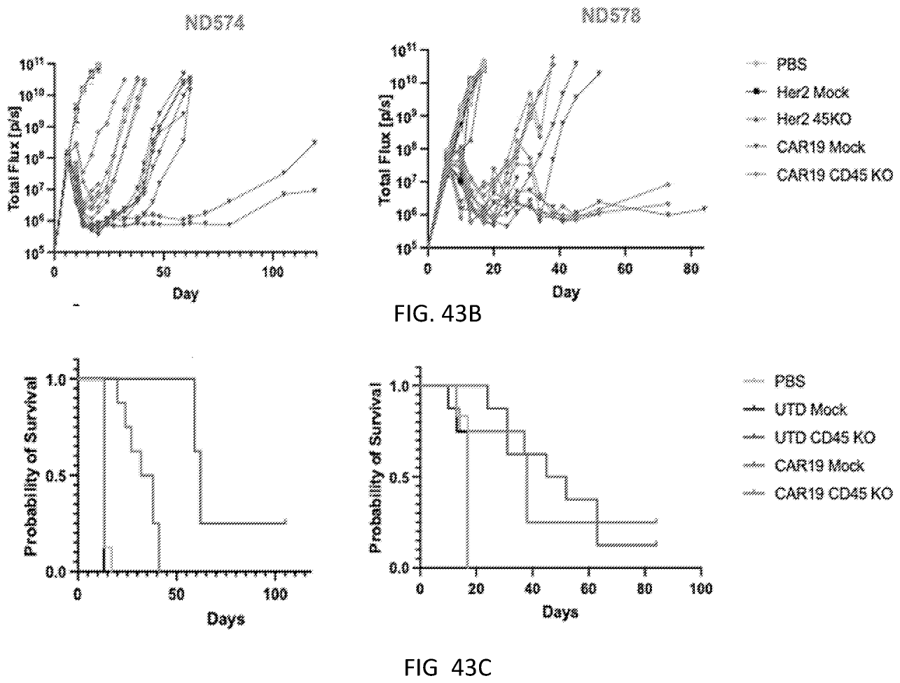

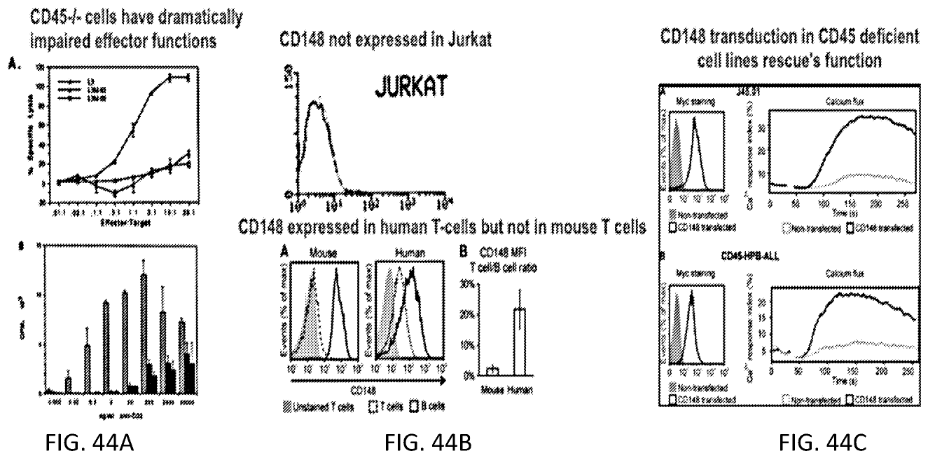

In order to determine whether the negative effects of CD45 ablation in CAR T cells also applied to other non-CD45 specific CARs, a series of studies was conducted using the anti-CD19 CAR (CAR19) in which CD45 was knocked out in human T cells transfected with the CAR19 construct. Similar to experiments using the CAR45, CAR19 CD45KO T cells demonstrated reduced proliferation in vitro as compared to CD45+ CAR19 cells (see FIG. 43A-43B). Next, a series of in vivo experiments was conducted in which mice bearing NALM6 xenograft tumors were injected with CAR19 CD45KO T cells. CAR19 alone, CD45KO alone, untouched T cells, and vehicle alone groups were used as controls (see FIG. 44A). Tumor growth (FIG. 44B) and survival (FIG. 44C) data from these studies demonstrated that, while CD45KO CAR19 cells were capable of initial anti-tumor efficacy, tumor outgrowth was faster and survival shorter than mice receiving CD45+ CAR19 T cells. Together these data demonstrate that CD45 plays a key role in maintaining the function of CAR expressing T cells, and that ablating CD45 expression in order to solve the problem of CAR45 fratricide does not result in restoring optimal cytotoxic function.

### Example 3: CD45 Modification to Avoid CAR T Cell Fratricide

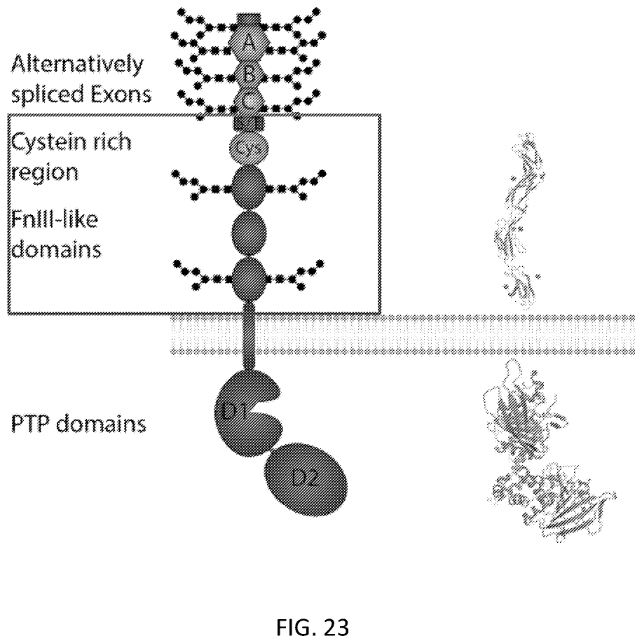

While CAR45 T cells lacking CD45 are functional in vitro and demonstrate potent anti-tumor efficacy, their in vivo tumor efficacy is likely impaired by the lack of CD45 as demonstrated by CAR19 CD45 KO experiments. To address this challenge, a series of studies were then undertaken to determine and edit the epitope of CD45 that is recognized by the CAR45 T cells in such way that CD45 is no longer recognized by the CAR, but the enzymatic function of CD45 remains sufficiently intact. Structurally, human CD45 is a relatively large glycoprotein (type I receptor) that consists of a large and rigid extracellular domain (heavily glycosylated) as well as a cytoplasmic tyrosine phosphatase domain (see FIG. 23). The extracellular domain contains 3 exons (Exon 4,5 and 6) with variable splicing patterns that give rise to different isoforms of CD45 (e.g. CD45RO which lacks all 3 alternatively spliced exons or CD45RA which has exon A(4) incorporated). Previous studies of the antibodies used to generate the CAR constructs of the current invention demonstrated that each was able to recognize all human CD45 isoforms, suggesting that their epitopes must lie within the constant domains of the CD45 extracellular domain (3 FN like domain repeats, one cysteine-rich region and a mucin like domain (very Nterminal). Crystal structures for the extracellular domains suggest a rigid “beads on a string” like conformation of the extracellular domain with the different sub-domains being stacked on top of each other in a rigid conformation.

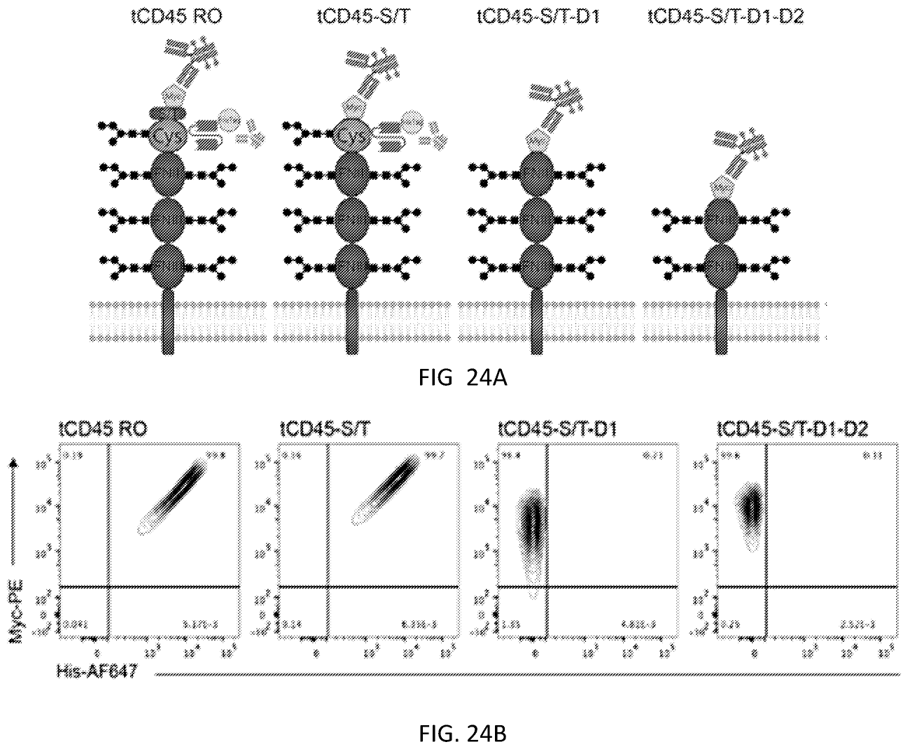

With the information obtained from the crystal structure (and given that FN domains are evolutionary conserved and stable structures), and without wishing to be bound by theory, it was hypothesized that the subdomains which are recognized by the CAR45-T cells can be narrowed down by sequentially truncating the extracellular domain. The constructs to generate the truncated CD45 constructs were de novo synthesized (FIG. 24A). To verify surface expression of the truncated CD45 constructs, all constructs were myc-tagged at the N-terminus to allow surface staining against the truncated CD45 expressing cell using anti-Myc antibodies followed by readout via flowcytometry. Cells expressing the truncated CD45 constructs were stained with recombinantly expressed, his-tagged scFv/antibody clones from which the CARs were made off. Positive staining for the his tag therefore only occurs when the scFv/antibody clones can bind truncated CD45. In FIG. 24B a recombinantly expressed scFv from the BC8 clone demonstrates that the D1 domain (Cys rich region) is required for binding of this clone, suggesting that the epitope lies within this domain.

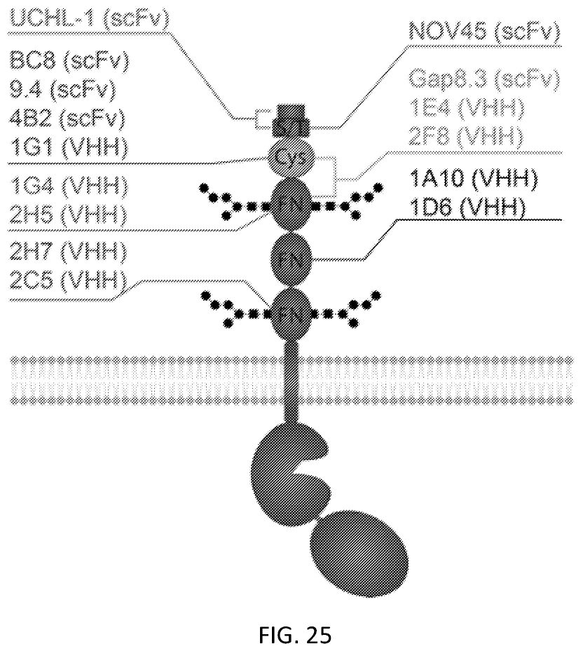

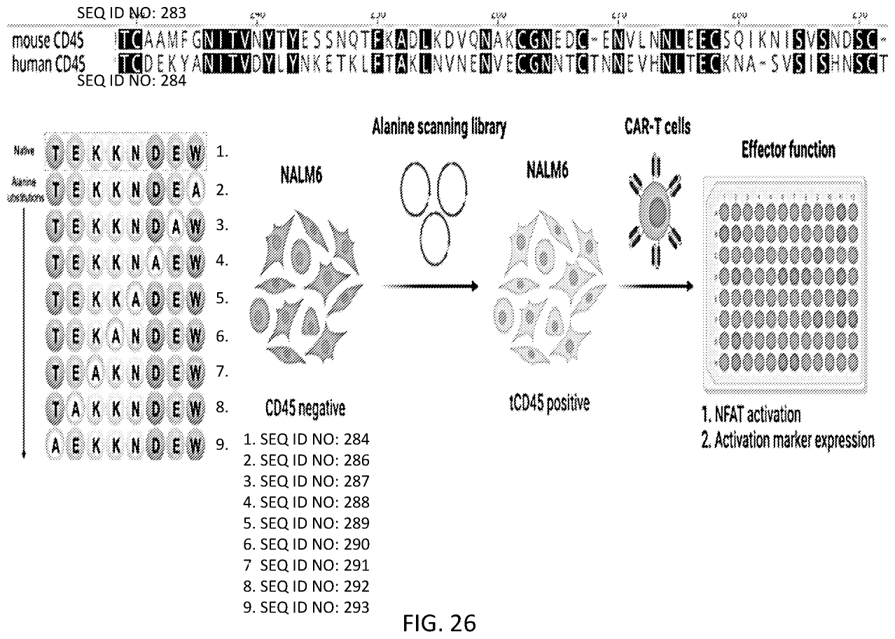

Similar studies with the truncated versions of CD45 were carried out with the other scFv's of the current invention. The diagram in FIG. 25 depicts where all scFv's/VHHs bind on the CD45 protein, including binding sites determined in previously published studies. Of note is the observation that most scFv's recognize the Cysteine rich region in part (Gap8.3) or in full (4B2,9.4 and BC8). While subdomain mapping drastically reduces the number of possible amino acids involved in the CAR-CD45 interaction, it does not identify the energetic “hotspot” amino acids that are critical for CAR recognition. Also, deleting an entire domain likely interferes with the proteins function and size-based exclusion from the immune synapse. Therefore follow-up studies revealing the amino-acid level epitope mapping were needed in order to edit only the amino acids relevant for CAR recognition. Since most relevant scFv's recognize the cysteine rich domain, mapping efforts focused on the CARs which recognize this domain. In order to identify these amino acids, an alanine mutagenesis scanning library was generated which covers the cysteine rich domain of CD45. Since all scFv clones recognize human, but not mouse CD45, it was hypothesized that the amino acids required for CAR recognition, at least in part, must be human specific. Therefore only substituted amino acids that are not conserved between mouse and human CD45 were covered in the library. This approach not only reduces the library size, but also decreases the likelihood that altering this amino acid by gene-editing in later applications results in a non-functional protein, as amino acids essential for maintaining function would likely be conserved between mice and humans. See FIG. 26.

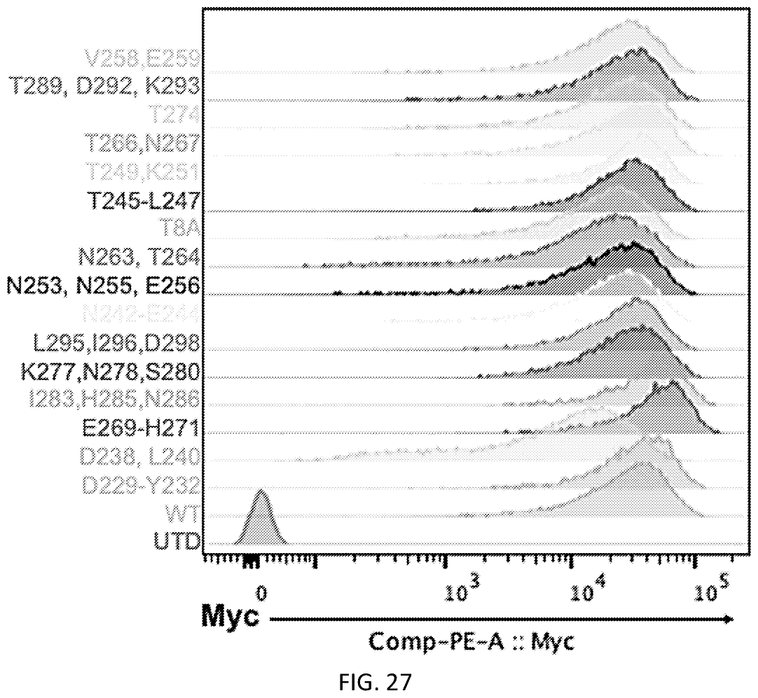

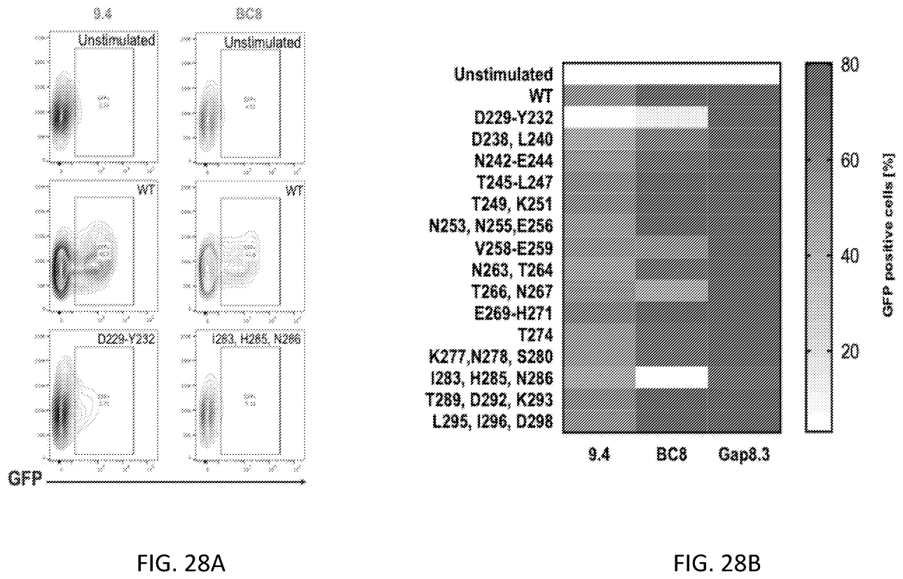

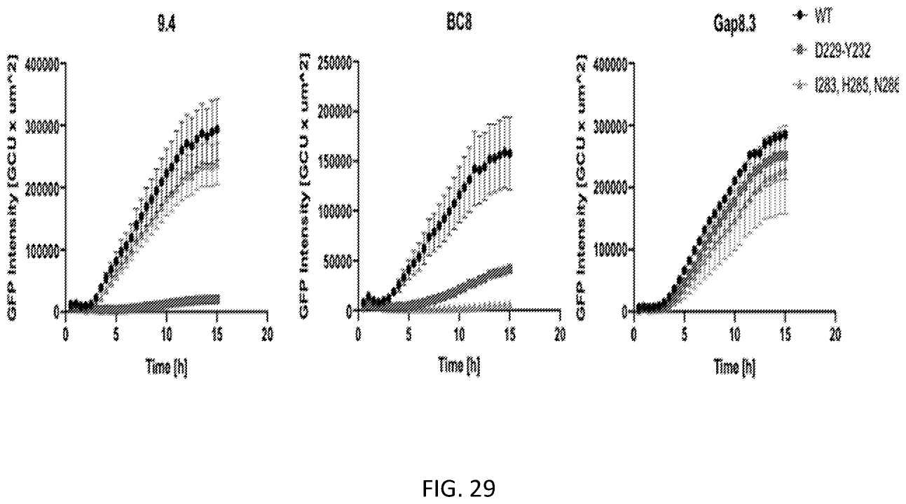

As a result, a library was generated that contained 1-4 alanine substitutions per construct. The alanine-mutated CD45 constructs were packaged in lentivirus and transduced into CD45 negative NALM6 cells. The alanine-mutated CD45 constructs all contained an N-terminal myc tag in order to verify expression and ensure the construct was transported to the cell surface. Staining of transfected cells with anti-Myc antibodies revealed robust expression of each mutant (FIG. 27). The transfected NALM6 were then co-cultured with different CAR45-NFAT-GFP Jurkat cells used in the studies previously described in the present disclosure and measured GFP expression as a readout for CAR activation (FIG. 28A). While each of the different CAR45 constructs recognized WT CD45 (as expected), they did not recognize certain alanine-mutated CD45 expressing cells. For example, clone 9.4 requires D229,E230,K231 amd Y232 for proper activation since co-culture with cells expressing alanine mutated D229-Y232 did not lead to proper CAR activation (FIG. 28B). For BC8, I283, H285 and N286 were required as well as a few others (T266,N267). This mutagenesis did not identify critical amino acids for Gap8.3. FIG. 29 shows a similar experiment using time lapse microscopy as a readout instead of flow cytometry.

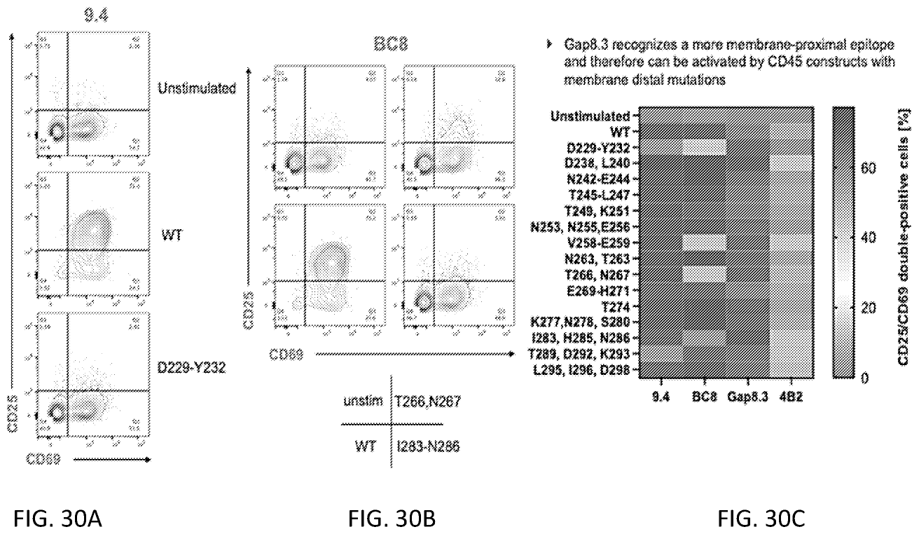

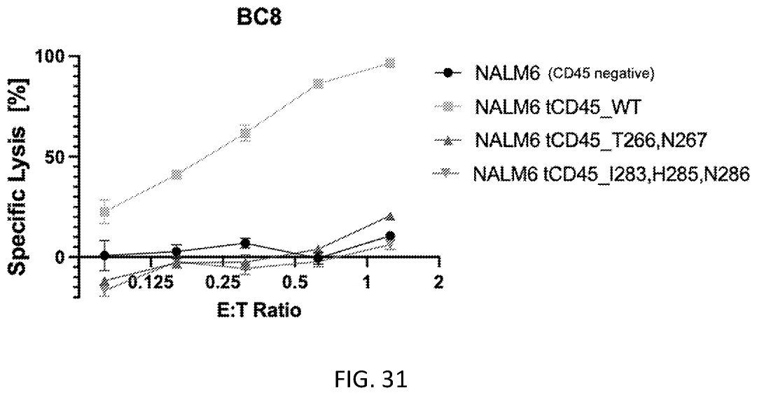

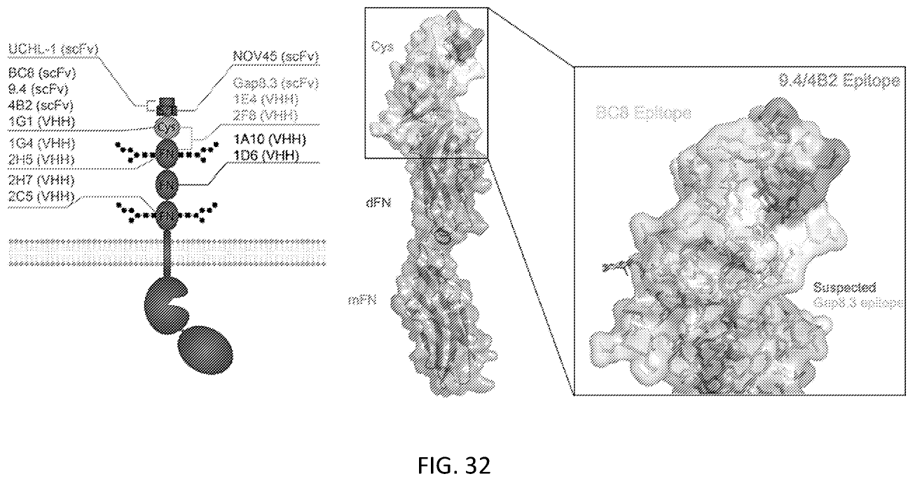

One caveat to the use of Jurkat reporter cells is that the activation threshold for Jurkat cells differs from that of primary human T cells. In order to validate the findings of the previous study more accurately, primary human CAR45-T cells were co-cultured with alanine-mutant NALM6 cells and expression of activation markers (CD25/CD69) was used as a readout for CAR T cell activation (FIG. 30A-30B). Results of these studies demonstrated the similarity between primary human T cells and Jurkat reporter cells and suggested that primary T cells are much more sensitive to perturbation of antigen recognition. The results of these studies allowed the identification of amino acids that are involved in CAR45-antigen interaction, but whose substitutions to alanine do not completely abolish CAR recognition (an example is V258-E259 or T266, N267 for BC8 or T289,D292,K292 for 9.4 (FIG. 30C). Follow-up cytotoxicity assays confirmed that NALM6 tumor cells expressing CD45 alanine mutants which fail to activate the CAR45-T cells are also not killed by CAR45 T cells, while CD45 WT transduced NALM6 are killed efficiently (FIG. 31). The results of these studies, which revealed the precise epitopes of each CD45-specific scFv used in the current invention are summarized in FIG. 32. Also illustrated is the crystal structure of the CD45 extracellular domain with the critical amino acids for each CAR superimposed.

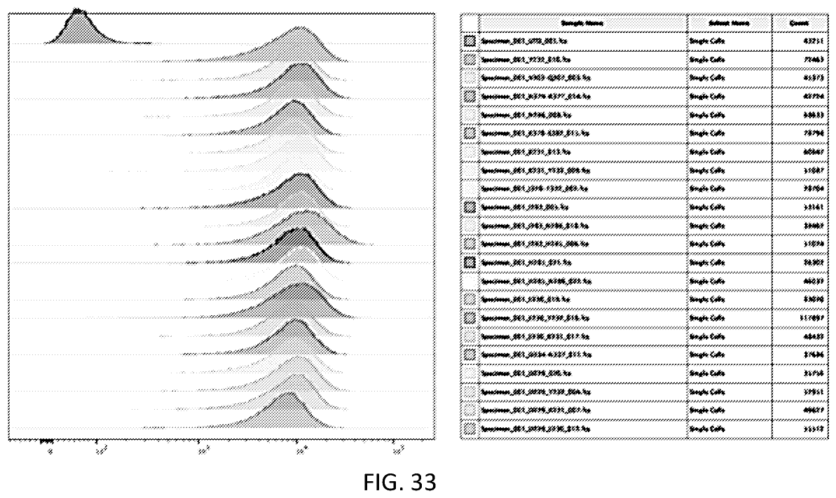

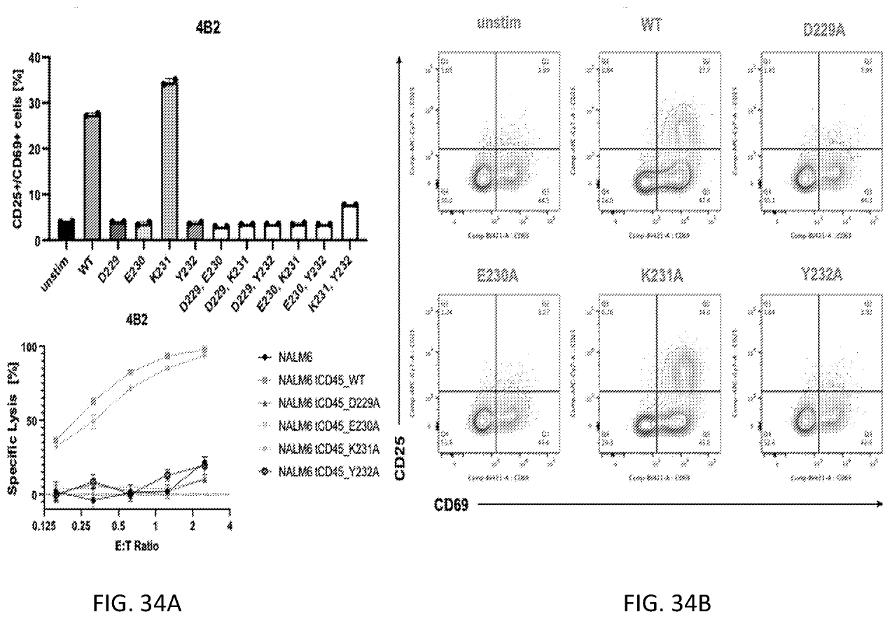

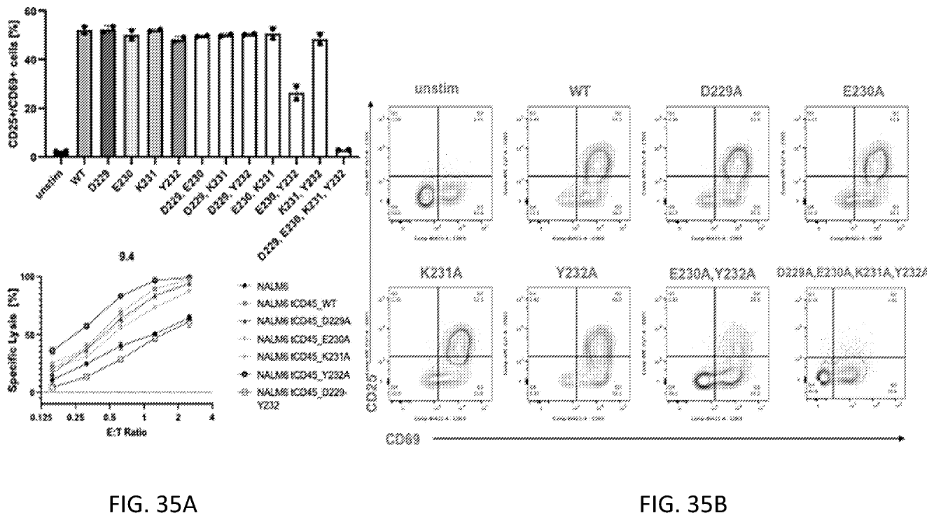

In the first alanine mutagenesis screen, most constructs had 2 or more alanine substitutions. To find the minimal essential sequence required for CAR45 recognition, a series of single alanine mutants identified in the previous studies was generated and expressed in NALM6 cells as previously described. FIG. 33 is histogram verifying expression of these single-alanine mutants as assessed by flow cytometry. Subsequence co-culture studies showed that the 4B2 CAR is very sensitive to single alanine mutations in the D229-Y232 region. Indeed, single mutants (except K231, FIG. 34A) were sufficient to completely abrogate CAR recognition and killing. (FIGS. 34A-34B). However, these studies also showed that the 9.4 CAR is less sensitive to single alanine mutations in the D229-Y232 region. Quadruple alanine mutants were required to abrogate CAR recognition (FIGS. 35A-35B). Importantly, this does not exclude the possibility that single mutants are sufficient to prevent CAR recognition when the mutation is not to alanine.

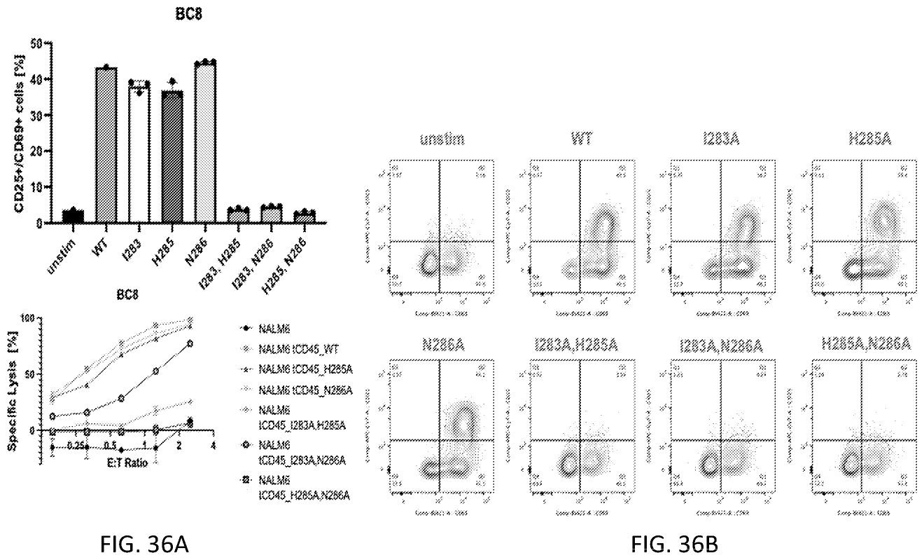

These studies also demonstrated that the BC8 CAR is sensitive to alanine mutations in the H285, N286 region (FIG. 36A). Single mutants were not sufficient to completely abrogate CAR recognition and killing but double mutants are (FIG. 36A-36B). Again, this does not exclude the possibility that single mutants are sufficient to prevent CAR recognition when the mutation is not to alanine.

### Example 4: Development of a CD45 Gene Editing System

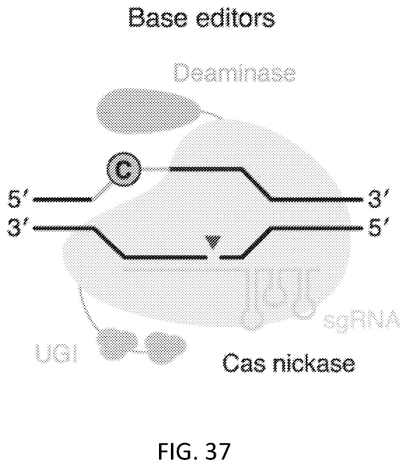

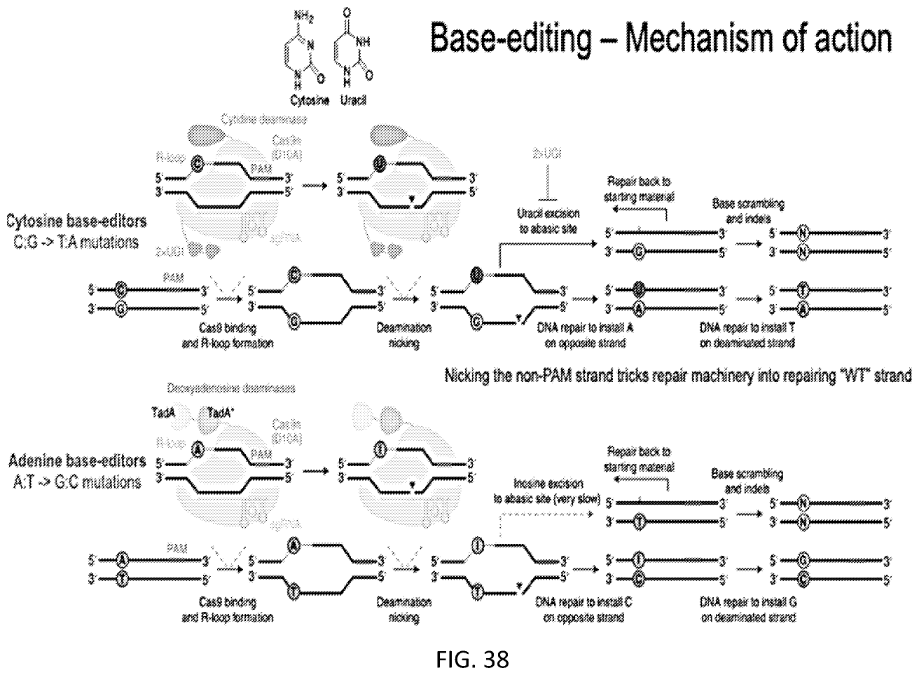

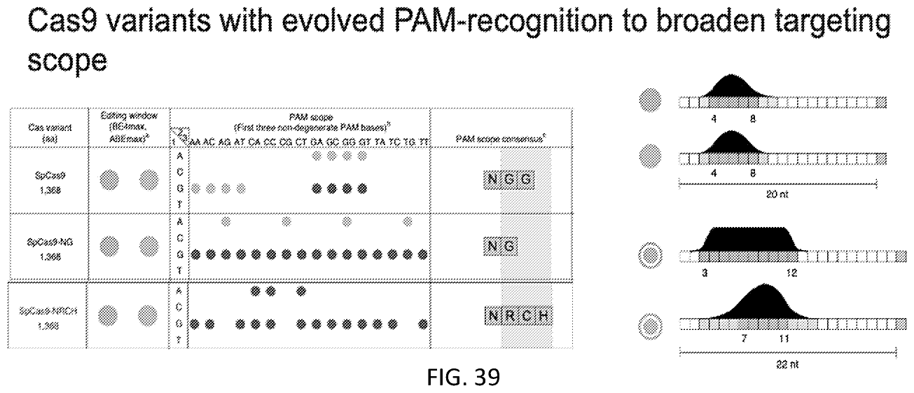

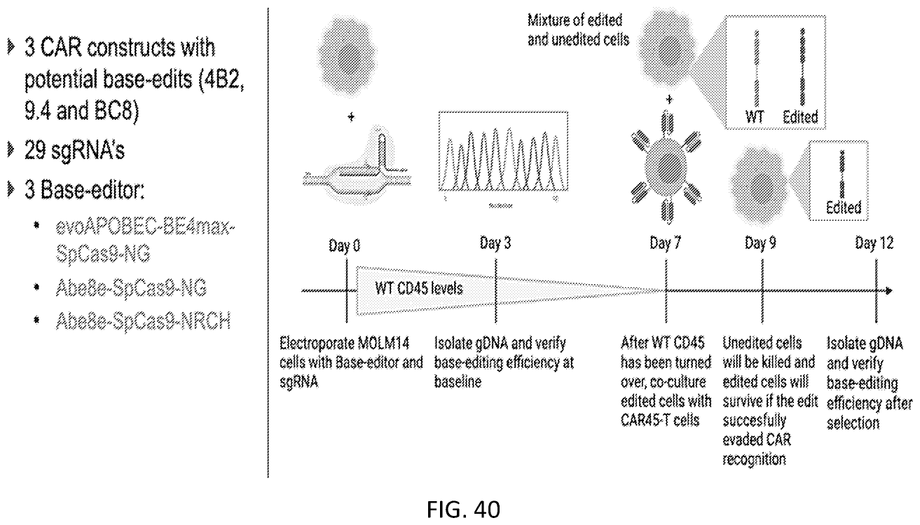

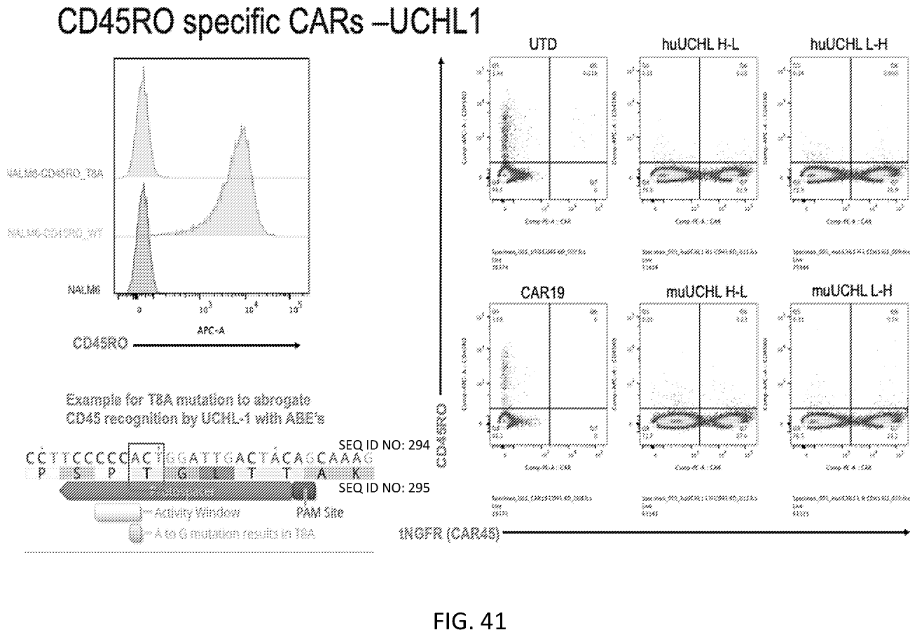

A key observation made by the alanine-substitution studies is that a series of non-synonymous mutations are needed to completely abrogate CD45 epitope recognition. For this reason, a CRISPR-Cas9-based base-editing system was adapted for use on CD45 so that several specific mutations to CD45 could be made simultaneously in the same reaction (see diagram in FIG. 37). One feature of these systems is that the Cas component of the base-editor has been modified to be less NGG-PAM restricted (See FIG. 39). While the list of possible base-editors is quite large, the studies of the current disclosure focused on adenine and cytidine base-editors that recognize either NG or NRCH PAMs. A diagram of the mechanism by which these editors perform their function is illustrated in FIG. 38. Subsequently, a base-editing screening strategy was developed that can abrogate the CAR45 epitopes on endogenous CD45. As part of this approach, 29 sgRNAs and 3 base-editors were developed to modify CD45 corresponding to each of the three CAR45 constructs (see FIG. 40).

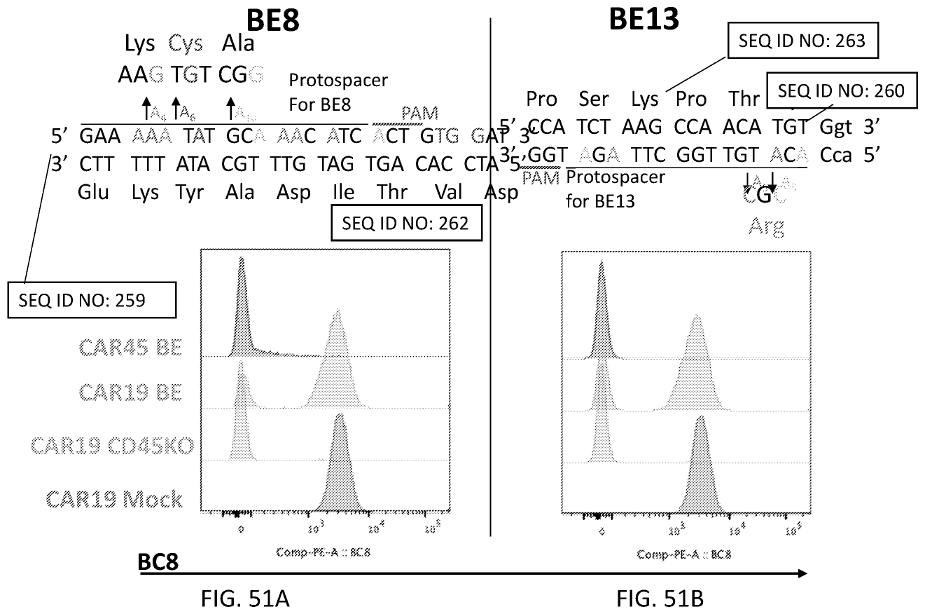

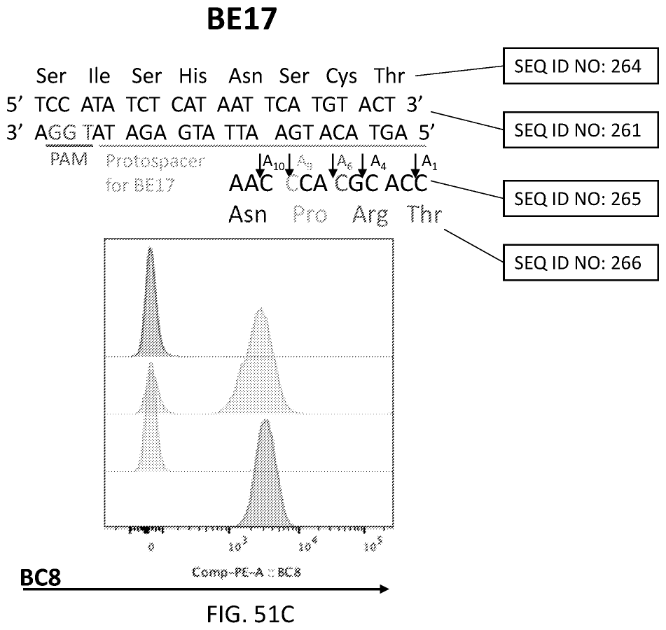

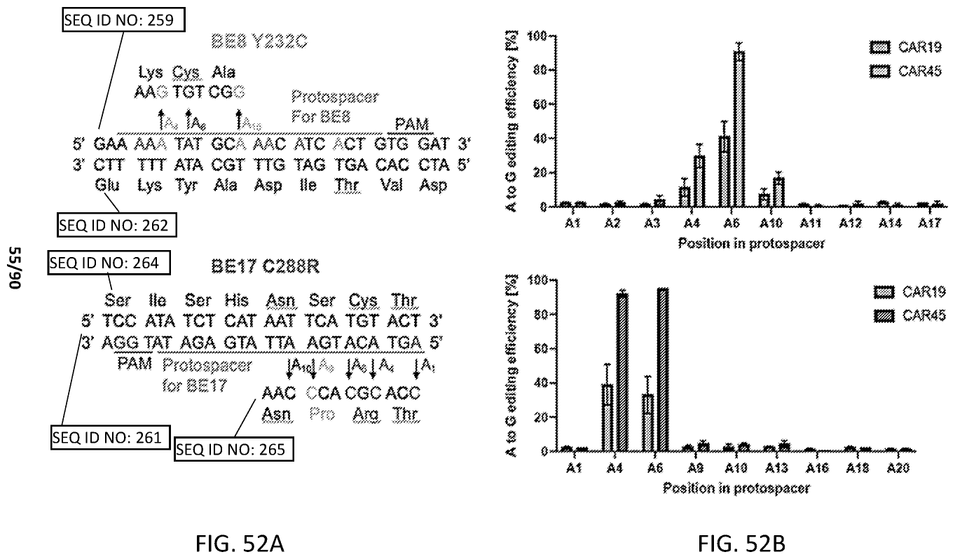

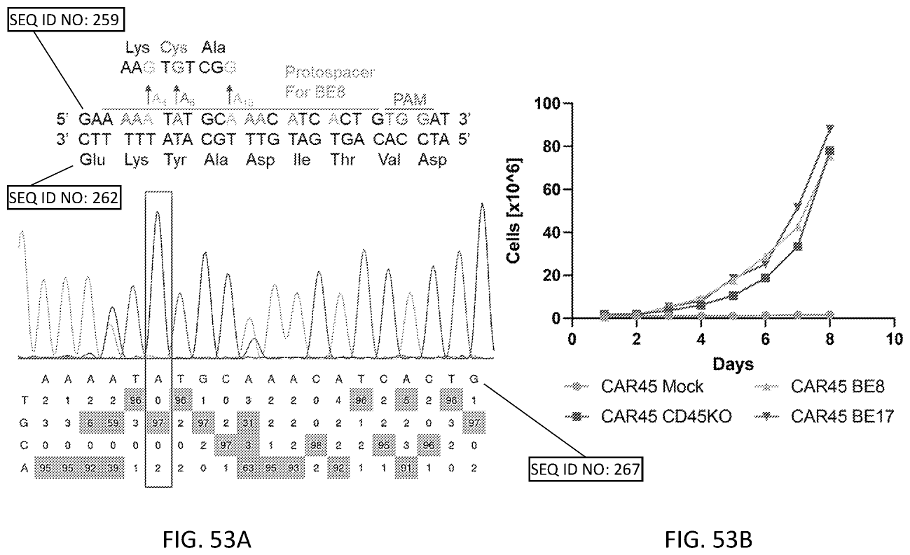

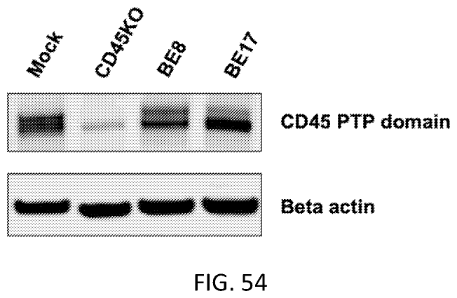

Of these, three were selected for further study: BE8, BE13, and BE17 (see FIGS. 51A-51C). The lower panel of each figure illustrates T cells that have been edited and stained with an anti-CD45 antibody (BC8) from which the lead CAR was made from. The three different base edits abolish binding of BC8 indistinguishable from CD45 knockout T cells. Without wishing to be bound by theory, these findings suggested that the antibody no longer binds to the edited CD45. Moreover, when T cells were transduced with the BC8 CAR45, enrichment occurs so that all T cells at the end of expansion are edited. Subsequent studies demonstrated that amplicon sequencing of the targeted locus showed enrichment of edits when T cells were transduced with CAR45 compared to CAR19 demonstrating protection and selection of edited alleles over time FIGS. 52A-52B. Sequencing of the edited loci revealed highly efficient editing at the target nucleotide (97%) for BE8. (FIG. 53A). Additionally, CD45 epitope base editing allowed for the expansion of CAR45 T cells to the same extent as CD45 knockout cells suggesting that fratricide has been prevented (FIG. 53B). Further, western blotting of protein lysates harvested from edited cells revealed that base-editing, unlike CD45 knockout using Cas9 nucleases, does not result in a loss of protein expression (FIG. 54).

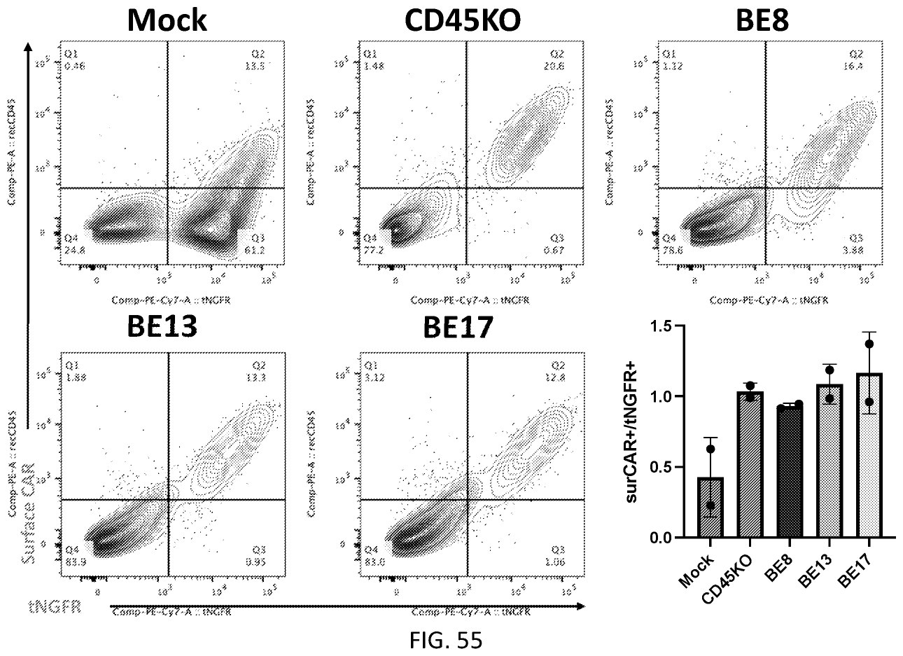

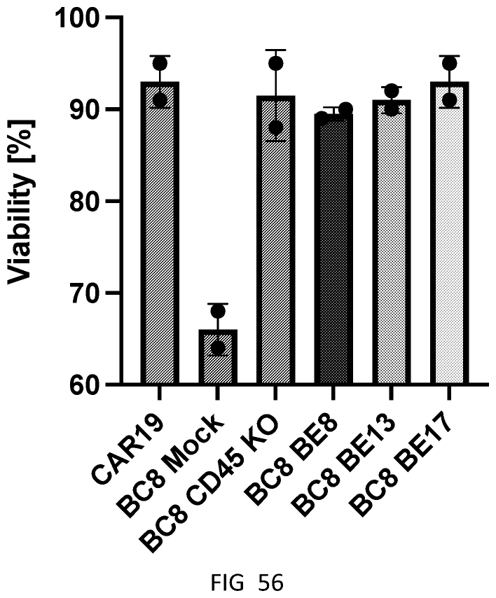

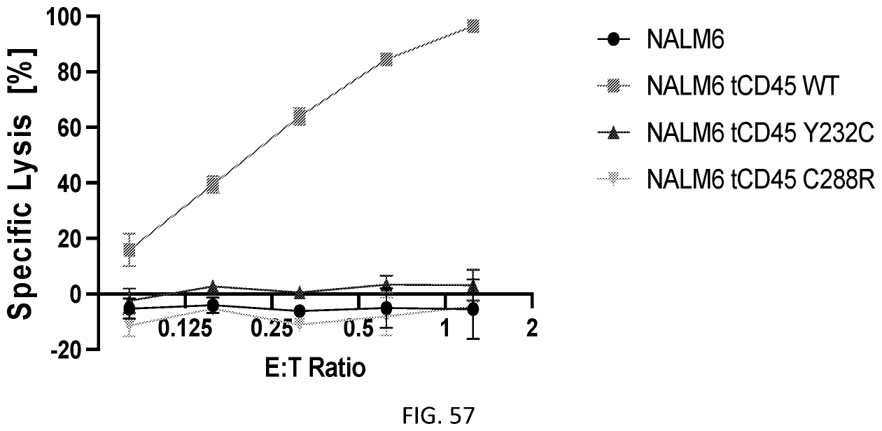

A series of studies was then performed to determine whether base-editing of CD45 prevented epitope masking and CAR internalization during ex vivo expansion of edited cells. FIG. 55 are a series of flow plots and a bar graph showing that the ratio of surface CAR expression to transduced cells in CD45KO and epitope edited treatment groups is 1, suggesting that every transduced T cell expresses the CAR receptor in their surface. In non-edited cells, a large fraction of transduced cells do not express the CAR likely due to epitope masking or CAR internalization from excessive stimulation during manufacturing. To generate these plots, cells were stained with fluorescently labeled recombinant CD45 for surface CAR staining and with anti-NGFR for transduction. Similar studies assessing the viability of base-edited cells following expansion revealed that base-editing and CD45 knockout improves cell viability during CAR-T cell manufacturing, suggesting that fratricide can be prevented by epitope editing (FIG. 56). In order to definitively show that base-edited CD45 is no longer targeted by anti-CD45 CAR T cells, NALM6 target cells were modified to express edited several edited versions of CD45 and then incubated with anti-CD56 CAR expressing T cells. No killing of edited cells was be overserved, while NALM6 cells expressing wildtype CD45 were readily lysed (FIG. 57).

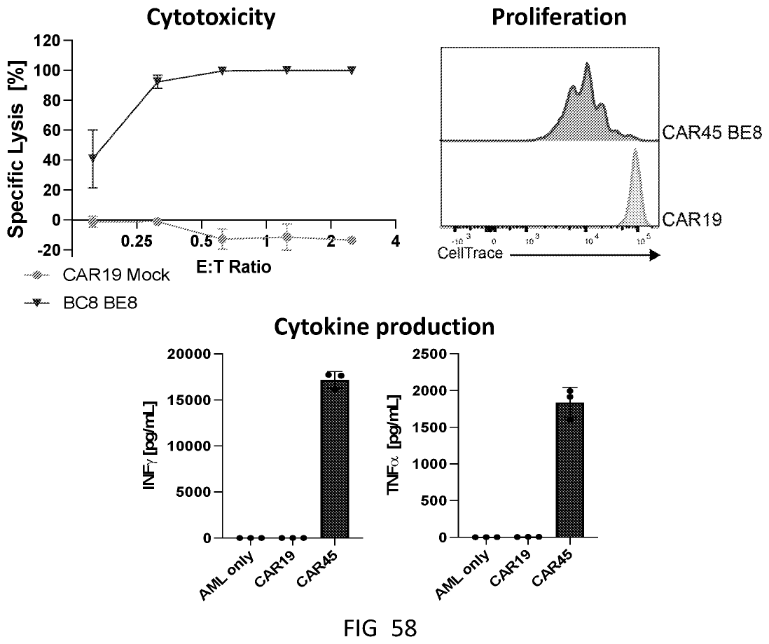

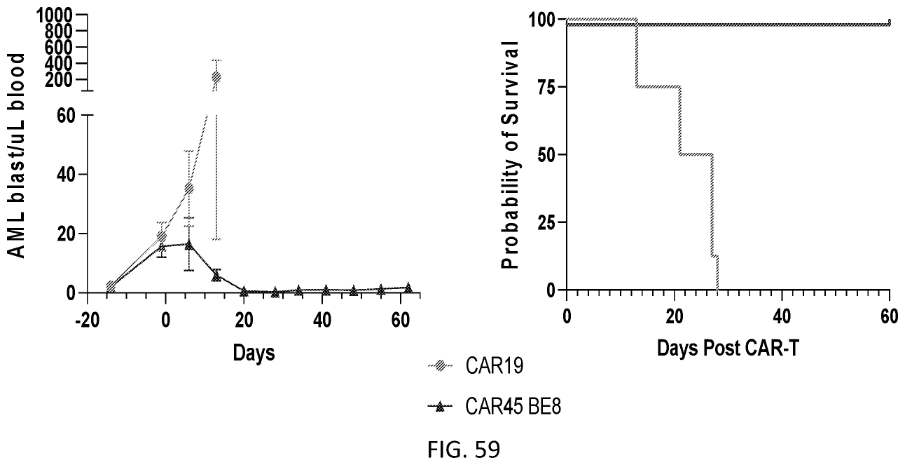

In order to determine whether CD45 epitope-edited cells were capable of normal cytotoxic function, a series of studies were conducted in which edited CAR-T cells were co-cultured with hematologic cancer cell lines and primary cancer cells in vitro. These studies demonstrated that epitope edited CAR T cells can kill AML tumor cells (MOLM14). Further, proliferation studies demonstrated that epitope edited CAR45 are capable of proliferating when cocultured with tumor cells, and that epitope edited CAR45 produce T cell effector cytokines INFg and TNFα upon contact with target cells (FIG. 58). To measure the efficiency of in vivo cytotoxicity of epitope edited CAR45 T cells, patient-derived tumor cells were transplanted into NSG mice. After engraftment, mice were injected with CAR19 (control) and edited CAR45 T cells. Edited CAR45 T cells were effective and clearing the tumor and prolonging survival of mice (FIG. 59).

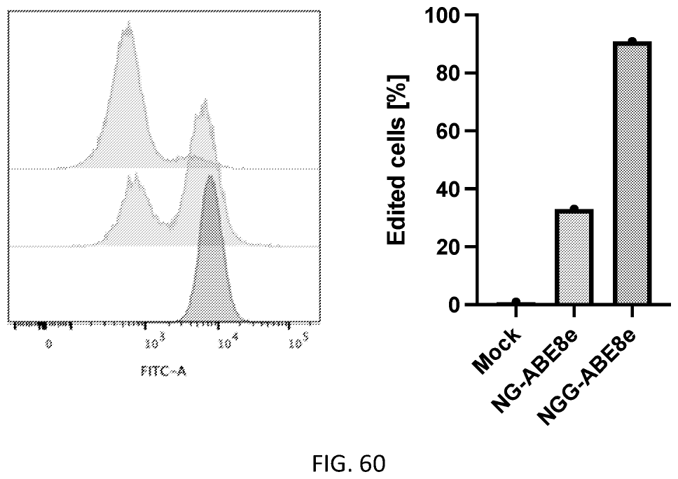

Having demonstrated the efficiency of base-editing in human T cells, studies were then performed which assessed whether this strategy could be used to modify human hematopoietic stem cells (HSCs), as in some embodiments of the invention, the treatment with anti-CD45 CAR is accompanied by the engraftment of CD45-edited HSC's, which reconstitute the subject with blood cells resistant to CAR killing. FIG. 60 shows that epitope editing is effective in human CD34+ HSCs (BE8 is depicted as an example). Editing efficacy can be titrated by using different base editors (NG-SpCas9 or NGG-SpCas9 fused to TadA.

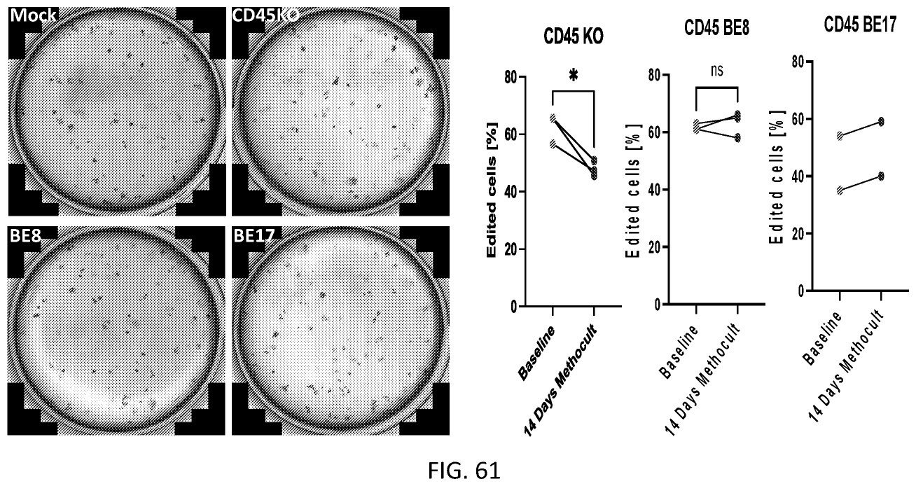

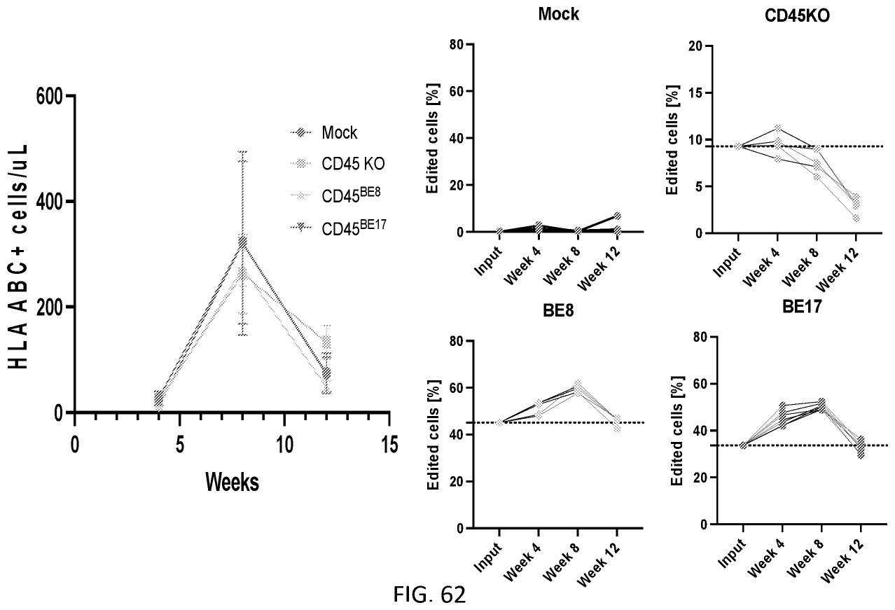

Having proved that HSCs can be base-edited, a follow-up study was conducted to determine whether epitope edited HSCs could proliferate and differentiate in vitro. In FIG. 61, HSCs were edited using NG-ABE8e (BE8 and BE17), standard Cas9 nuclease (CD45KO, or control (Mock). One day after electroporation, HSCs were cultured in semisolid, cytokine rich, methylcellulose media and colonies were quantified and sequenced after 14 days. Left panel: Example pictures showing that edited cells can form colonies. Right panel: Colonies were harvested and sequenced. Edited allele frequency compared to baseline was measured by sequencing, showing that CD45 knockout cells are being selected against, whereas base-edited cells are not. Further, these studies examined whether CD45 base-edited HSCs engraft and replace peripheral blood mononuclear cells in vivo (FIG. 62). Edited HSCs were injected into NSG mice and the number of human cells in the blood, as well as the editing frequency of human cells in the blood were monitored for 3 months. These results demonstrated that HSCs could engraft and persist with similar cell numbers compared to unedited cells. Further, edited allele frequency compared to baseline was measured by flow cytometry, showing that CD45 knockout cells are being selected against, whereas base-edited cells are not.

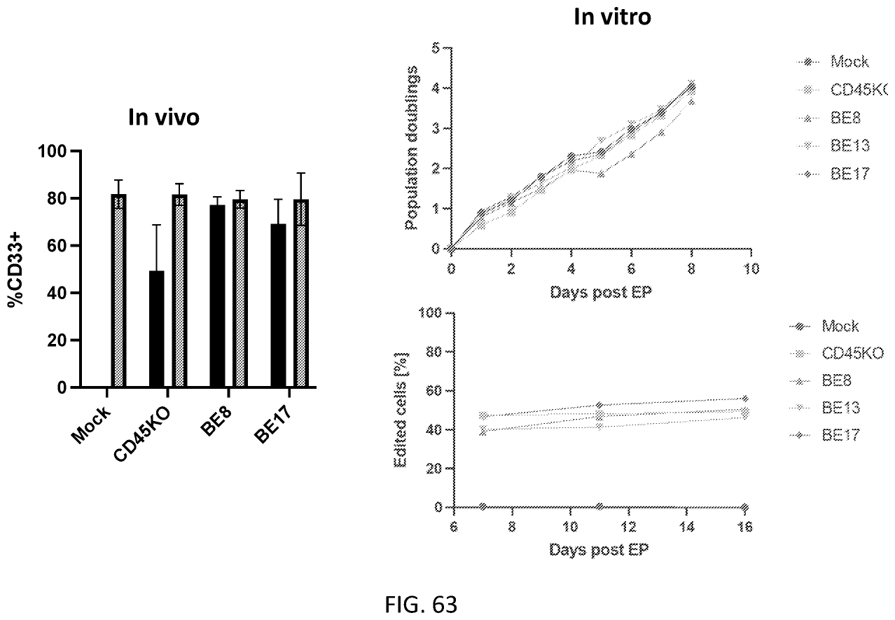

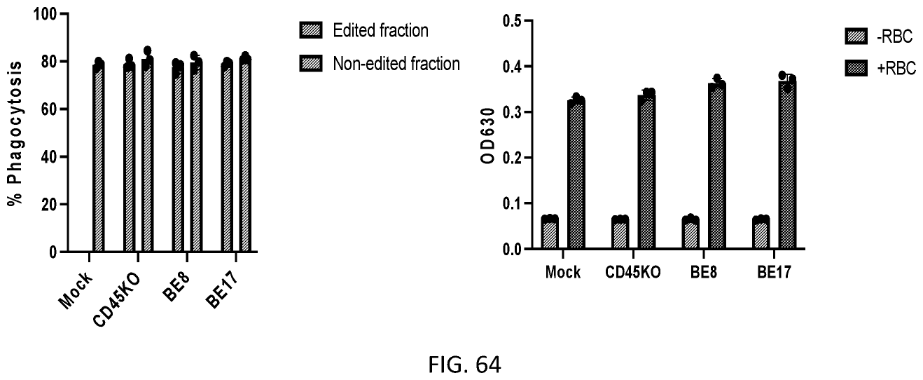

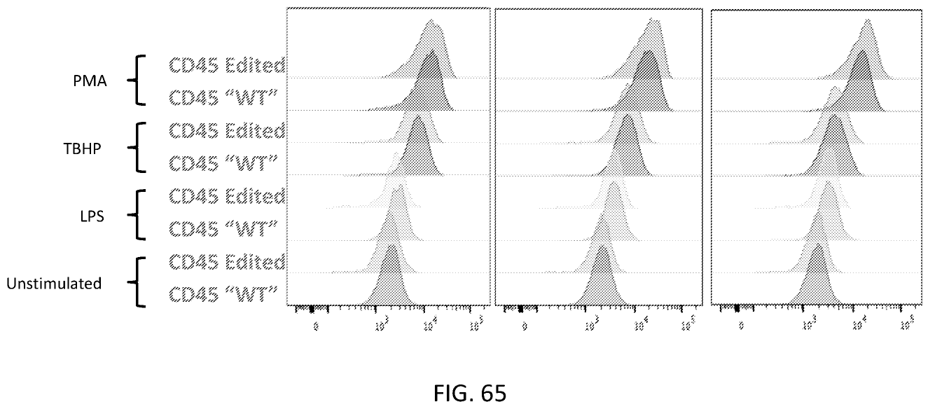

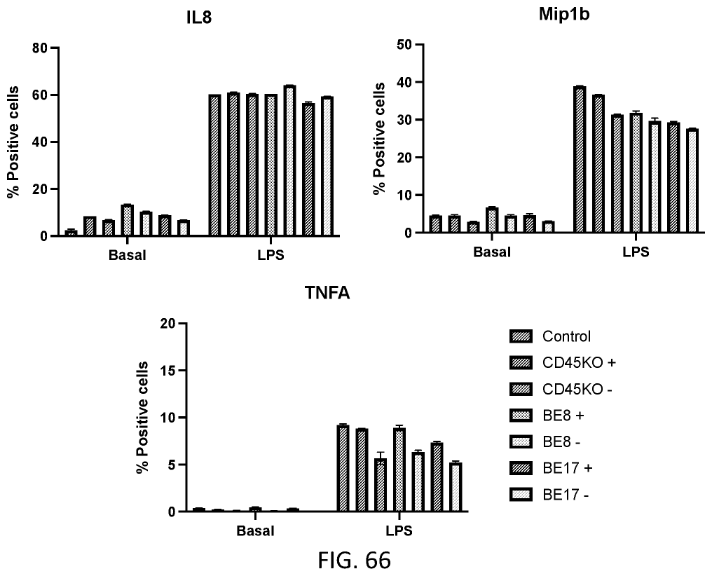

The next set of studies sought to understand whether CD45-edited myeloid cells retain normal function after differentiating from edited HSCs. Mice engrafted with CD33+ edited HSCs were followed to observe blood cell development. FIG. 63 demonstrates that the base-edited HSCs can differentiate into myeloid (CD33+) cells in vivo, and that edited HSCs differentiated and expanded into myeloid cells in cytokine rich media. No difference in cell numbers and editing frequency was observed in base-edited cells. FIG. 63 demonstrates that in vitro differentiated myeloid cells from CD45 edited HSCs can phagocytose E. coli bioparticles and RBC's. Here, differentiated myeloid cells were assessed for their ability to phagocytose bacteria (E. coli) and sheep red blood cells. No differences between edited and non-edited groups were observed, suggesting that phagocytic function is preserved in edited myeloid cells. In a follow-up study, in vitro differentiated myeloid cells were assessed for their ability to produce reactive oxygen species (ROS) after biological or pharmacological stimulation. No differences between edited and non-edited groups were observed, suggesting that production of ROS is preserved in edited myeloid cells (FIG. 65). Likewise, in vitro differentiated myeloid cells were also assessed for their ability to produce effector cytokines at baseline and after LPS stimulation. Similarly, no differences between edited and non-edited groups were observed, suggesting that phagocytic function is preserved in edited myeloid cells (FIG. 66).

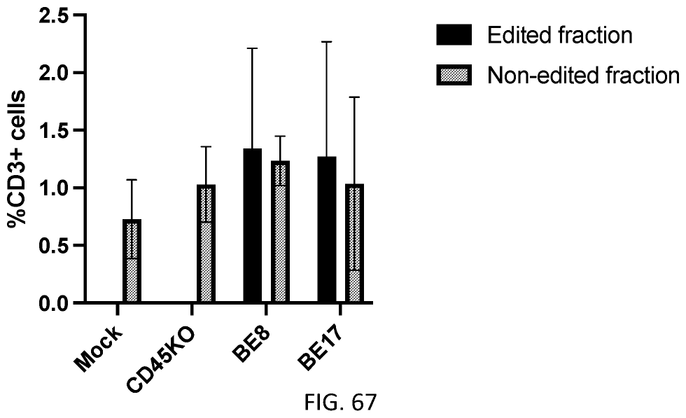

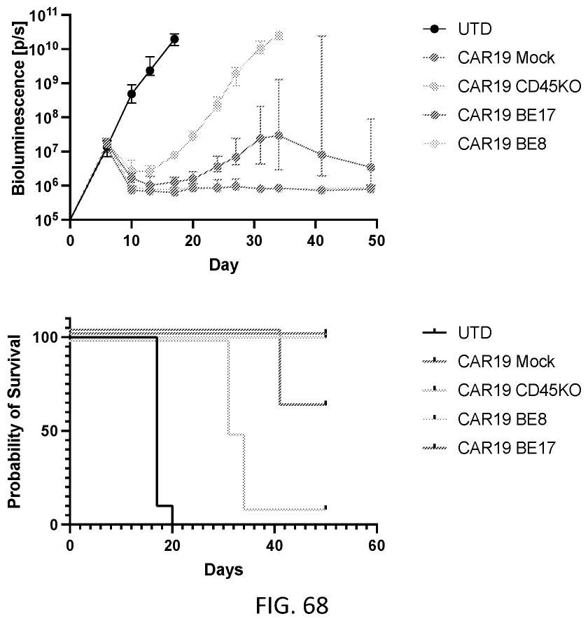

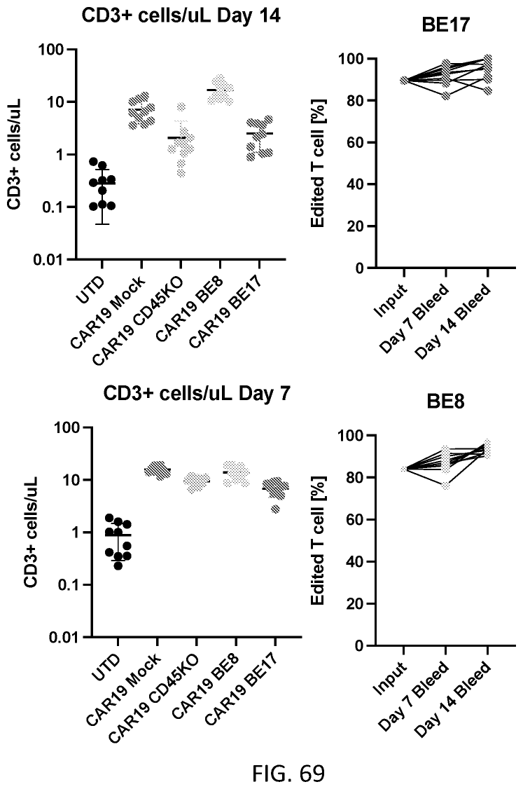

Having assessed myeloid cell function, studies then examined the function of T cells differentiated from CD45-edited HSCs. Here, the frequency of T cells (CD3+) 12 weeks post edited HSC engraftment into mice was assessed. Differentiation of base-edited, but not CD45 knockout HSC into T cells was maintained (FIG. 67). These data are in line with reports that humans with CD45 deficiency do not generate T cells. Further, a subsequent in vivo tumor clearance study demonstrated that CD45KO impairs CAR19 T cells function, while CD45 base-editing does not (FIG. 68). CD45KO CAR19 T cells relapse and succumb to their tumor whereas base-edited CD45 CAR19 T cells are able to control tumor similar to non-edited CAR19 cells. Lastly, these studies found that base-edited T cells expand in the blood while retaining editing frequency (FIG. 69). Here, CD45 edited CAR19 T cells expand in the blood of tumor bearing mice. Mice were blead at 7 and 14 days post CAR T cell injection and numbers of T cells in peripheral blood and the frequency of edited cells were measured.

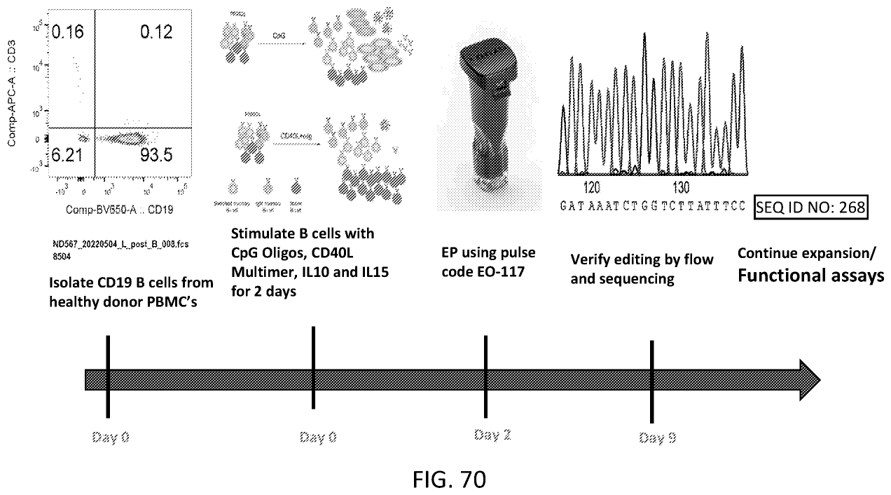

Next, a series of studies was undertaken to assess the effect of CD45 base editing on B cells that differentiate from edited HSCs. Initial studies were conducted in vitro on CD19+ cells isolated from healthy donor PBMC, which were then stimulated for two days prior to electroporation with editing constructs. Seven days after electroporation, editing efficiency was assessed by sequencing. A workflow of these studies is illustrated in FIG. 70.

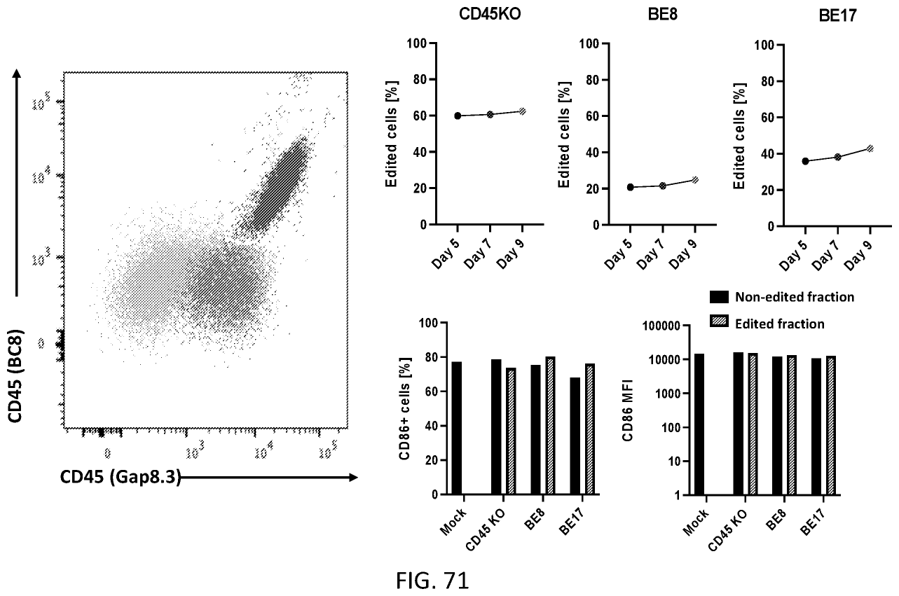

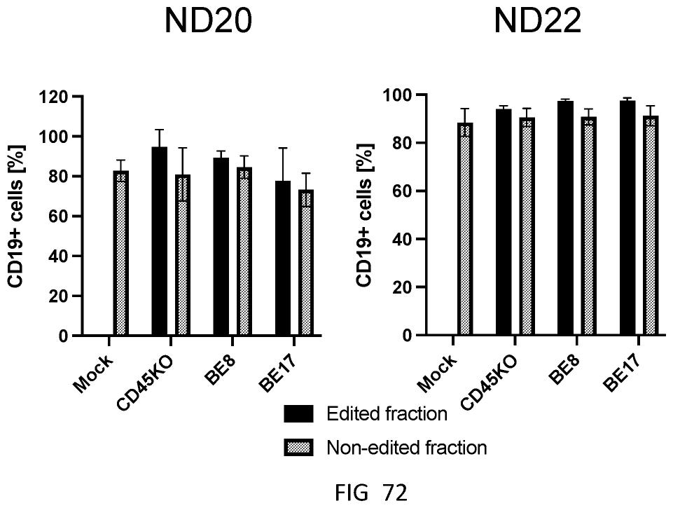

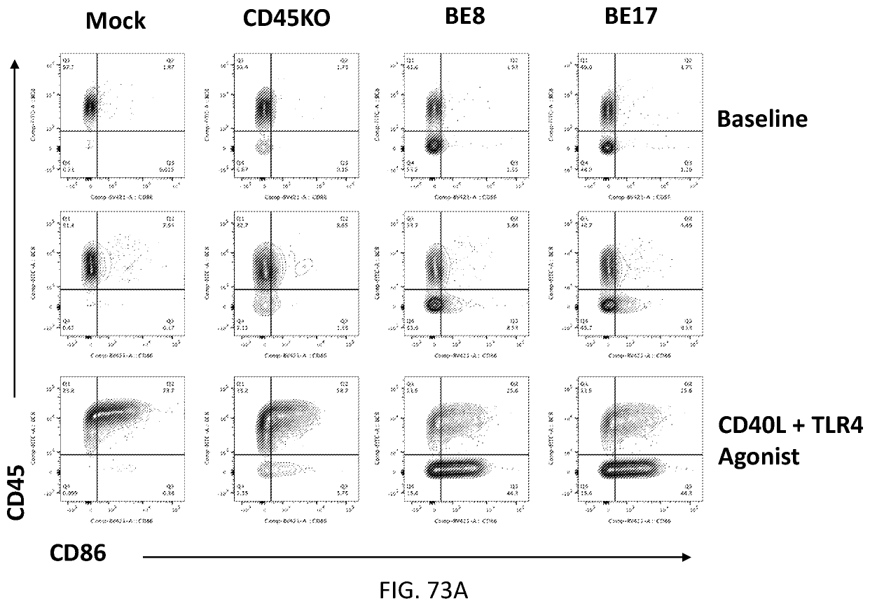

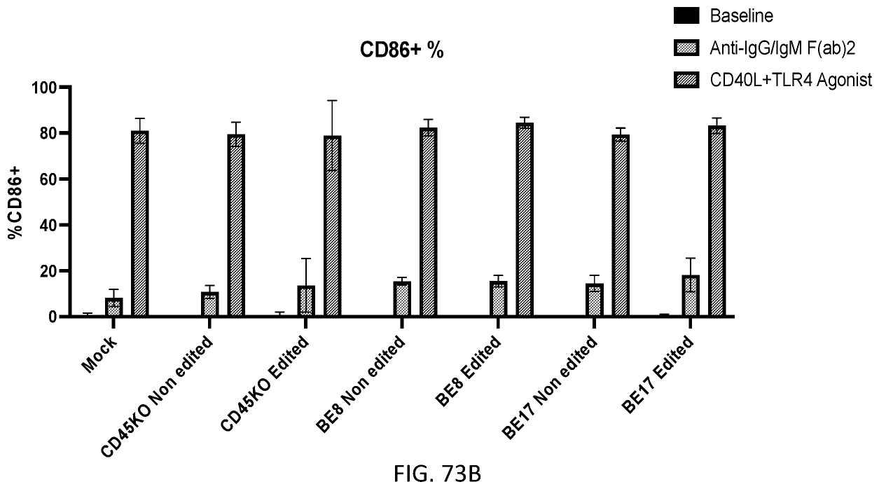

FIG. 71 shows that CD45 base editing does not impair B cell activation/expansion. Flow cytometry staining found that base edited B cells are negative for a target epitope CD45 antibody (BC8) but positive for flow staining with a control antibody (Gap8.3) specific for a nearby but distinct epitope. These studies also found that the frequency of edited cells is stable during ex vivo B cell expansion suggesting that edited B cells are capable of proliferating in response to cytokine/TLR4/CD40 mediated activation. Robust expression of the activation marker CD86 was also found on edited and non-edited B cells. Having determined that mature B cells could be edited ex vivo using the gene editing system of the invention, studies then assessed whether edited HSCs could differentiate into B cells in vivo. FIG. 72 demonstrates that 8 weeks after edited HSC engraftment into NSG mice, differentiation of human HSCs into B cells was verified in the peripheral blood as measured by staining for the CD19 lineage marker. Without wishing to be bound by theory, these results suggested that edited HSCs were capable of differentiating into B cells similar to non-edited cells. To determine whether B cells differentiated from edited HSCs retained normal function, PBMCs from engrafted mice were isolated 10 weeks post engraftment and stimulated ex vivo with anti IgG/IgM f(ab)2 or CD40L and TLR4 agonist. Activation was measured by flow staining for the B cell activation marker CD86. FIG. 73A shows representative flow plots for each condition. FIG. 73B shows quantification of flow plots (n=3). In total, these data demonstrated that in vivo differentiated, edited, B cells can be activated through the BCR and through CD40/TLR4R mediated stimulation.

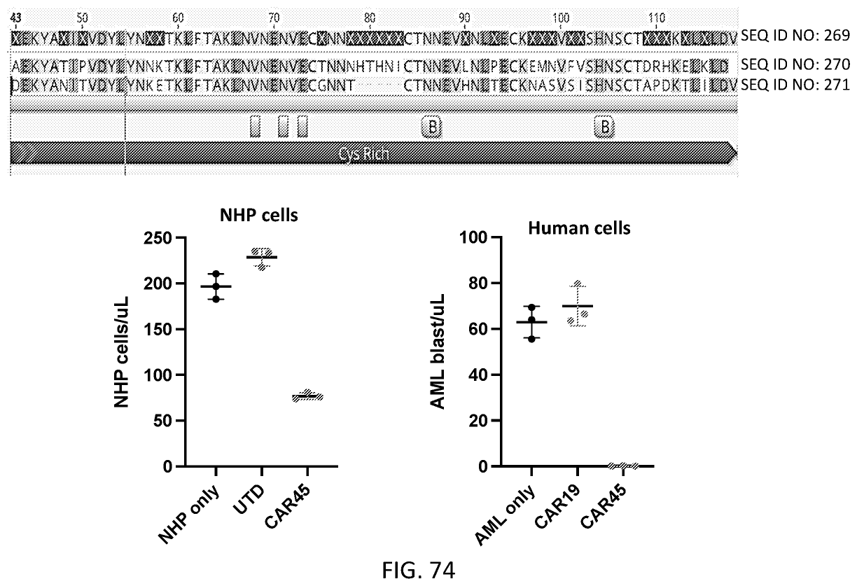

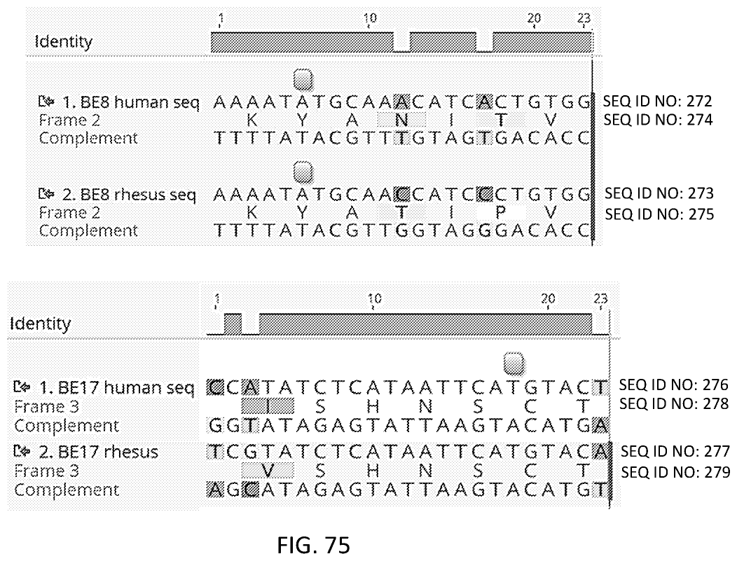

Lastly, a series of studies was undertaken to determine whether the BC8 CD45 CAR construct could cross-react with non-human primate (NHP) CD45. While many epitope specific amino acids are conserved between human and rhesus macaque, only 74% overall amino acid sequence identity exists in this domain (see FIG. 74, top). FIG. 74, bottom demonstrates that BC8 interaction with rhesus macaque CD45 seems to be weaker compared to human CD45 based on MFI and functional readout but the results are clearly above baseline. Lastly, sequence data suggests that editing of the NHP CD45 locus with the constructs developed for human cells is possible. The BE8 NGG PAM and target adenine is conserved but sgRNA has to be adjusted for rhesus CD45, while the NGG PAM for BE17 not conserved in rhesus but could still be edited with NG-restricted BE since target adenine is conserved (FIG. 75).

### Example 5: Development of a CD45RO Specific CAR

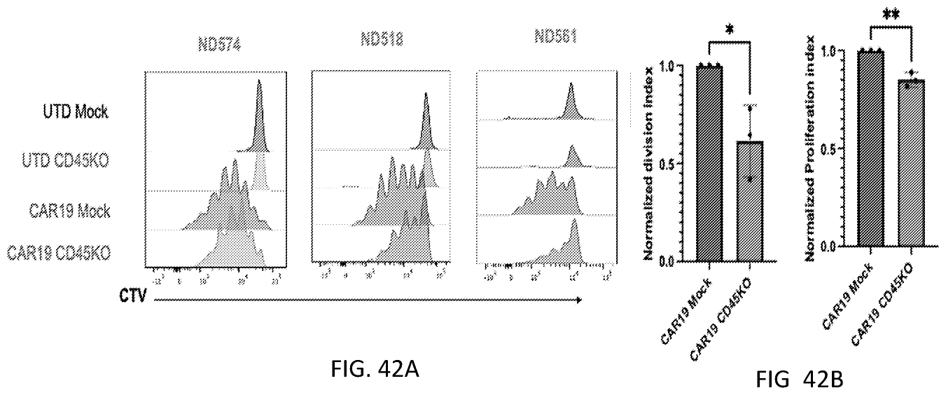

A CAR construct was also generated that is specific for an isoform of CD45 that is expressed when exons 4, 5, and 6 are removed via splicing, which brings the distal N-terminal domain in proximity with the membrane distal part of the mucin like domain. This splice event forms an epitope that is recognized by an antibody clone called UCHL-1, which is dependent on an O-glycosylation on Thr8. By mutating this Thr to an alanine (which is technically achievable with current gene-editing technologies), the antibody clone fails to recognize CD45RO T8A (shown in FIG. 42, left). CD45RO specific CAR-T cells were expanded and found to be effective at eliminating CD45RO positive cells as this population is deleted in CD45RO CAR transduced groups as compared to non-transduced or CAR19 transduced cells (FIG. 42, right).

### Example 6: Redundancy of CD45 Function in Primary Human T Cells

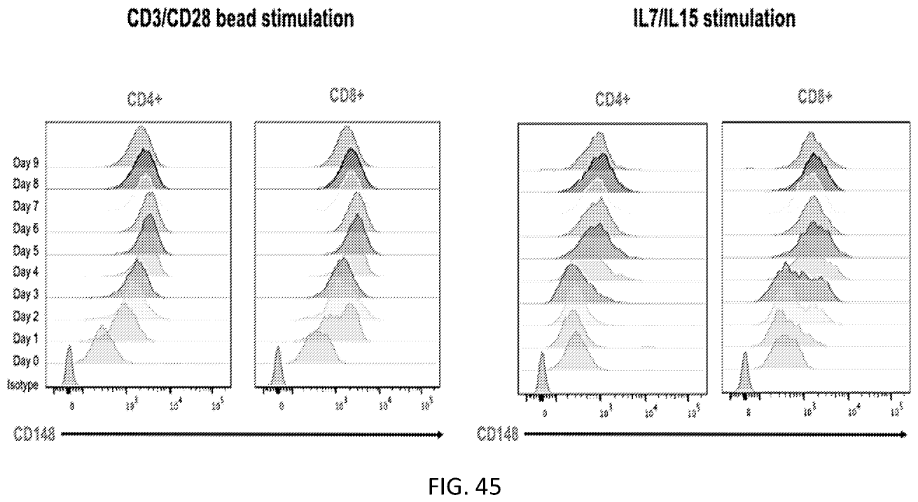

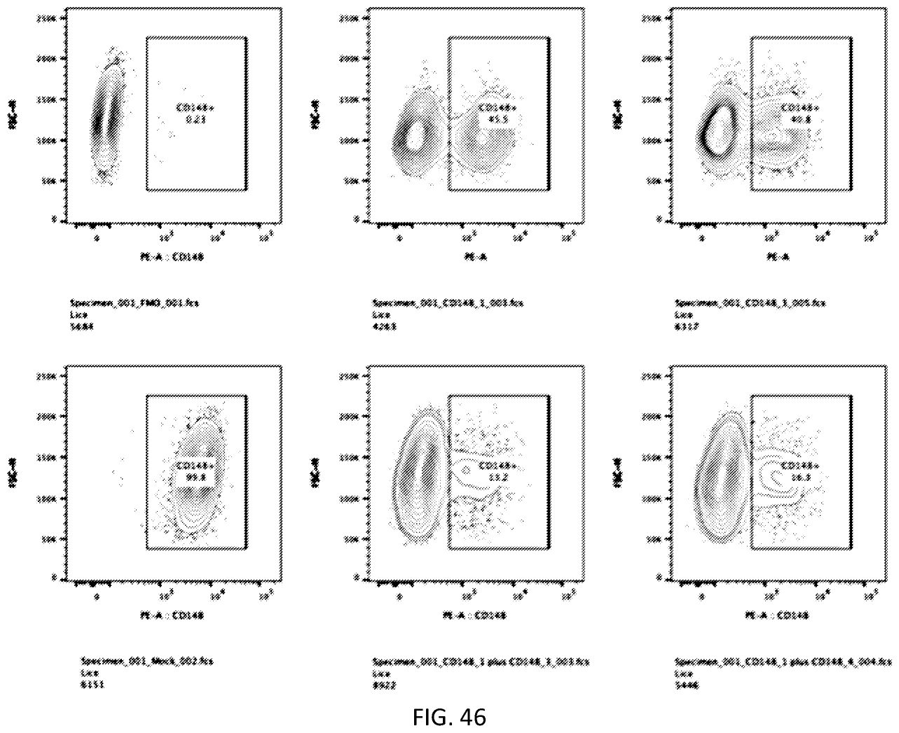

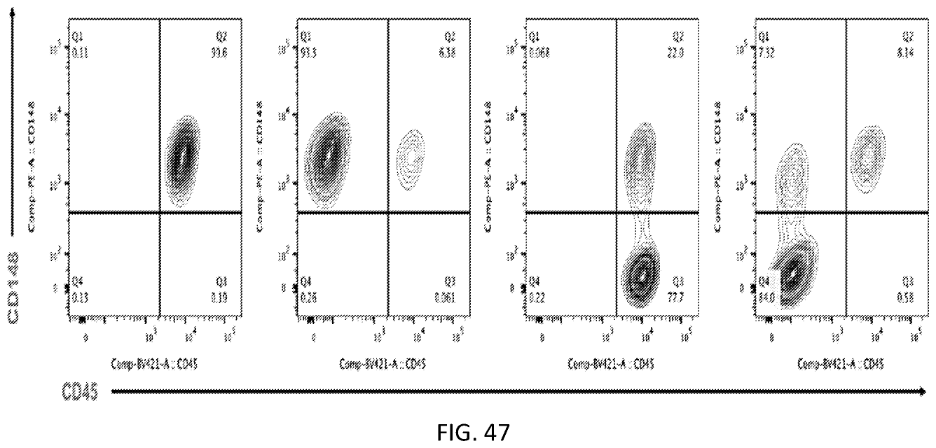

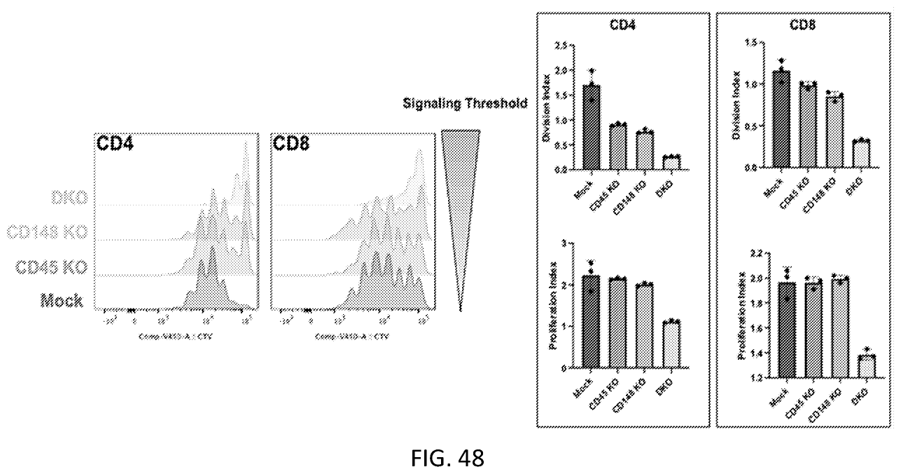

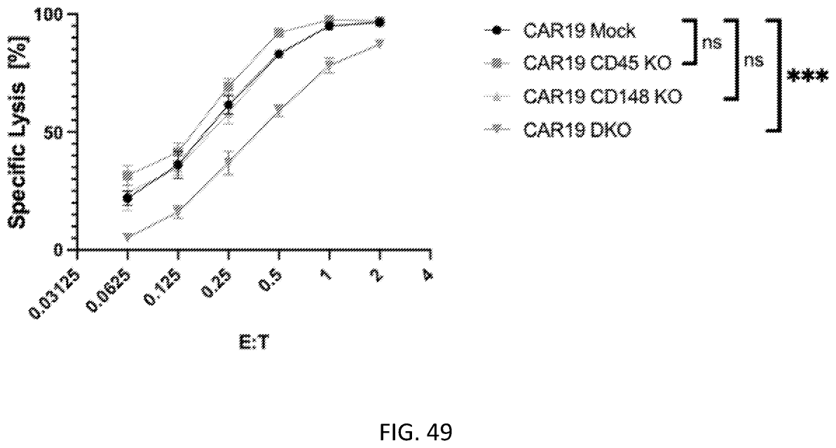

Previous studies in the present disclosure highlighted differences between primary human primary T cells and Jurkat T cells in that ablation of CD45 expression in Jurkat cells resulted in a more profound inhibition of function as compared to primary T cells. One possible reason for this is that human T cells express a phosphatase called CD148 that could act to be functionally redundant to CD45. Notably CD148 is absent in Jurkat cells (FIG. 45B) and its transduction into CD45 deficient cells rescues their function (FIG. 45C). In vitro stimulation studies demonstrated that CD148 is expressed on naïve human T cells and expression increases following T cell activation, which is a key part of the CAR T cell production (FIG. 46). To determine a role for CD148 in providing redundancy for CD45 in T cells, two gRNAs were developed that target and knockout CD148 in human T cells with high efficiency (FIG. 47). These gRNAs were combined with existing CD45 targeting gRNAs in order to generate CD45/CD148 double-knockout cells (FIG. 48). Subsequent in vitro proliferation and cell lysis studies demonstrated that these double-knockout cells are unable to both proliferate (FIG. 49) and lyse target cells (FIG. 50). In total, these data demonstrate that CD148 expression provides a significant amount of redundancy in human T cells and, without wishing to be bound by theory, suggests that modifying CD45 in primary human T cells would not greatly affect cytotoxic function even if the genetic alteration do reduce the signaling function of CD45 protein.

### Example 7: Multiplex Base Editing CD45 and CCR5 to Eliminate Latent HIV Reservoirs after AutoHSCT

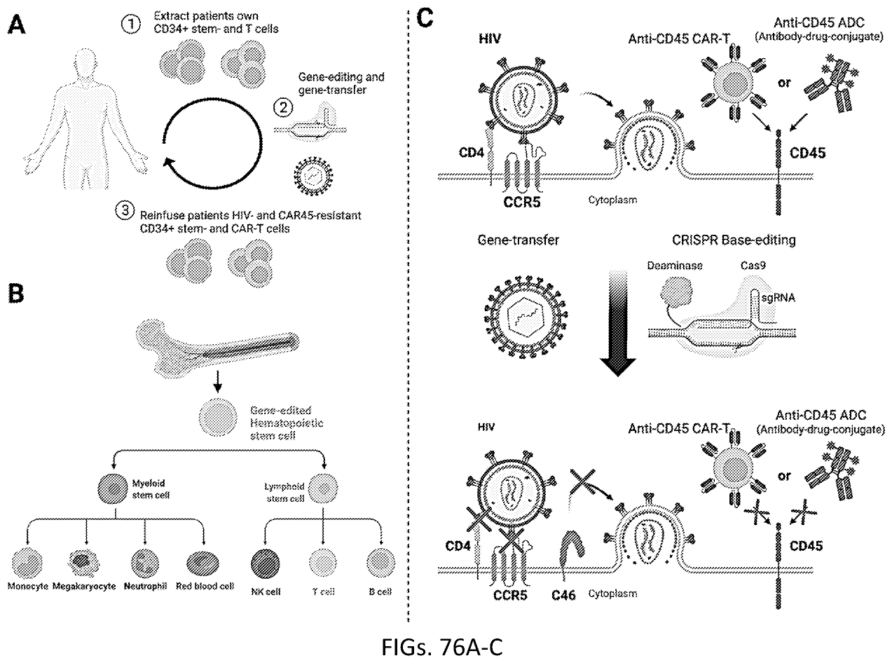

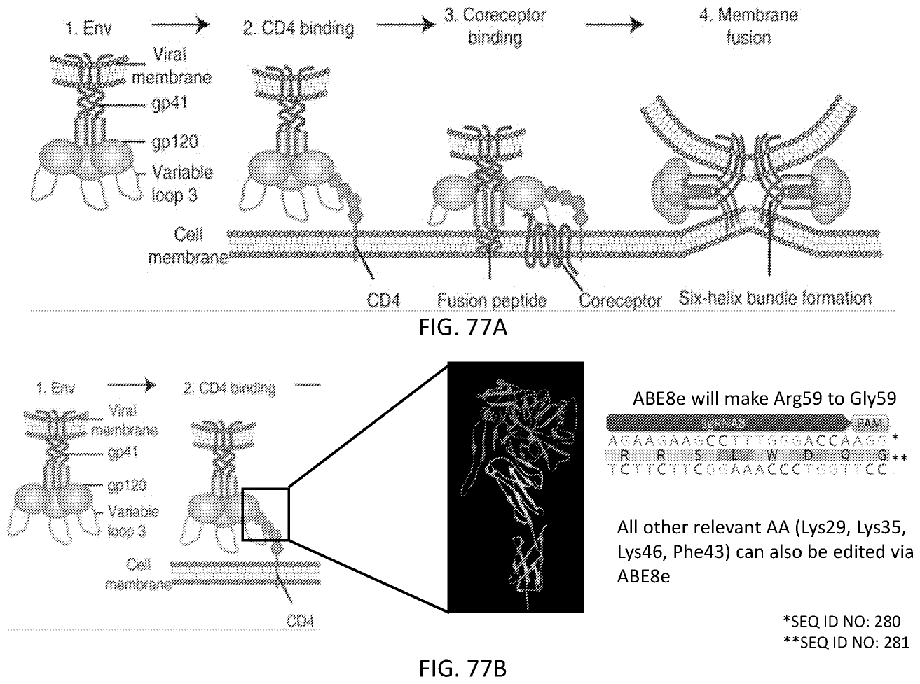

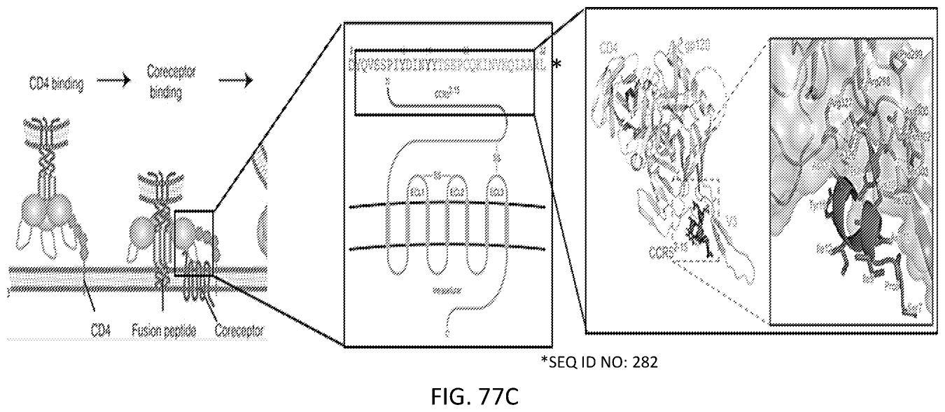

The purpose of these studies was to determine whether CD45/CCR5 edited CAR45 T cells could eliminate latent HIV reservoirs while CD45/CCR5 engineered HSCs, which are protected from HIV re-entry, can repopulate human blood. A strategy which is illustrated in FIGS. 76A-76C. HIV Env proteins require initial binding to CD4 followed by co-receptor binding (either CCR5 or CXCR4) to enter human cells, and preventing binding of HIV Env proteins to these receptors has been shown to confer resistance to HIV infection in human cells by preventing HIV cell entry (illustrated in FIG. 77A). Genetic deletion of human receptors involved in HIV env binding would require knockout of three genes (CD4, CCR5 and CXCR4) in human cells. While this can be done easily by conventional CRISPR/Cas9 technics, inducing multiple DSB's increase the risk of chromosomal translocations among other undesirable outcomes. Genetic deletion of these genes can be achieved without DSB's by base-editing start/stop codons or splice-donor/acceptor sites. However, all three genes have relevant cellular functions and deficiency may lead to serious disease (CXCR4 deficiency causes WHIMP syndrome).

Therefore, the focus of this example is the strategy of editing HIV receptor binding sites without affecting the biological function of the receptor proteins themselves. Amino acid residues on CD4, CCR5 and CXCR4 involved in HIV env protein binding are known through genetic polymorphism studies (eg. CCR5A32), mutagenesis of CD4/CCR5/CXCR4 (eg. alanine mutagenesis), and structural data (eg. x-ray/cryo-em). Editing amino acid residues in CD4, CCR5, and CXCR4 that bind to HIV env gp120 would render cells resistant to HIV entry without losing expression of these proteins.

A key receptor for the binding of HIV to a target cells is CD4. HIV gp120 binds to most N-terminal of the four Ig-like domains of CD4, with several key amino acids known to regulate this process: AA40-48, which in general make up 63% of all interactions between CD4 and gp120 (Kwong et al. Nature (1998). Of these, Phe43 (exposed hydrophobic)->F43A and F43I reduced gp120 binding 500-fold but substitutions with Tyr, Trp or Leu had only small effects (Moebius et al. J. Exp. Med 1992). Likewise, Lys29, Lys35, Lys46, Arg59 (+ charged)->changing them reduced binding 7-50-fold (Moebius et al. J. Exp. Med 1992) (illustrated in FIG. 77B).

Another target of HIV binding is CCR5. Here, HIV gp120 binds to amino acids located at N-terminal portion of this protein (AA2-31) and a second extracellular loop. Amino acids in CCR5 that are important for binding of gp120 include sulfotyrosines: Tyr3, Tyr10, Tyr14, Tyr15, serines (presumably through O-glycosylation): Ser6, Ser7, Ser17, and other negatively charged residues that bind to positive gp120 pocket: Asp2, Asp11, Glu18, as well as AA in the extracellular loop: Lys197, Asp276 Others: C20, Ile9, Gln21, Lys22 (see FIG. 77C).

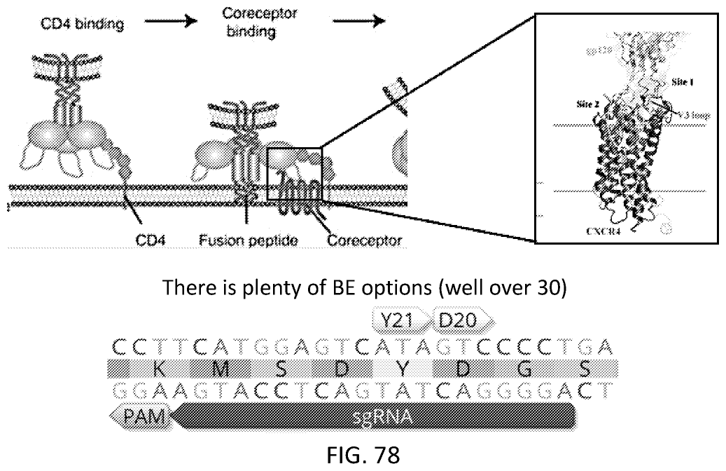

Another key HIV receptor is CXCR4. Here, HIV gp120 binds to amino acids located at N-terminal portion of CXCR4 and a second extracellular loop (similar to CCR5). Key amino acids include sulfotyrosines Tyr7, Tyr12, Tyr21, serines (presumably through O-glycosylation), as well as other negatively charged residues that bind to positive gp120 pocket: Asp10, Asp20, Asp22, Glu26, and AA in extracellular loop: Arg183, Asp187, Arg188, Asp193, Asp262, E268, D182, D187, F189, and P191. Also important are TM domain AA implicated in HIV entry: Tyr45, His79, Asp97, Pro163, Trp252, Tyr255, Asp262, Glu288, His294, and Asn298. Overall, there are at least 30 amino acids which would make effective targets for base editing in order to reduce the ability of gp120 to bind to this receptor protein (see FIG. 78).

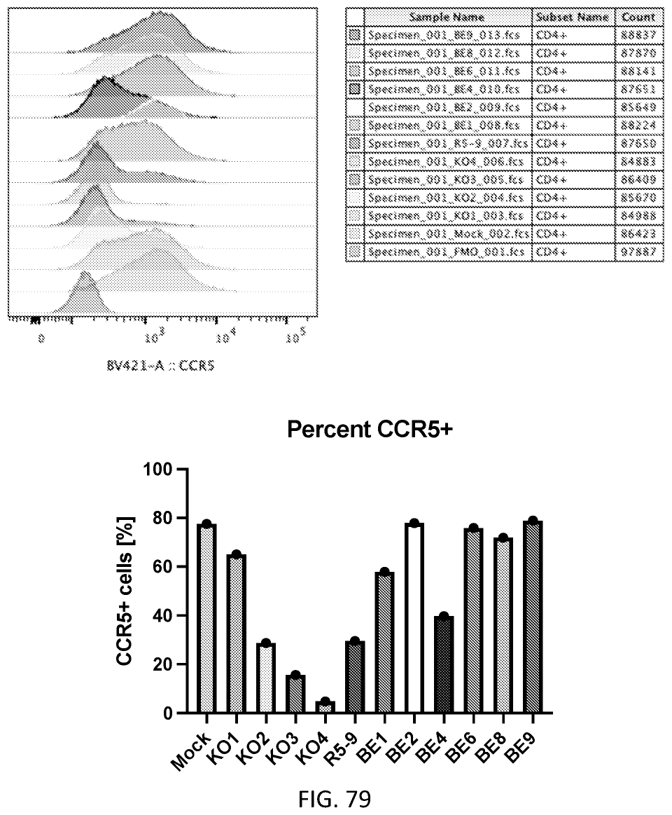

To demonstrate one embodiment of the invention, base editing of CCR5 was assessed to disrupt gp120 binding using CRISPR/Cas nucleases and adenine base editing. A series of sgRNAs were created that target CCR5 and a screen was conducted to identify sgRNAs that abolish CCR5 expression or mutate HIV docking site in the CCR5 gene. FIG. 79, top shows CD4+ cells that were activated and electroporated with either conventional Cas9 nuclease or ABE8 and various sgRNAs. Several hits from the gRNA screen demonstrate effective abrogation of CCR5 expression with either base-editing or CRISPR/Cas nucleases. Some edits (for example BE1) are effectively base-edited while leaving CCR5 expression mostly intact. These studies identified 4 sgRNAs which target CCR5 expression (KO 1-4) through CRISPR/Cas9 knockout, 1 whose base-editing function knocks out expression by removing the start codon (R5-9), and 6 which base-edit residues that bind to gp120 (BE1, BE2, BE4, BE6, BE8, and BE9).

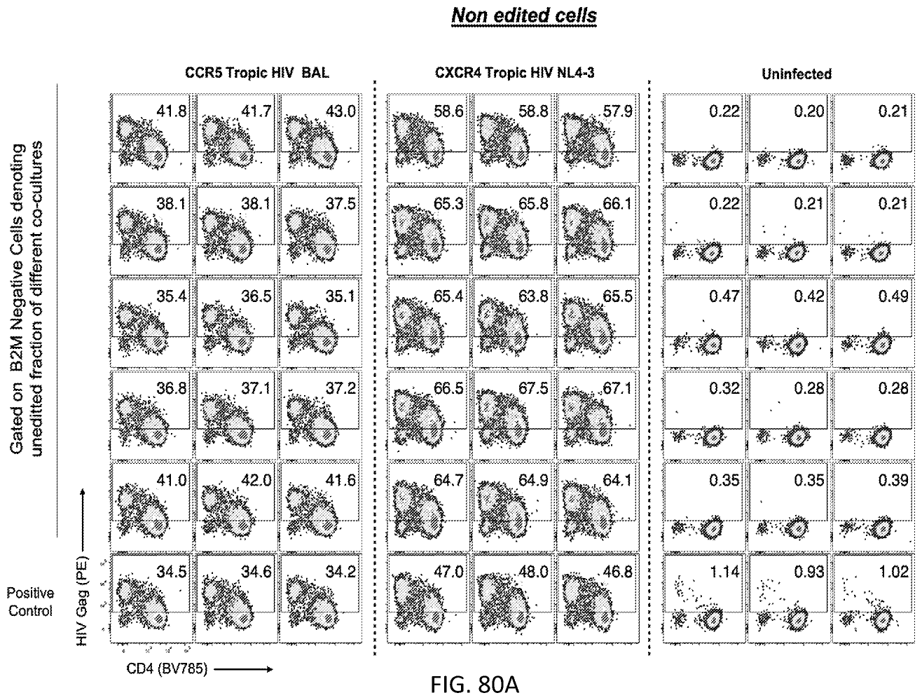

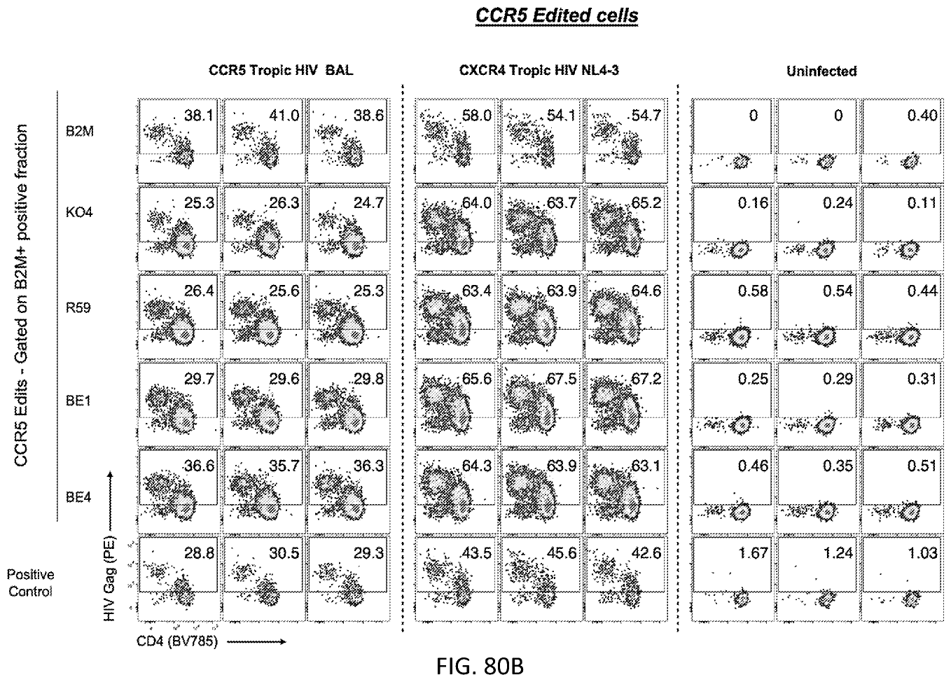

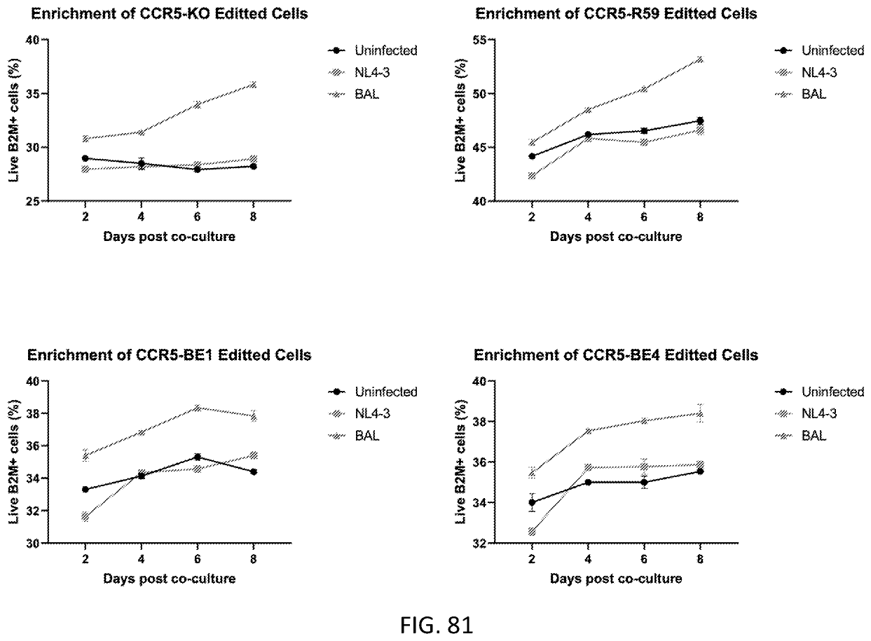

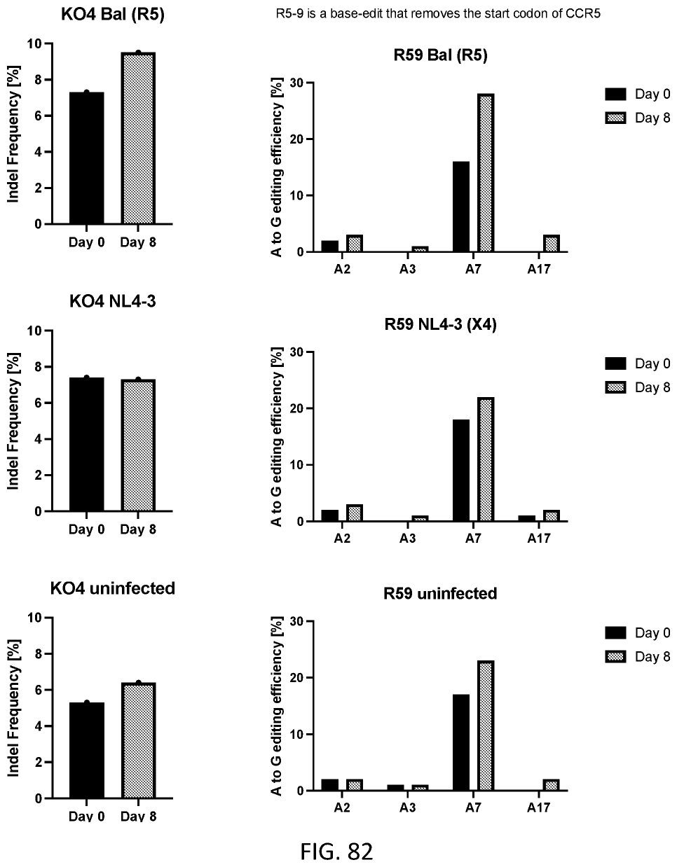

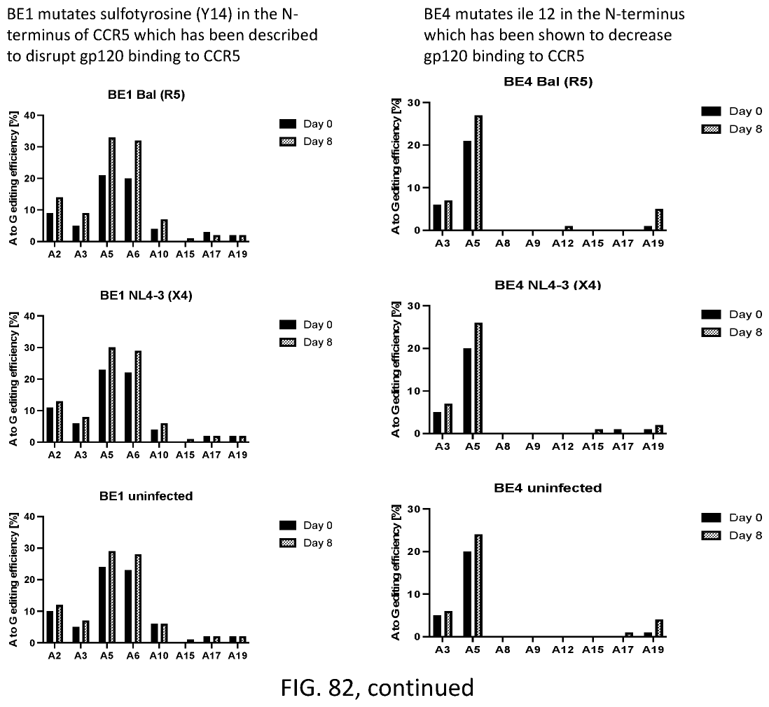

Base edited or non-edited cells were then challenged with infection by R5 tropic or X4 tropic HIV virus and infection was measured after 6 days by staining for HIV gag by flow cytometry. These results demonstrated that CCR5 edited cells were protected from R5 tropic HIV virus infection compared to control cells as measured by a decrease in gag+ cells (FIGS. 80A-80B). Since HIV infection kills infected T cells, enrichment of CCR5 edited cells was measured over the course of infection. As expected, CCR5 edited cells became enriched over time when infected with R5 tropic HIV virus (BAL). Enrichment was indirectly measured by staining for live, B2M positive cells (as CCR5 non-edited cells were knocked out for B2M) (FIG. 81). Lastly, amplicon sequencing of cells after infection revealed enrichment of edited CCR5 alleles during infection, implying resistance to HIV infection mediated cell death (FIG. 82). Without wishing to be bound by theory, in total these results suggest that CCR5 base editing can be used to generate T cells which are resistant to HIV infection.

### Example 8: Strategies for Base Editing of CD33

The cancer treatment strategy of combining CAR-T targeting of a cancer-associated antigen with base editing of the endogenous epitope of the CAR-T cells disclosed in the previous examples can also be used to treat CD33-expressing cancers, especially acute myeloid leukemia (AML), among others. CD33 is a myeloid differentiation antigen that is normally expressed by myeloid stem cells, myeloblasts and monoblasts, monocytes and macrophages, granulocyte precursors, and mast cells. While normally associated with AML (more than 80% of cases), aberrant CD33 expression can also be found on some B-lymphoblastic and T-lymphoblastic leukemias and lymphomas. Due it it's expression on many normal myeloid cells and precursors, the targeting of CD33 with anti-cancer therapies, including CAR T cells can be associated with significant off-target toxicity, as normal cell populations are depleted along with the CD33-expressing tumor cells.

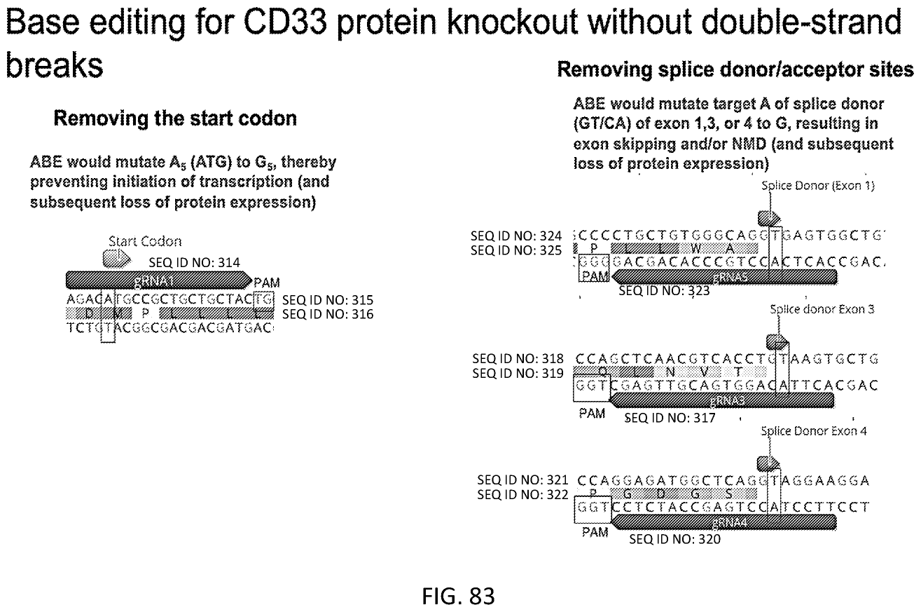

The base-editing strategy of the invention of the current disclosure can be used to edit or knock-out expression of CD33 in HSCs prior to transplant without the induction of double-strand breaks (DSBs). FIG. 83 illustrates four gRNAs capable of knocking-out CD33 expression. gRNA1 mutates the start codon (ATG) to a (GTG), thereby preventing initiation of transcription and resulting in the subsequent loss of protein expression. gRNAs 3, 4, and 5 mutate the target A of splice donor sites of exons 1 (gRNA5), 3 (gRNA3), or 4 (gRNA4), resulting in exon skipping and/or nonsense-mediated decay (NMD) which leads to subsequent loss of protein expression). As in previous examples, edited HSCs could be delivered via transplant along with or before/after treatment with anti-CD33 CAR T cells in order to reconstitute the patient's hematopoietic system with cells that are resistant to recognition by any residual CAR-expressing T cells.

### Enumerated Embodiments

The following enumerated embodiments are provided, the numbering of which is not to be construed as designating levels of importance.

Embodiment 1 provides a chimeric antigen receptor (CAR) comprising an antigen binding domain, a transmembrane domain, and an intracellular domain, wherein the antigen binding domain comprises:
i. a heavy chain variable region that comprises three heavy chain complementarity determining regions (HCDRs), wherein HCDR1 comprises an amino acid sequence selected from the group consisting of SEQ ID NOs: 89, 92, 95, 98, 101, 104, 107, 110, 113, and 116, HCDDR2 comprises an amino acid sequence selected from the group consisting of SEQ ID NOs: 90, 93, 96, 99, 102, 105, 108, 111, 114, and 117, and HCDR3 comprises and amino acid sequence selected from the group comprising SEQ ID NOs: 91, 94, 97, 100, 103, 106, 109, 112, 115, and 118; and
ii. a light chain variable region that comprises three light chain complementarity determining regions (LCDRs), wherein LCDR1 comprises an amino acid sequence selected from the group consisting of SEQ ID NOs: 119, 122, 125, 128, 131, 134, 137, 140, and 143, LCDR2 comprises and amino acid sequence selected from the group consisting of SEQ ID NOs: 120, 123, 126, 129, 132, 135, 138, 141, and 144, and LCDR3 comprises an amino acid sequence selected from the group consisting of SEQ ID NOs: 121, 124, 127, 130, 133, 136, 139, 142, and 145.

Embodiment 2 provides the CAR of embodiment 1, wherein the CAR binds CD45.

Embodiment 3 provides the CAR of embodiment 1, wherein the antigen binding domain comprises an antibody or an antigen-binding fragment thereof.

Embodiment 4 provides the CAR of embodiment 3, wherein the antigen-binding fragment is selected from the group consisting of a Fab, a single-chain variable fragment (scFv), or a single-domain antibody.

Embodiment 5 provides the CAR of embodiment 4, wherein the antigen-binding fragment is a scFv.

Embodiment 6 provides the CAR of any one of embodiments 1-5, wherein the antigen binding domain comprises a heavy chain variable region comprising an amino acid sequence having at least 95%-99% identity to the amino acid sequence of any of the heavy chain variable regions set forth in SEQ ID NOs: 146-155.

Embodiment 7 provides the CAR of any one of embodiments 1-6, wherein the antigen binding domain comprises a heavy chain variable region comprising an amino acid sequence of any of the heavy chain variable regions set forth in SEQ ID NOs: 146-155.

Embodiment 8 provides the CAR of any one of embodiments 1-6, wherein the antigen binding domain comprises a heavy chain variable region consisting of an amino acid sequence of any of the heavy chain variable regions set forth in SEQ ID NOs: 146-155.

Embodiment 9 provides the CAR of any one of embodiments 1-8, wherein the antigen binding domain comprises a light chain variable region comprising an amino acid sequence having at least 95-99% identity to the amino acid sequence of any of the light chain variable regions set forth in SEQ ID NOs: 156-165.

Embodiment 10 provides the CAR of any one of embodiments 1-9, wherein the antigen binding domain comprises a light chain variable region comprising an amino acid sequence of any of the light chain variable regions set forth in SEQ ID NOs: 156-165.

Embodiment 11 provides the CAR of any one of embodiments 1-10, wherein the antigen binding domain comprises a light chain variable region consisting of an amino acid sequence of any of the light chain variable regions set forth in SEQ ID NOs: 156-165.

Embodiment 12 provides a chimeric antigen receptor (CAR) comprising an antigen binding domain, a transmembrane domain, and an intracellular domain, wherein the antigen binding domain comprises a heavy chain variable region comprising any of the amino acid sequences set forth in SEQ ID NO: 146-155; and a light chain variable region comprising any of the amino acid sequences set forth in SEQ ID NO: 156-165.

Embodiment 13 provides a chimeric antigen receptor (CAR) comprising an antigen binding domain, a transmembrane domain, and an intracellular signaling domain, wherein the antigen binding domain comprises any one of the amino acid sequences set forth in SEQ ID NOs: 175, 177, 179, 181, 183, 185, 187, 189, 191, 193, 195, 197, 199, 201, 203, 205, 207, 209, and 211.

Embodiment 14 provides the CAR of any one of embodiments 1-13, wherein the transmembrane domain is selected from the group consisting of an artificial hydrophobic sequence, and a transmembrane domain of a type I transmembrane protein, an alpha, beta, or zeta chain of a T cell receptor, CD28, CD3 epsilon, CD45, CD4, CD5, CD8, CD9, CD16, CD22, CD33, CD37, CD64, CD80, CD86, OX40 (CD134), 4-1BB (CD137), and CD154.

Embodiment 15 provides the CAR of any one of embodiments 1-14, wherein the transmembrane domain comprises a transmembrane domain of CD8 comprising an amino acid sequence set forth in SEQ ID NO: 169.

Embodiment 16 provides the CAR of any one of embodiments 1-15, further comprising a hinge domain.

Embodiment 17 provides the CAR of embodiment 16, wherein the hinge domain is selected from the group consisting of an Fc fragment of an antibody, a hinge region of an antibody, a CH2 region of an antibody, a CH3 region of an antibody, an artificial hinge domain, a hinge comprising an amino acid sequence of a CD8 hinge, or any combination thereof.

Embodiment 18 provides the CAR of embodiment 17, wherein the hinge domain is a CD8 hinge comprising an amino acid sequence set forth in SEQ ID NO: 168.

Embodiment 19 provides the CAR of any one of embodiments 1-18, wherein the intracellular domain comprises a costimulatory signaling domain and an intracellular signaling domain.

Embodiment 20 provides the CAR of embodiment 19, wherein the costimulatory signaling domain comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFI-II, Fas, CD30, CD40, ICOS, NKG2C, and B7-H3 (CD276), or a variant thereof.

Embodiment 21 provides the CAR of embodiment 19 or 20, wherein the costimulatory signaling domain comprises a costimulatory domain of 4-1BB comprising an amino acid sequence set forth in SEQ ID NO: 170.

Embodiment 22 provides the CAR of any one of embodiments 1-21, wherein the intracellular signaling domain comprises an intracellular domain selected from the group consisting of cytoplasmic signaling domains of a human CD3 zeta chain (CD3ζ), FeyRIII, FesRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

Embodiment 23 provides the CAR of any one of embodiments 69-72, wherein the intracellular signaling domain comprises an intracellular domain of CD3ζ.

Embodiment 24 provides the CAR of embodiment 23, wherein the CD3ζ intracellular domain comprises an amino acid sequence set forth in SEQ ID NO: 171.

Embodiment 25 provides a chimeric antigen receptor (CAR) comprising:
i. an antigen binding domain comprising: a heavy chain variable region that comprises three heavy chain complementarity determining regions (HCDRs), wherein HCDR1 comprises an amino acid sequence selected from the group consisting of SEQ ID NOs: 89, 92, 95, 98, 101, 104, 107, 110, 113, and 116, HCDDR2 comprises an amino acid sequence selected from the group consisting of SEQ ID NOs: 90, 93, 96, 99, 102, 105, 108, 111, 114, and 117, and HCDR3 comprises and amino acid sequence selected from the group comprising SEQ ID NOs: 91, 94, 97, 100, 103, 106, 109, 112, 115, and 118; and a light chain variable region that comprises three light chain complementarity determining regions (LCDRs), wherein LCDR1 comprises an amino acid sequence selected from the group consisting of SEQ ID NOs: 119, 122, 125, 128, 131, 134, 137, 140, and 143, LCDR2 comprises and amino acid sequence selected from the group consisting of SEQ ID NOs: 120, 123, 126, 129, 132, 135, 138, 141, and 144, and LCDR3 comprises an amino acid sequence selected from the group consisting of SEQ ID NOs: 121, 124, 127, 130, 133, 136, 139, 142, and 145;
ii. a CD8 transmembrane domain;
iii. a 4-1BB costimulatory domain; and
iv. a CD3ζ intracellular signaling domain.

Embodiment 26 provides a chimeric antigen receptor (CAR) comprising:
i. an antigen binding domain comprising: a heavy chain variable region comprising any of the amino acid sequences set forth in SEQ ID NOs: 146-155; and a light chain variable region comprising any of the amino acid sequences set forth in SEQ ID NOs: 156-165;
ii. a CD8 transmembrane domain;
iii. a 4-1BB costimulatory domain; and
iv. a CD3ζ intracellular signaling domain.

Embodiment 27 provides a chimeric antigen receptor (CAR) comprising any one of the amino acid sequences set forth in SEQ ID NOs: 175, 177, 179, 181, 183, 185, 187, 189, 191, 193, 195, 197, 199, 201, 203, 205, 207, 209, and 211.

Embodiment 28 provides a chimeric antigen receptor (CAR) consisting of any one of the amino acid sequences set forth in SEQ ID NOs: 175, 177, 179, 181, 183, 185, 187, 189, 191, 193, 195, 197, 199, 201, 203, 205, 207, 209, and 211.

Embodiment 29 provides a nucleic acid encoding the CAR of any one of embodiments 1-28.

Embodiment 30 provides a nucleic acid encoding a chimeric antigen receptor (CAR) comprising an antigen binding domain, a transmembrane domain, and an intracellular domain, wherein the antigen binding domain comprises:
i. a heavy chain variable region that comprises three heavy chain complementarity determining regions (HCDRs), wherein HCDR1 comprises an amino acid sequence selected from the group consisting of SEQ ID NOs: 89, 92, 95, 98, 101, 104, 107, 110, 113, and 116, HCDDR2 comprises an amino acid sequence selected from the group consisting of SEQ ID NOs: 90, 93, 96, 99, 102, 105, 108, 111, 114, and 117, and HCDR3 comprises and amino acid sequence selected from the group comprising SEQ ID NOs: 91, 94, 97, 100, 103, 106, 109, 112, 115, and 118; and
ii. a light chain variable region that comprises three light chain complementarity determining regions (LCDRs), wherein LCDR1 comprises an amino acid sequence selected from the group consisting of SEQ ID NOs: 119, 122, 125, 128, 131, 134, 137, 140, and 143, LCDR2 comprises and amino acid sequence selected from the group consisting of SEQ ID NOs: 120, 123, 126, 129, 132, 135, 138, 141, and 144, and LCDR3 comprises an amino acid sequence selected from the group consisting of SEQ ID NOs: 121, 124, 127, 130, 133, 136, 139, 142, and 145.

Embodiment 31 provides the nucleic acid of embodiment 30, wherein the CAR binds CD45.

Embodiment 32 provides the nucleic acid of embodiment 30 or 31, wherein the antigen binding domain comprises an antibody or an antigen-binding fragment thereof.

Embodiment 33 provides the nucleic acid of embodiment 32, wherein the antigen-binding fragment is selected from the group consisting of a Fab, a single-chain variable fragment (scFv), or a single-domain antibody.

Embodiment 34 provides the nucleic acid of embodiment 33, wherein the antibody is a scFv.

Embodiment 35 provides a nucleic acid encoding a chimeric antigen receptor (CAR) comprising an antigen binding domain, a transmembrane domain, and an intracellular signaling domain, wherein the antigen binding domain comprises:
i. a heavy chain variable region comprising a nucleic acid encoding any of the amino acid sequences set forth in SEQ ID NOs: 146-155; and
ii. a light chain variable region comprising a nucleic acid encoding any of the amino acid sequences set forth in SEQ ID NOs: 156-165.

Embodiment 36 provides a nucleic acid encoding a chimeric antigen receptor (CAR) comprising an antigen binding domain, a transmembrane domain, and an intracellular signaling domain, wherein the antigen binding domain is encoded by any one of the nucleotide sequences set forth in SEQ ID NOs: 174, 176, 178, 180, 182, 184, 186, 188, 190, 192, 194, 196, 198, 200, 202, 204, 208, and 210.

Embodiment 37 provides the nucleic acid of any one of embodiments 29-36, wherein the transmembrane domain is selected from the group consisting of an artificial hydrophobic sequence, and a transmembrane domain of a type I transmembrane protein, an alpha, beta, or zeta chain of a T cell receptor, CD28, CD3epsilon, CD45, CD4, CD5, CD8, CD9, CD16, CD22, CD33, CD37, CD64, CD80, CD86, OX40 (CD134), 4-1BB (CD137), and CD154.

Embodiment 38 provides the nucleic acid of Embodiment 37, wherein the transmembrane domain comprises a transmembrane domain of CD8.

Embodiment 39 provides the nucleic acid of any one of embodiments 29-36, wherein the CAR further comprises a hinge domain.

Embodiment 40 provides the nucleic acid of embodiment 39, wherein the hinge domain is selected from the group consisting of an Fc fragment of an antibody, a hinge region of an antibody, a CH2 region of an antibody, a CH3 region of an antibody, an artificial hinge domain, a hinge comprising an amino acid sequence of a CD8 hinge, or any combination thereof.

Embodiment 41 provides the nucleic acid of any one of embodiments 39 or 40, wherein the artificial hinge domain is a CD8 hinge.

Embodiment 42 provides the nucleic acid of any one of embodiments 29-36, wherein the intracellular domain comprises a costimulatory signaling domain and an intracellular signaling domain.

Embodiment 43 provides the nucleic acid of embodiment 42, wherein the costimulatory signaling domain comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFI-II, Fas, CD30, CD40, ICOS, NKG2C, and B7-H3 (CD276), or a variant thereof.

Embodiment 44 provides the nucleic acid of embodiment 42 or 43, wherein the costimulatory signaling domain comprises a costimulatory domain of 4-1BB.

Embodiment 45 provides the nucleic acid of any one of embodiment 29-36, wherein the intracellular signaling domain comprises an intracellular domain selected from the group consisting of cytoplasmic signaling domains of a human CD3 zeta chain (CD3ζ), FcyRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

Embodiment 46 provides the nucleic acid of embodiment 45, wherein the intracellular signaling domain comprises an intracellular domain of CD3ζ, Embodiment 47 provides a vector comprising the nucleic acid of any one of embodiments 29-46.

Embodiment 48 provides the vector of embodiment 47, wherein the vector is an expression vector.

Embodiment 49 provides the vector of embodiment 47, wherein the vector further comprising a CRISPR-based gene editing system.

Embodiment 50 provides the vector of embodiment 49, wherein the CRISPR-based gene editing system downregulates the expression of endogenous CD45.

Embodiment 51 provides the vector of embodiment 49, wherein the CRISPR-based gene editing system alters the epitope of CD45 recognized by the CAR.

Embodiment 52 provides the vector of embodiment 51, wherein the alteration of the epitope of CD45 renders it unable to be bound by the CAR.

Embodiment 53 provides the vector of embodiment 51, wherein the alteration is selected from the group consisting of a substitution, an insertion, a deletion, and an insertion/deletion.

Embodiment 54 provides the vector of embodiment 53, wherein the alteration is mediated by a CRISPR system comprising a CRISPR nuclease and a guide RNA.

Embodiment 55 provides the vector of embodiment 54, wherein the CRISPR nuclease is CRISPR/Cas9.

Embodiment 56 provides the vector of embodiment 54, wherein the guide RNA comprises a guide sequence that is sufficiently complementary with a target sequence in the endogenous gene locus encoding CD45.

Embodiment 57 provides the vector of any one of embodiments 54-56, wherein the guide RNA comprises a nucleic acid sequence set forth in any one of SEQ ID NOs: 212-223.

Embodiment 58 provides the vector of embodiment 54, wherein the modification is mediated by a CRISPR system comprising a base editor system.

Embodiment 59 provides the vector of embodiment 58, wherein the base editor system comprises a single guide RNA.

Embodiment 60 provides the vector of embodiment 59, wherein the single guide RNA comprises a nucleic acid sequence set forth in any one of SEQ ID NOs: 224-252.

Embodiment 61 provides the vector of embodiment 58, wherein the base editor system comprises a nucleic acid sequence set forth in any one of SEQ ID NOs: 253-258.

Embodiment 62 provides a cell comprising the CAR of any one of embodiments 1-28, the nucleic acid of any one of embodiments 29-46, or the vector of any one of embodiments 47-61.

Embodiment 63 provides the cell of embodiment 62, wherein the cell is selected from the group consisting of a T cell, a natural killer (NK) cell, a cytotoxic T lymphocyte (CTL), and a regulatory T cell.

Embodiment 64 provides a modified immune cell or precursor cell thereof, comprising a chimeric antigen receptor (CAR) comprising an antigen binding domain, a transmembrane domain, and an intracellular domain, wherein the antigen binding domain comprises:
i. a heavy chain variable region that comprises three heavy chain complementarity determining regions (HCDRs), wherein HCDR1 comprises an amino acid sequence selected from the group consisting of SEQ ID NOs: 89, 92, 95, 98, 101, 104, 107, 110, 113, and 116, HCDDR2 comprises an amino acid sequence selected from the group consisting of SEQ ID NOs: 90, 93, 96, 99, 102, 105, 108, 111, 114, and 117, and HCDR3 comprises and amino acid sequence selected from the group comprising SEQ ID NOs: 91, 94, 97, 100, 103, 106, 109, 112, 115, and 118;
ii. a light chain variable region that comprises three light chain complementarity determining regions (LCDRs), wherein LCDR1 comprises an amino acid sequence selected from the group consisting of SEQ ID NOs: 119, 122, 125, 128, 131, 134, 137, 140, and 143, LCDR2 comprises and amino acid sequence selected from the group consisting of SEQ ID NOs: 120, 123, 126, 129, 132, 135, 138, 141, and 144, and LCDR3 comprises an amino acid sequence selected from the group consisting of SEQ ID NOs: 121, 124, 127, 130, 133, 136, 139, 142, and 145.
iii. a CRISPR-mediated modification in an endogenous gene locus encoding CD45.

Embodiment 65 provides the modified immune cell of embodiment 64, wherein the CAR binds CD45.

Embodiment 66 provides the modified immune cell of embodiment 64, wherein the CRISPR-mediated modification is a deletion that downregulates the expression of endogenous CD45.

Embodiment 67 provides the modified immune cell of embodiment 64, wherein the CRISPR-mediated modification alters the epitope of CD45 recognized by the CAR.

Embodiment 68 provides the modified immune cell of embodiment 67, wherein the alteration of the epitope of CD45 renders it unable to be bound by the CAR.

Embodiment 69 provides the modified immune cell of embodiment 64, wherein the modification is selected from the group consisting of a substitution, an insertion, a deletion, and an insertion/deletion.

Embodiment 70 provides the modified immune cell of embodiment 64, wherein the modification is mediated by a CRISPR system comprising a CRISPR nuclease and a guide RNA.

Embodiment 71 provides the modified immune cell of embodiment 70, wherein the CRISPR nuclease is CRISPR/Cas9.

Embodiment 72 provides the modified immune cell of embodiment 70, wherein the guide RNA comprises a guide sequence that is sufficiently complementary with a target sequence in the endogenous gene locus encoding CD45.

Embodiment 73 provides the modified immune cell of any one of embodiment 70-72, wherein the guide RNA comprises a nucleic acid sequence set forth in any one of SEQ ID NOs: 212-223.

Embodiment 74 provides the modified immune cell of embodiment 64, wherein the modification is mediated by a CRISPR system comprising a base editor system.

Embodiment 75 provides the modified immune cell of embodiment 74, wherein the base editor system comprises a single guide RNA.

Embodiment 76 provides the modified immune cell of 75, wherein the single guide RNA comprises a nucleic acid sequence set forth in any one of SEQ ID NOs: 224-252.

Embodiment 77 provides the modified immune cell of embodiment 74, wherein the base editor system comprises a nucleic acid sequence set forth in any one of SEQ ID NOs: 253-258.

Embodiment 78 provides the modified immune cell of embodiment 64, wherein the modified endogenous gene locus of CD45 comprises a nucleotide sequence set forth in any one of SEQ ID NOs: 3, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, and 87.

Embodiment 79 provides the modified immune cell of embodiment 64, wherein the modified endogenous gene locus of CD45 encodes a polypeptide comprising the amino acid sequence set forth in any one of SEQ ID NOs: 4, 8, 10, 12, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, and 88.

Embodiment 80 provides the modified immune cell of embodiment 64, wherein the CAR comprises an antigen binding domain selected from the group consisting of an antibody, an scFv, and a Fab.

Embodiment 81 provides the modified immune cell of embodiment 64, wherein the CAR further comprises a hinge domain.

Embodiment 82 provides the modified immune cell of embodiment 81 wherein the hinge domain selected from the group consisting of an Fc fragment of an antibody, a hinge region of an antibody, a CH2 region of an antibody, a CH3 region of an antibody, an artificial hinge domain, a hinge comprising an amino acid sequence of CD8, or any combination thereof.

Embodiment 83 provides the modified immune cell of embodiment 64, wherein the CAR comprises a transmembrane domain selected from the group consisting of an artificial hydrophobic sequence and transmembrane domain of a type I transmembrane protein, an alpha, beta, or zeta chain of a T cell receptor, CD28, CD3 epsilon, CD45, CD4, CD5, CD8, CD9, CD16, CD22, CD33, CD37, CD64, CD80, CD86, CD134, CD137, and CD154.

Embodiment 84 provides the modified immune cell of embodiment 64, wherein the CAR comprises at least one co-stimulatory domain selected from the group consisting of co-stimulatory domains of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3.

Embodiment 85 provides the modified immune cell of embodiment 64, wherein the CAR comprises an intracellular domain comprising an intracellular domain selected from the group consisting of cytoplasmic signaling domains of a human CD3 zeta chain, FcγRIII, FcsRI, a cytoplasmic tail of an Fc receptor, an immunoreceptor tyrosine-based activation motif (ITAM) bearing cytoplasmic receptors, TCR zeta, FcR gamma, FcR beta, CD3 gamma, CD3 delta, CD3 epsilon, CD5, CD22, CD79a, CD79b, and CD66d.

Embodiment 86 provides the modified immune cell of embodiment 64, wherein the modified cell is resistant to CAR T cell fratricide.

Embodiment 87 provides the modified immune cell of embodiment 64, wherein the modified cell is an autologous cell.

Embodiment 88 provides the modified immune cell of embodiment 64, wherein the modified cell is a cell isolated from a human subject.

Embodiment 89 provides the modified immune cell of embodiment 64, wherein the modified cell is a modified T cell.

Embodiment 90 provides a method for generating a modified immune cell or precursor cell thereof, comprising:
i. introducing into an immune or precursor cell a CRISPR system comprising one or more polypeptides and/or nucleic acids capable of modifying expression of endogenous CD45 gene; and
ii. introducing into the immune or precursor cell a nucleic acid encoding a chimeric antigen receptor (CAR), wherein the CAR has affinity for an antigen on a target cell.

Embodiment 91 provides the method of embodiment 90, wherein the one or more polypeptides and/or nucleic acids capable of downregulating gene expression of endogenous CD45 introduces a CRISPR-mediated modification in an endogenous gene locus encoding CD45.

Embodiment 92 provides the method of embodiment 91, wherein the modification is selected from the group consisting of a substitution, an insertion, a deletion, and an insertion/deletion.

Embodiment 93 provides the method of embodiment 90, wherein the CRISPR system comprises a CRISPR nuclease and a guide RNA.

Embodiment 94 provides the method of embodiment 93, wherein the CRISPR nuclease is Cas9.

Embodiment 95 provides the method of embodiment 93, wherein the guide RNA comprises a guide sequence that is sufficiently complementary with a target sequence in the endogenous gene locus encoding CD45.

Embodiment 96 provides the method of embodiment 93, wherein the guide RNA comprises a nucleic acid sequence set forth in any one of SEQ ID NO: 212-223.

Embodiment 97 provides the method of embodiment 90, wherein the CRISPR system comprises a base editor system.

Embodiment 98 provides the method of embodiment 97, wherein the base editor system comprises a single guide RNA.

Embodiment 99 provides the method of embodiment 98, wherein the single guide RNA comprises a nucleic acid sequence set forth in any one of SEQ ID NOs: 224-252.

Embodiment 100 provides the method of embodiment 97, wherein the base editor system comprises a nucleic acid sequence set forth in any one of SEQ ID NOs: 253-258.

Embodiment 101 provides a method of treating cancer in a subject in need thereof, comprising:
i. administering to the subject a modified immune cell of embodiments 51-76, or a modified immune or precursor cell generated by the method of embodiments 77-87; and
ii. administering to the subject a modified precursor cell comprising a CRISPR-mediated modification in an endogenous gene locus encoding CD45.

Embodiment 102 provides the method of embodiment 101, wherein the modified precursor cell is selected from the group consisting of a bone marrow stem cell, a hematopoietic progenitor cell, and a cord blood stem cell.

Embodiment 103 provides the method of embodiment 101, wherein the subject is conditioned by pre-treatment with radiation.

Embodiment 104 provides the method of embodiment 101, wherein the subject conditioned by pre-treatment with a chemotherapy.

Embodiment 105 provides the method of embodiment 101, wherein the subject is conditioned by pre-treatment with radiation and chemotherapy.

Embodiment 106 provides the method of any one of embodiments 104 or 105, wherein the chemotherapy is selected from the group consisting of anti-thymocyte globulin, carmustine, bulsulfan, carboplatin, cyclosporin A, cyclophosphamide, etoposide, fludarabine, melphalan, methotrexate, tacrolimus, thiotepa, topotecan, or any combination thereof.

Embodiment 107 provides the method of embodiment 101, wherein the cancer is a hematologic cancer.

Embodiment 108 provides the method of embodiment 107, wherein the hematologic cancer is selected from the group consisting of a B-cell lymphoma, a B-cell leukemia, a multiple myeloma, and an acute myeloid leukemia.

Embodiment 109 provides the method of embodiment 101, wherein the modified precursor cell is administered first.

Embodiment 110 provides the method of embodiment 101, wherein the modified precursor cell and modified immune cell are administered concurrently.

Embodiment 111 provides a method of conditioning a subject prior to bone marrow transplant, comprising administering to the subject an effective amount of a modified T cell comprising a modified immune cell of embodiments 51-76, or a modified immune or precursor cell generated by the method of embodiments 77-87.

Embodiment 112 provides the method of embodiment 111, wherein conditioning further comprises administering an effective amount of a chemotherapy, radiation, or a combination thereof.

Embodiment 113 provides the method of embodiment 112, wherein the chemotherapy, radiation, or combination thereof is administered prior to administration of the modified T cell or modified immune cell.

Embodiment 114 provides the method of embodiment 113, wherein the chemotherapy is selected from the group consisting of anti-thymocyte globulin, carmustine, bulsulfan, carboplatin, cyclosporin A, cyclophosphamide, etoposide, fludarabine, melphalan, methotrexate, tacrolimus, thiotepa, topotecan, or any combination thereof.

Embodiment 115 provides a method of treating an HIV infection in a subject, comprising:
i. administering to the subject a modified immune cell of embodiments 64-89, or a modified immune or precursor cell generated by the method of embodiments 90-100; and
ii. administering to the subject a modified precursor cell comprising a CRISPR-mediated modification in a first endogenous gene locus and one or more second endogenous gene loci, thereby treating the HIV infection.

Embodiment 116 provides the method of embodiment 115, wherein the first endogenous gene locus is CD45.

Embodiment 117 provides the method of embodiment 115, wherein the one or more second endogenous gene loci are selected from the group consisting of CD4, CCR5, CXCR4, and any combination thereof.

Embodiment 118 provides the method of embodiment 115, wherein the CRISPR-mediated modification is selected from the group consisting of a substitution, an insertion, a deletion, and an insertion/deletion.

Embodiment 119 provides the method of embodiment 115, wherein the CRISPR-mediated modification of the one or more second endogenous gene loci reduces or eliminates the binding of the protein encoded by the loci with HIV gp120.

Embodiment 120 provides the method of embodiment 115, wherein the modified precursor cell is selected from the group consisting of a bone marrow stem cell, a hematopoietic progenitor cell, and a cord blood stem cell.

Embodiment 121 provides the method of embodiment 101, wherein the subject is conditioned by pre-treatment with radiation.

Embodiment 122 provides the method of embodiment 101, wherein the subject conditioned by pre-treatment with a chemotherapy.

Embodiment 123 provides the method of embodiment 101, wherein the subject is conditioned by pre-treatment with radiation and chemotherapy.

Embodiment 124 provides the method of any one of embodiments 104 or 105, wherein the chemotherapy is selected from the group consisting of anti-thymocyte globulin, carmustine, bulsulfan, carboplatin, cyclosporin A, clofarabine, cyclophosphamide, etoposide, fludarabine, melphalan, methotrexate, tacrolimus, thiotepa, topotecan, or any combination thereof.

Embodiment 125 provides a method of treating a hematologic malignancy in a subject in need thereof, comprising:
i. administering to the subject a CD33-targeted therapy comprising a CD33-specific CAR-T cell; and
ii. administering to the subject a population of modified precursor cells comprising a modification in an endogenous gene locus such that the precursor cells are resistant to the CD33-targeted therapy.

Embodiment 126 provides the method of embodiment 125, wherein the endogenous gene locus is CD33.

Embodiment 127 provides the method of embodiment 125, wherein the modification is a CRISPR-mediated modification.

Embodiment 128 provides the method of embodiment 125, wherein the CRISPR-mediated modification is selected from the group consisting of a substitution, an insertion, a deletion, and an insertion/deletion.

Embodiment 129 provides the method of embodiment 125, wherein the modified precursor cell is selected from the group consisting of a bone marrow stem cell, a hematopoietic progenitor cell, and a cord blood stem cell.

Embodiment 130 provides the method of embodiment 125, wherein the subject is conditioned by pre-treatment with radiation.

Embodiment 131 provides the method of embodiment 125, wherein the subject conditioned by pre-treatment with a chemotherapy.

Embodiment 132 provides the method of embodiment 131, wherein the subject is conditioned by pre-treatment with radiation and chemotherapy.

Embodiment 133 provides the method of any one of embodiments 131 or 132, wherein the chemotherapy is selected from the group consisting of anti-thymocyte globulin, carmustine, bulsulfan, carboplatin, cyclosporin A, clofarabine, cyclophosphamide, etoposide, fludarabine, melphalan, methotrexate, tacrolimus, thiotepa, topotecan, or any combination thereof.

Embodiment 134 provides the method of embodiment 125, wherein the hematologic malignancy is a myeloid malignancy selected from the group consisting of acute myelogenous leukemia (AML), chronic myelogenous leukemia (CML), myelodysplastic neoplasm, and myeloproliferative neoplasm.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. A chimeric antigen receptor (CAR) comprising an antigen binding domain, a transmembrane domain, and an intracellular domain, wherein the antigen binding domain comprises:
i. a heavy chain variable region that comprises three heavy chain complementarity determining regions (HCDRs), wherein HCDR1 comprises an amino acid sequence selected from the group consisting of SEQ ID NOs: 89, 92, 95, 98, 101, 104, 107, 110, 113, and 116, HCDDR2 comprises an amino acid sequence selected from the group consisting of SEQ ID NOs: 90, 93, 96, 99, 102, 105, 108, 111, 114, and 117, and HCDR3 comprises and amino acid sequence selected from the group comprising SEQ ID NOs: 91, 94, 97, 100, 103, 106, 109, 112, 115, and 118; and
ii. a light chain variable region that comprises three light chain complementarity determining regions (LCDRs), wherein LCDR1 comprises an amino acid sequence selected from the group consisting of SEQ ID NOs: 119, 122, 125, 128, 131, 134, 137, 140, and 143, LCDR2 comprises and amino acid sequence selected from the group consisting of SEQ ID NOs: 120, 123, 126, 129, 132, 135, 138, 141, and 144, and LCDR3 comprises an amino acid sequence selected from the group consisting of SEQ ID NOs: 121, 124, 127, 130, 133, 136, 139, 142, and 145.

2. The CAR of claim 1, wherein the CAR binds CD45.

3. The CAR of claim 1, wherein the antigen binding domain comprises an antibody or an antigen-binding fragment thereof.

4. The CAR of claim 3, wherein the antigen-binding fragment is selected from the group consisting of a Fab, a single-chain variable fragment (scFv), or a single-domain antibody.

5. The CAR of claim 4, wherein the antigen-binding fragment is a scFv.

6. The CAR of claim 1, wherein the antigen binding domain comprises a heavy chain variable region comprising an amino acid sequence having at least 95%-99% identity to the amino acid sequence of any of the heavy chain variable regions set forth in SEQ ID NOs: 146-155.

7. The CAR of claim 1, wherein the antigen binding domain comprises a heavy chain variable region comprising an amino acid sequence of any of the heavy chain variable regions set forth in SEQ ID NOs: 146-155.

8. The CAR of claim 1, wherein the antigen binding domain comprises a heavy chain variable region consisting of an amino acid sequence of any of the heavy chain variable regions set forth in SEQ ID NOs: 146-155.

9. The CAR of claim 1, wherein the antigen binding domain comprises a light chain variable region comprising an amino acid sequence having at least 95-99% identity to the amino acid sequence of any of the light chain variable regions set forth in SEQ ID NOs: 156-165.

10. The CAR of claim 1, wherein the antigen binding domain comprises a light chain variable region comprising an amino acid sequence of any of the light chain variable regions set forth in SEQ ID NOs: 156-165.

11. The CAR of claim 1, wherein the antigen binding domain comprises a light chain variable region consisting of an amino acid sequence of any of the light chain variable regions set forth in SEQ ID NOs: 156-165.

12. A chimeric antigen receptor (CAR) comprising an antigen binding domain, a transmembrane domain, and an intracellular domain, wherein the antigen binding domain comprises a heavy chain variable region comprising any of the amino acid sequences set forth in SEQ ID NO: 146-155; and a light chain variable region comprising any of the amino acid sequences set forth in SEQ ID NO: 156-165.

13. A chimeric antigen receptor (CAR) comprising an antigen binding domain, a transmembrane domain, and an intracellular signaling domain, wherein the antigen binding domain comprises any one of the amino acid sequences set forth in SEQ ID NOs: 175, 177, 179, 181, 183, 185, 187, 189, 191, 193, 195, 197, 199, 201, 203, 205, 207, 209, and 211.

14. The CAR of claim 1, wherein the transmembrane domain is selected from the group consisting of an artificial hydrophobic sequence, and a transmembrane domain of a type I transmembrane protein, an alpha, beta, or zeta chain of a T cell receptor, CD28, CD3 epsilon, CD45, CD4, CD5, CD8, CD9, CD16, CD22, CD33, CD37, CD64, CD80, CD86, OX40 (CD134), 4-1BB (CD137), and CD154.

15. The CAR of claim 1, wherein the transmembrane domain comprises a transmembrane domain of CD8 comprising an amino acid sequence set forth in SEQ ID NO: 169.

16. The CAR of claim 1, further comprising a hinge domain.

17. The CAR of claim 16, wherein the hinge domain is selected from the group consisting of an Fc fragment of an antibody, a hinge region of an antibody, a CH2 region of an antibody, a CH3 region of an antibody, an artificial hinge domain, a hinge comprising an amino acid sequence of a CD8 hinge, or any combination thereof.

18. The CAR of claim 17, wherein the hinge domain is a CD8 hinge comprising an amino acid sequence set forth in SEQ ID NO: 168.

19. The CAR of claim 1, wherein the intracellular domain comprises a costimulatory signaling domain and an intracellular signaling domain.

20. The CAR of claim 19, wherein the costimulatory signaling domain comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFI-II, Fas, CD30, CD40, ICOS, NKG2C, and B7-H3 (CD276), or a variant thereof.

21. The CAR of claim 19, wherein the costimulatory signaling domain comprises a costimulatory domain of 4-1BB comprising an amino acid sequence set forth in SEQ ID NO: 170.

22. The CAR of claim 12, wherein the intracellular signaling domain comprises an intracellular domain selected from the group consisting of cytoplasmic signaling domains of a human CD3 zeta chain (CD3ζ), FcyRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

23. The CAR of claim 19, wherein the intracellular signaling domain comprises an intracellular domain of CD3ζ comprising an amino acid sequence set forth in SEQ ID NO: 171.

24. (canceled)

25. A chimeric antigen receptor (CAR) comprising:
i. an antigen binding domain comprising: a heavy chain variable region that comprises three heavy chain complementarity determining regions (HCDRs), wherein HCDR1 comprises an amino acid sequence selected from the group consisting of SEQ ID NOs: 89, 92, 95, 98, 101, 104, 107, 110, 113, and 116, HCDDR2 comprises an amino acid sequence selected from the group consisting of SEQ ID NOs: 90, 93, 96, 99, 102, 105, 108, 111, 114, and 117, and HCDR3 comprises and amino acid sequence selected from the group comprising SEQ ID NOs: 91, 94, 97, 100, 103, 106, 109, 112, 115, and 118; and a light chain variable region that comprises three light chain complementarity determining regions (LCDRs), wherein LCDR1 comprises an amino acid sequence selected from the group consisting of SEQ ID NOs: 119, 122, 125, 128, 131, 134, 137, 140, and 143, LCDR2 comprises and amino acid sequence selected from the group consisting of SEQ ID NOs: 120, 123, 126, 129, 132, 135, 138, 141, and 144, and LCDR3 comprises an amino acid sequence selected from the group consisting of SEQ ID NOs: 121, 124, 127, 130, 133, 136, 139, 142, and 145;
ii. a CD8 transmembrane domain;
iii. a 4-1BB costimulatory domain; and
iv. a CD3ζ intracellular signaling domain.

26. A chimeric antigen receptor (CAR) comprising:
i. an antigen binding domain comprising: a heavy chain variable region comprising any of the amino acid sequences set forth in SEQ ID NOs: 146-155;
and a light chain variable region comprising any of the amino acid sequences set forth in SEQ ID NOs: 156-165;
ii. a CD8 transmembrane domain;
iii. a 4-1BB costimulatory domain; and
iv. a CD3ζ intracellular signaling domain.

27. A chimeric antigen receptor (CAR) comprising any one of the amino acid sequences set forth in SEQ ID NOs: 175, 177, 179, 181, 183, 185, 187, 189, 191, 193, 195, 197, 199, 201, 203, 205, 207, 209, and 211.

28. A chimeric antigen receptor (CAR) consisting of any one of the amino acid sequences set forth in SEQ ID NOs: 175, 177, 179, 181, 183, 185, 187, 189, 191, 193, 195, 197, 199, 201, 203, 205, 207, 209, and 211.

29. A nucleic acid encoding the CAR of claim 1.

30. A nucleic acid encoding a chimeric antigen receptor (CAR) comprising an antigen binding domain, a transmembrane domain, and an intracellular domain, wherein the antigen binding domain comprises:
i. a heavy chain variable region that comprises three heavy chain complementarity determining regions (HCDRs), wherein HCDR1 comprises an amino acid sequence selected from the group consisting of SEQ ID NOs: 89, 92, 95, 98, 101, 104, 107, 110, 113, and 116, HCDDR2 comprises an amino acid sequence selected from the group consisting of SEQ ID NOs: 90, 93, 96, 99, 102, 105, 108, 111, 114, and 117, and HCDR3 comprises and amino acid sequence selected from the group comprising SEQ ID NOs: 91, 94, 97, 100, 103, 106, 109, 112, 115, and 118; and
ii. a light chain variable region that comprises three light chain complementarity determining regions (LCDRs), wherein LCDR1 comprises an amino acid sequence selected from the group consisting of SEQ ID NOs: 119, 122, 125, 128, 131, 134, 137, 140, and 143, LCDR2 comprises and amino acid sequence selected from the group consisting of SEQ ID NOs: 120, 123, 126, 129, 132, 135, 138, 141, and 144, and LCDR3 comprises an amino acid sequence selected from the group consisting of SEQ ID NOs: 121, 124, 127, 130, 133, 136, 139, 142, and 145.

31. The nucleic acid of claim 30, wherein the CAR binds CD45.

32. The nucleic acid of claim 30, wherein the antigen binding domain comprises an antibody or an antigen-binding fragment thereof.

33. The nucleic acid of claim 32, wherein the antigen-binding fragment is selected from the group consisting of a Fab, a single-chain variable fragment (scFv), or a single-domain antibody.

34. The nucleic acid of claim 32, wherein the antibody is a scFv.

35. A nucleic acid encoding a chimeric antigen receptor (CAR) comprising an antigen binding domain, a transmembrane domain, and an intracellular signaling domain, wherein the antigen binding domain comprises:
i. a heavy chain variable region comprising a nucleic acid encoding any of the amino acid sequences set forth in SEQ ID NOs: 146-155; and
ii. a light chain variable region comprising a nucleic acid encoding any of the amino acid sequences set forth in SEQ ID NOs: 156-165.

36. A nucleic acid encoding a chimeric antigen receptor (CAR) comprising an antigen binding domain, a transmembrane domain, and an intracellular signaling domain, wherein the antigen binding domain is encoded by any one of the nucleotide sequences set forth in SEQ ID NOs: 174, 176, 178, 180, 182, 184, 186, 188, 190, 192, 194, 196, 198, 200, 202, 204, 208, and 210.

37. The nucleic acid of claim 30, wherein the transmembrane domain is selected from the group consisting of an artificial hydrophobic sequence, and a transmembrane domain of a type I transmembrane protein, an alpha, beta, or zeta chain of a T cell receptor, CD28, CD3epsilon, CD45, CD4, CD5, CD8, CD9, CD16, CD22, CD33, CD37, CD64, CD80, CD86, OX40 (CD134), 4-1BB (CD137), and CD154.

38. The nucleic acid of claim 30, wherein the transmembrane domain comprises a transmembrane domain of CD8.

39. The nucleic acid of claim 30, wherein the CAR further comprises a hinge domain.

40. The nucleic acid of claim 39, wherein the hinge domain is selected from the group consisting of an Fc fragment of an antibody, a hinge region of an antibody, a CH2 region of an antibody, a CH3 region of an antibody, an artificial hinge domain, a hinge comprising an amino acid sequence of a CD8 hinge, or any combination thereof.

41. The nucleic acid of claim 39, wherein the artificial hinge domain is a CD8 hinge.

42. The nucleic acid of claim 30, wherein the intracellular domain comprises a costimulatory signaling domain and an intracellular signaling domain.

43. The nucleic acid of claim 42, wherein the costimulatory signaling domain comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFI-II, Fas, CD30, CD40, ICOS, NKG2C, and B7-H3 (CD276), or a variant thereof.

44. The nucleic acid of claim 42, wherein the costimulatory signaling domain comprises a costimulatory domain of 4-1BB.

45. The nucleic acid of claim 42, wherein the intracellular signaling domain comprises an intracellular domain selected from the group consisting of cytoplasmic signaling domains of a human CD3 zeta chain (CD3ζ), FcγRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

46. The nucleic acid of claim 42, wherein the intracellular signaling domain comprises an intracellular domain of CD3ζ,

47. A vector comprising the nucleic acid of claim 30.

48. The vector of claim 47, wherein the vector is an expression vector.

49. The vector of claim 47, wherein the vector further comprising a CRISPR-based gene editing system.

50. The vector of claim 49, wherein the CRISPR-based gene editing system downregulates the expression of endogenous CD45.

51. The vector of claim 49, wherein the CRISPR-based gene editing system alters the epitope of CD45 recognized by the CAR, and comprises a CRISPR nuclease and a guide RNA.

52. The vector of claim 51, wherein the alteration of the epitope of CD45 renders it unable to be bound by the CAR.

53. The vector of claim 51, wherein the alteration is selected from the group consisting of a substitution, an insertion, a deletion, and an insertion/deletion.

54. (canceled)

55. The vector of claim 51, wherein the CRISPR nuclease is CRISPR/Cas9.

56. The vector of claim 51, wherein the guide RNA comprises a guide sequence that is sufficiently complementary with a target sequence in the endogenous gene locus encoding CD45.

57. The vector of claim 51, wherein the guide RNA comprises a nucleic acid sequence set forth in any one of SEQ ID NOs: 212-223.

58. The vector of claim 51, wherein the CRISPR system is a base editor system.

59. The vector of claim 58, wherein the base editor system comprises a single guide RNA.

60. The vector of claim 59, wherein the single guide RNA comprises a nucleic acid sequence set forth in any one of SEQ ID NOs: 224-252.

61. The vector of claim 58, wherein the base editor system comprises a nucleic acid sequence set forth in any one of SEQ ID NOs: 253-258.

62. (canceled)

63. (canceled)

64. A modified immune cell or precursor cell thereof, comprising a CRISPR-mediated modification in an endo endogenous gene locus encoding CD45, and a chimeric antigen receptor (CAR) comprising an antigen binding domain, a transmembrane domain, and an intracellular domain, wherein the antigen binding domain comprises:
i. a heavy chain variable region that comprises three heavy chain complementarity determining regions (HCDRs), wherein HCDR1 comprises an amino acid sequence selected from the group consisting of SEQ ID NOs: 89, 92, 95, 98, 101, 104, 107, 110, 113, and 116, HCDDR2 comprises an amino acid sequence selected from the group consisting of SEQ ID NOs: 90, 93, 96, 99, 102, 105, 108, 111, 114, and 117, and HCDR3 comprises and amino acid sequence selected from the group comprising SEQ ID NOs: 91, 94, 97, 100, 103, 106, 109, 112, 115, and 118; and
ii. a light chain variable region that comprises three light chain complementarity determining regions (LCDRs), wherein LCDR1 comprises an amino acid sequence selected from the group consisting of SEQ ID NOs: 119, 122, 125, 128, 131, 134, 137, 140, and 143, LCDR2 comprises and amino acid sequence selected from the group consisting of SEQ ID NOs: 120, 123, 126, 129, 132, 135, 138, 141, and 144, and LCDR3 comprises an amino acid sequence selected from the group consisting of SEQ ID NOs: 121, 124, 127, 130, 133, 136, 139, 142, and 145,
wherein the CRISPR-mediated modification is mediated by a CRISPR system comprising a CRISPR nuclease and a guide RNA.

65. The modified immune cell of claim 64, wherein the CAR binds CD45.

66. The modified immune cell of claim 64, wherein the CRISPR-mediated modification is a deletion that downregulates the expression of endogenous CD45.

67. The modified immune cell of claim 64, wherein the CRISPR-mediated modification alters the epitope of CD45 recognized by the CAR.

68. The modified immune cell of claim 67, wherein the alteration of the epitope of CD45 renders it unable to be bound by the CAR.

69. The modified immune cell of claim 64, wherein the modification is selected from the group consisting of a substitution, an insertion, a deletion, and an insertion/deletion.

70. (canceled)

71. The modified immune cell of claim 64, wherein the CRISPR nuclease is CRISPR/Cas9.

72. The modified immune cell of claim 64, wherein the guide RNA comprises a guide sequence that is sufficiently complementary with a target sequence in the endogenous gene locus encoding CD45.

73. The modified immune cell of claim 64, wherein the guide RNA comprises a nucleic acid sequence set forth in any one of SEQ ID NOs: 212-223.

74. The modified immune cell of claim 64, wherein the CRISPR system comprises a base editor system.

75. The modified immune cell of claim 74, wherein the base editor system comprises a single guide RNA comprising a nucleic acid sequence set forth in any one of SEQ ID NOs: 224-252.

76. (canceled)

77. The modified immune cell of claim 74, wherein the base editor system comprises a nucleic acid sequence set forth in any one of SEQ ID NOs: 253-258.

78. The modified immune cell of claim 64, wherein the modified endogenous gene locus of CD45 comprises a nucleotide sequence set forth in any one of SEQ ID NOs: 3, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, and 87.

79. The modified immune cell of claim 64, wherein the modified endogenous gene locus of CD45 encodes a polypeptide comprising the amino acid sequence set forth in any one of SEQ ID NOs: 4, 8, 10, 12, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, and 88.

80. The modified immune cell of claim 64, wherein the CAR comprises an antigen binding domain selected from the group consisting of an antibody, an scFv, and a Fab.

81. The modified immune cell of claim 64, wherein the CAR further comprises a hinge domain selected from the group consisting of an Fc fragment of an antibody, a hinge region of an antibody, a CH2 region of an antibody, a CH3 region of an antibody, an artificial hinge domain, a hinge comprising an amino acid sequence of CD8, or any combination thereof.

82. (canceled)

83. The modified immune cell of claim 64, wherein the CAR comprises a transmembrane domain selected from the group consisting of an artificial hydrophobic sequence and transmembrane domain of a type I transmembrane protein, an alpha, beta, or zeta chain of a T cell receptor, CD28, CD3 epsilon, CD45, CD4, CD5, CD8, CD9, CD16, CD22, CD33, CD37, CD64, CD80, CD86, CD134, CD137, and CD154.

84. The modified immune cell of claim 64, wherein the CAR comprises at least one co-stimulatory domain selected from the group consisting of co-stimulatory domains of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3.

85. The modified immune cell of claim 64, wherein the CAR comprises an intracellular domain comprising an intracellular domain selected from the group consisting of cytoplasmic signaling domains of a human CD3 zeta chain, FcγRIII, FcsRI, a cytoplasmic tail of an Fc receptor, an immunoreceptor tyrosine-based activation motif (ITAM) bearing cytoplasmic receptors, TCR zeta, FcR gamma, FcR beta, CD3 gamma, CD3 delta, CD3 epsilon, CD5, CD22, CD79a, CD79b, and CD66d.

86. The modified immune cell of claim 64, wherein the modified cell is resistant to CAR T cell fratricide.

87. The modified immune cell of claim 64, wherein the modified cell is an autologous cell.

88. The modified immune cell of claim 64, wherein the modified cell is a cell isolated from a human subject.

89. The modified immune cell of claim 64, wherein the modified cell is a modified T cell.

90. A method for generating a modified immune cell or precursor cell thereof, comprising:
introducing into an immune or precursor cell a CRISPR system comprising one or more polypeptides and/or nucleic acids capable of modifying expression of endogenous CD45 gene; and
introducing into the immune or precursor cell a nucleic acid encoding a chimeric antigen receptor (CAR), wherein the CAR has affinity for an antigen on a target cell, wherein the CRISPR system comprises a CRISPR nuclease and a guide RNA.

91. The method of claim 90, wherein the one or more polypeptides and/or nucleic acids capable of downregulating gene expression of endogenous CD45 introduces a CRISPR-mediated modification in an endogenous gene locus encoding CD45.

92. The method of claim 91, wherein the modification is selected from the group consisting of a substitution, an insertion, a deletion, and an insertion/deletion.

93. (canceled)

94. The method of claim 90, wherein the CRISPR nuclease is Cas9.

95. The method of claim 90, wherein the guide RNA comprises a guide sequence that is sufficiently complementary with a target sequence in the endogenous gene locus encoding CD45.

96. The method of claim 90, wherein the guide RNA comprises a nucleic acid sequence set forth in any one of SEQ ID NO: 212-223.

97. The method of claim 90, wherein the CRISPR system comprises a base editor system.

98. The method of claim 97, wherein the base editor system comprises a single guide RNA.

99. The method of claim 98, wherein the single guide RNA comprises a nucleic acid sequence set forth in any one of SEQ ID NOs: 224-252.

100. The method of claim 97, wherein the base editor system comprises a nucleic acid sequence set forth in any one of SEQ ID NOs: 253-258.

101. A method of treating cancer in a subject in need thereof, comprising:
i. administering to the subject a modified immune cell of claim 64; and
ii. administering to the subject a modified precursor cell comprising a CRISPR-mediated modification in an endogenous gene locus encoding CD45.

102. The method of claim 101, wherein the modified precursor cell is selected from the group consisting of a bone marrow stem cell, a hematopoietic progenitor cell, and a cord blood stem cell.

103. The method of claim 101, wherein the subject is conditioned by pre-treatment with radiation, chemotherapy, or any combination thereof.

104-105. (canceled)

106. The method of claim 103, wherein the chemotherapy is selected from the group consisting of anti-thymocyte globulin, carmustine, bulsulfan, carboplatin, cyclosporin A, clofarabine, cyclophosphamide, etoposide, fludarabine, melphalan, methotrexate, tacrolimus, thiotepa, topotecan, or any combination thereof.

107. The method of claim 101, wherein the cancer is a hematologic cancer.

108. The method of claim 107, wherein the hematologic cancer is selected from the group consisting of a B-cell lymphoma, a B-cell leukemia, a multiple myeloma, and an acute myeloid leukemia.

109. The method of claim 101, wherein the modified precursor cell is administered first.

110. The method of claim 101, wherein the modified precursor cell and modified immune cell are administered concurrently.

111. A method of conditioning a subject prior to bone marrow transplant, comprising administering to the subject an effective amount of a modified T cell comprising a modified immune cell of claim 64.

112. The method of claim 111, wherein conditioning further comprises administering an effective amount of a chemotherapy, radiation, or a combination thereof.

113. The method of claim 112, wherein the chemotherapy, radiation, or combination thereof is administered prior to administration of the modified T cell or modified immune cell.

114. The method of claim 112, wherein the chemotherapy is selected from the group consisting of anti-thymocyte globulin, carmustine, bulsulfan, carboplatin, cyclosporin A, clofarabine, cyclophosphamide, etoposide, fludarabine, melphalan, methotrexate, tacrolimus, thiotepa, topotecan, or any combination thereof.

115. A method of treating an HIV infection in a subject, comprising:
a. administering to the subject a modified immune cell of claim 64; and
b. administering to the subject a modified precursor cell comprising a CRISPR-mediated modification in a first endogenous gene locus and one or more second endogenous gene loci, thereby treating the HIV infection.

116. The method of claim 115, wherein the first endogenous gene locus is CD45.

117. The method of claim 115, wherein the one or more second endogenous gene loci are selected from the group consisting of CD4, CCR5, CXCR4, and any combination thereof.

118. The method of claim 115, wherein the CRISPR-mediated modification is selected from the group consisting of a substitution, an insertion, a deletion, and an insertion/deletion.

119. The method of claim 115, wherein the CRISPR-mediated modification of the one or more second endogenous gene loci reduces or eliminates the binding of the protein encoded by the loci with HIV gp120.

120. The method of claim 115, wherein the modified precursor cell is selected from the group consisting of a bone marrow stem cell, a hematopoietic progenitor cell, and a cord blood stem cell.

121. The method of claim 115, wherein the subject is conditioned by pre-treatment with radiation, chemotherapy, or any combination thereof.

122-123. (canceled)

124. The method of claim 121, wherein the chemotherapy is selected from the group consisting of anti-thymocyte globulin, carmustine, bulsulfan, carboplatin, cyclosporin A, clofarabine, cyclophosphamide, etoposide, fludarabine, melphalan, methotrexate, tacrolimus, thiotepa, topotecan, or any combination thereof.

125. A method of treating a hematologic malignancy in a subject in need thereof, comprising:
i. administering to the subject a CD33-targeted therapy comprising a CD33-specific CAR-T cell; and
ii. administering to the subject a population of modified precursor cells comprising a modification in an endogenous gene locus such that the precursor cells are resistant to the CD33-targeted therapy.

126. The method of claim 125, wherein the endogenous gene locus is CD33.

127. The method of claim 125, wherein the modification is a CRISPR-mediated modification.

128. The method of claim 125, wherein the CRISPR-mediated modification is selected from the group consisting of a substitution, an insertion, a deletion, and an insertion/deletion.

129. The method of claim 125, wherein the modified precursor cell is selected from the group consisting of a bone marrow stem cell, a hematopoietic progenitor cell, and a cord blood stem cell.

130. The method of claim 125, wherein the subject is conditioned by pre-treatment with radiation, a chemotherapy, or any combination thereof.

131-132. (canceled)

133. The method of claim 130, wherein the chemotherapy is selected from the group consisting of anti-thymocyte globulin, carmustine, bulsulfan, carboplatin, cyclosporin A, clofarabine, cyclophosphamide, etoposide, fludarabine, melphalan, methotrexate, tacrolimus, thiotepa, topotecan, or any combination thereof.

134. The method of claim 125, wherein the hematologic malignancy is a myeloid malignancy selected from the group consisting of acute myelogenous leukemia (AML), chronic myelogenous leukemia (CML), myelodysplastic neoplasm, and myeloproliferative neoplasm.
